SEQUENCE LISTING (196 sequences)

SEQ ID NO: 1 - protein, 595 aa, synthetic construct [sequence omitted]

SEQ ID NO: 2 - protein, 313 aa, synthetic construct
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPAT

SEQ ID NO: 3 - protein, 314 aa, synthetic construct
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATA

SEQ ID NO: 4 - protein, 19 aa, synthetic construct
GGGGSGGGGSGGGGSVDGF

SEQ ID NO: 5 - protein, 9 aa, synthetic construct
ASGGGGSAS

SEQ ID NO: 6 - protein, 176 aa, synthetic construct
SRPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLRGS

SEQ ID NO: 7 - DNA, 31 bp, synthetic construct
  source 1..31: mol_type = other DNA
agagggtatataatggaagctcgacttccag

SEQ ID NO: 8 - DNA, 52 bp, synthetic construct
  source 1..52: mol_type = other DNA
gggaatttccggggactttccgggaatttccggggactttccgggaatttcc

SEQ ID NO: 9 - DNA, 85 bp, synthetic construct
  source 1..85: mol_type = other DNA
caccagacagtgacgtcagctgccagatcccatggccgtcatactgtgacgtctttcagacaccccattgacgtcaatgggagaa

SEQ ID NO: 10 - DNA, 90 bp, synthetic construct
  source 1..90: mol_type = other DNA
ggaggaaaaactgtttcatacagaaggcgtggaggaaaaactgtttcatacagaaggcgtggaggaaaaactgtttcatacagaaggcgt

SEQ ID NO: 11 - DNA, 115 bp, synthetic construct
  source 1..115: mol_type = other DNA
aggatgtccatattaggacatctaggatgtccatattaggacatctaggatgtccatattaggacatctaggatgtccatattaggacatctaggatgtccatattaggacatct

SEQ ID NO: 12 - DNA, 115 bp, synthetic construct
  source 1..115: mol_type = other DNA
agtatgtccatattaggacatctaccatgtccatattaggacatctactatgtccatattaggacatcttgtatgtccatattaggacatctaaaatgtccatattaggacatct

SEQ ID NO: 13 - DNA, 48 bp, synthetic construct
  source 1..48: mol_type = other DNA
tgagtcagtgactcagtgagtcagtgactcagtgagtcagtgactcag

SEQ ID NO: 14 - DNA, 120 bp, synthetic construct
  source 1..120: mol_type = other DNA
agatcaaagggtttaagatcaaagggcttaagatcaaagggtataagatcaaagggcctaagatcaaagggactaagatcaaagggtttaagatcaaagggcttaagatcaaagggccta

SEQ ID NO: 15 - DNA, 32 bp, synthetic construct
  source 1..32: mol_type = other DNA
gtctagacgtctagacgtctagacgtctagac

SEQ ID NO: 16 - DNA, 24 bp, synthetic construct
  source 1..24: mol_type = other DNA
cagacacagacacagacacagaca

SEQ ID NO: 17 - DNA, 65 bp, synthetic construct
  source 1..65: mol_type = other DNA
ggatccggtactcgagatctgcgatctaagtaagcttggcattccggtactgttggtaaagccac

SEQ ID NO: 18 - DNA, 588 bp, synthetic construct
  source 1..588: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 19 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgccgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcgaccaccgagaatcggacgggggtagtctcaagctggccggcctgctctggtgcctgtcctcgcgccgccgtgtatcgccccgccccgggcggcaaggctggcccggtcggcaccagttgcgtgagcggaaagatggccgcttcccggtcctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 20 - DNA, 544 bp, synthetic construct
  source 1..544: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 21 - DNA, 399 bp, synthetic construct
  source 1..399: mol_type = other DNA
tttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagccca

SEQ ID NO: 22 - DNA, 511 bp, synthetic construct
  source 1..511: mol_type = other DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 23 - DNA, 408 bp, synthetic construct
  source 1..408: mol_type = other DNA
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 24 - DNA, 344 bp, synthetic construct
  source 1..344: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagct

SEQ ID NO: 25 - DNA, 1229 bp, synthetic construct
  source 1..1229: mol_type = other DNA
gcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaagcttctgcaggtcgactctagaaaattgtccgctaaattctggccgtttttggcttttttgttagac

SEQ ID NO: 26 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 27 - DNA, 1718 bp, synthetic construct
  source 1..1718: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 28 - DNA, 212 bp, synthetic construct
  source 1..212: mol_type = other DNA
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 29 - DNA, 2379 bp, synthetic construct
  source 1..2379: mol_type = other DNA
ccactagttccatgtccttatatggactcatctttgcctattgcgacacacactcaatgaacacctactacgcgctgcaaagagccccgcaggcctgaggtgcccccacctcaccactcttcctatttttgtgtaaaaatccagcttcttgtcaccacctccaaggagggggaggaggaggaaggcaggttcctctaggctgagccgaatgcccctctgtggtcccacgccactgatcgctgcatgcccaccacctgggtacacacagtctgtgattcccggagcagaacggaccctgcccacccggtcttgtgtgctactcagtggacagacccaaggcaagaaagggtgacaaggacagggtcttcccaggctggctttgagttcctagcaccgccccgcccccaatcctctgtggcacatggagtcttggtccccagagtcccccagcggcctccagatggtctgggagggcagttcagctgtggctgcgcatagcagacatacaacggacggtgggcccagacccaggctgtgtagacccagcccccccgccccgcagtgcctaggtcacccactaacgccccaggcctggtcttggctgggcgtgactgttaccctcaaaagcaggcagctccagggtaaaaggtgccctgccctgtagagcccaccttccttcccagggctgcggctgggtaggtttgtagccttcatcacgggccacctccagccactggaccgctggcccctgccctgtcctggggagtgtggtcctgcgacttctaagtggccgcaagccacctgactcccccaacaccacactctacctctcaagcccaggtctctccctagtgacccacccagcacatttagctagctgagccccacagccagaggtcctcaggccctgctttcagggcagttgctctgaagtcggcaagggggagtgactgcctggccactccatgccctccaagagctccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatccgccgcccgtccacacccgccgccaggtaagcccggccagccgaccggggcaggcggctcacggcccggccgcaggcggccgcggccccttcgcccgtgcagagccgccgtctgggccgcagcggggggcgcatggggggggaaccggaccgccgtggggggcgcgggagaagcccctgggcctccggagatgggggacaccccacgccagttcggaggcgcgaggccgcgctcgggaggcgcgctccgggggtgccgctctcggggcgggggcaaccggcggggtctttgtctgagccgggctcttgccaatggggatcgcagggtgggcgcggcggagcccccgccaggcccggtgggggctggggcgccattgcgcgtgcgcgctggtcctttgggcgctaactgcgtgcgcgctgggaattggcgctaattgcgcgtgcgcgctgggactcaaggcgctaactgcgcgtgcgttctggggcccggggtgccgcggcctgggctggggcgaaggcgggctcggccggaaggggtggggtcgccgcggctcccgggcgcttgcgcgcacttcctgcccgagccgctggccgcccgagggtgtggccgctgcgtgcgcgcgcgccgacccggcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcgggggcgacctcggctcacagcgcgcccggctat

SEQ ID NO: 30 - DNA, 529 bp, synthetic construct
  source 1..529: mol_type = other DNA
gttgatttccttcatccctggcacacgtccaggcagtgtcgaatccatctctgctacaggggaaaacaaataacatttgagtccagtggagaccgggagcagaagtaaagggaagtgataacccccagagcccggaagcctctggaggctgagacctcgccccccttgcgtgatagggcctacggagccacatgaccaaggcactgtcgcctccgcacgtgtgagagtgcagggccccaagatggctgccaggcctcgaggcctgactcttctatgtcacttccgtaccggcgagaaaggcgggccctccagccaatgaggctgcggggcgggccttcaccttgataggcactcgagttatccaatggtgcctgcgggccggagcgactaggaactaacgtcatgccgagttgctgagcgccggcaggcggggccggggcggccaaaccaatgcgatggccggggcggagtcgggcgctctataagttgtcgataggcgggcactccgccctagtttctaaggaccatg

SEQ ID NO: 31 - DNA, 794 bp, synthetic construct
  source 1..794: mol_type = other DNA
agttccccaactttcccgcctctcagcctttgaaagaaagaaaggggagggggcaggccgcgtgcagtcgcgagcggtgctgggctccggctccaattccccatctcagtcgctcccaaagtccttctgtttcatccaagcgtgtaagggtccccgtccttgactccctagtgtcctgctgcccacagtccagtcctgggaaccagcaccgatcacctcccatcgggccaatctcagtcccttcccccctacgtcggggcccacacgctcggtgcgtgcccagttgaaccaggcggctgcggaaaaaaaaaagcggggagaaagtagggcccggctactagcggttttacgggcgcacgtagctcaggcctcaagaccttgggctgggactggctgagcctggcgggaggcggggtccgagtcaccgcctgccgccgcgcccccggtttctataaattgagcccgcagcctcccgcttcgctctctgctcctcctgttcgacagtcagccgcatcttcttttgcgtcgccaggtgaagacgggcggagagaaacccgggaggctagggacggcctgaaggcggcaggggcgggcgcaggccggatgtgttcgcgccgctgcggggtgggcccgggcggcctccgcattgcaggggcgggcggaggacgtgatgcggcgcgggctgggcatggaggcctggtgggggaggggaggggaggcgtgggtgtcggccggggccactaggcgctcactgttctctccctccgcgcagccgagccacatcgctgagacac

SEQ ID NO: 32 - protein, 191 aa, synthetic construct
DGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWKKMG

SEQ ID NO: 33 - DNA, 701 bp, synthetic construct
  source 1..701: mol_type = other DNA
tagtttcatcaccaccgccacccccccgcccccccgccatctgaaagggttctaggggatttgcaacctctctcgtgtgtttcttctttccgagaagcgccgccacacgagaaagctggccgcgaaagtcgtgctggaatcacttccaacgaaaccccaggcatagatgggaaagggtgaagaacacgttgccatggctaccgtttccccggtcacggaataaacgctctctaggatccggaagtagttccgccgcgacctctctaaaaggatggatgtgttctctgcttacattcattggacgttttcccttagaggccaaggccgcccaggcaaaggggcggtcccacgcgtgaggggcccgcggagccatttgattggagaaaagctgcaaaccctgaccaatcggaaggagccacgcttcgggcatcggtcaccgcacctggacagctccgattggtggacttccgccccccctcacgaatcctcattgggtgccgtgggtgcgtggtgcggcgcgattggtgggttcatgtttcccgtcccccgcccgcgagaagtgggggtgaaaagcggcccgacctgcttggggtgtagtgggcggaccgcgcggctggaggtgtgaggatccgaacccaggggtggggggtggaggcggctcctgcgatcgaaggggacttgagactcaccggccgcacgtc

SEQ ID NO: 34 - DNA, 465 bp, synthetic construct
  source 1..465: mol_type = other DNA
gggccgcccactcccccttcctctcagggtccctgtcccctccagtgaatcccagaagactctggagagttctgagcagggggcggcactctggcctctgattggtccaaggaaggctggggggcaggacgggaggcgaaaaccctggaatattcccgacctggcagcctcatcgagctcggtgattggctcagaagggaaaaggcgggtctccgtgacgacttataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaaccc

SEQ ID NO: 35 - DNA, 538 bp, synthetic construct
  source 1..538: mol_type = other DNA
gccccacccccgtccgcgttacaaccgggaggcccgctgggtcctgcaccgtcaccctcctccctgtgaccgcccacctgatacccaaacaactttctcgcccctccagtccccagctcgccgagcgcttgcggggagccacccagcctcagtttccccagccccgggcggggcgaggggcgatgacgtcatgccggcgcgcggcattgtggggcggggcgaggcggggcgccggggggagcaacactgagacgccattttcggcggcgggagcggcgcaggcggccgagcgggactggctgggtcggctgggctgctggtgcgaggagccgcggggctgtgctcggcggccaaggggacagcgcgtgggtggccgaggatgctgcggggcggtagctccggcgcccctcgctggtgactgctgcgccgtgcctcacacagccgaggcgggctcggcgcacagtcgctgctccgcgctcgcgcccggcggcgctccaggtgctgacagcgcgagagagcgcggcctcaggagcaacac

SEQ ID NO: 36 - DNA, 1091 bp, synthetic construct
  source 1..1091: mol_type = other DNA
ttccagagctttcgaggaaggtttcttcaactcaaattcatccgcctgataattttcttatattttcctaaagaaggaagagaagcgcatagaggagaagggaaataattttttaggagcctttcttacggctatgaggaatttggggctcagttgaaaagcctaaactgcctctcgggaggttgggcgcggcgaactactttcagcggcgcacggagacggcgtctacgtgaggggtgataagtgacgcaacactcgttgcataaatttgcgctccgccagcccggagcatttaggggcggttggctttgttgggtgagcttgtttgtgtccctgtgggtggacgtggttggtgattggcaggatcctggtatccgctaacaggtactggcccacagccgtaaagacctgcgggggcgtgagaggggggaatgggtgaggtcaagctggaggcttcttggggttgggtgggccgctgaggggaggggagggcgaggtgacgcgacacccggcctttctgggagagtgggccttgttgacctaaggggggcgagggcagttggcacgcgcacgcgccgacagaaactaacagacattaaccaacagcgattccgtcgcgtttacttgggaggaaggcggaaaagaggtagtttgtgtggcttctggaaaccctaaatttggaatcccagtatgagaatggtgtcccttcttgtgtttcaatgggatttttacttcgcgagtcttgtgggtttggttttgttttcagtttgcctaacaccgtgcttaggtttgaggcagattggagttcggtcgggggagtttgaatatccggaacagttagtggggaaagctgtggacgcttggtaagagagcgctctggattttccgctgttgacgttgaaaccttgaatgacgaatttcgtattaagtgacttagccttgtaaaattgaggggaggcttgcggaatattaacgtatttaaggcattttgaaggaatagttgctaattttgaagaatattaggtgtaaaagcaagaaatacaatgatcctgaggtgacacgcttatgttttacttttaaactaggtcacc

SEQ ID NO: 37 - DNA, 243 bp, synthetic construct
  source 1..243: mol_type = other DNA
cgggtttcgtaacaatcgcatgaggattcgcaacgccttcggcgtagccgatgtcgcgctcccgtctcagtaaaggtcggcgtagccgatgtcgcgcaatcggactgccttcgtacggcgtagccgatgtcgcgcgtatcagtcgcctcggaacggcgtagccgatgtcgcgcattcgtaagaggctcactctcccttacacggagtggataactagttctagagggtatataatgggggcca

SEQ ID NO: 38 - DNA, 672 bp, synthetic construct
  source 1..672: mol_type = other DNA
tctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttccatggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctgtttcgcctcggcgtccggtccgtgttgcttggtcgtcacctgtgcagaattgcgaaccatggattcca

SEQ ID NO: 39 - protein, 415 aa, synthetic construct
DEADRRLLRRCRLRLVEELQVDQLWDVLLSRELFRPHMIEDIQRAGSGSRRDQARQLIIDLETRGSQALPLFISCLEDTGQDMLASFLRTNRQAAKLSKPTLENLTPVVLRPEIRKPEVLRPETPRPVDIGSGGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 40 - protein, 312 aa, synthetic construct
AGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPAT

SEQ ID NO: 41 - protein, 217 aa, synthetic construct
DPDDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSLCEGTLLLWCDIIGQTTYRDLKL

SEQ ID NO: 42 - protein, 312 aa, synthetic construct; identical to SEQ ID NO: 40

SEQ ID NO: 43 - protein, 312 aa, synthetic construct
AGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWMEILMIGVVWRSMEHPVKLLFAPNLLLDRNQGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPAT

SEQ ID NO: 44 - protein, 312 aa, synthetic construct
AGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWMEILMIGLVWRSMEHPVKLLFAPNLVLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMEFLYSMKCKNVVPLYDLLLEAADAHRLHAPTSRGGASVEETDQSHLATAGSTSSHSLQKYYITGEAEGFPAT

SEQ ID NO: 45 - protein, 19 aa, synthetic construct
GGGGSGGGGSGGGGSVDGF

SEQ ID NO: 46 - protein, 9 aa, synthetic construct
ASGGGGSAS

SEQ ID NO: 47 - protein, 246 aa, synthetic construct
QPILLLLAFLLLPRADAGEIIGGHEAKPHSRPYMAYLMIWDQKSLKRCGGFLIQDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRH

SEQ ID NO: 48 - protein, 279 aa, synthetic construct
DVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 49 - protein, 312 aa, synthetic construct
AGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLVLDRDEGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPAT

SEQ ID NO: 50 - protein, 312 aa, synthetic construct
AGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLLLDRDEGKCVEGLVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHASNKGMEFLYSMKCKNVVPLYDLLLEAADAHRLHAPTSRGGASVEETDQSHLATAGSTSSHSLQKYYITGEAEGFPAT

SEQ ID NO: 51 - protein, 312 aa, synthetic construct
AGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGLVWRSMEHPVKLLFAPNLVLDRNQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPAT

SEQ ID NO: 52 - protein, 312 aa, synthetic construct
AGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLVLDRDEGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPAT

SEQ ID NO: 53 - protein, 312 aa, synthetic construct
AGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNEGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPAT

SEQ ID NO: 54 - protein, 194 aa, synthetic construct
DCEVNNGSSLRDECITNLLVFGFLQSCSDNSFRRELDALGHELPVLAPQWEGYDELQTDGNRSSHSRLGRIEADSESQEDIIRNIARHLAQVGDSMDRSIPPGLVNGLALQLRNTSRSEEDRNRDLATALEQLLQAYPRDMEKEKTMLVLALLLAKKVASHTPSLLRDVFHTTVNFINQNLRTYVRSLARNGMD

SEQ ID NO: 55 - protein, 531 aa, synthetic construct
EASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 56 - protein, 503 aa, synthetic construct
SRGSEFMTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 57 - protein, 176 aa, synthetic construct; identical to SEQ ID NO: 6

SEQ ID NO: 58 - protein, 541 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 59 - DNA, 212 bp, synthetic construct
  source 1..212: mol_type = other DNA
cgggtttcgtaacaatcgcatgaggattcgcaacgccttcggcgtagccgatgtcgcgctcccgtctcagtaaaggtcggcgtagccgatgtcgcgcaatcggactgccttcgtacggcgtagccgatgtcgcgcgtatcagtcgcctcggaacggcgtagccgatgtcgcgcattcgtaagaggctcactctcccttacacggagtggata

SEQ ID NO: 60 - DNA, 573 bp, synthetic construct
  source 1..573: mol_type = other DNA
gacgggtccggggagcagcccagaggcggggggcccaccagctctgagcagatcatgaagacaggggcccttttgcttcagggtttcatccaggatcgagcagggcgaatgggtggagaggcacccgagctggccctggacccggtgcctcaggatgcgtccaccaagaagctgagcgagtgtctcaagcgcatcggggacgaactggacagtaacatggagctgcagaggatgattgccgccgtggacacagactccccccgagaggtctttttccgagtggcagctgacatgttttctgacggcaacttcaactggggccgggttgtcgcccttttctactttgccagcaaactggtgctcaaggccctgtgcaccaaggtgccggaactgatcagaaccatcatgggctggacattggacttcctccgggagcggctgttgggctggatccaagaccagggtggttgggacggcctcctctcctactttgggacgcccacgtggcagaccgtgaccatctttgtggcgggagtgctcaccgcctcactcaccatctggaagaagatgggc

SEQ ID NO: 61 - DNA, 531 bp, synthetic construct
  source 1..531: mol_type = other DNA
atgtctagacctggcgagaggcccttccagtgccggatctgcatgcggaacttcagcaacatgagcaacctgaccagacacacccggacacacacaggcgagaagccttttcagtgcagaatctgtatgcgcaatttctccgacagaagcgtgctgcggagacacctgagaacccacaccggcagccagaaaccattccagtgtcgcatctgtatgagaaactttagcgacccctccaatctggcccggcacaccagaacacataccggggaaaaaccctttcagtgtaggatatgcatgaggaatttttccgaccggtccagcctgaggcggcacctgaggacacatactggctcccaaaagccgttccaatgtcggatatgtatgcgcaactttagccagagcggcaccctgcacagacacacaagaacccatactggcgagaaacctttccaatgtagaatctgcatgcgaaatttttcccagcggcctaatctgaccaggcatctgaggacccacctgagaggatct

SEQ ID NO: 62 - protein, 177 aa, synthetic construct
MSRPGERPFQCRICMRNFSNMSNLTRHTRTHTGEKPFQCRICMRNFSDRSVLRRHLRTHTGSQKPFQCRICMRNFSDPSNLARHTRTHTGEKPFQCRICMRNFSDRSSLRRHLRTHTGSQKPFQCRICMRNFSQSGTLHRHTRTHTGEKPFQCRICMRNFSQRPNLTRHLRTHLRGS

SEQ ID NO: 63 - DNA, 573 bp, synthetic construct
  source 1..573: mol_type = other DNA
gatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcc

SEQ ID NO: 64 - protein, 191 aa, synthetic construct
DEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 65 - DNA, 936 bp, synthetic construct
  source 1..936: mol_type = other DNA
gctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggatggagatcctgatgattggtgtggtctggcgctccatggagcacccagtgaagctactgtttgctcctaacttgctcttggacagggaccagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccaca

SEQ ID NO: 66 - protein, 312 aa, synthetic construct
AGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGVVWRSMEHPVKLLFAPNLLLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPAT

SEQ ID NO: 67 - DNA, 1245 bp, synthetic construct
  source 1..1245: mol_type = other DNA
gacgaagcggatcggcggctcctgcggcggtgccggctgcggctggtggaagagctgcaggtggaccagctctgggacgtcctgctgagccgcgagctgttcaggccccatatgatcgaggacatccagcgggcaggctctggatctcggcgggatcaggccaggcagctgatcatagatctggagactcgagggagtcaggctcttcctttgttcatctcctgcttagaggacacaggccaggacatgctggcttcgtttctgcgaactaacaggcaagcagcaaagttgtcgaagccaaccctagaaaaccttaccccagtggtgctcagaccagagattcgcaaaccagaggttctcagaccggaaacacccagaccagtggacattggttctggaggattcggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 68 - DNA, 936 bp, synthetic construct
  source 1..936: mol_type = other DNA
gctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagatcgttcacatgatcaactgggcgaagagggtgccaggctttgtggatttgaccctccatgatcaggtccaccttctagaatgtgcctggctagagatcctgatgattggtgtggtctggcgctccatggagcacccagtgaagctactgtttgctcctaacttgctcttggacagggaccagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacgccagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccaca

SEQ ID NO: 69 - DNA, 651 bp, synthetic construct
  source 1..651: mol_type = other DNA
gaccctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctc

SEQ ID NO: 70 - DNA, 936 bp, synthetic construct
  source 1..936: mol_type = other DNA
gccggcgatatgagagctgctaacctgtggccttctccactgatgatcaagcggtccaagaagaacagtctggccctgagcctgaccgccgaccagatggtttcagcactgctggatgccgagcctcctatcctgtacagcgagtacgaccccaccagaccttttagcgaggccagcatgatgggcctgctgaccaatctggccgacagagaactggtgcacatgatcaactgggccaagcgcgtgcccggctttgtggatctgacactgcacgaccaagtgcatctgctcgagtgcgcctggctggaaatcctgatgatcggactcgtgtggcggagcatggaacaccctgtgaagctgctgttcgcccctaacctgctgctggacagaaaccagggcaaatgcgtggaaggcatggtggaaatcttcgatatgctgctggccacctccagccggttccggatgatgaatctgcagggcgaagagttcgtgtgcctgaagtccattatcctgctgaacagcggcgtgtacacctttctgagcagcaccctgaagtctctggaagagaaggaccacatccacagagtgctggacaagatcaccgacacactgatccacctgatggccaaggccggactgactctgcagcagcagcatcaaagactggcccagctgctgctcatcctgagccacatcagacacatgagcaacaaaggcatggaacatctgtacagcatgaagtgcaagaacgtggtgcccctgtacgatctgctgctcgaagccgctgatgcccacagactgcatgcccctacatctagaggcggagcctccgtggaagagacagatcagtctcatctggccaccgccggcagcacaagctctcattctctgcagaagtactacatcaccggcgaggccgagggatttcctgccaca

SEQ ID NO: 71 - DNA, 936 bp, synthetic construct
  source 1..936: mol_type = other DNA
gctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttggtgttggacagggacgagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccaca

SEQ ID NO: 72 - protein, 312 aa, synthetic construct; identical to SEQ ID NO: 49

SEQ ID NO: 73 - DNA, 57 bp, synthetic construct
  source 1..57: mol_type = other DNA
gggggtggaggttcagggggtggaggttcaggtggtggcggtagtgtcgatggcttc

SEQ ID NO: 74 - DNA, 27 bp, synthetic construct
  source 1..27: mol_type = other DNA
gctagtggcggcggcggcagtgctagt

SEQ ID NO: 75 - DNA, 738 bp, synthetic construct
  source 1..738: mol_type = other DNA
caaccaatcctgcttctgctggccttcctcctgctgcccagggcagatgcaggggagatcatcgggggacatgaggccaagccccactcccgcccctacatggcttatcttatgatctgggatcagaagtctctgaagaggtgcggtggcttcctgatacaagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgccac

SEQ ID NO: 76 - DNA, 837 bp, synthetic construct
  source 1..837: mol_type = other DNA
gatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 77 - protein, 46 aa, synthetic construct
LLPSWAITLISVNGIFVICCLTYCFAPRCRERRRNERLRRESVRPV

SEQ ID NO: 78 - DNA, 330 bp, synthetic construct
  source 1..330: mol_type = other DNA
gtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaa

SEQ ID NO: 79 - DNA, 11892 bp, synthetic construct
  source 1..11892: mol_type = other DNA
aagcttgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgagtccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttacttacacaggccgcacagattctctccgcagccgttcgtttcttctccgctctctgcaccgaggggcgaagcagtaggtcaggcaacagatcacgaagatgccgttcacggagatcagtgtgatggcccagcttggcagcagttgaagagatccaccgccactgccaccgccgccactaccgccaccaaagaagctggagattgtagacacggcgagtccctgtgtaattccagatcctccgcctccgctaccacctccgccgctagaggcgttcaggtagctcatcactctgtcgatggtcacggctctgatccggaaggcgtgcagcaggatgcacagcttgatcttggtcttgtagaagtcgggttcttccaggctagacttctggggcactgtctcgctgttgaagttcagggcctgcatcagctcgtcgatcacggccagcatattctggtccaggaagatctgccgcttggggtccatcagcagcttggcgttcatggtcttgaattccacctggtacatcttcaggtcctcgtagatgctgctcaggcacagggccatcatgaaggaggtctttctgctggccaggcaagagccgttggtgatgaagctggtttcccggctgttcaggcagctctcgttcttggtcagttccagaggcaggcaggcttccacggtgctggtcttatccttggtgatgtcctcgtggtcgatttcctcgctggtgcaggggtagaattccagggtctgtctggccttctgcagcatgttggacacggctctcagcaggttctggctgtggtgcagacaagggaacatgccaggatcaggagtggccacaggcaggtttctagatccgccgccagatccaccacctgatccgccaccgcttcctccgccagaacatggcacgctggcccattcgctccaagagctgctgtagtaccggtcctgggctctgacgctgatgctggcgttctttctgcagatcacggtggcgctggtcttgtcggtgaacacccggtcctttttctcgcgcttggacttgccctgcacttgcacgcaaaaggtcaggctgaagtagctgtggggtgtagaccaggtgtcggggtactcccaggacacttccacctgtctgctgttcttcagaggcttcagctgcaggttctttggaggatcgggcttgatgatgtcccggatgaaaaagctggaggtgtagttctcgtacttcagcttgtgcacggcgtccaccatcacttcgataggcagagactcttcggcggctggacaggcgctgtcctcttggcattccacgctgtactcgtattctttgttgtcgccccgcactctttcggcagacagtgtagcggcgccacatgtaacgccctgaggatcactgctgcctctgctggacttcacgctgaaggtcaggtcggtgctgatggtggtcagccaccaacatgtgaaccggccgctgtagttcttggcctcgcatctcaggaaggtcttgttcttgggctctttctggtccttcaggatgtcggtgctccaaatgccatcctctttcttgtggagcagcagcaggctgtggctcagcacttctccgcctttgtgacaggtgtactggccggcgtcgccaaactctttcacttggatggtcagggtcttgccgctgccgagcacctcgctagactgatccagtgtccaggtgatgccgtcctcttcaggggtatcgcaggtcagcaccaccatctcgccaggagcatcgggataccagtccagttccaccacgtacacgtctttcttcagctcccagatggccaccagaggagaggccaggaacaccaggctgaaccagctgatgaccagctgctggtgacacatcatggtggcgacaccggtacgcgttggcccccattatataccctctagaactagttatccactccgtgtaagggagagtgagcctcttacgaatcttcggcgtcgactgcttcattccgaggcgactgataccttcggcgtcgactgcttcatacgaaggcagtccgattcttcggcgtcgactgcttcaacctttactgagacgggacttcggcgtcgactgcttcaaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgtccatctagatggccgataaaataaaagattttatttagtctccagaaaaaggggggaatgaaagaccccacctgtaggtttggcaagctagctgcagtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaggatccgccaccatgtctagacctggcgagaggcccttccagtgccggatctgcatgcggaacttcagcaacatgagcaacctgaccagacacacccggacacacacaggcgagaagccttttcagtgcagaatctgtatgcgcaatttctccgacagaagcgtgctgcggagacacctgagaacccacaccggcagccagaaaccattccagtgtcgcatctgtatgagaaactttagcgacccctccaatctggcccggcacaccagaacacataccggggaaaaaccctttcagtgtaggatatgcatgaggaatttttccgaccggtccagcctgaggcggcacctgaggacacatactggctcccaaaagccgttccaatgtcggatatgtatgcgcaactttagccagagcggcaccctgcacagacacacaagaacccatactggcgagaaacctttccaatgtagaatctgcatgcgaaatttttcccagcggcctaatctgaccaggcatctgaggacccacctgagaggatctacctgcagggatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcccaattgtgcgtacgcggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggatggagatcctgatgattggtgtggtctggcgctccatggagcacccagtgaagctactgtttgctcctaacttgctcttggacagggaccagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacataagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctacgccttcgccctcagacgagtcggatctccctttgggccgcctccccgcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 80 - DNA, 11892 bp, synthetic construct
  source 1..11892: mol_type = other DNA
aagcttgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgagtccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttacttacacaggccgcacagattctctccgcagccgttcgtttcttctccgctctctgcaccgaggggcgaagcagtaggtcaggcaacagatcacgaagatgccgttcacggagatcagtgtgatggcccagcttggcagcagttgaagagatccaccgccactgccaccgccgccactaccgccaccaaagaagctggagattgtagacacggcgagtccctgtgtaattccagatcctccgcctccgctaccacctccgccgctagaggcgttcaggtagctcatcactctgtcgatggtcacggctctgatccggaaggcgtgcagcaggatgcacagcttgatcttggtcttgtagaagtcgggttcttccaggctagacttctggggcactgtctcgctgttgaagttcagggcctgcatcagctcgtcgatcacggccagcatattctggtccaggaagatctgccgcttggggtccatcagcagcttggcgttcatggtcttgaattccacctggtacatcttcaggtcctcgtagatgctgctcaggcacagggccatcatgaaggaggtctttctgctggccaggcaagagccgttggtgatgaagctggtttcccggctgttcaggcagctctcgttcttggtcagttccagaggcaggcaggcttccacggtgctggtcttatccttggtgatgtcctcgtggtcgatttcctcgctggtgcaggggtagaattccagggtctgtctggccttctgcagcatgttggacacggctctcagcaggttctggctgtggtgcagacaagggaacatgccaggatcaggagtggccacaggcaggtttctagatccgccgccagatccaccacctgatccgccaccgcttcctccgccagaacatggcacgctggcccattcgctccaagagctgctgtagtaccggtcctgggctctgacgctgatgctggcgttctttctgcagatcacggtggcgctggtcttgtcggtgaacacccggtcctttttctcgcgcttggacttgccctgcacttgcacgcaaaaggtcaggctgaagtagctgtggggtgtagaccaggtgtcggggtactcccaggacacttccacctgtctgctgttcttcagaggcttcagctgcaggttctttggaggatcgggcttgatgatgtcccggatgaaaaagctggaggtgtagttctcgtacttcagcttgtgcacggcgtccaccatcacttcgataggcagagactcttcggcggctggacaggcgctgtcctcttggcattccacgctgtactcgtattctttgttgtcgccccgcactctttcggcagacagtgtagcggcgccacatgtaacgccctgaggatcactgctgcctctgctggacttcacgctgaaggtcaggtcggtgctgatggtggtcagccaccaacatgtgaaccggccgctgtagttcttggcctcgcatctcaggaaggtcttgttcttgggctctttctggtccttcaggatgtcggtgctccaaatgccatcctctttcttgtggagcagcagcaggctgtggctcagcacttctccgcctttgtgacaggtgtactggccggcgtcgccaaactctttcacttggatggtcagggtcttgccgctgccgagcacctcgctagactgatccagtgtccaggtgatgccgtcctcttcaggggtatcgcaggtcagcaccaccatctcgccaggagcatcgggataccagtccagttccaccacgtacacgtctttcttcagctcccagatggccaccagaggagaggccaggaacaccaggctgaaccagctgatgaccagctgctggtgacacatcatggtggcgacaccggtacgcgttggcccccattatataccctctagaactagttatccactccgtgtaagggagagtgagcctcttacgaatcttcggcgtcgactgcttcattccgaggcgactgataccttcggcgtcgactgcttcatacgaaggcagtccgattcttcggcgtcgactgcttcaacctttactgagacgggacttcggcgtcgactgcttcaaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgtccatctagatggccgataaaataaaagattttatttagtctccagaaaaaggggggaatgaaagaccccacctgtaggtttggcaagctagctgcagtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaggatccgccaccatgtctagacctggcgagaggcccttccagtgccggatctgcatgcggaacttcagcaacatgagcaacctgaccagacacacccggacacacacaggcgagaagccttttcagtgcagaatctgtatgcgcaatttctccgacagaagcgtgctgcggagacacctgagaacccacaccggcagccagaaaccattccagtgtcgcatctgtatgagaaactttagcgacccctccaatctggcccggcacaccagaacacataccggggaaaaaccctttcagtgtaggatatgcatgaggaatttttccgaccggtccagcctgaggcggcacctgaggacacatactggctcccaaaagccgttccaatgtcggatatgtatgcgcaactttagccagagcggcaccctgcacagacacacaagaacccatactggcgagaaacctttccaatgtagaatctgcatgcgaaatttttcccagcggcctaatctgaccaggcatctgaggacccacctgagaggatctacctgcagggatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcccaattgtgcgtacgcggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagatcgttcacatgatcaactgggcgaagagggtgccaggctttgtggatttgaccctccatgatcaggtccaccttctagaatgtgcctggctagagatcctgatgattggtgtggtctggcgctccatggagcacccagtgaagctactgtttgctcctaacttgctcttggacagggaccagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacgccagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacataagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctacgccttcgccctcagacgagtcggatctccctttgggccgcctccccgcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 81 - DNA, 11892 bp, synthetic construct
  source 1..11892: mol_type = other DNA
aagcttgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgagtccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttacttacacaggccgcacagattctctccgcagccgttcgtttcttctccgctctctgcaccgaggggcgaagcagtaggtcaggcaacagatcacgaagatgccgttcacggagatcagtgtgatggcccagcttggcagcagttgaagagatccaccgccactgccaccgccgccactaccgccaccaaagaagctggagattgtagacacggcgagtccctgtgtaattccagatcctccgcctccgctaccacctccgccgctagaggcgttcaggtagctcatcactctgtcgatggtcacggctctgatccggaaggcgtgcagcaggatgcacagcttgatcttggtcttgtagaagtcgggttcttccaggctagacttctggggcactgtctcgctgttgaagttcagggcctgcatcagctcgtcgatcacggccagcatattctggtccaggaagatctgccgcttggggtccatcagcagcttggcgttcatggtcttgaattccacctggtacatcttcaggtcctcgtagatgctgctcaggcacagggccatcatgaaggaggtctttctgctggccaggcaagagccgttggtgatgaagctggtttcccggctgttcaggcagctctcgttcttggtcagttccagaggcaggcaggcttccacggtgctggtcttatccttggtgatgtcctcgtggtcgatttcctcgctggtgcaggggtagaattccagggtctgtctggccttctgcagcatgttggacacggctctcagcaggttctggctgtggtgcagacaagggaacatgccaggatcaggagtggccacaggcaggtttctagatccgccgccagatccaccacctgatccgccaccgcttcctccgccagaacatggcacgctggcccattcgctccaagagctgctgtagtaccggtcctgggctctgacgctgatgctggcgttctttctgcagatcacggtggcgctggtcttgtcggtgaacacccggtcctttttctcgcgcttggacttgccctgcacttgcacgcaaaaggtcaggctgaagtagctgtggggtgtagaccaggtgtcggggtactcccaggacacttccacctgtctgctgttcttcagaggcttcagctgcaggttctttggaggatcgggcttgatgatgtcccggatgaaaaagctggaggtgtagttctcgtacttcagcttgtgcacggcgtccaccatcacttcgataggcagagactcttcggcggctggacaggcgctgtcctcttggcattccacgctgtactcgtattctttgttgtcgccccgcactctttcggcagacagtgtagcggcgccacatgtaacgccctgaggatcactgctgcctctgctggacttcacgctgaaggtcaggtcggtgctgatggtggtcagccaccaacatgtgaaccggccgctgtagttcttggcctcgcatctcaggaaggtcttgttcttgggctctttctggtccttcaggatgtcggtgctccaaatgccatcctctttcttgtggagcagcagcaggctgtggctcagcacttctccgcctttgtgacaggtgtactggccggcgtcgccaaactctttcacttggatggtcagggtcttgccgctgccgagcacctcgctagactgatccagtgtccaggtgatgccgtcctcttcaggggtatcgcaggtcagcaccaccatctcgccaggagcatcgggataccagtccagttccaccacgtacacgtctttcttcagctcccagatggccaccagaggagaggccaggaacaccaggctgaaccagctgatgaccagctgctggtgacacatcatggtggcgacaccggtacgcgttggcccccattatataccctctagaactagttatccactccgtgtaagggagagtgagcctcttacgaatcttcggcgtcgactgcttcattccgaggcgactgataccttcggcgtcgactgcttcatacgaaggcagtccgattcttcggcgtcgactgcttcaacctttactgagacgggacttcggcgtcgactgcttcaaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgtccatctagatggccgataaaataaaagattttatttagtctccagaaaaaggggggaatgaaagaccccacctgtaggtttggcaagctagctgcagtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaggatccgccaccatgtctagacctggcgagaggcccttccagtgccggatctgcatgcggaacttcagcaacatgagcaacctgaccagacacacccggacacacacaggcgagaagccttttcagtgcagaatctgtatgcgcaatttctccgacagaagcgtgctgcggagacacctgagaacccacaccggcagccagaaaccattccagtgtcgcatctgtatgagaaactttagcgacccctccaatctggcccggcacaccagaacacataccggggaaaaaccctttcagtgtaggatatgcatgaggaatttttccgaccggtccagcctgaggcggcacctgaggacacatactggctcccaaaagccgttccaatgtcggatatgtatgcgcaactttagccagagcggcaccctgcacagacacacaagaacccatactggcgagaaacctttccaatgtagaatctgcatgcgaaatttttcccagcggcctaatctgaccaggcatctgaggacccacctgagaggatctacctgcagggatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcccaattgtgcgtacgcggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttggtgttggacagggacgagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacataagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctacgccttcgccctcagacgagtcggatctccctttgggccgcctccccgcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 82 - DNA, 1593 bp, synthetic construct
  source 1..1593: mol_type = other DNA
gaagcctctggaagcggcagagctgacgccctggatgacttcgacctggatatgctgggcagcgacgctctggacgattttgacctcgacatgctgggatctgatgcactcgacgatttcgatttggacatgctcggcagtgatgccttggacgactttgatcttgatatgctcatcaacagccggtccagcggcagccccaagaaaaaaagaaaagtgggctcccagtacctgcctgacaccgacgacagacaccggatcgaggaaaagcggaagcggacctacgagacattcaagagcatcatgaagaagtccccattcagcggccccaccgatcctagacctccacctagaagaatcgccgtgcctagcagatctagcgcctccgtgcctaaacctgctcctcagccttatcctttcaccagcagcctgagcaccatcaactacgacgagttccctaccatggtgttccccagcggccagatctctcaggcttctgctcttgctccagctcctcctcaggttctgcctcaagctcctgcaccagcaccggctccagctatggtttctgctttggctcaggcccctgctcctgtgcctgttcttgctcctggaccacctcaggctgttgctcctcctgctccaaaacctacacaggccggcgaaggcacactgtctgaagctctgctgcagctccagttcgatgacgaagatctgggcgccctgctgggcaattctacagatcctgccgtgtttaccgatctggccagcgtggacaacagcgagtttcagcagctcctgaatcagggcatccctgtggctcctcacaccaccgaacctatgctgatggaataccccgaggccatcaccagactggtcaccggtgctcaaagaccacctgatccagctccagcaccactgggagcacctggactgcctaatggactgctgtctggcgacgaggacttcagctctatcgccgacatggatttctctgccctgctcggctctggcagcggctctagagatagcagagaaggcatgttcctgcctaagcctgaggccggctctgccatctccgatgtgttcgagggaagagaagtgtgccagcctaagcggatccggccttttcaccctcctggaagcccttgggccaacagacctctgcctgcttctctggcccctacaccaacaggacctgtgcacgaacctgtgggcagtctgaccccagctcctgttcctcaacctctggatcccgctcctgctgtgacacctgaagcctctcatctgctggaagatcccgacgaagagacaagccaggccgtgaaggccctgagagaaatggccgacacagtgatccctcagaaagaggaagccgccatctgcggacagatggacctgtctcatcctccaccaagaggccacctggacgagctgacaaccacactggaatccatgaccgaggacctgaacctggacagccctctgacacccgagctgaacgagatcctggacaccttcctgaacgacgagtgtctgctgcacgccatgcacatctctaccggcctgagcatcttcgacaccagcctgttt

SEQ ID NO: 83 - DNA, 1509 bp, synthetic construct
  source 1..1509: mol_type = other DNA
tctagaggatccgaattcatgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagataggtggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtct

SEQ ID NO: 84 - protein, 175 aa, synthetic construct
MPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLRGS

SEQ ID NO: 85 - DNA, 525 bp, synthetic construct
  source 1..525: mol_type = other DNA
atgcccggagagcgcccattccagtgtcggatttgcatgcggaacttttcgagaagacacggcctggacagacatacccgtactcatacaggtgaaaaaccctttcagtgtcggatctgtatgcgaaatttctccgaccacagcagcctgaagagacatctacgtacccacaccggcagccagaagccatttcagtgtcggatctgtatgcggaacttctccgtgagacacaacctgaccagacatctacgtacgcacaccggagagaagccattccaatgccgaatatgcatgcgcaacttcagtgaccacagcaacctgagcagacacctaaaaacccacaccggttcccagaagccatttcagtgtcggatctgtatgcggaacttctcccagcgcagcagcctggtgagacatctacgtacgcacaccggagagaagccattccaatgccgaatatgcatgcgcaacttcagtgagagcggccacctgaagagacacctgcgtacgcacctgaggggatcc

SEQ ID NO: 86-87 - skipped (2 entries)

SEQ ID NO: 88 - protein, 8 aa, synthetic construct
QLCVRGSS

SEQ ID NO: 89 - DNA, 24 bp, synthetic construct
  source 1..24: mol_type = other DNA
caattgtgcgtacgcggatcctct

SEQ ID NO: 90 - protein, 314 aa, synthetic construct
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGVVWRSMEHPVKLLFAPNLVLDRDEGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPATA

SEQ ID NO: 91 - protein, 314 aa, synthetic construct
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPVKLLFAPNLVLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATA

SEQ ID NO: 92 - protein, 314 aa, synthetic construct
SAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLVLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPATA

SEQ ID NO: 93 - protein, 314 aa, synthetic construct
SAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWMEILMIGVVWRSMEHPVKLLFAPNLLLDRDEGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATA

SEQ ID NO: 94 - protein, 314 aa, synthetic construct
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGVVWRSMEHPVKLLFAPNLVLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPATA

SEQ ID NO: 95 - protein, 314 aa, synthetic construct
SAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLVLDRDEGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATA

SEQ ID NO: 96 - protein, 314 aa, synthetic construct
SAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWMEILMIGVVWRSMEHPVKLLFAPNLVLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATA

SEQ ID NO: 97 - protein, 314 aa, synthetic construct
SAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLLLDRDEGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPATA

SEQ ID NO: 98 - protein, 314 aa, synthetic construct
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGVVWRSMEHPVKLLFAPNLLLDRDQGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPATA

SEQ ID NO: 99 - protein, 314 aa, synthetic construct
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGVVWRSMEHPVKLLFAPNLVLDRDQGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPATA

SEQ ID NO: 100 - protein, 314 aa, synthetic construct
SAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWMEILMIGLVWRSMEHPVKLLFAPNLLLDRDQGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATA

SEQ ID NO: 101 - protein, 314 aa, synthetic construct
SAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGLVWRSMEHPVKLLFAPNLLLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATA

SEQ ID NO: 102 - protein, 314 aa, synthetic construct
SAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLLLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPATA

SEQ ID NO: 103 - protein, 315 aa, synthetic construct
MSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGVVWRSMEHPVKLLFAPNLVLDRDEGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPATA

SEQ ID NO: 104 - protein, 315 aa, synthetic construct
MSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPVKLLFAPNLVLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATA

SEQ ID NO: 105 - protein, 315 aa, synthetic construct
MSAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLVLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPATA

SEQ ID NO: 106 - protein, 315 aa, synthetic construct
MSAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWMEILMIGVVWRSMEHPVKLLFAPNLLLDRDEGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATA

SEQ ID NO: 107 - protein, 497 aa, synthetic construct
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 108 - DNA, 1491 bp, synthetic construct
  source 1..1491: mol_type = other DNA
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagataggtggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtct

SEQ ID NO: 109 - protein, 497 aa, synthetic construct
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGMSLSDWKPSEDPWEQHAKWYSGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 110 - DNA, 1491 bp, synthetic construct
  source 1..1491: mol_type = other DNA
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagataggtggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaatgagcctaagcgattggaagcccagtgaagacccttgggaacaacatgctaaatggtatagcgggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtct

SEQ ID NO: 111 - protein, 497 aa, synthetic construct
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGMSLDDWKPSEDPWEQHAKWYSGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 112 - DNA, 1491 bp, synthetic construct
  source 1..1491: mol_type = other DNA
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagataggtggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaatgagcctagacgattggaagcccagtgaagacccttgggaacaacatgctaaatggtatagcgggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtct

SEQ ID NO: 113 - protein, 497 aa, synthetic construct
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLSDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 114 - DNA, 1491 bp, synthetic construct
  source 1..1491: mol_type = other DNA
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagataggtggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaagcgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtct

SEQ ID NO: 115 - protein, 497 aa, synthetic construct
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLDDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 116 - DNA, 1491 bp, synthetic construct
  source 1..1491: mol_type = other DNA
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagataggtggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctagacgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtct

SEQ ID NO: 117 - protein, 497 aa, synthetic construct
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGSLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 118 - DNA, 1491 bp, synthetic construct
  source 1..1491: mol_type = other DNA
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagataggtggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggaagcctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtct

SEQ ID NO: 119 - protein, 497 aa, synthetic construct
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGMGLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 120 - DNA, 1491 bp, synthetic construct
  source 1..1491: mol_type = other DNA
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagataggtggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaatggggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtct

SEQ ID NO: 121 - protein, 497 aa, synthetic construct
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLTDWKPSEDPWEQHAKWYSGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 122 - DNA, 1491 bp, synthetic construct
  source 1..1491: mol_type = other DNA
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagataggtggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatagcgggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtct

SEQ ID NO: 123 - protein, 422 aa, synthetic construct
MDEADRRLLRRCRLRLVEELQVDQLWDVLLSRELFRPHMIEDIQRAGSGSRRDQARQLIIDLETRGSQALPLFISCLEDTGQDMLASFLRTNRQAAKLSKPTLENLTPVVLRPEIRKPEVLRPETPRPVDIGSGGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSHHHHHH

SEQ ID NO: 124 - DNA, 1266 bp, synthetic construct
  source 1..1266: mol_type = other DNA
atggacgaagcggatcggcggctcctgcggcggtgccggctgcggctggtggaagagctgcaggtggaccagctctgggacgtcctgctgagccgcgagctgttcaggccccatatgatcgaggacatccagcgggcaggctctggatctcggcgggatcaggccaggcagctgatcatagatctggagactcgagggagtcaggctcttcctttgttcatctcctgcttagaggacacaggccaggacatgctggcttcgtttctgcgaactaacaggcaagcagcaaagttgtcgaagccaaccctagaaaaccttaccccagtggtgctcagaccagagattcgcaaaccagaggttctcagaccggaaacacccagaccagtggacattggttctggaggattcggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcacaccaccaccaccaccac

SEQ ID NO: 125 - protein, 280 aa, synthetic construct
MDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 126 - DNA, 840 bp, synthetic construct
  source 1..840: mol_type = other DNA
atggatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 127-134 - skipped (8 entries)

SEQ ID NO: 135 - protein, 239 aa, synthetic construct
MAALKSWLSRSVTSFFRYRQCLCVPVVANFKKRCFSELIRPWHKTVTIGFGVTLCAVPIAQKSEPHSLSSEALMRRAVSLVTDSTSTFLSQTTYALIEAITEYTKAVYTLTSLYRQYTSLLGKMNSEEEDEVWQVIIGARAEMTSKHQEYLKLETTWMTAVGLSEMAAEAAYQTGADQASITARNHIQLVKLQVEEVHQLSRKAETKLAEAQIEELRQKTQEEGEERAESEQEAYLRED

SEQ ID NO: 136 - DNA, 717 bp, synthetic construct
  source 1..717: mol_type = other DNA
atggccgctctgaagtcctggctgagcagaagcgtgaccagcttcttccggtacagacagtgcctgtgcgtgcccgtggtggccaacttcaagaagagatgcttcagcgagctgatcagaccctggcacaagaccgtgaccatcggctttggcgtgaccctgtgtgccgtgcctatcgctcagaagtctgagcctcacagcctgtctagcgaggcccttatgagaagggccgtgtctctggtcaccgacagcaccagcacatttctgagccagaccacatacgccctgatcgaggccatcaccgagtacaccaaggccgtgtacaccctgaccagcctgtaccggcagtacacatctctgctgggcaagatgaacagcgaggaagaggacgaagtctggcaagtgatcatcggcgccagagccgagatgaccagcaagcaccaagagtacctgaagctggaaaccacctggatgacagccgtgggcctgtctgaaatggccgccgaagctgcttatcagaccggcgctgatcaggccagcatcaccgccagaaatcacatccagctggtcaagctgcaggtcgaggaagtgcaccagctgtccagaaaggccgagacaaagctggctgaggcccagatcgaggaactgcggcagaaaacccaagaggaaggcgaggaaagagccgagtctgagcaagaggcctacctgagagaggac

SEQ ID NO: 137-152 - skipped (16 entries)

SEQ ID NO: 153 - DNA, 582 bp, synthetic construct
  source 1..582: mol_type = other DNA
gactgtgaggtcaacaacggttccagcctcagggatgagtgcatcacaaacctactggtgtttggcttcctccaaagctgttctgacaacagcttccgcagagagctggacgcactgggccacgagctgccagtgctggctccccagtgggagggctacgatgagctgcagactgatggcaaccgcagcagccactcccgcttgggaagaatagaggcagattctgaaagtcaagaagacatcatccggaatattgccaggcacctcgcccaggtcggggacagcatggaccgtagcatccctccgggcctggtgaacggcctggccctgcagctcaggaacaccagccggtcggaggaggaccggaacagggacctggccactgccctggagcagctgctgcaggcctaccctagagacatggagaaggagaagaccatgctggtgctggccctgctgctggccaagaaggtggccagtcacacgccgtccttgctccgtgatgtctttcacacaacagtgaattttattaaccagaacctacgcacctacgtgaggagcttagccagaaatgggatggac

SEQ ID NO: 154 - skipped

SEQ ID NO: 155 - protein, 6 aa, synthetic construct
HHHHHH

SEQ ID NO: 156 - protein, 4 aa, synthetic construct
WRPW

SEQ ID NO: 157 - DNA, 532 bp, synthetic construct
  source 1..532: mol_type = other DNA
agtgcggttaccagcggaaatgcctcggggtcagaagtcgcaggagagatagacagctgctgaaccaatgggaccagcggatggggcggatgttatctaccattggtgaacgttagaaacgaatagcagccaatgaatcagctgggggggcggagcagtgacgtttattgcggagggggccgcttcgaatcggcggcggccagcttggtggcctgggccaatgaacggcctccaacgagcagggccttcaccaatcggcggcctccacgacggggctgggggagggtatataagccgagtaggcgacggtgaggtcgacgccggccaagacagcacagacagattgacctattggggtgtttcgcgagtgtgagagggaagcgccgcggcctgtatttctagacctgcccttcgcctggttcgtggcgccttgtgaccccgggcccctgccgcctgcaagtcggaaattgcgctgtgctcctgtgctacggcctgtggctggactgcctgctgctgcccaactggctggcac

SEQ ID NO: 158 - protein, 315 aa, synthetic construct
MSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGVVWRSMEHPVKLLFAPNLVLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPATA

SEQ ID NO: 159 - protein, 315 aa, synthetic construct
MSAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGLVWRSMEHPVKLLFAPNLLLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATA

SEQ ID NO: 160 - protein, 315 aa, synthetic construct
MSAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLVLDRDEGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATA

SEQ ID NO: 161 - protein, 315 aa, synthetic construct
MSAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWMEILMIGVVWRSMEHPVKLLFAPNLVLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATA

SEQ ID NO: 162 - protein, 315 aa, synthetic construct
MSAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLLLDRDEGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPATA

SEQ ID NO: 163 - protein, 315 aa, synthetic construct
MSAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLLLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPATA

SEQ ID NO: 164 - protein, 315 aa, synthetic construct
MSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGVVWRSMEHPVKLLFAPNLLLDRDQGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPATA

SEQ ID NO: 165 - protein, 315 aa, synthetic construct
MSAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWMEILMIGLVWRSMEHPVKLLFAPNLLLDRDQGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATA

SEQ ID NO: 166 - protein, 315 aa, synthetic construct
MSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGVVWRSMEHPVKLLFAPNLVLDRDQGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPATA

SEQ ID NO: 167 - protein, 315 aa, synthetic construct
MSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGVVWRSMEHPVKLLFAPNLLLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATA

SEQ ID NO: 168 - protein, 313 aa, synthetic construct
AGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATA

SEQ ID NO: 169 - DNA, 671 bp, synthetic construct
  source 1..671: mol_type = other DNA
tctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctgtttcgcctcggcgtccggtccgtgttgcttggtcgtcacctgtgcagaattgcgaaccatggattcc

SEQ ID NO: 170 - protein, 312 aa, synthetic construct
AGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGLVWRSMEHPVKLLFAPNLVLDRDQGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPAT

SEQ ID NO: 171 - protein, 312 aa, synthetic construct
AGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGVVWRSMEHPVKLLFAPNLLLDRDEGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMEFLYSMKCKNVVPLYDLLLEAADAHRLHAPTSRGGASVEETDQSHLATAGSTSSHSLQKYYITGEAEGFPAT

SEQ ID NO: 172 - protein, 312 aa, synthetic construct
AGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLLLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPAT

SEQ ID NO: 173 - protein, 312 aa, synthetic construct
AGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGVVWRSMEHPVKLLFAPNLLLDRNEGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMEFLYSMKCKNVVPLYDLLLEAADAHRLHAPTSRGGASVEETDQSHLATAGSTSSHSLQKYYITGEAEGFPAT

SEQ ID NO: 174 - protein, 312 aa, synthetic construct
AGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGLVWRSMEHPVKLLFAPNLVLDRDQGKCVEGLVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPAT

SEQ ID NO: 175 - protein, 312 aa, synthetic construct
AGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGVVWRSMEHPVKLLFAPNLVLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMEFLYSMKCKNVVPLYDLLLEAADAHRLHAPTSRGGASVEETDQSHLATAGSTSSHSLQKYYITGEAEGFPAT

SEQ ID NO: 176 - protein, 311 aa, synthetic construct
AGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGVVWRSMEHPVKLLFAPNLLLDRDQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEFLYSMKCKNVVPLYDLLLEAADAHRLHAPTSRGGASVEETDQSHLATAGSTSSHSLQKYYITGEAEGFPA

SEQ ID NO: 177 - DNA, 243 bp, synthetic construct
  source 1..243: mol_type = other DNA
cgggtttcgtaacaatcgcatgaggattcgcaacgcctttgaagcagtcgacgccgaagtcccgtctcagtaaaggttgaagcagtcgacgccgaagaatcggactgccttcgtatgaagcagtcgacgccgaaggtatcagtcgcctcggaatgaagcagtcgacgccgaagattcgtaagaggctcactctcccttacacggagtggataactagttctagagggtatataatgggggcca

SEQ ID NO: 178 - DNA, 1623 bp, synthetic construct
  source 1..1623: mol_type = other DNA
atgtgccatcagcaactcgtcatctcctggttctcccttgtgttcctcgcttcccctctggtcgccatttgggaactgaagaaggacgtctacgtggtcgagctggattggtacccggacgcccctggagaaatggtcgtgctgacttgcgatacgccagaagaggacggcataacctggaccctggatcagagctccgaggtgctcggaagcggaaagaccctgaccattcaagtcaaggagttcggcgacgcgggccagtacacttgccacaagggtggcgaagtgctgtcccactccctgctgctgctgcacaagaaagaggatggaatctggtccactgacatcctcaaggaccaaaaagaaccgaagaacaagaccttcctccgctgcgaagccaagaactacagcggtcggttcacctgttggtggctgacgacaatctccaccgacctgactttctccgtgaagtcgtcacggggatcaagcgatcctcagggcgtgacctgtggagccgccactctgtccgccgagagagtcaggggagacaacaaggaatatgagtactccgtggaatgccaggaggacagcgcctgccctgccgcggaagagtccctgcctatcgaggtcatggtcgatgccgtgcataagctgaaatacgagaactacacttcctccttctttatccgcgacatcatcaagcctgacccccccaagaacttgcagctgaagccactcaagaactcccgccaagtggaagtgtcttgggaatatccagacacttggagcaccccgcactcatacttctcgctcactttctgtgtgcaagtgcagggaaagtccaaacgggagaagaaagaccgggtgttcaccgacaaaacctccgccactgtgatttgtcggaagaacgcgtcaatcagcgtccgggcgcaggatagatactactcgtcctcctggagcgaatgggccagcgtgccttgttccggtggcggatcaggcggaggttcaggaggaggctccggaggaggttcccggaacctccctgtggcaacccccgaccctggaatgttcccgtgcctacaccactcccaaaacctcctgagggctgtgtcgaacatgttgcagaaggcccgccagacccttgagttctacccctgcacctcggaagaaattgatcacgaggacatcaccaaggacaagacctcgaccgtggaagcctgcctgccgctggaactgaccaagaacgaatcgtgtctgaactcccgcgagacaagctttatcactaacggcagctgcctggcgtcgagaaagacctcattcatgatggcgctctgtctttcctcgatctacgaagatctgaagatgtatcaggtcgagttcaagaccatgaacgccaagctgctcatggacccgaagcggcagatcttcctggaccagaatatgctcgccgtgattgatgaactgatgcaggccctgaatttcaactccgagactgtgcctcaaaagtccagcctggaagaaccggacttctacaagaccaagatcaagctgtgcatcctgttgcacgctttccgcattcgagccgtgaccattgaccgcgtgatgtcctacctgaacgccagt

SEQ ID NO: 179 - DNA, 51 bp, synthetic construct
  source 1..51: mol_type = other DNA
atttatttatttatttatttaacatcggttccctgtttaatatttaaacag

SEQ ID NO: 180 - DNA, 266 bp, synthetic construct
  source 1..266: mol_type = other DNA
agagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgt

SEQ ID NO: 181 - DNA, 11809 bp, synthetic construct
  source 1..11809: mol_type = other DNA
aagcttgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgagtccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttacttactgtttaaatattaaacagggaaccgatgttaaataaataaataaataaatgtttaaactagagtcgcggcctcagtcagtcacgcatgcctgcagtttaactggcgttcaggtaggacatcacgcggtcaatggtcacggctcgaatgcggaaagcgtgcaacaggatgcacagcttgatcttggtcttgtagaagtccggttcttccaggctggacttttgaggcacagtctcggagttgaaattcagggcctgcatcagttcatcaatcacggcgagcatattctggtccaggaagatctgccgcttcgggtccatgagcagcttggcgttcatggtcttgaactcgacctgatacatcttcagatcttcgtagatcgaggaaagacagagcgccatcatgaatgaggtctttctcgacgccaggcagctgccgttagtgataaagcttgtctcgcgggagttcagacacgattcgttcttggtcagttccagcggcaggcaggcttccacggtcgaggtcttgtccttggtgatgtcctcgtgatcaatttcttccgaggtgcaggggtagaactcaagggtctggcgggccttctgcaacatgttcgacacagccctcaggaggttttgggagtggtgtaggcacgggaacattccagggtcgggggttgccacagggaggttccgggaacctcctccggagcctcctcctgaacctccgcctgatccgccaccggaacaaggcacgctggcccattcgctccaggaggacgagtagtatctatcctgcgcccggacgctgattgacgcgttcttccgacaaatcacagtggcggaggttttgtcggtgaacacccggtctttcttctcccgtttggactttccctgcacttgcacacagaaagtgagcgagaagtatgagtgcggggtgctccaagtgtctggatattcccaagacacttccacttggcgggagttcttgagtggcttcagctgcaagttcttgggggggtcaggcttgatgatgtcgcggataaagaaggaggaagtgtagttctcgtatttcagcttatgcacggcatcgaccatgacctcgataggcagggactcttccgcggcagggcaggcgctgtcctcctggcattccacggagtactcatattccttgttgtctcccctgactctctcggcggacagagtggcggctccacaggtcacgccctgaggatcgcttgatccccgtgacgacttcacggagaaagtcaggtcggtggagattgtcgtcagccaccaacaggtgaaccgaccgctgtagttcttggcttcgcagcggaggaaggtcttgttcttcggttctttttggtccttgaggatgtcagtggaccagattccatcctctttcttgtgcagcagcagcagggagtgggacagcacttcgccacccttgtggcaagtgtactggcccgcgtcgccgaactccttgacttgaatggtcagggtctttccgcttccgagcacctcggagctctgatccagggtccaggttatgccgtcctcttctggcgtatcgcaagtcagcacgaccatttctccaggggcgtccgggtaccaatccagctcgaccacgtagacgtccttcttcagttcccaaatggcgaccagaggggaagcgaggaacacaagggagaaccaggagatgacgagttgctgatggcacatcatggtggcgacaccggtacgcgttggcccccattatataccctctagaactagttatccactccgtgtaagggagagtgagcctcttacgaatcttcggcgtcgactgcttcattccgaggcgactgataccttcggcgtcgactgcttcatacgaaggcagtccgattcttcggcgtcgactgcttcaacctttactgagacgggacttcggcgtcgactgcttcaaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgtccatctagatggccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggatccgccaccatgtctagacctggcgagaggcccttccagtgccggatctgcatgcggaacttcagcaacatgagcaacctgaccagacacacccggacacacacaggcgagaagccttttcagtgcagaatctgtatgcgcaatttctccgacagaagcgtgctgcggagacacctgagaacccacaccggcagccagaaaccattccagtgtcgcatctgtatgagaaactttagcgacccctccaatctggcccggcacaccagaacacataccggggaaaaaccctttcagtgtaggatatgcatgaggaatttttccgaccggtccagcctgaggcggcacctgaggacacatactggctcccaaaagccgttccaatgtcggatatgtatgcgcaactttagccagagcggcaccctgcacagacacacaagaacccatactggcgagaaacctttccaatgtagaatctgcatgcgaaatttttcccagcggcctaatctgaccaggcatctgaggacccacctgagaggatctacctgcagggatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcccaattgtgcgtacgcggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggatggagatcctgatgattggtgtggtctggcgctccatggagcacccagtgaagctactgtttgctcctaacttgctcttggacagggaccagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacataagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctacgccttcgccctcagacgagtcggatctccctttgggccgcctccccgcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 182 - DNA, 11809 bp, synthetic construct
  source 1..11809: mol_type = other DNA
aagcttgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgagtccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttacttactgtttaaatattaaacagggaaccgatgttaaataaataaataaataaatgtttaaactagagtcgcggcctcagtcagtcacgcatgcctgcagtttaactggcgttcaggtaggacatcacgcggtcaatggtcacggctcgaatgcggaaagcgtgcaacaggatgcacagcttgatcttggtcttgtagaagtccggttcttccaggctggacttttgaggcacagtctcggagttgaaattcagggcctgcatcagttcatcaatcacggcgagcatattctggtccaggaagatctgccgcttcgggtccatgagcagcttggcgttcatggtcttgaactcgacctgatacatcttcagatcttcgtagatcgaggaaagacagagcgccatcatgaatgaggtctttctcgacgccaggcagctgccgttagtgataaagcttgtctcgcgggagttcagacacgattcgttcttggtcagttccagcggcaggcaggcttccacggtcgaggtcttgtccttggtgatgtcctcgtgatcaatttcttccgaggtgcaggggtagaactcaagggtctggcgggccttctgcaacatgttcgacacagccctcaggaggttttgggagtggtgtaggcacgggaacattccagggtcgggggttgccacagggaggttccgggaacctcctccggagcctcctcctgaacctccgcctgatccgccaccggaacaaggcacgctggcccattcgctccaggaggacgagtagtatctatcctgcgcccggacgctgattgacgcgttcttccgacaaatcacagtggcggaggttttgtcggtgaacacccggtctttcttctcccgtttggactttccctgcacttgcacacagaaagtgagcgagaagtatgagtgcggggtgctccaagtgtctggatattcccaagacacttccacttggcgggagttcttgagtggcttcagctgcaagttcttgggggggtcaggcttgatgatgtcgcggataaagaaggaggaagtgtagttctcgtatttcagcttatgcacggcatcgaccatgacctcgataggcagggactcttccgcggcagggcaggcgctgtcctcctggcattccacggagtactcatattccttgttgtctcccctgactctctcggcggacagagtggcggctccacaggtcacgccctgaggatcgcttgatccccgtgacgacttcacggagaaagtcaggtcggtggagattgtcgtcagccaccaacaggtgaaccgaccgctgtagttcttggcttcgcagcggaggaaggtcttgttcttcggttctttttggtccttgaggatgtcagtggaccagattccatcctctttcttgtgcagcagcagcagggagtgggacagcacttcgccacccttgtggcaagtgtactggcccgcgtcgccgaactccttgacttgaatggtcagggtctttccgcttccgagcacctcggagctctgatccagggtccaggttatgccgtcctcttctggcgtatcgcaagtcagcacgaccatttctccaggggcgtccgggtaccaatccagctcgaccacgtagacgtccttcttcagttcccaaatggcgaccagaggggaagcgaggaacacaagggagaaccaggagatgacgagttgctgatggcacatcatggtggcgacaccggtacgcgttggcccccattatataccctctagaactagttatccactccgtgtaagggagagtgagcctcttacgaatcttcggcgtcgactgcttcattccgaggcgactgataccttcggcgtcgactgcttcatacgaaggcagtccgattcttcggcgtcgactgcttcaacctttactgagacgggacttcggcgtcgactgcttcaaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgtccatctagatggccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggatccgccaccatgtctagacctggcgagaggcccttccagtgccggatctgcatgcggaacttcagcaacatgagcaacctgaccagacacacccggacacacacaggcgagaagccttttcagtgcagaatctgtatgcgcaatttctccgacagaagcgtgctgcggagacacctgagaacccacaccggcagccagaaaccattccagtgtcgcatctgtatgagaaactttagcgacccctccaatctggcccggcacaccagaacacataccggggaaaaaccctttcagtgtaggatatgcatgaggaatttttccgaccggtccagcctgaggcggcacctgaggacacatactggctcccaaaagccgttccaatgtcggatatgtatgcgcaactttagccagagcggcaccctgcacagacacacaagaacccatactggcgagaaacctttccaatgtagaatctgcatgcgaaatttttcccagcggcctaatctgaccaggcatctgaggacccacctgagaggatctacctgcagggatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcccaattgtgcgtacgcggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagatcgttcacatgatcaactgggcgaagagggtgccaggctttgtggatttgaccctccatgatcaggtccaccttctagaatgtgcctggctagagatcctgatgattggtgtggtctggcgctccatggagcacccagtgaagctactgtttgctcctaacttgctcttggacagggaccagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacgccagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacataagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctacgccttcgccctcagacgagtcggatctccctttgggccgcctccccgcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 183 - DNA, 11809 bp, synthetic construct
  source 1..11809: mol_type = other DNA
aagcttgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgagtccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttacttactgtttaaatattaaacagggaaccgatgttaaataaataaataaataaatgtttaaactagagtcgcggcctcagtcagtcacgcatgcctgcagtttaactggcgttcaggtaggacatcacgcggtcaatggtcacggctcgaatgcggaaagcgtgcaacaggatgcacagcttgatcttggtcttgtagaagtccggttcttccaggctggacttttgaggcacagtctcggagttgaaattcagggcctgcatcagttcatcaatcacggcgagcatattctggtccaggaagatctgccgcttcgggtccatgagcagcttggcgttcatggtcttgaactcgacctgatacatcttcagatcttcgtagatcgaggaaagacagagcgccatcatgaatgaggtctttctcgacgccaggcagctgccgttagtgataaagcttgtctcgcgggagttcagacacgattcgttcttggtcagttccagcggcaggcaggcttccacggtcgaggtcttgtccttggtgatgtcctcgtgatcaatttcttccgaggtgcaggggtagaactcaagggtctggcgggccttctgcaacatgttcgacacagccctcaggaggttttgggagtggtgtaggcacgggaacattccagggtcgggggttgccacagggaggttccgggaacctcctccggagcctcctcctgaacctccgcctgatccgccaccggaacaaggcacgctggcccattcgctccaggaggacgagtagtatctatcctgcgcccggacgctgattgacgcgttcttccgacaaatcacagtggcggaggttttgtcggtgaacacccggtctttcttctcccgtttggactttccctgcacttgcacacagaaagtgagcgagaagtatgagtgcggggtgctccaagtgtctggatattcccaagacacttccacttggcgggagttcttgagtggcttcagctgcaagttcttgggggggtcaggcttgatgatgtcgcggataaagaaggaggaagtgtagttctcgtatttcagcttatgcacggcatcgaccatgacctcgataggcagggactcttccgcggcagggcaggcgctgtcctcctggcattccacggagtactcatattccttgttgtctcccctgactctctcggcggacagagtggcggctccacaggtcacgccctgaggatcgcttgatccccgtgacgacttcacggagaaagtcaggtcggtggagattgtcgtcagccaccaacaggtgaaccgaccgctgtagttcttggcttcgcagcggaggaaggtcttgttcttcggttctttttggtccttgaggatgtcagtggaccagattccatcctctttcttgtgcagcagcagcagggagtgggacagcacttcgccacccttgtggcaagtgtactggcccgcgtcgccgaactccttgacttgaatggtcagggtctttccgcttccgagcacctcggagctctgatccagggtccaggttatgccgtcctcttctggcgtatcgcaagtcagcacgaccatttctccaggggcgtccgggtaccaatccagctcgaccacgtagacgtccttcttcagttcccaaatggcgaccagaggggaagcgaggaacacaagggagaaccaggagatgacgagttgctgatggcacatcatggtggcgacaccggtacgcgttggcccccattatataccctctagaactagttatccactccgtgtaagggagagtgagcctcttacgaatcttcggcgtcgactgcttcattccgaggcgactgataccttcggcgtcgactgcttcatacgaaggcagtccgattcttcggcgtcgactgcttcaacctttactgagacgggacttcggcgtcgactgcttcaaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgtccatctagatggccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggatccgccaccatgtctagacctggcgagaggcccttccagtgccggatctgcatgcggaacttcagcaacatgagcaacctgaccagacacacccggacacacacaggcgagaagccttttcagtgcagaatctgtatgcgcaatttctccgacagaagcgtgctgcggagacacctgagaacccacaccggcagccagaaaccattccagtgtcgcatctgtatgagaaactttagcgacccctccaatctggcccggcacaccagaacacataccggggaaaaaccctttcagtgtaggatatgcatgaggaatttttccgaccggtccagcctgaggcggcacctgaggacacatactggctcccaaaagccgttccaatgtcggatatgtatgcgcaactttagccagagcggcaccctgcacagacacacaagaacccatactggcgagaaacctttccaatgtagaatctgcatgcgaaatttttcccagcggcctaatctgaccaggcatctgaggacccacctgagaggatctacctgcagggatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcccaattgtgcgtacgcggatcctctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttggtgttggacagggacgagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacataagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctacgccttcgccctcagacgagtcggatctccctttgggccgcctccccgcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 184 - DNA, 1623 bp, synthetic construct
  source 1..1623: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggcggaggaagcggtggcggatcaggtggtggatctggcggcggatctagaaacctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctct

SEQ ID NO: 185 - DNA, 886 bp, synthetic construct
  source 1..886: mol_type = other DNA
atggctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggatggagatcctgatgattggtgtggtctggcgctccatggagcacccagtgaagctactgtttgctcctaacttgctcttggacagggaccagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgc

SEQ ID NO: 186 - DNA, 2789 bp, synthetic construct
  source 1..2789: mol_type = other DNA
atggctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtgtggtctggcgctccatggagcacccagtgaagctactgtttgctcctaacttgctcttggacagggacgagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagtttctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagggggtggaggttcagggggtggaggttcaggtggtggcggtagtgtcgatggcttcgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcatagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatggccacagaattcatggtgagcaagggcgaggagctgatcaaggagaacatgcacagcaagctgtacctggaaggcagcgtgaacggccaccagttcaagtgcacccacgaaggggagggcaagccctacgagggcacccagaccaacaggatcaaggtggtggagggaggccccctgccgttcgcattcgacatcctggccaccatgtttatgtacgggagcaaggccttcatcaagtaccccaagggcctccccgattattttaagcagtccttccctgagggcttcacatgggagagagtcatggtgttcgaagacgggggcgtgctgaccgccacccaggacaccagcctccaggacggctgcctcatctacaacgtgaagctgagaggggtgaacttcccagccaacggccccgtgatgaagcagacaacactgggctgggagcccagcaccgagaccctgtaccccgctgacggcgccctggaaggcagatgcgacatggccctgaagctcgtgggcgggggccacctgcactgcaacttcaagaccacatacaaatccaagaaacccgctacaaacctcaagatgcccggcgtccactacgtggaccgcagactggaaagaatcaaggaggccgacaacgagacctacgtcgagcagcacgaggtggctgtggccagatactgcgacctccctagcaaactggggcacaaacttaatggcatggacgagctgtacaagtga

SEQ ID NO: 187 - DNA, 2789 bp, synthetic construct
  source 1..2789: mol_type = other DNA
atggctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagatcgttcacatgatcaactgggcgaagagggtgccaggctttgtggatttgaccctccatgatcaggtccaccttctagaatgtgcctggctagagatcctgatgattggtgtggtctggcgctccatggagcacccagtgaagctactgtttgctcctaacttgctcttggacagggacgagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacgccagtaacaaaggcatggagtttctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagggggtggaggttcagggggtggaggttcaggtggtggcggtagtgtcgatggcttcgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcatagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatggccacagaattcatggtgagcaagggcgaggagctgatcaaggagaacatgcacagcaagctgtacctggaaggcagcgtgaacggccaccagttcaagtgcacccacgaaggggagggcaagccctacgagggcacccagaccaacaggatcaaggtggtggagggaggccccctgccgttcgcattcgacatcctggccaccatgtttatgtacgggagcaaggccttcatcaagtaccccaagggcctccccgattattttaagcagtccttccctgagggcttcacatgggagagagtcatggtgttcgaagacgggggcgtgctgaccgccacccaggacaccagcctccaggacggctgcctcatctacaacgtgaagctgagaggggtgaacttcccagccaacggccccgtgatgaagcagacaacactgggctgggagcccagcaccgagaccctgtaccccgctgacggcgccctggaaggcagatgcgacatggccctgaagctcgtgggcgggggccacctgcactgcaacttcaagaccacatacaaatccaagaaacccgctacaaacctcaagatgcccggcgtccactacgtggaccgcagactggaaagaatcaaggaggccgacaacgagacctacgtcgagcagcacgaggtggctgtggccagatactgcgacctccctagcaaactggggcacaaacttaatggcatggacgagctgtacaagtga

SEQ ID NO: 188 - DNA, 2789 bp, synthetic construct
  source 1..2789: mol_type = other DNA
atggctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagatcgttcacatgatcaactgggcgaagagggtgccaggctttgtggatttgaccctccatgatcaggtccaccttctagaatgtgcctggctagagatcctgatgattggtgtggtctggcgctccatggagcacccagtgaagctactgtttgctcctaacttggtgttggacagggacgagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagggggtggaggttcagggggtggaggttcaggtggtggcggtagtgtcgatggcttcgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcatagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatggccacagaattcatggtgagcaagggcgaggagctgatcaaggagaacatgcacagcaagctgtacctggaaggcagcgtgaacggccaccagttcaagtgcacccacgaaggggagggcaagccctacgagggcacccagaccaacaggatcaaggtggtggagggaggccccctgccgttcgcattcgacatcctggccaccatgtttatgtacgggagcaaggccttcatcaagtaccccaagggcctccccgattattttaagcagtccttccctgagggcttcacatgggagagagtcatggtgttcgaagacgggggcgtgctgaccgccacccaggacaccagcctccaggacggctgcctcatctacaacgtgaagctgagaggggtgaacttcccagccaacggccccgtgatgaagcagacaacactgggctgggagcccagcaccgagaccctgtaccccgctgacggcgccctggaaggcagatgcgacatggccctgaagctcgtgggcgggggccacctgcactgcaacttcaagaccacatacaaatccaagaaacccgctacaaacctcaagatgcccggcgtccactacgtggaccgcagactggaaagaatcaaggaggccgacaacgagacctacgtcgagcagcacgaggtggctgtggccagatactgcgacctccctagcaaactggggcacaaacttaatggcatggacgagctgtacaagtga

SEQ ID NO: 189 - DNA, 1833 bp, synthetic construct
  source 1..1833: mol_type = other DNA
atggctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggatggagatcctgatgattggtgtggtctggcgctccatggagcacccagtgaagctactgtttgctcctaacttgctcttggacagggaccagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagggggtggaggttcagggggtggaggttcaggtggtggcggtagtgtcgatggcttcgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 190 - DNA, 1833 bp, synthetic construct
  source 1..1833: mol_type = other DNA
atggctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtgtggtctggcgctccatggagcacccagtgaagctactgtttgctcctaacttgctcttggacagggacgagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagtttctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagggggtggaggttcagggggtggaggttcaggtggtggcggtagtgtcgatggcttcgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 191 - DNA, 1833 bp, synthetic construct
  source 1..1833: mol_type = other DNA
atggctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagatcgttcacatgatcaactgggcgaagagggtgccaggctttgtggatttgaccctccatgatcaggtccaccttctagaatgtgcctggctagagatcctgatgattggtgtggtctggcgctccatggagcacccagtgaagctactgtttgctcctaacttgctcttggacagggacgagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacgccagtaacaaaggcatggagtttctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagggggtggaggttcagggggtggaggttcaggtggtggcggtagtgtcgatggcttcgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 192 - DNA, 1833 bp, synthetic construct
  source 1..1833: mol_type = other DNA
atggctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagatcgttcacatgatcaactgggcgaagagggtgccaggctttgtggatttgaccctccatgatcaggtccaccttctagaatgtgcctggctagagatcctgatgattggtgtggtctggcgctccatggagcacccagtgaagctactgtttgctcctaacttggtgttggacagggacgagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagggggtggaggttcagggggtggaggttcaggtggtggcggtagtgtcgatggcttcgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 193 - protein, 611 aa, synthetic construct
AGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGVVWRSMEHPVKLLFAPNLLLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATGGGGSGGGGSGGGGSVDGFMDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 194 - protein, 611 aa, synthetic construct
AGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGVVWRSMEHPVKLLFAPNLLLDRDEGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMEFLYSMKCKNVVPLYDLLLEAADAHRLHAPTSRGGASVEETDQSHLATAGSTSSHSLQKYYITGEAEGFPATGGGGSGGGGSGGGGSVDGFMDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 195 - protein, 611 aa, synthetic construct
AGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLLLDRDEGKCVEGLVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHASNKGMEFLYSMKCKNVVPLYDLLLEAADAHRLHAPTSRGGASVEETDQSHLATAGSTSSHSLQKYYITGEAEGFPATGGGGSGGGGSGGGGSVDGFMDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 196 - protein, 611 aa, synthetic construct
AGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLVLDRDEGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATGGGGSGGGGSGGGGSVDGFMDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

(19) United States
(12) Patent Application Publication
(10) Pub. No.: US 2026/0022364 A1
(43) Pub. Date: Jan. 22, 2026

(54) ERT2 MUTANTS, INDUCIBLE CELL DEATH SYSTEMS, AND USES THEREOF

(71) Applicant: Senti Biosciences, Inc., South San Francisco, CA (US)

(72) Inventors: Rebecca Tayler Cottman, South San Francisco, CA (US); Michelle Elizabeth Hung, South San Francisco, CA (US); Russell Morrison Gordley, South San Francisco, CA (US); Timothy Kuan-Ta Lu, South San Francisco, CA (US); Assen Boyanov Roguev, South San Francisco, CA (US); Karen Lai Len Chu, South San Francisco, CA (US)

(21) Appl. No.: 19/288,667

(22) Filed: Aug. 1, 2025

Related U.S. Application Data
(63) Continuation of application No. PCT/US2024/014268, filed on Feb. 2, 2024.
(60) Provisional application No. 63/482,983, filed on Feb. 2, 2023.
(60) Provisional application No. 63/466,147, filed on May 12, 2023.

(51) Int. Cl. C12N 9/64; C07K 14/72; C12N 15/85

(52) U.S. Cl. CPC C12N 9/641; C07K 14/72; C12N 15/85; C07K 2319/09; C07K 2319/715; C12Y 304/22062

(57) ABSTRACT

Provided herein are mutants of estrogen receptor alpha ligand binding domain (ER-LBD), and inducible cell death systems that include mutants of estrogen receptor alpha ligand binding domain (ER-LBD). Also provided are methods of for use of the same, such as inducing cell death in a cell.

### CROSS-REFERENCE TO RELATED APPLICATIONS

This application is a continuation application of International Application No. PCT/US2024/014268 filed Feb. 2, 2024, now pending; which claims the benefit under 35 USC § 119 (c) to U.S. Application Ser. No. 63/482,983 filed Feb. 2, 2023 and to U.S. Application Ser. No. 63/466,147 filed May 12, 2023, both now expired. The disclosure of each of the prior applications is considered part of and is incorporated by reference in the disclosure of this application.

### INCORPORATION BY REFERENCE OF SEQUENCE LISTING

The material in the accompanying sequence listing is hereby incorporated by reference into this application. The accompanying sequence listing xml file, named SENTI1400-2_ST26.xml was created on Jul. 27, 2025 and is 316,641 bytes in size.

### BACKGROUND OF THE INVENTION

Estrogen receptor (ER) is a ligand-dependent transcription factor that binds endogenous hormone ligands such as estrogen and estradiol. Synthetic ligands that bind to ER have been developed for treating ER-positive cancers such as ER-positive breast cancer. For example, active metabolites of the drug tamoxifen induce nuclear translocation of ER and antagonize ER in a tissue-selective manner. Tamoxifen and its active metabolites are also utilized as a tool for controlling nuclear localization in the research setting. For example, an ER ligand binding domain variant known as ERT2 has been used as a fusion protein with Cre recombinase to regulate Cre recombinase-based gene editing in animal model systems. The ability to manipulate suicide-switch mediated cells killing using a synthetic ligand would also be useful in therapeutic applications, such as in the field of cell and gene therapy. For example, it can be desirable to engineer cell and gene therapy products with a “safety” switch, e.g., an inducible cell death system, to address potential toxicity concerns. Thus, modified ERT2-based systems with improved sensitivity to and/or selectivity for synthetic ligands would be useful for suicide-switch mediated regulated cell killing in a clinical setting.

### SUMMARY OF THE INVENTION

Provided herein, in various embodiments, is an inducible cell-death system comprising a polypeptide, wherein the polypeptide comprises a ligand binding domain and a cell death inducing domain, wherein the polypeptide is configured upon contact with a ligand of the ligand binding domain to generate a cell-death inducing signal in a cell in which the polypeptide is expressed, and wherein the ligand binding domain comprises a modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions are with reference to one or more regions selected from: positions 343-354, positions 380-392, positions 404-463, positions 517-540, and position 547 of SEQ ID NO: 1, optionally wherein the modified ER-LBD has greater sensitivity to a non-endogenous ligand as compared to an ER-LBD comprising the amino acid sequence of SEQ ID NO: 2, optionally wherein the modified ER-LBD has greater sensitivity to a non-endogenous ligand as compared to an endogenous ligand as a result of the one or more additional amino acid substitutions, optionally wherein the modified ER-LBD has greater selectivity to a non-endogenous ligand as compared to an ER-LBD comprising the amino acid sequence of SEQ ID NO: 2, optionally wherein the ligand binding domain of the first polypeptide monomer and the second polypeptide monomer comprise the same additional amino acid substitutions.

In some embodiments, (a) the one or more additional amino acid substitutions of the polypeptide, the first polypeptide monomer, and/or the second polypeptide monomer comprise an L391 substitution, optionally wherein the L391 substitution is L391V; (b) the one or more additional amino acid substitutions of the polypeptide, the first polypeptide monomer, and/or the second polypeptide monomer comprise an N413D mutation; (c) the one or more additional amino acid substitutions of the polypeptide, the first polypeptide monomer, and/or the second polypeptide monomer comprise an L391V substitution and an N413D mutation; (d) the one or more additional amino acid substitutions of the polypeptide, the first polypeptide monomer, and/or the second polypeptide monomer comprises an H524 substitution, optionally wherein the H524 substitution is an H524L substitution or an H524F substitution; (e) the one or more additional amino acid substitutions of the polypeptide, the first polypeptide monomer, and/or the second polypeptide monomer comprises an M421L substitution; (f) the one or more additional amino acid substitutions of the polypeptide, the first polypeptide monomer, and/or the second polypeptide monomer comprises an S463P substitution; (g) the one or more additional amino acid substitutions of the polypeptide, the first polypeptide monomer, and/or the second polypeptide monomer comprises an M421L substitution and an S463P substitution; (h) the one or more additional amino acid substitutions of the polypeptide, the first polypeptide monomer, and/or the second polypeptide monomer comprises an L384M substitution; (i) the one or more additional amino acid substitutions of the polypeptide, the first polypeptide monomer, and/or the second polypeptide monomer comprises a L354I substitution; (j) the one or more additional amino acid substitutions of the polypeptide, the first polypeptide monomer, and/or the second polypeptide monomer comprises a Q414E substitution; (k) the one or more additional amino acid substitutions of the polypeptide, the first polypeptide monomer, and/or the second polypeptide monomer comprises a L354I substitution and a Q414E substitution; (1) the one or more additional amino acid substitutions of the polypeptide, the first polypeptide monomer, and/or the second polypeptide monomer comprises an L391V substitution, an N413D mutation, and an H524 substitution, optionally wherein the H524 substitution is an H524L substitution or an H524F substitution; (m) the one or more additional amino acid substitutions of the polypeptide, the first polypeptide monomer, and/or the second polypeptide monomer comprises an L391V substitution, an N413D mutation, an H524 substitution, and an M421L substitution, optionally wherein the H524 substitution is an H524L substitution or an H524F substitution; (n) the one or more additional amino acid substitutions of the polypeptide, the first polypeptide monomer, and/or the second polypeptide monomer comprises an L391V substitution, an N413D mutation, an H524 substitution, and an S463P substitution, optionally wherein the H524 substitution is an H524L substitution or an H524F substitution; (o) wherein the one or more additional amino acid substitutions of the polypeptide, the first polypeptide monomer, and/or the second polypeptide monomer comprises an L391V substitution, an N413D mutation, an H524 substitution, and an Q414E substitution, optionally wherein the H524 substitution is an H524L substitution or an H524F substitution; (p) the one or more additional amino acid substitutions of the polypeptide, the first polypeptide monomer, and/or the second polypeptide monomer comprises an L391V substitution, an N413D mutation, an H524 substitution, and an L354I substitution, optionally wherein the H524 substitution is an H524L substitution or an H524F substitution.

In some embodiments, the one or more additional amino acid substitutions of the polypeptide, the first polypeptide monomer, and/or the second polypeptide monomer are at one or more positions of SEQ ID NO: 1 selected from: 343, 344, 345, 346, 347, 348, 349, 350, 351, 352, 354, 380, 384, 386, 387, 388, 389, 391, 392, 404, 407, 409, 413, 414, 417, 418, 420, 421, 422, 424, 428, 463, 517, 521, 522, 524, 525, 526, 527, 528, 533, 534, 536, 537, 538, 539, 540, and 547, optionally wherein: (i) the one or more positions comprise position 343 of SEQ ID NO: 1, optionally wherein the amino acid substitution at position 343 of SEQ ID NO: 1 is selected from the group consisting of: M343F, M343I, M343L, and M343V; (ii) the one or more positions comprise position 344 of SEQ ID NO: 1, optionally wherein the amino acid substitution at position 344 of SEQ ID NO: 1 is G344M; (iii) the one or more positions comprise position 345 of SEQ ID NO: 1, optionally wherein the amino acid substitution at position 345 of SEQ ID NO: 1 is L345S; (iv) the one or more positions comprise position 346 of SEQ ID NO: 1, optionally wherein the amino acid substitution at position 346 of SEQ ID NO: 1 is selected from the group consisting of: L346I, L346M, L346F, and L346V; (v) the one or more positions comprise position 347 of SEQ ID NO: 1, optionally wherein the amino acid substitution at position 347 of SEQ ID NO: 1 is selected from the group consisting of: T347D, T347E, T347F, T347I, T347K, T347L, T347M, T347N, T347Q, T347R, T347S, and T347V; (vi) the one or more positions comprise position 348 of SEQ ID NO: 1, optionally wherein the amino acid substitution at position 348 of SEQ ID NO: 1 is N348K; (vii) the one or more positions comprise position 349 of SEQ ID NO: 1, optionally wherein the amino acid substitution at position 349 of SEQ ID NO: 1 is selected from the group consisting of: L349I, L349M, L349F, and L349V; (viii) the one or more positions comprise position 350 of SEQ ID NO: 1, optionally wherein the amino acid substitution at position 350 of SEQ ID NO: 1 is selected from the group consisting of: A350F, A350I, A350L, A350M and A350V; (ix) the one or more positions comprise position 351 of SEQ ID NO: 1, optionally wherein the amino acid substitution at position 351 of SEQ ID NO: 1 is selected from the group consisting of: D351E, D351F, D351I, D351L, D351M, D351N, D351Q, and D351V; (x) the one or more positions comprise position 352 of SEQ ID NO: 1, optionally wherein the amino acid substitution at position 352 of SEQ ID NO: 1 is R352K; (xi) the one or more positions comprise position 354 of SEQ ID NO: 1, optionally wherein the amino acid substitution at position 354 of SEQ ID NO: 1 is selected from the group consisting of: L354I, L354M, L354F, and L354V; (xii) the one or more positions comprise position 380 of SEQ ID NO: 1, optionally wherein the amino acid substitution at position 380 of SEQ ID NO: 1 is E380Q; (xiii) the one or more positions comprise position 384 of SEQ ID NO: 1, optionally wherein the amino acid substitution at position 384 of SEQ ID NO: 1 is selected from the group consisting of: L384I, L384M, L384F, and L384V; (xiv) the one or more positions comprise position 386 of SEQ ID NO: 1, optionally wherein the amino acid substitution at position 386 of SEQ ID NO: 1 is 1386V; (xv) the one or more positions comprise position 387 of SEQ ID NO: 1, optionally wherein the amino acid substitution at position 387 of SEQ ID NO: 1 is selected from the group consisting of: L387I, L387M, L387F, and L387V; (xvi) the one or more positions comprise position 388 of SEQ ID NO: 1, optionally wherein the amino acid substitution at position 388 of SEQ ID NO: 1 is selected from the group consisting of: M388I, M388L, and M388F; (xvii) the one or more positions comprise position 389 of SEQ ID NO: 1, optionally wherein the amino acid substitution at position 389 of SEQ ID NO: 1 is 1389M; (xviii) the one or more positions comprise position 391 of SEQ ID NO: 1, optionally wherein the amino acid substitution at position 391 of SEQ ID NO: 1 is selected from the group consisting of: L391I, L391M, L391F, and L391V, optionally wherein the amino acid substitution at position 391 of SEQ ID NO: 1 is L391V; (xix) the one or more positions comprise position 392 of SEQ ID NO: 1, optionally wherein the amino acid substitution at position 392 of SEQ ID NO: 1 is V392M; (xx) the one or more positions comprise position 404 of SEQ ID NO: 1, optionally wherein the amino acid substitution at position 404 of SEQ ID NO: 1 is selected from the group consisting of: F404I, F404L, F404M, and F404V; (xxi) the one or more positions comprise position 407 of SEQ ID NO: 1, optionally wherein the amino acid substitution at position 407 of SEQ ID NO: 1 is N407D; (xxii) the one or more positions comprise position 409 of SEQ ID NO: 1, optionally wherein the amino acid substitution at position 409 of SEQ ID NO: 1 is L409V; (xxiii) the one or more positions comprise position 413 of SEQ ID NO: 1, optionally wherein the amino acid substitution at position 413 of SEQ ID NO: 1 is N413D; (xxiv) the one or more positions comprise position 414 of SEQ ID NO: 1, optionally wherein the amino acid substitution at position 414 of SEQ ID NO: 1 is Q414E; (xxv) the one or more positions comprise position 417 of SEQ ID NO: 1, optionally wherein the amino acid substitution at position 417 of SEQ ID NO: 1 is C417S; (xxvi) the one or more positions comprise position 418 of SEQ ID NO: 1, optionally the amino acid substitution at position 418 of SEQ ID NO: 1 is selected from the group consisting of: V418I, V418L, V418M, and V418F; (xxvii) the one or more positions comprise position 420 of SEQ ID NO: 1, optionally wherein the amino acid substitution at position 420 of SEQ ID NO: 1 is selected from the group consisting of: G420I, G420M, G420F, and G420V; (xxviii) the one or more positions comprise position 421 of SEQ ID NO: 1, optionally wherein the amino acid substitution at position 421 of SEQ ID NO: 1 is selected from the group consisting of: M421I, M421L, M421F, and M421V; (xxix) the one or more positions comprise position 422 of SEQ ID NO: 1, optionally wherein the amino acid substitution at position 422 of SEQ ID NO: 1 is V422I; (xxx) the one or more positions comprise position 424 of SEQ ID NO: 1, optionally wherein the amino acid substitution at position 424 of SEQ ID NO: 1 is selected from the group consisting of: 1424L, 1424M, 1424F, and I424V; (xxxi) the one or more positions comprise position 428 of SEQ ID NO: 1, optionally wherein the amino acid substitution at position 428 of SEQ ID NO: 1 is selected from the group consisting of: L428I, L428M, L428F, and L428V; (xxxii) the one or more positions comprise position 463 of SEQ ID NO: 1, optionally wherein the amino acid substitution at position 463 of SEQ ID NO: 1 is S463P; (xxxiii) the one or more positions comprise position 517 of SEQ ID NO: 1, optionally wherein the amino acid substitution at position 517 of SEQ ID NO: 1 is M517A; (xxxiv) the one or more positions comprise position 521 of SEQ ID NO: 1, optionally wherein the amino acid substitution at position 521 of SEQ ID NO: 1 is selected from the group consisting of: G521A, G521F, G521I, G521L, G521M, and G521V; (xxxv) the one or more positions comprise position 522 of SEQ ID NO: 1, optionally wherein the amino acid substitution at position 522 of SEQ ID NO: 1 is selected from the group consisting of: M522I, M522L, and M522V; (xxxvi) the one or more positions comprise position 524 of SEQ ID NO: 1, optionally wherein the amino acid substitution at position 524 of SEQ ID NO: 1 is selected from the group consisting of: H524A, H524I, H524L, H524F, and H524V; (xxxvii) the one or more positions comprise position 525 of SEQ ID NO: 1, optionally wherein the amino acid substitution at position 525 of SEQ ID NO: 1 is selected from the group consisting of: L525F, L525I, L525M, L525N, L525Q, L525S, L525T, and L525V; (xxxviii) the one or more positions comprise position 526 of SEQ ID NO: 1, optionally wherein the amino acid substitution at position 526 of NO: 1 is Y526L; (xxxix) the one or more positions comprise position 527 of SEQ ID NO: 1, optionally wherein the amino acid substitution at position 527 of SEQ ID NO: 1 is S527N; (xl) the one or more positions comprise position 528 of SEQ ID NO: 1, optionally wherein the amino acid substitution at position 528 of SEQ ID NO: 1 is selected from the group consisting of: M528F, M528I, and M528V; (xli) the one or more positions comprise position 533 of SEQ ID NO: 1, optionally wherein the amino acid substitution at position 533 of SEQ ID NO: 1 is selected from the group consisting of: V533F and V533W; (xlii) the one or more positions comprise position 534 of SEQ ID NO: 1, optionally wherein the amino acid substitution at position 534 of SEQ ID NO: 1 is selected from the group consisting of: V534Q and V534R; (xliii) the one or more positions comprise position 536 of SEQ ID NO: 1, optionally wherein the amino acid substitution at position 536 of SEQ ID NO: 1 is selected from the group consisting of: L536F, and L536M, L536R, and L536Y; (xliv) the one or more positions comprise position 537 of SEQ ID NO: 1, optionally wherein the amino acid substitution at position 537 of SEQ ID NO: 1 is selected from the group consisting of: Y537E and Y537S; (xlv) the one or more positions comprise position 538 of SEQ ID NO: 1, optionally wherein the amino acid substitution at position 538 of SEQ ID NO: 1 is selected from the group consisting of: D538G and D538K; (xlvi) the one or more positions comprise position 539 of SEQ ID NO: 1, optionally wherein the amino acid substitution at position 539 of SEQ ID NO: 1 is selected from the group consisting of: L539A and L539R; (xlvii) the one or more positions comprise position 540 of SEQ ID NO: 1, optionally wherein the amino acid substitution at position 540 of SEQ ID NO: 1 is selected from the group consisting of: L540A and L540F; and/or (xlviii) the one or more positions comprise position 547 of SEQ ID NO: 1, optionally wherein the amino acid substitution at position 547 of SEQ ID NO: 1 is H547A.

In some embodiments, (a) the one or more additional amino acid substitutions of the polypeptide, the first polypeptide monomer, and/or the second polypeptide monomer are two amino acid substitutions, optionally wherein each of the two amino acid substitutions are at a position of SEQ ID NO: 1 selected from: 343, 345, 347, 348, 351, 354, 384, 387, 388, 389, 391, 392, 404, 418, 421, 521, 524, and 525, optionally wherein: (i) the two amino acid substitutions are at positions 345 and 348 of SEQ ID NO: 1, optionally wherein the amino acid substitution at position 345 of SEQ ID NO: 1 is L345S and the amino acid substitution at position 348 of SEQ ID NO: 1 is N348K; (ii) the two amino acid substitutions are at positions 384 and 389 of SEQ ID NO: 1, optionally wherein the amino acid substitution at position 384 of SEQ ID NO: 1 is L384M and the amino acid substitution at position 389 of SEQ ID NO: 1 is 1389M; (iii) the two amino acid substitutions are at positions 421 and 392 of SEQ ID NO: 1, optionally wherein the amino acid substitution at position 421 of SEQ ID NO: 1 is M421I and the amino acid substitution at position 392 of SEQ ID NO: 1 is V392M; (iv) the two amino acid substitutions are at positions 354 and 391 of SEQ ID NO: 1, optionally wherein the amino acid substitution at position 354 of SEQ ID NO: 1 is L354I and the amino acid substitution at position 391 of SEQ ID NO: 1 is L391F; (v) the two amino acid substitutions are at positions 354 and 384 of SEQ ID NO: 1, optionally wherein the amino acid substitution at position 354 of SEQ ID NO: 1 is L354I and the amino acid substitution at position 384 of SEQ ID NO: 1 is L384M; (vi) the two amino acid substitutions are at positions 354 and 387 of SEQ ID NO: 1, optionally wherein the amino acid substitution at position 354 of SEQ ID NO: 1 is L354I and the amino acid substitution at position 387 of SEQ ID NO: 1 is L387M; (vii) the two amino acid substitutions are at positions 387 and 391, optionally wherein the amino acid substitution at position 387 of SEQ ID NO: 1 is L387M and the amino acid substitution at position 391 of SEQ ID NO: 1 is L391F; (viii) the two amino acid substitutions are at positions 384 and 387 of SEQ ID NO: 1, optionally wherein the amino acid substitution at position 384 of SEQ ID NO: 1 is L384M and the amino acid substitution at position 387 of SEQ ID NO: 1 is L387M; or (ix) the two amino acid substitutions are at positions 384 and 391 of SEQ ID NO: 1, optionally wherein the amino acid substitution at position 384 of SEQ ID NO: 1 is L384M and the amino acid substitution at position 391 of SEQ ID NO: 1 is L391F; (b) the one or more additional amino acid substitutions of the polypeptide, the first polypeptide monomer, and/or the second polypeptide monomer are three amino acid substitutions, optionally wherein each of the three amino acid substitutions are at a position of SEQ ID NO: 1 selected from: 343, 347, 351, 354, 388, 391, 404, 414, 418, 463, 521, 524, and 525, optionally wherein: (i) the three amino acid substitutions are at positions 354, 384, and 391 of SEQ ID NO: 1, optionally wherein the amino acid substitution at position 354 of SEQ ID NO: 1 is L354I, the amino acid substitution at position 384 of SEQ ID NO: 1 is L384M, and the amino acid substitution at position 391 of SEQ ID NO: 1 is L391F; (ii) the three amino acid substitutions are at positions 414, 463, and 524 of SEQ ID NO: 1, optionally wherein the amino acid substitution at position 414 of SEQ ID NO: 1 is Q414E, the amino acid substitution at position 463 of SEQ ID NO: 1 is S463P, and the amino acid substitution at position 524 of SEQ ID NO: 1 is H524L; (c) the one or more additional amino acid substitutions of the polypeptide, the first polypeptide monomer, and/or the second polypeptide monomer are four amino acid substitutions, optionally wherein each of the four amino acid substitutions are at a position of SEQ ID NO: 1 selected from: 343, 347, 351, 354, 384, 388, 391, 404, 413, 418, 463, 521, 524, and 525, optionally wherein: (i) the four amino acid substitutions are at positions 354, 384, 391, and 418 of SEQ ID NO: 1, optionally wherein the amino acid substitution at position 354 of SEQ ID NO: 1 is L354I, the amino acid substitution at position 384 of SEQ ID NO: 1 is L384M, the amino acid substitution at position 391 of SEQ ID NO: 1 is L391F, and the amino acid substitution at position 418 of SEQ ID NO: 1 is V418I; (ii) the four amino acid substitutions are at positions 343, 388, 521, and 404 of SEQ ID NO: 1, optionally wherein the amino acid substitution at position 343 of SEQ ID NO: 1 is M343I, the amino acid substitution at position 388 of SEQ ID NO: 1 is M388I, the amino acid substitution at position 521 of SEQ ID NO: 1 is G521I, and the amino acid substitution at position 404 of SEQ ID NO: 1 is F404L; (iii) the four amino acid substitutions are at positions 524, 347, 351, and 525 of SEQ ID NO: 1, optionally wherein the amino acid substitution at position 524 of SEQ ID NO: 1 is H524V, the amino acid substitution at position 347 of SEQ ID NO: 1 is T347R, the amino acid substitution at position 351 of SEQ ID NO: 1 is D351Q, and the amino acid substitution at position 525 of SEQ ID NO: 1 is L525N; (iv) the four amino acid substitutions are at positions 354, 384, 391, and 463 of SEQ ID NO: 1, optionally wherein the amino acid substitution at position 354 of SEQ ID NO: 1 is L354I, the amino acid substitution at position 384 of SEQ ID NO: 1 is L384M, the amino acid substitution at position 391 of SEQ ID NO: 1 is L391V, and the amino acid substitution at position 463 of SEQ ID NO: 1 is S463P; or (v) the four amino acid substitutions are at positions 384, 391, 413, and 524 of SEQ ID NO: 1, optionally wherein the amino acid substitution at position 384 of SEQ ID NO: 1 is L384M, the amino acid substitution at position 391 of SEQ ID NO: 1 is L391V, the amino acid substitution at position 413 of SEQ ID NO: 1 is N413D, and the amino acid substitution at position 524 of SEQ ID NO: 1 is H524F; (d) the one or more additional amino acid substitutions of the polypeptide, the first polypeptide monomer, and/or the second polypeptide monomer are five amino acid substitutions, optionally wherein each of the five amino acid substitutions are at a position of SEQ ID NO: 1 selected from: 354, 384, 391, 409, 413, 414, 421, 463, and 524, optionally wherein (i) the five amino acid substitutions are at positions 384, 409, 413, 463, and 524 of SEQ ID NO: 1, optionally wherein the amino acid substitution at position 384 of SEQ ID NO: 1 is L384M, the amino acid substitution at position 409 of SEQ ID NO: 1 is L409V, the amino acid substitution at position 413 of SEQ ID NO: 1 is N413D, the amino acid substitution at position 463 of SEQ ID NO: 1 is S463P, and the amino acid substitution at position 524 of SEQ ID NO: 1 is H524L; (ii) the five amino acid substitutions are at positions 391, 413, 414, 463, and 524 of SEQ ID NO: 1, optionally wherein the amino acid substitution at position 391 of SEQ ID NO: 1 is L391V, the amino acid substitution at position 413 of SEQ ID NO: 1 is N413D, the amino acid substitution at position 414 of SEQ ID NO: 1 is Q414E, the amino acid substitution at position 463 of SEQ ID NO: 1 is S463P, and the amino acid substitution at position 524 of SEQ ID NO: 1 is H524F; (iii) the five amino acid substitutions are at positions 391, 414, 421, 463, and 524 of SEQ ID NO: 1, optionally wherein the amino acid substitution at position 391 of SEQ ID NO: 1 is L391V, the amino acid substitution at position 414 of SEQ ID NO: 1 is Q414E, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524F; (iv) the five amino acid substitutions are at positions 354, 409, 413, 421, and 524 of SEQ ID NO: 1, optionally wherein the amino acid substitution at position 354 of SEQ ID NO: 1 is L354I, the amino acid substitution at position 409 of SEQ ID NO: 1 is L409V, the amino acid substitution at position 413 of SEQ ID NO: 1 is N413D, the amino acid substitution at position 421 of SEQ ID NO: 1 is M421L, and the amino acid substitution at position 524 of SEQ ID NO: 1 is H524L; or (v) the five amino acid substitutions are at positions 354, 409, 421, 463, and 524 of SEQ ID NO: 1, optionally wherein the amino acid substitution at position 354 of SEQ ID NO: 1 is L354I, the amino acid substitution at position 409 of SEQ ID NO: 1 is L409V, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524L; (c) the one or more additional amino acid substitutions of the polypeptide, the first polypeptide monomer, and/or the second polypeptide monomer are six amino acid substitutions, optionally wherein each of the six amino acid substitutions are at a position of SEQ ID NO: 1 selected from: 354, 384, 391, 409, 413, 414, 421, 463, and 524, optionally wherein: (i) the six amino acid substitutions are at positions 384, 391, 413, 421, 463, and 524 of SEQ ID NO: 1, optionally wherein the amino acid substitution at position 384 of SEQ ID NO: 1 is L384M, the amino acid substitution at position 391 of SEQ ID NO: 1 is L391V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524L; (ii) the six amino acid substitutions are at positions 409, 413, 414, 421, 463, and 524 of SEQ ID NO: 1; optionally wherein the amino acid substitution at position 409 of SEQ ID NO: 1 is L409V, the amino acid substitution at position 413 of SEQ ID NO: 1 is N413D, the amino acid substitution at position 414 of SEQ ID NO: 1 is Q414E, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524L; or (iii) the six amino acid substitutions are at positions 354, 391, 409, 413, 414, and 524 of SEQ ID NO: 1, optionally wherein the amino acid substitution at position 354 of SEQ ID NO: 1 is L354I, the amino acid substitution at position 391 of SEQ ID NO: 1 is L391V, the amino acid substitution at position 409 of SEQ ID NO: 1 is L409V, the amino acid substitution at position 413 of SEQ ID NO: 1 is N413D, the amino acid substitution at position 414 of SEQ ID NO: 1 is Q414E, and the amino acid substitution at position 524 of SEQ ID NO: 1 is H524L; (f) the one or more additional amino acid substitutions of the polypeptide, the first polypeptide monomer, and/or the second polypeptide monomer are seven amino acid substitutions, optionally wherein each of the seven amino acid substitutions are at a position of SEQ ID NO: 1 selected from: 354, 384, 391, 409, 413, 414, 421, 463, 517, and 524, optionally wherein: (i) the seven amino acid substitutions are at positions 354, 384, 409, 413, 421, 463, and 524 of SEQ ID NO: 1, optionally wherein the amino acid substitution at position 354 of SEQ ID NO: 1 is L354I, the amino acid substitution at position 384 of SEQ ID NO: 1 is L384M, the amino acid substitution at position 409 of SEQ ID NO: 1 is L409V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524F; (ii) the seven amino acid substitutions are at positions 354, 391, 413, 421, 463, 517, and 524 of SEQ ID NO: 1, optionally wherein the amino acid substitution at position 354 of SEQ ID NO: 1 is L354I, the amino acid substitution at position 391 of SEQ ID NO: 1 is L391V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, the amino acid substitution at position 517 of SEQ ID NO: 1 is M517A, and the amino acid substitution at position 524 of SEQ ID NO: 1 is H524L; or (iii) the seven amino acid substitutions are at positions 354, 391, 413, 414, 421, 517, and 524 of SEQ ID NO: 1; optionally wherein the amino acid substitution at position 354 of SEQ ID NO: 1 is L354I, the amino acid substitution at position 391 of SEQ ID NO: 1 is L391V, the amino acid substitution at position 413 of SEQ ID NO: 1 is N413D, the amino acid substitution at position 414 of SEQ ID NO: 1 is Q414E, the amino acid substitution at position 421 of SEQ ID NO: 1 is M421L, the amino acid substitution at position 517 of SEQ ID NO: 1 is M517A, and the amino acid substitution at position 524 of SEQ ID NO: 1 is H524F; or (g) the one or more additional amino acid substitutions of the polypeptide, the first polypeptide monomer, and/or the second polypeptide monomer are eight amino acid substitutions, optionally wherein the eight amino acid substitutions are at positions 384, 391, 409, 413, 421, 463, 517, and 524 of SEQ ID NO: 1, optionally wherein (i) the amino acid substitution at position 384 of SEQ ID NO: 1 is L384M, the amino acid substitution at position 391 of SEQ ID NO: 1 is L391V, the amino acid substitution at position 409 of SEQ ID NO: 1 is L409V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, the amino acid substitution at position 517 of SEQ ID NO: 1 is M517A, and the amino acid substitution at position 524 of SEQ ID NO: 1 is H524F.

Also provided herein, in various embodiments, is an inducible cell death system comprising a first polypeptide and a second polypeptide monomer, wherein the first and the second polypeptide monomers each comprise a ligand binding domain and a cell death inducing domain, wherein the first and the second polypeptide monomers are configured to oligomerize upon contact with a ligand of the ligand binding domain, thereby generating a cell-death inducing signal in a cell in which the first and the second polypeptide monomers are expressed, and wherein the ligand binding domain comprises a modified estrogen receptor ligand binding domain (ER-LBD) comprising an amino acid sequence corresponding to a hormone binding domain of a reference human estrogen receptor sequence (SEQ ID NO: 1), wherein the modified ER-LBD comprises: (a) a G400V amino acid substitution, an M543A amino acid substitution, an L544A amino acid substitution, and optionally a V595A amino acid substitution, with reference to SEQ ID NO: 1; and (b) additional amino acid substitutions, wherein the additional amino acid substitutions comprise, with reference to SEQ ID NO: 1: (i) an L384M substitution, an L391V substitution, a N413D substitution, an M421L substitution, a S463P substitution, and a H524L substitution, (ii) an L391V substitution, a N413D substitution, a Q414E substitution, a S463P substitution, and a H524F substitution, (iii) an L354I substitution, a L391V substitution, a N413D substitution, a Q414E substitution, a M421L substitution, a M517A substitution, and a H524F substitution, or (iv) an L354I substitution, a L391V substitution, a L409V substitution, a N413D substitution, a Q414E substitution, and a H524L substitution.

In some embodiments, the one or more additional amino acid substitutions of the polypeptide, the first polypeptide monomer, and/or the second polypeptide monomer comprises an N413D mutation, an H524 substitution, and an S463P substitution, optionally wherein: (a) the one or more additional amino acid substitutions of the polypeptide, the first polypeptide monomer, and/or the second polypeptide monomer comprise an L391V substitution, an L409V substitution, an Q414E substitution, an N413D substitution, an S463P substitution, an M517A substitution, and an H524L substitution, optionally wherein: the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 90 or 103; (b) the one or more additional amino acid substitutions of the polypeptide, the first polypeptide monomer, and/or the second polypeptide monomer comprise an L409V substitution, an N413D substitution, an S463P substitution, an M421L substitution, an L384M substitution, and an H524L substitution, optionally wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 91 or 104; (c) the one or more additional amino acid substitutions of the polypeptide, the first polypeptide monomer, and/or the second polypeptide monomer comprise an L391V substitution, an L409V substitution, an N413D substitution, an S463P substitution, an M517A substitution, an M421L substitution, an L354I substitution, and an H524L substitution, optionally wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 92 or 105; (d) the one or more additional amino acid substitutions of the polypeptide, the first polypeptide monomer, and/or the second polypeptide monomer comprise an L391V substitution, an Q414E substitution, an N413D substitution, an S463P substitution, an M421L substitution, an L354I substitution, an L384M substitution, and an H524L substitution, optionally wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 93 or 106; (e) the one or more additional amino acid substitutions of the polypeptide, the first polypeptide monomer, and/or the second polypeptide monomer comprise an L391V substitution, an L409V substitution, an N413D substitution, an S463P substitution, an M517A substitution, an M421L substitution, and an H524L substitution, optionally wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 94 or 107; (f) the one or more additional amino acid substitutions of the polypeptide, the first polypeptide monomer, and/or the second polypeptide monomer comprise an L391V substitution, an L409V substitution, an Q414E substitution, an N413D substitution, an S463P substitution, an L354I substitution, and an H524L substitution, optionally wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 95 or 108; (g) the one or more additional amino acid substitutions of the polypeptide, the first polypeptide monomer, and/or the second polypeptide monomer comprise an L391V substitution, an L409V substitution, an N413D substitution, an S463P substitution, an M421L substitution, an L354I substitution, an L384M substitution, and an H524L substitution, optionally wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 96 or 109; (h) the one or more additional amino acid substitutions of the polypeptide, the first polypeptide monomer, and/or the second polypeptide monomer comprise an L391V substitution, an Q414E substitution, an N413D substitution, an S463P substitution, an M517A substitution, an M421L substitution, an L354I substitution, and an H524L substitution, optionally wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 97 or 110; (i) the one or more additional amino acid substitutions of the polypeptide, the first polypeptide monomer, and/or the second polypeptide monomer comprise an L391V substitution, an N413D substitution, an S463P substitution, an M517A substitution, an L384M substitution, and an H524L substitution, optionally wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 98 or 111; (j) the one or more additional amino acid substitutions of the polypeptide, the first polypeptide monomer, and/or the second polypeptide monomer comprise an L391V substitution, an L409V substitution, an N413D substitution, an S463P substitution, an M517A substitution, and an H524L substitution, optionally wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 99 or 112; (k) the one or more additional amino acid substitutions of the polypeptide, the first polypeptide monomer, and/or the second polypeptide monomer comprise an N413D substitution, an S463P substitution, an L354I substitution, an L384M substitution, and an H524L substitution, optionally wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 100 or 113; or (1) the one or more additional amino acid substitutions of the polypeptide, the first polypeptide monomer, and/or the second polypeptide monomer comprise an N413D substitution, an S463P substitution, an M421L substitution, an L354I substitution, and an H524L substitution, optionally wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 101 or 114, optionally wherein the ligand is a non-endogenous ligand, optionally wherein the non-endogenous ligand is selected from: 4-hydroxytamoxifen, N-desmethyltamoxifen, tamoxifen-N-oxide, and endoxifen, optionally wherein the non-endogenous ligand comprises a tamoxifen metabolite, optionally wherein the non-endogenous ligand is endoxifen.

In some embodiments, the first polypeptide monomer and the second polypeptide monomer are capable of oligomerization and/or generating the cell-death inducing signal at a concentration of: (a) 0.25 nM Endoxifen or less and/or at a concentration of 0.04 nM 4-OHT or less; (b) 2.5 nM Endoxifen or less and/or at a concentration of 0.4 nM 4-OHT or less; (c) at least 0.001 pM of 4-OHT; or (d) at least 0.01 pM of 4-OHT.

In some embodiments, the cell death-inducing domain is: (a) derived from a protein selected from: caspase 3, caspase 6, caspase 7, caspase 8, caspase 9, Diphtheria toxin fragment A (DTA), Bax, Bak, Bok, Bad, Bcl-Xs, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B1, and Purine nucleoside phosphorylase, optionally wherein the cell death-inducing domain comprises the Caspase 9 derived amino acid sequence of SEQ ID NO: 48 or 125, optionally wherein the caspase domain or functional fragment thereof does not comprise a Caspase Activation and Recruitment Domain (CARD) domain sequence; or (b) a transcription factor comprising a nucleic acid-binding domain and a transcriptional effector domain, wherein the transcription factor is configured to generate a cell-death inducing signal by inducing expression of: a caspase domain or functional fragment thereof, optionally wherein the caspase is selected from caspase 3, caspase 6, caspase 7, caspase 8, caspase 9, or functional fragments thereof, respectively, Diphtheria toxin fragment A (DTA), Bax, Bak, Bok, Bad, Bcl-Xs, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B1, or Purine nucleoside phosphorylase.

Also provided herein, in various embodiments, is an isolated polynucleotide comprising a nucleotide sequence encoding the polypeptide, the first polypeptide monomer, and/or the second polypeptide monomer provided herein.

Also provided herein, in various embodiments, is a heterologous construct comprising a promoter operatively linked to the polynucleotide provided herein.

Also provided herein, in various embodiments, is a plasmid or a vector comprising the heterologous construct provided herein.

Also provided herein, in various embodiments, is a cell comprising the heterologous construct, the plasmid, or the vector provided herein.

Also provided herein, in various embodiments, is a molecular switch for generating a cell-death inducing signal in a cell, comprising: (a) the inducible cell death system, the isolated polynucleotide, the heterologous construct, the plasmid, the vector, or the cell provided herein, wherein the inducible cell death system is capable of generating a cell-death inducing signal in the cell; and (b) a non-endogenous ligand, wherein binding of the non-endogenous ligand to the modified ER-LBD generates the cell-death inducing signal in the cell, optionally wherein the non-endogenous ligand is selected from: 4-hydroxytamoxifen, N-desmethyltamoxifen, tamoxifen-N-oxide, and endoxifen, optionally wherein the non-endogenous ligand comprises a tamoxifen metabolite, optionally wherein the non-endogenous ligand is endoxifen, optionally wherein: (i) the first polypeptide monomer and the second polypeptide monomer are capable of oligomerization and/or generating the cell-death inducing signal at a concentration of 0.25 nM Endoxifen or less and/or at a concentration of 0.04 nM 4-OHT or less; (ii) the first polypeptide monomer and the second polypeptide monomer are capable of oligomerization and/or generating the cell-death inducing signal at a concentration of 2.5 nM Endoxifen or less and/or at a concentration of 0.4 nM 4-OHT or less; (iii) the first polypeptide monomer and the second polypeptide monomer are capable of oligomerization and/or generating the cell-death inducing signal at a concentration of at least 0.001 pM of 4-OHT; or (iv) the first polypeptide monomer and the second polypeptide monomer are capable of oligomerization and/or generating the cell-death inducing signal at a concentration of at least 0.01 pM of 4-OHT.

Also provided herein, in various embodiments, is a method of inducing oligomerization of a chimeric protein comprising: transforming a cell with (i) a heterologous construct encoding any one of the inducible cell death systems of any one of claims 1 to 8, the isolated polynucleotide of claim 9, the heterologous construct of claim 10, or the plasmid or vector of claim 11, and (ii) contacting the transformed cell with a non-endogenous ligand of the modified estrogen receptor ligand binding domain (ER-LBD), optionally wherein (a) the method further comprising culturing the transformed cell under conditions suitable for expression of the of the inducible cell death system prior to inducing oligomerization and/or inducing cell death; (b) the transformed cell is in a human or animal, and wherein contacting the transformed cell with the non-endogenous ligand comprises administering a pharmacological dose of the ligand to the human or animal; and/or (c) the non-endogenous ligand is selected from: 4-hydroxytamoxifen, N-desmethyltamoxifen, tamoxifen-N-oxide, and endoxifen, optionally wherein the non-endogenous ligand comprises a tamoxifen metabolite, optionally wherein the non-endogenous ligand is endoxifen, optionally wherein the non-endogenous ligand is administered at a concentration at which the non-endogenous ligand is substantially inactive on a wild-type estrogen receptor alpha of SEQ ID NO: 1.

Also provided herein, in various embodiments, is a modified estrogen receptor ligand binding domain (ER-LBD) corresponding to a hormone binding domain of a reference human estrogen receptor sequence (SEQ ID NO: 1), wherein: (i) the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an N413D substitution, an S463P substitution, an L354I substitution, an L384M substitution, and an H524L substitution, with reference to SEQ ID NO: 1; (ii) the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an N413D substitution, an S463P substitution, an M421L substitution, an L354I substitution, and an H524L substitution, with reference to SEQ ID NO: 1; (iii) the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L409V substitution, an N413D substitution, an S463P substitution, an M421L substitution, an L384M substitution, and an H524L substitution, with reference to SEQ ID NO: 1; (iv) the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an L409V substitution, an Q414E substitution, an N413D substitution, an S463P substitution, an M517A substitution, and an H524L substitution, with reference to SEQ ID NO: 1; (v) the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an L409V substitution, an N413D substitution, an S463P substitution, an M517A substitution, an M421L substitution, an L354I substitution, and an H524L substitution, with reference to SEQ ID NO: 1; (vi) the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an Q414E substitution, an N413D substitution, an S463P substitution, an M421L substitution, an L354I substitution, an L384M substitution, and an H524L substitution, with reference to SEQ ID NO: 1; (vii) the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an L409V substitution, an N413D substitution, an S463P substitution, an M517A substitution, an M421L substitution, and an H524L substitution, with reference to SEQ ID NO: 1; (viii) the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an L409V substitution, an Q414E substitution, an N413D substitution, an S463P substitution, an L354I substitution, and an H524L substitution, with reference to SEQ ID NO: 1; (ix) the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an L409V substitution, an N413D substitution, an S463P substitution, an M421L substitution, an L354I substitution, an L384M substitution, and an H524L substitution, with reference to SEQ ID NO: 1; (x) the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an Q414E substitution, an N413D substitution, an S463P substitution, an M517A substitution, an M421L substitution, an L354I substitution, and an H524L substitution, with reference to SEQ ID NO: 1; (xi) the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an N413D substitution, an S463P substitution, an M517A substitution, an L384M substitution, and an H524L substitution, with reference to SEQ ID NO: 1; (xii) the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an L409V substitution, an N413D substitution, an S463P substitution, an M517A substitution, and an H524L substitution, with reference to SEQ ID NO: 1; or (xiii) the modified ER-LBD comprises an amino acid sequence having at least 80%, 85%, 90%, 95%, 97.5%, 98%, 99%, or 100% identical to any one of SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, and SEQ ID NO: 114, optionally wherein the modified ER-LBD has greater sensitivity and/or selectivity to a non-endogenous ligand as compared to an ER-LBD comprising the amino acid sequence of SEQ ID NO: 2, or as compared to an endogenous ligand as a result of the one or more additional amino acid substitutions, optionally wherein the non-endogenous ligand is selected from the group consisting of: 4-hydroxytamoxifen, N-desmethyltamoxifen, tamoxifen-N-oxide, tamoxifen, and endoxifen, optionally wherein the endogenous ligand is estradiol, optionally wherein the modified ER-LBD further comprises a V595A amino acid substitution.

Also provided herein, in various embodiments, is a chimeric protein comprising a polypeptide of interest fused to the modified ER-LBD of claim 15, optionally wherein the polypeptide of interest comprises a nucleic acid binding domain, optionally wherein the nucleic acid binding domain comprises a zinc finger domain, optionally wherein the zinc finger domain comprises the sequence as set forth in SEQ ID NO: 57 or SEQ ID NO: 84, optionally wherein the chimeric protein comprises a chimeric transcription factor, and wherein the polypeptide of interest comprises a nucleic acid binding domain and a transcriptional modulator domain, optionally wherein the transcriptional modular domain is a transcriptional activator, optionally wherein the transcriptional activator is selected from the group consisting of: a Herpes Simplex Virus Protein 16 (VP16) activation domain; an activation domain comprising four tandem copies of VP16; a VP64 activation domain; a p65 activation domain of NFκB (p65); an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase core domain of the human E1A-associated protein p300 (p300 HAT core activation domain). the transcriptional activator is selected from the group consisting of: a Herpes Simplex Virus Protein 16 (VP16) activation domain; an activation domain comprising four tandem copies of VP16; a VP64 activation domain; a p65 activation domain of NFκB (p65); an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase core domain of the human E1A-associated protein p300 (p300 HAT core activation domain), optionally wherein the transcriptional activator is a p65 transcriptional activator comprising the amino acid sequence of

(SEQ ID NO: 64)
DEFPTMVFPSGQISQASALAPAPPQVLPQAPAPAPAPAMVSALAQAPAP
VPVLAPGPPQAVAPPAPKPTQAGEGTLSEALLQLQFDDEDLGALLGNST
DPAVFTDLASVDNSEFQQLLNQGIPVAPHTTEPMLMEYPEAITRLVTGA
QRPPDPAPAPLGAPGLPNGLLSGDEDFSSIADMDFSALLSQISS.

Also provided herein, in various embodiments, is an isolated polynucleotide molecule comprising a nucleotide sequence encoding the modified ER-LBD or the chimeric protein provided herein.

Also provided herein, in various embodiments, is a heterologous construct comprising a promoter operatively linked to the polynucleotide molecule provided herein.

Also provided herein, in various embodiments, is a cell comprising the heterologous construct provided herein.

Also provided herein, in various embodiments, is a molecular switch for modulating transcription of a gene of interest, comprising: (a) the chimeric protein or a heterologous construct encoding the chimeric protein of claim 16, wherein the chimeric protein binds to a chimeric transcription factor-responsive (CTF-responsive) promoter operably linked to the gene of interest; and (b) a non-endogenous ligand, wherein binding of the non-endogenous ligand to the modified ER-LBD induces the chimeric protein to modulate transcription of the gene of interest, optionally wherein: (i) the non-endogenous ligand is selected from: 4-hydroxytamoxifen, N-desmethyltamoxifen, tamoxifen-N-oxide, tamoxifen, and endoxifen; (ii) the gene of interest encodes a polypeptide selected from the group consisting of: a cytokine, a chemokine, a homing molecule, a growth factor, a cell death regulator, a co-activation molecule, a tumor microenvironment modifier a, a receptor, a ligand, an antibody, a polynucleotide, a peptide, and an enzyme; (iii) the molecular switch of further comprises an additional construct comprising the CTF-responsive promoter operably linked to the gene of interest; (iv) the heterologous construct and the additional construct are comprised in a single vector; and/or (v) the heterologous construct is comprised in a first vector and the additional construct is comprised in a second vector.

Also provided herein, in various embodiments, is a method of modulating localization of a chimeric protein, comprising: (a) transforming a cell with a heterologous construct encoding the chimeric protein provided herein; and (b) inducing nuclear localization of the chimeric protein by contacting the transformed cell with a non-endogenous ligand, optionally wherein the method further comprises culturing the transformed cell under conditions suitable for expression of the chimeric protein prior to contacting the transformed cell with the non-endogenous ligand, and/or optionally wherein the heterologous construct and the additional construct are comprised in a single vector or the heterologous construct is comprised in a first vector and the additional construct is comprised in a second vector, and/or optionally wherein the non-endogenous ligand is selected from the group consisting of: 4-hydroxytamoxifen, N-desmethyltamoxifen, tamoxifen-N-oxide, tamoxifen, and endoxifen, and/or optionally wherein the non-endogenous ligand is administered at a concentration at which the non-endogenous ligand is substantially inactive on a wild-type estrogen receptor alpha of SEQ ID NO: 1.

Also provided herein, in some embodiments, are modified estrogen receptor ligand binding domains (ER-LBD) with improved sensitivity and/or selectivity for non-endogenous ligands, such as tamoxifen and metabolites thereof. Also provided herein, in some embodiments, are chimeric proteins including a modified ER-LBD as described herein, molecular switches, polynucleotides encoding the modified ER-LBD and chimeric protein as described herein, cells encoding the polynucleotides described herein or expressing the modified ER-LBD and chimeric protein as described herein, and methods of using the modified ER-LBD, chimeric protein, polynucleotide, molecular switch, or cells as described herein.

The modified ER-LBD and chimeric proteins (e.g., any of the polypeptides, the first polypeptide monomers, and/or the second polypeptide monomers of the inducible cell death systems herein) described herein have greater sensitivity to and/or selectivity for non-endogenous ligands (e.g., 4-hydroxytamoxifen, also referred to as “4-OHT”) as compared to ERT2. ERT2 is a ligand binding domain of ER which includes a G400V amino acid substitution, an M543A amino acid substitution, and an L544A amino acid substitution (see, e.g., SEQ ID NO: 2, SEQ ID NO: 3A). ERT2 may also include, in addition to G400V/M543A/L544A, a V595A amino acid substitution (see, e.g., SEQ ID NO: 3, SEQ ID NO: 3B). The average peak plasma concentration following a typical clinical dose of tamoxifen is in the nanomolar range (e.g., approximately 40 ng/ml). Furthermore, ERT2 may be responsive to endogenous ligands such as estradiol. Thus, the improved sensitivity to and/or selectivity for non-endogenous ligands of the modified ER-LBD and chimeric proteins including a modified ER-LBD allow for use of ER-based systems for suicide-switch mediated killing.

Provided herein is an inducible cell-death system comprising a polypeptide, wherein the polypeptide comprises a ligand binding domain and a cell death inducing domain, wherein the polypeptide is configured upon contact with a ligand of the ligand binding domain to generate a cell-death inducing signal in a cell in which the polypeptide is expressed, and wherein the ligand binding domain comprises a modified estrogen receptor ligand binding domain (ER-LBD) comprising an amino acid sequence corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions are with reference to one or more regions selected from: positions 343-354, positions 380-392, positions 404-463, and positions 517-540, and position 547 of SEQ ID NO: 1.

In some embodiments, the modified ER-LBD may further comprise yet other modifications, e.g., amino acid substitutions, deletions, and/or insertions (with reference to SEQ ID NO: 1). Such yet other modifications can be within or outside of positions 343-354, positions 380-392, positions 404-463, positions 517-540, and/or position 547, with reference to SEQ ID NO: 1. Such yet other modifications can be within or outside of outside of positions 283-594, with reference to SEQ ID NO: 1.

In some aspects, the polypeptide is or comprises a first polypeptide monomer and the inducible cell-death system further comprises a second polypeptide monomer, and wherein the first polypeptide monomer and the second polypeptide monomer each comprise a ligand binding domain and a cell death inducing domain, wherein the first polypeptide monomer and the second polypeptide monomer are configured to oligomerize with each other upon contact with a ligand of the ligand binding domain, thereby generating the cell-death inducing signal in a cell in which the first polypeptide monomer and the second polypeptide monomer are expressed.

In some aspects, the ligand binding domain of the first polypeptide monomer and the second polypeptide monomer each comprise a modified ER-LBD, wherein the modified ER-LBD comprises: (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions are selected independently for each of the first polypeptide monomer and the second polypeptide monomer with reference to one or more regions selected from: positions 343-354, positions 380-392, positions 404-463, and positions 517-540, and position 547 of SEQ ID NO: 1. In some aspects, the ligand binding domain of the first polypeptide monomer and the second polypeptide monomer comprise the same additional amino acid substitutions.

In some aspects, the modified ER-LBD has greater sensitivity to a non-endogenous ligand as compared to an ER-LBD comprising the amino acid sequence of SEQ ID NO: 2. In some aspects, the modified ER-LBD has greater sensitivity to a non-endogenous ligand as compared to an endogenous ligand as a result of the one or more additional amino acid substitutions. In some aspects, the modified ER-LBD has greater selectivity to a non-endogenous ligand as compared to an ER-LBD comprising the amino acid sequence of SEQ ID NO: 2.

In some aspects, the one or more additional amino acid substitutions of the polypeptide, the first polypeptide monomer (e.g., the polypeptide of an inducible cell death system including both a first and a second polypeptide monomer), and/or the second polypeptide monomer comprise an L391 substitution. In some aspects, the L391 substitution is L391V. In some aspects, the one or more additional amino acid substitutions of the polypeptide, the first polypeptide monomer, and/or the second polypeptide monomer comprise an N413D mutation. In some aspects, the one or more additional amino acid substitutions of the polypeptide, the first polypeptide monomer, and/or the second polypeptide monomer comprise an L391V substitution and an N413D mutation.

In some aspects, the one or more additional amino acid substitutions of the polypeptide, the first polypeptide monomer, and/or the second polypeptide monomer comprises an H524 substitution. In some aspects, the H524 substitution is an H524L substitution or an H524F substitution. In some aspects, the H524 substitution is an H524L substitution.

In some aspects, the H524 substitution is an H524F substitution.

In some aspects, the one or more additional amino acid substitutions of the polypeptide, the first polypeptide monomer, and/or the second polypeptide monomer comprises an M421L substitution.

In some aspects, the one or more additional amino acid substitutions of the polypeptide, the first polypeptide monomer, and/or the second polypeptide monomer comprises an S463P substitution.

In some aspects, the one or more additional amino acid substitutions of the polypeptide, the first polypeptide monomer, and/or the second polypeptide monomer comprises an M421L substitution and an S463P substitution.

In some aspects, the one or more additional amino acid substitutions of the polypeptide, the first polypeptide monomer, and/or the second polypeptide monomer comprises an L384M substitution.

In some aspects, the one or more additional amino acid substitutions of the polypeptide, the first polypeptide monomer, and/or the second polypeptide monomer comprises a L354I substitution.

In some aspects, the one or more additional amino acid substitutions of the polypeptide, the first polypeptide monomer, and/or the second polypeptide monomer comprises a Q414E substitution.

In some aspects, the one or more additional amino acid substitutions of the polypeptide, the first polypeptide monomer, and/or the second polypeptide monomer comprises a L354I substitution and a Q414E substitution.

In some aspects, the one or more additional amino acid substitutions of the polypeptide, the first polypeptide monomer, and/or the second polypeptide monomer comprises an L391V substitution, an N413D mutation, and an H524 substitution. In some aspects, the one or more additional amino acid substitutions of the polypeptide, the first polypeptide monomer, and/or the second polypeptide monomer comprises an L391V substitution, an N413D mutation, an H524 substitution, and an M421L substitution. In some aspects, the one or more additional amino acid substitutions of the polypeptide, the first polypeptide monomer, and/or the second polypeptide monomer comprises an L391V substitution, an N413D mutation, an H524 substitution, and an S463P substitution. In some aspects, the one or more additional amino acid substitutions of the polypeptide, the first polypeptide monomer, and/or the second polypeptide monomer comprises an L391V substitution, an N413D mutation, an H524 substitution, and an Q414E substitution. In some aspects, the one or more additional amino acid substitutions of the polypeptide, the first polypeptide monomer, and/or the second polypeptide monomer comprises an L391V substitution, an N413D mutation, an H524 substitution, and an L354I substitution.

In some aspects, the H524 substitution is an H524L substitution or an H524F substitution.

In some aspects, the modified estrogen receptor ligand binding domain (ER-LBD) comprises an amino acid sequence corresponding to a hormone binding domain of a reference human estrogen receptor sequence (SEQ ID NO: 1), wherein the modified ER-LBD comprises a G400V amino acid substitution, an M543A amino acid substitution, and an L544A amino acid substitution, with reference to SEQ ID NO: 1, and one or more additional amino acid substitutions, wherein the one or more additional amino acid substitutions are with reference to one or more regions selected from: positions 343-354, positions 380-392, positions 404-463, and positions 517-540, and position 547 of SEQ ID NO: 1, and wherein the modified ER-LBD has greater sensitivity and/or selectivity to a non-endogenous ligand as compared to an ER-LBD comprising the amino acid sequence of SEQ ID NO: 2, or as compared to an endogenous ligand as a result of the one or more additional amino acid substitutions. In some aspects, the modified ER-LBD further comprises a V595A amino acid substitution. In some aspects, the non-endogenous ligand is selected from: 4-hydroxytamoxifen, N-desmethyltamoxifen, tamoxifen-N-oxide, and endoxifen.

In some aspects, the one or more additional amino acid substitutions of the polypeptide, the first polypeptide monomer, and/or the second polypeptide monomer are at one or more positions of SEQ ID NO: 1 selected from: 343, 344, 345, 346, 347, 348, 349, 350, 351, 352, 354, 380, 384, 386, 387, 388, 389, 391, 392, 404, 407, 409, 413, 414, 417, 418, 420, 421, 422, 424, 428, 463, 517, 521, 522, 524, 525, 526, 527, 528, 533, 534, 536, 537, 538, 539, 540, and 547.

In some aspects, the one or more positions comprise position 343 of SEQ ID NO: 1. In some aspects, the amino acid substitution at position 343 of SEQ ID NO: 1 is selected from the group consisting of: M343F, M343I, M343L, and M343V.

In some aspects, the one or more positions comprise position 344 of SEQ ID NO: 1. In some aspects, the amino acid substitution at position 344 of SEQ ID NO: 1 is G344M.

In some aspects, the one or more positions comprise position 345 of SEQ ID NO: 1. In some aspects, the amino acid substitution at position 345 of SEQ ID NO: 1 is L345S.

In some aspects, the one or more positions comprise position 346 of SEQ ID NO: 1. In some aspects, the amino acid substitution at position 346 of SEQ ID NO: 1 is selected from the group consisting of: L346I, L346M, L346F, and L346V.

In some aspects, the one or more positions comprise position 347 of SEQ ID NO: 1. In some aspects, the amino acid substitution at position 347 of SEQ ID NO: 1 is selected from the group consisting of: T347D, T347E, T347F, T347I, T347K, T347L, T347M, T347N, T347Q, T347R, T347S, and T347V.

In some aspects, the one or more positions comprise position 348 of SEQ ID NO: 1. In some aspects, the amino acid substitution at position 348 of SEQ ID NO: 1 is N348K.

In some aspects, the one or more positions comprise position 349 of SEQ ID NO: 1. In some aspects, the amino acid substitution at position 349 of SEQ ID NO: 1 is selected from the group consisting of: L349I, L349M, L349F, and L349V.

In some aspects, the one or more positions comprise position 350 of SEQ ID NO: 1. In some aspects, the amino acid substitution at position 350 of SEQ ID NO: 1 is selected from the group consisting of: A350F, A350I, A350L, A350M and A350V.

In some aspects, the one or more positions comprise position 351 of SEQ ID NO: 1. In some aspects, the amino acid substitution at position 351 of SEQ ID NO: 1 is selected from the group consisting of: D351E, D351F, D351I, D351L, D351M, D351N, D351Q, and D351V.

In some aspects, the one or more positions comprise position 352 of SEQ ID NO: 1. In some aspects, the amino acid substitution at position 352 of SEQ ID NO: 1 is R352K.

In some aspects, the one or more positions comprise position 354 of SEQ ID NO: 1. In some aspects, the amino acid substitution at position 354 of SEQ ID NO: 1 is selected from the group consisting of: L354I, L354M, L354F, and L354V.

In some aspects, the one or more positions comprise position 380 of SEQ ID NO: 1. In some aspects, the amino acid substitution at position 380 of SEQ ID NO: 1 is E380Q.

In some aspects, the one or more positions comprise position 384 of SEQ ID NO: 1. In some aspects, the amino acid substitution at position 384 of SEQ ID NO: 1 is selected from the group consisting of: L384I, L384M, L384F, and L384V.

In some aspects, the one or more positions comprise position 386 of SEQ ID NO: 1. In some aspects, the amino acid substitution at position 386 of SEQ ID NO: 1 is I386V.

In some aspects, the one or more positions comprise position 387 of SEQ ID NO: 1. In some aspects, the amino acid substitution at position 387 of SEQ ID NO: 1 is selected from the group consisting of: L387I, L387M, L387F, and L387V.

In some aspects, the one or more positions comprise position 388 of SEQ ID NO: 1. In some aspects, the amino acid substitution at position 388 of SEQ ID NO: 1 is selected from the group consisting of: M388I, M388L, and M388F.

In some aspects, the one or more positions comprise position 389 of SEQ ID NO: 1. In some aspects, the amino acid substitution at position 389 of SEQ ID NO: 1 is I389M.

In some aspects, the one or more positions comprise position 391 of SEQ ID NO: 1. In some aspects, the amino acid substitution at position 391 of SEQ ID NO: 1 is selected from the group consisting of: L391I, L391M, L391F, and L391V.

In some aspects, the one or more positions comprise position 392 of SEQ ID NO: 1. In some aspects, the amino acid substitution at position 392 of SEQ ID NO: 1 is V392M.

In some aspects, the one or more positions comprise position 404 of SEQ ID NO: 1. In some aspects, the amino acid substitution at position 404 of SEQ ID NO: 1 is selected from the group consisting of: F404I, F404L, F404M, and F404V.

In some aspects, the one or more positions comprise position 407 of SEQ ID NO: 1. In some aspects, the amino acid substitution at position 407 of SEQ ID NO: 1 is N407D.

In some aspects, the one or more positions comprise position 409 of SEQ ID NO: 1. In some aspects, the amino acid substitution at position 409 of SEQ ID NO: 1 is L409V.

In some aspects, the one or more positions comprise position 413 of SEQ ID NO: 1. In some aspects, the amino acid substitution at position 413 of SEQ ID NO: 1 is N413D.

In some aspects, the one or more positions comprise position 414 of SEQ ID NO: 1. In some aspects, the amino acid substitution at position 414 of SEQ ID NO: 1 is Q414E.

In some aspects, the one or more positions comprise position 417 of SEQ ID NO: 1. In some aspects, the amino acid substitution at position 417 of SEQ ID NO: 1 is C417S.

In some aspects, the one or more positions comprise position 418 of SEQ ID NO: 1. In some aspects, the amino acid substitution at position 418 of SEQ ID NO: 1 is selected from the group consisting of: V418I, V418L, V418M, and V418F.

In some aspects, the one or more positions comprise position 420 of SEQ ID NO: 1. In some aspects, the amino acid substitution at position 420 of SEQ ID NO: 1 is selected from the group consisting of: G420I, G420M, G420F, and G420V.

In some aspects, the one or more positions comprise position 421 of SEQ ID NO: 1. In some aspects, the amino acid substitution at position 421 of SEQ ID NO: 1 is selected from the group consisting of: M421I, M421L, M421F, and M421V.

In some aspects, the one or more positions comprise position 422 of SEQ ID NO: 1. In some aspects, the amino acid substitution at position 422 of SEQ ID NO: 1 is V422I.

In some aspects, the one or more positions comprise position 424 of SEQ ID NO: 1. In some aspects, the amino acid substitution at position 424 of SEQ ID NO: 1 is selected from the group consisting of: 1424L, I424M, 1424F, and I424V.

In some aspects, the one or more positions comprise position 428 of SEQ ID NO: 1. In some aspects, the amino acid substitution at position 428 of SEQ ID NO: 1 is selected from the group consisting of: L428I, L428M, L428F, and L428V.

In some aspects, the one or more positions comprise position 463 of SEQ ID NO: 1. In some aspects, the amino acid substitution at position 463 of SEQ ID NO: 1 is S463P.

In some aspects, the one or more positions comprise position 517 of SEQ ID NO: 1. In some aspects, the amino acid substitution at position 517 of SEQ ID NO: 1 is M517A.

In some aspects, the one or more positions comprise position 521 of SEQ ID NO: 1. In some aspects, the amino acid substitution at position 521 of SEQ ID NO: 1 is selected from the group consisting of: G521A, G521F, G521I, G521L, G521M, and G521V.

In some aspects, the one or more positions comprise position 522 of SEQ ID NO: 1, In some aspects, the amino acid substitution at position 522 of SEQ ID NO: 1 is selected from the group consisting of: M522I, M522L, and M522V.

In some aspects, the one or more positions comprise position 524 of SEQ ID NO: 1. In some aspects, the amino acid substitution at position 524 of SEQ ID NO: 1 is selected from the group consisting of: H524A, H524I, H524L, H524F, and H524V.

In some aspects, the one or more positions comprise position 525 of SEQ ID NO: 1. In some aspects, the amino acid substitution at position 525 of SEQ ID NO: 1 is selected from the group consisting of: L525F, L525I, L525M, L525N, L525Q, L525S, L525T, and L525V.

In some aspects, the one or more positions comprise position 526 of SEQ ID NO: 1. In some aspects, the amino acid substitution at position 526 of SEQ ID NO: 1 is Y526L.

In some aspects, the one or more positions comprise position 527 of SEQ ID NO: 1. In some aspects, the amino acid substitution at position 527 of SEQ ID NO: 1 is S527N.

In some aspects, the one or more positions comprise position 528 of SEQ ID NO: 1. In some aspects, the amino acid substitution at position 528 of SEQ ID NO: 1 is selected from the group consisting of: M528F, M528I, and M528V.

In some aspects, the one or more positions comprise position 533 of SEQ ID NO: 1. In some aspects, the amino acid substitution at position 533 of SEQ ID NO: 1 is selected from the group consisting of: V533F and V533W.

In some aspects, the one or more positions comprise position 534 of SEQ ID NO: 1. In some aspects, the amino acid substitution at position 534 of SEQ ID NO: 1 is selected from the group consisting of: V534Q and V534R.

In some aspects, the one or more positions comprise position 536 of SEQ ID NO: 1. In some aspects, the amino acid substitution at position 536 of SEQ ID NO: 1 is selected from the group consisting of: L536F, and L536M, L536R, and L536Y.

In some aspects, the one or more positions comprise position 537 of SEQ ID NO: 1. In some aspects, the amino acid substitution at position 537 of SEQ ID NO: 1 is selected from the group consisting of: Y537E and Y537S.

In some aspects, the one or more positions comprise position 538 of SEQ ID NO: 1. In some aspects, the amino acid substitution at position 538 of SEQ ID NO: 1 is selected from the group consisting of: D538G and D538K.

In some aspects, the one or more positions comprise position 539 of SEQ ID NO: 1. In some aspects, the amino acid substitution at position 539 of SEQ ID NO: 1 is selected from the group consisting of: L539A and L539R.

In some aspects, the one or more positions comprise position 540 of SEQ ID NO: 1. In some aspects, the amino acid substitution at position 540 of SEQ ID NO: 1 is selected from the group consisting of: L540A and L540F.

In some aspects, the one or more positions comprise position 547 of SEQ ID NO: 1. In some aspects, the amino acid substitution at position 547 of SEQ ID NO: 1 is H547A.

In some aspects, the one or more additional amino acid substitutions are two amino acid substitutions. In some aspects, each of the two amino acid substitutions are at a position of SEQ ID NO: 1 selected from: 343, 345, 347, 348, 351, 354, 384, 387, 388, 389, 391, 392, 404, 418, 421, 521, 524, and 525. In some aspects, the two amino acid substitutions are at positions 345 and 348 of SEQ ID NO: 1 and wherein the amino acid substitution at position 345 of SEQ ID NO: 1 is L345S and the amino acid substitution at position 348 of SEQ ID NO: 1 is N348K. In some aspects, the two amino acid substitutions are at positions 384 and 389 of SEQ ID NO: 1 and wherein the amino acid substitution at position 384 of SEQ ID NO: 1 is L384M and the amino acid substitution at position 389 of SEQ ID NO: 1 is 1389M. In some aspects, the two amino acid substitutions are at positions 421 and 392 of SEQ ID NO: 1 and wherein the amino acid substitution at position 421 of SEQ ID NO: 1 is M421I and the amino acid substitution at position 392 of SEQ ID NO: 1 is V392M. In some aspects, the two amino acid substitutions are at positions 354 and 391 of SEQ ID NO: 1 and wherein the amino acid substitution at position 354 of SEQ ID NO: 1 is L354I and the amino acid substitution at position 391 of SEQ ID NO: 1 is L391F. In some aspects, the two amino acid substitutions are at positions 354 and 384 of SEQ ID NO: 1 and wherein the amino acid substitution at position 354 of SEQ ID NO: 1 is L354I and the amino acid substitution at position 384 of SEQ ID NO: 1 is L384M. In some aspects, the two amino acid substitutions are at positions 354 and 387 of SEQ ID NO: 1 and wherein the amino acid substitution at position 354 of SEQ ID NO: 1 is L354I and the amino acid substitution at position 387 of SEQ ID NO: 1 is L387M. In some aspects, the two amino acid substitutions are at positions 387 and 391 and wherein the amino acid substitution at position 387 of SEQ ID NO: 1 is L387M and the amino acid substitution at position 391 of SEQ ID NO: 1 is L391F. In some aspects, the two amino acid substitutions are at positions 384 and 387 of SEQ ID NO: 1 and wherein the amino acid substitution at position 384 of SEQ ID NO: 1 is L384M and the amino acid substitution at position 387 of SEQ ID NO: 1 is L387M. In some aspects, the two amino acid substitutions are at positions 384 and 391 of SEQ ID NO: 1 and wherein the amino acid substitution at position 384 of SEQ ID NO: 1 is L384M and the amino acid substitution at position 391 of SEQ ID NO: 1 is L391F.

In some aspects, the one or more additional amino acid substitutions are three amino acid substitutions. In some aspects, each of the three amino acid substitutions are at a position of SEQ ID NO: 1 selected from: 343, 347, 351, 354, 388, 391, 404, 414, 418, 463, 521, 524, and 525. In some aspects, the three amino acid substitutions are at positions 354, 384, and 391 of SEQ ID NO: 1 and wherein the amino acid substitution at position 354 of SEQ ID NO: 1 is L354I, the amino acid substitution at position 384 of SEQ ID NO: 1 is L384M, and the amino acid substitution at position 391 of SEQ ID NO: 1 is L391F. In some aspects, the three amino acid substitutions are at positions 414, 463, and 524 of SEQ ID NO: 1 and wherein the amino acid substitution at position 414 of SEQ ID NO: 1 is Q414E, the amino acid substitution at position 463 of SEQ ID NO: 1 is S463P, and the amino acid substitution at position 524 of SEQ ID NO: 1 is H524L.

In some aspects, the one or more additional amino acid substitutions are four amino acid substitutions. In some aspects, each of the four amino acid substitutions are at a position of SEQ ID NO: 1 selected from: 343, 347, 351, 354, 384, 388, 391, 404, 413, 418, 463, 521, 524, and 525. In some aspects, the four amino acid substitutions are at positions 354, 384, 391, and 418 of SEQ ID NO: 1 and wherein the amino acid substitution at position 354 of SEQ ID NO: 1 is L354I, the amino acid substitution at position 384 of SEQ ID NO: 1 is L384M, the amino acid substitution at position 391 of SEQ ID NO: 1 is L391F, and the amino acid substitution at position 418 of SEQ ID NO: 1 is V418I. In some aspects, the four amino acid substitutions are at positions 343, 388, 521, and 404 of SEQ ID NO: 1 and wherein the amino acid substitution at position 343 of SEQ ID NO: 1 is M343I, the amino acid substitution at position 388 of SEQ ID NO: 1 is M388I, the amino acid substitution at position 521 of SEQ ID NO: 1 is G521I, and the amino acid substitution at position 404 of SEQ ID NO: 1 is F404L. In some aspects, the four amino acid substitutions are at positions 524, 347, 351, and 525 of SEQ ID NO: 1 and wherein the amino acid substitution at position 524 of SEQ ID NO: 1 is H524V, the amino acid substitution at position 347 of SEQ ID NO: 1 is T347R, the amino acid substitution at position 351 of SEQ ID NO: 1 is D351Q, and the amino acid substitution at position 525 of SEQ ID NO: 1 is L525N. In some aspects, the four amino acid substitutions are at positions 354, 384, 391, and 463 of SEQ ID NO: 1 and wherein the amino acid substitution at position 354 of SEQ ID NO: 1 is L354I, the amino acid substitution at position 384 of SEQ ID NO: 1 is L384M, the amino acid substitution at position 391 of SEQ ID NO: 1 is L391V, and the amino acid substitution at position 463 of SEQ ID NO: 1 is S463P. In some aspects, the four amino acid substitutions are at positions 384, 391, 413, and 524 of SEQ ID NO: 1 and wherein the amino acid substitution at position 384 of SEQ ID NO: 1 is L384M, the amino acid substitution at position 391 of SEQ ID NO: 1 is L391V, the amino acid substitution at position 413 of SEQ ID NO: 1 is N413D, and the amino acid substitution at position 524 of SEQ ID NO: 1 is H524F.

In some aspects, the one or more additional amino acid substitutions are five amino acid substitutions. In some aspects, each of the five amino acid substitutions are at a position of SEQ ID NO: 1 selected from: 354, 384, 391, 409, 413, 414, 421, 463, and 524. In some aspects, the five amino acid substitutions are at positions 384, 409, 413, 463, and 524 of SEQ ID NO: 1 and wherein the amino acid substitution at position 384 of SEQ ID NO: 1 is L384M, the amino acid substitution at position 409 of SEQ ID NO: 1 is L409V, the amino acid substitution at position 413 of SEQ ID NO: 1 is N413D, the amino acid substitution at position 463 of SEQ ID NO: 1 is S463P, and the amino acid substitution at position 524 of SEQ ID NO: 1 is H524L. In some aspects, the five amino acid substitutions are at positions 391, 413, 414, 463, and 524 of SEQ ID NO: 1 and wherein the amino acid substitution at position 391 of SEQ ID NO: 1 is L391V, the amino acid substitution at position 413 of SEQ ID NO: 1 is N413D, the amino acid substitution at position 414 of SEQ ID NO: 1 is Q414E, the amino acid substitution at position 463 of SEQ ID NO: 1 is S463P, and the amino acid substitution at position 524 of SEQ ID NO: 1 is H524F. In some aspects, the five amino acid substitutions are at positions 391, 414, 421, 463, and 524 of SEQ ID NO: 1 and wherein the amino acid substitution at position 391 of SEQ ID NO: 1 is L391V, the amino acid substitution at position 414 of SEQ ID NO: 1 is Q414E, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524F. In some aspects, the five amino acid substitutions are at positions 354, 409, 413, 421, and 524 of SEQ ID NO: 1 and wherein the amino acid substitution at position 354 of SEQ ID NO: 1 is L354I, the amino acid substitution at position 409 of SEQ ID NO: 1 is L409V, the amino acid substitution at position 413 of SEQ ID NO: 1 is N413D, the amino acid substitution at position 421 of SEQ ID NO: 1 is M421L, and the amino acid substitution at position 524 of SEQ ID NO: 1 is H524L. In some aspects, the five amino acid substitutions are at positions 354, 409, 421, 463, and 524 of SEQ ID NO: 1 and wherein the amino acid substitution at position 354 of SEQ ID NO: 1 is L354I, the amino acid substitution at position 409 of SEQ ID NO: 1 is L409V, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524L.

In some aspects, the one or more additional amino acid substitutions are six amino acid substitutions. In some aspects, each of the six amino acid substitutions are at a position of SEQ ID NO: 1 selected from: 354, 384, 391, 409, 413, 414, 421, 463, and 524. In some aspects, the six amino acid substitutions are at positions 384, 391, 413, 421, 463, and 524 of SEQ ID NO: 1 and wherein the amino acid substitution at position 384 of SEQ ID NO: 1 is L384M, the amino acid substitution at position 391 of SEQ ID NO: 1 is L391V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524L. In some aspects, the six amino acid substitutions are at positions 409, 413, 414, 421, 463, and 524 of SEQ ID NO: 1 and wherein the amino acid substitution at position 409 of SEQ ID NO: 1 is L409V, the amino acid substitution at position 413 of SEQ ID NO: 1 is N413D, the amino acid substitution at position 414 of SEQ ID NO: 1 is Q414E, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524L. In some aspects, the six amino acid substitutions are at positions 354, 391, 409, 413, 414, and 524 of SEQ ID NO: 1 and wherein the amino acid substitution at position 354 of SEQ ID NO: 1 is L354I, the amino acid substitution at position 391 of SEQ ID NO: 1 is L391V, the amino acid substitution at position 409 of SEQ ID NO: 1 is L409V, the amino acid substitution at position 413 of SEQ ID NO: 1 is N413D, the amino acid substitution at position 414 of SEQ ID NO: 1 is Q414E, and the amino acid substitution at position 524 of SEQ ID NO: 1 is H524L.

In some aspects, the one or more additional amino acid substitutions are seven amino acid substitutions. In some aspects, each of the seven amino acid substitutions are at a position of SEQ ID NO: 1 selected from: 354, 384, 391, 409, 413, 414, 421, 463, 517, and 524. In some aspects, the seven amino acid substitutions are at positions 354, 384, 409, 413, 421, 463, and 524 of SEQ ID NO: 1 and wherein the amino acid substitution at position 354 of SEQ ID NO: 1 is L354I, the amino acid substitution at position 384 of SEQ ID NO: 1 is L384M, the amino acid substitution at position 409 of SEQ ID NO: 1 is L409V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524F. In some aspects, the seven amino acid substitutions are at positions 354, 391, 413, 421, 463, 517, and 524 of SEQ ID NO: 1 and wherein the amino acid substitution at position 354 of SEQ ID NO: 1 is L354I, the amino acid substitution at position 391 of SEQ ID NO: 1 is L391V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, the amino acid substitution at position 517 of SEQ ID NO: 1 is M517A, and the amino acid substitution at position 524 of SEQ ID NO: 1 is H524L. In some aspects, the seven amino acid substitutions are at positions 354, 391, 413, 414, 421, 517, and 524 of SEQ ID NO: 1 and wherein the amino acid substitution at position 354 of SEQ ID NO: 1 is L354I, the amino acid substitution at position 391 of SEQ ID NO: 1 is L391V, the amino acid substitution at position 413 of SEQ ID NO: 1 is N413D, the amino acid substitution at position 414 of SEQ ID NO: 1 is Q414E, the amino acid substitution at position 421 of SEQ ID NO: 1 is M421L, the amino acid substitution at position 517 of SEQ ID NO: 1 is M517A, and the amino acid substitution at position 524 of SEQ ID NO: 1 is H524F.

In some aspects, the one or more additional amino acid substitutions are eight amino acid substitutions. In some aspects, the eight amino acid substitutions are at positions 384, 391, 409, 413, 421, 463, 517, and 524 of SEQ ID NO: 1 and wherein the amino acid substitution at position 384 of SEQ ID NO: 1 is L384M, the amino acid substitution at position 391 of SEQ ID NO: 1 is L391V, the amino acid substitution at position 409 of SEQ ID NO: 1 is L409V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, the amino acid substitution at position 517 of SEQ ID NO: 1 is M517A, and the amino acid substitution at position 524 of SEQ ID NO: 1 is H524F.

In some aspects, the one or more additional amino acid substitutions of the polypeptide, the first polypeptide monomer, and/or the second polypeptide monomer comprises an N413D mutation, an H524 substitution, and an S463P substitution.

In some aspects, the one or more additional amino acid substitutions of the polypeptide, the first polypeptide monomer, and/or the second polypeptide monomer comprise an L391V substitution, an L409V substitution, an Q414E substitution, an N413D substitution, an S463P substitution, an M517A substitution, and an H524L substitution. In some aspects, the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 90 or 103.

In some aspects, the one or more additional amino acid substitutions of the polypeptide, the first polypeptide monomer, and/or the second polypeptide monomer comprise an L409V substitution, an N413D substitution, an S463P substitution, an M421L substitution, an L384M substitution, and an H524L substitution. In some aspects, the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 91 or 104.

In some aspects, the one or more additional amino acid substitutions of the polypeptide, the first polypeptide monomer, and/or the second polypeptide monomer comprise an L391V substitution, an L409V substitution, an N413D substitution, an S463P substitution, an M517A substitution, an M421L substitution, an L354I substitution, and an H524L substitution. In some aspects, the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 92 or 105.

In some aspects, the one or more additional amino acid substitutions of the polypeptide, the first polypeptide monomer, and/or the second polypeptide monomer comprise an L391V substitution, an Q414E substitution, an N413D substitution, an S463P substitution, an M421L substitution, an L354I substitution, an L384M substitution, and an H524L substitution. In some aspects, the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 93 or 106.

In some aspects, the one or more additional amino acid substitutions of the polypeptide, the first polypeptide monomer, and/or the second polypeptide monomer comprise an L391V substitution, an L409V substitution, an N413D substitution, an S463P substitution, an M517A substitution, an M421L substitution, and an H524L substitution. In some aspects, the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 94 or 107.

In some aspects, the one or more additional amino acid substitutions of the polypeptide, the first polypeptide monomer, and/or the second polypeptide monomer comprise an L391V substitution, an L409V substitution, an Q414E substitution, an N413D substitution, an S463P substitution, an L354I substitution, and an H524L substitution. In some aspects, the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 95 or 108.

In some aspects, the one or more additional amino acid substitutions of the polypeptide, the first polypeptide monomer, and/or the second polypeptide monomer comprise an L391V substitution, an L409V substitution, an N413D substitution, an S463P substitution, an M421L substitution, an L354I substitution, an L384M substitution, and an H524L substitution. In some aspects, the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 96 or 109.

In some aspects, the one or more additional amino acid substitutions of the polypeptide, the first polypeptide monomer, and/or the second polypeptide monomer comprise an L391V substitution, an Q414E substitution, an N413D substitution, an S463P substitution, an M517A substitution, an M421L substitution, an L354I substitution, and an H524L substitution. In some aspects, the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 97 or 110.

In some aspects, the one or more additional amino acid substitutions of the polypeptide, the first polypeptide monomer, and/or the second polypeptide monomer comprise an L391V substitution, an N413D substitution, an S463P substitution, an M517A substitution, an L384M substitution, and an H524L substitution. In some aspects, the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 98 or 111.

In some aspects, the one or more additional amino acid substitutions of the polypeptide, the first polypeptide monomer, and/or the second polypeptide monomer comprise an L391V substitution, an L409V substitution, an N413D substitution, an S463P substitution, an M517A substitution, and an H524L substitution. In some aspects, the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 99 or 112.

In some aspects, the one or more additional amino acid substitutions of the polypeptide, the first polypeptide monomer, and/or the second polypeptide monomer comprise an N413D substitution, an S463P substitution, an L354I substitution, an L384M substitution, and an H524L substitution. In some aspects, the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 100 or 113.

In some aspects, the one or more additional amino acid substitutions of the polypeptide, the first polypeptide monomer, and/or the second polypeptide monomer comprise an N413D substitution, an S463P substitution, an M421L substitution, an L354I substitution, and an H524L substitution. In some aspects, the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 101 or 114.

Also provided herein is an inducible cell death system comprising a first polypeptide and a second polypeptide monomer, wherein the first and the second polypeptide monomers each comprise a ligand binding domain and a cell death inducing domain, wherein the first and the second polypeptide monomers are configured to oligomerize upon contact with a ligand of the ligand binding domain, thereby generating a cell-death inducing signal in a cell in which the first and the second polypeptide monomers are expressed, and wherein the ligand binding domain comprises a modified estrogen receptor ligand binding domain (ER-LBD) comprising an amino acid sequence corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) additional amino acid substitutions, wherein the additional amino acid substitutions comprise, with reference to SEQ ID NO: 1: (i) an L384M substitution, an L391V substitution, a N413D substitution, an M421L substitution, a S463P substitution, and a H524L substitution, (ii) an L391V substitution, a N413D substitution, a Q414E substitution, a S463P substitution, and a H524F substitution, (iii) an L354I substitution, a L391V substitution, a N413D substitution, a Q414E substitution, a M421L substitution, a M517A substitution, and a H524F substitution, or (iv) an L354I substitution, a L391V substitution, a L409V substitution, a N413D substitution, a Q414E substitution, and a H524L substitution.

In some aspects, the ligand is a non-endogenous ligand. Exemplary non-endogenous ligands are provided herein. In some aspects, the non-endogenous ligand is selected from: 4-hydroxytamoxifen, N-desmethyltamoxifen, tamoxifen-N-oxide, and endoxifen. In some aspects, the non-endogenous ligand comprises a tamoxifen metabolite. In some aspects, the non-endogenous ligand is endoxifen. In some aspects, the first polypeptide monomer and the second polypeptide monomer are capable of oligomerization and/or generating the cell-death inducing signal at a concentration of 0.25 nM Endoxifen or less and/or at a concentration of 0.04 nM 4-OHT or less. In some aspects, the first polypeptide monomer and the second polypeptide monomer are capable of oligomerization and/or generating the cell-death inducing signal at a concentration of 2.5 nM Endoxifen or less and/or at a concentration of 0.4 nM 4-OHT or less. In some aspects, the first polypeptide monomer and the second polypeptide monomer are capable of oligomerization and/or generating the cell-death inducing signal at a concentration of at least 0.001 pM of 4-OHT. In some aspects, the first polypeptide monomer and the second polypeptide monomer are capable of oligomerization and/or generating the cell-death inducing signal at a concentration of at least 0.01 pM of 4-OHT.

In some aspects, the cell death-inducing domain is derived from a protein selected from: a caspase (e.g., any one of caspase 1-11, such as caspase 3, caspase 6, caspase 7, caspase 8, caspase 9), Diphtheria toxin fragment A (DTA), Bax, Bak, Bok, Bad, Bcl-Xs, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B1, and Purine nucleoside phosphorylase. In some aspects, the cell death-inducing domain comprises a caspase domain or a derivative or a functional fragment thereof. In some aspects, the caspase is selected from, e.g., any one of caspases 1-11, such as caspase 3, caspase 6, caspase 7, caspase 8, caspase 9, or derivatives or functional fragments thereof, respectively. In some aspects, the caspase is caspase 9, or a functional fragment thereof. In some aspects, the cell death-inducing domain comprises the Caspase 9 derived amino acid sequence of SEQ ID NO: 48 or 125. In some aspects, the caspase domain or derivative or functional fragment thereof, e.g., inducible Casp-9, does not comprise a Caspase Activation and Recruitment Domain (CARD) domain sequence.

In some aspects, the cell death-inducing domain is a transcription factor comprising a nucleic acid-binding domain and a transcriptional effector domain, wherein the transcription factor is configured to generate a cell-death inducing signal by inducing expression of: a caspase domain or derivative or functional fragment thereof, optionally wherein the caspase (e.g., any one of caspases 1-11) is selected from caspase 3, caspase 6, caspase 7, caspase 8, caspase 9, or derivatives or functional fragments thereof, respectively, Diphtheria toxin fragment A (DTA), Bax, Bak, Bok, Bad, Bcl-Xs, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B1, or Purine nucleoside phosphorylase.

Also provided herein is a modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an N413D substitution, an S463P substitution, an L354I substitution, an L384M substitution, and an H524L substitution, with reference to SEQ ID NO: 1.

Also provided herein is a modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an N413D substitution, an S463P substitution, an M421L substitution, an L354I substitution, and an H524L substitution, with reference to SEQ ID NO: 1.

Also provided herein is a modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L409V substitution, an N413D substitution, an S463P substitution, an M421L substitution, an L384M substitution, and an H524L substitution, with reference to SEQ ID NO: 1.

Also provided herein is a modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an L409V substitution, an Q414E substitution, an N413D substitution, an S463P substitution, an M517A substitution, and an H524L substitution, with reference to SEQ ID NO: 1.

Also provided herein is a modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an L409V substitution, an N413D substitution, an S463P substitution, an M517A substitution, an M421L substitution, an L354I substitution, and an H524L substitution, with reference to SEQ ID NO: 1.

Also provided herein is a modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an Q414E substitution, an N413D substitution, an S463P substitution, an M421L substitution, an L354I substitution, an L384M substitution, and an H524L substitution, with reference to SEQ ID NO: 1.

Also provided herein is a modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an L409V substitution, an N413D substitution, an S463P substitution, an M517A substitution, an M421L substitution, and an H524L substitution, with reference to SEQ ID NO: 1.

Also provided herein is a modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an L409V substitution, an Q414E substitution, an N413D substitution, an S463P substitution, an L354I substitution, and an H524L substitution, with reference to SEQ ID NO: 1.

Also provided herein is a modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an L409V substitution, an N413D substitution, an S463P substitution, an M421L substitution, an L354I substitution, an L384M substitution, and an H524L substitution, with reference to SEQ ID NO: 1.

Also provided herein is a modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an Q414E substitution, an N413D substitution, an S463P substitution, an M517A substitution, an M421L substitution, an L354I substitution, and an H524L substitution, with reference to SEQ ID NO: 1.

Also provided herein is a modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an N413D substitution, an S463P substitution, an M517A substitution, an L384M substitution, and an H524L substitution, with reference to SEQ ID NO: 1.

Also provided herein is a modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an L409V substitution, an N413D substitution, an S463P substitution, an M517A substitution, and an H524L substitution, with reference to SEQ ID NO: 1.

Also provided herein is a modified ER-LBD comprising an amino acid sequence at least 80%, 85%, 90%, 95%, 97.5%, 98%, 99%, or 100% identical to any one of SEQ ID Nos: 90-114.

In some embodiments of the modified ER-LBD, the modified ER-LBD has greater sensitivity and/or selectivity to a non-endogenous ligand as compared to an ER-LBD comprising the amino acid sequence of SEQ ID NO: 2, or as compared to an endogenous ligand as a result of the one or more additional amino acid substitutions. In some embodiments, the non-endogenous ligand is selected from the group consisting of: 4-hydroxytamoxifen, N-desmethyltamoxifen, tamoxifen-N-oxide, tamoxifen, and endoxifen. In some embodiments, the endogenous ligand is estradiol. In some embodiments, the modified ER-LBD further comprises a V595A amino acid substitution.

Also provided herein is a chimeric protein comprising a polypeptide of interest fused to a modified ER-LBD as described herein. In some aspects, the polypeptide of interest comprises a nucleic acid binding domain. In some aspects, the nucleic acid binding domain comprises a zinc finger domain. In some aspects, the nucleic acid binding domain comprises a zinc finger domain. In some aspects, the zinc finger domain comprises the sequence MSRPGERPFQCRICMRNFSNMSNLTRHTRTHTGEKPFQCRICMRNFSDRSVLRRH LRTHTGSQKPFQCRICMRNFSDPSNLARHTRTHTGEKPFQCRICMRNFSDRSSLRR HLRTHTGSQKPFQCRICMRNFSQSGTLHRHTRTHTGEKPFQCRICMRNFSQRPNLT RHLRTHLRGS (SEQ ID NO: 62). In some aspects, the zinc finger domain comprises the sequence as set forth in SEQ ID NO: 57 or SEQ ID NO: 84. In some aspects, the chimeric protein comprises a chimeric transcription factor, wherein the polypeptide of interest comprises a nucleic acid binding domain and a transcriptional modulator domain. In some aspects, the transcriptional modular domain is a transcriptional activator. In some aspects, the transcriptional activator is selected from: a Herpes Simplex Virus Protein 16 (VP16) activation domain; an activation domain comprising four tandem copies of VP16; a VP64 activation domain; a p65 activation domain of NFκB (p65); an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase core domain of the human E1A-associated protein p300 (p300 HAT core activation domain). In some aspects, the transcriptional modular domain is a p65 transcriptional activator comprising the amino acid sequence of

(SEQ ID NO: 64)
DEFPTMVFPSGQISQASALAPAPPQVLPQAPAPAPAPAMVSALAQAPAP
VPVLAPGPPQAVAPPAPKPTQAGEGTLSEALLQLQFDDEDLGALLGNST
DPAVFTDLASVDNSEFQQLLNQGIPVAPHTTEPMLMEYPEAITRLVTGA
QRPPDPAPAPLGAPGLPNGLLSGDEDFSSIADMDFSALLSQISS.

Also provided for herein is an isolated polynucleotide comprising a nucleotide sequence encoding a modified ER-LBD as described herein. Also provided herein is an isolated polynucleotide comprising a nucleotide sequence encoding a chimeric protein, a polypeptide, a first polypeptide monomer, and/or a second polypeptide monomer as described herein.

Also provided for herein is a heterologous construct comprising a promoter operatively linked to a polynucleotide as described herein.

Also provided for herein is a plasmid or vector comprising a heterologous construct as described herein.

Also provided for herein is a cell comprising a heterologous construct as described herein or a plasmid or vector as described herein.

Also provided for herein is a molecular switch for generating a cell-death inducing signal in a cell, comprising: (a) an inducible cell death system described herein, an isolated polynucleotide described herein, a heterologous construct described herein, a plasmid or vector described herein, or a cell described herein, wherein the inducible cell death system is capable of generating a cell-death inducing signal in the cell; and (b) a non-endogenous ligand, wherein binding of the non-endogenous ligand to the modified ER-LBD generates the cell-death inducing signal in the cell.

Also provided for herein is a molecular switch for generating a cell-death inducing signal in a cell, comprising: (a) an inducible cell death system described herein, an isolated polynucleotide described herein, a heterologous construct described herein, a plasmid or vector described herein, or a cell described herein, wherein the inducible cell death system is capable of generating a cell-death inducing signal in the cell upon oligomerization of a first and a second polypeptide monomers; and (b) a non-endogenous ligand, wherein binding of the non-endogenous ligand to the modified ER-LBD induces oligomerization of the first and the second polypeptide monomers, thereby generating the cell-death inducing signal in the cell.

Exemplary non-endogenous ligands are provided herein. In some aspects, the non-endogenous ligand is selected from: 4-hydroxytamoxifen, N-desmethyltamoxifen, tamoxifen-N-oxide, and endoxifen.

Also provided for herein is a method of inducing oligomerization of a chimeric protein comprising: transforming a cell with (i) a heterologous construct encoding any one of the inducible cell death systems described herein, an isolated polynucleotide described herein, a heterologous construct described herein, a plasmid or vector described herein, or a cell described herein, wherein the inducible cell death system is capable of generating a cell-death inducing signal in the cell upon oligomerization of a first and a second polypeptide monomers, and (ii) contacting the transformed cell with a non-endogenous ligand of the modified estrogen receptor ligand binding domain (ER-LBD).

Also provided for herein is a method of modulating transcription of a gene of interest, comprising: transforming a cell with (i) a heterologous construct encoding a chimeric protein as described herein and (ii) a target expression cassette comprising a chimeric transcription factor-responsive (CTF-responsive) promoter operably linked to the gene of interest, and inducing the chimeric protein to modulate transcription of the gene of interest by contacting the transformed cell with a non-endogenous ligand. In some aspects, the method further comprises culturing the transformed cell under conditions suitable for expression of the chimeric protein prior to inducing the chimeric protein to modulate transcription. In some aspects, modulating transcription comprises activating transcription of the gene of interest. In some aspects, the target expression cassette is encoded by the heterologous construct encoding a chimeric protein as described herein or the target expression cassette is encoded by a second heterologous construct. Exemplary non-endogenous ligands are provided herein. In some aspects, the non-endogenous ligand is selected from: 4-hydroxytamoxifen, N-desmethyltamoxifen, tamoxifen-N-oxide, and endoxifen. In some aspects, the gene of interest is selected from: a caspase domain (e.g., any one of caspases 1-11) or derivative or functional fragment thereof, optionally wherein the caspase is selected from caspase 3, caspase 6, caspase 7, caspase 8, caspase 9, or derivatives or functional fragments thereof, respectively, Diphtheria toxin fragment A (DTA), Bax, Bak, Bok, Bad, Bcl-Xs, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B1, or Purine nucleoside phosphorylase.

Also provided for herein is a method of modulating localization of a chimeric protein comprising transforming a cell with a heterologous construct encoding a chimeric protein as described herein, and inducing nuclear localization of the chimeric protein by contacting the transformed cell with a non-endogenous ligand. In some aspects, the method further comprising culturing the transformed cell under conditions suitable for expression of the chimeric protein prior to inducing the nuclear localization. Exemplary non-endogenous ligands are provided herein. In some aspects, the non-endogenous ligand is selected from: 4-hydroxytamoxifen, N-desmethyltamoxifen, tamoxifen-N-oxide, and endoxifen.

In some aspects, the transformed cell is in a human or animal, and wherein contacting the transformed cell with the non-endogenous ligand comprises administering a pharmacological dose of the ligand to the human or animal.

In some aspects, the non-endogenous ligand is administered at a concentration at which the non-endogenous ligand is substantially inactive on a wild-type estrogen receptor alpha of SEQ ID NO: 1.

Provided herein are modified ER-LBD comprising an amino acid sequence corresponding to a hormone binding domain of a reference human estrogen receptor sequence (SEQ ID NO: 1) and one or more additional amino acid substitutions, wherein the one or more additional amino acid substitutions are with reference to one or more regions selected from: positions 343-354, positions 380-392, positions 404-463, and positions 517-540, and position 547 of SEQ ID NO: 1. In some aspects, the modified ER-LBD as described herein further comprises a G400V amino acid substitution, an M543A amino acid substitution, and an L544A amino acid substitution, with reference to SEQ ID NO: 1. In some aspects, the modified ER-LBD further comprises a G400V amino acid substitution, an M543A amino acid substitution, an L544A, and a V595A amino acid substitution, with reference to SEQ ID NO: 1.

In some aspects, the modified ER-LBD comprises a G400V amino acid substitution, an M543A amino acid substitution, and an L544A amino acid substitution, and one or more additional amino acid substitutions. In some aspects, the modified ER-LBD has greater sensitivity to a non-endogenous ligand as compared to an ER-LBD comprising the amino acid sequence of SEQ ID NO: 2. In some aspects, the modified ER-LBD has greater selectivity to a non-endogenous ligand as compared to an ER-LBD comprising the amino acid sequence of SEQ ID NO: 2.

In some aspects, the modified ER-LBD comprises a G400V amino acid substitution, an M543A amino acid substitution, an L544A amino acid substitution, and a V595A amino acid substitution, and one or more additional amino acid substitutions. In some aspects, the modified ER-LBD has greater sensitivity to a non-endogenous ligand as compared to an ER-LBD comprising the amino acid sequence of SEQ ID NO: 3. In some aspects, the modified ER-LBD has greater selectivity to a non-endogenous ligand as compared to an ER-LBD comprising the amino acid sequence of SEQ ID NO: 3.

In some aspects, a modified ER-LBD of the present disclosure has greater sensitivity to a non-endogenous ligand as compared to an endogenous ligand as a result of the one or more additional amino acid substitutions.

In some aspects, a modified ER-LBD of the present disclosure has greater sensitivity to a non-endogenous ligand as compared to an ER-LBD comprising the amino acid sequence of SEQ ID NO: 2 or SEQ ID NO: 3.

In some aspects, a modified ER-LBD of the present disclosure has greater selectivity to a non-endogenous ligand as compared to an ER-LBD comprising the amino acid sequence of SEQ ID NO: 2 or SEQ ID NO: 3.

In some aspects, the one or more additional amino acid substitutions are at one or more positions of SEQ ID NO: 1 selected from: 343, 344, 345, 346, 347, 348, 349, 350, 351, 352, 354, 380, 384, 386, 387, 388, 389, 391, 392, 404, 407, 409, 413, 414, 417, 418, 420, 421, 422, 424, 428, 463, 517, 521, 522, 524, 525, 526, 527, 528, 533, 534, 536, 537, 538, 539, 540, and 547.

In some aspects, the one or more positions include position 343 of SEQ ID NO: 1. In some aspects the amino acid substitution at position 343 is selected from the group consisting of: M343F, M343I, M343L, and M343V.

In some aspects, the one or more positions include position 344 of SEQ ID NO: 1. In some aspects the amino acid substitution at position 344 is G344M.

In some aspects, the one or more positions include position 345 of SEQ ID NO: 1. In some aspects the amino acid substitution at position 345 is L345S.

In some aspects, the one or more positions include position 346 of SEQ ID NO: 1. In some aspects the amino acid substitution at position 346 is selected from the group consisting of: L346I, L346M, L346F, and L346V.

In some aspects, the one or more positions include position 347 of SEQ ID NO: 1. In some aspects the amino acid substitution at position 347 is selected from the group consisting of: T347D, T347E, T347F, T347I, T347K, T347L, T347M, T347N, T347Q, T347R, T347S, and T347V.

In some aspects, the one or more positions include position 348 of SEQ ID NO: 1. In some aspects the amino acid substitution at position 348 is N348K.

In some aspects, the one or more positions include position 349 of SEQ ID NO: 1. In some aspects the amino acid substitution at position 349 is selected from the group consisting of: L349I, L349M, L349F, and L349V.

In some aspects, the one or more positions include position 350 of SEQ ID NO: 1. In some aspects the amino acid substitution at position 350 is selected from the group consisting of: A350F, A350I, A350L, A350M and A350V.

In some aspects, the one or more positions include position 351 of SEQ ID NO: 1. In some aspects the amino acid substitution at position 351 is selected from the group consisting of: D351E, D351F, D351I, D351L, D351M, D351N, D351Q, and D351V.

In some aspects, the one or more positions include position 352 of SEQ ID NO: 1. In some aspects the amino acid substitution at position 352 is R352K.

In some aspects, the one or more positions include position 354 of SEQ ID NO: 1. In some aspects the amino acid substitution at position 354 is selected from the group consisting of: L354I, L354M, L354F, and L354V.

In some aspects, the one or more positions include position 380 of SEQ ID NO: 1. In some aspects the amino acid substitution at position 380 is E380Q.

In some aspects, the one or more positions include position 384 of SEQ ID NO: 1. In some aspects the amino acid substitution at position 384 is selected from the group consisting of: L384I, L384M, L384F, and L384V.

In some aspects, the one or more positions include position 386 of SEQ ID NO: 1. In some aspects the amino acid substitution at position 386 is I386V.

In some aspects, the one or more positions include position 387 of SEQ ID NO: 1. In some aspects the amino acid substitution at position 387 is selected from the group consisting of: L387I, L387M, L387F, and L387V.

In some aspects, the one or more positions include position 388 of SEQ ID NO: 1. In some aspects the amino acid substitution at position 388 is selected from the group consisting of: M388I, M388L, and M388F.

In some aspects, the one or more positions include position 389 of SEQ ID NO:

1. In some aspects the amino acid substitution at position 389 is I389M.

In some aspects, the one or more positions include position 391 of SEQ ID NO:

1. In some aspects the amino acid substitution at position 391 is selected from the group consisting of: L391I, L391M, L391F, and L391V.

In some aspects, the one or more positions include position 392 of SEQ ID NO:

1. In some aspects the amino acid substitution at position 392 is V392M.

In some aspects, the one or more positions include position 404 of SEQ ID NO:

1. In some aspects the amino acid substitution at position 404 is selected from the group consisting of: F404I, F404L, F404M, and F404V.

In some aspects, the one or more positions include position 407 of SEQ ID NO: 1. In some aspects the amino acid substitution at position 407 is N407D.

In some aspects, the one or more positions include position 409 of SEQ ID NO: 1. In some aspects the amino acid substitution at position 409 is LA09V.

In some aspects, the one or more positions include position 413 of SEQ ID NO: 1. In some aspects the amino acid substitution at position 413 is N413D.

In some aspects, the one or more positions include position 414 of SEQ ID NO: 1. In some aspects the amino acid substitution at position 414 is Q414E.

In some aspects, the one or more positions include position 417 of SEQ ID NO: 1. In some aspects the amino acid substitution at position 417 is C417S.

In some aspects, the one or more positions include position 418 of SEQ ID NO: 1. In some aspects the amino acid substitution at position 418 is selected from the group consisting of: V418I, V418L, V418M, and V418F.

In some aspects, the one or more positions include position 420 of SEQ ID NO: 1. In some aspects the amino acid substitution at position 420 is selected from the group consisting of: G420I, G420M, G420F, and G420V.

In some aspects, the one or more positions include position 421 of SEQ ID NO: 1. In some aspects the amino acid substitution at position 421 is selected from the group consisting of: M421I, M421L, M421F, and M421V.

In some aspects, the one or more positions include position 422 of SEQ ID NO: 1. In some aspects the amino acid substitution at position 422 is V422I.

In some aspects, the one or more positions include position 424 of SEQ ID NO: 1. In some aspects the amino acid substitution at position 424 is selected from the group consisting of: 1424L, 1424M, I424F, and I424V.

In some aspects, the one or more positions include position 428 of SEQ ID NO: 1. In some aspects the amino acid substitution at position 428 is selected from the group consisting of: L428I, LA28M, L428F, and L428V.

In some aspects, the one or more positions include position 463 of SEQ ID NO: 1. In some aspects the amino acid substitution at position 463 is S463P.

In some aspects, the one or more positions include position 517 of SEQ ID NO: 1. In some aspects the amino acid substitution at position 517 is M517A.

In some aspects, the one or more positions include position 521 of SEQ ID NO: 1. In some aspects the amino acid substitution at position 521 is selected from the group consisting of: G521A, G521F, G521I, G521L, G521M, and G521V.

In some aspects, the one or more positions include position 522 of SEQ ID NO: 1. In some aspects the amino acid substitution at position 522 is selected from the group consisting of: M522I, M522L, and M522V.

In some aspects, the one or more positions include position 524 of SEQ ID NO: 1. In some aspects the amino acid substitution at position 524 is selected from the group consisting of: H524A, H524I, H524L, H524F, and H524V.

In some aspects, the one or more positions include position 525 of SEQ ID NO: 1. In some aspects the amino acid substitution at position 525 is selected from the group consisting of: L525F, L525I, L525M, L525N, L525Q, L525S, L525T, and L525V.

In some aspects, the one or more positions include position 526 of SEQ ID NO: 1. In some aspects the amino acid substitution at position 526 is Y526L.

In some aspects, the one or more positions include position 527 of SEQ ID NO: 1. In some aspects the amino acid substitution at position 527 is S527N.

In some aspects, the one or more positions include position 528 of SEQ ID NO: 1. In some aspects the amino acid substitution at position 528 is selected from the group consisting of: M528F, M528I, and M528V.

In some aspects, the one or more positions include position 533 of SEQ ID NO: 1. In some aspects the amino acid substitution at position 533 is selected from the group consisting of: V533F and V533W.

In some aspects, the one or more positions include position 534 of SEQ ID NO: 1. In some aspects the amino acid substitution at position 534 is selected from the group consisting of: V534Q and V534R.

In some aspects, the one or more positions include position 536 of SEQ ID NO: 1. In some aspects the amino acid substitution at position 536 is selected from the group consisting of: L536F, and L536M, L536R, and L536Y.

In some aspects, the one or more positions include position 537 of SEQ ID NO: 1. In some aspects the amino acid substitution at position 537 is selected from the group consisting of: Y537E and Y537S.

In some aspects, the one or more positions include position 538 of SEQ ID NO: 1. In some aspects the amino acid substitution at position 538 is selected from the group consisting of: D538G and D538K.

In some aspects, the one or more positions include position 539 of SEQ ID NO: 1. In some aspects the amino acid substitution at position 539 is selected from the group consisting of: L539A and L539R.

In some aspects, the one or more positions include position 540 of SEQ ID NO: 1. In some aspects the amino acid substitution at position 540 is selected from the group consisting of: L540A and L540F.

In some aspects, the one or more positions include position 547 of SEQ ID NO: 1. In some aspects the amino acid substitution at position 547 is H547A.

In some aspects, the one or more additional amino acid substitutions include two amino acid substitutions. In some aspects, each of the two amino acid substitutions are at a position of SEQ ID NO: 1 selected from: 343, 345, 347, 348, 351, 354, 384, 387, 388, 389, 391, 392, 404, 418, 421, 521, 524, and 525.

In some aspects, the two amino acid substitutions are at positions 345 and 348 of SEQ ID NO: 1. In some aspects, the amino acid substitution at position 345 of SEQ ID NO: 1 is L345S and the amino acid substitution at position 348 of SEQ ID NO: 1 is N348K.

In some aspects, the two amino acid substitutions are at positions 384 and 389 of SEQ ID NO: 1. In some aspects, the amino acid substitution at position 384 of SEQ ID NO: 1 is L384M and the amino acid substitution at position 389 of SEQ ID NO: 1 is I389M.

In some aspects, the two amino acid substitutions are at positions 421 and 392 of SEQ ID NO: 1. In some aspects, the amino acid substitution at position 421 of SEQ ID NO: 1 is M421I and the amino acid substitution at position 392 of SEQ ID NO: 1 is V392M.

In some aspects, the two amino acid substitutions are at positions 354 and 391 of SEQ ID NO: 1. In some aspects, the amino acid substitution at position 354 of SEQ ID NO: 1 is L354I and the amino acid substitution at position 391 of SEQ ID NO: 1 is L391F.

In some aspects, the two amino acid substitutions are at positions 354 and 384 of SEQ ID NO: 1. In some aspects, the amino acid substitution at position 354 of SEQ ID NO: 1 is L354I and the amino acid substitution at position 384 of SEQ ID NO: 1 is L384M.

In some aspects, the two amino acid substitutions are at positions 354 and 387 of SEQ ID NO: 1. In some aspects, the amino acid substitution at position 354 of SEQ ID NO: 1 is L354I and the amino acid substitution at position 387 of SEQ ID NO: 1 is L387M.

In some aspects, the two amino acid substitutions are at positions 387 and 391. In some aspects, the amino acid substitution at position 387 of SEQ ID NO: 1 is L387M and the amino acid substitution at position 391 of SEQ ID NO: 1 is L391F.

In some aspects, the two amino acid substitutions are at positions 384 and 387 of SEQ ID NO: 1. In some aspects, the amino acid substitution at position 384 of SEQ ID NO: 1 is L384M and the amino acid substitution at position 387 of SEQ ID NO: 1 is L387M.

In some aspects, the two amino acid substitutions are at positions 384 and 391 of SEQ ID NO: 1. In some aspects, the amino acid substitution at position 384 of SEQ ID NO: 1 is L384M and the amino acid substitution at position 391 of SEQ ID NO: 1 is L391F.

In some aspects, the one or more additional amino acid substitutions include three amino acid substitutions. In some aspects, the three amino acid substitutions are each at a position of SEQ ID NO: 1 selected from the group consisting of: 343, 347, 351, 354, 388, 391, 404, 418, 521, 524, and 525.

In some aspects, the three amino acid substitutions are at positions 354, 384, and 391 of SEQ ID NO: 1. In some aspects, the amino acid substitution at position 354 of SEQ ID NO: 1 is L354I, the amino acid substitution at position 384 of SEQ ID NO: 1 is L384M, and the amino acid substitution at position 391 of SEQ ID NO: 1 is L391F.

In some aspects, the one or more additional amino acid substitutions include four amino acid substitutions.

In some aspects, the four amino acid substitutions are at positions 354, 384, 391, and 418 of SEQ ID NO: 1. In some aspects, the amino acid substitution at position 354 of SEQ ID NO: 1 is L354I, the amino acid substitution at position 384 of SEQ ID NO: 1 is L384M, the amino acid substitution at position 391 of SEQ ID NO: 1 is L391F, and the amino acid substitution at position 418 of SEQ ID NO: 1 is V418I.

In some aspects, the four amino acid substitutions are at positions 343, 388, 521, and 404 of SEQ ID NO: 1. In some aspects, the amino acid substitution at position 343 of SEQ ID NO: 1 is M343I, the amino acid substitution at position 388 of SEQ ID NO: 1 is M388I, the amino acid substitution at position 521 of SEQ ID NO: 1 is G521I, and the amino acid substitution at position 404 of SEQ ID NO: 1 is F404L.

In some aspects, the four amino acid substitutions are at positions 524, 347, 351, and 525 of SEQ ID NO: 1. In some aspects, the amino acid substitution at position 524 of SEQ ID NO: 1 is H524V, the amino acid substitution at position 347 of SEQ ID NO: 1 is T347R, the amino acid substitution at position 351 of SEQ ID NO: 1 is D351Q, and the amino acid substitution at position 525 of SEQ ID NO: 1 is L525N.

Exemplary non-endogenous ligands are provided herein. In some aspects, the non-endogenous ligand is selected from: 4-hydroxytamoxifen (4-OHT), N-desmethyltamoxifen, tamoxifen-N-oxide, and endoxifen.

Also provided are chimeric proteins including a polypeptide of interest fused to a modified ER-LBD as described herein. In some aspects, the polypeptide of interest includes a nucleic acid binding domain. In some aspects, the nucleic acid binding domain includes a zinc finger (ZF) domain. In some aspects, the chimeric protein is a transcription factor and the polypeptide of interest includes a transcriptional modulator domain.

Also provided are isolated polynucleotides encoding modified ER-LBD as described herein or the chimeric protein as described herein.

Also provided are heterologous constructs including a promoter operably linked to a polynucleotide encoding a modified ER-LBD as described herein or a chimeric protein as described herein.

Also provided are plasmids and vectors comprising the heterologous constructs as described herein.

Also provided are cells (such as an isolated cell or a population of cells) including a heterologous construct as described herein or a plasmid as described herein.

Also provided is a molecular switch for modulating transcription of a gene of interest. In some aspects, the molecular switch includes a chimeric protein including a modified ER-LBD as described herein and a transcription modulator, and a non-endogenous ligand, wherein binding of the non-endogenous ligand to the modified ER-LBD induces the chimeric protein to modulate transcription of the gene of interest. Exemplary non-endogenous ligands are provided herein. In some aspects, the non-endogenous ligand of the molecular switch is selected from: 4-hydroxytamoxifen (4-OHT), N-desmethyltamoxifen, tamoxifen-N-oxide, and endoxifen.

Also provided herein is a method of modulating transcription of a gene of interest. In some aspects, the method includes (a) transforming a cell with (i) a heterologous construct encoding the chimeric protein including a modified ER-LBD and a transcriptional modulator domain, and (ii) a target expression cassette comprising a gene of interest; (b) culturing the transformed call under conditions suitable for expression of the chimeric protein; and (c) inducing the chimeric protein to modulate transcription of the gene of interest by contacting the transformed cell with a non-endogenous ligand.

In some aspects, the method of modulating transcription is a method of activating transcription.

In some aspects, the method of modulating transcription is a method of repressing transcription.

In some aspects, the target expression cassette is encoded by the heterologous construct encoding the chimeric protein.

In some aspects, the target expression cassette is encoded by a different heterologous construct from the heterologous construct encoding the chimeric protein.

Also provided is a method of modulating localization of a polypeptide of interest. In some aspects, the method includes (a) transforming a cell with a heterologous construct encoding a chimeric protein including a polypeptide of interest fused to a modified ER-LBD as described herein; (b) culturing the transformed cell under conditions suitable for expression of the chimeric protein; and (c) inducing nuclear localization of the chimeric protein by contacting the transformed cell with a non-endogenous ligand.

In some aspects, the transformed cell of any of the methods described herein is in a human or an animal. In some aspects, contacting the transformed cell with the non-endogenous ligand comprises administering a pharmacological dose of the ligand to the human or animal.

Exemplary non-endogenous ligands are provided herein. In some aspects, the non-endogenous ligand of step (c) of the previously described methods is selected from: 4-hydroxytamoxifen, N-desmethyltamoxifen, tamoxifen-N-oxide, and endoxifen.

In some embodiments, the non-endogenous ligand is administered at a concentration at which the non-endogenous ligand is substantially inactive on wild-type estrogen receptor alpha.

### BRIEF DESCRIPTION OF THE SEVERAL VIEWS OF THE DRAWINGS

These and other features, aspects, and advantages of the present disclosure will become better understood with regard to the following description, and accompanying drawings.

FIG. 1A and FIG. 1B, provide binding energy calculations for the first set of mutations analyzed in silico. FIG. 1A provides binding energy calculations for binding to estradiol, FIG. 1B provides binding energy calculations for binding to 4-OHT.

FIG. 2 provides binding energy calculations for 4-OHT binding, for the second set of mutations analyzed in silico.

FIG. 3 provides binding energy calculations for 4-OHT binding, for the third set of mutations analyzed in silico.

FIG. 4 provides binding energy calculations for 4-OHT binding, for the fourth set of mutations analyzed in silico.

FIG. 5 provides binding energy calculations for 4-OHT binding, for the fifth set of mutations analyzed in silico.

FIG. 6 shows structural differences between the estradiol-bound and non-endogenous ligand-bound conformations in the orientation and docking site of helix 12.

FIG. 7 provides binding energy calculations for the agonist-bound versus the antagonist-bound conformation, for the sixth set of mutations analyzed in silico.

FIG. 8A, FIG. 8B, and FIG. 8C show the effect of various modified ER-LBDs on reporter expression over various concentrations of 4-OHT, as assayed in a first transfection screen.

FIG. 9A, FIG. 9B, and FIG. 9C show the effect of various modified ER-LBDs on reporter expression over various concentrations of 4-OHT, as assayed in a second transfection screen.

FIG. 10A, FIG. 10B, and FIG. 10C show the effect of various modified ER-LBDs on reporter expression over various concentrations of 4-OHT, as assayed in a third transfection screen.

FIG. 11A and FIG. 11B show the effect of various modified ER-LBDs on reporter expression over various concentrations of 4-OHT, as assayed in a first transduction screen.

FIG. 12 shows the effect of various modified ER-LBDs on reporter expression over various concentrations of 4-OHT, as assayed in a second transduction screen.

FIG. 13 shows the effect of various modified ER-LBDs on reporter expression over various concentrations of 4-OHT, as assayed in a second transduction screen.

FIG. 14A and FIG. 14B show a backbone for high-throughput protein engineering of ERT2 (SB04401) and an OFF mCherry reporter construct (SB01066).

FIG. 15A and FIG. 15B show the effect of various modified ER-LBDs on reporter expression over various concentrations of endoxifen and 4-OHT, as assayed in a combinatorial library screen.

FIG. 16A, FIG. 16B, FIG. 16C, FIG. 16D, and FIG. 16E show the effect of various modified ER-LBDs on reporter expression over various concentrations of endoxifen, 4-OHT, and estradiol, as assayed in a validation screen. SB03422 is the wild-type ER-LBD and is included as a benchmark for performance.

FIG. 17A and FIG. 17B shows the effect of various modified ER-LBDs on reporter expression over various concentrations of endoxifen and 4-OHT, as assayed in NK cells. Arrow indicates estimated pharmacologically relevant 4-OHT or endoxifen concentrations in humans.

FIG. 18A and FIG. 18B show the effect of various modified ER-LBDs on IL-12 expression over various concentrations of endoxifen, as assayed in NK cells.

FIG. 19 illustrates exemplary mechanisms of action for inducible cell-death systems, including ER-mediated transcriptional induction via nuclear localization (top panel) and ER-mediated suicide-switch killing via dimerization (bottom panel).

FIG. 20 shows suicide-switch induced killing for constructs with modified ER-LBD variants in HEK293T cells at 1 μM 4-OHT over a period of 48 hours.

FIG. 21 shows suicide-switch induced killing for a construct with a modified ER-LBD variant in HEK293T cells over a period of 48 hours at the indicated concentrations of 4-OHT.

FIG. 22 shows suicide-switch induced killing for a construct with a modified ER-LBD variant in transduced primary T cells at 5 days at the indicated treatment conditions.

FIG. 23 shows suicide-switch induced killing for a construct with a modified ER-LBD variant in transduced primary T cells over the indicated period of time and at the indicated treatment conditions.

FIG. 24 shows results of a time course experiment for indicated drug conditions, assessing exemplary ERT2 mutant safety switch activity in HEK cells.

FIG. 25 shows results of a time course experiment for indicated drug conditions, assessing exemplary ERT2 mutant safety switch activity in HEK cells.

FIG. 26A depicts constructs and experimental methods for evaluating activity of exemplary ERT2 mutant transcriptional switches. FIG. 26B shows log 10 fold activation across a range of endoxifen concentrations, normalized to no virus control.

FIG. 26C shows induced mCherry expression plotted against basal activity for each of the tested ERT2 transcriptional switch constructs.

FIG. 27 shows results of an experiment evaluating activity of exemplary ERT2 mutant transcriptional switches, where the readout is mCherry gMFI after background subtraction across a range of estradiol and endoxifen concentrations.

FIG. 28A depicts constructs and experimental methods for evaluating exemplary ERT2 mutant transcriptional switches for inducing expression of an IL-12 payload. FIGS. 28B and 28C show results of an experiment evaluating exemplary ERT2 mutant transcriptional switches for inducing expression of an IL-12 payload.

FIG. 29A shows an experimental workflow for evaluating suicide switch activity for a number of additional ERT2 mutant safety switch constructs. FIG. 29B shows results of an experiment evaluating suicide switch activity of the additional ERT2 mutant safety switch constructs.

### DETAILED DESCRIPTION OF THE INVENTION

Terms used in the claims and specification are defined as set forth below unless otherwise specified.

The term “in vivo” refers to processes that occur in a living organism.

The term “mammal” as used herein includes both humans and non-humans and include but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can be a “prophylactically effective amount” as prophylaxis can be considered therapy.

It must be noted that, as used in the specification and the appended claims, the singular forms “a,” “an” and “the” include plural referents unless the context clearly dictates otherwise.

“The term “about” is used herein to provide literal support for the exact term that it precedes, and allows for near approximations of the term. For example, certain ranges or similarity may be presented with numerical ranges preceded by the term “about”. If the degree of approximation is not otherwise clear from the context, “about” means either within plus or minus 10% of the provided value, or rounded to the nearest significant figure, in all cases inclusive of the provided value. In some embodiments, the term “about” indicates the designated value±up to 10%, up to +5%, or up to +1%.

### Inducible Cell Death Systems

The ability to induce cell death using synthetic ligands, particularly those approved for clinical use, would be beneficial in therapeutic applications, such as in the field of cell and gene therapy. Cell and gene therapies show great clinical promise; however, they carry a risk of off-target toxicity that can result in organ damage and patient deaths. It can be desirable to engineer cell and gene therapy products with a “safety” switch, e.g., an inducible cell death system, to address potential toxicity concerns.

Synthetic ligands that bind to ER have been developed for treating ER-positive cancers such as ER-positive breast cancer. For example, active metabolites of the drug tamoxifen induce nuclear translocation of ER and antagonize ER in a tissue-selective manner.

Tamoxifen and its active metabolites are also utilized as a tool for controlling nuclear localization in the research setting. For example, an ER ligand binding domain variant known as ERT2 has been used as a fusion protein with Cre recombinase to regulate Cre recombinase-based gene editing in animal model systems.

As shown herein, ERT2 mutants engineered with additional amino acid substitutions can be implemented into inducible cell death systems to induce potent cell killing. Accordingly, provided herein are inducible cell death systems that include modified estrogen receptor ligand binding domains (ER-LBD). The inducible cell death systems described herein can include a polypeptide, wherein the polypeptide includes a ligand binding domain and a cell death inducing domain. In some embodiments, a polypeptide herein is configured, upon contact with a ligand of the ligand binding domain, to regulate a cell-death inducing pathway in a cell in which the polypeptide is expressed. In some embodiments, a polypeptide herein is configured, upon contact with a ligand of the ligand binding domain, to generate a cell-death inducing signal in a cell in which the polypeptide is expressed.

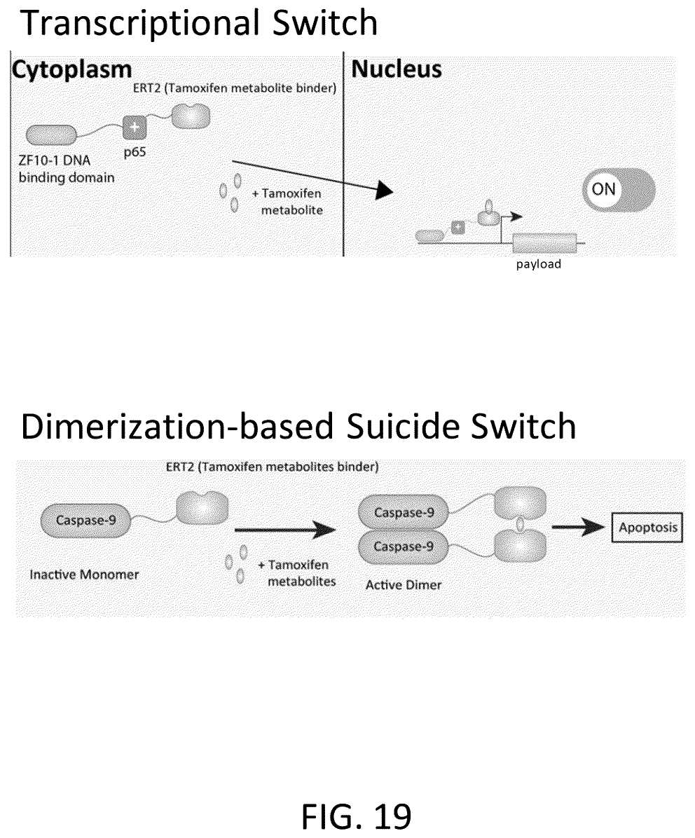

FIG. 19 shows exemplary inducible cell-death systems, including ER-mediated transcriptional induction via nuclear localization (top panel) and ER-mediated suicide-switch killing via dimerization (bottom panel).

In some embodiments, a polypeptide herein is configured, upon contact with a ligand of the ligand binding domain, to regulate expression of polynucleotides and/or further polypeptides of a cell-death inducing pathway in a cell in which the polypeptide is expressed. In exemplary embodiments of a polypeptide comprising a modified ER-LBD as disclosed herein and a cell death inducing domain, the cell death-inducing domain is a transcription factor comprising a nucleic acid-binding domain disclosed herein (e.g., ZF10-1 DNA binding domain as depicted in FIG. 19, top panel) and a transcriptional effector domain, also referred to herein as a transcription modulator (e.g., p65 as depicted in FIG. 19, top panel). Without wishing to be bound by theory, upon contact of the ligand with the ligand binding domain (e.g., wherein the contacting comprises binding of the ligand to the ligand binding domain), the polypeptide may translocate to the nucleus to activate expression of a death inducing payload.

The inducible cell death system includes systems where a polypeptide is or comprises a first polypeptide monomer and the inducible cell-death system further includes a second polypeptide monomer. Polypeptide monomers” refer to proteins, protein subunits, and/or protein domains that are configured to, upon contact with a ligand of the ER-LBD (e.g., wherein the contacting comprises binding of the ligand to the ligand binding domains of the first and the second polypeptide monomers), generate a cell-death inducing signal in a cell in which the first and the second polypeptide monomers are expressed. In some embodiments, the first polypeptide monomer and the second polypeptide monomer are configured to oligomerize with each other upon contact with a ligand of the ligand binding domain (e.g., wherein the contacting comprises binding of the ligand to the ligand binding domains of the first and the second polypeptide monomers), thereby generating the cell-death inducing signal in a cell in which the first polypeptide monomer and the second polypeptide monomer are expressed. Without wishing to be bound by theory, binding of the ligand binding domains of the first and the second polypeptide monomers with the ligand may induce oligomerization of the polypeptide monomers, wherein the oligomerization generates the cell-death inducing signal by activating the cell death inducing domain. See, e.g., FIG. 19 (bottom panel, depicting caspase 9 as an exemplary cell death inducing domain).

Such inducible cell death systems can beneficially improve the safety profile of cell and gene therapy products.

Exemplary cell death-inducing domains and/or death-inducing payloads can be derived from a protein such as one or more of: a caspase (e.g., any one of caspase 1-11, such as caspase 3, caspase 6, caspase 7, caspase 8, caspase 9), Diphtheria toxin fragment A (DTA), Bax, Bak, Bok, Bad, Bcl-Xs, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related apoptosis-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B1, and/or Purine nucleoside phosphorylase. Exemplary sequences can be found in Table A.

TABLE A

| | SEQ | | SEQ | |
|---|---|---|---|---|
| Component | ID | | ID | |
| Name | NO | Amino acid sequence | NO | Nucleic acid sequence |
| 4x ZF10-1 | | NA | 59 | CGGGTTTCGTAACAATCGCATGAGGATTCG |
| binding site | | | | CAACGCCTTCGGCGTAGCCGATGTCGCGCTC |
| (BS) | | | | CCGTCTCAGTAAAGGTCGGCGTAGCCGATG |
| | | | | TCGCGCAATCGGACTGCCTTCGTACGGCGTA |
| | | | | GCCGATGTCGCGCGTATCAGTCGCCTCGGA |
| | | | | ACGGCGTAGCCGATGTCGCGCATTCGTAAG |
| | | | | AGGCTCACTCTCCCTTACACGGAGTGGATA |
| Human Bax | 32 | DGSGEQPRGGGPTSSEQIMKTGALLLQGFI | 60 | GACGGGTCCGGGGAGCAGCCCAGAGGCGG |
| | | QDRAGRMGGEAPELALDPVPQDASTKKL | | GGGGCCCACCAGCTCTGAGCAGATCATGAA |
| | | SECLKRIGDELDSNMELQRMIAAVDTDSP | | GACAGGGGCCCTTTTGCTTCAGGGTTTCATC |
| | | REVFFRVAADMFSDGNFNWGRVVALFYF | | CAGGATCGAGCAGGGCGAATGGGTGGAGA |
| | | ASKLVLKALCTKVPELIRTIMGWTLDFLR | | GGCACCCGAGCTGGCCCTGGACCCGGTGCC |
| | | ERLLGWIQDQGGWDGLLSYFGTPTWQTV | | TCAGGATGCGTCCACCAAGAAGCTGAGCGA |
| | | TIFVAGVLTASLTIWKKMG | | GTGTCTCAAGCGCATCGGGGACGAACTGGA |
| | | | | CAGTAACATGGAGCTGCAGAGGATGATTGC |
| | | | | CGCCGTGGACACAGACTCCCCCCGAGAGGT |
| | | | | CTTTTTCCGAGTGGCAGCTGACATGTTTTCT |
| | | | | GACGGCAACTTCAACTGGGGCCGGGTTGTC |
| | | | | GCCCTTTTCTACTTTGCCAGCAAACTGGTGC |
| | | | | TCAAGGCCCTGTGCACCAAGGTGCCGGAAC |
| | | | | TGATCAGAACCATCATGGGCTGGACATTGG |
| | | | | ACTTCCTCCGGGAGCGGCTGTTGGGCTGGAT |
| | | | | CCAAGACCAGGGTGGTTGGGACGGCCTCCT |
| | | | | CTCCTACTTTGGGACGCCCACGTGGCAGACC |
| | | | | GTGACCATCTTTGTGGCGGGAGTGCTCACCG |
| | | | | CCTCACTCACCATCTGGAAGAAGATGGGC |
| Caspase 9 | 39 | DEADRRLLRRCRLRLVEELQVDQLWDVL | 67 | GACGAAGCGGATCGGCGGCTCCTGCGGCGG |
| (full, non- | | LSRELFRPHMIEDIQRAGSGSRRDQARQLII | | TGCCGGCTGCGGCTGGTGGAAGAGCTGCAG |
| truncated | | DLETRGSQALPLFISCLEDTGQDMLASFLR | | GTGGACCAGCTCTGGGACGTCCTGCTGAGC |
| sequence) | | TNRQAAKLSKPTLENLTPVVLRPEIRKPEV | | CGCGAGCTGTTCAGGCCCCATATGATCGAG |
| | | LRPETPRPVDIGSGGFGDVGALESLRGNA | | GACATCCAGCGGGCAGGCTCTGGATCTCGG |
| | | DLAYILSMEPCGHCLIINNVNFCRESGLRT | | CGGGATCAGGCCAGGCAGCTGATCATAGAT |
| | | RTGSNIDCEKLRRRFSSLHFMVEVKGDLT | | CTGGAGACTCGAGGGAGTCAGGCTCTTCCTT |
| | | AKKMVLALLELARQDHGALDCCVVVILS | | TGTTCATCTCCTGCTTAGAGGACACAGGCCA |
| | | HGCQASHLQFPGAVYGTDGCPVSVEKIVN | | GGACATGCTGGCTTCGTTTCTGCGAACTAAC |
| | | IFNGTSCPSLGGKPKLFFIQACGGEQKDHG | | AGGCAAGCAGCAAAGTTGTCGAAGCCAACC |
| | | FEVASTSPEDESPGSNPEPDATPFQEGLRTF | | CTAGAAAACCTTACCCCAGTGGTGCTCAGA |
| | | DQLDAISSLPTPSDIFVSYSTFPGFVSWRDP | | CCAGAGATTCGCAAACCAGAGGTTCTCAGA |
| | | KSGSWYVETLDDIFEQWAHSEDLQSLLLR | | CCGGAAACACCCAGACCAGTGGACATTGGT |
| | | VANAVSVKGIYKQMPGCFNFLRKKLFFKT | | TCTGGAGGATTCGGTGATGTCGGTGCTCTTG |
| | | S | | AGAGTTTGAGGGGAAATGCAGATTTGGCTT |
| | | | | ACATCCTGAGCATGGAGCCCTGTGGCCACT |
| | | | | GCCTCATTATCAACAATGTGAACTTCTGCCG |
| | | | | TGAGTCCGGGCTCCGCACCCGCACTGGCTCC |
| | | | | AACATCGACTGTGAGAAGTTGCGGCGTCGC |
| | | | | TTCTCCTCGCTGCATTTCATGGTGGAGGTGA |
| | | | | AGGGCGACCTGACTGCCAAGAAAATGGTGC |
| | | | | TGGCTTTGCTGGAGCTGGCGCGGCAGGACC |
| | | | | ACGGTGCTCTGGACTGCTGCGTGGTGGTCAT |
| | | | | TCTCTCTCACGGCTGTCAGGCCAGCCACCTG |
| | | | | CAGTTCCCAGGGGCTGTCTACGGCACAGAT |
| | | | | GGATGCCCTGTGTCGGTCGAGAAGATTGTG |
| | | | | AACATCTTCAATGGGACCAGCTGCCCCAGC |
| | | | | CTGGGAGGGAAGCCCAAGCTCTTTTTCATCC |
| | | | | AGGCCTGTGGTGGGGAGCAGAAAGACCATG |
| | | | | GGTTTGAGGTGGCCTCCACTTCCCCTGAAGA |
| | | | | CGAGTCCCCTGGCAGTAACCCCGAGCCAGA |
| | | | | TGCCACCCCGTTCCAGGAAGGTTTGAGGAC |
| | | | | CTTCGACCAGCTGGACGCCATATCTAGTTTG |
| | | | | CCCACACCCAGTGACATCTTTGTGTCCTACT |
| | | | | CTACTTTCCCAGGTTTTGTTTCCTGGAGGGA |
| | | | | CCCCAAGAGTGGCTCCTGGTACGTTGAGAC |
| | | | | CCTGGACGACATCTTTGAGCAGTGGGCTCA |
| | | | | CTCTGAAGACCTGCAGTCCCTCCTGCTTAGG |
| | | | | GTCGCTAATGCTGTTTCGGTGAAAGGGATTT |
| | | | | ATAAACAGATGCCTGGTTGCTTTAATTTCCT |
| | | | | CCGGAAAAAACTTTTCTTTAAAACATCA |
| Diphtheria | 41 | DPDDVVDSSKSFVMENFSSYHGTKPGYV | 69 | GACCCTGATGATGTTGTTGATTCTTCTAAAT |
| toxin A | | DSIQKGIQKPKSGTQGNYDDDWKGFYSTD | | CTTTTGTGATGGAAAACTTTTCTTCGTACCA |
| (DTA) | | NKYDAAGYSVDNENPLSGKAGGVVKVT | | CGGGACTAAACCTGGTTATGTAGATTCCATT |
| | | YPGLTKVLALKVDNAETIKKELGLSLTEPL | | CAAAAAGGTATACAAAAGCCAAAATCTGGT |
| | | MEQVGTEEFIKRFGDGASRVVLSLPFAEG | | ACACAAGGAAATTATGACGATGATTGGAAA |
| | | SSSVEYINNWEQAKALSVELEINFETRGKR | | GGGTTTTATAGTACCGACAATAAATACGAC |
| | | GQDAMYEYMAQACAGNRVRRSLCEGTL | | GCTGCGGGATACTCTGTAGATAATGAAAAC |
| | | LLWCDIIGQTTYRDLKL | | CCGCTCTCTGGAAAAGCTGGAGGCGTGGTC |
| | | | | AAAGTGACGTATCCAGGACTGACGAAGGTT |
| | | | | CTCGCACTAAAAGTGGATAATGCCGAAACT |
| | | | | ATTAAGAAAGAGTTAGGTTTAAGTCTCACT |
| | | | | GAACCGTTGATGGAGCAAGTCGGAACGGAA |
| | | | | GAGTTTATCAAAAGGTTCGGTGATGGTGCTT |
| | | | | CGCGTGTAGTGCTCAGCCTTCCCTTCGCTGA |
| | | | | GGGGAGTTCTAGCGTTGAATATATTAATAA |
| | | | | CTGGGAACAGGCGAAAGCGTTAAGCGTAGA |
| | | | | ACTTGAGATTAATTTTGAAACCCGTGGAAA |
| | | | | ACGTGGCCAAGATGCGATGTATGAGTATAT |
| | | | | GGCTCAAGCCTGTGCAGGAAATCGTGTCAG |
| | | | | GCGATCTCTTTGTGAAGGAACCTTACTTCTG |
| | | | | TGGTGTGACATAATTGGACAAACTACCTAC |
| | | | | AGAGATTTAAAGCTC |
| ER | 42 | AGDMRAANLWPSPLMIKRSKKNSLALSLT | 70 | GCCGGCGATATGAGAGCTGCTAACCTGTGG |
| G400V/M543/ | | ADQMVSALLDAEPPILYSEYDPTRPFSEAS | | CCTTCTCCACTGATGATCAAGCGGTCCAAGA |
| L544A | | MMGLLTNLADRELVHMINWAKRVPGFV | | AGAACAGTCTGGCCCTGAGCCTGACCGCCG |
| mutations | | DLTLHDQVHLLECAWLEILMIGLVWRSM | | ACCAGATGGTTTCAGCACTGCTGGATGCCG |
| (Exemplary | | EHPVKLLFAPNLLLDRNQGKCVEGMVEIF | | AGCCTCCTATCCTGTACAGCGAGTACGACCC |
| ERT2) | | DMLLATSSRFRMMNLQGEEFVCLKSIILL | | CACCAGACCTTTTAGCGAGGCCAGCATGAT |
| | | NSGVYTFLSSTLKSLEEKDHIHRVLDKITD | | GGGCCTGCTGACCAATCTGGCCGACAGAGA |
| | | TLIHLMAKAGLTLQQQHQRLAQLLLILSHI | | ACTGGTGCACATGATCAACTGGGCCAAGCG |
| | | RHMSNKGMEHLYSMKCKNVVPLYDLLLE | | CGTGCCCGGCTTTGTGGATCTGACACTGCAC |
| | | AADAHRLHAPTSRGGASVEETDQSHLAT | | GACCAAGTGCATCTGCTCGAGTGCGCCTGG |
| | | AGSTSSHSLQKYYITGEAEGFPAT | | CTGGAAATCCTGATGATCGGACTCGTGTGG |
| | | | | CGGAGCATGGAACACCCTGTGAAGCTGCTG |
| | | | | TTCGCCCCTAACCTGCTGCTGGACAGAAACC |
| | | | | AGGGCAAATGCGTGGAAGGCATGGTGGAAA |
| | | | | TCTTCGATATGCTGCTGGCCACCTCCAGCCG |
| | | | | GTTCCGGATGATGAATCTGCAGGGCGAAGA |
| | | | | GTTCGTGTGCCTGAAGTCCATTATCCTGCTG |
| | | | | AACAGCGGCGTGTACACCTTTCTGAGCAGC |
| | | | | ACCCTGAAGTCTCTGGAAGAGAAGGACCAC |
| | | | | ATCCACAGAGTGCTGGACAAGATCACCGAC |
| | | | | ACACTGATCCACCTGATGGCCAAGGCCGGA |
| | | | | CTGACTCTGCAGCAGCAGCATCAAAGACTG |
| | | | | GCCCAGCTGCTGCTCATCCTGAGCCACATCA |
| | | | | GACACATGAGCAACAAAGGCATGGAACATC |
| | | | | TGTACAGCATGAAGTGCAAGAACGTGGTGC |
| | | | | CCCTGTACGATCTGCTGCTCGAAGCCGCTGA |
| | | | | TGCCCACAGACTGCATGCCCCTACATCTAGA |
| | | | | GGCGGAGCCTCCGTGGAAGAGACAGATCAG |
| | | | | TCTCATCTGGCCACCGCCGGCAGCACAAGC |
| | | | | TCTCATTCTCTGCAGAAGTACTACATCACCG |
| | | | | GCGAGGCCGAGGGATTTCCTGCCACA |
| GS linker 1 | 45 | GGGGSGGGGSGGGGSVDGF | 73 | GGGGGTGGAGGTTCAGGGGGTGGAGGTTCA |
| | | | | GGTGGTGGCGGTAGTGTCGATGGCTTC |
| GS linker 2 | 46 | ASGGGGSAS | 74 | GCTAGTGGCGGCGGCGGCAGTGCTAGT |
| TCR linker | 86 | TCR | 87 | ACCTGCAGG |
| QLCVRGSS | 88 | QLCVRGSS | 89 | CAATTGTGCGTACGCGGATCCTCT |
| linker | | | | |
| Granzyme B | 47 | QPILLLLAFLLLPRADAGEIIGGHEAKPHSR | 75 | CAACCAATCCTGCTTCTGCTGGCCTTCCTCC |
| | | PYMAYLMIWDQKSLKRCGGFLIQDDFVL | | TGCTGCCCAGGGCAGATGCAGGGGAGATCA |
| | | TAAHCWGSSINVTLGAHNIKEQEPTQQFIP | | TCGGGGGACATGAGGCCAAGCCCCACTCCC |
| | | VKRPIPHPAYNPKNFSNDIMLLQLERKAK | | GCCCCTACATGGCTTATCTTATGATCTGGGA |
| | | RTRAVQPLRLPSNKAQVKPGQTCSVAGW | | TCAGAAGTCTCTGAAGAGGTGCGGTGGCTT |
| | | GQTAPLGKHSHTLQEVKMTVQEDRKCES | | CCTGATACAAGACGACTTCGTGCTGACAGC |
| | | DLRHYYDSTIELCVGDPEIKKTSFKGDSGG | | TGCTCACTGTTGGGGAAGCTCCATAAATGTC |
| | | PLVCNKVAQGIVSYGRNNGMPPRACTKV | | ACCTTGGGGGCCCACAATATCAAAGAACAG |
| | | SSFVHWIKKTMKRH | | GAGCCGACCCAGCAGTTTATCCCTGTGAAA |
| | | | | AGACCCATCCCCCATCCAGCCTATAATCCTA |
| | | | | AGAACTTCTCCAACGACATCATGCTACTGCA |
| | | | | GCTGGAGAGAAAGGCCAAGCGGACCAGAG |
| | | | | CTGTGCAGCCCCTCAGGCTACCTAGCAACA |
| | | | | AGGCCCAGGTGAAGCCAGGGCAGACATGCA |
| | | | | GTGTGGCCGGCTGGGGGCAGACGGCCCCCC |
| | | | | TGGGAAAACACTCACACACACTACAAGAGG |
| | | | | TGAAGATGACAGTGCAGGAAGATCGAAAGT |
| | | | | GCGAATCTGACTTACGCCATTATTACGACAG |
| | | | | TACCATTGAGTTGTGCGTGGGGGACCCAGA |
| | | | | GATTAAAAAGACTTCCTTTAAGGGGGACTC |
| | | | | TGGAGGCCCTCTTGTGTGTAACAAGGTGGC |
| | | | | CCAGGGCATTGTCTCCTATGGACGAAACAA |
| | | | | TGGCATGCCTCCACGAGCCTGCACCAAAGT |
| | | | | CTCAAGCTTTGTACACTGGATAAAGAAAAC |
| | | | | CATGAAACGCCAC |
| iCasp9 | 48 | DVGALESLRGNADLAYILSMEPCGHCLIIN | 76 | GATGTCGGTGCTCTTGAGAGTTTGAGGGGA |
| | | NVNFCRESGLRTRTGSNIDCEKLRRRFSSL | | AATGCAGATTTGGCTTACATCCTGAGCATGG |
| | | HFMVEVKGDLTAKKMVLALLELARQDH | | AGCCCTGTGGCCACTGCCTCATTATCAACAA |
| | | GALDCCVVVILSHGCQASHLQFPGAVYGT | | TGTGAACTTCTGCCGTGAGTCCGGGCTCCGC |
| | | DGCPVSVEKIVNIFNGTSCPSLGGKPKLFFI | | ACCCGCACTGGCTCCAACATCGACTGTGAG |
| | | QACGGEQKDHGFEVASTSPEDESPGSNPE | | AAGTTGCGGCGTCGCTTCTCCTCGCTGCATT |
| | | PDATPFQEGLRTFDQLDAISSLPTPSDIFVS | | TCATGGTGGAGGTGAAGGGCGACCTGACTG |
| | | YSTFPGFVSWRDPKSGSWYVETLDDIFEQ | | CCAAGAAAATGGTGCTGGCTTTGCTGGAGC |
| | | WAHSEDLQSLLLRVANAVSVKGIYKQMP | | TGGCGCGGCAGGACCACGGTGCTCTGGACT |
| | | GCFNFLRKKLFFKTS | | GCTGCGTGGTGGTCATTCTCTCTCACGGCTG |
| | | | | TCAGGCCAGCCACCTGCAGTTCCCAGGGGC |
| | | | | TGTCTACGGCACAGATGGATGCCCTGTGTCG |
| | | | | GTCGAGAAGATTGTGAACATCTTCAATGGG |
| | | | | ACCAGCTGCCCCAGCCTGGGAGGGAAGCCC |
| | | | | AAGCTCTTTTTCATCCAGGCCTGTGGTGGGG |
| | | | | AGCAGAAAGACCATGGGTTTGAGGTGGCCT |
| | | | | CCACTTCCCCTGAAGACGAGTCCCCTGGCA |
| | | | | GTAACCCCGAGCCAGATGCCACCCCGTTCC |
| | | | | AGGAAGGTTTGAGGACCTTCGACCAGCTGG |
| | | | | ACGCCATATCTAGTTTGCCCACACCCAGTGA |
| | | | | CATCTTTGTGTCCTACTCTACTTTCCCAGGTT |
| | | | | TTGTTTCCTGGAGGGACCCCAAGAGTGGCTC |
| | | | | CTGGTACGTTGAGACCCTGGACGACATCTTT |
| | | | | GAGCAGTGGGCTCACTCTGAAGACCTGCAG |
| | | | | TCCCTCCTGCTTAGGGTCGCTAATGCTGTTT |
| | | | | CGGTGAAAGGGATTTATAAACAGATGCCTG |
| | | | | GTTGCTTTAATTTCCTCCGGAAAAAACTTTT |
| | | | | CTTTAAAACATCA |
| Bid/ | 54 | DCEVNNGSSLRDECITNLLVFGFLQSCSDN | 153 | GACTGTGAGGTCAACAACGGTTCCAGCCTC |
| truncated BID | | SFRRELDALGHELPVLAPQWEGYDELQTD | | AGGGATGAGTGCATCACAAACCTACTGGTG |
| (Tbid) | | GNRSSHSRLGRIEADSESQEDIIRNIARHLA | | TTTGGCTTCCTCCAAAGCTGTTCTGACAACA |
| | | QVGDSMDRSIPPGLVNGLALQLRNTSRSE | | GCTTCCGCAGAGAGCTGGACGCACTGGGCC |
| | | EDRNRDLATALEQLLQAYPRDMEKEKTM | | ACGAGCTGCCAGTGCTGGCTCCCCAGTGGG |
| | | LVLALLLAKKVASHTPSLLRDVFHTTVNFI | | AGGGCTACGATGAGCTGCAGACTGATGGCA |
| | | NQNLRTYVRSLARNGMD | | ACCGCAGCAGCCACTCCCGCTTGGGAAGAA |
| | | | | TAGAGGCAGATTCTGAAAGTCAAGAAGACA |
| | | | | TCATCCGGAATATTGCCAGGCACCTCGCCCA |
| | | | | GGTCGGGGACAGCATGGACCGTAGCATCCC |
| | | | | TCCGGGCCTGGTGAACGGCCTGGCCCTGCA |
| | | | | GCTCAGGAACACCAGCCGGTCGGAGGAGGA |
| | | | | CCGGAACAGGGACCTGGCCACTGCCCTGGA |
| | | | | GCAGCTGCTGCAGGCCTACCCTAGAGACAT |
| | | | | GGAGAAGGAGAAGACCATGCTGGTGCTGGC |
| | | | | CCTGCTGCTGGCCAAGAAGGTGGCCAGTCA |
| | | | | CACGCCGTCCTTGCTCCGTGATGTCTTTCAC |
| | | | | ACAACAGTGAATTTTATTAACCAGAACCTA |
| | | | | CGCACCTACGTGAGGAGCTTAGCCAGAAAT |
| | | | | GGGATGGAC |
| VPR | 55 | EASGSGRADALDDFDLDMLGSDALDDFD | 82 | GAAGCCTCTGGAAGCGGCAGAGCTGACGCC |
| | | LDMLGSDALDDFDLDMLGSDALDDFDLD | | CTGGATGACTTCGACCTGGATATGCTGGGC |
| | | MLINSRSSGSPKKKRKVGSQYLPDTDDRH | | AGCGACGCTCTGGACGATTTTGACCTCGAC |
| | | RIEEKRKRTYETFKSIMKKSPFSGPTDPRPP | | ATGCTGGGATCTGATGCACTCGACGATTTCG |
| | | PRRIAVPSRSSASVPKPAPQPYPFTSSLSTI | | ATTTGGACATGCTCGGCAGTGATGCCTTGGA |
| | | NYDEFPTMVFPSGQISQASALAPAPPQVLPQ | | CGACTTTGATCTTGATATGCTCATCAACAGC |
| | | APAPAPAPAMVSALAQAPAPVPVLAPGPP | | CGGTCCAGCGGCAGCCCCAAGAAAAAAAGA |
| | | QAVAPPAPKPTQAGEGTLSEALLQLQFDD | | AAAGTGGGCTCCCAGTACCTGCCTGACACC |
| | | EDLGALLGNSTDPAVFTDLASVDNSEFQQ | | GACGACAGACACCGGATCGAGGAAAAGCG |
| | | LLNQGIPVAPHTTEPMLMEYPEAITRLVTG | | GAAGCGGACCTACGAGACATTCAAGAGCAT |
| | | AQRPPDPAPAPLGAPGLPNGLLSGDEDESS | | CATGAAGAAGTCCCCATTCAGCGGCCCCAC |
| | | LADMDFSALLGSGSGSRDSREGMFLPKPE | | CGATCCTAGACCTCCACCTAGAAGAATCGC |
| | | AGSAISDVFEGREVCQPKRIRPFHPPGSPW | | CGTGCCTAGCAGATCTAGCGCCTCCGTGCCT |
| | | ANRPLPASLAPTPTGPVHEPVGSLTPAPVP | | AAACCTGCTCCTCAGCCTTATCCTTTCACCA |
| | | QPLDPAPAVTPEASHLLEDPDEETSQAVK | | GCAGCCTGAGCACCATCAACTACGACGAGT |
| | | ALREMADTVIPQKEEAAICGQMDLSHPPP | | TCCCTACCATGGTGTTCCCCAGCGGCCAGAT |
| | | RGHLDELTTTLESMTEDLNLDSPLTPELNE | | CTCTCAGGCTTCTGCTCTTGCTCCAGCTCCT |
| | | ILDTFLNDECLLHAMHISTGLSIFDTSLF | | CCTCAGGTTCTGCCTCAAGCTCCTGCACCAG |
| | | | | CACCGGCTCCAGCTATGGTTTCTGCTTTGGC |
| | | | | TCAGGCCCCTGCTCCTGTGCCTGTTCTTGCT |
| | | | | CCTGGACCACCTCAGGCTGTTGCTCCTCCTG |
| | | | | CTCCAAAACCTACACAGGCCGGCGAAGGCA |
| | | | | CACTGTCTGAAGCTCTGCTGCAGCTCCAGTT |
| | | | | CGATGACGAAGATCTGGGCGCCCTGCTGGG |
| | | | | CAATTCTACAGATCCTGCCGTGTTTACCGAT |
| | | | | CTGGCCAGCGTGGACAACAGCGAGTTTCAG |
| | | | | CAGCTCCTGAATCAGGGCATCCCTGTGGCTC |
| | | | | CTCACACCACCGAACCTATGCTGATGGAAT |
| | | | | ACCCCGAGGCCATCACCAGACTGGTCACCG |
| | | | | GTGCTCAAAGACCACCTGATCCAGCTCCAG |
| | | | | CACCACTGGGAGCACCTGGACTGCCTAATG |
| | | | | GACTGCTGTCTGGCGACGAGGACTTCAGCT |
| | | | | CTATCGCCGACATGGATTTCTCTGCCCTGCT |
| | | | | CGGCTCTGGCAGCGGCTCTAGAGATAGCAG |
| | | | | AGAAGGCATGTTCCTGCCTAAGCCTGAGGC |
| | | | | CGGCTCTGCCATCTCCGATGTGTTCGAGGGA |
| | | | | AGAGAAGTGTGCCAGCCTAAGCGGATCCGG |
| | | | | CCTTTTCACCCTCCTGGAAGCCCTTGGGCCA |
| | | | | ACAGACCTCTGCCTGCTTCTCTGGCCCCTAC |
| | | | | ACCAACAGGACCTGTGCACGAACCTGTGGG |
| | | | | CAGTCTGACCCCAGCTCCTGTTCCTCAACCT |
| | | | | CTGGATCCCGCTCCTGCTGTGACACCTGAAG |
| | | | | CCTCTCATCTGCTGGAAGATCCCGACGAAG |
| | | | | AGACAAGCCAGGCCGTGAAGGCCCTGAGAG |
| | | | | AAATGGCCGACACAGTGATCCCTCAGAAAG |
| | | | | AGGAAGCCGCCATCTGCGGACAGATGGACC |
| | | | | TGTCTCATCCTCCACCAAGAGGCCACCTGGA |
| | | | | CGAGCTGACAACCACACTGGAATCCATGAC |
| | | | | CGAGGACCTGAACCTGGACAGCCCTCTGAC |
| | | | | ACCCGAGCTGAACGAGATCCTGGACACCTT |
| | | | | CCTGAACGACGAGTGTCTGCTGCACGCCAT |
| | | | | GCACATCTCTACCGGCCTGAGCATCTTCGAC |
| | | | | ACCAGCCTGTTT |
| XIAP (with | 56 | SRGSEFMTFNSFEGSKTCVPADINKEEEFV | 83 | TCTAGAGGATCCGAATTCATGACTTTTAACA |
| N-terminal | | EEFNRLKTFANFPSGSPVSASTLARAGFLY | | GTTTTGAAGGATCTAAAACTTGTGTACCTGC |
| modification) | | TGEGDTVRCFSCHAAVDRWQYGDSAVGR | | AGACATCAATAAGGAAGAAGAATTTGTAGA |
| | | HRKVSPNCRFINGFYLENSATQSTNSGIQN | | AGAGTTTAATAGATTAAAAACTTTTGCTAAT |
| | | GQYKVENYLGSRDHFALDRPSETHADYL | | TTTCCAAGTGGTAGTCCTGTTTCAGCATCAA |
| | | LRTGQVVDISDTIYPRNPAMYSEEARLKSF | | CACTGGCACGAGCAGGGTTTCTTTATACTGG |
| | | QNWPDYAHLTPRELASAGLYYTGIGDQV | | TGAAGGAGATACCGTGCGGTGCTTTAGTTGT |
| | | QCFCCGGKLKNWEPCDRAWSEHRRHFPN | | CATGCAGCTGTAGATAGGTGGCAATATGGA |
| | | CFFVLGRNLNIRSESDAVSSDRNFPNSTNL | | GACTCAGCAGTTGGAAGACACAGGAAAGTA |
| | | PRNPSMADYEARIFTFGTWIYSVNKEQLA | | TCCCCAAATTGCAGATTTATCAACGGCTTTT |
| | | RAGFYALGEGDKVKCFHCGGGLTDWKPS | | ATCTTGAAAATAGTGCCACGCAGTCTACAA |
| | | EDPWEQHAKWYPGCKYLLEQKGQEYINN | | ATTCTGGTATCCAGAATGGTCAGTACAAAG |
| | | IHLTHSLEECLVRTTEKTPSLTRRIDDTIFQ | | TTGAAAACTATCTGGGAAGCAGAGATCATT |
| | | NPMVQEAIRMGFSFKDIKKIMEEKIQISGS | | TTGCCTTAGACAGGCCATCTGAGACACATG |
| | | NYKSLEVLVADLVNAQKDSMQDESSQTS | | CAGACTATCTTTTGAGAACTGGGCAGGTTGT |
| | | LQKEISTEEQLRRLQEEKLCKICMDRNIAI | | AGATATATCAGACACCATATACCCGAGGAA |
| | | VFVPCGHLVTCKQCAEAVDKCPMCYTVI | | CCCTGCCATGTATAGTGAAGAAGCTAGATT |
| | | TFKQKIFMS | | AAAGTCCTTTCAGAACTGGCCAGACTATGCT |
| | | | | CACCTAACCCCAAGAGAGTTAGCAAGTGCT |
| | | | | GGACTCTACTACACAGGTATTGGTGACCAA |
| | | | | GTGCAGTGCTTTTGTTGTGGTGGAAAACTGA |
| | | | | AAAATTGGGAACCTTGTGATCGTGCCTGGTC |
| | | | | AGAACACAGGCGACACTTTCCTAATTGCTTC |
| | | | | TTTGTTTTGGGCCGGAATCTTAATATTCGAA |
| | | | | GTGAATCTGATGCTGTGAGTTCTGATAGGA |
| | | | | ATTTCCCAAATTCAACAAATCTTCCAAGAAA |
| | | | | TCCATCCATGGCAGATTATGAAGCACGGAT |
| | | | | CTTTACTTTTGGGACATGGATATACTCAGTT |
| | | | | AACAAGGAGCAGCTTGCAAGAGCTGGATTT |
| | | | | TATGCTTTAGGTGAAGGTGATAAAGTAAAG |
| | | | | TGCTTTCACTGTGGAGGAGGGCTAACTGATT |
| | | | | GGAAGCCCAGTGAAGACCCTTGGGAACAAC |
| | | | | ATGCTAAATGGTATCCAGGGTGCAAATATC |
| | | | | TGTTAGAACAGAAGGGACAAGAATATATAA |
| | | | | ACAATATTCATTTAACTCATTCACTTGAGGA |
| | | | | GTGTCTGGTAAGAACTACTGAGAAAACACC |
| | | | | ATCACTAACTAGAAGAATTGATGATACCAT |
| | | | | CTTCCAAAATCCTATGGTACAAGAAGCTAT |
| | | | | ACGAATGGGGTTCAGTTTCAAGGACATTAA |
| | | | | GAAAATAATGGAGGAAAAAATTCAGATATC |
| | | | | TGGGAGCAACTATAAATCACTTGAGGTTCT |
| | | | | GGTTGCAGATCTAGTGAATGCTCAGAAAGA |
| | | | | CAGTATGCAAGATGAGTCAAGTCAGACTTC |
| | | | | ATTACAGAAAGAGATTAGTACTGAAGAGCA |
| | | | | GCTAAGGCGCCTGCAAGAGGAGAAGCTTTG |
| | | | | CAAAATCTGTATGGATAGAAATATTGCTATC |
| | | | | GTTTTTGTTCCTTGTGGACATCTAGTCACTT |
| | | | | GTAAACAATGTGCTGAAGCAGTTGACAAGT |
| | | | | GTCCCATGTGCTACACAGTCATTACTTTCAA |
| | | | | GCAAAAAATTTTTATGTCT |
| ZF10-1 | 57 | SRPGERPFQCRICMRNFSRRHGLDRHTRT | 84 | TCTAGACCCGGCGAAAGACCCTTCCAGTGC |
| | | HTGEKPFQCRICMRNFSDHSSLKRHLRTH | | CGGATCTGCATGCGGAACTTCAGCAGAAGG |
| | | TGSQKPFQCRICMRNFSVRHNLTRHLRTH | | CACGGCCTGGACAGACACACCAGAACACAC |
| | | TGEKPFQCRICMRNFSDHSNLSRHLKTHT | | ACAGGCGAGAAGCCTTTCCAGTGTAGAATC |
| | | GSQKPFQCRICMRNFSQRSSLVRHLRTHT | | TGTATGCGCAATTTCAGCGACCACAGCAGC |
| | | GEKPFQCRICMRNFSESGHLKRHLRTHLR | | CTGAAGCGGCACCTGAGAACCCATACCGGC |
| | | GS | | AGCCAGAAACCATTTCAATGCCGCATCTGT |
| | | | | ATGAGAAACTTCTCCGTGCGGCACAACCTG |
| | | | | ACCAGACACCTGAGGACACACACCGGGGAG |
| | | | | AAACCCTTTCAGTGCAGAATATGCATGAGG |
| | | | | AATTTCTCCGACCACTCCAACCTGAGCCGCC |
| | | | | ACCTGAAAACTCACACCGGCTCTCAAAAGC |
| | | | | CATTTCAGTGTCGTATATGTATGCGGAATTT |
| | | | | TTCCCAGCGGAGCAGCCTCGTGCGCCATCTG |
| | | | | AGGACTCATACTGGCGAAAAGCCCTTCCAA |
| | | | | TGTCGCATATGCATGCGCAACTTTAGCGAGT |
| | | | | CCGGCCACCTGAAGAGACATCTGCGGACAC |
| | | | | ACCTGAGAGGCTCT |
| ZF10-1 | 84 | MPGERPFQCRICMRNFSRRHGLDRHTRTH | 85 | ATGCCCGGAGAGCGCCCATTCCAGTGTCGG |
| (alternative as | | TGEKPFQCRICMRNFSDHSSLKRHLRTHT | | ATTTGCATGCGGAACTTTTCGAGAAGACAC |
| used in | | GSQKPFQCRICMRNFSVRHNLTRHLRTHT | | GGCCTGGACAGACATACCCGTACTCATACA |
| Example 9) | | GEKPFQCRICMRNFSDHSNLSRHLKTHTG | | GGTGAAAAACCCTTTCAGTGTCGGATCTGTA |
| | | SQKPFQCRICMRNFSQRSSLVRHLRTHTGE | | TGCGAAATTTCTCCGACCACAGCAGCCTGA |
| | | KPFQCRICMRNFSESGHLKRHLRTHLRGS | | AGAGACATCTACGTACCCACACCGGCAGCC |
| | | | | AGAAGCCATTTCAGTGTCGGATCTGTATGCG |
| | | | | GAACTTCTCCGTGAGACACAACCTGACCAG |
| | | | | ACATCTACGTACGCACACCGGAGAGAAGCC |
| | | | | ATTCCAATGCCGAATATGCATGCGCAACTTC |
| | | | | AGTGACCACAGCAACCTGAGCAGACACCTA |
| | | | | AAAACCCACACCGGTTCCCAGAAGCCATTT |
| | | | | CAGTGTCGGATCTGTATGCGGAACTTCTCCC |
| | | | | AGCGCAGCAGCCTGGTGAGACATCTACGTA |
| | | | | CGCACACCGGAGAGAAGCCATTCCAATGCC |
| | | | | GAATATGCATGCGCAACTTCAGTGAGAGCG |
| | | | | GCCACCTGAAGAGACACCTGCGTACGCACC |
| | | | | TGAGGGGATCC |
| XIAP | 107 | MTFNSFEGSKTCVPADINKEEEFVEEFNRL | 108 | ATGACTTTTAACAGTTTTGAAGGATCTAAAA |
| | | KTFANFPSGSPVSASTLARAGFLYTGEGDT | | CTTGTGTACCTGCAGACATCAATAAGGAAG |
| | | VRCFSCHAAVDRWQYGDSAVGRHRKVSP | | AAGAATTTGTAGAAGAGTTTAATAGATTAA |
| | | NCRFINGFYLENSATQSTNSGIQNGQYKV | | AAACTTTTGCTAATTTTCCAAGTGGTAGTCC |
| | | ENYLGSRDHFALDRPSETHADYLLRTGQV | | TGTTTCAGCATCAACACTGGCACGAGCAGG |
| | | VDISDTIYPRNPAMYSEEARLKSFQNWPD | | GTTTCTTTATACTGGTGAAGGAGATACCGTG |
| | | YAHLTPRELASAGLYYTGIGDQVQCFCCG | | CGGTGCTTTAGTTGTCATGCAGCTGTAGATA |
| | | GKLKNWEPCDRAWSEHRRHFPNCFFVLG | | GGTGGCAATATGGAGACTCAGCAGTTGGAA |
| | | RNLNIRSESDAVSSDRNFPNSTNLPRNPSM | | GACACAGGAAAGTATCCCCAAATTGCAGAT |
| | | ADYEARIFTFGTWIYSVNKEQLARAGFYA | | TTATCAACGGCTTTTATCTTGAAAATAGTGC |
| | | LGEGDKVKCFHCGGGLTDWKPSEDPWEQ | | CACGCAGTCTACAAATTCTGGTATCCAGAAT |
| | | HAKWYPGCKYLLEQKGQEYINNIHLTHSL | | GGTCAGTACAAAGTTGAAAACTATCTGGGA |
| | | EECLVRTTEKTPSLTRRIDDTIFQNPMVQE | | AGCAGAGATCATTTTGCCTTAGACAGGCCA |
| | | AIRMGFSFKDIKKIMEEKIQISGSNYKSLEV | | TCTGAGACACATGCAGACTATCTTTTGAGAA |
| | | LVADLVNAQKDSMQDESSQTSLQKEISTE | | CTGGGCAGGTTGTAGATATATCAGACACCA |
| | | EQLRRLQEEKLCKICMDRNIAIVFVPCGHL | | TATACCCGAGGAACCCTGCCATGTATAGTG |
| | | VTCKQCAEAVDKCPMCYTVITFKQKIFMS | | AAGAAGCTAGATTAAAGTCCTTTCAGAACT |
| | | | | GGCCAGACTATGCTCACCTAACCCCAAGAG |
| | | | | AGTTAGCAAGTGCTGGACTCTACTACACAG |
| | | | | GTATTGGTGACCAAGTGCAGTGCTTTTGTTG |
| | | | | TGGTGGAAAACTGAAAAATTGGGAACCTTG |
| | | | | TGATCGTGCCTGGTCAGAACACAGGCGACA |
| | | | | CTTTCCTAATTGCTTCTTTGTTTTGGGCCGGA |
| | | | | ATCTTAATATTCGAAGTGAATCTGATGCTGT |
| | | | | GAGTTCTGATAGGAATTTCCCAAATTCAACA |
| | | | | AATCTTCCAAGAAATCCATCCATGGCAGATT |
| | | | | ATGAAGCACGGATCTTTACTTTTGGGACATG |
| | | | | GATATACTCAGTTAACAAGGAGCAGCTTGC |
| | | | | AAGAGCTGGATTTTATGCTTTAGGTGAAGGT |
| | | | | GATAAAGTAAAGTGCTTTCACTGTGGAGGA |
| | | | | GGGCTAACTGATTGGAAGCCCAGTGAAGAC |
| | | | | CCTTGGGAACAACATGCTAAATGGTATCCA |
| | | | | GGGTGCAAATATCTGTTAGAACAGAAGGGA |
| | | | | CAAGAATATATAAACAATATTCATTTAACTC |
| | | | | ATTCACTTGAGGAGTGTCTGGTAAGAACTA |
| | | | | CTGAGAAAACACCATCACTAACTAGAAGAA |
| | | | | TTGATGATACCATCTTCCAAAATCCTATGGT |
| | | | | ACAAGAAGCTATACGAATGGGGTTCAGTTT |
| | | | | CAAGGACATTAAGAAAATAATGGAGGAAA |
| | | | | AAATTCAGATATCTGGGAGCAACTATAAAT |
| | | | | CACTTGAGGTTCTGGTTGCAGATCTAGTGAA |
| | | | | TGCTCAGAAAGACAGTATGCAAGATGAGTC |
| | | | | AAGTCAGACTTCATTACAGAAAGAGATTAG |
| | | | | TACTGAAGAGCAGCTAAGGCGCCTGCAAGA |
| | | | | GGAGAAGCTTTGCAAAATCTGTATGGATAG |
| | | | | AAATATTGCTATCGTTTTTGTTCCTTGTGGA |
| | | | | CATCTAGTCACTTGTAAACAATGTGCTGAAG |
| | | | | CAGTTGACAAGTGTCCCATGTGCTACACAGT |
| | | | | CATTACTTTCAAGCAAAAAATTTTTATGTCT |
| XIAP (T308S, | 109 | MTFNSFEGSKTCVPADINKEEEFVEEFNRL | 110 | ATGACTTTTAACAGTTTTGAAGGATCTAAAA |
| G306S, | | KTFANFPSGSPVSASTLARAGFLYTGEGDT | | CTTGTGTACCTGCAGACATCAATAAGGAAG |
| G305M, | | VRCFSCHAAVDRWQYGDSAVGRHRKVSP | | AAGAATTTGTAGAAGAGTTTAATAGATTAA |
| P325S) | | NCRFINGFYLENSATQSTNSGIQNGQYKV | | AAACTTTTGCTAATTTTCCAAGTGGTAGTCC |
| | | ENYLGSRDHFALDRPSETHADYLLRTGQV | | TGTTTCAGCATCAACACTGGCACGAGCAGG |
| | | VDISDTIYPRNPAMYSEEARLKSFQNWPD | | GTTTCTTTATACTGGTGAAGGAGATACCGTG |
| | | YAHLTPRELASAGLYYTGIGDQVQCFCCG | | CGGTGCTTTAGTTGTCATGCAGCTGTAGATA |
| | | GKLKNWEPCDRAWSEHRRHFPNCFFVLG | | GGTGGCAATATGGAGACTCAGCAGTTGGAA |
| | | RNLNIRSESDAVSSDRNFPNSTNLPRNPSM | | GACACAGGAAAGTATCCCCAAATTGCAGAT |
| | | ADYEARIFTFGTWIYSVNKEQLARAGFYA | | TTATCAACGGCTTTTATCTTGAAAATAGTGC |
| | | LGEGDKVKCFHCGMSLSDWKPSEDPWEQ | | CACGCAGTCTACAAATTCTGGTATCCAGAAT |
| | | HAKWYSGCKYLLEQKGQEYINNIHLTHSL | | GGTCAGTACAAAGTTGAAAACTATCTGGGA |
| | | EECLVRTTEKTPSLTRRIDDTIFQNPMVQE | | AGCAGAGATCATTTTGCCTTAGACAGGCCA |
| | | AIRMGFSFKDIKKIMEEKIQISGSNYKSLEV | | TCTGAGACACATGCAGACTATCTTTTGAGAA |
| | | LVADLVNAQKDSMQDESSQTSLQKEISTE | | CTGGGCAGGTTGTAGATATATCAGACACCA |
| | | EQLRRLQEEKLCKICMDRNIAIVFVPCGHL | | TATACCCGAGGAACCCTGCCATGTATAGTG |
| | | VTCKQCAEAVDKCPMCYTVITFKQKIFMS | | AAGAAGCTAGATTAAAGTCCTTTCAGAACT |
| | | | | GGCCAGACTATGCTCACCTAACCCCAAGAG |
| | | | | AGTTAGCAAGTGCTGGACTCTACTACACAG |
| | | | | GTATTGGTGACCAAGTGCAGTGCTTTTGTTG |
| | | | | TGGTGGAAAACTGAAAAATTGGGAACCTTG |
| | | | | TGATCGTGCCTGGTCAGAACACAGGCGACA |
| | | | | CTTTCCTAATTGCTTCTTTGTTTTGGGCCGGA |
| | | | | ATCTTAATATTCGAAGTGAATCTGATGCTGT |
| | | | | GAGTTCTGATAGGAATTTCCCAAATTCAACA |
| | | | | AATCTTCCAAGAAATCCATCCATGGCAGATT |
| | | | | ATGAAGCACGGATCTTTACTTTTGGGACATG |
| | | | | GATATACTCAGTTAACAAGGAGCAGCTTGC |
| | | | | AAGAGCTGGATTTTATGCTTTAGGTGAAGGT |
| | | | | GATAAAGTAAAGTGCTTTCACTGTGGAatgag |
| | | | | cCTAagcGATTGGAAGCCCAGTGAAGACCCTT |
| | | | | GGGAACAACATGCTAAATGGTATagcGGGTG |
| | | | | CAAATATCTGTTAGAACAGAAGGGACAAGA |
| | | | | ATATATAAACAATATTCATTTAACTCATTCA |
| | | | | CTTGAGGAGTGTCTGGTAAGAACTACTGAG |
| | | | | AAAACACCATCACTAACTAGAAGAATTGAT |
| | | | | GATACCATCTTCCAAAATCCTATGGTACAAG |
| | | | | AAGCTATACGAATGGGGTTCAGTTTCAAGG |
| | | | | ACATTAAGAAAATAATGGAGGAAAAAATTC |
| | | | | AGATATCTGGGAGCAACTATAAATCACTTG |
| | | | | AGGTTCTGGTTGCAGATCTAGTGAATGCTCA |
| | | | | GAAAGACAGTATGCAAGATGAGTCAAGTCA |
| | | | | GACTTCATTACAGAAAGAGATTAGTACTGA |
| | | | | AGAGCAGCTAAGGCGCCTGCAAGAGGAGA |
| | | | | AGCTTTGCAAAATCTGTATGGATAGAAATA |
| | | | | TTGCTATCGTTTTTGTTCCTTGTGGACATCTA |
| | | | | GTCACTTGTAAACAATGTGCTGAAGCAGTT |
| | | | | GACAAGTGTCCCATGTGCTACACAGTCATTA |
| | | | | CTTTCAAGCAAAAAATTTTTATGTCT |
| XIAP | 111 | MTFNSFEGSKTCVPADINKEEEFVEEFNRL | 112 | ATGACTTTTAACAGTTTTGAAGGATCTAAAA |
| (T308D, | | KTFANFPSGSPVSASTLARAGFLYTGEGDT | | CTTGTGTACCTGCAGACATCAATAAGGAAG |
| G306S, | | VRCFSCHAAVDRWQYGDSAVGRHRKVSP | | AAGAATTTGTAGAAGAGTTTAATAGATTAA |
| G305M, | | NCRFINGFYLENSATQSTNSGIQNGQYKV | | AAACTTTTGCTAATTTTCCAAGTGGTAGTCC |
| P325S) | | ENYLGSRDHFALDRPSETHADYLLRTGQV | | TGTTTCAGCATCAACACTGGCACGAGCAGG |
| | | VDISDTIYPRNPAMYSEEARLKSFQNWPD | | GTTTCTTTATACTGGTGAAGGAGATACCGTG |
| | | YAHLTPRELASAGLYYTGIGDQVQCFCCG | | CGGTGCTTTAGTTGTCATGCAGCTGTAGATA |
| | | GKLKNWEPCDRAWSEHRRHFPNCFFVLG | | GGTGGCAATATGGAGACTCAGCAGTTGGAA |
| | | RNLNIRSESDAVSSDRNFPNSTNLPRNPSM | | GACACAGGAAAGTATCCCCAAATTGCAGAT |
| | | ADYEARIFTFGTWIYSVNKEQLARAGFYA | | TTATCAACGGCTTTTATCTTGAAAATAGTGC |
| | | LGEGDKVKCFHCGMSLDDWKPSEDPWEQ | | CACGCAGTCTACAAATTCTGGTATCCAGAAT |
| | | HAKWYSGCKYLLEQKGQEYINNIHLTHSL | | GGTCAGTACAAAGTTGAAAACTATCTGGGA |
| | | EECLVRTTEKTPSLTRRIDDTIFQNPMVQE | | AGCAGAGATCATTTTGCCTTAGACAGGCCA |
| | | AIRMGFSFKDIKKIMEEKIQISGSNYKSLEV | | TCTGAGACACATGCAGACTATCTTTTGAGAA |
| | | LVADLVNAQKDSMQDESSQTSLQKEISTE | | CTGGGCAGGTTGTAGATATATCAGACACCA |
| | | EQLRRLQEEKLCKICMDRNIAIVFVPCGHL | | TATACCCGAGGAACCCTGCCATGTATAGTG |
| | | VTCKQCAEAVDKCPMCYTVITFKQKIFMS | | AAGAAGCTAGATTAAAGTCCTTTCAGAACT |
| | | | | GGCCAGACTATGCTCACCTAACCCCAAGAG |
| | | | | AGTTAGCAAGTGCTGGACTCTACTACACAG |
| | | | | GTATTGGTGACCAAGTGCAGTGCTTTTGTTG |
| | | | | TGGTGGAAAACTGAAAAATTGGGAACCTTG |
| | | | | TGATCGTGCCTGGTCAGAACACAGGCGACA |
| | | | | CTTTCCTAATTGCTTCTTTGTTTTGGGCCGGA |
| | | | | ATCTTAATATTCGAAGTGAATCTGATGCTGT |
| | | | | GAGTTCTGATAGGAATTTCCCAAATTCAACA |
| | | | | AATCTTCCAAGAAATCCATCCATGGCAGATT |
| | | | | ATGAAGCACGGATCTTTACTTTTGGGACATG |
| | | | | GATATACTCAGTTAACAAGGAGCAGCTTGC |
| | | | | AAGAGCTGGATTTTATGCTTTAGGTGAAGGT |
| | | | | GATAAAGTAAAGTGCTTTCACTGTGGAatgag |
| | | | | cCTAgacGATTGGAAGCCCAGTGAAGACCCTT |
| | | | | GGGAACAACATGCTAAATGGTATagcGGGTG |
| | | | | CAAATATCTGTTAGAACAGAAGGGACAAGA |
| | | | | ATATATAAACAATATTCATTTAACTCATTCA |
| | | | | CTTGAGGAGTGTCTGGTAAGAACTACTGAG |
| | | | | AAAACACCATCACTAACTAGAAGAATTGAT |
| | | | | GATACCATCTTCCAAAATCCTATGGTACAAG |
| | | | | AAGCTATACGAATGGGGTTCAGTTTCAAGG |
| | | | | ACATTAAGAAAATAATGGAGGAAAAAATTC |
| | | | | AGATATCTGGGAGCAACTATAAATCACTTG |
| | | | | AGGTTCTGGTTGCAGATCTAGTGAATGCTCA |
| | | | | GAAAGACAGTATGCAAGATGAGTCAAGTCA |
| | | | | GACTTCATTACAGAAAGAGATTAGTACTGA |
| | | | | AGAGCAGCTAAGGCGCCTGCAAGAGGAGA |
| | | | | AGCTTTGCAAAATCTGTATGGATAGAAATA |
| | | | | TTGCTATCGTTTTTGTTCCTTGTGGACATCTA |
| | | | | GTCACTTGTAAACAATGTGCTGAAGCAGTT |
| | | | | GACAAGTGTCCCATGTGCTACACAGTCATTA |
| | | | | CTTTCAAGCAAAAAATTTTTATGTCT |
| XIAP | 113 | MTFNSFEGSKTCVPADINKEEEFVEEFNRL | 114 | ATGACTTTTAACAGTTTTGAAGGATCTAAAA |
| (T308S) | | KTFANFPSGSPVSASTLARAGFLYTGEGDT | | CTTGTGTACCTGCAGACATCAATAAGGAAG |
| | | VRCFSCHAAVDRWQYGDSAVGRHRKVSP | | AAGAATTTGTAGAAGAGTTTAATAGATTAA |
| | | NCRFINGFYLENSATQSTNSGIQNGQYKV | | AAACTTTTGCTAATTTTCCAAGTGGTAGTCC |
| | | ENYLGSRDHFALDRPSETHADYLLRTGQV | | TGTTTCAGCATCAACACTGGCACGAGCAGG |
| | | VDISDTIYPRNPAMYSEEARLKSFQNWPD | | GTTTCTTTATACTGGTGAAGGAGATACCGTG |
| | | YAHLTPRELASAGLYYTGIGDQVQCFCCG | | CGGTGCTTTAGTTGTCATGCAGCTGTAGATA |
| | | GKLKNWEPCDRAWSEHRRHFPNCFFVLG | | GGTGGCAATATGGAGACTCAGCAGTTGGAA |
| | | RNLNIRSESDAVSSDRNFPNSTNLPRNPSM | | GACACAGGAAAGTATCCCCAAATTGCAGAT |
| | | ADYEARIFTFGTWIYSVNKEQLARAGFYA | | TTATCAACGGCTTTTATCTTGAAAATAGTGC |
| | | LGEGDKVKCFHCGGGLSDWKPSEDPWEQ | | CACGCAGTCTACAAATTCTGGTATCCAGAAT |
| | | HAKWYPGCKYLLEQKGQEYINNIHLTHSL | | GGTCAGTACAAAGTTGAAAACTATCTGGGA |
| | | EECLVRTTEKTPSLTRRIDDTIFQNPMVQE | | AGCAGAGATCATTTTGCCTTAGACAGGCCA |
| | | AIRMGFSFKDIKKIMEEKIQISGSNYKSLEV | | TCTGAGACACATGCAGACTATCTTTTGAGAA |
| | | LVADLVNAQKDSMQDESSQTSLQKEISTE | | CTGGGCAGGTTGTAGATATATCAGACACCA |
| | | EQLRRLQEEKLCKICMDRNIAIVFVPCGHL | | TATACCCGAGGAACCCTGCCATGTATAGTG |
| | | VTCKQCAEAVDKCPMCYTVITFKQKIFMS | | AAGAAGCTAGATTAAAGTCCTTTCAGAACT |
| | | | | GGCCAGACTATGCTCACCTAACCCCAAGAG |
| | | | | AGTTAGCAAGTGCTGGACTCTACTACACAG |
| | | | | GTATTGGTGACCAAGTGCAGTGCTTTTGTTG |
| | | | | TGGTGGAAAACTGAAAAATTGGGAACCTTG |
| | | | | TGATCGTGCCTGGTCAGAACACAGGCGACA |
| | | | | CTTTCCTAATTGCTTCTTTGTTTTGGGCCGGA |
| | | | | ATCTTAATATTCGAAGTGAATCTGATGCTGT |
| | | | | GAGTTCTGATAGGAATTTCCCAAATTCAACA |
| | | | | AATCTTCCAAGAAATCCATCCATGGCAGATT |
| | | | | ATGAAGCACGGATCTTTACTTTTGGGACATG |
| | | | | GATATACTCAGTTAACAAGGAGCAGCTTGC |
| | | | | AAGAGCTGGATTTTATGCTTTAGGTGAAGGT |
| | | | | GATAAAGTAAAGTGCTTTCACTGTGGAGGA |
| | | | | GGGCTAagcGATTGGAAGCCCAGTGAAGACC |
| | | | | CTTGGGAACAACATGCTAAATGGTATCCAG |
| | | | | GGTGCAAATATCTGTTAGAACAGAAGGGAC |
| | | | | AAGAATATATAAACAATATTCATTTAACTCA |
| | | | | TTCACTTGAGGAGTGTCTGGTAAGAACTACT |
| | | | | GAGAAAACACCATCACTAACTAGAAGAATT |
| | | | | GATGATACCATCTTCCAAAATCCTATGGTAC |
| | | | | AAGAAGCTATACGAATGGGGTTCAGTTTCA |
| | | | | AGGACATTAAGAAAATAATGGAGGAAAAA |
| | | | | ATTCAGATATCTGGGAGCAACTATAAATCA |
| | | | | CTTGAGGTTCTGGTTGCAGATCTAGTGAATG |
| | | | | CTCAGAAAGACAGTATGCAAGATGAGTCAA |
| | | | | GTCAGACTTCATTACAGAAAGAGATTAGTA |
| | | | | CTGAAGAGCAGCTAAGGCGCCTGCAAGAGG |
| | | | | AGAAGCTTTGCAAAATCTGTATGGATAGAA |
| | | | | ATATTGCTATCGTTTTTGTTCCTTGTGGACAT |
| | | | | CTAGTCACTTGTAAACAATGTGCTGAAGCA |
| | | | | GTTGACAAGTGTCCCATGTGCTACACAGTCA |
| | | | | TTACTTTCAAGCAAAAAATTTTTATGTCT |
| XIAP | 115 | MTFNSFEGSKTCVPADINKEEEFVEEFNRL | 116 | ATGACTTTTAACAGTTTTGAAGGATCTAAAA |
| (T308D) | | KTFANFPSGSPVSASTLARAGFLYTGEGDT | | CTTGTGTACCTGCAGACATCAATAAGGAAG |
| | | VRCFSCHAAVDRWQYGDSAVGRHRKVSP | | AAGAATTTGTAGAAGAGTTTAATAGATTAA |
| | | NCRFINGFYLENSATQSTNSGIQNGQYKV | | AAACTTTTGCTAATTTTCCAAGTGGTAGTCC |
| | | ENYLGSRDHFALDRPSETHADYLLRTGQV | | TGTTTCAGCATCAACACTGGCACGAGCAGG |
| | | VDISDTIYPRNPAMYSEEARLKSFQNWPD | | GTTTCTTTATACTGGTGAAGGAGATACCGTG |
| | | YAHLTPRELASAGLYYTGIGDQVQCFCCG | | CGGTGCTTTAGTTGTCATGCAGCTGTAGATA |
| | | GKLKNWEPCDRAWSEHRRHFPNCFFVLG | | GGTGGCAATATGGAGACTCAGCAGTTGGAA |
| | | RNLNIRSESDAVSSDRNFPNSTNLPRNPSM | | GACACAGGAAAGTATCCCCAAATTGCAGAT |
| | | ADYEARIFTFGTWIYSVNKEQLARAGFYA | | TTATCAACGGCTTTTATCTTGAAAATAGTGC |
| | | LGEGDKVKCFHCGGGLDDWKPSEDPWEQ | | CACGCAGTCTACAAATTCTGGTATCCAGAAT |
| | | HAKWYPGCKYLLEQKGQEYINNIHLTHSL | | GGTCAGTACAAAGTTGAAAACTATCTGGGA |
| | | EECLVRTTEKTPSLTRRIDDTIFQNPMVQE | | AGCAGAGATCATTTTGCCTTAGACAGGCCA |
| | | AIRMGFSFKDIKKIMEEKIQISGSNYKSLEV | | TCTGAGACACATGCAGACTATCTTTTGAGAA |
| | | LVADLVNAQKDSMQDESSQTSLQKEISTE | | CTGGGCAGGTTGTAGATATATCAGACACCA |
| | | EQLRRLQEEKLCKICMDRNIAIVFVPCGHL | | TATACCCGAGGAACCCTGCCATGTATAGTG |
| | | VTCKQCAEAVDKCPMCYTVITFKQKIFMS | | AAGAAGCTAGATTAAAGTCCTTTCAGAACT |
| | | | | GGCCAGACTATGCTCACCTAACCCCAAGAG |
| | | | | AGTTAGCAAGTGCTGGACTCTACTACACAG |
| | | | | GTATTGGTGACCAAGTGCAGTGCTTTTGTTG |
| | | | | TGGTGGAAAACTGAAAAATTGGGAACCTTG |
| | | | | TGATCGTGCCTGGTCAGAACACAGGCGACA |
| | | | | CTTTCCTAATTGCTTCTTTGTTTTGGGCCGGA |
| | | | | ATCTTAATATTCGAAGTGAATCTGATGCTGT |
| | | | | GAGTTCTGATAGGAATTTCCCAAATTCAACA |
| | | | | AATCTTCCAAGAAATCCATCCATGGCAGATT |
| | | | | ATGAAGCACGGATCTTTACTTTTGGGACATG |
| | | | | GATATACTCAGTTAACAAGGAGCAGCTTGC |
| | | | | AAGAGCTGGATTTTATGCTTTAGGTGAAGGT |
| | | | | GATAAAGTAAAGTGCTTTCACTGTGGAGGA |
| | | | | GGGCTAgacGATTGGAAGCCCAGTGAAGACC |
| | | | | CTTGGGAACAACATGCTAAATGGTATCCAG |
| | | | | GGTGCAAATATCTGTTAGAACAGAAGGGAC |
| | | | | AAGAATATATAAACAATATTCATTTAACTCA |
| | | | | TTCACTTGAGGAGTGTCTGGTAAGAACTACT |
| | | | | GAGAAAACACCATCACTAACTAGAAGAATT |
| | | | | GATGATACCATCTTCCAAAATCCTATGGTAC |
| | | | | AAGAAGCTATACGAATGGGGTTCAGTTTCA |
| | | | | AGGACATTAAGAAAATAATGGAGGAAAAA |
| | | | | ATTCAGATATCTGGGAGCAACTATAAATCA |
| | | | | CTTGAGGTTCTGGTTGCAGATCTAGTGAATG |
| | | | | CTCAGAAAGACAGTATGCAAGATGAGTCAA |
| | | | | GTCAGACTTCATTACAGAAAGAGATTAGTA |
| | | | | CTGAAGAGCAGCTAAGGCGCCTGCAAGAGG |
| | | | | AGAAGCTTTGCAAAATCTGTATGGATAGAA |
| | | | | ATATTGCTATCGTTTTTGTTCCTTGTGGACAT |
| | | | | CTAGTCACTTGTAAACAATGTGCTGAAGCA |
| | | | | GTTGACAAGTGTCCCATGTGCTACACAGTCA |
| | | | | TTACTTTCAAGCAAAAAATTTTTATGTCT |
| XIAP | 117 | MTFNSFEGSKTCVPADINKEEEFVEEFNRL | 118 | ATGACTTTTAACAGTTTTGAAGGATCTAAAA |
| (G306S) | | KTFANFPSGSPVSASTLARAGFLYTGEGDT | | CTTGTGTACCTGCAGACATCAATAAGGAAG |
| | | VRCFSCHAAVDRWQYGDSAVGRHRKVSP | | AAGAATTTGTAGAAGAGTTTAATAGATTAA |
| | | NCRFINGFYLENSATQSTNSGIQNGQYKV | | AAACTTTTGCTAATTTTCCAAGTGGTAGTCC |
| | | ENYLGSRDHFALDRPSETHADYLLRTGQV | | TGTTTCAGCATCAACACTGGCACGAGCAGG |
| | | VDISDTIYPRNPAMYSEEARLKSFQNWPD | | GTTTCTTTATACTGGTGAAGGAGATACCGTG |
| | | YAHLTPRELASAGLYYTGIGDQVQCFCCG | | CGGTGCTTTAGTTGTCATGCAGCTGTAGATA |
| | | GKLKNWEPCDRAWSEHRRHFPNCFFVLG | | GGTGGCAATATGGAGACTCAGCAGTTGGAA |
| | | RNLNIRSESDAVSSDRNFPNSTNLPRNPSM | | GACACAGGAAAGTATCCCCAAATTGCAGAT |
| | | ADYEARIFTFGTWIYSVNKEQLARAGFYA | | TTATCAACGGCTTTTATCTTGAAAATAGTGC |
| | | LGEGDKVKCFHCGGSLTDWKPSEDPWEQ | | CACGCAGTCTACAAATTCTGGTATCCAGAAT |
| | | HAKWYPGCKYLLEQKGQEYINNIHLTHSL | | GGTCAGTACAAAGTTGAAAACTATCTGGGA |
| | | EECLVRTTEKTPSLTRRIDDTIFQNPMVQE | | AGCAGAGATCATTTTGCCTTAGACAGGCCA |
| | | AIRMGFSFKDIKKIMEEKIQISGSNYKSLEV | | TCTGAGACACATGCAGACTATCTTTTGAGAA |
| | | LVADLVNAQKDSMQDESSQTSLQKEISTE | | CTGGGCAGGTTGTAGATATATCAGACACCA |
| | | EQLRRLQEEKLCKICMDRNIAIVFVPCGHL | | TATACCCGAGGAACCCTGCCATGTATAGTG |
| | | VTCKQCAEAVDKCPMCYTVITFKQKIFMS | | AAGAAGCTAGATTAAAGTCCTTTCAGAACT |
| | | | | GGCCAGACTATGCTCACCTAACCCCAAGAG |
| | | | | AGTTAGCAAGTGCTGGACTCTACTACACAG |
| | | | | GTATTGGTGACCAAGTGCAGTGCTTTTGTTG |
| | | | | TGGTGGAAAACTGAAAAATTGGGAACCTTG |
| | | | | TGATCGTGCCTGGTCAGAACACAGGCGACA |
| | | | | CTTTCCTAATTGCTTCTTTGTTTTGGGCCGGA |
| | | | | ATCTTAATATTCGAAGTGAATCTGATGCTGT |
| | | | | GAGTTCTGATAGGAATTTCCCAAATTCAACA |
| | | | | AATCTTCCAAGAAATCCATCCATGGCAGATT |
| | | | | ATGAAGCACGGATCTTTACTTTTGGGACATG |
| | | | | GATATACTCAGTTAACAAGGAGCAGCTTGC |
| | | | | AAGAGCTGGATTTTATGCTTTAGGTGAAGGT |
| | | | | GATAAAGTAAAGTGCTTTCACTGTGGAGGAa |
| | | | | gcCTAACTGATTGGAAGCCCAGTGAAGACCC |
| | | | | TTGGGAACAACATGCTAAATGGTATCCAGG |
| | | | | GTGCAAATATCTGTTAGAACAGAAGGGACA |
| | | | | AGAATATATAAACAATATTCATTTAACTCAT |
| | | | | TCACTTGAGGAGTGTCTGGTAAGAACTACT |
| | | | | GAGAAAACACCATCACTAACTAGAAGAATT |
| | | | | GATGATACCATCTTCCAAAATCCTATGGTAC |
| | | | | AAGAAGCTATACGAATGGGGTTCAGTTTCA |
| | | | | AGGACATTAAGAAAATAATGGAGGAAAAA |
| | | | | ATTCAGATATCTGGGAGCAACTATAAATCA |
| | | | | CTTGAGGTTCTGGTTGCAGATCTAGTGAATG |
| | | | | CTCAGAAAGACAGTATGCAAGATGAGTCAA |
| | | | | GTCAGACTTCATTACAGAAAGAGATTAGTA |
| | | | | CTGAAGAGCAGCTAAGGCGCCTGCAAGAGG |
| | | | | AGAAGCTTTGCAAAATCTGTATGGATAGAA |
| | | | | ATATTGCTATCGTTTTTGTTCCTTGTGGACAT |
| | | | | CTAGTCACTTGTAAACAATGTGCTGAAGCA |
| | | | | GTTGACAAGTGTCCCATGTGCTACACAGTCA |
| | | | | TTACTTTCAAGCAAAAAATTTTTATGTCT |
| XIAP | 119 | MTFNSFEGSKTCVPADINKEEEFVEEFNRL | 120 | ATGACTTTTAACAGTTTTGAAGGATCTAAAA |
| (G305M) | | KTFANFPSGSPVSASTLARAGFLYTGEGDT | | CTTGTGTACCTGCAGACATCAATAAGGAAG |
| | | VRCFSCHAAVDRWQYGDSAVGRHRKVSP | | AAGAATTTGTAGAAGAGTTTAATAGATTAA |
| | | NCRFINGFYLENSATQSTNSGIQNGQYKV | | AAACTTTTGCTAATTTTCCAAGTGGTAGTCC |
| | | ENYLGSRDHFALDRPSETHADYLLRTGQV | | TGTTTCAGCATCAACACTGGCACGAGCAGG |
| | | VDISDTIYPRNPAMYSEEARLKSFQNWPD | | GTTTCTTTATACTGGTGAAGGAGATACCGTG |
| | | YAHLTPRELASAGLYYTGIGDQVQCFCCG | | CGGTGCTTTAGTTGTCATGCAGCTGTAGATA |
| | | GKLKNWEPCDRAWSEHRRHFPNCFFVLG | | GGTGGCAATATGGAGACTCAGCAGTTGGAA |
| | | RNLNIRSESDAVSSDRNFPNSTNLPRNPSM | | GACACAGGAAAGTATCCCCAAATTGCAGAT |
| | | ADYEARIFTFGTWIYSVNKEQLARAGFYA | | TTATCAACGGCTTTTATCTTGAAAATAGTGC |
| | | LGEGDKVKCFHCGMGLTDWKPSEDPWE | | CACGCAGTCTACAAATTCTGGTATCCAGAAT |
| | | QHAKWYPGCKYLLEQKGQEYINNIHLTHS | | GGTCAGTACAAAGTTGAAAACTATCTGGGA |
| | | LEECLVRTTEKTPSLTRRIDDTIFQNPMVQ | | AGCAGAGATCATTTTGCCTTAGACAGGCCA |
| | | EAIRMGFSFKDIKKIMEEKIQISGSNYKSLE | | TCTGAGACACATGCAGACTATCTTTTGAGAA |
| | | VLVADLVNAQKDSMQDESSQTSLQKEIST | | CTGGGCAGGTTGTAGATATATCAGACACCA |
| | | EEQLRRLQEEKLCKICMDRNIAIVFVPCGH | | TATACCCGAGGAACCCTGCCATGTATAGTG |
| | | LVTCKQCAEAVDKCPMCYTVITFKQKIFM | | AAGAAGCTAGATTAAAGTCCTTTCAGAACT |
| | | S | | GGCCAGACTATGCTCACCTAACCCCAAGAG |
| | | | | AGTTAGCAAGTGCTGGACTCTACTACACAG |
| | | | | GTATTGGTGACCAAGTGCAGTGCTTTTGTTG |
| | | | | TGGTGGAAAACTGAAAAATTGGGAACCTTG |
| | | | | TGATCGTGCCTGGTCAGAACACAGGCGACA |
| | | | | CTTTCCTAATTGCTTCTTTGTTTTGGGCCGGA |
| | | | | ATCTTAATATTCGAAGTGAATCTGATGCTGT |
| | | | | GAGTTCTGATAGGAATTTCCCAAATTCAACA |
| | | | | AATCTTCCAAGAAATCCATCCATGGCAGATT |
| | | | | ATGAAGCACGGATCTTTACTTTTGGGACATG |
| | | | | GATATACTCAGTTAACAAGGAGCAGCTTGC |
| | | | | AAGAGCTGGATTTTATGCTTTAGGTGAAGGT |
| | | | | GATAAAGTAAAGTGCTTTCACTGTGGAatgG |
| | | | | GGCTAACTGATTGGAAGCCCAGTGAAGACC |
| | | | | CTTGGGAACAACATGCTAAATGGTATCCAG |
| | | | | GGTGCAAATATCTGTTAGAACAGAAGGGAC |
| | | | | AAGAATATATAAACAATATTCATTTAACTCA |
| | | | | TTCACTTGAGGAGTGTCTGGTAAGAACTACT |
| | | | | GAGAAAACACCATCACTAACTAGAAGAATT |
| | | | | GATGATACCATCTTCCAAAATCCTATGGTAC |
| | | | | AAGAAGCTATACGAATGGGGTTCAGTTTCA |
| | | | | AGGACATTAAGAAAATAATGGAGGAAAAA |
| | | | | ATTCAGATATCTGGGAGCAACTATAAATCA |
| | | | | CTTGAGGTTCTGGTTGCAGATCTAGTGAATG |
| | | | | CTCAGAAAGACAGTATGCAAGATGAGTCAA |
| | | | | GTCAGACTTCATTACAGAAAGAGATTAGTA |
| | | | | CTGAAGAGCAGCTAAGGCGCCTGCAAGAGG |
| | | | | AGAAGCTTTGCAAAATCTGTATGGATAGAA |
| | | | | ATATTGCTATCGTTTTTGTTCCTTGTGGACAT |
| | | | | CTAGTCACTTGTAAACAATGTGCTGAAGCA |
| | | | | GTTGACAAGTGTCCCATGTGCTACACAGTCA |
| | | | | TTACTTTCAAGCAAAAAATTTTTATGTCT |
| XIAP | 121 | MTFNSFEGSKTCVPADINKEEEFVEEFNRL | 122 | ATGACTTTTAACAGTTTTGAAGGATCTAAAA |
| (P325S) | | KTFANFPSGSPVSASTLARAGFLYTGEGDT | | CTTGTGTACCTGCAGACATCAATAAGGAAG |
| | | VRCFSCHAAVDRWQYGDSAVGRHRKVSP | | AAGAATTTGTAGAAGAGTTTAATAGATTAA |
| | | NCRFINGFYLENSATQSTNSGIQNGQYKV | | AAACTTTTGCTAATTTTCCAAGTGGTAGTCC |
| | | ENYLGSRDHFALDRPSETHADYLLRTGQV | | TGTTTCAGCATCAACACTGGCACGAGCAGG |
| | | VDISDTIYPRNPAMYSEEARLKSFQNWPD | | GTTTCTTTATACTGGTGAAGGAGATACCGTG |
| | | YAHLTPRELASAGLYYTGIGDQVQCFCCG | | CGGTGCTTTAGTTGTCATGCAGCTGTAGATA |
| | | GKLKNWEPCDRAWSEHRRHFPNCFFVLG | | GGTGGCAATATGGAGACTCAGCAGTTGGAA |
| | | RNLNIRSESDAVSSDRNFPNSTNLPRNPSM | | GACACAGGAAAGTATCCCCAAATTGCAGAT |
| | | ADYEARIFTFGTWIYSVNKEQLARAGFYA | | TTATCAACGGCTTTTATCTTGAAAATAGTGC |
| | | LGEGDKVKCFHCGGGLTDWKPSEDPWEQ | | CACGCAGTCTACAAATTCTGGTATCCAGAAT |
| | | HAKWYSGCKYLLEQKGQEYINNIHLTHSL | | GGTCAGTACAAAGTTGAAAACTATCTGGGA |
| | | EECLVRTTEKTPSLTRRIDDTIFQNPMVQE | | AGCAGAGATCATTTTGCCTTAGACAGGCCA |
| | | AIRMGFSFKDIKKIMEEKIQISGSNYKSLEV | | TCTGAGACACATGCAGACTATCTTTTGAGAA |
| | | LVADLVNAQKDSMQDESSQTSLQKEISTE | | CTGGGCAGGTTGTAGATATATCAGACACCA |
| | | EQLRRLQEEKLCKICMDRNIAIVFVPCGHL | | TATACCCGAGGAACCCTGCCATGTATAGTG |
| | | VTCKQCAEAVDKCPMCYTVITFKQKIFMS | | AAGAAGCTAGATTAAAGTCCTTTCAGAACT |
| | | | | GGCCAGACTATGCTCACCTAACCCCAAGAG |
| | | | | AGTTAGCAAGTGCTGGACTCTACTACACAG |
| | | | | GTATTGGTGACCAAGTGCAGTGCTTTTGTTG |
| | | | | TGGTGGAAAACTGAAAAATTGGGAACCTTG |
| | | | | TGATCGTGCCTGGTCAGAACACAGGCGACA |
| | | | | CTTTCCTAATTGCTTCTTTGTTTTGGGCCGGA |
| | | | | ATCTTAATATTCGAAGTGAATCTGATGCTGT |
| | | | | GAGTTCTGATAGGAATTTCCCAAATTCAACA |
| | | | | AATCTTCCAAGAAATCCATCCATGGCAGATT |
| | | | | ATGAAGCACGGATCTTTACTTTTGGGACATG |
| | | | | GATATACTCAGTTAACAAGGAGCAGCTTGC |
| | | | | AAGAGCTGGATTTTATGCTTTAGGTGAAGGT |
| | | | | GATAAAGTAAAGTGCTTTCACTGTGGAGGA |
| | | | | GGGCTAACTGATTGGAAGCCCAGTGAAGAC |
| | | | | CCTTGGGAACAACATGCTAAATGGTATagcG |
| | | | | GGTGCAAATATCTGTTAGAACAGAAGGGAC |
| | | | | AAGAATATATAAACAATATTCATTTAACTCA |
| | | | | TTCACTTGAGGAGTGTCTGGTAAGAACTACT |
| | | | | GAGAAAACACCATCACTAACTAGAAGAATT |
| | | | | GATGATACCATCTTCCAAAATCCTATGGTAC |
| | | | | AAGAAGCTATACGAATGGGGTTCAGTTTCA |
| | | | | AGGACATTAAGAAAATAATGGAGGAAAAA |
| | | | | ATTCAGATATCTGGGAGCAACTATAAATCA |
| | | | | CTTGAGGTTCTGGTTGCAGATCTAGTGAATG |
| | | | | CTCAGAAAGACAGTATGCAAGATGAGTCAA |
| | | | | GTCAGACTTCATTACAGAAAGAGATTAGTA |
| | | | | CTGAAGAGCAGCTAAGGCGCCTGCAAGAGG |
| | | | | AGAAGCTTTGCAAAATCTGTATGGATAGAA |
| | | | | ATATTGCTATCGTTTTTGTTCCTTGTGGACAT |
| | | | | CTAGTCACTTGTAAACAATGTGCTGAAGCA |
| | | | | GTTGACAAGTGTCCCATGTGCTACACAGTCA |
| | | | | TTACTTTCAAGCAAAAAATTTTTATGTCT |
| Caspase-9 | 123 | MDEADRRLLRRCRLRLVEELQVDQLWDV | 124 | ATGGACGAAGCGGATCGGCGGCTCCTGCGG |
| (inclusive of | | LLSRELFRPHMIEDIQRAGSGSRRDQARQL | | CGGTGCCGGCTGCGGCTGGTGGAAGAGCTG |
| start codon | | IIDLETRGSQALPLFISCLEDTGQDMLASFL | | CAGGTGGACCAGCTCTGGGACGTCCTGCTG |
| and C- | | RTNRQAAKLSKPTLENLTPVVLRPEIRKPE | | AGCCGCGAGCTGTTCAGGCCCCATATGATC |
| terminal 6X | | VLRPETPRPVDIGSGGFGDVGALESLRGN | | GAGGACATCCAGCGGGCAGGCTCTGGATCT |
| histidine tag | | ADLAYILSMEPCGHCLIINNVNFCRESGLR | | CGGCGGGATCAGGCCAGGCAGCTGATCATA |
| (SEQ ID NO: | | TRTGSNIDCEKLRRRFSSLHFMVEVKGDL | | GATCTGGAGACTCGAGGGAGTCAGGCTCTT |
| 155)) | | TAKKMVLALLELARQDHGALDCCVVVIL | | CCTTTGTTCATCTCCTGCTTAGAGGACACAG |
| | | SHGCQASHLQFPGAVYGTDGCPVSVEKIV | | GCCAGGACATGCTGGCTTCGTTTCTGCGAAC |
| | | NIFNGTSCPSLGGKPKLFFIQACGGEQKDH | | TAACAGGCAAGCAGCAAAGTTGTCGAAGCC |
| | | GFEVASTSPEDESPGSNPEPDATPFQEGLR | | AACCCTAGAAAACCTTACCCCAGTGGTGCT |
| | | TFDQLDAISSLPTPSDIFVSYSTFPGFVSWR | | CAGACCAGAGATTCGCAAACCAGAGGTTCT |
| | | DPKSGSWYVETLDDIFEQWAHSEDLQSLL | | CAGACCGGAAACACCCAGACCAGTGGACAT |
| | | LRVANAVSVKGIYKQMPGCFNFLRKKLFF | | TGGTTCTGGAGGATTCGGTGATGTCGGTGCT |
| | | KTSHHHHHH | | CTTGAGAGTTTGAGGGGAAATGCAGATTTG |
| | | | | GCTTACATCCTGAGCATGGAGCCCTGTGGCC |
| | | | | ACTGCCTCATTATCAACAATGTGAACTTCTG |
| | | | | CCGTGAGTCCGGGCTCCGCACCCGCACTGG |
| | | | | CTCCAACATCGACTGTGAGAAGTTGCGGCG |
| | | | | TCGCTTCTCCTCGCTGCATTTCATGGTGGAG |
| | | | | GTGAAGGGCGACCTGACTGCCAAGAAAATG |
| | | | | GTGCTGGCTTTGCTGGAGCTGGCGCGGCAG |
| | | | | GACCACGGTGCTCTGGACTGCTGCGTGGTG |
| | | | | GTCATTCTCTCTCACGGCTGTCAGGCCAGCC |
| | | | | ACCTGCAGTTCCCAGGGGCTGTCTACGGCA |
| | | | | CAGATGGATGCCCTGTGTCGGTCGAGAAGA |
| | | | | TTGTGAACATCTTCAATGGGACCAGCTGCCC |
| | | | | CAGCCTGGGAGGGAAGCCCAAGCTCTTTTT |
| | | | | CATCCAGGCCTGTGGTGGGGAGCAGAAAGA |
| | | | | CCATGGGTTTGAGGTGGCCTCCACTTCCCCT |
| | | | | GAAGACGAGTCCCCTGGCAGTAACCCCGAG |
| | | | | CCAGATGCCACCCCGTTCCAGGAAGGTTTG |
| | | | | AGGACCTTCGACCAGCTGGACGCCATATCT |
| | | | | AGTTTGCCCACACCCAGTGACATCTTTGTGT |
| | | | | CCTACTCTACTTTCCCAGGTTTTGTTTCCTGG |
| | | | | AGGGACCCCAAGAGTGGCTCCTGGTACGTT |
| | | | | GAGACCCTGGACGACATCTTTGAGCAGTGG |
| | | | | GCTCACTCTGAAGACCTGCAGTCCCTCCTGC |
| | | | | TTAGGGTCGCTAATGCTGTTTCGGTGAAAGG |
| | | | | GATTTATAAACAGATGCCTGGTTGCTTTAAT |
| | | | | TTCCTCCGGAAAAAACTTTTCTTTAAAACAT |
| | | | | CACACCACCACCACCACCAC |
| iCasp-9 | 125 | MDVGALESLRGNADLAYILSMEPCGHCLI | 126 | ATGGATGTCGGTGCTCTTGAGAGTTTGAGG |
| (inclusive of | | INNVNFCRESGLRTRTGSNIDCEKLRRRFS | | GGAAATGCAGATTTGGCTTACATCCTGAGC |
| start codon) | | SLHFMVEVKGDLTAKKMVLALLELARQD | | ATGGAGCCCTGTGGCCACTGCCTCATTATCA |
| | | HGALDCCVVVILSHGCQASHLQFPGAVY | | ACAATGTGAACTTCTGCCGTGAGTCCGGGCT |
| | | GTDGCPVSVEKIVNIFNGTSCPSLGGKPKL | | CCGCACCCGCACTGGCTCCAACATCGACTGT |
| | | FFIQACGGEQKDHGFEVASTSPEDESPGSN | | GAGAAGTTGCGGCGTCGCTTCTCCTCGCTGC |
| | | PEPDATPFQEGLRTFDQLDAISSLPTPSDIF | | ATTTCATGGTGGAGGTGAAGGGCGACCTGA |
| | | VSYSTFPGFVSWRDPKSGSWYVETLDDIF | | CTGCCAAGAAAATGGTGCTGGCTTTGCTGG |
| | | EQWAHSEDLQSLLLRVANAVSVKGIYKQ | | AGCTGGCGCGGCAGGACCACGGTGCTCTGG |
| | | MPGCFNFLRKKLFFKTS | | ACTGCTGCGTGGTGGTCATTCTCTCTCACGG |
| | | | | CTGTCAGGCCAGCCACCTGCAGTTCCCAGG |
| | | | | GGCTGTCTACGGCACAGATGGATGCCCTGT |
| | | | | GTCGGTCGAGAAGATTGTGAACATCTTCAA |
| | | | | TGGGACCAGCTGCCCCAGCCTGGGAGGGAA |
| | | | | GCCCAAGCTCTTTTTCATCCAGGCCTGTGGT |
| | | | | GGGGAGCAGAAAGACCATGGGTTTGAGGTG |
| | | | | GCCTCCACTTCCCCTGAAGACGAGTCCCCTG |
| | | | | GCAGTAACCCCGAGCCAGATGCCACCCCGT |
| | | | | TCCAGGAAGGTTTGAGGACCTTCGACCAGC |
| | | | | TGGACGCCATATCTAGTTTGCCCACACCCAG |
| | | | | TGACATCTTTGTGTCCTACTCTACTTTCCCA |
| | | | | GGTTTTGTTTCCTGGAGGGACCCCAAGAGTG |
| | | | | GCTCCTGGTACGTTGAGACCCTGGACGACA |
| | | | | TCTTTGAGCAGTGGGCTCACTCTGAAGACCT |
| | | | | GCAGTCCCTCCTGCTTAGGGTCGCTAATGCT |
| | | | | GTTTCGGTGAAAGGGATTTATAAACAGATG |
| | | | | CCTGGTTGCTTTAATTTCCTCCGGAAAAAAC |
| | | | | TTTTCTTTAAAACATCA |
| Smac/ | 135 | MAALKSWLSRSVTSFFRYRQCLCVPVVA | 136 | ATGGCCGCTCTGAAGTCCTGGCTGAGCAGA |
| DIABLO | | NFKKRCFSELIRPWHKTVTIGFGVTLCAVP | | AGCGTGACCAGCTTCTTCCGGTACAGACAG |
| | | LAQKSEPHSLSSEALMRRAVSLVTDSTSTF | | TGCCTGTGCGTGCCCGTGGTGGCCAACTTCA |
| | | LSQTTYALIEAITEYTKAVYTLTSLYRQYT | | AGAAGAGATGCTTCAGCGAGCTGATCAGAC |
| | | SLLGKMNSEEEDEVWQVIIGARAEMTSKH | | CCTGGCACAAGACCGTGACCATCGGCTTTG |
| | | QEYLKLETTWMTAVGLSEMAAEAAYQT | | GCGTGACCCTGTGTGCCGTGCCTATCGCTCA |
| | | GADQASITARNHIQLVKLQVEEVHQLSRK | | GAAGTCTGAGCCTCACAGCCTGTCTAGCGA |
| | | AETKLAEAQIEELRQKTQEEGEERAESEQE | | GGCCCTTATGAGAAGGGCCGTGTCTCTGGTC |
| | | AYLRED | | ACCGACAGCACCAGCACATTTCTGAGCCAG |
| | | | | ACCACATACGCCCTGATCGAGGCCATCACC |
| | | | | GAGTACACCAAGGCCGTGTACACCCTGACC |
| | | | | AGCCTGTACCGGCAGTACACATCTCTGCTGG |
| | | | | GCAAGATGAACAGCGAGGAAGAGGACGAA |
| | | | | GTCTGGCAAGTGATCATCGGCGCCAGAGCC |
| | | | | GAGATGACCAGCAAGCACCAAGAGTACCTG |
| | | | | AAGCTGGAAACCACCTGGATGACAGCCGTG |
| | | | | GGCCTGTCTGAAATGGCCGCCGAAGCTGCT |
| | | | | TATCAGACCGGCGCTGATCAGGCCAGCATC |
| | | | | ACCGCCAGAAATCACATCCAGCTGGTCAAG |
| | | | | CTGCAGGTCGAGGAAGTGCACCAGCTGTCC |
| | | | | AGAAAGGCCGAGACAAAGCTGGCTGAGGCC |
| | | | | CAGATCGAGGAACTGCGGCAGAAAACCCAA |
| | | | | GAGGAAGGCGAGGAAAGAGCCGAGTCTGA |
| | | | | GCAAGAGGCCTACCTGAGAGAGGAC |

A cell death-inducing domain can include or be derived from Caspase 9, e.g., the amino acid sequence shown in SEQ ID NO: 39 or 123. A derivative of Caspase-9 includes an inducible Caspase-9 (“iCasp-9”), which is capable of inducing apoptosis due to drug-based dimerization, e.g., the amino acid sequence shown in SEQ ID NO: 48 or 125. In some embodiments, the capsase domain or derivative or functional fragment thereof, e.g., the inducible Caspase-9, does not include a Caspase Activation and Recruitment Domain (CARD) domain sequence.

A cell death-inducing domain can include BAX, e.g., the amino acid sequence shown in SEQ ID NO: 32.

### Regulatable Cell Survival Polypeptides

Inducible cell death systems can include a regulatable cell survival polypeptide that includes a modified estrogen receptor ligand binding domains (ER-LBD).

Exemplary cell survival polypeptides include one or more of XIAP, Bcl-2, Bcl-XI, Bcl-w, Bcl-2-related protein A1 (BCL2A1), Mcl-1, FLICE-like inhibitory protein (c-FLIP), and an adenoviral E1B-19K protein. A cell survival polypeptide can include XIAP. A cell survival polypeptide can include wild-type XIAP, e.g., having the amino acid sequence SEQ ID NO: 107. A cell survival polypeptide can include modified XIAP. A modified XIAP can include one or more amino acid substitutions with reference to SEQ ID NO: 107. A modified XIAP can include one or more amino acid substitutions within positions 305-325 with reference to SEQ ID NO: 107. A modified XIAP can include one or more amino acid substitutions including 305, 306, 308, or 325 with reference to SEQ ID NO: 107. A modified XIAP can include one or more amino acid substitutions including each of 305, 306, 308, and 325 with reference to SEQ ID NO: 107. A modified XIAP can include one or more amino acid substitutions including each of 305, 306, 308, and 325 with reference to SEQ ID NO: 107 that includes T308S, G306S, G305M, and P325S. A modified XIAP can include one or more amino acid substitutions including each of 305, 306, 308, and 325 with reference to SEQ ID NO: 107 that includes T308D, G306S, G305M, and P325S. A modified XIAP can include an amino acid substitution at position 305 of SEQ ID NO: 107. A modified XIAP can include an amino acid substitution at position 305 of SEQ ID NO: 107 that is G305M. A modified XIAP can include an amino acid substitution at position 306 of SEQ ID NO: 107. A modified XIAP can include an amino acid substitution at position 306 of SEQ ID NO: 107 that is G306S. A modified XIAP can include an amino acid substitution at position 308 of SEQ ID NO: 107. A modified XIAP can include an amino acid substitution at position 308 of SEQ ID NO: 107 that is T308S or T308D. A modified XIAP can include an amino acid substitution at position 308 of SEQ ID NO: 107 that is T308S. A modified XIAP can include an amino acid substitution at position 308 of SEQ ID NO: 107 that is T308D. A modified XIAP can include an amino acid substitution at position 325 of SEQ ID NO: 107. A modified XIAP can include an amino acid substitution at position 325 of SEQ ID NO: 107 that is P325S.

### Chimeric Proteins

In some aspects, the present disclosure provides chimeric proteins including a polypeptide of interest fused to the modified ER-LBD. Polypeptides of interest can include a cell death inducing domain, wherein the chimeric protein is configured upon contact with a ligand of the ligand binding domain to generate a cell-death inducing signal in a cell in which the polypeptide is expressed.

A polypeptide of interest can include a pro-apoptotic factor, such as a pro-apoptotic transcription factor. A polypeptide of interest can include a pro cell survival factor and/or an inhibitor of a pro cell survival factor. A polypeptide of interest can include polypeptide monomers that are generally inactive in monomeric form but active upon oligomerization.

The modified ER-LBD can be capable of inducing oligomerization and/or nuclear localization upon binding to a non-endogenous ligand. Thus, fusion of a modified ER-LBD to a polypeptide of interest may allow for control of cellular localization and/or oligomerization of the polypeptide of interest.

In some embodiments, the polypeptide of interest may be fused to the modified ER-LBD directly, or indirectly, e.g., via a linker. One or more linkers can be used between various domains of chimeric proteins, such as between an ER-LBD and a polypeptide of interest. For example, a polypeptide linker can include an amino acid sequence such as one or more of: GGGGSGGGGSGGGGSVDGF (SEQ ID NO: 4) and ASGGGGSAS (SEQ ID NO: 5).

In some embodiments, the polypeptide of interest includes at least one nucleic acid binding domain. In some embodiments, the nucleic acid binding domain is a zinc-finger domain. In some embodiments, the chimeric protein includes a transcription modulator, such as a transcription activator or a transcription repressor. Inclusion of a nucleic acid binding domain may allow for targeted nucleic acid binding by the chimeric protein that is inducible by a non-endogenous ligand (e.g., 4-OHT or endoxifen).

In some aspects, the nucleic acid binding domain comprises a DNA binding zinc finger protein domain (ZF protein domain). In some aspects, the ZF protein domain is modular in design and is composed of zinc finger arrays (ZFA). In some aspects, the transcriptional effector domain is selected from a Herpes Simplex Virus Protein 16 (VP16) activation domain; an activation domain comprising four tandem copies of VP16, a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); a histone acetyltransferase (HAT) core domain of the human E1A-associated protein p300 (p300 HAT core activation domain); a Krüppel associated box (KRAB) repression domain; a Repressor Element Silencing Transcription Factor (REST) repression domain; a WRPW motif (SEQ ID NO:156) of the hairy-related basic helix-loop-helix repressor proteins, the motif is known as a WRPW (SEQ ID NO:156) repression domain; a DNA (cytosine-5)-methyltransferase 3B (DNMT3B) repression domain; and an HP1 alpha chromoshadow repression domain.

In some embodiments, the ZF protein domain is modular in design and is composed of zinc finger arrays (ZFA). A zinc finger array comprises multiple zinc finger protein motifs that are linked together. Each zinc finger motif binds to a different nucleic acid motif. This results in a ZFA with specificity to any desired nucleic acid sequence. The ZF motifs can be directly adjacent to each other, or separated by a flexible linker sequence. In some embodiments, a ZFA is an array, string, or chain of ZF motifs arranged in tandem. A ZFA can have 1, 2, 3, 4, 5, 6, 7, 8, 9, 10, 11, 12, 13, 14, or 15 zinc finger motifs. The ZFA can have from 1-10, 1-15, 1-2, 1-3, 1-4, 1-5, 1-6, 1-7, 1-8, 1-9, 2-3, 2-4, 2-5, 2-6, 2-7, 2-8, 2-9, 2-10, 3-4, 3-5 3-6, 3-7, 3-8, 3-9, 3-10, 4-5, 4-6, 4-7, 4-8, 4-9, 4-10, 5-6, 5-7, 5-8, 5-9, 5-10, or 5-15 zinc finger motifs.

The ZF protein domain can have 1, 2, 3, 4, 5, 6, 7, 8, 9, 10, 11, 12, 13, 14, 15, or more ZFAs. The ZF domain can have from 1-10, 1-15, 1-2, 1-3, 1-4, 1-5, 1-6, 1-7, 1-8, 1-9, 2-3, 2-4, 2-5, 2-6, 2-7, 2-8, 2-9, 2-10, 3-4, 3-5 3-6, 3-7, 3-8, 3-9, 3-10, 4-5, 4-6, 4-7, 4-8, 4-9, 4-10, 5-6, 5-7, 5-8, 5-9, 5-10, or 5-15 ZFAs. In some embodiments, the ZF protein domain comprises one to ten ZFA(s). In some embodiments, the ZF protein domain comprises at least one ZFA. In some embodiments, the ZF protein domain comprises at least two ZFAs. In some embodiments, the ZF protein domain comprises at least three ZFAs. In some embodiments, the ZF protein domain comprises at least four ZFAs. In some embodiments, the ZF protein domain comprises at least five ZFAs. In some embodiments, the ZF protein domain comprises at least ten ZFAs.

An exemplary ZF protein domain is shown in the sequence SRPGERPFQCRICMRNFSRRHGLDRHTRTHTGEKPFQCRICMRNFSDHSSLKRHLR THTGSQKPFQCRICMRNFSVRHNLTRHLRTHTGEKPFQCRICMRNFSDHSNLSRHL KTHTGSQKPFQCRICMRNFSQRSSLVRHLRTHTGEKPFQCRICMRNFSESGHLKRH LRTHLRGS (SEQ ID NO: 6). In some embodiments, a ZF protein domain is a ZF5-7 DNA binding domain. An exemplary ZF5-7 DNA binding domain is shown in the sequence

(SEQ ID NO: 62)
MSRPGERPFQCRICMRNFSNMSNLTRHTRTHTGEKPFQCRICMRNFSDR
SVLRRHLRTHTGSQKPFQCRICMRNFSDPSNLARHTRTHTGEKPFQCRI
CMRNFSDRSSLRRHLRTHTGSQKPFQCRICMRNFSQSGTLHRHTRTHTG
EKPFQCRICMRNFSQRPNLTRHLRTHLRGS.

In some embodiments, the chimeric protein is a chimeric transcription factor and includes, in addition to the modified ER-LBD, a nucleic acid binding domain and a transcriptional modulator domain. In some aspects, the nucleic acid binding domain and the transcriptional modulator domain are part of the same naturally occurring protein. In some aspects, the nucleic acid binding domain and the transcriptional modulator domain are heterologous and do not exist naturally within the same protein.

“Transcriptional modulator domain” and “transcriptional effector domain” as used herein refers to a polypeptide domain that, when targeted to a promoter region of a gene (e.g., by a nucleic acid binding domain that specifically binds to a promoter of interest), is capable of modulating the transcription of the gene. In some aspects, the transcriptional modulator domain comprises a transcriptional repressor. In some aspects, the transcriptional repressor comprises a transcriptional repressor domain selected from a Krüppel associated box (KRAB) repression domain; a Repressor Element Silencing Transcription Factor (REST) repression domain; a WRPW motif (SEQ ID NO:156) of the hairy-related basic helix-loop-helix repressor proteins, the motif is known as a WRPW (SEQ ID NO: 156) repression domain; a DNA (cytosine-5)-methyltransferase 3B (DNMT3B) repression domain; and an HP1 alpha chromoshadow repression domain.

In some aspects, the transcriptional modulator domain comprises a transcriptional activator. In some aspects, the transcriptional activator comprises a transcriptional activator domain selected from a Herpes Simplex Virus Protein 16 (VP16) activation domain; an activation domain comprising four tandem copies of VP16; a VP64 activation domain; a p65 activation domain of NFκB (i.e., p65); an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase (HAT) core domain of the human E1A-associated protein p300 (p300 HAT core activation domain). In some aspects, the transcriptional modulator domain comprises a p65 transcriptional activator. In some aspects, a p65 transcriptional activator comprises the amino acid sequence

(SEQ ID NO: 64)
DEFPTMVFPSGQISQASALAPAPPQVLPQAPAPAPAPAMVSALAQAPAP
VPVLAPGPPQAVAPPAPKPTQAGEGTLSEALLQLQFDDEDLGALLGNST
DPAVFTDLASVDNSEFQQLLNQGIPVAPHTTEPMLMEYPEAITRLVTGA
QRPPDPAPAPLGAPGLPNGLLSGDEDFSSIADMDFSALLSQISS.

### Modified Estrogen Receptor Ligand Binding Domains (ER-LBD)

The present disclosure provides a modified estrogen receptor ligand binding domain (ER-LBD) comprising an amino acid sequence corresponding to a hormone binding domain of a reference human estrogen receptor sequence, SEQ ID NO: 1 (human Estrogen Receptor, UniProt ID No: P03372), comprising amino acid substitutions G400V, M543A, and L544A or amino acid substitutions G400V, M543A, L544A, and V595A, and comprising one or more additional amino acid substitutions to ligand binding residues selected from: positions 343-354, positions 380-392, positions 404-463, and positions 517-540, and position 547. It is to be understood that such amino acid substitutions are with reference to SEQ ID NO: 1. In some aspects, the one or more amino acid substitutions result in: (a) greater sensitivity to a non-endogenous ligand as compared to an endogenous ligand, (b) greater sensitivity to a non-endogenous ligand as compared to an ER-LBD of SEQ ID NO: 2, SEQ ID NO: 3, SEQ ID NO: 3A, or SEQ ID NO: 3B, and/or (c) greater selectivity to a non-endogenous ligand as compared to an ER-LBD of SEQ ID NO: 2, SEQ ID NO: 3, SEQ ID NO: 3A, or SEQ ID NO: 3B.

In some embodiments, the one or more additional amino acid substitutions results in greater sensitivity to a non-endogenous ligand as compared to an ER-LBD of SEQ ID NO: 2. In some embodiments, the one or more additional amino acid substitutions results in greater sensitivity to a non-endogenous ligand as compared to an ER-LBD of SEQ ID NO: 3. In some embodiments, the one or more additional amino acid substitutions results in greater sensitivity to a non-endogenous ligand as compared to an ER-LBD of SEQ ID NO: 3A. In some embodiments, the one or more additional amino acid substitutions results in greater sensitivity to a non-endogenous ligand as compared to an ER-LBD of SEQ ID NO: 3B. In some embodiments, the one or more additional amino acid substitutions results in greater selectivity to a non-endogenous ligand as compared to an ER-LBD of SEQ ID NO: 2. In some embodiments, the one or more additional amino acid substitutions results in greater selectivity to a non-endogenous ligand as compared to an ER-LBD of SEQ ID NO: 3. In some embodiments, the one or more additional amino acid substitutions results in greater selectivity to a non-endogenous ligand as compared to an ER-LBD of SEQ ID NO: 3A. In some embodiments, the one or more additional amino acid substitutions results in greater selectivity to a non-endogenous ligand as compared to an ER-LBD of SEQ ID NO: 3B.

In some embodiments, the modified ER-LBD may further comprise yet other modifications, e.g., amino acid substitutions, deletions, and/or insertions (with reference to SEQ ID NO: 1). Such yet other modifications can be within or outside of positions 343-354, positions 380-392, positions 404-463, positions 517-540, and/or position 547, with reference to SEQ ID NO: 1. Such yet other modifications can be within or outside of outside of positions 283-594, with reference to SEQ ID NO: 1.

“Ligand binding residues” refers to residues located at the ligand binding pocket of estrogen receptor (ER) or an ER-ligand binding domain, and includes the pocket for binding to an endogenous ligand (e.g., estradiol) and the pocket for binding to a non-endogenous ligand such as 4-OHT. In some embodiments, the hormone binding domain of a reference human estrogen receptor sequence corresponds to positions 282-595 of human estrogen receptor (SEQ ID NO: 1). It is to be understood that the hormone binding domain does not necessarily require all of amino acid residues 282-595 of SEQ ID NO: 1. By way of example only, it is to be understood that positions 283-594 of SEQ ID NO: 1, or other functional truncations or fragments thereof, may function as the hormone binding domain.

Residues within positions 343-354, positions 380-392 and positions 404-463 corresponding to SEQ ID NO: 1 are involved in binding to both endogenous and non-endogenous ligands. Residues within positions 517-547 (e.g., residues 517-40 and residue 547) corresponding to SEQ ID NO: 1 are located within a helix referred to as helix 12 and are involved in endogenous ligand binding.

Greater sensitivity to a non-endogenous ligand as compared to sensitivity to a non-endogenous ligand means that the modified ER-LBD binds to a non-endogenous ligand (e.g., endoxifen) with a higher affinity as compared to the affinity of its binding to an endogenous ligand (e.g., estradiol).

Greater sensitivity to a non-endogenous ligand as compared to sensitivity an ER-LBD not including the one or more amino acid substitutions (e.g., an ER-LBD comprising the amino acid sequence of SEQ ID NO: 2 or SEQ ID NO: 3) means that the modified ER-LBD binds to a non-endogenous ligand (e.g., endoxifen) with a higher affinity as compared to the affinity of binding of ER-LBD not including the one or more additional amino acid substitutions to the non-endogenous ligand. In some embodiments, the greater sensitivity is at least a 1.5-fold, at least a 2-fold, at least a 3-fold, at least a 4-fold, or at least a 5-fold improvement in binding affinity to a non-endogenous ligand, as compared to binding of an ER-LBD not including the one or more additional amino acid substitutions. In some embodiments, greater sensitivity is demonstrated by greater transcriptional modulation (e.g., greater transcriptional activation or greater transcriptional repression) of a chimeric transcription factor including a modified ER-LBD, as compared to a chimeric transcription factor including an ER-LBD that lacks the one or more additional amino acid substitutions. In some embodiments, in a transfection of transduction assay, a chimeric transcription factor including a modified ER-LBD is capable of inducing at least 10%, at least 15%, at least 20%, at least 25%, at least 30%, or at least 35% greater expression of a reporter under control of a chimeric transcription factor-responsive promoter in response to a non-endogenous ligand (e.g., 4-OHT) (as measured by % of cells positive for the reporter, or as measured by geometric mean fluorescent intensity) as compared to the expression of the reporter under the same conditions but with an ER-LBD that lacks the one or more additional amino acid substitutions.

Greater selectivity to a non-endogenous ligand refers to preferential binding to a non-endogenous ligand (e.g., 4-OHT or endoxifen) as compared to an endogenous ligand (e.g., estradiol). Selectivity may be measured using a selectivity coefficient, which is the equilibrium constant for the reaction of displacement by one ligand (e.g., a non-endogenous ligand) of another ligand (e.g., an endogenous ligand) in a complex with the substrate (e.g., a modified ER-LBD). The greater the selectivity coefficient, the more a competing ligand (e.g., an endogenous ligand) will displace the initial ligand (e.g., a non-endogenous ligand) from the complex formed with the substrate (e.g., a modified ER-LBD). In some embodiments, greater selectivity is demonstrated by improved transcriptional modulation of a chimeric transcription factor in the presence of a non-endogenous ligand as compared to transcriptional modulation in the presence of an endogenous ligand. In some embodiments, in a transfection of transduction assay, a chimeric transcription factor including a modified ER-LBD is capable of inducing at least 10%, at least 15%, at least 20%, at least 25%, at least 30%, or at least 35% greater expression of a reporter under control of a chimeric transcription factor-responsive promoter in response to a non-endogenous ligand (e.g., 4-OHT) (as measured by % of cells positive for the reporter, or as measured by geometric mean fluorescent intensity) as compared to the expression of the reporter under the same conditions but in response to an endogenous ligand (e.g., estradiol).

In some aspects, the one or more amino acid substitutions to ligand binding residues include one or more amino acid substitutions within helix 12. Helix 12 of an ER-LBD includes residue positions 533-547 of SEQ ID NO: 1. In some embodiments, the one or more amino acid substitutions within helix 12 are at one or more positions selected from 538, 536, 539, 540, 547, 534, 533, and 537.

“Non-endogenous ligand” may refer to, for example, a synthetic estrogen receptor binding ligand that is not naturally expressed by an organism that expresses an estrogen receptor. Non-endogenous estrogen receptor binding ligands include, without limitation, tamoxifen and metabolites thereof, such as 4-hydroxytamoxifen, N-desmethyltamoxifen, tamoxifen-N-oxide, and endoxifen.

The one or more additional amino acid substitutions may be at one or more positions of SEQ ID NO: 1 selected from 343, 344, 345, 346, 347, 348, 349, 350, 351, 352, 354, 380, 384, 386, 387, 388, 389, 391, 392, 404, 407, 409, 413, 414, 417, 418, 420, 421, 422, 424, 428, 463, 517, 521, 522, 524, 525, 526, 527, 528, 533, 534, 536, 537, 538, 539, 540, and 547. In some embodiments, the one or more additional amino acid substitutions include substitutions at one of the above-listed positions, two of the above-listed positions, three of the above-listed positions, four of the above-listed positions, or five of the above-listed positions.

In some aspects, the one or more additional amino acids substitutions are selected from one or more of the substitutions listed in Table 1.

TABLE 1
Amino Acid Substitutions

| | | | | | |
|---|---|---|---|---|---|
| M343I | L349I | L384M | N413D | L428F | L525S |
| M343L | L349M | L384V | Q414E | M517A | Y526L |
| M343V | L349V | L384F | C417S | G521A | S527N |
| M343F | L349F | I386V | V418I | G521I | M528I |
| G344M | A350L | L387I | V418L | G521L | M528V |
| L345S | A350M | L387M | V418M | G521M | M528F |
| L346I | A350V | L387V | V418F | G521V | V533F |
| L346M | A350F | L387F | G420I | G521F | V533W |
| L346V | A350I | M388I | G420M | M522I | V534R |
| L346F | D351I | M388L | G420V | M522L | V534Q |
| T347I | D351L | M388F | G420F | M522V | L536F |
| T347L | D351M | I389M | M421I | H524A | L536M |
| T347M | D351N | L391I | M421L | H524I | L536Y |
| T347N | D351V | L391M | M421V | H524L | Y537E |
| T347R | D351E | L391V | M421F | H524V | D538K |
| T347V | D351F | L391F | V422I | H524F | L539R |
| T347D | D351Q | V392M | I424L | L525I | L539A |
| T347E | R352K | F404I | I424M | L525M | L540A |
| T347F | L354I | F404L | I424V | L525N | L540F |
| T347K | L354M | F404M | I424F | L525T | H547A |
| T347Q | L354V | F404V | L428I | L525V | S463P |
| T347S | L354F | N407D | L428M | L525F | |
| N348K | L384I | L409V | L428V | L525Q | |

In some aspects, the one or more additional mutations comprise at least two mutations, at least three mutations, at least four mutations, at least five mutations, at least six mutations, at least seven mutations, or at least eight mutations. In some aspects, the one or more additional mutations comprise two to ten mutations, two to nine mutations, two to eight mutations, two to seven mutations, two to six mutations, two to five mutations, two to four mutations, two to three mutations, three to ten mutations, three to nine mutations, three to eight mutations, three to seven mutations, three to six mutations, three to five mutations, three to four mutations, four to ten mutations, four to nine mutations, four to eight mutations, four to seven mutations, four to six mutations, four to five mutations, five to ten mutations, five to nine mutations, five to eight mutations, five to seven mutations, five to six mutations, six to ten mutations, six to nine mutations, six to eight mutations, six to seven mutations, seven to ten mutations, seven to nine mutations, seven to eight mutations, eight to ten mutations, eight to nine mutations, or nine to ten mutations.

In some aspects, the one or more additional mutations comprise at least two mutations that are selected from the mutations listed in Table 2.

TABLE 2
Combination Amino Acid Substitutions

| | |
|---|---|
| L345S_N348K | L384M_L391F |
| L384M_I389M | L354I_L384M |
| M421I_V392M | L354I_L384M_L391F |
| L354I_L391F | L354I_L384M_L391F_V418I |
| L354I_L387M | M343I_M388I_G521I_F404L |
| L387M_L391F | H524V_T347R_D351Q_L525N |
| L384M_L387M | L354I_L384M_L391F_V418I |
| Q414E_S463P_H524L | L354I_L384M_L391V_S463P |
| L384M_L391V_N413D_H524F | L384M_L409V_N413D_S463P_H524L |
| L391V_N413D_Q414E_S463P_H524F | L391V_Q414E_M421L_S463P_H524F |
| L354I_L409V_N413D_M421L_H524L | L354I_L409V_M421L_S463P_H524L |
| L384M_L391V_N413D_M421L_S463P_H524L | L409V_N413D_Q414E_M421L_S463P_H524L |
| L354I_L391V_L409V_N413D_Q414E_H524L | L354I_L384M_L409V_N413D_M421L_S463P_H524F |
| L354I_L391V_N413D_M421L_S463P_M517A_H524L | L354I_L391V_N413D_Q414E_M421L_M517A_H524F |
| L384M_L391V_L409V_N413D_M421L_S463P_M517A_H524F | |

In some embodiments, the one or more additional amino acid substitutions include an L391V substitution and an N413D mutation. In some embodiments, the one or more additional amino acid substitutions include an L391V substitution, an N413D mutation, and an H524 substitution. In some embodiments, the one or more additional amino acid substitutions include an L391V substitution, an N413D mutation, an H524 substitution, and an M421L substitution. In some embodiments, the one or more additional amino acid substitutions include an L391V substitution, an N413D mutation, an H524 substitution, and an S463P substitution. In some embodiments, the one or more additional amino acid substitutions include an L391V substitution, an N413D mutation, an H524 substitution, and an Q414E substitution. In some embodiments, the one or more additional amino acid substitutions include an L391V substitution, an N413D mutation, an H524 substitution, and an L354I substitution. The H524 substitution may be a H524F or a H524L substitution.

In particular embodiments, the modified ER-LBD includes (a) a G400V amino acid substitution, an M543A amino acid substitution, an L544A amino acid substitution, and optionally a V595A amino acid substitution; and (b) additional amino acid substitutions, wherein the additional amino acid substitutions include: (i) an L384M substitution, an L391V substitution, a N413D substitution, an M421L substitution, a S463P substitution, and a H524L substitution, (ii) an L391V substitution, a N413D substitution, a Q414E substitution, a S463P substitution, and a H524F substitution, (iii) an L354I substitution, a L391V substitution, a N413D substitution, a Q414E substitution, a M421L substitution, a M517A substitution, and a H524F substitution, or (iv) an L354I substitution, a L391V substitution, a L409V substitution, a N413D substitution, a Q414E substitution, and a H524L substitution.

In some embodiments, the modified ER-LBD comprising the additional amino acid substitutions L384M, L391V, N413D, M421L, S463P, and H524L comprises an amino acid sequence at least 80%, 85%, 90%, 95%, 97.5%, 98%, 99%, or 100% identical to SEQ ID NO: 41 or 116.

In some embodiments, the modified ER-LBD comprising the additional amino acid substitutions L391V, N413D, Q414E, S463P, and H524F comprises an amino acid sequence at least 80%, 85%, 90%, 95%, 97.5%, 98%, 99%, or 100% identical to SEQ ID NO: 45.

In some embodiments, the modified ER-LBD comprising the additional amino acid substitutions L354I, L391V, N413D, Q414E, M421L, M517A, and H524F comprises an amino acid sequence at least 80%, 85%, 90%, 95%, 97.5%, 98%, 99%, or 100% identical to SEQ ID NO: 50.

In some embodiments, the modified ER-LBD comprising the additional amino acid substitutions L354I, L391V, L409V, N413D, Q414E, and H524L comprises an amino acid sequence at least 80%, 85%, 90%, 95%, 97.5%, 98%, 99%, or 100% identical to SEQ ID NO: 52.

In some embodiments, the one or more additional amino acid substitutions include an N413D mutation, an H524 substitution, and an S463P substitution. The H524 substitution may be a H524F or a H524L substitution. In some embodiments, the one or more additional amino acid substitutions include an N413D mutation, an H524L substitution, and an S463P substitution.

In some embodiments, the modified ER-LBD comprises the additional amino acid substitutions L391V, L409V, Q414E, N413D, S463P, M517A, and H524L. In some embodiments, the modified ER-LBD comprising the additional amino acid substitutions L391V, L409V, Q414E, N413D, S463P, M517A, and H524L comprises an amino acid sequence at least 80%, 85%, 90%, 95%, 97.5%, 98%, 99%, or 100% identical to SEQ ID NO: 90 or 103.

In some embodiments, the modified ER-LBD comprises the additional amino acid substitutions L409V, N413D, S463P, M421L, L384M, and H524L. In some embodiments, the modified ER-LBD comprising the additional amino acid substitutions L409V, N413D, S463P, M421L, L384M, and H524L comprises an amino acid sequence at least 80%, 85%, 90%, 95%, 97.5%, 98%, 99%, or 100% identical to SEQ ID NO: 91 or 104.

In some embodiments, the modified ER-LBD comprising the additional amino acid substitutions L391V, L409V, N413D, S463P, M517A, M421L, L354I, and H524L comprises an amino acid sequence at least 80%, 85%, 90%, 95%, 97.5%, 98%, 99%, or 100% identical to SEQ ID NO: 92 or 105.

In some embodiments, the modified ER-LBD comprising the additional amino acid substitutions L391V, Q414E, N413D, S463P, M421L, L354I, L384M, and H524L comprises an amino acid sequence at least 80%, 85%, 90%, 95%, 97.5%, 98%, 99%, or 100% identical to SEQ ID NO: 93 or 106.

In some embodiments, the modified ER-LBD comprising the additional amino acid substitutions L391V, LA09V, N413D, S463P, M517A, M421L, and H524L comprises an amino acid sequence at least 80%, 85%, 90%, 95%, 97.5%, 98%, 99%, or 100% identical to SEQ ID NO: 94 or 107.

In some embodiments, the modified ER-LBD comprising the additional amino acid substitutions L391V, L409V, Q414E, N413D, S463P, L354I, and H524L comprises an amino acid sequence at least 80%, 85%, 90%, 95%, 97.5%, 98%, 99%, or 100% identical to SEQ ID NO: 95 or 108.

In some embodiments, the modified ER-LBD comprising the additional amino acid substitutions L391V, L409V, N413D, S463P, M421L, L354I, L384M, and H524L comprises an amino acid sequence at least 80%, 85%, 90%, 95%, 97.5%, 98%, 99%, or 100% identical to SEQ ID NO: 96 or 109.

In some embodiments, the modified ER-LBD comprising the additional amino acid substitutions L391V, Q414E, N413D, S463P, M517A, M421L, L354I, and H524L comprises an amino acid sequence at least 80%, 85%, 90%, 95%, 97.5%, 98%, 99%, or 100% identical to SEQ ID NO: 97 or 110.

In some embodiments, the modified ER-LBD comprising the additional amino acid substitutions L391V, N413D, S463P, M517A, L384M, and H524L comprises an amino acid sequence at least 80%, 85%, 90%, 95%, 97.5%, 98%, 99%, or 100% identical to SEQ ID NO: 98 or 111.

In some embodiments, the modified ER-LBD comprising the additional amino acid substitutions L391V, L409V, N413D, S463P, M517A, and H524L comprises an amino acid sequence at least 80%, 85%, 90%, 95%, 97.5%, 98%, 99%, or 100% identical to SEQ ID NO: 99 or 112.

In some embodiments, the modified ER-LBD comprising the additional amino acid substitutions N413D, S463P, L354I, L384M, and H524L comprises an amino acid sequence at least 80%, 85%, 90%, 95%, 97.5%, 98%, 99%, or 100% identical to SEQ ID NO: 100 or 113.

In some embodiments, the modified ER-LBD comprising the additional amino acid substitutions N413D, S463P, M421L, L354I, and H524L comprises an amino acid sequence at least 80%, 85%, 90%, 95%, 97.5%, 98%, 99%, or 100% identical to SEQ ID NO: 101 or 114.

### Molecular Switches

Also provided herein are molecular switches for a cell-death inducing signal in a cell. A molecular switch may include (a) any of the inducible cell death systems described herein (including any of the isolated polynucleotides, heterologous constructs, plasmids, vectors, or cells described herein), and (b) a non-endogenous ligand that binds to the modified ER-LBD of the chimeric protein.

Upon binding of the non-endogenous ligand to the modified ER-LBD, the chimeric protein generally generates a cell-death inducing signal.

A molecular switch may include (a) an inducible cell-death system including a polypeptide, where the polypeptide includes a ligand binding domain and a cell death inducing domain, where the polypeptide is configured upon contact with a ligand of the ligand binding domain to generate a cell-death inducing signal in a cell in which the polypeptide is expressed, and where the ligand binding domain includes a modified ER-LBD (such as any of the modified ER-LBDs described herein); and (b) a non-endogenous ligand, where binding of the non-endogenous ligand to the modified ER-LBD generates the cell-death inducing signal in the cell.

A molecular switch may include (a) an inducible cell-death system including a first polypeptide monomer and a second polypeptide monomer, where the first polypeptide monomer and the second polypeptide monomer each include a ligand binding domain and a cell death inducing domain, wherein the first polypeptide monomer and the second polypeptide monomer are configured to oligomerize with each other upon contact with a ligand of the ligand binding domain, thereby generating the cell-death inducing signal in a cell in which the first polypeptide monomer and the second polypeptide monomer are expressed, and where the ligand binding domain includes a modified ER-LBD (such as any of the modified ER-LBDs described herein); and (b) a non-endogenous ligand, where binding of the non-endogenous ligand to the modified ER-LBD induces oligomerization of the first and the second polypeptide monomers, thereby generating the cell-death inducing signal in the cell.

Also provided herein are inducible cell death systems that include a cell death-inducing domain that is a transcription factor including a nucleic acid-binding domain and a transcriptional effector domain, wherein the transcription factor is configured to generate a cell-death inducing signal by inducing expression of a gene of interest. Transcription factors configured to generate a cell-death inducing signal may include (a) a chimeric transcription factor that includes a modified ER-LBD and is capable of binding to a chimeric transcription factor-responsive promoter (CTF-responsive promoter) operably linked to a gene of interest, and (b) a non-endogenous ligand that binds to the modified ER-LBD of the chimeric protein. Upon binding of the non-endogenous ligand to the modified ER-LBD, the chimeric protein may modulate transcription of a gene of interest.

In some embodiments, the gene of interest encodes a polypeptide selected from: a caspase domain or derivative or functional fragment thereof, optionally wherein the caspase is selected from any one of caspases 1-11, such as caspase 3, caspase 6, caspase 7, caspase 8, caspase 9, or derivatives or functional fragments thereof, respectively, Diphtheria toxin fragment A (DTA), Bax, Bak, Bok, Bad, Bcl-Xs, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B1, or Purine nucleoside phosphorylase.

In some embodiments, the non-endogenous ligand is selected from 4-hydroxytamoxifen (4-OHT), N-desmethyltamoxifen, tamoxifen-N-oxide, and endoxifen.

In particular embodiments, the non-endogenous ligand is 4-hydroxytamoxifen (4-OHT, also referred to as afimoxifene). In particular embodiments, the non-endogenous ligand includes a tamoxifen metabolite, such as 4-OHT, endoxifen, or a combination of 4-OHT and endoxifen. In particular embodiments, the non-endogenous ligand is endoxifen. In particular embodiments, the molecular switch is capable of generating the cell-death inducing signal at a concentration of 0.25 nM Endoxifen or less and/or at a concentration of 0.04 nM 4-OHT or less. In particular embodiments, the molecular switch is capable of generating the cell-death inducing signal at a concentration of 2.5 nM Endoxifen or less and/or at a concentration of 0.4 nM 4-OHT or less. In particular embodiments, the molecular switch is capable of generating the cell-death inducing signal at a concentration of at least 0.001 pM of 4-OHT. In particular embodiments, the molecular switch is capable of generating the cell-death inducing signal at a concentration of at least 0.01 pM of 4-OHT.

In particular embodiments, the first polypeptide monomer and the second polypeptide monomer are capable of oligomerization and/or generating the cell-death inducing signal at a concentration of 0.25 nM Endoxifen or less and/or at a concentration of 0.04 nM 4-OHT or less. In particular embodiments, the first polypeptide monomer and the second polypeptide monomer are capable of oligomerization and/or generating the cell-death inducing signal at a concentration of 2.5 nM Endoxifen or less and/or at a concentration of 0.4 nM 4-OHT or less. In particular embodiments, the first polypeptide monomer and the second polypeptide monomer are capable of oligomerization and/or generating the cell-death inducing signal at a concentration of at least 0.001 pM of 4-OHT. In particular embodiments, the first polypeptide monomer and the second polypeptide monomer are capable of oligomerization and/or generating the cell-death inducing signal at a concentration of at least 0.01 pM of 4-OHT.

### Isolated Polynucleotides and Heterologous Constructs

Also provided herein are isolated polynucleotides and heterologous constructs encoding an inducible a modified ER-LBD or chimeric protein (e.g., any of the polypeptides, the first polypeptide monomers, and/or the second polypeptide monomers of the inducible cell death systems herein) as described herein. In some aspects the present disclosure provides an isolated polynucleotide comprising a nucleotide sequence encoding a modified ER-LBD or chimeric protein as described herein. In some aspects, the present disclosure provides a heterologous construct comprising a promoter operatively linked to the polynucleotide encoding the modified ER-LBD or chimeric protein.

In some aspects, the present disclosure further provides isolated polynucleotides and/or heterologous constructs including a target gene expression cassette.

“Isolated” nucleic acid molecule or polynucleotide refers to a nucleic acid molecule, such as DNA or RNA, which has been removed from its native environment. For example, a polynucleotide encoding a modified ER-LBD or chimeric protein contained in a heterologous construct is considered isolated. Further examples of an isolated polynucleotide include recombinant polynucleotides maintained in heterologous host cells or purified (partially or substantially) polynucleotides in solution. An isolated polynucleotide also includes a polynucleotide contained in cells that ordinarily contain the polynucleotide, but the polynucleotide is present extrachromosomally or at a chromosomal location that is different from its natural chromosomal location.

Isolated polynucleotides include, but are not limited to a cDNA polynucleotide, an RNA polynucleotide, an RNAi oligonucleotide (e.g., siRNAs, miRNAs, antisense oligonucleotides, shRNAs, etc.), an mRNA polynucleotide, a circular plasmid, a linear DNA fragment, a vector, a minicircle, a ssDNA, a bacterial artificial chromosome (BAC), and yeast artificial chromosome (YAC), and an oligonucleotide.

In some embodiments, the isolated polynucleotide is selected from: a DNA, a cDNA, an RNA, an mRNA, and a naked plasmid (linear or circular).

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs.

In some aspects, the chimeric protein encoded by the polynucleotide is a polypeptide of the inducible cell death systems described herein. In some aspects, the chimeric protein encoded by the polynucleotide is a first polypeptide monomer of the inducible cell death systems described herein. In some aspects, the chimeric protein encoded by the polynucleotide is a second polypeptide monomer of the inducible cell death systems described herein. In some aspects, the chimeric protein encoded by the polynucleotide is a first polypeptide monomer and a second polypeptide monomer of the inducible cell death systems described herein. In some aspects, the chimeric protein encoded by the polynucleotide is a chimeric transcription factor, and the polynucleotide further includes a target expression cassette including a gene of interest operably linked to a chimeric transcription factor-responsive (CTF-responsive) promoter. In some embodiments, the target expression cassette is present in the same heterologous construct as the chimeric protein. In some embodiments, the chimeric protein and the target expression cassette are present in separate heterologous constructs.

The term “expression cassette” refers to a polynucleotide generated recombinantly or synthetically, with a series of nucleic acid elements that permit transcription of a particular polynucleotide in a target cell. The expression cassette can be incorporated into a plasmid, chromosome, mitochondrial DNA, plastid DNA, virus, or nucleic acid fragment. Typically, the expression cassette portion of an expression vector includes, among other sequences, a nucleic acid sequence to be transcribed and a promoter. In some aspects, the present disclosure provides an expression cassette including a polynucleotide encoding a modified ER-LBD or a chimeric protein including a modified ER-LBD.

The isolated polynucleotides and heterologous constructs including a modified ER-LBD as described herein are engineered polynucleotides. An “engineered polynucleotide” is a polynucleotide that does not occur in nature. It should be understood, however, that while an engineered polynucleotide as a whole is not naturally-occurring, it may include nucleotide sequences that occur in nature. In some embodiments, an engineered polynucleotide comprises nucleotide sequences from different organisms (e.g., from different species). For example, in some embodiments, an engineered polynucleotide includes a murine nucleotide sequence, a bacterial nucleotide sequence, a human nucleotide sequence, and/or a viral nucleotide sequence. The term “engineered polynucleotide” includes recombinant nucleic acids and synthetic nucleic acids. A “recombinant polynucleotide” refers to a molecule that is constructed by joining nucleotide molecules and, in some embodiments, can replicate in a live cell. A “synthetic polynucleotide” refers to a molecule that is amplified or chemically, or by other means, synthesized. Synthetic polynucleotides include those that are chemically modified, or otherwise modified, but can base pair with naturally-occurring nucleotide molecules. Modifications include, but are not limited to, one or more modified internucleotide linkages and non-natural nucleic acids. Modifications are described in further detail in U.S. Pat. No. 6,673,611 and U.S. Application Publication 2004/0019001 and, each of which is incorporated by reference in their entirety. Modified internucleotide linkages can be a phosphorodithioate or phosphorothioate linkage. Non-natural nucleic acids can be a locked nucleic acid (LNA), a peptide nucleic acid (PNA), glycol nucleic acid (GNA), a phosphorodiamidate morpholino oligomer (PMO or “morpholino”), and threose nucleic acid (TNA). Non-natural nucleic acids are described in further detail in International Application WO 1998/039352, U.S. Application Pub. No. 2013/0156849, and U.S. Pat. Nos. 6,670,461; 5,539,082; 5,185,444, each herein incorporated by reference in their entirety. Recombinant polynucleotides and synthetic polynucleotides also include those molecules that result from the replication of either of the foregoing. Engineered polynucleotides of the present disclosure may be encoded by a single molecule (e.g., included in the same plasmid or other vector) or by multiple different molecules (e.g., multiple different independently-replicating molecules).

Engineered polynucleotides of the present disclosure may be produced using standard molecular biology methods (see, e.g., Green and Sambrook, Molecular Cloning, A Laboratory Manual, 2012, Cold Spring Harbor Press). In some embodiments, engineered nucleic acid constructs are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the ‘Y extension activity of a DNA polymerase and DNA ligase activity. The 5′ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies. In some embodiments, engineered nucleic acid constructs are produced using IN-FUSION® cloning (Clontech).

In some embodiments, the polynucleotides as described herein are included in a heterologous construct. The term “vector” or “expression vector” is synonymous with “heterologous construct” and refers to a polynucleotide that is used to introduce and direct the expression of one or more genes that are operably associated with the construct in a target cell. The term includes the construct as a self-replicating nucleic acid structure as well as the vector incorporated into the genome of a host cell into which it has been introduced. A heterologous construct as described herein includes an expression cassette. In some aspects, provided herein is a heterologous construct comprising an expression cassette that comprises a promoter operably linked to a polynucleotide that encodes a modified ER-LBD or a chimeric protein including a modified ER-LBD.

As used herein, a “promoter” refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, repressible, tissue-specific or any combination thereof. A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. Such a promoter can be referred to as “endogenous.” In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see, e.g., U.S. Pat. Nos. 4,683,202 and 5,928,906).

As used herein, an “inducible promoter” refers to a promoter characterized by regulating (e.g., initiating or activating) transcriptional activity when in the presence of, influenced by or contacted by a signal. The signal may be endogenous or a normally exogenous condition (e.g., light), compound (e.g., chemical or non-chemical compound) or protein (e.g., a chimeric transcription factor as described herein) that contacts an inducible promoter in such a way as to be active in regulating transcriptional activity from the inducible promoter. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter.

As used herein, a promoter is “responsive to” or “modulated by” a local tumor state (e.g., inflammation or hypoxia) or signal if in the presence of that state or signal, transcription from the promoter is activated, deactivated, increased, or decreased. In some embodiments, the promoter comprises a response element. A “response element” is a short sequence of DNA within a promoter region that binds specific molecules (e.g., transcription factors) that modulate (regulate) gene expression from the promoter. Response elements that may be used in accordance with the present disclosure include, without limitation, a phloretin-adjustable control element (PEACE), a zinc-finger DNA binding domain (DBD), an interferon-gamma-activated sequence (GAS) (Decker, T. et al. J Interferon Cytokine Res. 1997 March; 17 (3): 121-34, incorporated herein by reference), an interferon-stimulated response element (ISRE) (Han, K. J. et al. J Biol Chem. 2004 Apr. 9; 279 (15): 15652-61, incorporated herein by reference), a NF-kappaB response element (Wang, V. et al. Cell Reports. 2012; 2 (4): 824-839, incorporated herein by reference), and a STAT3 response element (Zhang, D. et al. J of Biol Chem. 1996; 271:9503-9509, incorporated herein by reference). Other response elements are encompassed herein. Response elements can also contain tandem repeats (e.g., consecutive repeats of the same nucleotide sequence encoding the response element) to generally increase sensitivity of the response element to its cognate binding molecule. Tandem repeats can be labeled 2×, 3×, 4×, 5×, etc. to denote the number of repeats present.

Non-limiting examples of responsive promoters (also referred to as “inducible promoters”) (e.g., TGF-beta responsive promoters) are listed in Table 3, which shows the design of the promoter and transcription factor, as well as the effect of the inducer molecule towards the transcription factor (TF) and transgene transcription (T) is shown (B, binding; D, dissociation; n.d., not determined) (A, activation; DA, deactivation; DR, derepression) (see Horner, M. & Weber, W. FEBS Letters 586 (2012) 20784-2096m, and references cited therein). Non-limiting examples of components of inducible promoters include those shown in Table 4.

TABLE 3

| | Promoter and | Transcription | Inducer | Response to inducer | |
|---|---|---|---|---|---|
| System | operator | factor (TF) | molecule | TF | T |
| Transcriptional activator-responsive promoters | | | | | |
| AIR | PAIR (OalcA- | AlcR | Acetaldehyde | n.d. | A |
| | PhCMVmin) | | | | |
| ART | PART (OARG- | ArgR-VP16 | l-Arginine | B | A |
| | PhCMVmin) | | | | |
| BIT | PBIT3 (OBirA3- | BIT (BirA-VP16) | Biotin | B | A |
| | PhCMVmin) | | | | |
| Cumate - activator | PCR5 (OCuO6- | cTA (CymR-VP16) | Cumate | D | DA |
| | PhCMVmin) | | | | |
| Cumate - reverse | PCR5 (OCuO6- | rcTA (rCymR-VP16) | Cumate | B | A |
| activator | PhCMVmin) | | | | |
| E-OFF | PETR (OETR- | ET (E-VP16) | Erythromycin | D | DA |
| | PhCMVmin) | | | | |
| NICE-OFF | PNIC (ONIC- | NT (HdnoR-VP16) | 6-Hydroxy- | D | DA |
| | PhCMVmin) | | nicotine | | |
| PEACE | PTtgR1 (OTtgR- | TtgA1 (TtgR-VP16) | Phloretin | D | DA |
| | PhCMVmin) | | | | |
| PIP-OFF | PPIR (OPIR- | PIT (PIP-VP16) | Pristinamycin I | D | DA |
| | Phsp70min) | | | | |
| QuoRex | PSCA (OscbR- | SCA (ScbR-VP16) | SCB1 | D | DA |
| | PhCMVmin)PSPA | | | | |
| | (OpapRI-PhCMVmin) | | | | |
| Redox | PROP (OROP- | REDOX (REX-VP16) | NADH | D | DA |
| | PhCMVmin) | | | | |
| TET-OFF | PhCMV*-1 (OtetO7- | tTA (TetR-VP16) | Tetracycline | D | DA |
| | PhCMVmin) | | | | |
| TET-ON | PhCMV*-1 (OtetO7- | rtTA (rTetR-VP16) | Doxycycline | B | A |
| | PhCMVmin) | | | | |
| TIGR | PCTA (OrheO- | CTA (RheA-VP16) | Heat | D | DA |
| | PhCMVmin) | | | | |
| TraR | O7x(tra box)- | p65-TraR | 3-Oxo-C8- | B | A |
| | PhCMVmin | | HSL | | |
| VAC-OFF | P1VanO2 (OVanO2- | VanA1 (VanR-VP16) | Vanillic acid | D | DA |
| | PhCMVmin) | | | | |
| Transcriptional repressor-responsive promoters | | | | | |
| Cumate - repressor | PCuO (PCMV5- | CymR | Cumate | D | DR |
| | OCuO) | | | | |
| E-ON | PETRON8 (PSV40- | E-KRAB | Erythromycin | D | DR |
| | OETR8) | | | | |
| NICE-ON | PNIC (PSV40- | NS (HdnoR-KRAB) | 6-Hydroxy- | D | DR |
| | ONIC8) | | nicotine | | |
| PIP-ON | PPIRON (PSV40- | PIT3 (PIP-KRAB) | Pristinamycin I | D | DR |
| | OPIR3) | | | | |
| Q-ON | PSCAON8 (PSV40- | SCS (ScbR-KRAB) | SCB1 | D | DR |
| | OscbR8) | | | | |
| TET-ON<comma> | OtetO-PHPRT | tTS-H4 (TetR-HDAC4) | Doxycycline | D | DR |
| repressor-based | | | | | |
| T-REX | PTetO (PhCMV- | TetR | Tetracycline | D | DR |
| | OtetO2) | | | | |
| UREX | PUREX8 (PSV40- | mUTS (KRAB-HucR) | Uric acid | D | DR |
| | OhucO8) | | | | |
| VAC-ON | PVanON8 (PhCMV- | VanA4 (VanR-KRAB) | Vanillic acid | D | DR |
| | OVanO8) | | | | |
| Hybrid promoters | | | | | |
| QuoRexPIP- | OscbR8-OPIR3- | SCAPIT3 | SCB1Pristinamycin I | DD | DADR |
| ON(NOT IF gate) | PhCMVmin | | | | |
| QuoRexE- | OscbR-OETR8- | SCAE-KRAB | SCB1Erythromycin | DD | DADR |
| ON(NOT IF gate) | PhCMVmin | | | | |
| TET-OFFE- | OtetO7-OETR8- | tTAE-KRAB | TetracyclineErythromycin | DD | DADR |
| ON(NOT IF gate) | PhCMVmin | | | | |
| TET-OFFPIP- | OtetO7-OPIR3- | tTAPIT3E-KRAB | TetracyclinePristinamycin | DDD | DADRDR |
| ONE-ON | OETR8-PhCMVmin | | IErythromycin | | |

TABLE 4

| Name | DNA SEQUENCE | Source |
|---|---|---|
| minimal promoter; | AGAGGGTATATAATGGAAGCTC | EU581860.1 |
| minP | GACTTCCAG (SEQ ID NO: 7) | (Promega) |
| NFkB response | GGGAATTTCCGGGGACTTTCCG | EU581860.1 |
| element protein | GGAATTTCCGGGGACTTTCCGG | (Promega) |
| promoter; 5x NFkB- | GAATTTCC (SEQ ID NO: 8) | |
| RE | | |
| CREB response | CACCAGACAGTGACGTCAGCTG | DQ904461.1 |
| element protein | CCAGATCCCATGGCCGTCATACT | (Promega) |
| promoter; 4x CRE | GTGACGTCTTTCAGACACCCCAT | |
| | TGACGTCAATGGGAGAA (SEQ ID | |
| | NO: 9) | |
| NFAT response | GGAGGAAAAACTGTTTCATACA | DQ904462.1 |
| element protein | GAAGGCGTGGAGGAAAAACTGT | (Promega) |
| promoter; 3x NFAT | TTCATACAGAAGGCGTGGAGGA | |
| binding sites | AAAACTGTTTCATACAGAAGGC | |
| | GT (SEQ ID NO: 10) | |
| SRF response element | AGGATGTCCATATTAGGACATCT | FJ773212.1 |
| protein promoter; 5x | AGGATGTCCATATTAGGACATCT | (Promega) |
| SRE | AGGATGTCCATATTAGGACATCT | |
| | AGGATGTCCATATTAGGACATCT | |
| | AGGATGTCCATATTAGGACATCT | |
| | (SEQ ID NO: 11) | |
| SRF response element | AGTATGTCCATATTAGGACATCT | FJ773213.1 |
| protein promoter 2; | ACCATGTCCATATTAGGACATCT | (Promega) |
| 5x SRF-RE | ACTATGTCCATATTAGGACATCT | |
| | TGTATGTCCATATTAGGACATCT | |
| | AAAATGTCCATATTAGGACATCT | |
| | (SEQ ID NO: 12) | |
| AP1 response element | TGAGTCAGTGACTCAGTGAGTC | JQ858516.1 |
| protein promoter; 6x | AGTGACTCAGTGAGTCAGTGAC | (Promega) |
| AP1-RE | TCAG (SEQ ID NO: 13) | |
| TCF-LEF response | AGATCAAAGGGTTTAAGATCAA | JX099537.1 |
| element promoter; 8x | AGGGCTTAAGATCAAAGGGTAT | (Promega) |
| TCF-LEF-RE | AAGATCAAAGGGCCTAAGATCA | |
| | AAGGGACTAAGATCAAAGGGTT | |
| | TAAGATCAAAGGGCTTAAGATC | |
| | AAAGGGCCTA (SEQ ID NO: 14) | |
| SBEx4 | GTCTAGACGTCTAGACGTCTAG | Addgene Cat No: |
| | ACGTCTAGAC (SEQ ID NO: 15) | 16495 |
| SMAD2/3- | CAGACACAGACACAGACACAGA | Jonk et al. (J Biol |
| CAGACA x4 | CA (SEQ ID NO: 16) | Chem. 1998 Aug. |
| | | 14; 273(33): |
| | | 21145-52. |
| STAT3 binding site | Ggatccggtactcgagatctgcgatctaagtaagctt | Addgene Sequencing |
| | ggcattccggtactgttggtaaagccac (SEQ ID | Result #211335 |
| | NO: 17) | |

Other non-limiting examples of promoters include the cytomegalovirus (CMV) promoter, the elongation factor 1-alpha (EF1a) promoter, the elongation factor (EFS) promoter, the MND promoter (a synthetic promoter that contains the U3 region of a modified MoMuLV LTR with myeloproliferative sarcoma virus enhancer), the phosphoglycerate kinase (PGK) promoter, the spleen focus-forming virus (SFFV) promoter, the simian virus 40 (SV40) promoter, and the ubiquitin C (UbC) promoter.

In some aspects, the present disclosure provides a heterologous construct comprising a promoter operatively linked to a polynucleotide encoding a modified ER-LBD or chimeric protein as described herein.

In some embodiments, the promoter operatively linked to a polynucleotide encoding a modified ER-LBD or chimeric protein is a constitutive promoter, an inducible promoter, or a synthetic promoter.

In some embodiments, the promoter operatively linked to a polynucleotide encoding a modified ER-LBD or chimeric protein is a constitutive promoter. Examples of constitutive promoters are shown in Table 5. In some embodiments, the constitutive promoter is selected from: CMV, EFS, SFFV, SV40, MND, PGK, UbC, hEF1aV1, hCAGG, hEFlaV2, hACTb, hcIF4A1, hGAPDH, hGRP78, hGRP94, hHSP70, hKINb, and hUBIb.

TABLE 5

| Name | DNA SEQUENCE |
|---|---|
| CMV | GTTGACATTGATTATTGACTAGTTATTAATAGTAATCAATTACGG |
| | GGTCATTAGTTCATAGCCCATATATGGAGTTCCGCGTTACATAAC |
| | TTACGGTAAATGGCCCGCCTGGCTGACCGCCCAACGACCCCCGCC |
| | CATTGACGTCAATAATGACGTATGTTCCCATAGTAACGCCAATAG |
| | GGACTTTCCATTGACGTCAATGGGTGGAGTATTTACGGTAAACTG |
| | CCCACTTGGCAGTACATCAAGTGTATCATATGCCAAGTACGCCCC |
| | CTATTGACGTCAATGACGGTAAATGGCCCGCCTGGCATTATGCCC |
| | AGTACATGACCTTATGGGACTTTCCTACTTGGCAGTACATCTACG |
| | TATTAGTCATCGCTATTACCATGGTGATGCGGTTTTGGCAGTACA |
| | TCAATGGGCGTGGATAGCGGTTTGACTCACGGGGATTTCCAAGTC |
| | TCCACCCCATTGACGTCAATGGGAGTTTGTTTTGGCACCAAAATC |
| | AACGGGACTTTCCAAAATGTCGTAACAACTCCGCCCCATTGACGC |
| | AAATGGGCGGTAGGCGTGTACGGTGGGAGGTCTATATAAGCAGA |
| | GCTC (SEQ ID NO: 18) |
| EF1a | GGCTCCGGTGCCCGTCAGTGGGCAGAGCGCACATCGCCCACAGT |
| | CCCCGAGAAGTTGGGGGGAGGGGTCGGCAATTGAACCGGTGCCT |
| | AGAGAAGGTGGCGCGGGGTAAACTGGGAAAGTGATGCCGTGTAC |
| | TGGCTCCGCCTTTTTCCCGAGGGTGGGGGAGAACCGTATATAAGT |
| | GCAGTAGTCGCCGTGAACGTTCTTTTTCGCAACGGGTTTGCCGCC |
| | AGAACACAGGTAAGTGCCGTGTGTGGTTCCCGCGGGCCTGGCCTC |
| | TTTACGGGTTATGGCCCTTGCGTGCCTTGAATTACTTCCACCTGGC |
| | TGCAGTACGTGATTCTTGATCCCGAGCTTCGGGTTGGAAGTGGGT |
| | GGGAGAGTTCGAGGCCTTGCGCTTAAGGAGCCCCTTCGCCTCGTG |
| | CTTGAGTTGAGGCCTGGCCTGGGCGCTGGGGCCGCCGCGTGCGA |
| | ATCTGGTGGCACCTTCGCGCCTGTCTCGCTGCTTTCGATAAGTCTC |
| | TAGCCATTTAAAATTTTTGATGACCTGCTGCGACGCTTTTTTTCTG |
| | GCAAGATAGTCTTGTAAATGCGGGCCAAGATCTGCACACTGGTAT |
| | TTCGGTTTTTGGGGCCGCGGGCGGCGACGGGGCCCGTGCGTCCCA |
| | GCGCACATGTTCGGCGAGGCGGGGCCTGCGAGCGCGACCACCGA |
| | GAATCGGACGGGGGTAGTCTCAAGCTGGCCGGCCTGCTCTGGTGC |
| | CTGTCCTCGCGCCGCCGTGTATCGCCCCGCCCCGGGCGGCAAGGC |
| | TGGCCCGGTCGGCACCAGTTGCGTGAGCGGAAAGATGGCCGCTT |
| | CCCGGTCCTGCTGCAGGGAGCTCAAAATGGAGGACGCGGCGCTC |
| | GGGAGAGCGGGCGGGTGAGTCACCCACACAAAGGAAAAGGGCC |
| | TTTCCGTCCTCAGCCGTCGCTTCATGTGACTCCACGGAGTACCGG |
| | GCGCCGTCCAGGCACCTCGATTAGTTCTCGAGCTTTTGGAGTACG |
| | TCGTCTTTAGGTTGGGGGGAGGGGTTTTATGCGATGGAGTTTCCC |
| | CACACTGAGTGGGTGGAGACTGAAGTTAGGCCAGCTTGGCACTT |
| | GATGTAATTCTCCTTGGAATTTGCCCTTTTTGAGTTTGGATCTTGG |
| | TTCATTCTCAAGCCTCAGACAGTGGTTCAAAGTTTTTTTCTTCCAT |
| | TTCAGGTGTCGTGA (SEQ ID NO: 19) |
| EFS | GGATCTGCGATCGCTCCGGTGCCCGTCAGTGGGCAGAGCGCACAT |
| | CGCCCACAGTCCCCGAGAAGTTGGGGGGAGGGGTCGGCAATTGA |
| | ACCGGTGCCTAGAGAAGGTGGCGCGGGGTAAACTGGGAAAGTGA |
| | TGTCGTGTACTGGCTCCGCCTTTTTCCCGAGGGTGGGGGAGAACC |
| | GTATATAAGTGCAGTAGTCGCCGTGAACGTTCTTTTTCGCAACGG |
| | GTTTGCCGCCAGAACACAGCTGAAGCTTCGAGGGGCTCGCATCTC |
| | TCCTTCACGCGCCCGCCGCCCTACCTGAGGCCGCCATCCACGCCG |
| | GTTGAGTCGCGTTCTGCCGCCTCCCGCCTGTGGTGCCTCCTGAACT |
| | GCGTCCGCCGTCTAGGTAAGTTTAAAGCTCAGGTCGAGACCGGGC |
| | CTTTGTCCGGCGCTCCCTTGGAGCCTACCTAGACTCAGCCGGCTC |
| | TCCACGCTTTGCCTGACCCTGCTTGCTCAACTCTACGTCTTTGTTT |
| | CGTTTTCTGTTCTGCGCCGTTACAGATCCAAGCTGTGACCGGCGC |
| | CTAC (SEQ ID NO: 20) |
| MND | TTTATTTAGTCTCCAGAAAAAGGGGGGAATGAAAGACCCCACCT |
| | GTAGGTTTGGCAAGCTAGGATCAAGGTTAGGAACAGAGAGACAG |
| | CAGAATATGGGCCAAACAGGATATCTGTGGTAAGCAGTTCCTGCC |
| | CCGGCTCAGGGCCAAGAACAGTTGGAACAGCAGAATATGGGCCA |
| | AACAGGATATCTGTGGTAAGCAGTTCCTGCCCCGGCTCAGGGCCA |
| | AGAACAGATGGTCCCCAGATGCGGTCCCGCCCTCAGCAGTTTCTA |
| | GAGAACCATCAGATGTTTCCAGGGTGCCCCAAGGACCTGAAATG |
| | ACCCTGTGCCTTATTTGAACTAACCAATCAGTTCGCTTCTCGCTTC |
| | TGTTCGCGCGCTTCTGCTCCCCGAGCTCAATAAAAGAGCCCA |
| | (SEQ ID NO: 21) |
| PGK | GGGGTTGGGGTTGCGCCTTTTCCAAGGCAGCCCTGGGTTTGCGCA |
| | GGGACGCGGCTGCTCTGGGCGTGGTTCCGGGAAACGCAGCGGCG |
| | CCGACCCTGGGTCTCGCACATTCTTCACGTCCGTTCGCAGCGTCA |
| | CCCGGATCTTCGCCGCTACCCTTGTGGGCCCCCCGGCGACGCTTC |
| | CTGCTCCGCCCCTAAGTCGGGAAGGTTCCTTGCGGTTCGCGGCGT |
| | GCCGGACGTGACAAACGGAAGCCGCACGTCTCACTAGTACCCTC |
| | GCAGACGGACAGCGCCAGGGAGCAATGGCAGCGCGCCGACCGCG |
| | ATGGGCTGTGGCCAATAGCGGCTGCTCAGCGGGGCGCGCCGAGA |
| | GCAGCGGCCGGGAAGGGGCGGTGCGGGAGGCGGGGTGTGGGGC |
| | GGTAGTGTGGGCCCTGTTCCTGCCCGCGCGGTGTTCCGCATTCTG |
| | CAAGCCTCCGGAGCGCACGTCGGCAGTCGGCTCCCTCGTTGACCG |
| | AATCACCGACCTCTCTCCCCAG (SEQ ID NO: 22) |
| SFFV | GTAACGCCATTTTGCAAGGCATGGAAAAATACCAAACCAAGAAT |
| | AGAGAAGTTCAGATCAAGGGCGGGTACATGAAAATAGCTAACGT |
| | TGGGCCAAACAGGATATCTGCGGTGAGCAGTTTCGGCCCCGGCCC |
| | GGGGCCAAGAACAGATGGTCACCGCAGTTTCGGCCCCGGCCCGA |
| | GGCCAAGAACAGATGGTCCCCAGATATGGCCCAACCCTCAGCAG |
| | TTTCTTAAGACCCATCAGATGTTTCCAGGCTCCCCCAAGGACCTG |
| | AAATGACCCTGCGCCTTATTTGAATTAACCAATCAGCCTGCTTCT |
| | CGCTTCTGTTCGCGCGCTTCTGCTTCCCGAGCTCTATAAAAGAGCT |
| | CACAACCCCTCACTCGGCGCGCCAGTCCTCCGACAGACTGAGTCG |
| | CCCGGG (SEQ ID NO: 23) |
| SV40 | CTGTGGAATGTGTGTCAGTTAGGGTGTGGAAAGTCCCCAGGCTCC |
| | CCAGCAGGCAGAAGTATGCAAAGCATGCATCTCAATTAGTCAGC |
| | AACCAGGTGTGGAAAGTCCCCAGGCTCCCCAGCAGGCAGAAGTA |
| | TGCAAAGCATGCATCTCAATTAGTCAGCAACCATAGTCCCGCCCC |
| | TAACTCCGCCCATCCCGCCCCTAACTCCGCCCAGTTCCGCCCATTC |
| | TCCGCCCCATGGCTGACTAATTTTTTTTATTTATGCAGAGGCCGAG |
| | GCCGCCTCTGCCTCTGAGCTATTCCAGAAGTAGTGAGGAGGCTTT |
| | TTTGGAGGCCTAGGCTTTTGCAAAAAGCT (SEQ ID NO: 24) |
| UbC | GCGCCGGGTTTTGGCGCCTCCCGCGGGCGCCCCCCTCCTCACGGC |
| | GAGCGCTGCCACGTCAGACGAAGGGCGCAGGAGCGTTCCTGATC |
| | CTTCCGCCCGGACGCTCAGGACAGCGGCCCGCTGCTCATAAGACT |
| | CGGCCTTAGAACCCCAGTATCAGCAGAAGGACATTTTAGGACGG |
| | GACTTGGGTGACTCTAGGGCACTGGTTTTCTTTCCAGAGAGCGGA |
| | ACAGGCGAGGAAAAGTAGTCCCTTCTCGGCGATTCTGCGGAGGG |
| | ATCTCCGTGGGGCGGTGAACGCCGATGATTATATAAGGACGCGC |
| | CGGGTGTGGCACAGCTAGTTCCGTCGCAGCCGGGATTTGGGTCGC |
| | GGTTCTTGTTTGTGGATCGCTGTGATCGTCACTTGGTGAGTTGCGG |
| | GCTGCTGGGCTGGCCGGGGCTTTCGTGGCCGCCGGGCCGCTCGGT |
| | GGGACGGAAGCGTGTGGAGAGACCGCCAAGGGCTGTAGTCTGGG |
| | TCCGCGAGCAAGGTTGCCCTGAACTGGGGGTTGGGGGGAGCGCA |
| | CAAAATGGCGGCTGTTCCCGAGTCTTGAATGGAAGACGCTTGTAA |
| | GGCGGGCTGTGAGGTCGTTGAAACAAGGTGGGGGGCATGGTGGG |
| | CGGCAAGAACCCAAGGTCTTGAGGCCTTCGCTAATGCGGGAAAG |
| | CTCTTATTCGGGTGAGATGGGCTGGGGCACCATCTGGGGACCCTG |
| | ACGTGAAGTTTGTCACTGACTGGAGAACTCGGGTTTGTCGTCTGG |
| | TTGCGGGGGCGGCAGTTATGCGGTGCCGTTGGGCAGTGCACCCGT |
| | ACCTTTGGGAGCGCGCGCCTCGTCGTGTCGTGACGTCACCCGTTC |
| | TGTTGGCTTATAATGCAGGGTGGGGCCACCTGCCGGTAGGTGTGC |
| | GGTAGGCTTTTCTCCGTCGCAGGACGCAGGGTTCGGGCCTAGGGT |
| | AGGCTCTCCTGAATCGACAGGCGCCGGACCTCTGGTGAGGGGAG |
| | GGATAAGTGAGGCGTCAGTTTCTTTGGTCGGTTTTATGTACCTATC |
| | TTCTTAAGTAGCTGAAGCTCCGGTTTTGAACTATGCGCTCGGGGT |
| | TGGCGAGTGTGTTTTGTGAAGTTTTTTAGGCACCTTTTGAAATGTA |
| | ATCATTTGGGTCAATATGTAATTTTCAGTGTTAGACTAGTAAAGC |
| | TTCTGCAGGTCGACTCTAGAAAATTGTCCGCTAAATTCTGGCCGT |
| | TTTTGGCTTTTTTGTTAGAC (SEQ ID NO: 25) |
| hEF1aV1 | GGCTCCGGTGCCCGTCAGTGGGCAGAGCGCACATCGCCCACAGT |
| | CCCCGAGAAGTTGGGGGGAGGGGTCGGCAATTGAACCGGTGCCT |
| | AGAGAAGGTGGCGCGGGGTAAACTGGGAAAGTGATGTCGTGTAC |
| | TGGCTCCGCCTTTTTCCCGAGGGTGGGGGAGAACCGTATATAAGT |
| | GCAGTAGTCGCCGTGAACGTTCTTTTTCGCAACGGGTTTGCCGCC |
| | AGAACACAGGTAAGTGCCGTGTGTGGTTCCCGCGGGCCTGGCCTC |
| | TTTACGGGTTATGGCCCTTGCGTGCCTTGAATTACTTCCACCTGGC |
| | TGCAGTACGTGATTCTTGATCCCGAGCTTCGGGTTGGAAGTGGGT |
| | GGGAGAGTTCGAGGCCTTGCGCTTAAGGAGCCCCTTCGCCTCGTG |
| | CTTGAGTTGAGGCCTGGCCTGGGCGCTGGGGCCGCCGCGTGCGA |
| | ATCTGGTGGCACCTTCGCGCCTGTCTCGCTGCTTTCGATAAGTCTC |
| | TAGCCATTTAAAATTTTTGATGACCTGCTGCGACGCTTTTTTTCTG |
| | GCAAGATAGTCTTGTAAATGCGGGCCAAGATCTGCACACTGGTAT |
| | TTCGGTTTTTGGGGCCGCGGGCGGCGACGGGGCCCGTGCGTCCCA |
| | GCGCACATGTTCGGCGAGGCGGGGCCTGCGAGCGCGGCCACCGA |
| | GAATCGGACGGGGGTAGTCTCAAGCTGGCCGGCCTGCTCTGGTGC |
| | CTGGTCTCGCGCCGCCGTGTATCGCCCCGCCCTGGGCGGCAAGGC |
| | TGGCCCGGTCGGCACCAGTTGCGTGAGCGGAAAGATGGCCGCTT |
| | CCCGGCCCTGCTGCAGGGAGCTCAAAATGGAGGACGCGGCGCTC |
| | GGGAGAGCGGGCGGGTGAGTCACCCACACAAAGGAAAAGGGCC |
| | TTTCCGTCCTCAGCCGTCGCTTCATGTGACTCCACGGAGTACCGG |
| | GCGCCGTCCAGGCACCTCGATTAGTTCTCGAGCTTTTGGAGTACG |
| | TCGTCTTTAGGTTGGGGGGAGGGGTTTTATGCGATGGAGTTTCCC |
| | CACACTGAGTGGGTGGAGACTGAAGTTAGGCCAGCTTGGCACTT |
| | GATGTAATTCTCCTTGGAATTTGCCCTTTTTGAGTTTGGATCTTGG |
| | TTCATTCTCAAGCCTCAGACAGTGGTTCAAAGTTTTTTTCTTCCAT |
| | TTCAGGTGTCGTGA (SEQ ID NO: 26) |
| hCAGG | ACTAGTTATTAATAGTAATCAATTACGGGGTCATTAGTTCATAGC |
| | CCATATATGGAGTTCCGCGTTACATAACTTACGGTAAATGGCCCG |
| | CCTGGCTGACCGCCCAACGACCCCCGCCCATTGACGTCAATAATG |
| | ACGTATGTTCCCATAGTAACGCCAATAGGGACTTTCCATTGACGT |
| | CAATGGGTGGAGTATTTACGGTAAACTGCCCACTTGGCAGTACAT |
| | CAAGTGTATCATATGCCAAGTACGCCCCCTATTGACGTCAATGAC |
| | GGTAAATGGCCCGCCTGGCATTATGCCCAGTACATGACCTTATGG |
| | GACTTTCCTACTTGGCAGTACATCTACGTATTAGTCATCGCTATTA |
| | CCATGGTCGAGGTGAGCCCCACGTTCTGCTTCACTCTCCCCATCTC |
| | CCCCCCCTCCCCACCCCCAATTTTGTATTTATTTATTTTTTAATTAT |
| | TTTGTGCAGCGATGGGGGCGGGGGGGGGGGGGGGGCGCGCGCCA |
| | GGCGGGGCGGGGCGGGGCGAGGGGGGGGGCGGGGCGAGGCGGA |
| | GAGGTGCGGCGGCAGCCAATCAGAGCGGCGCGCTCCGAAAGTTT |
| | CCTTTTATGGCGAGGCGGCGGCGGCGGCGGCCCTATAAAAAGCG |
| | AAGCGCGCGGCGGGGGGGAGTCGCTGCGACGCTGCCTTCGCCC |
| | CGTGCCCCGCTCCGCCGCCGCCTCGCGCCGCCCGCCCCGGCTCTG |
| | ACTGACCGCGTTACTCCCACAGGTGAGCGGGCGGGACGGCCCTTC |
| | TCCTCCGGGCTGTAATTAGCGCTTGGTTTAATGACGGCTTGTTTCT |
| | TTTCTGTGGCTGCGTGAAAGCCTTGAGGGGCTCCGGGAGGGCCCT |
| | TTGTGCGGGGGGAGCGGCTCGGGGGGTGCGTGCGTGTGTGTGTG |
| | CGTGGGGAGCGCCGCGTGCGGCTCCGCGCTGCCCGGCGGCTGTG |
| | AGCGCTGCGGGCGCGGCGCGGGGCTTTGTGCGCTCCGCAGTGTGC |
| | GCGAGGGGAGCGCGGCCGGGGGCGGTGCCCCGCGGTGCGGGGG |
| | GGGCTGCGAGGGGAACAAAGGCTGCGTGCGGGGTGTGTGCGTGG |
| | GGGGGTGAGCAGGGGGTGTGGGCGCGTCGGTCGGGCTGCAACCC |
| | CCCCTGCACCCCCCTCCCCGAGTTGCTGAGCACGGCCCGGCTTCG |
| | GGTGCGGGGCTCCGTACGGGGCGTGGCGCGGGGCTCGCCGTGCC |
| | GGGCGGGGGGTGGCGGCAGGTGGGGGTGCCGGGCGGGGCGGGG |
| | CCGCCTCGGGCCGGGGAGGGCTCGGGGGAGGGGCGCGGCGGCCC |
| | CCGGAGCGCCGGCGGCTGTCGAGGCGCGGCGAGCCGCAGCCATT |
| | GCCTTTTATGGTAATCGTGCGAGAGGGCGCAGGGACTTCCTTTGT |
| | CCCAAATCTGTGCGGAGCCGAAATCTGGGAGGCGCCGCCGCACC |
| | CCCTCTAGCGGGCGCGGGGCGAAGCGGTGCGGCGCCGGCAGGAA |
| | GGAAATGGGCGGGGAGGGCCTTCGTGCGTCGCCGCGCCGCCGTC |
| | CCCTTCTCCCTCTCCAGCCTCGGGGCTGTCCGCGGGGGGACGGCT |
| | GCCTTCGGGGGGGACGGGGCAGGGCGGGGTTCGGCTTCTGGCGT |
| | GTGACCGGCGGCTCTAGAGCCTCTGCTAACCATGTTCATGCCTTC |
| | TTCTTTTTCCTACAGCTCCTGGGCAACGTGCTGGTTATTGTGCTGT |
| | CTCATCATTTTGGCAAAGAATTC (SEQ ID NO: 27) |
| hEF1aV2 | GGGCAGAGCGCACATCGCCCACAGTCCCCGAGAAGTTGGGGGGA |
| | GGGGTCGGCAATTGAACCGGTGCCTAGAGAAGGTGGCGCGGGGT |
| | AAACTGGGAAAGTGATGTCGTGTACTGGCTCCGCCTTTTTCCCGA |
| | GGGTGGGGGAGAACCGTATATAAGTGCAGTAGTCGCCGTGAACG |
| | TTCTTTTTCGCAACGGGTTTGCCGCCAGAACACAG (SEQ ID NO: |
| | 28) |
| hACTb | CCACTAGTTCCATGTCCTTATATGGACTCATCTTTGCCTATTGCGA |
| | CACACACTCAATGAACACCTACTACGCGCTGCAAAGAGCCCCGC |
| | AGGCCTGAGGTGCCCCCACCTCACCACTCTTCCTATTTTTGTGTAA |
| | AAATCCAGCTTCTTGTCACCACCTCCAAGGAGGGGGAGGAGGAG |
| | GAAGGCAGGTTCCTCTAGGCTGAGCCGAATGCCCCTCTGTGGTCC |
| | CACGCCACTGATCGCTGCATGCCCACCACCTGGGTACACACAGTC |
| | TGTGATTCCCGGAGCAGAACGGACCCTGCCCACCCGGTCTTGTGT |
| | GCTACTCAGTGGACAGACCCAAGGCAAGAAAGGGTGACAAGGAC |
| | AGGGTCTTCCCAGGCTGGCTTTGAGTTCCTAGCACCGCCCCGCCC |
| | CCAATCCTCTGTGGCACATGGAGTCTTGGTCCCCAGAGTCCCCCA |
| | GCGGCCTCCAGATGGTCTGGGAGGGCAGTTCAGCTGTGGCTGCGC |
| | ATAGCAGACATACAACGGACGGTGGGCCCAGACCCAGGCTGTGT |
| | AGACCCAGCCCCCCCGCCCCGCAGTGCCTAGGTCACCCACTAACG |
| | CCCCAGGCCTGGTCTTGGCTGGGCGTGACTGTTACCCTCAAAAGC |
| | AGGCAGCTCCAGGGTAAAAGGTGCCCTGCCCTGTAGAGCCCACC |
| | TTCCTTCCCAGGGCTGCGGCTGGGTAGGTTTGTAGCCTTCATCAC |
| | GGGCCACCTCCAGCCACTGGACCGCTGGCCCCTGCCCTGTCCTGG |
| | GGAGTGTGGTCCTGCGACTTCTAAGTGGCCGCAAGCCACCTGACT |
| | CCCCCAACACCACACTCTACCTCTCAAGCCCAGGTCTCTCCCTAG |
| | TGACCCACCCAGCACATTTAGCTAGCTGAGCCCCACAGCCAGAG |
| | GTCCTCAGGCCCTGCTTTCAGGGCAGTTGCTCTGAAGTCGGCAAG |
| | GGGGAGTGACTGCCTGGCCACTCCATGCCCTCCAAGAGCTCCTTC |
| | TGCAGGAGCGTACAGAACCCAGGGCCCTGGCACCCGTGCAGACC |
| | CTGGCCCACCCCACCTGGGCGCTCAGTGCCCAAGAGATGTCCACA |
| | CCTAGGATGTCCCGCGGTGGGTGGGGGGCCCGAGAGACGGGCAG |
| | GCCGGGGGCAGGCCTGGCCATGCGGGGCCGAACCGGGCACTGCC |
| | CAGCGTGGGGCGCGGGGGCCACGGCGCGCGCCCCCAGCCCCCGG |
| | GCCCAGCACCCCAAGGCGGCCAACGCCAAAACTCTCCCTCCTCCT |
| | CTTCCTCAATCTCGCTCTCGCTCTTTTTTTTTTTCGCAAAAGGAGG |
| | GGAGAGGGGGTAAAAAAATGCTGCACTGTGCGGCGAAGCCGGTG |
| | AGTGAGCGGCGCGGGGCCAATCAGCGTGCGCCGTTCCGAAAGTT |
| | GCCTTTTATGGCTCGAGCGGCCGCGGCGGCGCCCTATAAAACCCA |
| | GCGGCGCGACGCGCCACCACCGCCGAGACCGCGTCCGCCCCGCG |
| | AGCACAGAGCCTCGCCTTTGCCGATCCGCCGCCCGTCCACACCCG |
| | CCGCCAGGTAAGCCCGGCCAGCCGACCGGGGCAGGCGGCTCACG |
| | GCCCGGCCGCAGGCGGCCGCGGCCCCTTCGCCCGTGCAGAGCCG |
| | CCGTCTGGGCCGCAGCGGGGGGCGCATGGGGGGGGAACCGGACC |
| | GCCGTGGGGGGCGCGGGAGAAGCCCCTGGGCCTCCGGAGATGGG |
| | GGACACCCCACGCCAGTTCGGAGGCGCGAGGCCGCGCTCGGGAG |
| | GCGCGCTCCGGGGGTGCCGCTCTCGGGGCGGGGGCAACCGGCGG |
| | GGTCTTTGTCTGAGCCGGGCTCTTGCCAATGGGGATCGCAGGGTG |
| | GGCGCGGCGGAGCCCCCGCCAGGCCCGGTGGGGGCTGGGGCGCC |
| | ATTGCGCGTGCGCGCTGGTCCTTTGGGCGCTAACTGCGTGCGCGC |
| | TGGGAATTGGCGCTAATTGCGCGTGCGCGCTGGGACTCAAGGCG |
| | CTAACTGCGCGTGCGTTCTGGGGCCCGGGGTGCCGCGGCCTGGGC |
| | TGGGGCGAAGGCGGGCTCGGCCGGAAGGGGTGGGGTCGCCGCGG |
| | CTCCCGGGCGCTTGCGCGCACTTCCTGCCCGAGCCGCTGGCCGCC |
| | CGAGGGTGTGGCCGCTGCGTGCGCGCGCGCCGACCCGGCGCTGTT |
| | TGAACCGGGCGGAGGCGGGGCTGGCGCCCGGTTGGGAGGGGGTT |
| | GGGGCCTGGCTTCCTGCCGCGCGCCGCGGGGACGCCTCCGACCA |
| | GTGTTTGCCTTTTATGGTAATAACGCGGCCGGCCCGGCTTCCTTTG |
| | TCCCCAATCTGGGCGCGCGCCGGCGCCCCCTGGCGGCCTAAGGAC |
| | TCGGCGCGCCGGAAGTGGCCAGGGCGGGGGCGACCTCGGCTCAC |
| | AGCGCGCCCGGCTAT (SEQ ID NO: 29) |
| heIF4A1 | GTTGATTTCCTTCATCCCTGGCACACGTCCAGGCAGTGTCGAATC |
| | CATCTCTGCTACAGGGGAAAACAAATAACATTTGAGTCCAGTGG |
| | AGACCGGGAGCAGAAGTAAAGGGAAGTGATAACCCCCAGAGCCC |
| | GGAAGCCTCTGGAGGCTGAGACCTCGCCCCCCTTGCGTGATAGGG |
| | CCTACGGAGCCACATGACCAAGGCACTGTCGCCTCCGCACGTGTG |
| | AGAGTGCAGGGCCCCAAGATGGCTGCCAGGCCTCGAGGCCTGAC |
| | TCTTCTATGTCACTTCCGTACCGGCGAGAAAGGCGGGCCCTCCAG |
| | CCAATGAGGCTGCGGGGGGGGCCTTCACCTTGATAGGCACTCGA |
| | GTTATCCAATGGTGCCTGCGGGCCGGAGCGACTAGGAACTAACG |
| | TCATGCCGAGTTGCTGAGCGCCGGCAGGCGGGGCCGGGGCGGCC |
| | AAACCAATGCGATGGCCGGGGCGGAGTCGGGCGCTCTATAAGTT |
| | GTCGATAGGCGGGCACTCCGCCCTAGTTTCTAAGGACCATG (SEQ |
| | ID NO: 30) |
| hGAPDH | AGTTCCCCAACTTTCCCGCCTCTCAGCCTTTGAAAGAAAGAAAGG |
| | GGAGGGGGCAGGCCGCGTGCAGTCGCGAGCGGTGCTGGGCTCCG |
| | GCTCCAATTCCCCATCTCAGTCGCTCCCAAAGTCCTTCTGTTTCAT |
| | CCAAGCGTGTAAGGGTCCCCGTCCTTGACTCCCTAGTGTCCTGCT |
| | GCCCACAGTCCAGTCCTGGGAACCAGCACCGATCACCTCCCATCG |
| | GGCCAATCTCAGTCCCTTCCCCCCTACGTCGGGGCCCACACGCTC |
| | GGTGCGTGCCCAGTTGAACCAGGCGGCTGCGGAAAAAAAAAAGC |
| | GGGGAGAAAGTAGGGCCCGGCTACTAGCGGTTTTACGGGCGCAC |
| | GTAGCTCAGGCCTCAAGACCTTGGGCTGGGACTGGCTGAGCCTGG |
| | CGGGAGGCGGGGTCCGAGTCACCGCCTGCCGCCGCGCCCCCGGT |
| | TTCTATAAATTGAGCCCGCAGCCTCCCGCTTCGCTCTCTGCTCCTC |
| | CTGTTCGACAGTCAGCCGCATCTTCTTTTGCGTCGCCAGGTGAAG |
| | ACGGGCGGAGAGAAACCCGGGAGGCTAGGGACGGCCTGAAGGC |
| | GGCAGGGGCGGGCGCAGGCCGGATGTGTTCGCGCCGCTGCGGGG |
| | TGGGCCCGGGCGGCCTCCGCATTGCAGGGGGGGGCGGAGGACGT |
| | GATGCGGCGCGGGCTGGGCATGGAGGCCTGGTGGGGGAGGGGAG |
| | GGGAGGCGTGGGTGTCGGCCGGGGCCACTAGGCGCTCACTGTTCT |
| | CTCCCTCCGCGCAGCCGAGCCACATCGCTGAGACAC (SEQ ID NO: |
| | 31) |
| hGRP78 | AGTGCGGTTACCAGCGGAAATGCCTCGGGGTCAGAAGTCGCAGG |
| | AGAGATAGACAGCTGCTGAACCAATGGGACCAGCGGATGGGGCG |
| | GATGTTATCTACCATTGGTGAACGTTAGAAACGAATAGCAGCCAA |
| | TGAATCAGCTGGGGGGGCGGAGCAGTGACGTTTATTGCGGAGGG |
| | GGCCGCTTCGAATCGGCGGCGGCCAGCTTGGTGGCCTGGGCCAAT |
| | GAACGGCCTCCAACGAGCAGGGCCTTCACCAATCGGCGGCCTCC |
| | ACGACGGGGCTGGGGGAGGGTATATAAGCCGAGTAGGCGACGGT |
| | GAGGTCGACGCCGGCCAAGACAGCACAGACAGATTGACCTATTG |
| | GGGTGTTTCGCGAGTGTGAGAGGGAAGCGCCGCGGCCTGTATTTC |
| | TAGACCTGCCCTTCGCCTGGTTCGTGGCGCCTTGTGACCCCGGGC |
| | CCCTGCCGCCTGCAAGTCGGAAATTGCGCTGTGCTCCTGTGCTAC |
| | GGCCTGTGGCTGGACTGCCTGCTGCTGCCCAACTGGCTGGCAC |
| | (SEQ ID NO: 157) |
| hGRP94 | TAGTTTCATCACCACCGCCACCCCCCCGCCCCCCCGCCATCTGAA |
| | AGGGTTCTAGGGGATTTGCAACCTCTCTCGTGTGTTTCTTCTTTCC |
| | GAGAAGCGCCGCCACACGAGAAAGCTGGCCGCGAAAGTCGTGCT |
| | GGAATCACTTCCAACGAAACCCCAGGCATAGATGGGAAAGGGTG |
| | AAGAACACGTTGCCATGGCTACCGTTTCCCCGGTCACGGAATAAA |
| | CGCTCTCTAGGATCCGGAAGTAGTTCCGCCGCGACCTCTCTAAAA |
| | GGATGGATGTGTTCTCTGCTTACATTCATTGGACGTTTTCCCTTAG |
| | AGGCCAAGGCCGCCCAGGCAAAGGGGCGGTCCCACGCGTGAGGG |
| | GCCCGCGGAGCCATTTGATTGGAGAAAAGCTGCAAACCCTGACC |
| | AATCGGAAGGAGCCACGCTTCGGGCATCGGTCACCGCACCTGGA |
| | CAGCTCCGATTGGTGGACTTCCGCCCCCCCTCACGAATCCTCATT |
| | GGGTGCCGTGGGTGCGTGGTGCGGCGCGATTGGTGGGTTCATGTT |
| | TCCCGTCCCCCGCCCGCGAGAAGTGGGGGTGAAAAGCGGCCCGA |
| | CCTGCTTGGGGTGTAGTGGGCGGACCGCGCGGCTGGAGGTGTGA |
| | GGATCCGAACCCAGGGGTGGGGGGTGGAGGCGGCTCCTGCGATC |
| | GAAGGGGACTTGAGACTCACCGGCCGCACGTC (SEQ ID NO: 33) |
| hHSP70 | GGGCCGCCCACTCCCCCTTCCTCTCAGGGTCCCTGTCCCCTCCAGT |
| | GAATCCCAGAAGACTCTGGAGAGTTCTGAGCAGGGGGCGGCACT |
| | CTGGCCTCTGATTGGTCCAAGGAAGGCTGGGGGGCAGGACGGGA |
| | GGCGAAAACCCTGGAATATTCCCGACCTGGCAGCCTCATCGAGCT |
| | CGGTGATTGGCTCAGAAGGGAAAAGGCGGGTCTCCGTGACGACT |
| | TATAAAAGCCCAGGGGCAAGCGGTCCGGATAACGGCTAGCCTGA |
| | GGAGCTGCTGCGACAGTCCACTACCTTTTTCGAGAGTGACTCCCG |
| | TTGTCCCAAGGCTTCCCAGAGCGAACCTGTGCGGCTGCAGGCACC |
| | GGCGCGTCGAGTTTCCGGCGTCCGGAAGGACCGAGCTCTTCTCGC |
| | GGATCCAGTGTTCCGTTTCCAGCCCCCAATCTCAGAGCGGAGCCG |
| | ACAGAGAGCAGGGAACCC (SEQ ID NO: 34) |
| hKINb | GCCCCACCCCCGTCCGCGTTACAACCGGGAGGCCCGCTGGGTCCT |
| | GCACCGTCACCCTCCTCCCTGTGACCGCCCACCTGATACCCAAAC |
| | AACTTTCTCGCCCCTCCAGTCCCCAGCTCGCCGAGCGCTTGCGGG |
| | GAGCCACCCAGCCTCAGTTTCCCCAGCCCCGGGCGGGGCGAGGG |
| | GCGATGACGTCATGCCGGCGCGCGGCATTGTGGGGCGGGGCGAG |
| | GCGGGGCGCCGGGGGGAGCAACACTGAGACGCCATTTTCGGCGG |
| | CGGGAGCGGCGCAGGCGGCCGAGCGGGACTGGCTGGGTCGGCTG |
| | GGCTGCTGGTGCGAGGAGCCGCGGGGCTGTGCTCGGCGGCCAAG |
| | GGGACAGCGCGTGGGTGGCCGAGGATGCTGCGGGGCGGTAGCTC |
| | CGGCGCCCCTCGCTGGTGACTGCTGCGCCGTGCCTCACACAGCCG |
| | AGGCGGGCTCGGCGCACAGTCGCTGCTCCGCGCTCGCGCCCGGC |
| | GGCGCTCCAGGTGCTGACAGCGCGAGAGAGCGCGGCCTCAGGAG |
| | CAACAC (SEQ ID NO: 35) |
| hUBIb | TTCCAGAGCTTTCGAGGAAGGTTTCTTCAACTCAAATTCATCCGC |
| | CTGATAATTTTCTTATATTTTCCTAAAGAAGGAAGAGAAGCGCAT |
| | AGAGGAGAAGGGAAATAATTTTTTAGGAGCCTTTCTTACGGCTAT |
| | GAGGAATTTGGGGCTCAGTTGAAAAGCCTAAACTGCCTCTCGGG |
| | AGGTTGGGCGCGGCGAACTACTTTCAGCGGCGCACGGAGACGGC |
| | GTCTACGTGAGGGGTGATAAGTGACGCAACACTCGTTGCATAAAT |
| | TTGCGCTCCGCCAGCCCGGAGCATTTAGGGGCGGTTGGCTTTGTT |
| | GGGTGAGCTTGTTTGTGTCCCTGTGGGTGGACGTGGTTGGTGATT |
| | GGCAGGATCCTGGTATCCGCTAACAGGTACTGGCCCACAGCCGTA |
| | AAGACCTGCGGGGGCGTGAGAGGGGGGAATGGGTGAGGTCAAG |
| | CTGGAGGCTTCTTGGGGTTGGGTGGGCCGCTGAGGGGAGGGGAG |
| | GGCGAGGTGACGCGACACCCGGCCTTTCTGGGAGAGTGGGCCTT |
| | GTTGACCTAAGGGGGGCGAGGGCAGTTGGCACGCGCACGCGCCG |
| | ACAGAAACTAACAGACATTAACCAACAGCGATTCCGTCGCGTTTA |
| | CTTGGGAGGAAGGCGGAAAAGAGGTAGTTTGTGTGGCTTCTGGA |
| | AACCCTAAATTTGGAATCCCAGTATGAGAATGGTGTCCCTTCTTG |
| | TGTTTCAATGGGATTTTTACTTCGCGAGTCTTGTGGGTTTGGTTTT |
| | GTTTTCAGTTTGCCTAACACCGTGCTTAGGTTTGAGGCAGATTGG |
| | AGTTCGGTCGGGGGAGTTTGAATATCCGGAACAGTTAGTGGGGA |
| | AAGCTGTGGACGCTTGGTAAGAGAGCGCTCTGGATTTTCCGCTGT |
| | TGACGTTGAAACCTTGAATGACGAATTTCGTATTAAGTGACTTAG |
| | CCTTGTAAAATTGAGGGGAGGCTTGCGGAATATTAACGTATTTAA |
| | GGCATTTTGAAGGAATAGTTGCTAATTTTGAAGAATATTAGGTGT |
| | AAAAGCAAGAAATACAATGATCCTGAGGTGACACGCTTATGTTTT |
| | ACTTTTAAACTAGGTCACC (SEQ ID NO: 36) |

In some embodiments, engineered polynucleotides or constructs of the present disclosure are configured to produce multiple polypeptides. For example, polynucleotides may be configured to produce 2 different polypeptides. The polynucleotide may be configured to produce a polypeptide including polypeptides of the inducible cell death systems including a modified ER-LBD described herein, e.g., a first polypeptide monomer and/or a second polypeptide monomer that each include the modified ER-LBD and a cell death inducing domain.

In some embodiments, polypeptides of the inducible cell death systems including a modified ER-LBD described herein, e.g., a first polypeptide monomer and/or a second polypeptide monomer that each include the modified ER-LBD and a cell death inducing domain, may be encoded by the same polynucleotide or heterologous construct.

In some embodiments, engineered nucleic acids can be multicistronic, i.e., more than one separate polypeptide (e.g., multiple exogenous polypeptides, such as a first polypeptide monomer and a second polypeptide monomer that each include the modified ER-LBD and a cell death inducing domain) can be produced from a single transcript. Engineered nucleic acids can be multicistronic through the use of various linkers, e.g., a polynucleotide sequence encoding a first exogenous polynucleotide can be linked to a nucleotide sequence encoding a second exogenous polynucleotide, such as in a first gene: linker: second gene 5′ to 3′ orientation. A linker polynucleotide sequence can encode one or more 2A ribosome skipping elements, such as T2A. Other 2A ribosome skipping elements include, but are not limited to, E2A, P2A, and F2A. 2A ribosome skipping elements allow production of separate polypeptides encoded by the first and second genes are produced during translation. A linker can encode a cleavable linker polypeptide sequence, such as a Furin cleavage site or a TEV cleavage site, wherein following expression the cleavable linker polypeptide is cleaved such that separate polypeptides encoded by the first and second genes are produced. A cleavable linker can include a polypeptide sequence, such as such a flexible linker (e.g., a Gly-Ser-Gly sequence), that further promotes cleavage.

A linker can encode an Internal Ribosome Entry Site (IRES), such that separate polypeptides encoded by the first and second genes are produced during translation. A linker can encode a splice acceptor, such as a viral splice acceptor.

A linker can be a combination of linkers, such as a Furin-2A linker that can produce separate polypeptides through 2A ribosome skipping followed by further cleavage of the Furin site to allow for complete removal of 2A residues. In some embodiments, a combination of linkers can include a Furin sequence, a flexible linker, and 2A linker. Accordingly, in some embodiments, the linker is a Furin-Gly-Ser-Gly-2A fusion polypeptide. In some embodiments, a linker is a Furin-Gly-Ser-Gly-T2A fusion polypeptide.

In general, a multicistronic system can use any number or combination of linkers, to express any number of genes or portions thereof (e.g., an engineered nucleic acid can encode a first, a second, and a third polypeptide molecule, each separated by linkers such that separate polypeptides encoded by the first, second, and third polypeptides are produced).

“Linkers,” as used herein, can refer to polypeptides that link a first polypeptide sequence and a second polypeptide sequence or the multicistronic linkers described above.

### Expression Systems Further Including a Target Expression Cassette

In some aspects, the chimeric protein is a chimeric transcription factor and the present disclosure further provides a target expression cassette including a chimeric transcription factor-responsive (CTF-responsive) promoter. For example, chimeric transcription factors having modified ER-LDBs may be used to regulate expression of pro-cell-death/apoptotic factors (e.g., a polypeptide including a cell death-inducing domain), cell survival polypeptides, inhibitors of pro-apoptotic factors, and/or inhibitors of cell survival polypeptides.

“Target expression cassette” refers to an expression cassette including a gene with chimeric transcription factor-controllable expression. The expression is controlled by the chimeric transcription factor based on the presence of a non-endogenous ligand (e.g., 4-OHT or endoxifen).

In some aspects, the present disclosure provides polynucleotides encoding a gene of interest operably linked to a chimeric transcription factor-responsive promoter (CTF-responsive promoter). CTF-responsive promoters are synthetic, inducible promoters that are responsive to a chimeric transcription factor including a modified ER-LBD, and are inducible in response to a non-endogenous ligand such as 4-OHT.

In some embodiments, the CTF-responsive promoter comprises a core promoter sequence and a binding domain that binds to a chimeric transcription factor as described herein.

The binding domain may include one or more zinc finger binding sites. The binding domain can comprise 1, 2, 3, 4, 5, 6, 7, 8, 9, 10, or more zinc finger binding sites. In some embodiments, the binding domain comprises one zinc finger binding site. In some embodiments, the binding domain comprises two zinc finger binding sites. In some embodiments, the binding domain comprises three zinc finger binding sites. In some embodiments, the binding domain comprises four zinc finger binding sites. An exemplary binding domain comprising zinc finger binding sites is shown in the sequence:

(SEQ ID NO: 37)
CGGGTTTCGTAACAATCGCATGAGGATTCGCAACGCCTTCGGCGTAGCCG
ATGTCGCGCTCCCGTCTCAGTAAAGGTCGGCGTAGCCGATGTCGCGCAAT
CGGACTGCCTTCGTACGGCGTAGCCGATGTCGCGCGTATCAGTCGCCTCG
GAACGGCGTAGCCGATGTCGCGCATTCGTAAGAGGCTCACTCTCCCTTAC
ACGGAGTGGATAACTAGTTCTAGAGGGTATATAATGGGGGCCA.

The core promoter sequence may include a minimal promoter. Examples of minimal promoters include minP, minCMV, YB_TATA, and minTK.

In some aspects, the chimeric protein including the modified ER-LBD is a chimeric transcription factor, and the heterologous construct further includes a target expression cassette including a chimeric-transcription factor responsive promoter. In some aspects, provided herein is a first heterologous construct comprising an expression cassette that comprises a polynucleotide that encodes chimeric transcription factor including a modified ER-LBD, and a second heterologous construct comprising a target expression cassette including a chimeric transcription factor-responsive (CTF-responsive) promoter.

### Post-Transcriptional Regulatory Elements

In some embodiments, an engineered nucleic acid of the present disclosure comprises a post-transcriptional regulatory element (PRE). PREs can enhance gene expression via enabling tertiary RNA structure stability and 3′ end formation. Non-limiting examples of PREs include the Hepatitis B virus PRE (HPRE) and the Woodchuck Hepatitis Virus PRE (WPRE). In some embodiments, the post-transcriptional regulatory element is a Woodchuck Hepatitis Virus Posttranscriptional Regulatory Element (WPRE). In some embodiments, the WPRE comprises the alpha, beta, and gamma components of the WPRE element. In some embodiments, the WPRE comprises the alpha component of the WPRE element. Examples of WPRE sequences include SEQ ID NO: 38 and SEQ ID NO: 39.

### Engineered Cells

Also provided herein are cells, and methods of producing cells, that comprise one or more polynucleotides or constructs of the present disclosure. These cells are referred to herein as “engineered cells.” These cells, which typically contain one or more engineered nucleic acids, do not occur in nature. In some embodiments, the cells are isolated cells that recombinantly express the one or more engineered polynucleotides. In some embodiments, the engineered polynucleotides are expressed from one or more vectors or a selected locus from the genome of the cell. In some embodiments, the cells are engineered to include a polynucleotide comprising a promoter operably linked to a nucleotide sequence.

An engineered cell of the present disclosure can comprise an engineered polynucleotide integrated into the cell's genome. An engineered cell can comprise an engineered polynucleotide capable of expression without integrating into the cell's genome, for example, engineered with a transient expression system such as a plasmid or mRNA.

### Engineered Cell Types

An engineered cell of the present disclosure can be a human cell. An engineered cell can be a human primary cell. An engineered primary cell can be any somatic cell. An engineered primary cell can be any stem cell. In some embodiments, the engineered cell is derived from the subject. In some embodiments, the engineered cell is allogeneic with reference to the subject.

An engineered cell of the present disclosure can be isolated from a subject, such as a subject known or suspected to have cancer. Cell isolation methods are known to those skilled in the art and include, but are not limited to, sorting techniques based on cell-surface marker expression, such as FACS sorting, positive isolation techniques, and negative isolation, magnetic isolation, and combinations thereof. An engineered cell can be allogenic with reference to the subject being administered a treatment. Allogenic modified cells can be HLA-matched to the subject being administered a treatment. An engineered cell can be a cultured cell, such as an ex vivo cultured cell. An engineered cell can be an ex vivo cultured cell, such as a primary cell isolated from a subject. Cultured cell can be cultured with one or more cytokines.

In some embodiments, an engineered cell of the present disclosure is selected from: a T cell (e.g., a CD8+ T cell, a CD4+ T cell, or a gamma-delta T cell), a cytotoxic T lymphocyte (CTL), a regulatory T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage (e.g., an M1 macrophage or an M2 macrophage), a monocyte, a dendritic cell, an erythrocyte, a platelet cell, a neuron, an oligodendrocyte, an astrocyte, a placode-derived cell, a Schwann cell, a cardiomyocyte, an endothelial cell, a nodal cell, a microglial cell, a hepatocyte, a cholangiocyte, a beta cell, a human embryonic stem cell (ESC), an ESC-derived cell, a pluripotent stem cell, a mesenchymal stromal cell (MSC), an induced pluripotent stem cell (iPSC), and an iPSC-derived cell.

In some embodiments, an engineered cell of the present disclosure is a T cell (e.g., a CD8+ T cell, a CD4+ T cell, or a gamma-delta T cell). In some embodiments, an engineered of the present disclosure is a cytotoxic T lymphocyte (CTL). In some embodiments, an engineered cell of the present disclosure is a regulatory T cell. In some embodiments, an engineered cell of the present disclosure is a Natural Killer T (NKT) cell. In some embodiments, an engineered cell of the present disclosure is a Natural Killer (NK) cell. In some embodiments, an engineered cell of the present disclosure is a B cell. In some embodiments, an engineered cell of the present disclosure is a tumor-infiltrating lymphocyte (TIL). In some embodiments, an engineered cell of the present disclosure is an innate lymphoid cell. In some embodiments, an engineered cell of the present disclosure is a mast cell. In some embodiments, an engineered cell of the present disclosure is an eosinophil. In some embodiments, an engineered cell of the present disclosure is a basophil. In some embodiments, an engineered cell of the present disclosure is a neutrophil. In some embodiments, an engineered cell of the present disclosure is a myeloid cell. In some embodiments, an engineered cell of the present disclosure is a macrophage e.g., an M1 macrophage or an M2 macrophage). In some embodiments, an engineered cell of the present disclosure is a monocyte. In some embodiments, an engineered or isolated cell of the present disclosure is a dendritic cell. In some embodiments, an engineered cell of the present disclosure is an erythrocyte. In some embodiments, an engineered cell of the present disclosure is a platelet cell. In some embodiments, a cell of the present disclosure is a neuron. In some embodiments, a cell of the present disclosure is an oligodendrocyte. In some embodiments, a cell of the present disclosure is an astrocyte. In some embodiments, a cell of the present disclosure is a placode-derived cell. In some embodiments, an engineered cell of the present disclosure is a Schwann cell. In some embodiments, an engineered cell of the present disclosure is a cardiomyocyte. In some embodiments, an engineered cell of the present disclosure is an endothelial cell. In some embodiments, an engineered cell of the present disclosure is a nodal cell. In some embodiments, an engineered cell of the present disclosure is a microglial cell. In some embodiments, an engineered cell of the present disclosure is a hepatocyte. In some embodiments, an engineered cell of the present disclosure is a cholangiocyte. In some embodiments, an engineered cell of the present disclosure is a beta cell. In some embodiments, an engineered cell of the present disclosure is a human embryonic stem cell (ESC). In some embodiments, an engineered cell of the present disclosure is an ESC-derived cell. In some embodiments, an engineered cell of the present disclosure is a pluripotent stem cell. In some embodiments, an engineered cell of the present disclosure is a mesenchymal stromal cell (MSC). In some embodiments, an engineered cell of the present disclosure is an induced pluripotent stem cell (iPSC). In some embodiments, an engineered cell of the present disclosure is an iPSC-derived cell. In some embodiments, an engineered cell is autologous. In some embodiments, an engineered cell is allogeneic. In some embodiments, an engineered cell of the present disclosure is a CD34+ cell, a CD3+ cell, a CD8+ cell, a CD16+ cell, and/or a CD4+ cell.

In some embodiments, a cell of the present disclosure is a tumor cell selected from: an adenocarcinoma cell, a bladder tumor cell, a brain tumor cell, a breast tumor cell, a cervical tumor cell, a colorectal tumor cell, an esophageal tumor cell, a glioma cell, a kidney tumor cell, a liver tumor cell, a lung tumor cell, a melanoma cell, a mesothelioma cell, an ovarian tumor cell, a pancreatic tumor cell, a prostate tumor cell, a skin tumor cell, a thyroid tumor cell, and a uterine tumor cell.

Also provided herein are methods that include culturing the engineered cells of the present disclosure. Methods of culturing the engineered cells described herein are known. One skilled in the art will recognize that culturing conditions will depend on the particular engineered cell of interest. One skilled in the art will recognize that culturing conditions will depend on the specific downstream use of the engineered cell, for example, specific culturing conditions for subsequent administration of the engineered cell to a subject.

### Methods of Engineering Cells

Also provided herein are compositions and methods for engineering cells with any polynucleotide or construct as described herein.

In general, cells are engineered through introduction (i.e., delivery) of one or more polynucleotides of the present disclosure. Delivery methods include, but are not limited to, viral-mediated delivery, lipid-mediated transfection, nanoparticle delivery, electroporation, sonication, and cell membrane deformation by physical means. One skilled in the art will appreciate the choice of delivery method can depend on the specific cell type to be engineered.

### Viral-Mediated Delivery

Viral vector-based delivery platforms can be used to engineer cells. In general, a viral vector-based delivery platform engineers a cell through introducing (i.e., delivering) into a host cell. For example, a viral vector-based delivery platform can engineer a cell through introducing any of the engineered nucleic acids described herein. A viral vector-based delivery platform can be a nucleic acid, and as such, an engineered nucleic acid can also encompass an engineered virally derived nucleic acid. Such engineered virally derived nucleic acids can also be referred to as recombinant viruses or engineered viruses.

A viral vector-based delivery platform can encode more than one engineered nucleic acid, gene, or transgene within the same nucleic acid. For example, an engineered virally derived nucleic acid, e.g., a recombinant virus or an engineered virus, can encode one or more transgenes, including, but not limited to, any of the engineered nucleic acids described herein. The one or more transgenes can be configured to express polypeptides described herein (e.g., inducible cell death systems including a modified ER-LBD). A viral vector-based delivery platform can encode one or more genes in addition to the transgene encoding the modified ER-LBD, such as viral genes needed for viral infectivity and/or viral production (e.g., capsid proteins, envelope proteins, viral polymerases, viral transcriptases, etc.), referred to as cis-acting elements or genes.

A viral vector-based delivery platform can comprise more than one viral vector, such as separate viral vectors encoding the engineered nucleic acids, genes, or transgenes described herein, and referred to as trans-acting elements or genes. For example, a helper-dependent viral vector-based delivery platform can provide additional genes needed for viral infectivity and/or viral production on one or more additional separate vectors in addition to the vector encoding the modified ER-LBD. One viral vector can deliver more than one engineered polynucleotides, such as one vector that delivers an engineered polynucleotide configured to produce a modified ER-LBD and an engineered polynucleotide configured produce a gene of interest. More than one viral vector can deliver more than one engineered nucleic acids, such as a first vector that delivers an engineered polynucleotide configured to produce a modified ER-LBD and a second vector that delivers an additional engineered polynucleotide. The number of viral vectors used can depend on the packaging capacity of the above-mentioned viral vector-based vaccine platforms, and one skilled in the art can select the appropriate number of viral vectors.

In general, any of the viral vector-based systems can be used for the in vitro production of molecules, or used in vivo and ex vivo gene therapy procedures, e.g., for in vivo delivery. The selection of an appropriate viral vector-based system will depend on a variety of factors, such as cargo/payload size, immunogenicity of the viral system, target cell of interest, gene expression strength and timing, and other factors appreciated by one skilled in the art.

Viral vector-based delivery platforms can be RNA-based viruses or DNA-based viruses. Exemplary viral vector-based delivery platforms include, but are not limited to, a herpes simplex virus, an adenovirus, a measles virus, an influenza virus, a Indiana vesiculovirus, a Newcastle disease virus, a vaccinia virus, a poliovirus, a myxoma virus, a reovirus, a mumps virus, a Maraba virus, a rabies virus, a rotavirus, a hepatitis virus, a rubella virus, a dengue virus, a chikungunya virus, a respiratory syncytial virus, a lymphocytic choriomeningitis virus, a morbillivirus, a lentivirus, a replicating retrovirus, a rhabdovirus, a Seneca Valley virus, a sindbis virus, and any variant or derivative thereof. Other exemplary viral vector-based delivery platforms are described in the art, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239 (1): 45-61, Sakuma et al., Lentiviral vectors: basic to translational, Biochem J. (2012) 443 (3): 603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In vivo Gene Delivery, J. Virol. (1998) 72 (12): 9873-9880).

The sequences may be preceded with one or more sequences targeting a subcellular compartment. Upon introduction (i.e., delivery) into a host cell, infected cells (i.e., an engineered cell) can express, and in some case secrete, the modified ER-LBD (or chimeric polypeptide including the modified ER-LBD). Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vectors useful for the introduction (i.e., delivery) of engineered nucleic acids, e.g., Salmonella typhi vectors, and the like will be apparent to those skilled in the art from the description herein.

The viral vector-based delivery platforms can be a virus that targets a tumor cell, herein referred to as an oncolytic virus. Examples of oncolytic viruses include, but are not limited to, an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof. Any of the oncolytic viruses described herein can be a recombinant oncolytic virus comprising one more transgenes (e.g., an engineered nucleic acid described herein). The transgenes can be configured to express a modified ER-LBD (or chimeric polypeptide including the modified ER-LBD) and optionally a gene of interest.

In some embodiments, the virus is selected from: a lentivirus, a retrovirus, an oncolytic virus, an adenovirus, an adeno-associated virus (AAV), and a virus-like particle (VLP).

The viral vector-based delivery platform can be retrovirus-based. In general, retroviral vectors are comprised of cis-acting long terminal repeats with packaging capacity for up to 6-10 kb of foreign sequence. The minimum cis-acting LTRs are sufficient for replication and packaging of the vectors, which are then used to integrate the one or more engineered nucleic acids (e.g., a transgene encoding the modified ER-LBD) into the target cell to provide permanent transgene expression. Retroviral-based delivery systems include, but are not limited to, those based upon murine leukemia, virus (MuLV), gibbon ape leukemia virus (GaLV), Simian Immunodeficiency virus (SIV), human immunodeficiency virus (HIV), and combinations thereof (see, e.g., Buchscher et al., J. Virol. 66:2731-2739 (1992); Johann et ah, J. Virol. 66:1635-1640 (1992); Sommnerfelt et al., Virol. 176:58-59 (1990); Wilson et ah, J. Virol. 63:2374-2378 (1989); Miller et al, J, Virol. 65:2220-2224 (1991); PCT/US94/05700). Other retroviral systems include the Phoenix retrovirus system.

The viral vector-based delivery platform can be lentivirus-based. In general, lentiviral vectors are retroviral vectors that are able to transduce or infect non-dividing cells and typically produce high viral titers. Lentiviral-based delivery platforms can be HIV-based, such as ViraPower systems (ThermoFisher) or pLenti systems (Cell Biolabs). Lentiviral-based delivery platforms can be SIV, or FIV-based. Other exemplary lentivirus-based delivery platforms are described in more detail in U.S. Pat. Nos. 7,311,907; 7,262,049; 7,250,299; 7,226,780; 7,220,578; 7,211,247; 7,160,721; 7,078,031; 7,070,993; 7,056,699; 6,955,919, each herein incorporated by reference for all purposes.

The viral vector-based delivery platform can be adenovirus-based. In general, adenoviral based vectors are capable of very high transduction efficiency in many cell types, do not require cell division, achieve high titer and levels of expression, and can be produced in large quantities in a relatively simple system. In general, adenoviruses can be used for transient expression of a transgene within an infected cell since adenoviruses do not typically integrate into a host's genome. Adenovirus-based delivery platforms are described in more detail in Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655, each herein incorporated by reference for all purposes. Other exemplary adenovirus-based delivery platforms are described in more detail in U.S. Pat. Nos. 5,585,362; 6,083,716, 7,371,570; 7,348,178; 7,323,177; 7,319,033; 7,318,919; and 7,306,793 and International Patent Application WO96/13597, each herein incorporated by reference for all purposes.

The viral vector-based delivery platform can be adeno-associated virus (AAV)-based. Adeno-associated virus (“AAV”) vectors may be used to transduce cells with engineered nucleic acids (e.g., any of the engineered nucleic acids described herein). AAV systems can be used for the in vitro production of a modified ER-LBD (or chimeric polypeptide including the modified ER-LBD), or used in vivo and ex vivo gene therapy procedures, e.g., for in vivo delivery of the modified ER-LBD (see, e.g., West et al., Virology 160:38-47 (1987); U.S. Pat. Nos. 4,797,368; 5,436,146; 6,632,670; 6,642,051; 7,078,387; 7,314,912; 6,498,244; 7,906,111; US patent publications US 2003-0138772, US 2007/0036760, and US 2009/0197338; Gao, et al., J. Virol, 78 (12): 6381-6388 (June 2004); Gao, et al, Proc Natl Acad Sci USA, 100 (10): 6081-6086 (May 13, 2003); and International Patent applications WO 2010/138263 and WO 93/24641; Kotin, Human Gene Therapy 5:793-801 (1994); Muzyczka, J. Clin. Invest. 94:1351 (1994), each herein incorporated by reference for all purposes). Exemplary methods for constructing recombinant AAV vectors are described in more detail in U.S. Pat. No. 5,173,414; Tratschin et ah, Mol. Cell. Biol. 5:3251-3260 (1985); Tratschin, et ah, Mol. Cell, Biol. 4:2072-2081 (1984); Hermonat &amp; Muzyczka, PNAS 81:64666470 (1984); and Samuiski et ah, J. Virol. 63:03822-3828 (1989), each herein incorporated by reference for all purposes. In general, an AAV-based vector comprises a capsid protein having an amino acid sequence corresponding to any one of AAV1, AAV2, AAV3, AAV4, AAV5, AAV6, AAV7, AAV8, AAV9, AAV.Rh10, AAV11 and variants thereof.

The viral vector-based delivery platform can be a virus-like particle (VLP) platform. In general, VLPs are constructed by producing viral structural proteins and purifying resulting viral particles. Then, following purification, a cargo/payload (e.g., any of the engineered nucleic acids described herein) is encapsulated within the purified particle ex vivo. Accordingly, production of VLPs maintains separation of the nucleic acids encoding viral structural proteins and the nucleic acids encoding the cargo/payload. The viral structural proteins used in VLP production can be produced in a variety of expression systems, including mammalian, yeast, insect, bacterial, or in vivo translation expression systems. The purified viral particles can be denatured and reformed in the presence of the desired cargo to produce VLPs using methods known to those skilled in the art. Production of VLPs are described in more detail in Seow et al. (Mol Ther. 2009 May; 17 (5): 767-777), herein incorporated by reference for all purposes.

The viral vector-based delivery platform can be engineered to target (i.e., infect) a range of cells, target a narrow subset of cells, or target a specific cell. In general, the envelope protein chosen for the viral vector-based delivery platform will determine the viral tropism. The virus used in the viral vector-based delivery platform can be pseudotyped to target a specific cell of interest. The viral vector-based delivery platform can be pantropic and infect a range of cells. For example, pantropic viral vector-based delivery platforms can include the VSV-G envelope. The viral vector-based delivery platform can be amphotropic and infect mammalian cells. Accordingly, one skilled in the art can select the appropriate tropism, pseudotype, and/or envelope protein for targeting a desired cell type.

### Lipid Structure Delivery Systems

Engineered nucleic acids of the present disclosure (e.g., a nucleic acid encoding a modified ER-LBD or chimeric protein described herein) can be introduced into a cell using a lipid-mediated delivery system. In general, a lipid-mediated delivery system uses a structure composed of an outer lipid membrane enveloping an internal compartment. Examples of lipid-based structures include, but are not limited to, a lipid-based nanoparticle, a liposome, a micelle, an exosome, a vesicle, an extracellular vesicle, a cell, or a tissue. Lipid structure delivery systems can deliver a cargo/payload (e.g., any of the engineered nucleic acids described herein) in vitro, in vivo, or ex vivo.

A lipid-based nanoparticle can include, but is not limited to, a unilamellar liposome, a multilamellar liposome, and a lipid preparation. As used herein, a “liposome” is a generic term encompassing in vitro preparations of lipid vehicles formed by enclosing a desired cargo, e.g., an engineered nucleic acid, such as any of the engineered nucleic acids described herein, within a lipid shell or a lipid aggregate. Liposomes may be characterized as having vesicular structures with a bilayer membrane, generally comprising a phospholipid, and an inner medium that generally comprises an aqueous composition. Liposomes include, but are not limited to, emulsions, foams, micelles, insoluble monolayers, liquid crystals, phospholipid dispersions, lamellar layers and the like. Liposomes can be unilamellar liposomes. Liposomes can be multilamellar liposomes. Liposomes can be multivesicular liposomes. Liposomes can be positively charged, negatively charged, or neutrally charged. In certain embodiments, the liposomes are neutral in charge. Liposomes can be formed from standard vesicle-forming lipids, which generally include neutral and negatively charged phospholipids and a sterol, such as cholesterol. The selection of lipids is generally guided by consideration of a desired purpose, e.g., criteria for in vivo delivery, such as liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369, each herein incorporated by reference for all purposes.

A multilamellar liposome is generated spontaneously when lipids comprising phospholipids are suspended in an excess of aqueous solution such that multiple lipid layers are separated by an aqueous medium. Water and dissolved solutes are entrapped in closed structures between the lipid bilayers following the lipid components undergoing self-rearrangement. A desired cargo (e.g., a polypeptide, a nucleic acid, a small molecule drug, an engineered nucleic acid, such as any of the engineered nucleic acids described herein, a viral vector, a viral-based delivery system, etc.) can be encapsulated in the aqueous interior of a liposome, attached to a liposome via a linking molecule that is associated with both the liposome and the polypeptide/nucleic acid, interspersed within the lipid bilayer of a liposome, entrapped in a liposome, complexed with a liposome, or otherwise associated with the liposome such that it can be delivered to a target entity. Lipophilic molecules or molecules with lipophilic regions may also dissolve in or associate with the lipid bilayer.

A liposome used according to the present embodiments can be made by different methods, as would be known to one of ordinary skill in the art. Preparations of liposomes are described in further detail in WO 2016/201323, International Applications PCT/US85/01161 and PCT/US89/05040, and U.S. Pat. Nos. 4,728,578, 4,728,575, 4,737,323, 4,533,254, 4,162,282, 4,310,505, and 4,921,706; each herein incorporated by reference for all purposes.

Liposomes can be cationic liposomes. Examples of cationic liposomes are described in more detail in U.S. Pat. Nos. 5,962,016; 5,030,453; 6,680,068, U.S. Application 2004/0208921, and International Patent Applications WO03/015757A1, WO04029213A2, and WO02/100435A1, each hereby incorporated by reference in their entirety.

Lipid-mediated gene delivery methods are described, for instance, in WO 96/18372; WO 93/24640; Mannino & Gould-Fogerite, BioTechniques 6 (7): 682-691 (1988); U.S. Pat. No. 5,279,833 Rose U.S. Pat. No. 5,279,833; WO91/06309; and Felgner et al., Proc. Natl. Acad. Sci. USA 84:7413-7414 (1987), each herein incorporated by reference for all purposes.

Exosomes are small membrane vesicles of endocytic origin that are released into the extracellular environment following fusion of multivesicular bodies with the plasma membrane. The size of exosomes ranges between 30 and 100 nm in diameter. Their surface consists of a lipid bilayer from the donor cell's cell membrane, and they contain cytosol from the cell that produced the exosome, and exhibit membrane proteins from the parental cell on the surface. Exosomes useful for the delivery of nucleic acids are known to those skilled in the art, e.g., the exosomes described in more detail in U.S. Pat. No. 9,889,210, herein incorporated by reference for all purposes.

As used herein, the term “extracellular vesicle” or “EV” refers to a cell-derived vesicle comprising a membrane that encloses an internal space. In general, extracellular vesicles comprise all membrane-bound vesicles that have a smaller diameter than the cell from which they are derived. Generally extracellular vesicles range in diameter from 20 nm to 1000 nm, and can comprise various macromolecular cargo either within the internal space, displayed on the external surface of the extracellular vesicle, and/or spanning the membrane. The cargo can comprise nucleic acids (e.g., any of the engineered nucleic acids described herein), proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells.

As used herein the term “exosome” refers to a cell-derived small (between 20-300 nm in diameter, more preferably 40-200 nm in diameter) vesicle comprising a membrane that encloses an internal space, and which is generated from the cell by direct plasma membrane budding or by fusion of the late endosome with the plasma membrane. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The exosome can be derived from a producer cell, and isolated from the producer cell based on its size, density, biochemical parameters, or a combination thereof. An exosome is a species of extracellular vesicle. Generally, exosome production/biogenesis does not result in the destruction of the producer cell. Exosomes and preparation of exosomes are described in further detail in WO 2016/201323, which is hereby incorporated by reference in its entirety.

As used herein, the term “nanovesicle” (also referred to as a “microvesicle”) refers to a cell-derived small (between 20-250 nm in diameter, more preferably 30-150 nm in diameter) vesicle comprising a membrane that encloses an internal space, and which is generated from the cell by direct or indirect manipulation such that said nanovesicle would not be produced by said producer cell without said manipulation. In general, a nanovesicle is a sub-species of an extracellular vesicle. Appropriate manipulations of the producer cell include but are not limited to serial extrusion, treatment with alkaline solutions, sonication, or combinations thereof. The production of nanovesicles may, in some instances, result in the destruction of said producer cell. Preferably, populations of nanovesicles are substantially free of vesicles that are derived from producer cells by way of direct budding from the plasma membrane or fusion of the late endosome with the plasma membrane. The nanovesicle comprises lipid or fatty acid and polypeptide, and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The nanovesicle, once it is derived from a producer cell according to said manipulation, may be isolated from the producer cell based on its size, density, biochemical parameters, or a combination thereof.

Lipid nanoparticles (LNPs), in general, are synthetic lipid structures that rely on the amphiphilic nature of lipids to form membranes and vesicle like structures (Riley 2017). In general, these vesicles deliver cargo/payloads, such as any of the engineered nucleic acids or viral systems described herein, by absorbing into the membrane of target cells and releasing the cargo into the cytosol. Lipids used in LNP formation can be cationic, anionic, or neutral. The lipids can be synthetic or naturally derived, and in some instances biodegradable. Lipids can include fats, cholesterol, phospholipids, lipid conjugates including, but not limited to, polyethyleneglycol (PEG) conjugates (PEGylated lipids), waxes, oils, glycerides, and fat soluble vitamins. Lipid compositions generally include defined mixtures of materials, such as the cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl-4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids. In addition, LNPs can be further engineered or functionalized to facilitate targeting of specific cell types. Another consideration in LNP design is the balance between targeting efficiency and cytotoxicity.

Micelles, in general, are spherical synthetic lipid structures that are formed using single-chain lipids, where the single-chain lipid's hydrophilic head forms an outer layer or membrane and the single-chain lipid's hydrophobic tails form the micelle center. Micelles typically refer to lipid structures only containing a lipid mono-layer. Micelles are described in more detail in Quader et al. (Mol Ther. 2017 Jul. 5; 25 (7): 1501-1513), herein incorporated by reference for all purposes.

Nucleic-acid vectors, such as expression vectors, exposed directly to serum can have several undesirable consequences, including degradation of the nucleic acid by serum nucleases or off-target stimulation of the immune system by the free nucleic acids. Similarly, viral delivery systems exposed directly to serum can trigger an undesired immune response and/or neutralization of the viral delivery system. Therefore, encapsulation of an engineered nucleic acid and/or viral delivery system can be used to avoid degradation, while also avoiding potential off-target effects. In certain examples, an engineered nucleic acid and/or viral delivery system is fully encapsulated within the delivery vehicle, such as within the aqueous interior of an LNP. Encapsulation of an engineered nucleic acid and/or viral delivery system within an LNP can be carried out by techniques well-known to those skilled in the art, such as microfluidic mixing and droplet generation carried out on a microfluidic droplet generating device. Such devices include, but are not limited to, standard T-junction devices or flow-focusing devices. In an example, the desired lipid formulation, such as MC3 or MC3-like containing compositions, is provided to the droplet generating device in parallel with an engineered nucleic acid or viral delivery system and any other desired agents, such that the delivery vector and desired agents are fully encapsulated within the interior of the MC3 or MC3-like based LNP. In an example, the droplet generating device can control the size range and size distribution of the LNPs produced. For example, the LNP can have a size ranging from 1 to 1000 nanometers in diameter, e.g., 1, 10, 50, 100, 500, or 1000 nanometers. Following droplet generation, the delivery vehicles encapsulating the cargo/payload (e.g., an engineered nucleic acid and/or viral delivery system) can be further treated or engineered to prepare them for administration.

### Nanoparticle Delivery

Nanomaterials can be used to deliver engineered nucleic acids (e.g., a nucleic acid encoding a modified ER-LBD or chimeric protein described herein). Nanomaterial vehicles, importantly, can be made of non-immunogenic materials and generally avoid eliciting immunity to the delivery vector itself. These materials can include, but are not limited to, lipids (as previously described), inorganic nanomaterials, and other polymeric materials. Nanomaterial particles are described in more detail in Riley et al. (Recent Advances in Nanomaterials for Gene Delivery-A Review. Nanomaterials 2017, 7 (5), 94), herein incorporated by reference for all purposes.

### Genomic Editing Systems

Genomic editing systems can be used to engineer a host genome to encode an engineered nucleic acid, such as a nucleic acid encoding a modified ER-LBD of the present disclosure. In general, a “genomic editing system” refers to any system for integrating an exogenous gene into a host cell's genome. Genomic editing systems include, but are not limited to, a transposon system, a nuclease genomic editing system, and a viral vector-based delivery platform.

A transposon system can be used to integrate an engineered nucleic acid, such as an engineered nucleic acid of the present disclosure, into a host genome. Transposons generally comprise terminal inverted repeats (TIR) that flank a cargo/payload nucleic acid and a transposase. The transposon system can provide the transposon in cis or in trans with the TIR-flanked cargo. A transposon system can be a retrotransposon system or a DNA transposon system. In general, transposon systems integrate a cargo/payload (e.g., an engineered nucleic acid) randomly into a host genome. Examples of transposon systems include systems using a transposon of the Tc1/mariner transposon superfamily, such as a Sleeping Beauty transposon system, described in more detail in Hudecek et al. (Crit Rev Biochem Mol Biol. 2017 August; 52 (4): 355-380), and U.S. Pat. Nos. 6,489,458, 6,613,752 and 7,985,739, each of which is herein incorporated by reference for all purposes. Another example of a transposon system includes a PiggyBac transposon system, described in more detail in U.S. Pat. Nos. 6,218,185 and 6,962,810, each of which is herein incorporated by reference for all purposes.

A nuclease genomic editing system can be used to engineer a host genome to encode an engineered nucleic acid, such as an isolated polynucleotide or heterologous construct of the present disclosure. Without wishing to be bound by theory, in general, the nuclease-mediated gene editing systems used to introduce an exogenous gene take advantage of a cell's natural DNA repair mechanisms, particularly homologous recombination (HR) repair pathways. Briefly, following an insult to genomic DNA (typically a double-stranded break), a cell can resolve the insult by using another DNA source that has identical, or substantially identical, sequences at both its 5′ and 3′ ends as a template during DNA synthesis to repair the lesion. In a natural context, HDR can use the other chromosome present in a cell as a template. In gene editing systems, exogenous polynucleotides are introduced into the cell to be used as a homologous recombination template (HRT or HR template). In general, any additional exogenous sequence not originally found in the chromosome with the lesion that is included between the 5′ and 3′ complimentary ends within the HRT (e.g., a gene or a portion of a gene) can be incorporated (i.e., “integrated”) into the given genomic locus during templated HDR. Thus, a typical HR template for a given genomic locus has a nucleotide sequence identical to a first region of an endogenous genomic target locus, a nucleotide sequence identical to a second region of the endogenous genomic target locus, and a nucleotide sequence encoding a cargo/payload nucleic acid (e.g., any of the engineered nucleic acids described herein, such as any of the engineered nucleic acids described herein).

In some examples, a HR template can be linear. Examples of linear HR templates include, but are not limited to, a linearized plasmid vector, a ssDNA, a synthesized DNA, and a PCR amplified DNA. In particular examples, a HR template can be circular, such as a plasmid. A circular template can include a supercoiled template.

The identical, or substantially identical, sequences found at the 5′ and 3′ ends of the HR template, with respect to the exogenous sequence to be introduced, are generally referred to as arms (HR arms). HR arms can be identical to regions of the endogenous genomic target locus (i.e., 100% identical). HR arms in some examples can be substantially identical to regions of the endogenous genomic target locus. While substantially identical HR arms can be used, it can be advantageous for HR arms to be identical as the efficiency of the HDR pathway may be impacted by HR arms having less than 100% identity.

Each HR arm, i.e., the 5′ and 3′ HR arms, can be the same size or different sizes. Each HR arm can each be greater than or equal to 50, 100, 200, 300, 400, or 500 bases in length. Although HR arms can, in general, be of any length, practical considerations, such as the impact of HR arm length and overall template size on overall editing efficiency, can also be taken into account. An HR arms can be identical, or substantially identical to, regions of an endogenous genomic target locus immediately adjacent to a cleavage site. Each HR arms can be identical to, or substantially identical to, regions of an endogenous genomic target locus immediately adjacent to a cleavage site. Each HR arms can be identical, or substantially identical to, regions of an endogenous genomic target locus within a certain distance of a cleavage site, such as 1 base-pair, less than or equal to 10 base-pairs, less than or equal to 50 base-pairs, or less than or equal to 100 base-pairs of each other.

A nuclease genomic editing system can use a variety of nucleases to cut a target genomic locus, including, but not limited to, a Clustered Regularly Interspaced Short Palindromic Repeats (CRISPR) family nuclease or derivative thereof, a Transcription activator-like effector nuclease (TALEN) or derivative thereof, a zinc-finger nuclease (ZFN) or derivative thereof, and a homing endonuclease (HE) or derivative thereof.

A CRISPR-mediated gene editing system can be used to engineer a host genome to encode an engineered nucleic acid, such as an engineered nucleic acid described herein. CRISPR systems are described in more detail in M. Adli (“The CRISPR tool kit for genome editing and beyond” Nature Communications; volume 9 (2018), Article number: 1911), herein incorporated by reference for all that it teaches. In general, a CRISPR-mediated gene editing system comprises a CRISPR-associated (Cas) nuclease and an RNA(s) that directs cleavage to a particular target sequence. An exemplary CRISPR-mediated gene editing system is the CRISPR/Cas9 systems comprised of a Cas9 nuclease and an RNA(s) that has a CRISPR RNA (crRNA) domain and a trans-activating CRISPR (tracrRNA) domain. The crRNA typically has two RNA domains: a guide RNA sequence (gRNA) that directs specificity through base-pair hybridization to a target sequence (“a defined nucleotide sequence”), e.g., a genomic sequence; and an RNA domain that hybridizes to a tracrRNA. A tracrRNA can interact with and thereby promote recruitment of a nuclease (e.g., Cas9) to a genomic locus. The crRNA and tracrRNA polynucleotides can be separate polynucleotides. The crRNA and tracrRNA polynucleotides can be a single polynucleotide, also referred to as a single guide RNA (sgRNA). While the Cas9 system is illustrated here, other CRISPR systems can be used, such as the Cpf1 system. Nucleases can include derivatives thereof, such as Cas9 functional mutants, e.g., a Cas9 “nickase” mutant that in general mediates cleavage of only a single strand of a defined nucleotide sequence as opposed to a complete double-stranded break typically produced by Cas9 enzymes.

In general, the components of a CRISPR system interact with each other to form a Ribonucleoprotein (RNP) complex to mediate sequence specific cleavage. In some CRISPR systems, each component can be separately produced and used to form the RNP complex. In some CRISPR systems, each component can be separately produced in vitro and contacted (i.e., “complexed”) with each other in vitro to form the RNP complex. The in vitro produced RNP can then be introduced (i.e., “delivered”) into a cell's cytosol and/or nucleus, e.g., a T cell's cytosol and/or nucleus. The in vitro produced RNP complexes can be delivered to a cell by a variety of means including, but not limited to, electroporation, lipid-mediated transfection, cell membrane deformation by physical means, lipid nanoparticles (LNP), virus like particles (VLP), and sonication. In a particular example, in vitro produced RNP complexes can be delivered to a cell using a Nucleofactor/Nucleofection® electroporation-based delivery system (Lonza®). Other electroporation systems include, but are not limited to, MaxCyte electroporation systems, Miltenyi CliniMACS electroporation systems, Neon electroporation systems, and BTX electroporation systems. CRISPR nucleases, e.g., Cas9, can be produced in vitro (i.e., synthesized and purified) using a variety of protein production techniques known to those skilled in the art. CRISPR system RNAs, e.g., an sgRNA, can be produced in vitro (i.e., synthesized and purified) using a variety of RNA production techniques known to those skilled in the art, such as in vitro transcription or chemical synthesis.

An in vitro produced RNP complex can be complexed at different ratios of nuclease to gRNA. An in vitro produced RNP complex can also be used at different amounts in a CRISPR-mediated editing system. For example, depending on the number of cells desired to be edited, the total RNP amount added can be adjusted, such as a reduction in the amount of RNP complex added when editing a large number of cells in a reaction.

In some CRISPR systems, each component (e.g., Cas9 and an sgRNA) can be separately encoded by a polynucleotide with each polynucleotide introduced into a cell together or separately. In some CRISPR systems, each component can be encoded by a single polynucleotide (i.e., a multi-promoter or multicistronic vector, see description of exemplary multicistronic systems below) and introduced into a cell. Following expression of each polynucleotide encoded CRISPR component within a cell (e.g., translation of a nuclease and transcription of CRISPR RNAs), an RNP complex can form within the cell and can then direct site-specific cleavage.

Some RNPs can be engineered to have moieties that promote delivery of the RNP into the nucleus. For example, a Cas9 nuclease can have a nuclear localization signal (NLS) domain such that if a Cas9 RNP complex is delivered into a cell's cytosol or following translation of Cas9 and subsequent RNP formation, the NLS can promote further trafficking of a Cas9 RNP into the nucleus.

The cells described herein can be engineered using non-viral methods, e.g., the nuclease and/or CRISPR mediated gene editing systems described herein can be delivered to a cell using non-viral methods. The cells described herein can be engineered using viral methods, e.g., the nuclease and/or CRISPR mediated gene editing systems described herein can be delivered to a cell using viral methods such as adenoviral, retroviral, lentiviral, or any of the other viral-based delivery methods described herein.

In some CRISPR systems, more than one CRISPR composition can be provided such that each separately target the same gene or general genomic locus at more than target nucleotide sequence. For example, two separate CRISPR compositions can be provided to direct cleavage at two different target nucleotide sequences within a certain distance of each other. In some CRISPR systems, more than one CRISPR composition can be provided such that each separately target opposite strands of the same gene or general genomic locus. For example, two separate CRISPR “nickase” compositions can be provided to direct cleavage at the same gene or general genomic locus at opposite strands.

In general, the features of a CRISPR-mediated editing system described herein can apply to other nuclease-based genomic editing systems. TALEN is an engineered site-specific nuclease, which is composed of the DNA-binding domain of TALE (transcription activator-like effectors) and the catalytic domain of restriction endonuclease Fokl. By changing the amino acids present in the highly variable residue region of the monomers of the DNA binding domain, different artificial TALENs can be created to target various nucleotides sequences. The DNA binding domain subsequently directs the nuclease to the target sequences and creates a double-stranded break. TALEN-based systems are described in more detail in U.S. Ser. No. 12/965,590; U.S. Pat. Nos. 8,450,471; 8,440,431; 8,440,432; 10,172,880; and U.S. Ser. No. 13/738,381, all of which are incorporated by reference herein in their entirety. ZFN-based editing systems are described in more detail in U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties for all purposes.

### Other Engineering Delivery Systems

Various additional means to introduce engineered nucleic acids (e.g., an isolated polynucleotide encoding a modified ER-LBD or chimeric protein described herein) into a cell or other target recipient entity, such as any of the lipid structures described herein.

Electroporation can be used to deliver polynucleotides to recipient entities. Electroporation is a method of internalizing a cargo/payload into a target cell or entity's interior compartment through applying an electrical field to transiently permeabilize the outer membrane or shell of the target cell or entity. In general, the method involves placing cells or target entities between two electrodes in a solution containing a cargo of interest (e.g., any of the engineered nucleic acids described herein). The lipid membrane of the cells is then disrupted, i.e., permeabilized, by applying a transient set voltage that allows the cargo to enter the interior of the entity, such as the cytoplasm of the cell. In the example of cells, at least some, if not a majority, of the cells remain viable. Cells and other entities can be electroporated in vitro, in vivo, or ex vivo. Electroporation conditions (e.g., number of cells, concentration of cargo, recovery conditions, voltage, time, capacitance, pulse type, pulse length, volume, cuvette length, electroporation solution composition, etc.) vary depending on several factors including, but not limited to, the type of cell or other recipient entity, the cargo to be delivered, the efficiency of internalization desired, and the viability desired. Optimization of such criteria are within the scope of those skilled in the art. A variety of devices and protocols can be used for electroporation. Examples include, but are not limited to, Neon® Transfection System, MaxCyte® Flow Electroporation™, Lonza® Nucleofector™ systems, and Bio-Rad® electroporation systems.

Other means for introducing engineered nucleic acids (e.g., an isolated polynucleotide encoding a modified ER-LBD or chimeric protein described herein) into a cell or other target recipient entity include, but are not limited to, sonication, gene gun, hydrodynamic injection, and cell membrane deformation by physical means.

Compositions and methods for delivering engineered mRNAs in vivo, such as naked plasmids or mRNA, are described in detail in Kowalski et al. (Mol Ther. 2019 April 10; 27 (4): 710-728) and Kaczmarek et al. (Genome Med. 2017; 9:60.), each herein incorporated by reference for all purposes.

### Methods of Use

Methods of using a modified ER-LBD, chimeric protein, or cell of inducible cell death systems as described herein are also encompassed by this disclosure.

In some aspects, the methods include a method of inducing cell death, including: transforming a cell with (i) a heterologous construct encoding an inducible cell death systems (e.g., any one of the inducible cell death systems described herein) and (ii) contacting the transformed cell with a non-endogenous ligand of the modified estrogen receptor ligand binding domain (ER-LBD).

In some embodiments, the methods include a method of inducing cell death, including: transforming a cell with (i) a heterologous construct encoding an inducible cell-death system including a polypeptide, where the polypeptide includes a ligand binding domain and a cell death inducing domain, where the polypeptide is configured upon contact with a ligand of the ligand binding domain to generate a cell-death inducing signal in a cell in which the polypeptide is expressed, and where the ligand binding domain includes a modified ER-LBD (such as any of the modified ER-LBDs described herein); and (ii) contacting the transformed cell with a non-endogenous ligand of the modified estrogen receptor ligand binding domain (ER-LBD).

In some embodiments, the methods include a method of inducing cell death, including: transforming a cell with (i) an inducible cell-death system including a first polypeptide monomer and a second polypeptide monomer, where the first polypeptide monomer and the second polypeptide monomer each include a ligand binding domain and a cell death inducing domain, wherein the first polypeptide monomer and the second polypeptide monomer are configured to oligomerize with each other upon contact with a ligand of the ligand binding domain, thereby generating the cell-death inducing signal in a cell in which the first polypeptide monomer and the second polypeptide monomer are expressed, and where the ligand binding domain includes a modified ER-LBD (such as any of the modified ER-LBDs described herein); and (ii) contacting the transformed cell with a non-endogenous ligand of the modified estrogen receptor ligand binding domain (ER-LBD).

In some aspects, the methods include modulating transcription of a gene of interest. Methods of modulating transcription may include: transforming a cell with (i) a heterologous construct encoding a chimeric transcription factor that includes a modified ER-LBD, and (ii) a target expression cassette comprising a chimeric transcription factor-responsive (CTF-responsive) promoter operably linked to a gene of interest; culturing the transformed cell under conditions suitable for expression of the chimeric protein; and inducing the chimeric protein to modulate transcription of the gene of interest by contacting the transformed cell with a non-endogenous ligand.

In some embodiments, the method of modulating transcription is a method of activating transcription. Activating transcription may be achieved using a chimeric protein.

In some embodiments, the methods include activating transcription. Activating transcription may be achieved, for example, using a chimeric protein that includes a modified ER-LBD, a DNA binding domain, and a transcriptional activation domain.

In some embodiments, the methods include repressing transcription. Repressing transcription may be achieved, for example, using a chimeric protein that includes a modified ER-LBD, an DNA binding domain, and a transcriptional repressor domain.

In some aspects, the methods include modulating localization of a chimeric protein. Methods of modulating localization may include transforming a cell with a heterologous construct encoding a chimeric protein including a modified ER-LBD domain and a polypeptide of interest; culturing the transformed call under conditions suitable for expression of the chimeric protein; and inducing nuclear localization of the chimeric protein by contacting the transformed cell with a non-endogenous ligand. In some embodiments, modulating localization comprises inducing nuclear localization.

In some embodiments, the non-endogenous ligand is administered at a concentration at which the non-endogenous ligand is substantially inactive on wild-type estrogen receptor alpha.

### In Vivo Methods

The methods provided herein also include in vivo methods, e.g., for inducing cell death, inducing oligomerization of a chimeric protein provided herein, modifying localization and/or modulating transcription in vivo, e.g., by delivering a non-endogenous ligand to a cell expressing the modified ER-LBD or chimeric protein in vivo.

In some embodiments, the transformed cell is in a human or animal, and contacting the transformed cell with the non-endogenous ligand comprises administering a pharmacological dose of the ligand to the human or animal. In some embodiments, the non-endogenous ligand administered to the subject comprises tamoxifen. Upon oral administration of tamoxifen, the drug is converted in the liver to an active tamoxifen metabolite. In some embodiments, the active tamoxifen metabolite is selected from 4-hydroxytamoxifen (“4-OHT”), N-desmethyltamoxifen, tamoxifen-N-oxide, and endoxifen. In some embodiments, the non-endogenous ligand is administered to the subject at a concentration of between about 1 mg per day and about 100 mg per day. In particular embodiments, the non-endogenous ligand is administered to the subject at a concentration of about 40 mg per day. In some embodiments, the administering comprises administering one or more non-endogenous ligands to the human or animal. Exemplary non-endogenous ligands include, e.g., tamoxifen, 4-OHT, N-desmethyltamoxifen, tamoxifen-N-oxide, and endoxifen. In some embodiments, the administering comprises administering two or more non-endogenous ligands to the human or animal. In some embodiments, the two or more non-endogenous ligands include endoxifen and 4-OHT.

In some aspects, methods provided herein also include modulating transcription of a gene of interest in vivo, e.g., by delivering to a subject (i) a cell transformed with a chimeric transcription factor as described herein and (ii) a non-endogenous ligand. In some embodiments, the transformed cell comprises a target gene expression cassette comprising a chimeric-transcription factor responsive promoter operably linked the gene of interest.

In some embodiments, the subject is a human or animal, and contacting the transformed cell with the non-endogenous ligand comprises administering a pharmacological dose of the non-endogenous ligand to the human or animal.

In some aspects, methods provided herein also include delivering a composition in vivo capable of producing the engineered cells described herein, e.g., capable of delivering a polynucleotides described herein to a cell in vivo. Such compositions include any of the viral-mediated delivery platforms, any of the lipid structure delivery systems, any of the nanoparticle delivery systems, any of the genomic editing systems, or any of the other engineering delivery systems described herein capable of engineering a cell in vivo.

The methods provided herein also include delivering a composition in vivo capable of producing any of the modified ER-LBD, chimeric proteins, or chimeric transcription factors (and in some embodiments, a gene regulated by the chimeric transcription factor) as described herein. Compositions capable of in vivo production of the modified ER-LBD, chimeric protein, or chimeric transcription factor (and in some embodiments, a gene regulated by the chimeric transcription factor) include, but are not limited to, any of the engineered nucleic acids described herein. Compositions capable of in vivo production of inducible transcription factors (and in some embodiments, a gene regulated by the inducible transcription factor) can be a naked mRNA or a naked plasmid.

### Pharmaceutical Compositions

The modified ER-LBD, chimeric proteins, and cells of the present disclosure can be formulated in pharmaceutical compositions. These compositions can comprise, in addition to one or more of the engineered nucleic acids or engineered cells, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g., oral, intravenous, cutaneous or subcutaneous, nasal, intramuscular, intraperitoneal routes.

Whether it is a cell, polypeptide, nucleic acid, small molecule or other pharmaceutically useful compound according to the present disclosure that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of disease being treated. Prescription of treatment, e.g., decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### ENUMERATED EMBODIMENTS

Embodiment 1: An inducible cell-death system comprising a polypeptide, wherein the polypeptide comprises a ligand binding domain and a cell death inducing domain, wherein the polypeptide is configured upon contact with a ligand of the ligand binding domain to generate a cell-death inducing signal in a cell in which the polypeptide is expressed, and wherein
a. the ligand binding domain comprises a modified estrogen receptor ligand binding domain (ER-LBD) corresponding to a hormone binding domain of a reference human estrogen receptor sequence (SEQ ID NO: 1), wherein the modified ER-LBD comprises
   i. a G400V amino acid substitution, an M543A amino acid substitution, an L544A amino acid substitution, and optionally a V595A amino acid substitution, with reference to SEQ ID NO: 1; and
   ii. one or more additional amino acid substitutions, wherein the one or more additional amino acid substitutions are with reference to one or more regions selected from: positions 343-354, positions 380-392, positions 404-463, positions 517-540, and position 547 of SEQ ID NO: 1.

Embodiment 2: The inducible cell death system of Embodiment 1, wherein the polypeptide is or comprises a first polypeptide monomer and the inducible cell-death system further comprises a second polypeptide monomer, and
a. wherein the first polypeptide monomer and the second polypeptide monomer each comprise a ligand binding domain and a cell death inducing domain, wherein the first polypeptide monomer and the second polypeptide monomer are configured to oligomerize with each other upon contact with a ligand of the ligand binding domain, thereby generating the cell-death inducing signal in a cell in which the first polypeptide monomer and the second polypeptide monomer are expressed.

Embodiment 3: The inducible cell death system of Embodiment 2, wherein the ligand binding domain of the first polypeptide monomer and the second polypeptide monomer each comprise a modified ER-LBD, wherein the modified ER-LBD comprises:
i. a G400V amino acid substitution, an M543A amino acid substitution, an L544A amino acid substitution, and optionally a V595A amino acid substitution, with reference to SEQ ID NO: 1; and
ii. one or more additional amino acid substitutions, wherein the one or more additional amino acid substitutions are selected independently for each of the first polypeptide monomer and the second polypeptide monomer with reference to one or more regions selected from: positions 343-354, positions 380-392, positions 404-463, and positions 517-540, and position 547 of SEQ ID NO: 1.

Embodiment 4: The inducible cell death system of Embodiment 2 or 3, wherein the ligand binding domain of the first polypeptide monomer and the second polypeptide monomer comprise the same additional amino acid substitutions.

Embodiment 5: The inducible cell death system of any one of Embodiments 1 to 4, wherein the modified ER-LBD has greater sensitivity to a non-endogenous ligand as compared to an ER-LBD comprising the amino acid sequence of SEQ ID NO: 2.

Embodiment 6: The inducible cell death system of any one of Embodiments 1 to 5, wherein the modified ER-LBD has greater sensitivity to a non-endogenous ligand as compared to an endogenous ligand as a result of the one or more additional amino acid substitutions.

Embodiment 7: The inducible cell death system of any one of Embodiments 1 to 6, wherein the modified ER-LBD has greater selectivity to a non-endogenous ligand as compared to an ER-LBD comprising the amino acid sequence of SEQ ID NO: 2.

Embodiment 8: The inducible cell death system of any one of Embodiments 1 to 7, wherein the one or more additional amino acid substitutions of the polypeptide, the first polypeptide monomer, and/or the second polypeptide monomer comprise an L391 substitution.

Embodiment 9: The inducible cell death system of Embodiment 8, wherein the L391 substitution is L391V.

Embodiment 10: The inducible cell death system of any one of Embodiments 1 to 9, wherein the one or more additional amino acid substitutions of the polypeptide, the first polypeptide monomer, and/or the second polypeptide monomer comprise an N413D mutation.

Embodiment 11: The inducible cell death system of any one of Embodiments 1 to 7, wherein the one or more additional amino acid substitutions of the polypeptide, the first polypeptide monomer, and/or the second polypeptide monomer comprise an L391V substitution and an N413D mutation.

Embodiment 12: The inducible cell death system of any one of Embodiments 1 to 11, wherein the one or more additional amino acid substitutions of the polypeptide, the first polypeptide monomer, and/or the second polypeptide monomer comprises an H524 substitution.

Embodiment 13: The inducible cell death system of Embodiment 12, wherein the H524 substitution is an H524L substitution or an H524F substitution.

Embodiment 14: The inducible cell death system of Embodiment 12, wherein the H524 substitution is an H524L substitution.

Embodiment 15: The inducible cell death system of Embodiment 12, wherein the H524 substitution is an H524F substitution.

Embodiment 16: The inducible cell death system of any one of Embodiments 1 to 15, wherein the one or more additional amino acid substitutions of the polypeptide, the first polypeptide monomer, and/or the second polypeptide monomer comprises an M421L substitution.

Embodiment 17: The inducible cell death system of any one of Embodiments 1 to 16, wherein the one or more additional amino acid substitutions of the polypeptide, the first polypeptide monomer, and/or the second polypeptide monomer comprises an S463P substitution.

Embodiment 18: The inducible cell death system of any one of Embodiments 1 to 15, wherein the one or more additional amino acid substitutions of the polypeptide, the first polypeptide monomer, and/or the second polypeptide monomer comprises an M421L substitution and an S463P substitution.

Embodiment 19: The inducible cell death system of any one of Embodiments 1 to 18, wherein the one or more additional amino acid substitutions of the polypeptide, the first polypeptide monomer, and/or the second polypeptide monomer comprises an L384M substitution.

Embodiment 20: The inducible cell death system of any one of Embodiments 1 to 19, wherein the one or more additional amino acid substitutions of the polypeptide, the first polypeptide monomer, and/or the second polypeptide monomer comprises a L354I substitution.

Embodiment 21: The inducible cell death system of any one of Embodiments 1 to 20, wherein the one or more additional amino acid substitutions of the polypeptide, the first polypeptide monomer, and/or the second polypeptide monomer comprises a Q414E substitution.

Embodiment 22: The inducible cell death system of any one of Embodiments 1 to 19, wherein the one or more additional amino acid substitutions of the polypeptide, the first polypeptide monomer, and/or the second polypeptide monomer comprises a L354I substitution and a Q414E substitution.

Embodiment 23: The inducible cell death system of any one of Embodiments 1 to 22, wherein the one or more additional amino acid substitutions of the polypeptide, the first polypeptide monomer, and/or the second polypeptide monomer comprises an L391V substitution, an N413D mutation, and an H524 substitution.

Embodiment 24: The inducible cell death system of any one of Embodiments 1 to 22, wherein the one or more additional amino acid substitutions of the polypeptide, the first polypeptide monomer, and/or the second polypeptide monomer comprises an L391V substitution, an N413D mutation, an H524 substitution, and an M421L substitution.

Embodiment 25: The inducible cell death system of any one of Embodiments 1 to 22, wherein the one or more additional amino acid substitutions of the polypeptide, the first polypeptide monomer, and/or the second polypeptide monomer comprises an L391V substitution, an N413D mutation, an H524 substitution, and an S463P substitution.

Embodiment 26: The inducible cell death system of any one of Embodiments 1 to 22, wherein the one or more additional amino acid substitutions of the polypeptide, the first polypeptide monomer, and/or the second polypeptide monomer comprises an L391V substitution, an N413D mutation, an H524 substitution, and an Q414E substitution.

Embodiment 27: The inducible cell death system of any one of Embodiments 1 to 22, wherein the one or more additional amino acid substitutions of the polypeptide, the first polypeptide monomer, and/or the second polypeptide monomer comprises an L391V substitution, an N413D mutation, an H524 substitution, and an L354I substitution.

Embodiment 28: The inducible cell death system of any one of Embodiments 23 to 27, wherein the H524 substitution is an H524L substitution or an H524F substitution.

Embodiment 29: The inducible cell death system of any one of Embodiments 1 to 28, wherein the one or more additional amino acid substitutions of the polypeptide, the first polypeptide monomer, and/or the second polypeptide monomer are at one or more positions of SEQ ID NO: 1 selected from: 343, 344, 345, 346, 347, 348, 349, 350, 351, 352, 354, 380, 384, 386, 387, 388, 389, 391, 392, 404, 407, 409, 413, 414, 417, 418, 420, 421, 422, 424, 428, 463, 517, 521, 522, 524, 525, 526, 527, 528, 533, 534, 536, 537, 538, 539, 540, and 547.

Embodiment 30: The inducible cell death system of Embodiment 29, wherein the one or more positions comprise position 343 of SEQ ID NO: 1.

Embodiment 31: The inducible cell death system of Embodiment 30, wherein the amino acid substitution at position 343 of SEQ ID NO: 1 is selected from the group consisting of: M343F, M343I, M343L, and M343V.

Embodiment 32: The inducible cell death system of Embodiment 29, wherein the one or more positions comprise position 344 of SEQ ID NO: 1.

Embodiment 33: The inducible cell death system of Embodiment 32, wherein the amino acid substitution at position 344 of SEQ ID NO: 1 is G344M.

Embodiment 34: The inducible cell death system of Embodiment 29, wherein the one or more positions comprise position 345 of SEQ ID NO: 1.

Embodiment 35: The inducible cell death system of Embodiment 34, wherein the amino acid substitution at position 345 of SEQ ID NO: 1 is L345S.

Embodiment 36: The inducible cell death system of Embodiment 29, wherein the one or more positions comprise position 346 of SEQ ID NO: 1.

Embodiment 37: The inducible cell death system of Embodiment 36, wherein the amino acid substitution at position 346 of SEQ ID NO: 1 is selected from the group consisting of: L346I, L346M, L346F, and L346V.

Embodiment 38: The inducible cell death system of Embodiment 29, wherein the one or more positions comprise position 347 of SEQ ID NO: 1.

Embodiment 39: The inducible cell death system of Embodiment 38, wherein the amino acid substitution at position 347 of SEQ ID NO: 1 is selected from the group consisting of: T347D, T347E, T347F, T347I, T347K, T347L, T347M, T347N, T347Q, T347R, T347S, and T347V.

Embodiment 40: The inducible cell death system of Embodiment 29, wherein the one or more positions comprise position 348 of SEQ ID NO: 1.

Embodiment 41: The inducible cell death system of Embodiment 40, wherein the amino acid substitution at position 348 of SEQ ID NO: 1 is N348K.

Embodiment 42: The inducible cell death system of Embodiment 29, wherein the one or more positions comprise position 349 of SEQ ID NO: 1.

Embodiment 43: The inducible cell death system of Embodiment 42, wherein the amino acid substitution at position 349 of SEQ ID NO: 1 is selected from the group consisting of: L349I, L349M, L349F, and L349V.

Embodiment 44: The inducible cell death system of Embodiment 29, wherein the one or more positions comprise position 350 of SEQ ID NO: 1.

Embodiment 45: The inducible cell death system of Embodiment 44, wherein the amino acid substitution at position 350 of SEQ ID NO: 1 is selected from the group consisting of: A350F, A350I, A350L, A350M and A350V.

Embodiment 46: The inducible cell death system of Embodiment 29, wherein the one or more positions comprise position 351 of SEQ ID NO: 1.

Embodiment 47: The inducible cell death system of Embodiment 46, wherein the amino acid substitution at position 351 of SEQ ID NO: 1 is selected from the group consisting of: D351E, D351F, D351I, D351L, D351M, D351N, D351Q, and D351V.

Embodiment 48: The inducible cell death system of Embodiment 29, wherein the one or more positions comprise position 352 of SEQ ID NO: 1.

Embodiment 49: The inducible cell death system of Embodiment 48, wherein the amino acid substitution at position 352 of SEQ ID NO: 1 is R352K.

Embodiment 50: The inducible cell death system of Embodiment 29, wherein the one or more positions comprise position 354 of SEQ ID NO: 1.

Embodiment 51: The inducible cell death system of Embodiment 50, wherein the amino acid substitution at position 354 of SEQ ID NO: 1 is selected from the group consisting of: L354I, L354M, L354F, and L354V.

Embodiment 52: The inducible cell death system of Embodiment 29, wherein the one or more positions comprise position 380 of SEQ ID NO: 1.

Embodiment 53: The inducible cell death system of Embodiment 52, wherein the amino acid substitution at position 380 of SEQ ID NO: 1 is E380Q.

Embodiment 54: The inducible cell death system of Embodiment 29, wherein the one or more positions comprise position 384 of SEQ ID NO: 1.

Embodiment 55: The inducible cell death system of Embodiment 54, wherein the amino acid substitution at position 384 of SEQ ID NO: 1 is selected from the group consisting of: L384I, L384M, L384F, and L384V.

Embodiment 56: The inducible cell death system of Embodiment 29, wherein the one or more positions comprise position 386 of SEQ ID NO: 1.

Embodiment 57: The inducible cell death system of Embodiment 56, wherein the amino acid substitution at position 386 of SEQ ID NO: 1 is I386V.

Embodiment 58: The inducible cell death system of Embodiment 29, wherein the one or more positions comprise position 387 of SEQ ID NO: 1.

Embodiment 59: The inducible cell death system of Embodiment 58, wherein the amino acid substitution at position 387 of SEQ ID NO: 1 is selected from the group consisting of: L387I, L387M, L387F, and L387V.

Embodiment 60: The inducible cell death system of Embodiment 29, wherein the one or more positions comprise position 388 of SEQ ID NO: 1.

Embodiment 61: The inducible cell death system of Embodiment 60, wherein the amino acid substitution at position 388 of SEQ ID NO: 1 is selected from the group consisting of: M388I, M388L, and M388F.

Embodiment 62: The inducible cell death system of Embodiment 29, wherein the one or more positions comprise position 389 of SEQ ID NO: 1.

Embodiment 63: The inducible cell death system of Embodiment 62, wherein the amino acid substitution at position 389 of SEQ ID NO: 1 is I389M.

Embodiment 64: The inducible cell death system of Embodiment 29, wherein the one or more positions comprise position 391 of SEQ ID NO: 1.

Embodiment 65: The inducible cell death system of Embodiment 64, wherein the amino acid substitution at position 391 of SEQ ID NO: 1 is selected from the group consisting of: L391I, L391M, L391F, and L391V.

Embodiment 66: The inducible cell death system of Embodiment 64, wherein the amino acid substitution at position 391 of SEQ ID NO: 1 is L391V.

Embodiment 67: The inducible cell death system of Embodiment 29, wherein the one or more positions comprise position 392 of SEQ ID NO: 1.

Embodiment 68: The inducible cell death system of Embodiment 67, wherein the amino acid substitution at position 392 of SEQ ID NO: 1 is V392M.

Embodiment 69: The inducible cell death system of Embodiment 29, wherein the one or more positions comprise position 404 of SEQ ID NO: 1.

Embodiment 70: The inducible cell death system of Embodiment 69, wherein the amino acid substitution at position 404 of SEQ ID NO: 1 is selected from the group consisting of: F404I, F404L, F404M, and F404V.

Embodiment 71: The inducible cell death system of Embodiment 29, wherein the one or more positions comprise position 407 of SEQ ID NO: 1.

Embodiment 72: The inducible cell death system of Embodiment 71, wherein the amino acid substitution at position 407 of SEQ ID NO: 1 is N407D.

Embodiment 73: The inducible cell death system of Embodiment 29, wherein the one or more positions comprise position 409 of SEQ ID NO: 1.

Embodiment 74: The inducible cell death system of Embodiment 73, wherein the amino acid substitution at position 409 of SEQ ID NO: 1 is L409V.

Embodiment 75: The inducible cell death system of Embodiment 29, wherein the one or more positions comprise position 413 of SEQ ID NO: 1.

Embodiment 76: The inducible cell death system of Embodiment 75, wherein the amino acid substitution at position 413 of SEQ ID NO: 1 is N413D.

Embodiment 77: The inducible cell death system of Embodiment 29, wherein the one or more positions comprise position 414 of SEQ ID NO: 1.

Embodiment 78: The inducible cell death system of Embodiment 77, wherein the amino acid substitution at position 414 of SEQ ID NO: 1 is Q414E.

Embodiment 79: The inducible cell death system of Embodiment 29, wherein the one or more positions comprise position 417 of SEQ ID NO: 1.

Embodiment 80: The inducible cell death system of Embodiment 79, wherein the amino acid substitution at position 417 of SEQ ID NO: 1 is C417S.

Embodiment 81: The inducible cell death system of Embodiment 29, wherein the one or more positions comprise position 418 of SEQ ID NO: 1.

Embodiment 82: The inducible cell death system of Embodiment 81, wherein the amino acid substitution at position 418 of SEQ ID NO: 1 is selected from the group consisting of: V418I, V418L, V418M, and V418F.

Embodiment 83: The inducible cell death system of Embodiment 29, wherein the one or more positions comprise position 420 of SEQ ID NO: 1.

Embodiment 84: The inducible cell death system of Embodiment 83, wherein the amino acid substitution at position 420 of SEQ ID NO: 1 is selected from the group consisting of: G420I, G420M, G420F, and G420V.

Embodiment 85: The inducible cell death system of Embodiment 29, wherein the one or more positions comprise position 421 of SEQ ID NO: 1.

Embodiment 86: The inducible cell death system of Embodiment 85, wherein the amino acid substitution at position 421 of SEQ ID NO: 1 is selected from the group consisting of: M421I, M421L, M421F, and M421V.

Embodiment 87: The inducible cell death system of Embodiment 29, wherein the one or more positions comprise position 422 of SEQ ID NO: 1.

Embodiment 88: The inducible cell death system of Embodiment 87, wherein the amino acid substitution at position 422 of SEQ ID NO: 1 is V422I.

Embodiment 89: The inducible cell death system of Embodiment 29, wherein the one or more positions comprise position 424 of SEQ ID NO: 1.

Embodiment 90: The inducible cell death system of Embodiment 89, wherein the amino acid substitution at position 424 of SEQ ID NO: 1 is selected from the group consisting of: 1424L, 1424M, 1424F, and I424V.

Embodiment 91: The inducible cell death system of Embodiment 29, wherein the one or more positions comprise position 428 of SEQ ID NO: 1.

Embodiment 92: The inducible cell death system of Embodiment 91, wherein the amino acid substitution at position 428 of SEQ ID NO: 1 is selected from the group consisting of: L428I, LA28M, L428F, and L428V.

Embodiment 93: The inducible cell death system of Embodiment 29, wherein the one or more positions comprise position 463 of SEQ ID NO: 1.

Embodiment 94: The inducible cell death system of Embodiment 93, wherein the amino acid substitution at position 463 of SEQ ID NO: 1 is S463P.

Embodiment 95: The inducible cell death system of Embodiment 29, wherein the one or more positions comprise position 517 of SEQ ID NO: 1.

Embodiment 96: The inducible cell death system of Embodiment 95, wherein the amino acid substitution at position 517 of SEQ ID NO: 1 is M517A.

Embodiment 97: The inducible cell death system of Embodiment 29, wherein the one or more positions comprise position 521 of SEQ ID NO: 1.

Embodiment 98: The inducible cell death system of Embodiment 97, wherein the amino acid substitution at position 521 of SEQ ID NO: 1 is selected from the group consisting of: G521A, G521F, G521I, G521L, G521M, and G521V.

Embodiment 99: The inducible cell death system of Embodiment 29, wherein the one or more positions comprise position 522 of SEQ ID NO: 1.

Embodiment 100: The inducible cell death system of Embodiment 99, wherein the amino acid substitution at position 522 of SEQ ID NO: 1 is selected from the group consisting of: M522I, M522L, and M522V.

Embodiment 101: The inducible cell death system of Embodiment 29, wherein the one or more positions comprise position 524 of SEQ ID NO: 1.

Embodiment 102: The inducible cell death system of Embodiment 101, wherein the amino acid substitution at position 524 of SEQ ID NO: 1 is selected from the group consisting of: H524A, H524I, H524L, H524F, and H524V.

Embodiment 103: The inducible cell death system of Embodiment 29, wherein the one or more positions comprise position 525 of SEQ ID NO: 1.

Embodiment 104: The inducible cell death system of Embodiment 103, wherein the amino acid substitution at position 525 of SEQ ID NO: 1 is selected from the group consisting of: L525F, L525I, L525M, L525N, L525Q, L525S, L525T, and L525V.

Embodiment 105: The inducible cell death system of Embodiment 29, wherein the one or more positions comprise position 526 of SEQ ID NO: 1.

Embodiment 106: The inducible cell death system of Embodiment 105, wherein the amino acid substitution at position 526 of SEQ ID NO: 1 is Y526L.

Embodiment 107: The inducible cell death system of Embodiment 29, wherein the one or more positions comprise position 527 of SEQ ID NO: 1.

Embodiment 108: The inducible cell death system of Embodiment 107, wherein the amino acid substitution at position 527 of SEQ ID NO: 1 is S527N.

Embodiment 109: The inducible cell death system of Embodiment 29, wherein the one or more positions comprise position 528 of SEQ ID NO: 1.

Embodiment 110: The inducible cell death system of Embodiment 109, wherein the amino acid substitution at position 528 of SEQ ID NO: 1 is selected from the group consisting of: M528F, M528I, and M528V.

Embodiment 111: The inducible cell death system of Embodiment 29, wherein the one or more positions comprise position 533 of SEQ ID NO: 1.

Embodiment 112: The inducible cell death system of Embodiment 111, wherein the amino acid substitution at position 533 of SEQ ID NO: 1 is selected from the group consisting of: V533F and V533W.

Embodiment 113: The inducible cell death system of Embodiment 29, wherein the one or more positions comprise position 534 of SEQ ID NO: 1.

Embodiment 114: The inducible cell death system of Embodiment 113, wherein the amino acid substitution at position 534 of SEQ ID NO: 1 is selected from the group consisting of: V534Q and V534R.

Embodiment 115: The inducible cell death system of Embodiment 29, wherein the one or more positions comprise position 536 of SEQ ID NO: 1.

Embodiment 116: The inducible cell death system of Embodiment 115, wherein the amino acid substitution at position 536 of SEQ ID NO: 1 is selected from the group consisting of: L536F, and L536M, L536R, and L536Y.

Embodiment 117: The inducible cell death system of Embodiment 29, wherein the one or more positions comprise position 537 of SEQ ID NO: 1.

Embodiment 118: The inducible cell death system of Embodiment 117, wherein the amino acid substitution at position 537 of SEQ ID NO: 1 is selected from the group consisting of: Y537E and Y537S.

Embodiment 119: The inducible cell death system of Embodiment 29, wherein the one or more positions comprise position 538 of SEQ ID NO: 1.

Embodiment 120: The inducible cell death system of Embodiment 119, wherein the amino acid substitution at position 538 of SEQ ID NO: 1 is selected from the group consisting of: D538G and D538K.

Embodiment 121: The inducible cell death system of Embodiment 29, wherein the one or more positions comprise position 539 of SEQ ID NO: 1.

Embodiment 122: The inducible cell death system of Embodiment 121, wherein the amino acid substitution at position 539 of SEQ ID NO: 1 is selected from the group consisting of: L539A and L539R.

Embodiment 123: The inducible cell death system of Embodiment 29, wherein the one or more positions comprise position 540 of SEQ ID NO: 1.

Embodiment 124: The inducible cell death system of Embodiment 123, wherein the amino acid substitution at position 540 of SEQ ID NO: 1 is selected from the group consisting of: L540A and L540F.

Embodiment 125: The inducible cell death system of Embodiment 29, wherein the one or more positions comprise position 547 of SEQ ID NO: 1.

Embodiment 126: The inducible cell death system of Embodiment 125, wherein the amino acid substitution at position 547 of SEQ ID NO: 1 is H547A.

Embodiment 127: The inducible cell death system of any one of Embodiments 1-126, wherein the one or more additional amino acid substitutions of the polypeptide, the first polypeptide monomer, and/or the second polypeptide monomer are two amino acid substitutions.

Embodiment 128: The inducible cell death system of Embodiment 127, wherein each of the two amino acid substitutions are at a position of SEQ ID NO: 1 selected from: 343, 345, 347, 348, 351, 354, 384, 387, 388, 389, 391, 392, 404, 418, 421, 521, 524, and 525.

Embodiment 129: The inducible cell death system of Embodiment 128, wherein the two amino acid substitutions are at positions 345 and 348 of SEQ ID NO: 1.

Embodiment 130: The inducible cell death system of Embodiment 129, wherein the amino acid substitution at position 345 of SEQ ID NO: 1 is L345S and the amino acid substitution at position 348 of SEQ ID NO: 1 is N348K.

Embodiment 131: The inducible cell death system of Embodiment 128, wherein the two amino acid substitutions are at positions 384 and 389 of SEQ ID NO: 1.

Embodiment 132: The inducible cell death system of Embodiment 131, wherein the amino acid substitution at position 384 of SEQ ID NO: 1 is L384M and the amino acid substitution at position 389 of SEQ ID NO: 1 is I389M.

Embodiment 133: The inducible cell death system of Embodiment 128, wherein the two amino acid substitutions are at positions 421 and 392 of SEQ ID NO: 1.

Embodiment 134: The inducible cell death system of Embodiment 133, wherein the amino acid substitution at position 421 of SEQ ID NO: 1 is M421I and the amino acid substitution at position 392 of SEQ ID NO: 1 is V392M.

Embodiment 135: The inducible cell death system of Embodiment 128, wherein the two amino acid substitutions are at positions 354 and 391 of SEQ ID NO: 1.

Embodiment 136: The inducible cell death system of Embodiment 135, wherein the amino acid substitution at position 354 of SEQ ID NO: 1 is L354I and the amino acid substitution at position 391 of SEQ ID NO: 1 is L391F.

Embodiment 137: The inducible cell death system of Embodiment 128, wherein the two amino acid substitutions are at positions 354 and 384 of SEQ ID NO: 1.

Embodiment 138: The inducible cell death system of Embodiment 137, wherein the amino acid substitution at position 354 of SEQ ID NO: 1 is L354I and the amino acid substitution at position 384 of SEQ ID NO: 1 is L384M.

Embodiment 139: The inducible cell death system of Embodiment 128, wherein the two amino acid substitutions are at positions 354 and 387 of SEQ ID NO: 1.

Embodiment 140: The inducible cell death system of Embodiment 139, wherein the amino acid substitution at position 354 of SEQ ID NO: 1 is L354I and the amino acid substitution at position 387 of SEQ ID NO: 1 is L387M.

Embodiment 141: The inducible cell death system of Embodiment 128, wherein the two amino acid substitutions are at positions 387 and 391.

Embodiment 142: The inducible cell death system of Embodiment 141, wherein the amino acid substitution at position 387 of SEQ ID NO: 1 is L387M and the amino acid substitution at position 391 of SEQ ID NO: 1 is L391F.

Embodiment 143: The inducible cell death system of Embodiment 128, wherein the two amino acid substitutions are at positions 384 and 387 of SEQ ID NO: 1.

Embodiment 144: The inducible cell death system of Embodiment 143, wherein the amino acid substitution at position 384 of SEQ ID NO: 1 is L384M and the amino acid substitution at position 387 of SEQ ID NO: 1 is L387M.

Embodiment 145: The inducible cell death system of Embodiment 128, wherein the two amino acid substitutions are at positions 384 and 391 of SEQ ID NO: 1.

Embodiment 146: The inducible cell death system of Embodiment 145, wherein the amino acid substitution at position 384 of SEQ ID NO: 1 is L384M and the amino acid substitution at position 391 of SEQ ID NO: 1 is L391F.

Embodiment 147: The inducible cell death system of any one of Embodiments 1 to 146, wherein the one or more additional amino acid substitutions of the polypeptide, the first polypeptide monomer, and/or the second polypeptide monomer are three amino acid substitutions.

Embodiment 148: The inducible cell death system of Embodiment 147, wherein each of the three amino acid substitutions are at a position of SEQ ID NO: 1 selected from: 343, 347, 351, 354, 388, 391, 404, 414, 418, 463, 521, 524, and 525.

Embodiment 149: The inducible cell death system of Embodiment 148, wherein the three amino acid substitutions are at positions 354, 384, and 391 of SEQ ID NO: 1.

Embodiment 150: The inducible cell death system of Embodiment 149, wherein the amino acid substitution at position 354 of SEQ ID NO: 1 is L354I, the amino acid substitution at position 384 of SEQ ID NO: 1 is L384M, and the amino acid substitution at position 391 of SEQ ID NO: 1 is L391F.

Embodiment 151: The inducible cell death system of Embodiment 148, wherein the three amino acid substitutions are at positions 414, 463, and 524 of SEQ ID NO: 1.

Embodiment 152: The inducible cell death system of Embodiment 151, wherein the amino acid substitution at position 414 of SEQ ID NO: 1 is Q414E, the amino acid substitution at position 463 of SEQ ID NO: 1 is S463P, and the amino acid substitution at position 524 of SEQ ID NO: 1 is H524L.

Embodiment 153: The inducible cell death system of any one of Embodiments 1 to 152, wherein the one or more additional amino acid substitutions of the polypeptide, the first polypeptide monomer, and/or the second polypeptide monomer are four amino acid substitutions.

Embodiment 154: The inducible cell death system of Embodiment 153, wherein each of the four amino acid substitutions are at a position of SEQ ID NO: 1 selected from: 343, 347, 351, 354, 384, 388, 391, 404, 413, 418, 463, 521, 524, and 525.

Embodiment 155: The inducible cell death system of Embodiment 154, wherein the four amino acid substitutions are at positions 354, 384, 391, and 418 of SEQ ID NO: 1.

Embodiment 156: The inducible cell death system of Embodiment 155, wherein the amino acid substitution at position 354 of SEQ ID NO: 1 is L354I, the amino acid substitution at position 384 of SEQ ID NO: 1 is L384M, the amino acid substitution at position 391 of SEQ ID NO: 1 is L391F, and the amino acid substitution at position 418 of SEQ ID NO: 1 is V418I.

Embodiment 157: The inducible cell death system of Embodiment 154, wherein the four amino acid substitutions are at positions 343, 388, 521, and 404 of SEQ ID NO: 1.

Embodiment 158: The inducible cell death system of Embodiment 157, wherein the amino acid substitution at position 343 of SEQ ID NO: 1 is M343I, the amino acid substitution at position 388 of SEQ ID NO: 1 is M388I, the amino acid substitution at position 521 of SEQ ID NO: 1 is G521I, and the amino acid substitution at position 404 of SEQ ID NO: 1 is F404L.

Embodiment 159: The inducible cell death system of Embodiment 154, wherein the four amino acid substitutions are at positions 524, 347, 351, and 525 of SEQ ID NO: 1.

Embodiment 160: The inducible cell death system of Embodiment 159, wherein the amino acid substitution at position 524 of SEQ ID NO: 1 is H524V, the amino acid substitution at position 347 of SEQ ID NO: 1 is T347R, the amino acid substitution at position 351 of SEQ ID NO: 1 is D351Q, and the amino acid substitution at position 525 of SEQ ID NO: 1 is L525N.

Embodiment 161: The inducible cell death system of Embodiment 154, wherein the four amino acid substitutions are at positions 354, 384, 391, and 463 of SEQ ID NO: 1.

Embodiment 162: The inducible cell death system of Embodiment 161, wherein the amino acid substitution at position 354 of SEQ ID NO: 1 is L354I, the amino acid substitution at position 384 of SEQ ID NO: 1 is L384M, the amino acid substitution at position 391 of SEQ ID NO: 1 is L391V, and the amino acid substitution at position 463 of SEQ ID NO: 1 is S463P.

Embodiment 163: The inducible cell death system of Embodiment 154, wherein the four amino acid substitutions are at positions 384, 391, 413, and 524 of SEQ ID NO: 1.

Embodiment 164: The inducible cell death system of Embodiment 163, wherein the amino acid substitution at position 384 of SEQ ID NO: 1 is L384M, the amino acid substitution at position 391 of SEQ ID NO: 1 is L391V, the amino acid substitution at position 413 of SEQ ID NO: 1 is N413D, and the amino acid substitution at position 524 of SEQ ID NO: 1 is H524F.

Embodiment 165: The inducible cell death system of any one of Embodiments 1 to 164, wherein the one or more additional amino acid substitutions of the polypeptide, the first polypeptide monomer, and/or the second polypeptide monomer are five amino acid substitutions.

Embodiment 166: The inducible cell death system of Embodiment 165, wherein each of the five amino acid substitutions are at a position of SEQ ID NO: 1 selected from: 354, 384, 391, 409, 413, 414, 421, 463, and 524.

Embodiment 167: The inducible cell death system of Embodiment 166, wherein the five amino acid substitutions are at positions 384, 409, 413, 463, and 524 of SEQ ID NO: 1.

Embodiment 168: The inducible cell death system of Embodiment 167, wherein the amino acid substitution at position 384 of SEQ ID NO: 1 is L384M, the amino acid substitution at position 409 of SEQ ID NO: 1 is L409V, the amino acid substitution at position 413 of SEQ ID NO: 1 is N413D, the amino acid substitution at position 463 of SEQ ID NO: 1 is S463P, and the amino acid substitution at position 524 of SEQ ID NO: 1 is H524L.

Embodiment 169: The inducible cell death system of Embodiment 166, wherein the five amino acid substitutions are at positions 391, 413, 414, 463, and 524 of SEQ ID NO: 1.

Embodiment 170: The inducible cell death system of Embodiment 169, wherein the amino acid substitution at position 391 of SEQ ID NO: 1 is L391V, the amino acid substitution at position 413 of SEQ ID NO: 1 is N413D, the amino acid substitution at position 414 of SEQ ID NO: 1 is Q414E, the amino acid substitution at position 463 of SEQ ID NO: 1 is S463P, and the amino acid substitution at position 524 of SEQ ID NO: 1 is H524F.

Embodiment 171: The inducible cell death system of embodiment 166, wherein the five amino acid substitutions are at positions 391, 414, 421, 463, and 524 of SEQ ID NO: 1.

Embodiment 172: The inducible cell death system of Embodiment 171, wherein the amino acid substitution at position 391 of SEQ ID NO: 1 is L391V, the amino acid substitution at position 414 of SEQ ID NO: 1 is Q414E, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524F.

Embodiment 173: The inducible cell death system of Embodiment 166, wherein the five amino acid substitutions are at positions 354, 409, 413, 421, and 524 of SEQ ID NO: 1.

Embodiment 174: The inducible cell death system of Embodiment 173, wherein the amino acid substitution at position 354 of SEQ ID NO: 1 is L354I, the amino acid substitution at position 409 of SEQ ID NO: 1 is L409V, the amino acid substitution at position 413 of SEQ ID NO: 1 is N413D, the amino acid substitution at position 421 of SEQ ID NO: 1 is M421L, and the amino acid substitution at position 524 of SEQ ID NO: 1 is H524L.

Embodiment 175: The inducible cell death system of Embodiment 166, wherein the five amino acid substitutions are at positions 354, 409, 421, 463, and 524 of SEQ ID NO: 1.

Embodiment 176: The inducible cell death system of Embodiment 175, wherein the amino acid substitution at position 354 of SEQ ID NO: 1 is L354I, the amino acid substitution at position 409 of SEQ ID NO: 1 is L409V, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524L.

Embodiment 177: The inducible cell death system of any one of Embodiments 1-176, wherein the one or more additional amino acid substitutions of the polypeptide, the first polypeptide monomer, and/or the second polypeptide monomer are six amino acid substitutions.

Embodiment 178: The inducible cell death system of Embodiment 177, wherein each of the six amino acid substitutions are at a position of SEQ ID NO: 1 selected from: 354, 384, 391, 409, 413, 414, 421, 463, and 524.

Embodiment 179: The inducible cell death system of Embodiment 178, wherein the six amino acid substitutions are at positions 384, 391, 413, 421, 463, and 524 of SEQ ID NO: 1.

Embodiment 180: The inducible cell death system of Embodiment 179, wherein the amino acid substitution at position 384 of SEQ ID NO: 1 is L384M, the amino acid substitution at position 391 of SEQ ID NO: 1 is L391V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524L.

Embodiment 181: The inducible cell death system of Embodiment 178, wherein the six amino acid substitutions are at positions 409, 413, 414, 421, 463, and 524 of SEQ ID NO: 1.

Embodiment 182: The inducible cell death system of Embodiment 181, wherein the amino acid substitution at position 409 of SEQ ID NO: 1 is L409V, the amino acid substitution at position 413 of SEQ ID NO: 1 is N413D, the amino acid substitution at position 414 of SEQ ID NO: 1 is Q414E, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524L.

Embodiment 183: The inducible cell death system of Embodiment 178, wherein the six amino acid substitutions are at positions 354, 391, 409, 413, 414, and 524 of SEQ ID NO: 1.

Embodiment 184: The inducible cell death system of Embodiment 183, wherein the amino acid substitution at position 354 of SEQ ID NO: 1 is L354I, the amino acid substitution at position 391 of SEQ ID NO: 1 is L391V, the amino acid substitution at position 409 of SEQ ID NO: 1 is L409V, the amino acid substitution at position 413 of SEQ ID NO: 1 is N413D, the amino acid substitution at position 414 of SEQ ID NO: 1 is Q414E, and the amino acid substitution at position 524 of SEQ ID NO: 1 is H524L.

Embodiment 185: The inducible cell death system of any one of the preceding Embodiments, wherein the one or more additional amino acid substitutions of the polypeptide, the first polypeptide monomer, and/or the second polypeptide monomer are seven amino acid substitutions.

Embodiment 186: The inducible cell death system of Embodiment 185, wherein each of the seven amino acid substitutions are at a position of SEQ ID NO: 1 selected from: 354, 384, 391, 409, 413, 414, 421, 463, 517, and 524.

Embodiment 187: The inducible cell death system of Embodiment 186, wherein the seven amino acid substitutions are at positions 354, 384, 409, 413, 421, 463, and 524 of SEQ ID NO: 1.

Embodiment 188: The inducible cell death system of Embodiment 187, wherein the amino acid substitution at position 354 of SEQ ID NO: 1 is L354I, the amino acid substitution at position 384 of SEQ ID NO: 1 is L384M, the amino acid substitution at position 409 of SEQ ID NO: 1 is L409V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524F.

Embodiment 189: The inducible cell death system of Embodiment 186, wherein the seven amino acid substitutions are at positions 354, 391, 413, 421, 463, 517, and 524 of SEQ ID NO: 1.

Embodiment 190: The inducible cell death system of Embodiment 189, wherein the amino acid substitution at position 354 of SEQ ID NO: 1 is L354I, the amino acid substitution at position 391 of SEQ ID NO: 1 is L391V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, the amino acid substitution at position 517 of SEQ ID NO: 1 is M517A, and the amino acid substitution at position 524 of SEQ ID NO: 1 is H524L.

Embodiment 191: The inducible cell death system of Embodiment 186, wherein the seven amino acid substitutions are at positions 354, 391, 413, 414, 421, 517, and 524 of SEQ ID NO: 1.

Embodiment 192: The inducible cell death system of Embodiment 191, wherein the amino acid substitution at position 354 of SEQ ID NO: 1 is L354I, the amino acid substitution at position 391 of SEQ ID NO: 1 is L391V, the amino acid substitution at position 413 of SEQ ID NO: 1 is N413D, the amino acid substitution at position 414 of SEQ ID NO: 1 is Q414E, the amino acid substitution at position 421 of SEQ ID NO: 1 is M421L, the amino acid substitution at position 517 of SEQ ID NO: 1 is M517A, and the amino acid substitution at position 524 of SEQ ID NO: 1 is H524F.

Embodiment 193: The inducible cell death system of any one of the preceding Embodiments, wherein the one or more additional amino acid substitutions of the polypeptide, the first polypeptide monomer, and/or the second polypeptide monomer are eight amino acid substitutions.

Embodiment 194: The inducible cell death system of Embodiment 193, wherein the eight amino acid substitutions are at positions 384, 391, 409, 413, 421, 463, 517, and 524 of SEQ ID NO: 1.

Embodiment 195: The inducible cell death system of Embodiment 194, wherein the amino acid substitution at position 384 of SEQ ID NO: 1 is L384M, the amino acid substitution at position 391 of SEQ ID NO: 1 is L391V, the amino acid substitution at position 409 of SEQ ID NO: 1 is L409V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, the amino acid substitution at position 517 of SEQ ID NO: 1 is M517A, and the amino acid substitution at position 524 of SEQ ID NO: 1 is H524F.

Embodiment 196: The inducible cell death system of any one of the preceding Embodiments, wherein the modified ER-LBD further comprises the V595A amino acid substitution.

Embodiment 197: An inducible cell death system comprising a first polypeptide and a second polypeptide monomer, wherein the first and the second polypeptide monomers each comprise a ligand binding domain and a cell death inducing domain, wherein the first and the second polypeptide monomers are configured to oligomerize upon contact with a ligand of the ligand binding domain, thereby generating a cell-death inducing signal in a cell in which the first and the second polypeptide monomers are expressed, and wherein
a. the ligand binding domain comprises a modified estrogen receptor ligand binding domain (ER-LBD) comprising an amino acid sequence corresponding to a hormone binding domain of a reference human estrogen receptor sequence (SEQ ID NO: 1), wherein the modified ER-LBD comprises:
   i. a G400V amino acid substitution, an M543A amino acid substitution, an L544A amino acid substitution, and optionally a V595A amino acid substitution, with reference to SEQ ID NO: 1; and
   ii. additional amino acid substitutions, wherein the additional amino acid substitutions comprise, with reference to SEQ ID NO: 1:
      a) an L384M substitution, an L391V substitution, a N413D substitution, an M421L substitution, a S463P substitution, and a H524L substitution,
      b) an L391V substitution, a N413D substitution, a Q414E substitution, a S463P substitution, and a H524F substitution,
      c) an L354I substitution, a L391V substitution, a N413D substitution, a Q414E substitution, a M421L substitution, a M517A substitution, and a H524F substitution, or
      d) an L354I substitution, a L391V substitution, a LA09V substitution, a N413D substitution, a Q414E substitution, and a H524L substitution.

Embodiment 198: The inducible cell death system of any one of Embodiments 1-6, wherein the one or more additional amino acid substitutions of the polypeptide, the first polypeptide monomer, and/or the second polypeptide monomer comprises an N413D mutation, an H524 substitution, and an S463P substitution.

Embodiment 199: The inducible cell death system of Embodiment 198, wherein the one or more additional amino acid substitutions of the polypeptide, the first polypeptide monomer, and/or the second polypeptide monomer comprise an L391V substitution, an L409V substitution, an Q414E substitution, an N413D substitution, an S463P substitution, an M517A substitution, and an H524L substitution.

Embodiment 200: The inducible cell death system of Embodiment 199, wherein: the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 90 or 103.

Embodiment 201: The inducible cell death system of Embodiment 198, wherein the one or more additional amino acid substitutions of the polypeptide, the first polypeptide monomer, and/or the second polypeptide monomer comprise an LA09V substitution, an N413D substitution, an S463P substitution, an M421L substitution, an L384M substitution, and an H524L substitution.

Embodiment 202: The inducible cell death system of Embodiment 201, wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 91 or 104.

Embodiment 203: The inducible cell death system of Embodiment 198, wherein the one or more additional amino acid substitutions of the polypeptide, the first polypeptide monomer, and/or the second polypeptide monomer comprise an L391V substitution, an L409V substitution, an N413D substitution, an S463P substitution, an M517A substitution, an M421L substitution, an L354I substitution, and an H524L substitution.

Embodiment 204: The inducible cell death system of Embodiment 203, wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 92 or 105.

Embodiment 205: The inducible cell death system of Embodiment 198, wherein the one or more additional amino acid substitutions of the polypeptide, the first polypeptide monomer, and/or the second polypeptide monomer comprise an L391V substitution, an Q414E substitution, an N413D substitution, an S463P substitution, an M421L substitution, an L354I substitution, an L384M substitution, and an H524L substitution.

Embodiment 206: The inducible cell death system of Embodiment 205, wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 93 or 106.

Embodiment 207: The inducible cell death system of Embodiment 198, wherein the one or more additional amino acid substitutions of the polypeptide, the first polypeptide monomer, and/or the second polypeptide monomer comprise an L391V substitution, an L409V substitution, an N413D substitution, an S463P substitution, an M517A substitution, an M421L substitution, and an H524L substitution.

Embodiment 208: The inducible cell death system of Embodiment 207, wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 94 or 107.

Embodiment 209: The inducible cell death system of Embodiment 198, wherein the one or more additional amino acid substitutions of the polypeptide, the first polypeptide monomer, and/or the second polypeptide monomer comprise an L391V substitution, an L409V substitution, an Q414E substitution, an N413D substitution, an S463P substitution, an L354I substitution, and an H524L substitution.

Embodiment 210: The inducible cell death system of Embodiment 209, wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 95 or 108.

Embodiment 211: The inducible cell death system of Embodiment 198, wherein the one or more additional amino acid substitutions of the polypeptide, the first polypeptide monomer, and/or the second polypeptide monomer comprise an L391V substitution, an LA09V substitution, an N413D substitution, an S463P substitution, an M421L substitution, an L354I substitution, an L384M substitution, and an H524L substitution.

Embodiment 212: The inducible cell death system of Embodiment 211, wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 96 or 109.

Embodiment 213: The inducible cell death system of Embodiment 198, wherein the one or more additional amino acid substitutions of the polypeptide, the first polypeptide monomer, and/or the second polypeptide monomer comprise an L391V substitution, an Q414E substitution, an N413D substitution, an S463P substitution, an M517A substitution, an M421L substitution, an L354I substitution, and an H524L substitution.

Embodiment 214: The inducible cell death system of Embodiment 213, wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 97 or 110.

Embodiment 215: The inducible cell death system of Embodiment 198, wherein the one or more additional amino acid substitutions of the polypeptide, the first polypeptide monomer, and/or the second polypeptide monomer comprise an L391V substitution, an N413D substitution, an S463P substitution, an M517A substitution, an L384M substitution, and an H524L substitution.

Embodiment 216: The inducible cell death system of Embodiment 215, wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 98 or 111.

Embodiment 217: The inducible cell death system of Embodiment 198, wherein the one or more additional amino acid substitutions of the polypeptide, the first polypeptide monomer, and/or the second polypeptide monomer comprise an L391V substitution, an L409V substitution, an N413D substitution, an S463P substitution, an M517A substitution, and an H524L substitution.

Embodiment 218: The inducible cell death system of Embodiment 217, wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 99 or 112.

Embodiment 219: The inducible cell death system of Embodiment 198, wherein the one or more additional amino acid substitutions of the polypeptide, the first polypeptide monomer, and/or the second polypeptide monomer comprise an N413D substitution, an S463P substitution, an L354I substitution, an L384M substitution, and an H524L substitution

Embodiment 220: The inducible cell death system of Embodiment 219, wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 100 or 113.

Embodiment 221: The inducible cell death system of Embodiment 198, wherein the one or more additional amino acid substitutions of the polypeptide, the first polypeptide monomer, and/or the second polypeptide monomer comprise an N413D substitution, an S463P substitution, an M421L substitution, an L354I substitution, and an H524L substitution

Embodiment 222: The inducible cell death system of Embodiment 221, wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 101 or 114.

Embodiment 223: The inducible cell death system of any one of the above Embodiments, wherein the ligand is a non-endogenous ligand.

Embodiment 224: The inducible cell death system of Embodiment 223, wherein the non-endogenous ligand is selected from: 4-hydroxytamoxifen, N-desmethyltamoxifen, tamoxifen-N-oxide, and endoxifen.

Embodiment 225: The inducible cell death system of Embodiment 224, wherein the non-endogenous ligand comprises a tamoxifen metabolite.

Embodiment 226: The inducible cell death system of Embodiment 224 or 225, wherein the non-endogenous ligand is endoxifen.

Embodiment 227: The inducible cell death system of any one of Embodiments 224-226, wherein the first polypeptide monomer and the second polypeptide monomer are capable of oligomerization and/or generating the cell-death inducing signal at a concentration of 0.25 nM Endoxifen or less and/or at a concentration of 0.04 nM 4-OHT or less.

Embodiment 228: The inducible cell death system of any one of Embodiments 224-227, wherein the first polypeptide monomer and the second polypeptide monomer are capable of oligomerization and/or generating the cell-death inducing signal at a concentration of 2.5 nM Endoxifen or less and/or at a concentration of 0.4 nM 4-OHT or less.

Embodiment 229: The inducible cell death system of any one of Embodiments 224-228, wherein the first polypeptide monomer and the second polypeptide monomer are capable of oligomerization and/or generating the cell-death inducing signal at a concentration of at least 0.001 pM of 4-OHT.

Embodiment 230: The inducible cell death system of any one of Embodiments 224-229, wherein the first polypeptide monomer and the second polypeptide monomer are capable of oligomerization and/or generating the cell-death inducing signal at a concentration of at least 0.01 pM of 4-OHT.

Embodiment 231: The inducible cell death system of any one of the above Embodiments, wherein the cell death-inducing domain is derived from a protein selected from: caspase 3, caspase 6, caspase 7, caspase 8, caspase 9, Diphtheria toxin fragment A (DTA), Bax, Bak, Bok, Bad, Bcl-Xs, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B1, and Purine nucleoside phosphorylase.

Embodiment 232: The inducible cell death system of any one of the above Embodiments, wherein the cell death-inducing domain comprises a caspase domain or a functional fragment thereof.

Embodiment 233: The inducible cell death system of Embodiment 232, wherein the caspase is selected from: caspase 3, caspase 6, caspase 7, caspase 8, caspase 9, or functional fragments thereof, respectively.

Embodiment 234: The inducible cell death system of Embodiment 233, wherein the caspase is caspase 9, or a functional fragment thereof.

Embodiment 235: The inducible cell death system of Embodiment 234, wherein the cell death-inducing domain comprises the Caspase 9 derived amino acid sequence of SEQ ID NO:48 or 125.

Embodiment 236: The inducible cell death system of any one of Embodiments 232-235, wherein the caspase domain or functional fragment thereof does not comprise a Caspase Activation and Recruitment Domain (CARD) domain sequence.

Embodiment 237: The inducible cell death system of any one of the above Embodiments, wherein the cell death-inducing domain is a transcription factor comprising a nucleic acid-binding domain and a transcriptional effector domain, wherein the transcription factor is configured to generate a cell-death inducing signal by inducing expression of: a caspase domain or functional fragment thereof, optionally wherein the caspase is selected from caspase 3, caspase 6, caspase 7, caspase 8, caspase 9, or functional fragments thereof, respectively, Diphtheria toxin fragment A (DTA), Bax, Bak, Bok, Bad, Bcl-Xs, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B1, or Purine nucleoside phosphorylase.

Embodiment 238: An isolated polynucleotide comprising a nucleotide sequence encoding the polypeptide, the first polypeptide monomer, and/or the second polypeptide monomer of any one of Embodiments 1-237.

Embodiment 239: A heterologous construct comprising a promoter operatively linked to the polynucleotide of Embodiment 238.

Embodiment 240: A plasmid or a vector comprising the heterologous construct of Embodiment 239.

Embodiment 241: A cell comprising the heterologous construct of Embodiment 239 or the plasmid or the vector of Embodiment 240.

Embodiment 242: A molecular switch for generating a cell-death inducing signal in a cell, comprising:
i. the inducible cell death system of any one of Embodiments 1-237, the isolated polynucleotide of Embodiment 238, the heterologous construct of Embodiment 239, the plasmid or vector of Embodiment 240, or the cell of Embodiment 241, wherein the inducible cell death system is capable of generating a cell-death inducing signal in the cell; and
ii. a non-endogenous ligand, wherein binding of the non-endogenous ligand to the modified ER-LBD generates the cell-death inducing signal in the cell.

Embodiment 243: A molecular switch for generating a cell-death inducing signal in a cell, comprising:
i. the inducible cell death system of any one of Embodiments 2-237, the isolated polynucleotide of Embodiment 238, the heterologous construct of Embodiment 239, the plasmid or vector of Embodiment 240, or the cell of Embodiment 241, wherein the inducible cell death system is capable of generating a cell-death inducing signal in the cell upon oligomerization of the first and the second polypeptide monomers; and
ii. a non-endogenous ligand, wherein binding of the non-endogenous ligand to the modified ER-LBD induces oligomerization of the first and the second polypeptide monomers, thereby generating the cell-death inducing signal in the cell.

Embodiment 244: The molecular switch of Embodiment 242 or 243, wherein the non-endogenous ligand is selected from: 4-hydroxytamoxifen, N-desmethyltamoxifen, tamoxifen-N-oxide, and endoxifen.

Embodiment 245: The molecular switch of any one of Embodiments 242-244, wherein the non-endogenous ligand comprises a tamoxifen metabolite.

Embodiment 246: The molecular switch of any one of Embodiments 242-245, wherein the non-endogenous ligand is endoxifen.

Embodiment 247: The molecular switch of any one of Embodiments 244-246, wherein the first polypeptide monomer and the second polypeptide monomer are capable of oligomerization and/or generating the cell-death inducing signal at a concentration of 0.25 nM Endoxifen or less and/or at a concentration of 0.04 nM 4-OHT or less.

Embodiment 248: The molecular switch of any one of Embodiments 244-247, wherein the first polypeptide monomer and the second polypeptide monomer are capable of oligomerization and/or generating the cell-death inducing signal at a concentration of 2.5 nM Endoxifen or less and/or at a concentration of 0.4 nM 4-OHT or less.

Embodiment 249: The molecular switch of any one of Embodiments 244-248, wherein the first polypeptide monomer and the second polypeptide monomer are capable of oligomerization and/or generating the cell-death inducing signal at a concentration of at least 0.001 pM of 4-OHT.

Embodiment 250: The molecular switch of any one of Embodiments 244-249, wherein the first polypeptide monomer and the second polypeptide monomer are capable of oligomerization and/or generating the cell-death inducing signal at a concentration of at least 0.01 pM of 4-OHT.

Embodiment 251: A method of inducing cell death, comprising: transforming a cell with (i) a heterologous construct encoding any one of the above inducible cell death systems and (ii) contacting the transformed cell with a non-endogenous ligand of the modified estrogen receptor ligand binding domain (ER-LBD).

Embodiment 252: A method of inducing oligomerization of a chimeric protein comprising: transforming a cell with (i) a heterologous construct encoding any one of the inducible cell death systems of any one of Embodiments 2-237, the isolated polynucleotide of Embodiment 238, the heterologous construct of Embodiment 239, or the plasmid or vector of Embodiment 240, and (ii) contacting the transformed cell with a non-endogenous ligand of the modified estrogen receptor ligand binding domain (ER-LBD).

Embodiment 253: The method of Embodiment 252, the method further comprising culturing the transformed cell under conditions suitable for expression of the of the inducible cell death system prior to inducing oligomerization and/or inducing cell death.

Embodiment 254: The method of any one of Embodiments 252-253, wherein the transformed cell is in a human or animal, and wherein contacting the transformed cell with the non-endogenous ligand comprises administering a pharmacological dose of the ligand to the human or animal.

Embodiment 255: The method of any one of Embodiments 252-254, wherein the non-endogenous ligand is selected from: 4-hydroxytamoxifen, N-desmethyltamoxifen, tamoxifen-N-oxide, and endoxifen.

Embodiment 256: The method of any one of Embodiments 252-255, wherein the non-endogenous ligand comprises a tamoxifen metabolite.

Embodiment 257: The method of any one of Embodiments 252-256, wherein the non-endogenous ligand is endoxifen.

Embodiment 258: The method of Embodiment 252-257, wherein the non-endogenous ligand is administered at a concentration at which the non-endogenous ligand is substantially inactive on a wild-type estrogen receptor alpha of SEQ ID NO: 1.

Embodiment 259: A modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an N413D substitution, an S463P substitution, an L354I substitution, an L384M substitution, and an H524L substitution, with reference to SEQ ID NO: 1.

Embodiment 260: A modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an N413D substitution, an S463P substitution, an M421L substitution, an L354I substitution, and an H524L substitution, with reference to SEQ ID NO: 1.

Embodiment 261: A modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an LA09V substitution, an N413D substitution, an S463P substitution, an M421L substitution, an L384M substitution, and an H524L substitution, with reference to SEQ ID NO: 1.

Embodiment 262: A modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an L409V substitution, an Q414E substitution, an N413D substitution, an S463P substitution, an M517A substitution, and an H524L substitution, with reference to SEQ ID NO: 1.

Embodiment 263: A modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an L409V substitution, an N413D substitution, an S463P substitution, an M517A substitution, an M421L substitution, an L354I substitution, and an H524L substitution, with reference to SEQ ID NO: 1.

Embodiment 264: A modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an Q414E substitution, an N413D substitution, an S463P substitution, an M421L substitution, an L354I substitution, an L384M substitution, and an H524L substitution, with reference to SEQ ID NO: 1.

Embodiment 265: A modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an L409V substitution, an N413D substitution, an S463P substitution, an M517A substitution, an M421L substitution, and an H524L substitution, with reference to SEQ ID NO: 1.

Embodiment 266: A modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an L409V substitution, an Q414E substitution, an N413D substitution, an S463P substitution, an L354I substitution, and an H524L substitution, with reference to SEQ ID NO: 1.

Embodiment 267: A modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an L409V substitution, an N413D substitution, an S463P substitution, an M421L substitution, an L354I substitution, an L384M substitution, and an H524L substitution, with reference to SEQ ID NO: 1.

Embodiment 268: A modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an Q414E substitution, an N413D substitution, an S463P substitution, an M517A substitution, an M421L substitution, an L354I substitution, and an H524L substitution, with reference to SEQ ID NO: 1.

Embodiment 269: A modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an N413D substitution, an S463P substitution, an M517A substitution, an L384M substitution, and an H524L substitution, with reference to SEQ ID NO: 1.

Embodiment 270: A modified estrogen receptor ligand binding domain (ER-LBD) corresponding to a hormone binding domain of a reference human estrogen receptor sequence (SEQ ID NO: 1), wherein the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an L409V substitution, an N413D substitution, an S463P substitution, an M517A substitution, and an H524L substitution, with reference to SEQ ID NO: 1.

Embodiment 271: A modified ER-LBD comprising an amino acid sequence at least 80%, 85%, 90%, 95%, 97.5%, 98%, 99%, or 100% identical to any one of: SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, and SEQ ID NO: 114.

Embodiment 272: The modified ER-LBD of any one of Embodiments 259-271, wherein the modified ER-LBD has greater sensitivity and/or selectivity to a non-endogenous ligand as compared to an ER-LBD comprising the amino acid sequence of SEQ ID NO: 2, or as compared to an endogenous ligand as a result of the one or more additional amino acid substitutions.

Embodiment 273: The modified ER-LBD of Embodiment 272, wherein the non-endogenous ligand is selected from the group consisting of: 4-hydroxytamoxifen, N-desmethyltamoxifen, tamoxifen-N-oxide, tamoxifen, and endoxifen.

Embodiment 274: The modified ER-LBD of any one of Embodiments 272-273, wherein the endogenous ligand is estradiol.

Embodiment 275: The modified ER-LBD of any one of Embodiments 259-274, wherein the modified ER-LBD further comprises a V595A amino acid substitution.

Embodiment 276: A chimeric protein comprising a polypeptide of interest fused to the modified ER-LBD of any one of Embodiments 259-275.

Embodiment 277: The chimeric protein of Embodiment 276, wherein the polypeptide of interest comprises a nucleic acid binding domain.

Embodiment 278: The chimeric protein of Embodiment 277, wherein the nucleic acid binding domain comprises a zinc finger domain.

Embodiment 279: The chimeric protein of Embodiment 278, wherein the zinc finger domain comprises the sequence as set forth in SEQ ID NO: 57 or SEQ ID NO: 84.

Embodiment 280: The chimeric protein of any one of Embodiments 276-279, wherein the chimeric protein comprises a chimeric transcription factor, and wherein the polypeptide of interest comprises a nucleic acid binding domain and a transcriptional modulator domain.

Embodiment 281: The chimeric protein of Embodiment 280, wherein the transcriptional modular domain is a transcriptional activator.

Embodiment 282: The chimeric protein of Embodiment 281, wherein the transcriptional activator is selected from the group consisting of: a Herpes Simplex Virus Protein 16 (VP16) activation domain; an activation domain comprising four tandem copies of VP16; a VP64 activation domain; a p65 activation domain of NFκB (p65); an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase core domain of the human E1A-associated protein p300 (p300 HAT core activation domain). the transcriptional activator is selected from the group consisting of: a Herpes Simplex Virus Protein 16 (VP16) activation domain; an activation domain comprising four tandem copies of VP16; a VP64 activation domain; a p65 activation domain of NFκB (p65); an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase core domain of the human E1A-associated protein p300 (p300 HAT core activation domain).

Embodiment 283: The chimeric protein of Embodiment 282, wherein the transcriptional activator is a p65 transcriptional activator comprising the amino acid sequence of

(SEQ ID NO: 64)
DEFPTMVFPSGQISQASALAPAPPQVLPQAPAPAPAPAMVSALAQAPAPV
PVLAPGPPQAVAPPAPKPTQAGEGTLSEALLQLQFDDEDLGALLGNSTDP
AVFTDLASVDNSEFQQLLNQGIPVAPHTTEPMLMEYPEAITRLVTGAQRP
PDPAPAPLGAPGLPNGLLSGDEDFSSIADMDFSALLSQISS.

Embodiment 284: An isolated polynucleotide molecule comprising a nucleotide sequence encoding the modified ER-LBD of any one of Embodiments 259-275 or the chimeric protein of any one of Embodiments 276-283.

Embodiment 285: A heterologous construct comprising a promoter operatively linked to the polynucleotide molecule of Embodiment 284 or 285.

Embodiment 286: A cell comprising the heterologous construct of Embodiment 285.

Embodiment 287: A molecular switch for modulating transcription of a gene of interest, comprising:
a. The chimeric protein or a heterologous construct encoding the chimeric protein of any one of Embodiments 276-283, wherein the chimeric protein binds to a chimeric transcription factor-responsive (CTF-responsive) promoter operably linked to the gene of interest; and
b. a non-endogenous ligand, wherein binding of the non-endogenous ligand to the modified ER-LBD induces the chimeric protein to modulate transcription of the gene of interest.

Embodiment 288: The molecular switch of Embodiment 287, wherein the non-endogenous ligand is selected from: 4-hydroxytamoxifen, N-desmethyltamoxifen, tamoxifen-N-oxide, tamoxifen, and endoxifen.

Embodiment 289: The molecular switch of any one of Embodiments 287-288, wherein the gene of interest encodes a polypeptide selected from the group consisting of: a cytokine, a chemokine, a homing molecule, a growth factor, a cell death regulator, a co-activation molecule, a tumor microenvironment modifier a, a receptor, a ligand, an antibody, a polynucleotide, a peptide, and an enzyme.

Embodiment 290: The molecular switch of any one of Embodiments 287-289, further comprising an additional construct comprising the CTF-responsive promoter operably linked to the gene of interest.

Embodiment 291: The molecular switch of Embodiment 290, wherein the heterologous construct and the additional construct are comprised in a single vector.

Embodiment 292: The molecular switch of Embodiment 290, wherein the heterologous construct is comprised in a first vector and the additional construct is comprised in a second vector.

Embodiment 293: A method of modulating transcription of a gene of interest, comprising:
a. transforming a cell with (i) a heterologous construct encoding the chimeric protein of any one of Embodiments 276-283 and (ii) an additional construct comprising a chimeric transcription factor-responsive (CTF-responsive) promoter operably linked to the gene of interest; and
b. inducing the chimeric protein to modulate transcription of the gene of interest by contacting the transformed cell with a non-endogenous ligand.

Embodiment 294: A method of modulating transcription of a gene of interest, comprising contacting a transformed cell with a non-endogenous ligand, wherein the transformed cell comprises (i) a heterologous construct encoding the chimeric protein of any one of Embodiments 276-283 and (ii) an additional construct comprising a chimeric transcription factor-responsive (CTF-responsive) promoter operably linked to the gene of interest.

Embodiment 295: The method of Embodiment 294 or 295, wherein the modulating transcription comprises activating transcription of the gene of interest.

Embodiment 296: A method of modulating localization of a chimeric protein, comprising:
i. transforming a cell with a heterologous construct encoding the chimeric protein of any one of Embodiments 276-283: and
ii. inducing nuclear localization of the chimeric protein by contacting the transformed cell with a non-endogenous ligand.

Embodiment 297: A method of modulating localization of a chimeric protein, comprising contacting a transformed cell with a non-endogenous ligand, wherein the transformed cell comprises a heterologous construct encoding the chimeric protein of any one of Embodiments 276-283, wherein the contacting induces nuclear localization of the chimeric protein.

Embodiment 298: The method of any one of Embodiments 293-297, wherein:
i. the method further comprises culturing the transformed cell under conditions suitable for expression of the chimeric protein prior to contacting the transformed cell with the non-endogenous ligand, and/or
ii. the heterologous construct and the additional construct are comprised in a single vector or the heterologous construct is comprised in a first vector and the additional construct is comprised in a second vector, and/or
iii. the non-endogenous ligand is selected from the group consisting of: 4-hydroxytamoxifen, N-desmethyltamoxifen, tamoxifen-N-oxide, tamoxifen, and endoxifen, and/or
   the non-endogenous ligand is administered at a concentration at which the non-endogenous ligand is substantially inactive on a wild-type estrogen receptor alpha of SEQ ID NO: 1.

### EXAMPLES

Below are examples of specific embodiments for carrying out the present disclosure. The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1

### ERT2 Mutations Predicted to Modulate Ligand Binding

In silico modeling was conducted for 4-OHT, endoxifen and estradiol binding to a mutant form of estrogen receptor alpha known as ERT2, to identify mutations predicted to have increased sensitivity to 4-OHT, as compared to an ERT2 of SEQ ID NO: 2.

### Materials and Methods

Available crystal structures of a complex between Estradiol and ERα (PDB: 1QKU, resolution 3.2 Å) and between 4-OHT and ERα (PDB: 3ERT, resolution 1.9 Å) were used to generate models of the complexes between Estradiol and ERT2, 4-OHT and ERT2, and Endoxifen and ERT2. The ERT2 sequence differs from ERα by three residues (G400V/M543A/L544A). Only residues from 306 to 551 were used in the structural models as the available structures were all resolved with only this region.

Using standard protocols (Kannan et al. ACS Omega. 2017 Nov. 30; 2 (11): 7881-7891.), MD simulations were carried out for apo ERT2, ERT2—Estradiol, ERT2—4-OHT and ERT2—Endoxifen complexes (each simulation was carried out for 100 ns in triplicate each). Both the ERT2 and the bound ligand/drug remained stable during the simulations (using standard measures). The conformations generated during the last half (50 ns) of the simulations (the simulations are deemed to have equilibrated) were used for subsequent analyses.

### Results

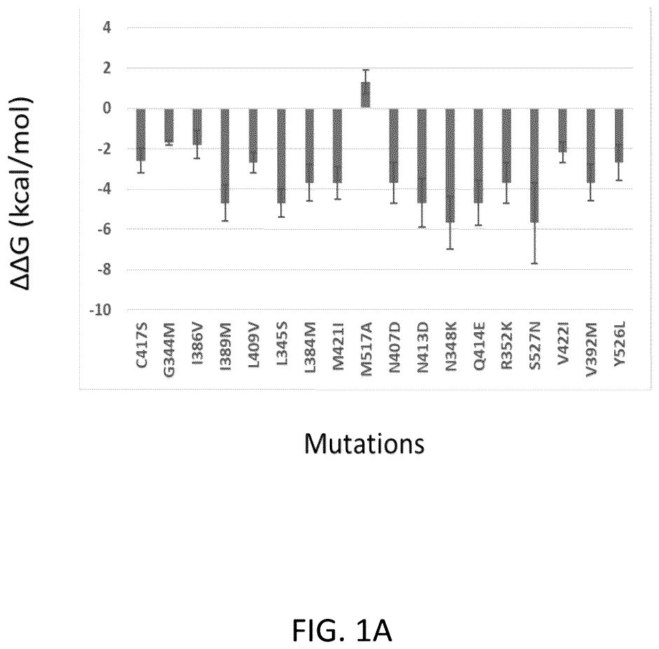

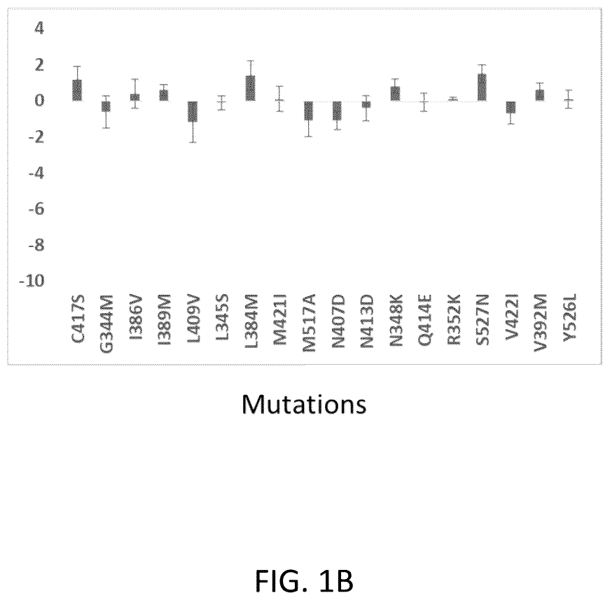

A first set of mutations was analyzed in silico for improved 4-OHT binding. Eighteen mutations to residues in the ligand binding pocket were selected based on amino acids present at the homologous position for other estrogen receptor proteins. Of the 18 selected mutations, 17 of the mutants bind tighter than wild type ERT2 by at least 1.8 kcal/mol; only the M517A mutation appears to destabilize the binding of 4-OHT (FIG. 1A). Next, binding energy calculations were carried out to see the effect of the mutation on the binding of estradiol. Compared to 4-OHT (in which all mutations except M517A favor the binding as indicated by negative ΔΔG values) most mutations (FIG. 1B) had negligible effect on the binding of estradiol (ΔΔG values for all the mutations are within 2 kcal/mol, as compared to the ΔΔG values of 4-OHT which is >2 kcal/mol for most of the mutations). Only mutations L409V, M517A and N407D exhibited increased binding to estradiol of greater than 1 kcal/mol, but both L409V and N407D bind tighter to 4-OHT by 3 and 4 kcal/mol respectively. The first set of mutations is shown below in Table 6.

TABLE 6
Mutations

| | | |
|---|---|---|
| G344M | I389M | C417S |
| L345S | V392M | M421I |
| N348K | N407D | V422I |
| R352K | L409V | M517A |
| L384M | N413D | Y526L |
| I386V | Q414E | S527N |

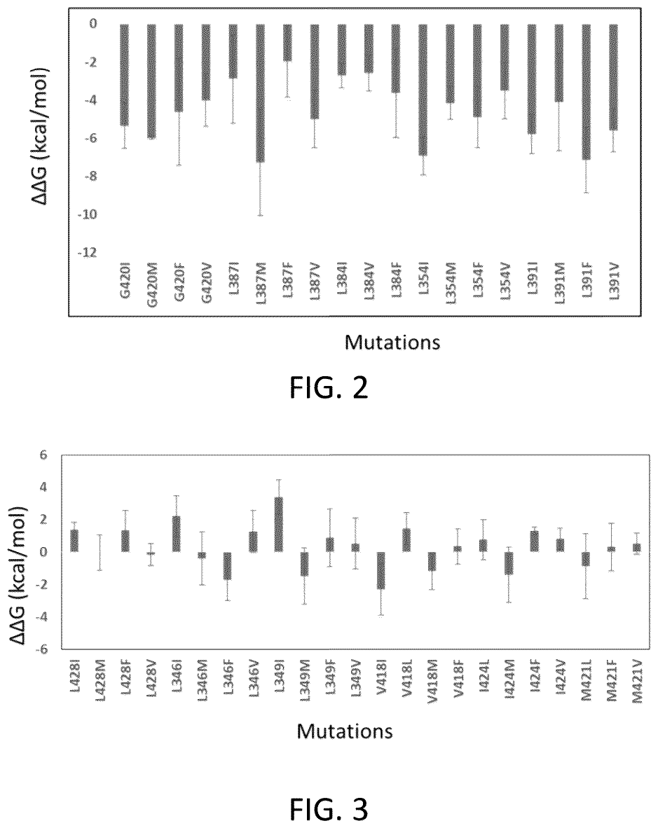

A second set of mutations was analyzed in silico for improved 4-OHT binding. Molecular docking simulations were conducted for 4-OHT and estradiol binding to ERT2, for nineteen different mutations at five additional sites at the ligand binding pocket (in addition to those shown in Table 6), to identify further mutants with increased sensitivity to 4-OHT, as compared to wild-type ERT2. Binding energy calculations were carried out consistent with the calculations performed for the first set of mutations. All nineteen of the mutations exhibited improved binding to 4-OHT in the range of 1.8 kcal/mol to 7 kcal/mol (see FIG. 2). The second set of mutations is shown in Table 7.

TABLE 7
Mutations

| | | |
|---|---|---|
| L354I | L387I | L391V |
| L354M | L387M | G420I |
| L354F | L387F | G420M |
| L354V | L387V | G420F |
| L384I | L391I | G420V |
| L384V | L391M | |
| L384F | L391F | |

A third set of mutations was analyzed in silico for improved 4-OHT binding. A total of 23 mutations at an additional six residue positions in the ligand binding pocket (residues 428, 346, 349, 418, 421, and 424) were chosen for molecular docking simulations. Binding energy calculations were carried out consistent with the calculations performed for the first set of mutations. Six of the mutations (L346F, L349M, V418I, V418M, 1424M, and M421L) exhibited improved binding to 4-OHT by at least about 1.5 kcal/mol (FIG. 3). These 23 mutations are shown in Table 8.

TABLE 8
Mutations

| | | |
|---|---|---|
| L346I | V418I | I424M |
| L346M | V418L | I424F |
| L346F | V418M | I424V |
| L346V | V418F | L428I |
| L349I | M421L | L428M |
| L349M | M421F | L428F |
| L349F | M421V | L428V |
| L349V | I424L | |

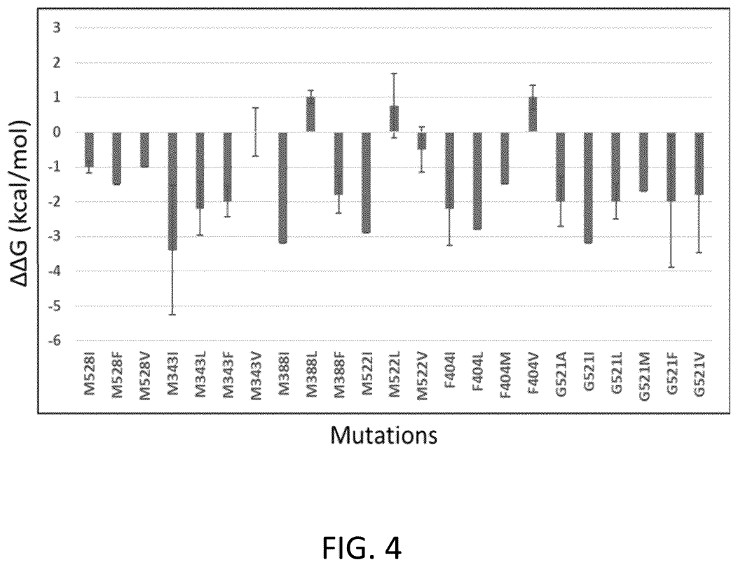

A fourth set of mutations was analyzed in silico for improved 4-OHT binding. A total of 23 mutations at an additional six residue positions in the ligand binding pocket (residues 528, 343, 388, 522, 414, and 521) were chosen for molecular docking simulations. Binding energy calculations were carried out consistent with the calculations performed for the first set of mutations. 18 of the 23 mutations exhibited improved binding to 4-OHT by at least about 1.0 kcal/mol (FIG. 4). The fourth set of mutations is shown in Table 9.

TABLE 9
Mutations

| | | |
|---|---|---|
| M343I | F404L | G521V |
| M343L | F404M | M522I |
| M343F | F404V | M522L |
| M343V | G521A | M522V |
| M388I | G521I | M528I |
| M388L | G521L | M528F |
| M388F | G521M | M528V |
| F404I | G521F | |

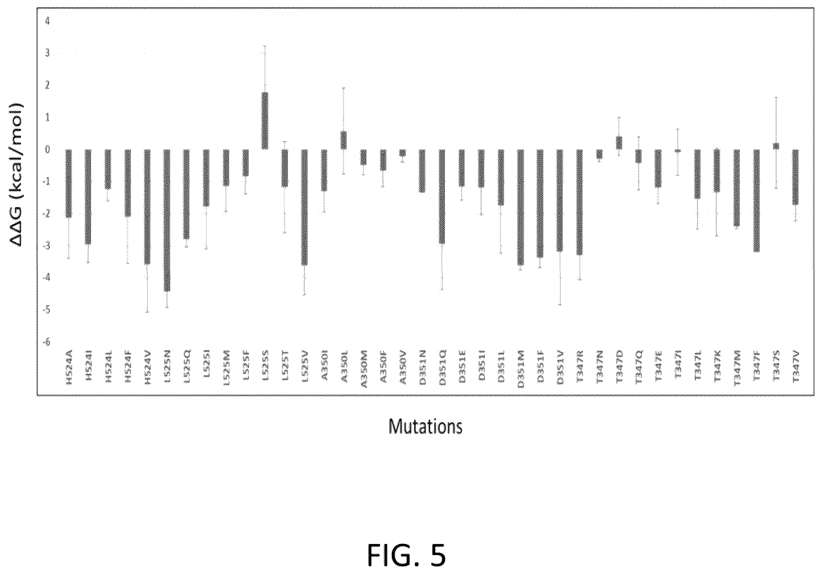

A fifth set of mutations was analyzed in silico for improved 4-OHT binding. A total of 38 mutations at five additional residue positions (residues 524, 525, 347, 350, and 351) were chosen for molecular docking simulations. Binding energy calculations were carried out consistent with the calculations performed for the first set of mutations. 28 of the 38 mutations exhibited improved binding to 4-OHT by at least about 1.0 kcal/mol and up to about 4.5 kcal/mol (FIG. 5). The fifth set of mutations is shown in Table 10.

TABLE 10
Mutations

| | |
|---|---|
| H524A | T347S |
| H524I | A350I |
| H524L | A350L |
| H524F | A350M |
| H524V | A350F |
| L525N | A350V |
| L525Q | D351N |
| L525I | D351Q |
| L525M | D351E |
| L525F | D351I |
| L525S | D351L |
| L525T | D351M |
| L525V | D351F |
| T347V | D351V |

All of the mutations for sets 1-5 were further analyzed with molecular docking simulations for binding to endoxifen and estradiol to determine the energy of binding to endoxifen and estradiol (calculated as ΔΔG in kcal/mol). Additionally, the difference between the binding energy of endoxifen binding as compared to estradiol binding was calculated as ΔΔΔG values. A summary of the binding energies for each of 4-OHT, endoxifen, and estradiol, and of the binding energy differences of 4-OHT and endoxifen as compared to estradiol binding is shown in Table 11.

TABLE 11

| | 4-OHT | | EST | 4-OHT-EST | END-EST |
|---|---|---|---|---|---|
| | (ΔΔG) | END(ΔΔG) | (ΔΔG) | (ΔΔΔG) | (ΔΔΔG) |
| | (kcal/mol) | (kcal/mol) | (kcal/mol) | (kcal/mol) | (kcal/mol) |
| M343I | −3.4 | −2.2 | 1.5 | −4.9 | −3.7 |
| M343L | −2.2 | −1.6 | 0.9 | −3.1 | −2.6 |
| M343V | 0 | −0.7 | 1.8 | −1.8 | −2.5 |
| M343F | −2 | −1.6 | 2.2 | −4.2 | −3.8 |
| G344M | −1.6 | −1.4 | −0.6 | −1 | −0.8 |
| L345S | −4.3 | 1.1 | −0.1 | −4.2 | 1.2 |
| L346I | 2.2 | −0.8 | −0.9 | 3.1 | 0.1 |
| L346M | −0.4 | −0.8 | −0.5 | 0.1 | −0.3 |
| L346V | 1.3 | 0.8 | 0.5 | 0.8 | 0.3 |
| L346F | −1.7 | −0.8 | −0.1 | −1.6 | −0.6 |
| T347I | −0.1 | −0.7 | 0.4 | −0.5 | −1.1 |
| T347L | −1.6 | −1.2 | 0.5 | −2 | −1.7 |
| T347M | −2.4 | −2.3 | −1.9 | −0.5 | −0.3 |
| T347N | −0.3 | −2.7 | 1.1 | −1.4 | −3.8 |
| T347R | −3.3 | −4.3 | −0.8 | −2.5 | −3.5 |
| T347V | −1.7 | −0.9 | 0 | −1.7 | −0.9 |
| T347D | 0.4 | 3.8 | 0.4 | 0 | 3.4 |
| T347E | −1.2 | 2.5 | 1.1 | −2.3 | 1.4 |
| T347F | −3.2 | −2.2 | 0.1 | −3.3 | −2.2 |
| T347K | −1.3 | −2.5 | 1.4 | −2.7 | −3.9 |
| T347Q | −0.4 | 0.4 | 1.3 | −1.8 | −0.9 |
| T347S | 0.2 | −2.7 | 1.1 | −0.9 | −3.7 |
| N348K | −5.5 | −3.6 | 0.8 | −6.3 | −4.4 |
| L349I | 3.4 | 1.4 | 2 | 1.4 | −0.6 |
| L349M | −1.5 | −2 | 2.6 | −4.1 | −4.6 |
| L349V | 0.5 | 1.5 | 1.7 | −1.2 | −0.2 |
| L349F | 0.9 | −0.8 | −0.7 | 1.6 | −0.1 |
| A350L | 0.6 | −0.6 | 1 | −0.4 | −1.6 |
| A350M | −0.5 | −2 | 1.3 | −1.8 | −3.3 |
| A350V | −0.2 | −1.1 | 0.5 | −0.7 | −1.5 |
| A350F | −0.7 | −2.3 | −1 | 0.4 | −1.3 |
| A350I | −1.3 | −3.8 | 0.6 | −1.9 | −4.4 |
| D351I | −1.2 | −3.5 | 2 | −3.2 | −5.5 |
| D351L | −1.7 | −3.2 | 0.4 | −2.1 | −3.6 |
| D351M | −3.6 | −3 | −0.2 | −3.4 | −2.9 |
| D351N | −1.3 | −2.1 | 1.8 | −3.2 | −4 |
| D351V | −3.2 | −3.8 | 0.6 | −3.8 | −4.4 |
| D351E | −1.2 | 1 | 0.7 | −1.8 | 0.3 |
| D351F | −3.4 | −2.2 | 1.2 | −4.6 | −3.4 |
| D351Q | −3 | 0.1 | 0.3 | −3.2 | −0.2 |
| R352K | −3.8 | −2.5 | 0.1 | −3.9 | −2.6 |
| L354I | −6.9 | −2.5 | 0.9 | −7.8 | −3.4 |
| L354M | −4.2 | −1.3 | 0.9 | −5.1 | −2.2 |
| L354V | −3.5 | −2.5 | 1.5 | −5 | −4 |
| L354F | −4.9 | −2.6 | 0.7 | −5.6 | −3.3 |
| L384I | −2.7 | −4.6 | 0.3 | −3 | −4.9 |
| L384M | −3.3 | 0 | 1.4 | −4.7 | −1.4 |
| L384V | −3.6 | −2.5 | 3.2 | −6.9 | −5.7 |
| L384F | −2.6 | −0.4 | 3 | −5.5 | −3.3 |
| I386V | −1.8 | −3.4 | 0.4 | −2.2 | −3.8 |
| L387I | −2.9 | −2.6 | 0.4 | −3.2 | −3 |
| L387M | −7.3 | −1.9 | 0.3 | −7.5 | −2.2 |
| L387V | −5 | −2.4 | −0.7 | −4.3 | −1.6 |
| L387F | −2 | −0.4 | 1.1 | −3 | −1.4 |
| M388I | −3.2 | −1.7 | 0.3 | −3.5 | −2 |
| M388L | 1 | −0.7 | 1.9 | −0.9 | −2.6 |
| M388F | −1.8 | −1.6 | 1.2 | −3 | −2.8 |
| I389M | −4.6 | −2.1 | 0.6 | −5.2 | −2.7 |
| L391I | −5.8 | −2.8 | 2 | −7.8 | −4.8 |
| L391M | −4.1 | −4.5 | 0 | −4.1 | −4.5 |
| L391V | −5.6 | −3.4 | 1.5 | −7.1 | −4.9 |
| L391F | −7.2 | −1.5 | 0.1 | −7.3 | −1.6 |
| V392M | −3.9 | −2.6 | 0.6 | −4.5 | −3.2 |
| F404I | −2.2 | −1.3 | 2 | −4.2 | −3.4 |
| F404L | −2.8 | −1.7 | −0.5 | −2.3 | −1.2 |
| F404M | −1.5 | −0.3 | 2.2 | −3.7 | −2.4 |
| F404V | 1 | −1 | 1.4 | −0.4 | −2.4 |
| N407D | −3.5 | −2.2 | −1.1 | −2.4 | −1.1 |
| L409V | −2.5 | −1.8 | −1.2 | −1.3 | −0.6 |
| N413D | −4.7 | −0.8 | −0.4 | −4.3 | −0.4 |
| Q414E | −4.5 | −1.8 | −0.1 | −4.4 | −1.7 |
| C417S | −2.6 | −1 | 1.2 | −3.8 | −2.2 |
| V418I | −2.3 | −1.1 | 1.9 | −4.2 | −3.1 |
| V418L | 1.5 | −1.3 | 2 | −0.5 | −3.2 |
| V418M | −1.2 | −1.8 | 1.4 | −2.6 | −3.2 |
| V418F | 0.4 | −2.6 | 0.6 | −0.3 | −3.2 |
| G420I | −5.3 | −2.8 | −0.2 | −5.1 | −2.6 |
| G420M | −6 | −3.9 | −1.2 | −4.8 | −2.7 |
| G420V | −4 | −2.5 | 0.1 | −4.1 | −2.6 |
| G420F | −4.6 | −4 | 3.3 | −7.9 | −7.3 |
| M421I | −3.3 | −2.1 | 0.1 | −3.4 | −2.2 |
| M421L | −0.9 | −1.7 | 0 | −0.9 | −1.7 |
| M421V | 0.5 | 0.4 | 0.4 | 0.1 | 0 |
| M421F | 0.3 | −1.4 | −0.7 | 1 | −0.8 |
| V422I | −2.4 | −2.2 | −0.7 | −1.7 | −1.5 |
| I424L | 0.8 | −0.8 | −0.6 | 1.3 | −0.2 |
| I424M | −1.4 | −1.8 | 0.3 | −1.7 | −2 |
| I424V | 0.8 | −1.8 | 3.1 | −2.3 | −4.9 |
| I424F | 1.3 | −1.4 | 0.8 | 0.5 | −2.2 |
| L428I | 1.3 | −0.5 | 0.5 | 0.8 | −1.1 |
| L428M | 0 | −1.9 | 0.9 | −0.9 | −2.8 |
| L428V | −0.2 | −0.1 | 2.3 | −2.4 | −2.4 |
| L428F | 1.3 | 0.7 | 1.3 | 0 | −0.6 |
| M517A | 1.2 | 0.8 | −1.1 | 2.3 | 1.9 |
| G521A | −2 | 0 | 0.2 | −2.2 | −0.2 |
| G521I | −3.2 | −0.3 | 2.3 | −5.5 | −2.6 |
| G521L | −2 | −1 | 1.9 | −3.9 | −2.9 |
| G521M | −1.7 | −2.5 | 3.3 | −5 | −5.8 |
| G521V | −1.8 | −1.1 | 0.5 | −2.3 | −1.6 |
| G521F | −2 | −1.8 | 3.4 | −5.4 | −5.3 |
| M522I | −2.9 | −2 | 1.7 | −4.6 | −3.7 |
| M522L | 0.8 | −0.7 | 0.1 | 0.7 | −0.8 |
| M522V | −0.5 | −1 | −0.7 | 0.2 | −0.3 |
| H524A | −2.1 | −3 | −0.4 | −1.7 | −2.6 |
| H524I | −3 | −3 | 2.8 | −5.8 | −5.8 |
| H524L | −1.2 | −1.3 | 2 | −3.3 | −3.3 |
| H524V | −3.6 | −3.3 | 0.6 | −4.2 | −4 |
| H524F | −2.1 | −3.9 | 2.7 | −4.8 | −6.6 |
| L525I | −1.8 | −2.1 | 0.5 | −2.3 | −2.6 |
| L525M | −1.2 | −1.8 | −0.2 | −0.9 | −1.5 |
| L525N | −4.4 | −1.4 | −0.4 | −4 | −0.9 |
| L525T | −1.2 | −2.3 | 0.6 | −1.7 | −2.9 |
| L525V | −3.6 | −4.5 | −1 | −2.7 | −3.5 |
| L525F | −0.8 | −1.1 | −0.4 | −0.4 | −0.6 |
| L525Q | −2.8 | −3.4 | −1.4 | −1.4 | −2 |
| L525S | 1.8 | −2.4 | 0.3 | 1.5 | −2.7 |
| Y526L | −2.8 | −2 | 0.1 | −2.9 | −2.1 |
| S527N | −5.8 | −3 | 1.5 | −7.3 | −4.5 |
| M528I | −1 | −2.5 | 0.5 | −1.5 | −2.9 |
| M528V | −1 | −1.1 | 3 | −4 | −4.1 |
| M528F | −1.5 | −1 | 2.5 | −4 | −3.5 |

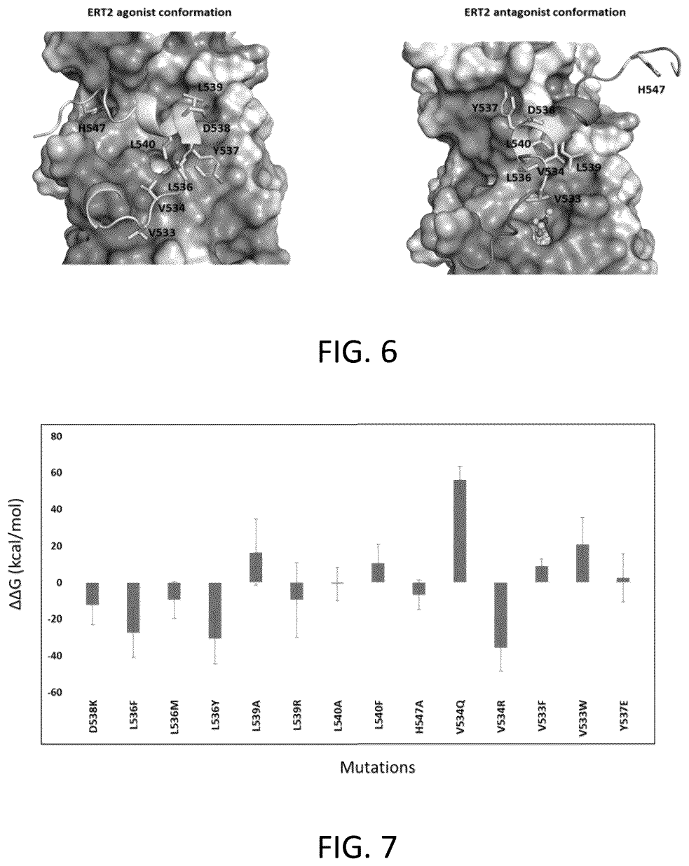

A sixth set of mutations was analyzed in silico to identify mutants that destabilize the agonist-bound confirmation (i.e., the estradiol-bound conformation) and/or stabilize the antagonist-bound confirmation (i.e., the 4-OHT or endoxifen-bound conformation). A major structural difference between the agonist-bound and antagonist-bound conformations lies in the orientation and docking site of helix 12 (H12, see FIG. 6). A total of 14 mutations at eight residue positions in helix 12 (residues 538, 536, 539, 540, 547, 534, 533, and 537) were chosen for analysis. The difference in free energy of the mutant ERT2 and the wild-type ERT2 in the antagonist-bound conformation, and the difference in free energy of the mutant ERT2 and wild-type ERT2 in the agonist-bound conformation were calculated as AG values. Next, AAG values were calculated, with a negative value indicating that the antagonist-bound conformation of the ERT2 mutant is favored over the agonist-bound conformation of the mutant, and a positive value indicating that the agonist conformation is favored over the antagonist-bound conformation. Seven of the fourteen mutations (D538K, L536F, L536Y, L536M, L539R, H547A, and V534R) stabilized the antagonist confirmation (see FIG. 7). The sixth set of mutations is shown in Table 12.

TABLE 12
Mutations

| | |
|---|---|
| V533F | Y537E |
| V533W | D538K |
| V534R | L539R |
| V534Q | L539A |
| L536F | L540A |
| L536M | L540F |
| L536Y | H547A |

### Example 2

### ERT2 Mutants with Increased Drug Sensitivity Identified by Transfection Screen

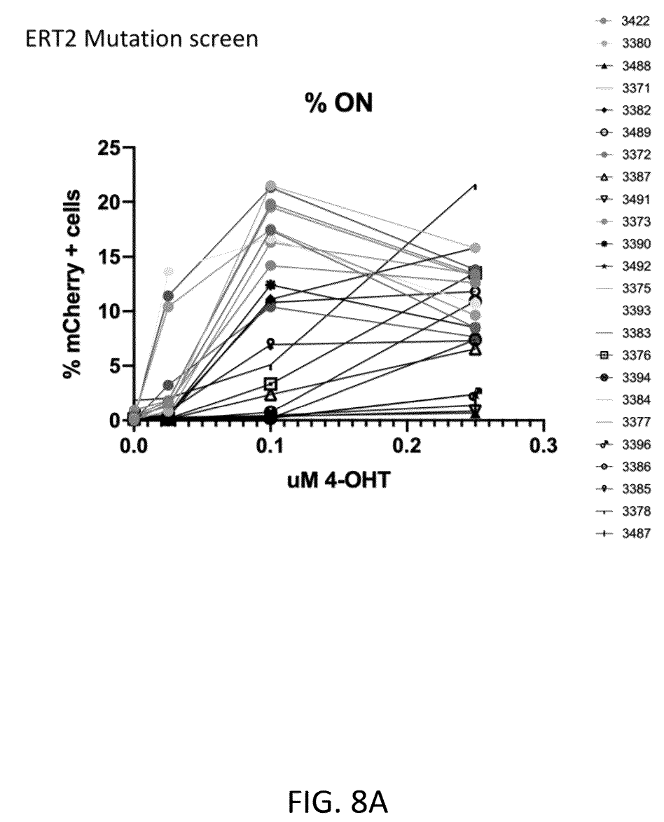

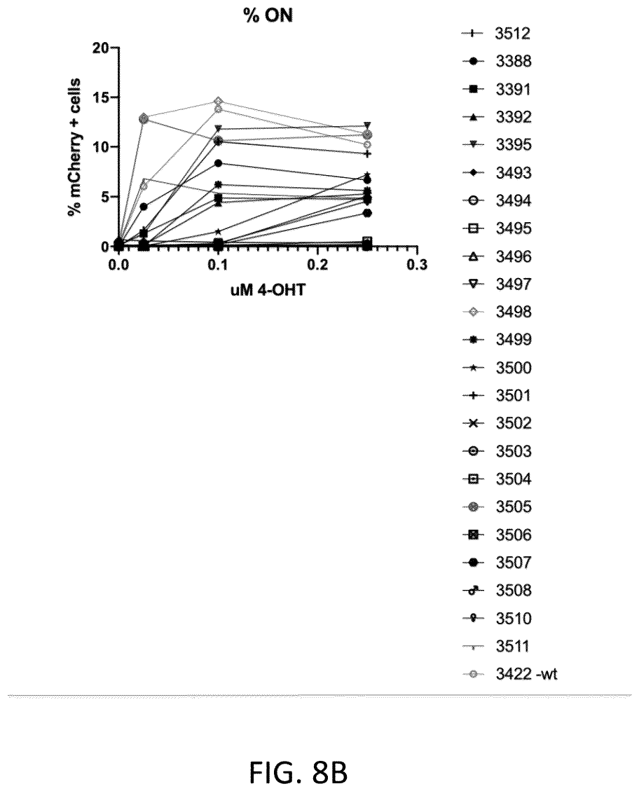

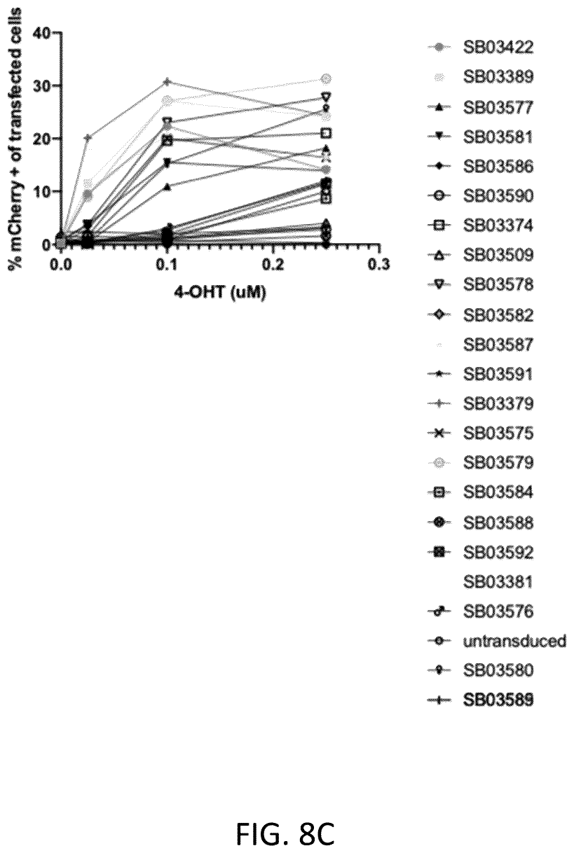

ERT2 mutants identified from the in silico analysis of Example 1 were analyzed by transfection assays for responsiveness to 4-OHT. In a first transfection screen using reporter expression as a read out, constructs encoding ERT2 having mutations described in Example 1 were produced in the background of a “wild-type” ERT2 as shown in SEQ ID NO: 3 (including the G400V/M543A/L544A/V595A quadruple amino acid substitution). Each ERT2 construct included a ZF10-1 domain for DNA binding, a p65 transcriptional activation domain, and the ERT2 mutant including a modified ER-LBD. Each construct was tested for sensitivity to 4-OHT. Each mutant was cloned into an expression construct for transfection in a HEK293T+YBTATA_mCherry reporter cell line. In a second transfection screen, constructs encoding additional ERT2 mutants described in Example 1 were produced and tested for sensitivity to 4-OHT. For the screens, the cells were treated with three different concentrations of 4-OHT (0.025, 0.1, and 0.25 uM) and then assayed for mCherry expression by fluorescence-activated cell sorting (FACS) (FIGS. 8A-8C, FIGS. 9A-9C, and FIGS. 10A-10C).

### Materials and Methods

HEK293T cells were transduced with a lentivirus encoding a synthetic promoter comprised of 4 ZF10-1 binding sites linked to a YBTATA minimal promoter. This synthetic promoter drives expression of mCherry. Cells from this cell line were called “reporter cells.”

On day 1, reporter cells were plated at 1.5e5 cells/well in a 24 well plate. On day 2, cells were transfected with ERT mutants. A mix of 0.6 ug DNA, 1.8 uL Fugene, and 30 uL Optimem was made for each well where the DNA encodes ZF10-1 fused to p65 and the ERT2 mutant including a modified ER-LBD. In some screens a plasmid encoding GFP was included as a control to select transfected cells by flow cytometry. On day 3, cells were split at a ratio of 1:20 and seeded in a 96 well plate. Cells were treated with 0, 0.025, 0.1, or 0.25 uM 4-OHT. On day 5, media was removed and cells were trypsinized and then resuspended in FACS buffer plus Sytox Red (fluoresces in APC channel) viability dye. Cells were run on a flow cytometer and gated by FSC/SSC for cells, FSC/Sytox Red—for live cells, FSC/FSC-Width for single cells, and where possible GFP+ for transfected cells (if transfection control was included). The percent of mCherry positive cells at each drug concentration was plotted and compared to wildtype ERT2, and mutants that were more sensitive to 4-OHT were identified.

### Results

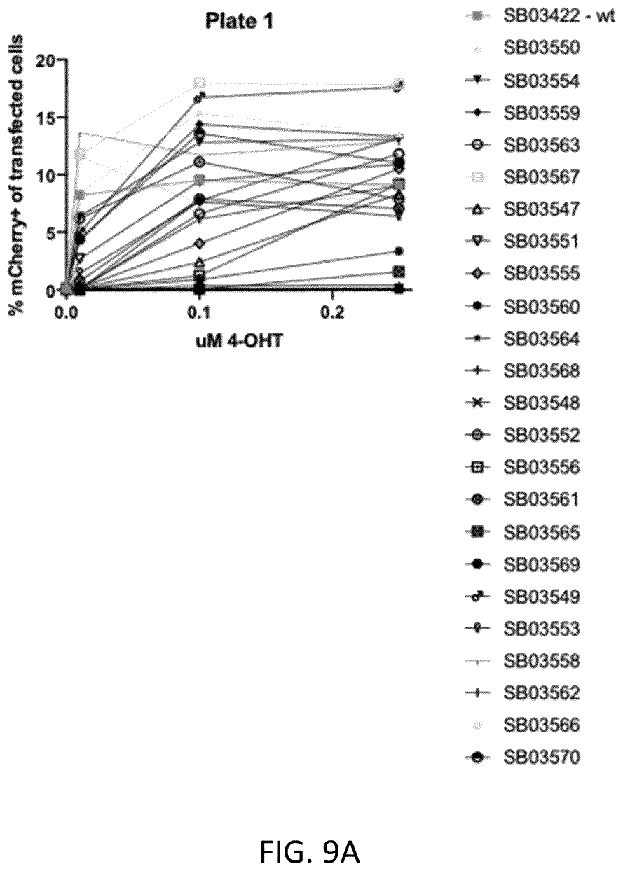

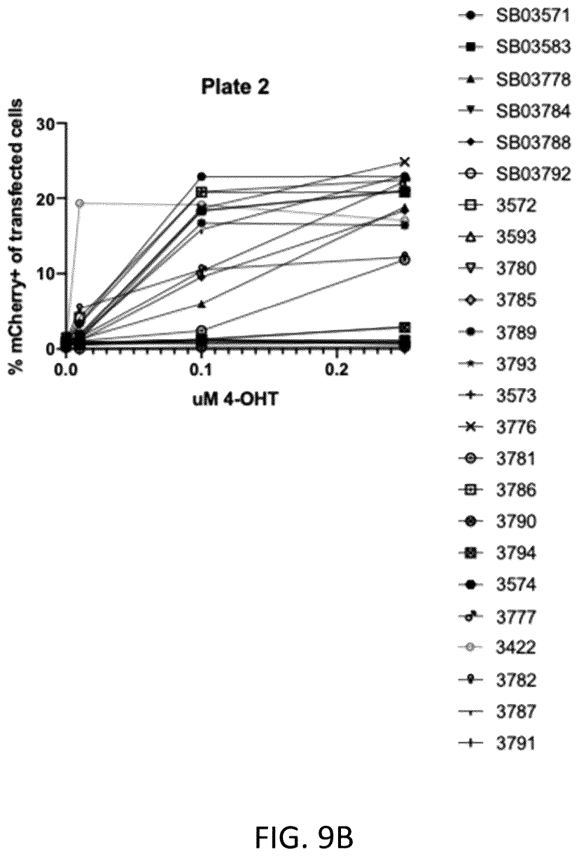

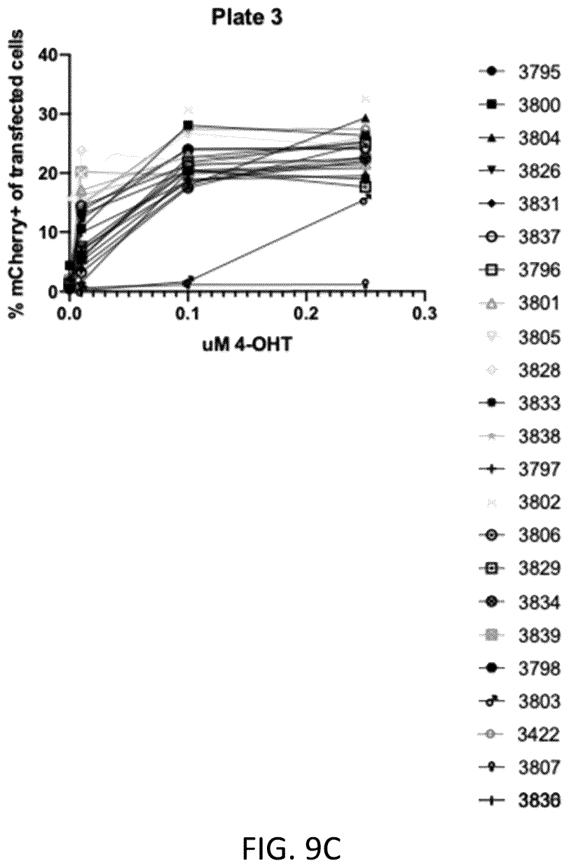

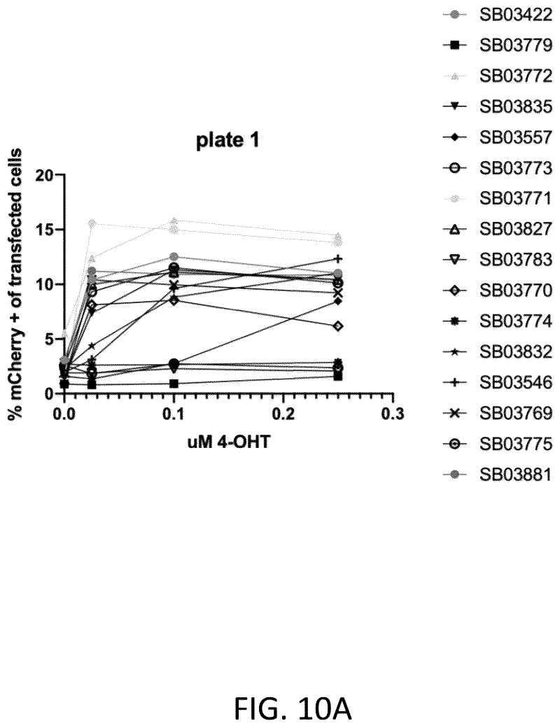

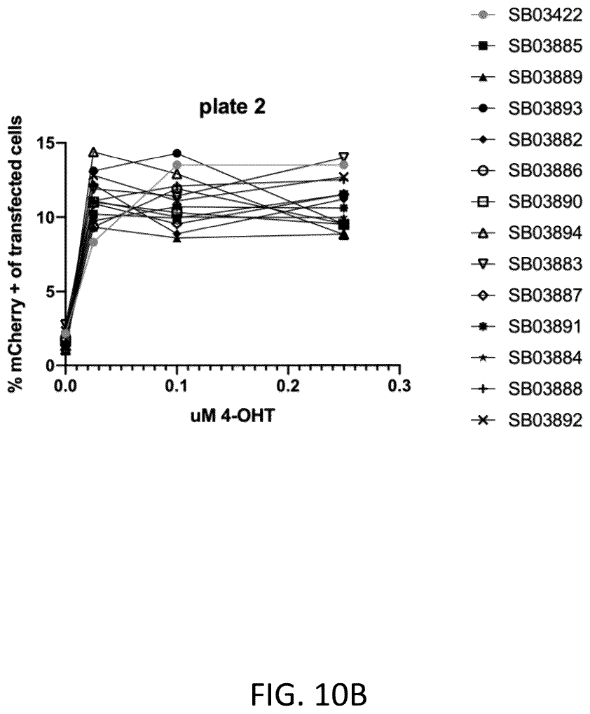

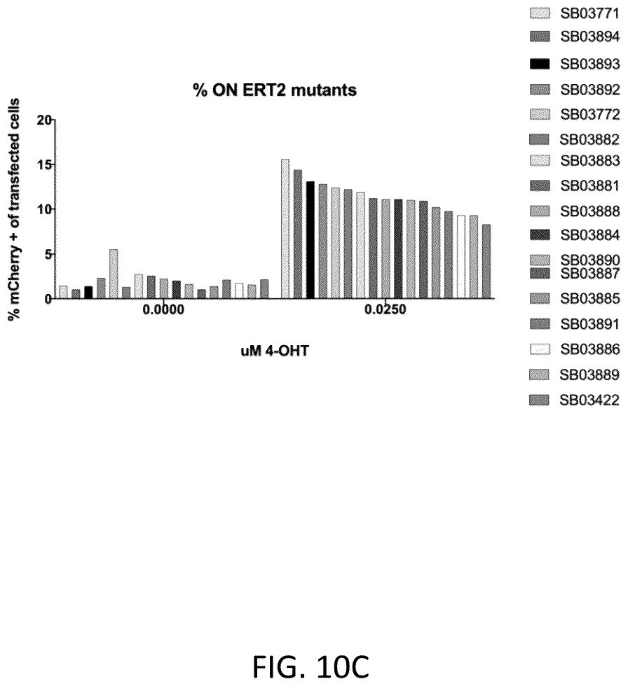

As shown in FIGS. 8A-8C, FIGS. 9A-9C, and FIGS. 10A-10C, the transfection screens identified mutants with improved induction of mCherry expression as compared to an ERT2 (SEQ ID NO: 3). In the first transfection screen, improved expression induction was observed for seventeen of the modified ER-LBD containing constructs: L354I (SB03498), L391V (SB03505), Q414E (SB03383), L409V (SB03375), S463P (SB03393), L384M (SB03377), L354I+L384M (SB03511), N413D (SB03381), M517A (SB03379), G344M (SB03372), I386V (SB03373), N407D (SB03380), C417S (SB03371), R352K (SB03384), Y537S (SB03389), M388F (SB03579), and G521A (SB03587), with the greatest improvement of expression induced by the following seven mutants: L354I (SB03498), L391V (SB03505), Q414E (SB03383), L409V (SB03375), and S463P (SB03393), (FIGS. 8A-8C). In the second transfection screen, improved expression induction was observed for ten of the modified ER-LBD containing constructs: 1424L (SB03558), M421L (SB03566), M421F (SB03567), T347E (SB03801), L536M (SB03828), Y537E (SB03839), T347I (SB03802), and T347M (SB03805), V418I (SB03550), and V533W (SB03838), with strong improvement of expression induced by the following six modified ER-LBD containing constructs: 1424L (SB03558), M421L (SB03566), M421F (SB03567), T347E (SB03801), L536M (SB03828), and Y537E (SB03839) (FIGS. 9A-9C). In the third transfection screen (FIGS. 10A-10C), improved expression induction was observed for the modified ER-LBD containing constructs shown in Table 13.

TABLE 13

| Construct | Description |
|---|---|
| SB03771 | H524L |
| SB03894 | L354I + Q414E |
| SB03893 | L354I + L384M + |
| | Q414E |
| SB03892 | L391V + Q414E |
| SB03772 | H524F |
| SB03882 | L409V + L391V |
| SB03883 | L409V + |
| | L354I + L384M |
| SB03881 | L409V + S463P |
| SB03888 | S463P + L354I |
| SB03884 | L409V + L354I |
| SB03890 | L391V + |
| | L354I + L384M |
| SB03887 | S463P + |
| | L354I + L384M |
| SB03885 | L409V + Q414E |
| SB03891 | L391V + L354I |
| SB03886 | S463P + L391V |
| SB03889 | S463P + Q414E |

### Example 3

### Mutants with Increased Drug Sensitivity Identified by Transduction Screen

ERT2 mutants were analyzed by three transduction screens for the ability to induce reporter expression in response to 4-OHT. The mutants L354I+L384M (identified in the first transfection screen) was included in all three transduction screens. Lentiviral vectors were cloned encoding the ERT2 mutants that demonstrated improved response to 4-OHT in the transfection screen from Example 2. The reporter cell line as described in Example 2 was transduced with lentiviruses encoding the ERT2 mutants, and the ability of the mutants to induce mCherry expression in response to a variety of 4-OHT concentrations was assessed.

### Materials and Methods

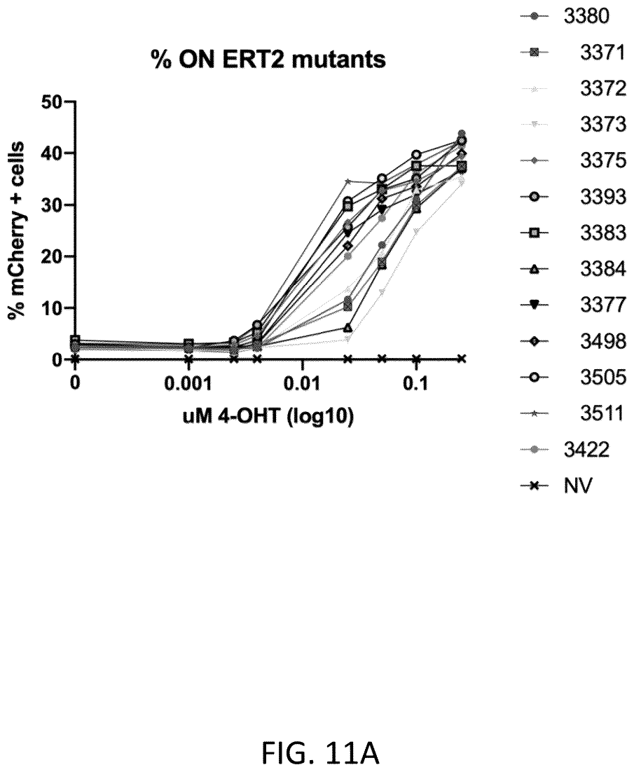

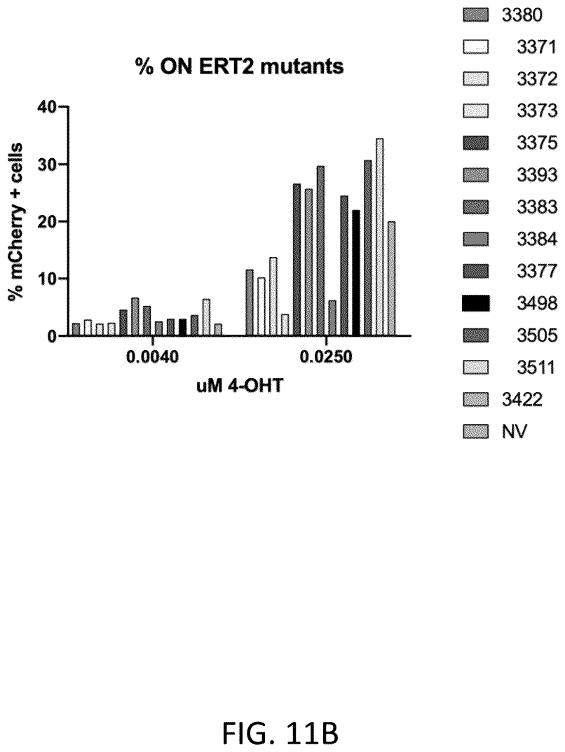

For the transduction screens, on day 1, reporter cells were plated at 2e5 cells/well in a 12 well plate. On day 2, cells were transduced with lentivirus encoding lead ERT mutants from the transfection screen. On days 3 and 4, cells were passaged to maintain <90% confluency on the plate. On day 5, cells were seeded into 96 well plates and treated with 0, 0.001, 0.0025, 0.004, 0.025, 0.05, 0.1, or 0.25 uM 4-OHT. On day 8, media was removed and cells were trypsinized and then resuspended in FACS buffer plus Sytox Red (fluoresces in APC channel) viability dye. Cells were run on a flow cytometer and gated by FSC/SSC for cells, FSC/Sytox Red—for live cells, the percent of mCherry positive cells at each drug concentration was plotted and compared to wildtype ERT2 to find more sensitive mutants (FIG. 11A). The percent of mCherry positive cells at two of the drug concentrations, 0.004 and 0.025 uM 4-OHT, are also shown in a bar graph (FIG. 11B).

### Results

As shown in FIGS. 11A and 11B, the first transduction screen confirmed improved 4-OHT response for several mutants identified as having improved 4-OHT binding in a transfection screen from Example 2. In particular, the mutants L354I, L391V, Q414E, L409V, S463P, L384M, and L354I+L384M all demonstrated an improved 4-OHT response as compared to a wild-type ERT2 (construct 3422, SEQ ID NO: 3). ERT2 mutants demonstrating improved 4-OHT binding in the first transduction screen are shown in Table 14.

TABLE 14

| Construct | Description |
|---|---|
| SB03498 | L354I |
| SB03505 | L391V |
| SB03383 | Q414E |
| SB03375 | L409V |
| SB03393 | S463P |
| SB03377 | L384M |
| SB03511 | L354I + L384M |

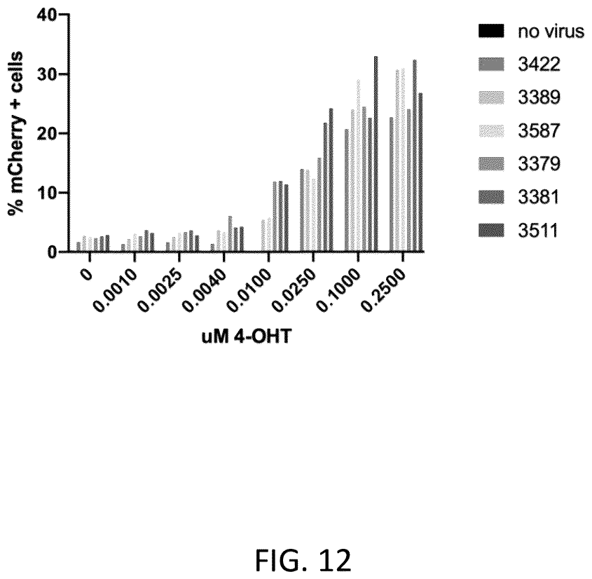

As shown in FIG. 12, the second transduction screen confirmed improved 4-OHT response for mutants identified in a transfection screen from Example 2. In particular, the mutants M517A, and N413D, and L354I+L384M demonstrated an improved 4-OHT response as compared to wild-type ERT2 (construct 3422). Notably, the improved 4-OHT response of the L354I+L384M mutant was confirmed in both the first and the second transduction screens. ERT2 mutants demonstrating improved 4-OHT binding in the second transduction screen are shown in Table 15.

TABLE 15

| Construct | Description |
|---|---|
| SB03379 | M517A |
| SB03381 | N413D |
| SB03511 | L354I + L384M |

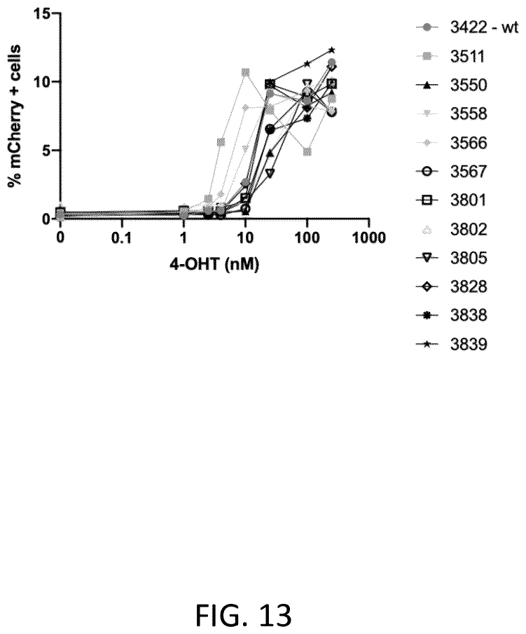

As shown in FIG. 13, the third transduction screen confirmed improved 4-OHT response for mutants identified in a transfection screen from Example 2. In particular, the mutants 1524L, M421L, and L354I+L384M demonstrated an improved 4-OHT response as compared to wild-type ERT2 (construct 3422). ERT2 mutants demonstrating improved 4-OHT binding in the third transduction screen are shown in Table 16.

TABLE 16

| Construct | Description |
|---|---|
| SB03558 | I524L |
| SB03566 | M421L |
| SB03511 | L354I + L384M |

### Example 4

### Modified ER-LBD Library Screen for Assessing Sensitivity of ER-LBD Domains to Ligands

### Materials and Methods

### Modified ER-LBD Combinatorial Library Screen

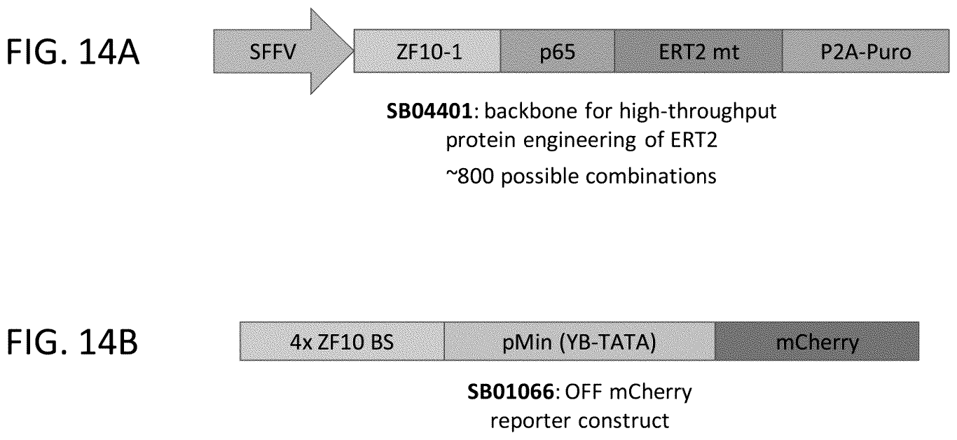

HEK293T cells were transduced with SB04401, a combinatorial ERT2 library (FIG. 14A) comprised of ˜800 unique ER-LBD variants, each of which are a unique combination of the substitutions given in Table 18 along with rationale for their inclusion. To generate a homogenous cell line where each cell had a unique ER-LBD variant per cell, viral integration of transduced HEK293T cells were quantified by copy number assay. A cell line with an average viral integration of <1 copy of the ERT2 mutant library per cell was identified and subjected to puromycin selection. The selected cell line was then transduced with a SB01066 mCherry reporter (FIG. 14B). Transduced cells were then tested for sensitivity to endoxifen and 4-OHT via the mCherry reporter which will express if an ER-LBD variant is sensitive to tested concentrations as low as about 0.1 nM up to about 1 uM. Cells expressing mCherry and therefor responsive to treatment of endoxifen were sorted followed by isolation of genomic DNA from said sorted cells. Variants were identified from the isolated genomic DNA by PCR amplification of the ERT2 coding sequence followed by insertion of the PCR product into pcr4 TOPO vector (Life Technologies). Colonies obtained were then submitted for Sanger Sequencing. Identified mutants were cloned into constructs, e.g., SB06136-SB06153 (ZF10-1_p65_ERT2 mutant with a modified ER-LBD).

TABLE 17

| Mutation | Rationale |
|---|---|
| L391V | Set 2: improve the affinity of 4-OHT/endoxifen |
| | to ERT2, single point mutation in the |
| | ligand binding pocket |
| L409V | Set 1: improve the affinity of 4-OHT, |
| | mutation in the ligand binding pocket, |
| | selected based on a substitution present |
| | in an ER homologue, increased binding |
| | to estradiol |
| Q414E | Set 1: improve the affinity of 4-OHT, |
| | mutation in the ligand binding pocket, |
| | selected based on a substitution present |
| | in an ER homologue |
| N413D | Set 1: improve the affinity of 4-OHT, |
| | mutation in the ligand binding pocket, |
| | selected based on a substitution present |
| | in an ER homologue |
| S463P | Negative control, clinical mutation |
| M421L | Set 1: improve the affinity of 4-OHT, |
| | mutation in the ligand binding pocket, |
| | selected based on a substitution present |
| | in an ER homologue |
| M517A | Set 1: mutation in the ligand binding |
| | pocket, selected based on a substitution |
| | present in an ER homologue, binds to |
| | Estradiol with ~1 kcal/mol (in |
| | comparison 4-OHT binding is disfavoured |
| | by ~1.5 kcal/mol) |
| L354I | Set 2: improve the affinity of |
| | 4-OHT/endoxifen to ERT2, single point |
| | mutation in the ligand binding pocket |
| L384M | Set 1: improve the affinity of 4-OHT, |
| | mutation in the ligand binding pocket, |
| | selected based on a substitution present |
| | in an ER homologue |
| H524L | Set 5: rational optimization of ERT2 |
| | to improve its binding to 4-OHT and |
| | Endoxifen, focused on MD simulations |
| | in the ligand binding pocket |

Modified ER-LBD Validation in U87MG with mCherry Reporter

About 16 hours before transduction 100k U87MG: 1066 cells were seeded into 16 wells in 12 well plate format. During transduction, cells in each well were transduced with 100k pg of virus of ERT2 variant constructs. After transduction, cells were split into 50k cells per well in a 24 well plate format, and treated with drug-free media or a range of endoxifen or 4-OHT drug conditions. About two days after treatment of cells with endoxifen or 4-OHT, cells were collected and mCherry reporter expression was quantified by flow cytometry.

### Results

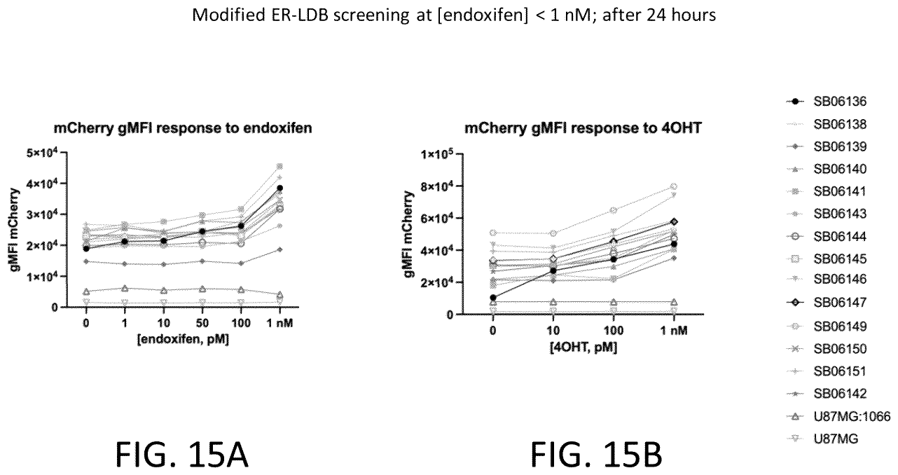

Screening of the modified ER-LBD construct library identified a subset of ERT2 variants that were sensitive to endoxifen at 1 nM. Among this subset, 15 variants (Table 19) were validated in U87MG cells for their ability to activate an mCherry reporter (SB01066; FIG. 14B) at a range of concentrations of 0 pM, 10 pM, 50 pM, 100 pM, and 1 nM of endoxifen (FIG. 15A) and 4-OHT (FIG. 15B). Expression of mCherry was quantified about 24 hours after treatment with endoxifen or 4-OHT. As demonstrated, all 15 variants were sensitive to endoxifen and 4-OHT at concentrations of about 1 nM or less, whereas negative controls U87MG and U87MG: 1066 negative controls showed no sensitivity.

TABLE 19

| Construct | Description |
|---|---|
| SB06136 | ZF10-1_p65_ERT2_mutant 81 |
| SB06138 | ZF10-1_p65_ERT2_mutant 93 |
| SB06139 | ZF10-1_p65_ERT2_mutant 86 |
| SB06140 | ZF10-1_p65_ERT2_mutant 95 |
| SB06141 | ZF10-1_p65_ERT2_mutant 88 |
| SB06142 | ZF10-1_p65_ERT2_mutant 77 |
| SB06143 | ZF10-1_p65_ERT2_mutant 49 |
| SB06144 | ZF10-1_p65_ERT2_mutant 58 |
| SB06145 | ZF10-1_p65_ERT2_mutant 62 |
| SB06146 | ZF10-1_p65_ERT2_mutant 63 |
| SB06147 | ZF10-1_p65_ERT2_mutant 55 |
| SB06149 | ZF10-1_p65_ERT2_mutant 41 |
| SB06150 | ZF10-1_p65_ERT2_mutant 43 |
| SB06151 | ZF10-1_p65_ERT2_mutant 46 |
| SB06152 | ZF10-1_p65_ERT2_mutant 40 |

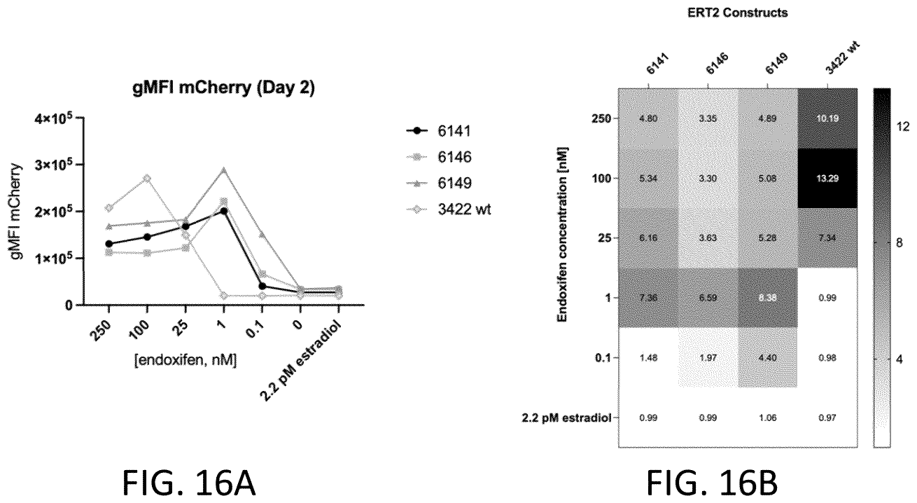

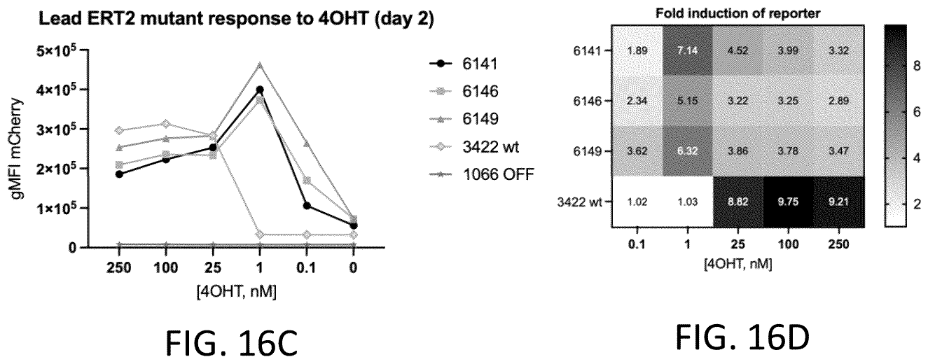

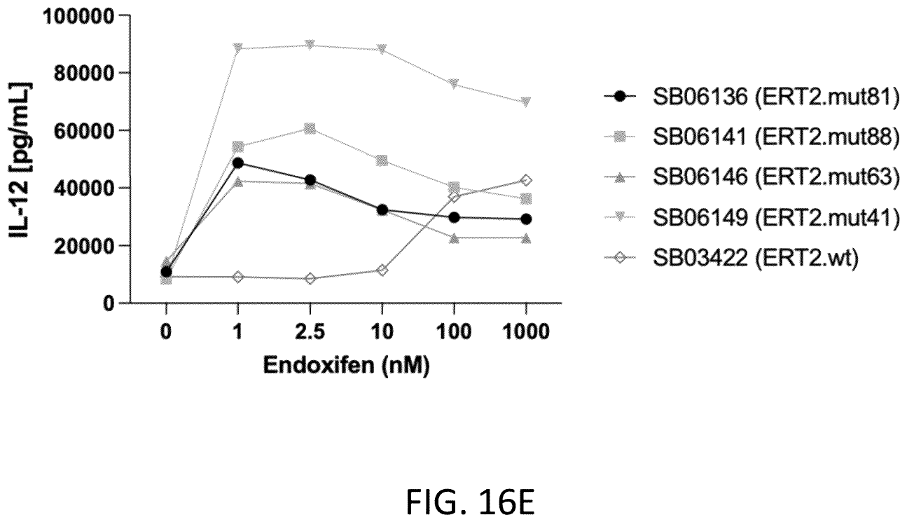

Select ERT2 variants (Table 20) from the previous screens were further tested for sensitivity to endoxifen and 4-OHT, compared to wild-type ERT2. Tests showed that activation of wild-type ERT2 begins at about 25 nM endoxifen and at about 25 nM 4-OHT, while three ERT2 variants tested activate mCherry expression at 1 nM and 0.1 nM endoxifen and at 1 nM and 0.1 nM 4-OHT (FIG. 16A-16D). Exemplary heat maps show fold activation of mCherry expression of constructs tested at various concentrations, including 2.2 pM estradiol (FIG. 16B). ERT2 is ER-alpha mutated to be insensitive to estradiol, the lead ERT2 variants were confirmed to continue to be insensitive to biologically observed concentrations of estradiol. Fold activation was calculated by gMFI mCherry levels of each test condition divided by gMFI mCherry levels of U87MG cells transduced with the reporter (SB01066).

Modified ER-LBD Validation in U87MG with IL-12 Reporter

In another experiment, a U87MG cell line with an IL-12 reporter construct (ZF10-1 BS_pMinYBTATA: IL-12) was transduced with modified ER-LBD constructs SB06136, SB06141, SB06146, SB06149, or unmodified ERT2 construct SB03422 at 1 pg/cell of virus.

Next day transduced cells were split into wells at 100k cells/well and treated with indicated drug concentration of Endoxifen in a volume of 200 uL media. After 48 hours of drug treatment, supernatants were harvested and quantified for IL-12 expression via ELISA using standard techniques.

As shown in FIG. 16E, constructs with the modified ERT2-LBD also demonstrated improved sensitivity to tamoxifen metabolites compared to WT (empty circle), as assessed by IL-12 secretion. Additional experiments further indicated that the modified ER-LBD constructs comprising the additional amino acid substitutions L384M/L391V/N413D/M421L/S463P/H524L (ERT2_mutant 81) in a transcriptional reporter assay for dimerization activity did not directly alter dimerization of two modified ER-LBDs to 4-OHT, suggesting dimerization itself was comparable to unmodified ERT2 (data not shown), consistent with substitution site selection based on their predicted impact on binding to tamoxifen metabolites.

TABLE 20

| Construct | Description |
|---|---|
| SB06141 | ZF10-1_p65_ERT2_mutant 88 |
| SB06146 | ZF10-1_p65_ERT2_mutant 63 |
| SB06149 | ZF10-1_p65_ERT2_mutant 41 |
| SB03422 | ZF10-1_p65_ERT2 wild-type |
| SB01066 | pMinYBTATA: mCherry reporter |

### Example 5

### ERT2 Mutant Validation in NK Cells

### Materials and Methods

Validation ERT2 Mutants with mCherry Reporter in NK Cells

After 10 days of feeder cell activation, NK cells were co-transduced with ERT2 mutant virus and reporter SB01066 virus (Experimental Set-up 1). On Day 2 after transduction, transduced NK cells were treated with endoxifen or 4-OHT at a range of concentrations of 0 nM, 0.01 nM, 0.1 nM, 1 nM, and 10 nM. On Day 4, cells were checked for mCherry expression by flow cytometry.

Experimental Set-Up 1
Validation ERT2 Mutants with IL12 Reporter in NK Cells

After 10 days of feeder cell activation, NK cells were transduced with ERT2/IL12 vectors (Table 22; Experimental Set-up 2). On Day 2 after transduction, transduced NK cells were treated with endoxifen at a range of concentrations of 0 nM, 0.1 nM, 1 nM, 10 nM, 100 nM, and 1000 nM. On Day 4, cells were checked for IL-12 expression via Luminex.

Experimental Set-Up 2

### Results

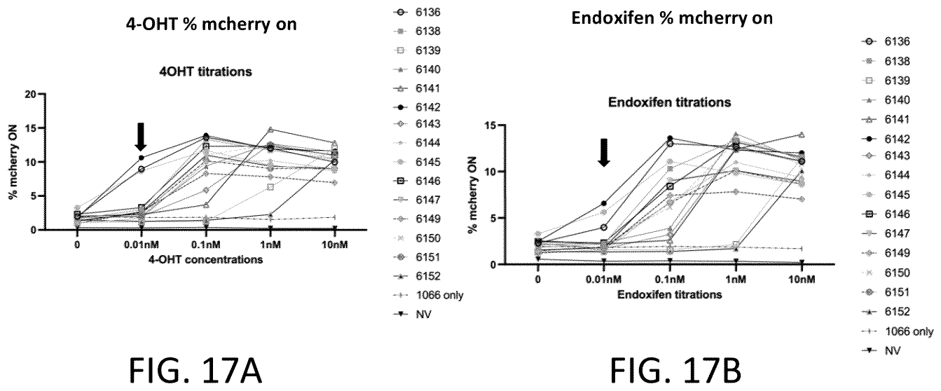

Select ERT2 variants (Table 21; FIG. 17A-17B) from the previous screens were further tested for sensitivity to endoxifen and 4-OHT in primary NK cells. Among the variants tested, SB06142 (mutant 77; L354I/L391V/N413D/M421L/S463P/M517A/H524L), SB06136 (mutant 81; L384M/L391V/N413D/M421L/S463P/H524L), and SB06145 (mutant 62; LA09V/N413D/Q414E/M421L/S463P/H524L) were sensitive to both endoxifen and 4-OHT at concentrations of 0.1 nM (FIG. 17A-17B).

TABLE 21

| Construct | Description |
|---|---|
| SB06136 | ZF10-1_p65_ERT2_mutant 81 |
| SB06138 | ZF10-1_p65_ERT2_mutant 93 |
| SB06139 | ZF10-1_p65_ERT2_mutant 86 |
| SB06140 | ZF10-1_p65_ERT2_mutant 95 |
| SB06141 | ZF10-1_p65_ERT2_mutant 88 |
| SB06142 | ZF10-1_p65_ERT2_mutant 77 |
| SB06143 | ZF10-1_p65_ERT2_mutant 49 |
| SB06144 | ZF10-1_p65_ERT2_mutant 58 |
| SB06145 | ZF10-1_p65_ERT2_mutant 62 |
| SB06146 | ZF10-1_p65_ERT2_mutant 63 |
| SB06147 | ZF10-1_p65_ERT2_mutant 55 |
| SB06149 | ZF10-1_p65_ERT2_mutant 41 |
| SB06150 | ZF10-1_p65_ERT2_mutant 43 |
| SB06151 | ZF10-1_p65_ERT2_mutant 46 |
| SB06152 | ZF10-1_p65_ERT2_mutant 40 |

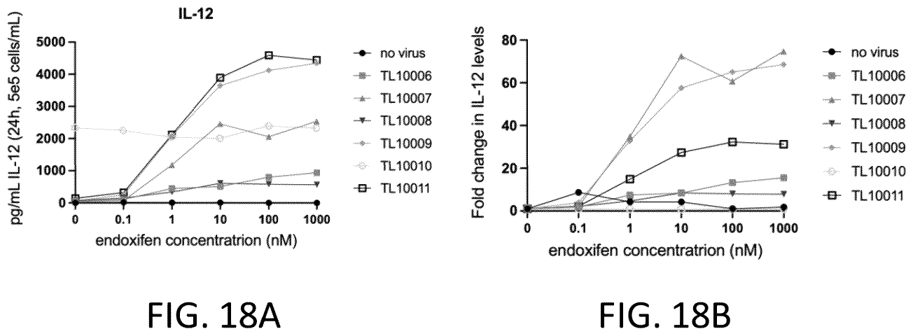

To demonstrate delivery of a therapeutic polypeptide, ERT2/IL-12 vectors were constructed as shown in Table 22, and tested for sensitivity to endoxifen in primary NK cells. Testing of ERT2/IL-12 vectors in NK cells showed that TL10009, with ERT2-L354I/L391V/N413D/S463P/H524L from SB06142 and crIL12 CD16 CS, shows best induction and fold change in IL-12 (FIG. 18A-18B).

TABLE 22

| | | Inducible | | | | | |
|---|---|---|---|---|---|---|---|
| | | IL-12 | | | | | |
| | | (driven by | | | ZF DNA | | |
| | | 4x ZF5BS | | Promoter: | Binding | | ERT2 |
| TL# | SB# | YBTATA) | Insulator | ZF | Domain | Activator | mutant |
| TL10006 | SB07123 | ZFN_YB- | A2 | SFFV: 5-7 | ZF5-7 | P65 | Mutant |
| | | TATA-sIL- | | | | | 81 |
| | | 12 1x | | | | | |
| | | AUSLDE | | | | | |
| TL10007 | SB07127 | ZFN_YB- | A2 | SV40: 5-7 | ZF5-7 | P65 | Mutant |
| | | TATA | | | | | 81 |
| | | IL12_CD16 | | | | | |
| | | TACE | | | | | |
| | | cleavage | | | | | |
| | | site_B7-1 | | | | | |
| | | TM | | | | | |
| TL10008 | SB07129 | ZFN_YB- | A2 | SFFV: 5-7 | ZF5-7 | P65 | Mutant |
| | | TATA-sIL- | | | | | 77 |
| | | 12 1x | | | | | |
| | | AUSLDE | | | | | |
| TL10009 | SB07133 | ZFN_YB- | A2 | SV40: 5-7 | ZF5-7 | P65 | Mutant |
| | | TATA | | | | | 77 |
| | | IL12_CD16 | | | | | |
| | | TACE | | | | | |
| | | cleavage | | | | | |
| | | site_B7-1 | | | | | |
| | | TM | | | | | |
| TL10010 | SB07135 | ZFN_YB- | A2 | SFFV: 5-7 | ZF5-7 | P65 | Mutant |
| | | TATA-sIL- | | | | | 62 |
| | | 12 1x | | | | | |
| | | AUSLDE | | | | | |
| TL10011 | SB07139 | ZFN_YB- | A2 | SV40: 5-7 | ZF5-7 | P65 | Mutant |
| | | TATA | | | | | 62 |
| | | IL12_CD16 | | | | | |
| | | TACE | | | | | |
| | | cleavage | | | | | |
| | | site_B7-1 | | | | | |
| | | TM | | | | | |

### Example 6

### Assessment of Modified ER-LBDS in Induced Cell Death Systems

### Suicide-Switch Assay

A 24-well dish was seeded with 25,000 Lent-X HEK293T cells (Takara, Catalog #: 632180). The next day, the cells were transduced with 25,000 pg of virus for constructs SB07351-SB07354: ERT2mut-casp9 constructs tagged with mCherry to track successfully transduced cells in a population. The constructs each take the form ERT2*mut-Casp9-IRES-RFP (SB07351=mut81; SB07352=mut88; SB07353=mut63; SB07354=mut41). The next day, transduced cells were split into a 96-well plate with ˜5,000 cells in each well and loaded into an incucyte to quantify cell expansion via images every 2 hours detecting mCherry expression. Media was exchanged 24 hours later with new media with indicated concentration of 4-OHT or no drug added. Cells continued to be imaged by the incucyte every 2 hours for 48 hours post exchange of media.

Cell death was quantified via detection of suicide-switch linked mCherry expression using the following formula:

Killing efficiency=100−((mCherry expression in drug treated population)/(mCherry expression in ND population)) [NOTE: mCherry background prevents 100% KE from being calculated; >95% killing efficiency is considered complete killing in this assay]

### Suicide-Switch Assay Results

The modified ER-LBD variants identified above were assessed in a transcriptional reporter assay (see Example 2 and Example 3). However, as illustrated in FIG. 19, the mechanism of an ER-mediated transcriptional activation generally involves nuclear translocation (FIG. 19, top panel). In contrast, ER-mediated suicide-switch induction generally involves dimerization within the cytoplasm (FIG. 19, bottom panel). Accordingly, the improved sensitivity in the transcriptional activation would not generally predict efficacy and sensitivity for ER-mediated suicide-switch induction given the different mechanisms of action. Thus, modified ER-LBD variants identified above were further assessed in suicide-switch system.

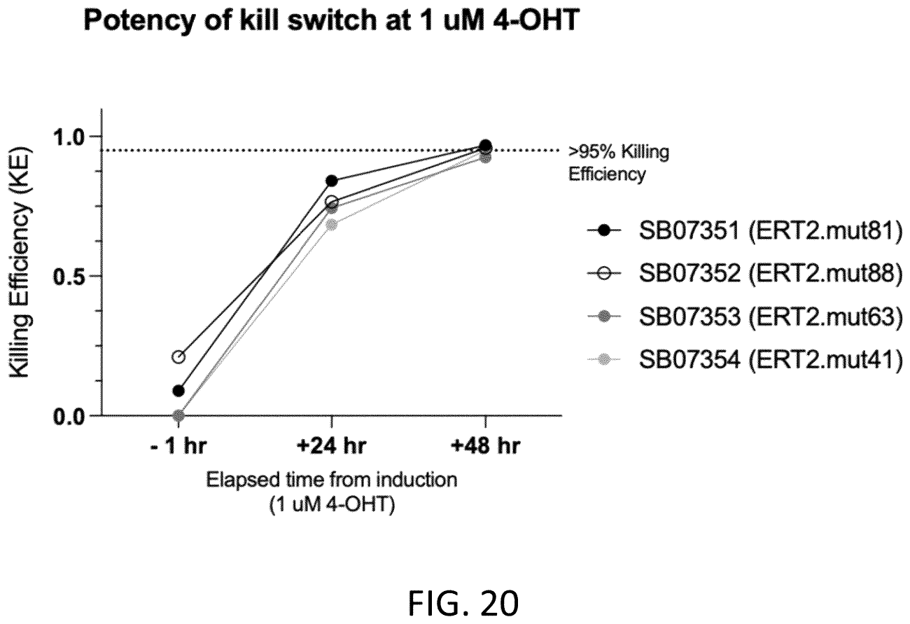

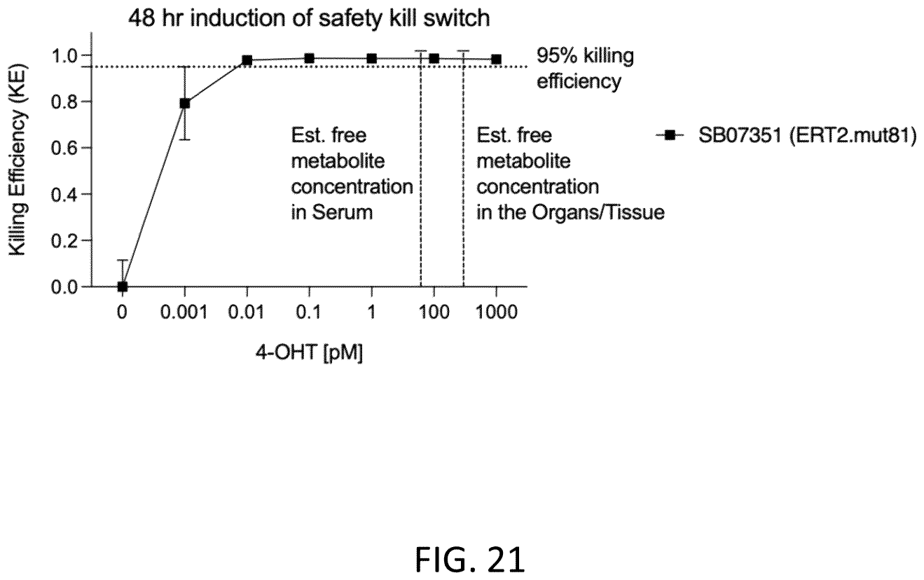

As shown in FIG. 20, suicide-switch constructs with the modified ER-LBD variants achieved potent killing (>95%) in HEK293T cells at 1 μM 4-OHT over a period of 48 hours. As shown in FIG. 21, a suicide-switch construct with the modified ER-LBD variant “mut81” achieved potent killing (>95%) in HEK293T cells down to 0.01 pM of 4-OHT and robust killing (˜80%) at 0.001 pM of 4-OHT. Notably, efficient killing occurred at a concentration 400× lower than the Ctrough of 4-OHT in humans taking FDA approved daily dose of Tamoxifen (see Binkhorst et al. [Breast Cancer Research and Treatment volume 152, pages 119-128 (2015)] and Hussaarts et al [Cancers (Basel). 2019 March; 11 (3): 403.]).

### Example 7

### Further Assessment of Modified ER-LBDS in Induced Cell Death Systems

Modified ER-LBD variants described herein are assessed as in the 293T suicide-switch killing assay of Example 6. Included in the assessment are ERT2mut-casp9 constructs SB07351-SB07354 constructs described in Example 6. The modified ER-LBD variants are assessed against the unmodified ERT2 across a at a range of endoxifen concentrations, combinations of tamoxifen metabolites, and estradiol. Conditions include 0, 0.0001, 0.001, 0.01, 0.1, 1.0, 10, 100, 1000, 2.5 nM & 0.4 nM Endoxifen or 4-OHT, 0.25 nM and 0.04 nM Endoxifen and 4-OHT, and 2.2 pM Estradiol.

What is assessed includes:
1. Minimum concentration of endoxifen that achieves complete killing with the ERT2 mutants and wt ERT2 suicide switches
2. Killing efficiency of mutant and wt ERT2 suicide switches at pharmacological concentrations of tamoxifen metabolites estimated in the serum (0.25 nM Endoxifen and 0.04 nM 4-OHT) and in organs/tissue/tumor (2.5 nM Endoxifen& 0.4 nM 4-OHT); and
3. Continued insensitivity to 2.2 pM of estradiol (minimum level of estradiol observed in a cohort of women at the lowest point in their menstrual cycle).

### Example 8

### Validation of an Exemplary ERT2 Mutant in Inducible Cell Death System in T Cells

Primary T cells isolated from a donor were thawed and stimulated with Dynabeads for 24 hours. Next, ERT2*mut81-Casp9-IRES-RFP (SB07351, prepared as lentivirus) was transduced into the T cells. Two days post transduction, cell media were replaced with T Cell media containing IL-2 to promote expansion. 1 week post transduction, cells were quantified for transduction efficiency (Safety kill switch transduction was tracked by expression of RFP tag) by flow cytometry and then loaded into incucyte for quantification of killing efficiency.

### Killing Assay:

D0-D1: Expansion of cells in drug free T cell media (with IL-2)
D1-onward: Drug free T cell media (with IL-2) was exchanged with media containing indicated drug conditions (as well as IL-2)
Killing efficiency was calculated by the following formula:

1−(RFP expression of cells in drug treated conditions)/(RFP expression of transduced cells in the No Drug condition)

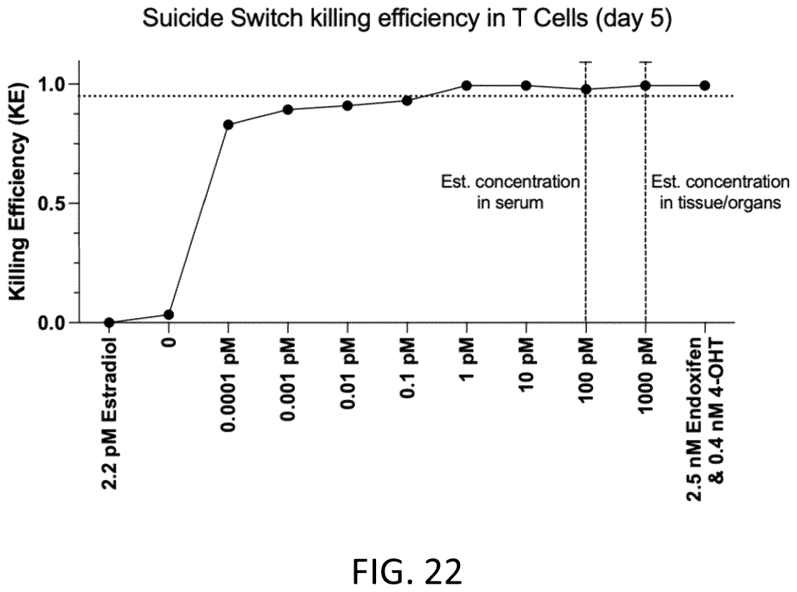

FIG. 22 shows killing efficiency of the safety kill switch by day 5 of treatment with indicated drug conditions. The engineered kill switch maintained its insensitivity to Estradiol at 2.2 pM (maximum level of estradiol estimated in women). Strong killing efficiency was demonstrated by day 5 at endoxifen concentrations as low at 0.001 pM, and complete killing efficiency (>=95%) was demonstrated by day 5 at 1 pM and higher. Thus, the engineered safety kill switch ERT2*mut81-Casp9 achieved potent induced cell killing in primary T cells at ligand concentrations that would be pharmacologically relevant in a tumor/tissue/organ environment.

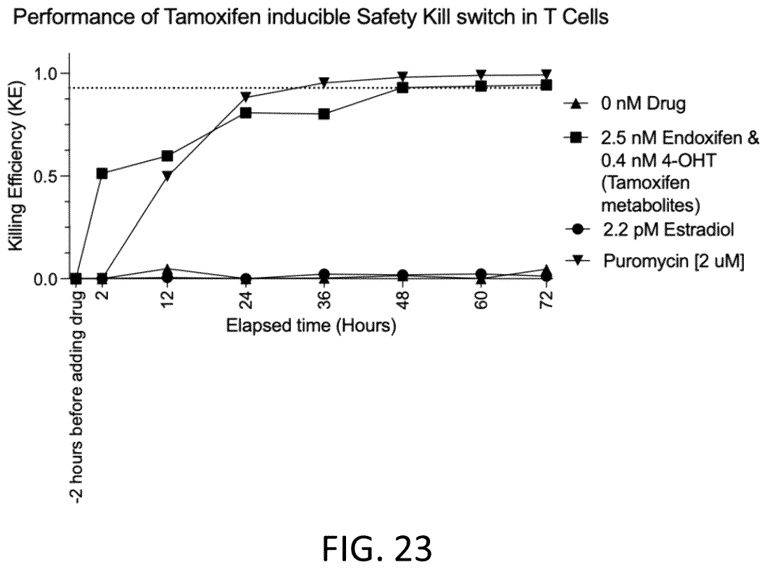

FIG. 23 shows killing efficiency of the safety kill switch over time at the indicated drug conditions. The elapsed hours are from time elapsed from adding drug. As shown, the safety kill switch was insensitive to 2.2 pM Estradiol, and did not induce cell killing in the absence of the small molecule inducer cocktail (exemplified here as 2.5 nM endoxifen and 0.4 nM 4-OHT. Also as shown, complete killing was observed within 48 hours when stimulated with tamoxifen metabolites (2.5 nM Endoxifen & 0.4 nM 4-OHT). As a positive control for complete killing, T cells transduced with a construct expressing RFP were treated with puromycin.

Taken together, the results show that the engineered safety kill switch ERT2*mut81-Casp9 achieved potent induced cell killing in primary T cells at pharmacologically relevant ligand concentrations in tumor/tissue/organ environment, had minimal to no killing activity in the absence of the non-endogenous ligands, and maintained insensitivity to the endogenous hormone estradiol at 2.2 pM (the highest concentration observed in women at peak hormone cycle; much lower concentrations observed in men).

### Example 9

### Time Course of Exemplary ERT2 Mutant Safety Switch Activity in HEK Cells

500k HEK293T cells were transduced with the following constructs at a MOI of one: SB07351, SB07352, SB07353, SB07354, SB02956. The constructs had the following architecture: pSFFV: ERT2-iCasp9-IRES-RFP. (SB07351=mut81; SB07352=mut88; =mut63; SB07354-mut41, SB02956=WT ERT2)

Twenty-four hours later, ˜3k transduced cells were plated into each well of a 96w plate and cultured in 100 μL of drug-free growth media (DMEM+10% FBS, 1% p-s, 1% Glutamax). 16 hours after loading the plate in the Incuyte, media was exchanged with fresh drug-free media or with media containing the following drug conditions for an elapsed time course: 0.25 nM endoxifen and 0.04 nM 4-OHT (serum concentrations of tamoxifen metabolites); 2.5 nM endoxifen+0.4 nM 4-OHT (concentration of tamoxifen metabolites in brain), 2.5 nM estradiol (an ultra-high concentration that exceeds physiological relevance), and no drug control.

Killing efficiency was calculated by taking the average mCherry area quantified by the incucyte in the drug treated sample divided by the mCherry area quantified in the ND conditions of the same transduced population and subtracting that fraction from 1. 95% KE was defined as complete killing by previous studies with this same mCherry reporter being treated with Puromycin, a toxic drug that results in complete killing of HEK293T cells within 48 hours of application.

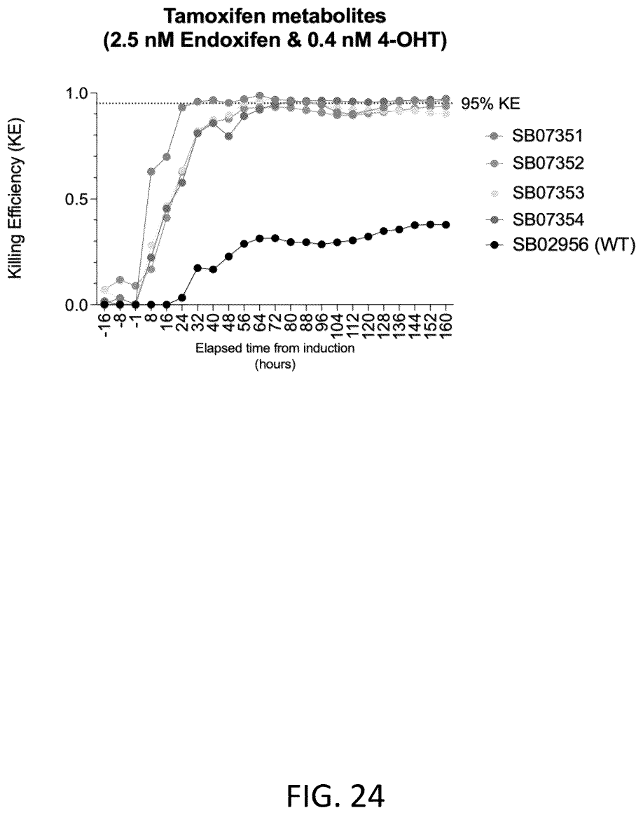

FIG. 24 shows time course results for the indicated drug conditions. Results suggest that all four ERT2 mutant safety switches (mut81, mut 88, mut63, and mut41) achieved greater killing efficiency, and at a faster rate as compared to the WT ERT2 safety switch.

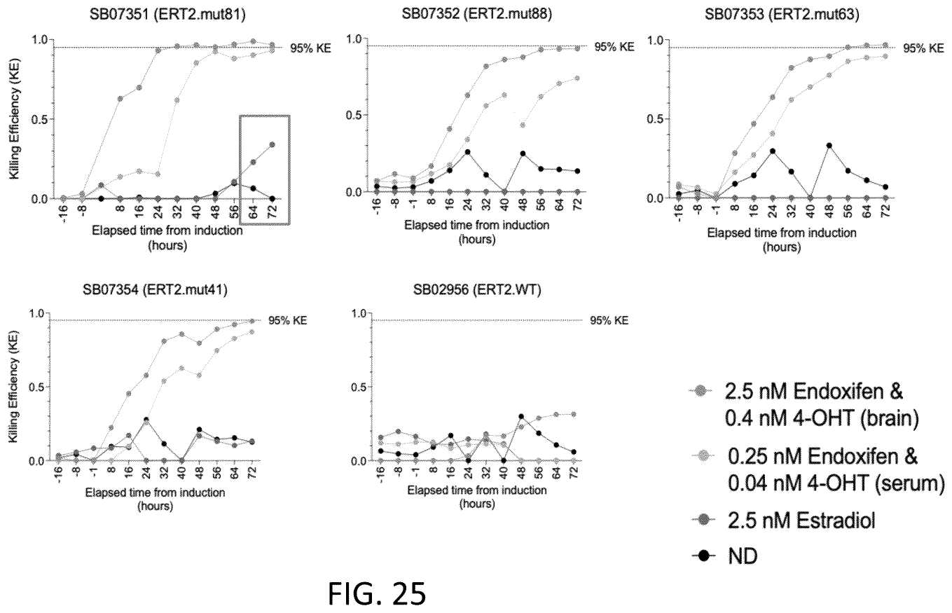

FIG. 25 shows time course results for the indicated drug conditions. Results suggest that the majority of switches maintained insensitivity to 2.5 nM estradiol even with long exposure times. SB07351 showed modest activity at late time points of exposure to the ultra-high estradiol concentration of 2.5 nM.

### Example 10

### Evaluation of Exemplary ERT2 Mutant Transcriptional Switch Activity

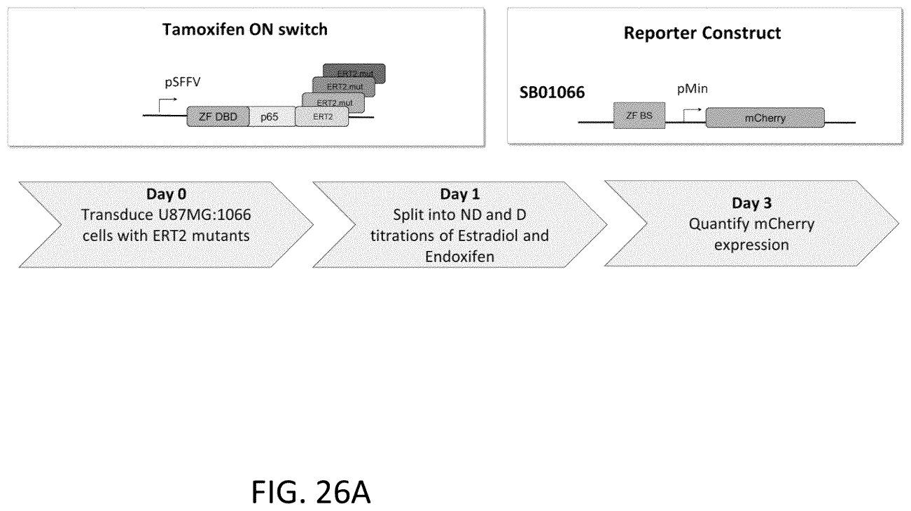

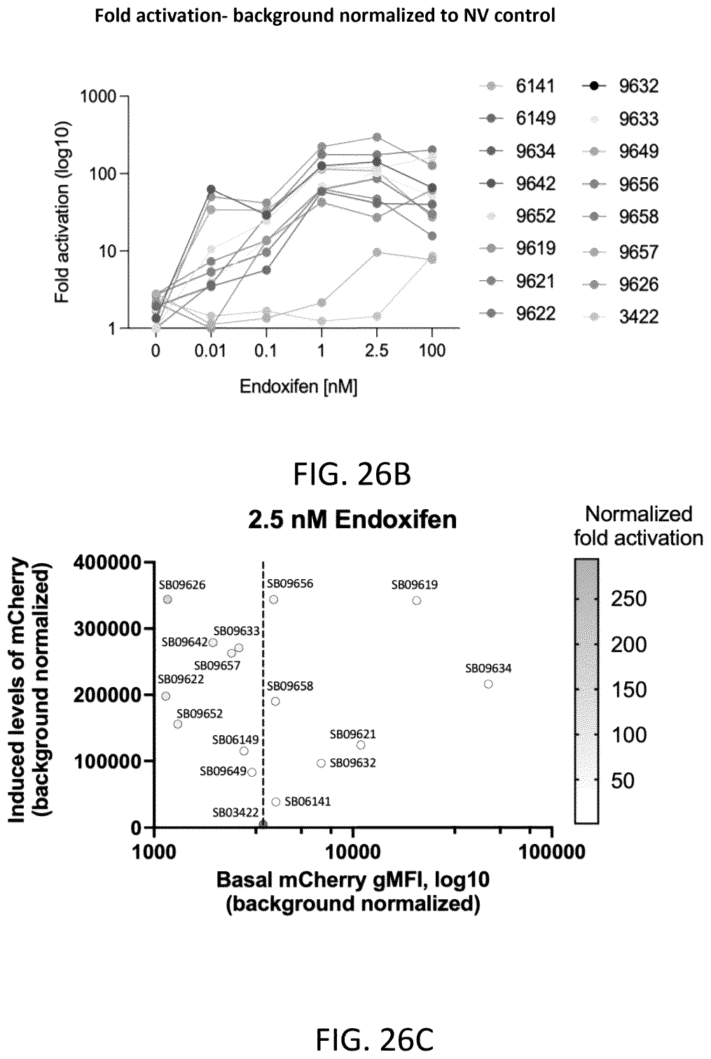

FIG. 26A depicts constructs and experimental methods for evaluating exemplary ERT2 mutant transcriptional switches. U87MG cells were transduced with the mCherry reporter construct SB01066, comprising synthetic promoter comprising a 4×ZF-10 zinc finger binding site (SEQ ID NO: 59) linked to a minimal promoter operably linked to an mCherry encoding sequence. This generated a U87MG: 1066 reporter cell line used for assessing transcriptional switch activity of ERT2 transcriptional switch constructs.

25k U87MG: 1066 reporter transduced line was transduced at a MOI of 1 with various ERT2 transcriptional switch constructs having the following structure: pSFFV: ZF10-1 (zinc finger DNA binding domain, SEQ ID NO: 84)_TCR linker (SEQ ID NO: 86)_p65 (transcriptional activation domain, SEQ ID NO: 64)_QLCVRGSS linker (SEQ ID NO: 88)_ERT2 domain. The ERT2 mutants included a number of amino acid substitutions in addition to the “WT ERT2” mutations G400V/M543A/L544A/V595A. See Table 23 below.

TABLE 23

| | | | ERT2 aa sequence |
|---|---|---|---|
| | ERT2 | | (no starting Methionine, |
| SB | mutant | | with optional A at the C- |
| Construct | ID | Mutations to ERT2 | terminal end of the domain) |
| SB09634 | ERT2. | L391V.L409V.Q414E. | SAGDMRAANLWPSPLMIKRSKKNSLALSLTAD |
| | N413D. | N413D.S463P.M517A. | QMVSALLDAEPPILYSEYDPTRPFSEASMMGL |
| | S463P. | H524L | LTNLADRELVHMINWAKRVPGFVDLTLHDQVH |
| | H524L. | | LLECAWLEILMIGVVWRSMEHPVKLLFAPNLV |
| | mutant. | | LDRDEGKCVEGMVEIFDMLLATSSRFRMMNLQ |
| | 25 | | GEEFVCLKSIILLNSGVYTFLPSTLKSLEEKD |
| | | | HIHRVLDKITDTLIHLMAKAGLTLQQQHQRLA |
| | | | QLLLILSHIRHASNKGMELLYSMKCKNVVPLY |
| | | | DLLLEAADAHRLHAPTSRGGASVEETDQSHLA |
| | | | TAGSTSSHSLQKYYITGEAEGFPATA (SEQ |
| | | | ID NO: 90) |
| SB09642 | ERT2. | L409V.N413D.S463P. | SAGDMRAANLWPSPLMIKRSKKNSLALSLTAD |
| | N413D. | M421L.L384M.H524L | QMVSALLDAEPPILYSEYDPTRPFSEASMMGL |
| | S463P. | | LTNLADRELVHMINWAKRVPGFVDLTLHDQVH |
| | H524L. | | LLECAWMEILMIGLVWRSMEHPVKLLFAPNLV |
| | mutant. | | LDRDQGKCVEGLVEIFDMLLATSSRFRMMNLQ |
| | 14 | | GEEFVCLKSIILLNSGVYTFLPSTLKSLEEKD |
| | | | HIHRVLDKITDTLIHLMAKAGLTLQQQHQRLA |
| | | | QLLLILSHIRHMSNKGMELLYSMKCKNVVPLY |
| | | | DLLLEAADAHRLHAPTSRGGASVEETDQSHLA |
| | | | TAGSTSSHSLQKYYITGEAEGFPATA (SEQ |
| | | | ID NO: 91) |
| SB09652 | ERT2. | L391V.N413D.S463P. | SAGDMRAANLWPSPLMIKRSKKNSLALSLTAD |
| | N413D. | M517A.M421L.L354I. | QMVSALLDAEPPILYSEYDPTRPFSEASMMGL |
| | S463P. | H524L | LTNLADREIVHMINWAKRVPGFVDLTLHDQVH |
| | H524L. | | LLECAWLEILMIGVVWRSMEHPVKLLFAPNLL |
| | mutant. | | LDRDQGKCVEGLVEIFDMLLATSSRFRMMNLQ |
| | 37 | | GEEFVCLKSIILLNSGVYTFLPSTLKSLEEKD |
| | | | HIHRVLDKITDTLIHLMAKAGLTLQQQHQRLA |
| | | | QLLLILSHIRHASNKGMELLYSMKCKNVVPLY |
| | | | DLLLEAADAHRLHAPTSRGGASVEETDQSHLA |
| | | | TAGSTSSHSLQKYYITGEAEGFPATA (SEQ |
| | | | ID NO: 102) |
| SB09619 | ERT2. | L391V.L409V.N413D. | SAGDMRAANLWPSPLMIKRSKKNSLALSLTAD |
| | N413D. | S463P.M517A.M421L. | QMVSALLDAEPPILYSEYDPTRPFSEASMMGL |
| | S463P. | L354I.H524L | LTNLADREIVHMINWAKRVPGFVDLTLHDQVH |
| | H524L. | | LLECAWLEILMIGVVWRSMEHPVKLLFAPNLV |
| | mutant. | | LDRDQGKCVEGLVEIFDMLLATSSRFRMMNLQ |
| | 41 | | GEEFVCLKSIILLNSGVYTFLPSTLKSLEEKD |
| | | | HIHRVLDKITDTLIHLMAKAGLTLQQQHQRLA |
| | | | QLLLILSHIRHASNKGMELLYSMKCKNVVPLY |
| | | | DLLLEAADAHRLHAPTSRGGASVEETDQSHLA |
| | | | TAGSTSSHSLQKYYITGEAEGFPATA (SEQ |
| | | | ID NO: 92) |
| SB09621 | ERT2. | L391V.Q414E.N413D. | SAGDMRAANLWPSPLMIKRSKKNSLALSLTAD |
| | N413D. | S463P.M421L.L354I. | QMVSALLDAEPPILYSEYDPTRPFSEASMMGL |
| | S463P. | L384M.H524L | LTNLADREIVHMINWAKRVPGFVDLTLHDQVH |
| | H524L. | | LLECAWMEILMIGVVWRSMEHPVKLLFAPNLL |
| | mutant. | | LDRDEGKCVEGLVEIFDMLLATSSRFRMMNLQ |
| | 51 | | GEEFVCLKSIILLNSGVYTFLPSTLKSLEEKD |
| | | | HIHRVLDKITDTLIHLMAKAGLTLQQQHQRLA |
| | | | QLLLILSHIRHMSNKGMELLYSMKCKNVVPLY |
| | | | DLLLEAADAHRLHAPTSRGGASVEETDQSHLA |
| | | | TAGSTSSHSLQKYYITGEAEGFPATA (SEQ |
| | | | ID NO: 93) |
| SB09622 | ERT2. | L391V.L409V.N413D. | SAGDMRAANLWPSPLMIKRSKKNSLALSLTAD |
| | N413D. | S463P.M517A.M421L. | QMVSALLDAEPPILYSEYDPTRPFSEASMMGL |
| | S463P. | H524L | LTNLADRELVHMINWAKRVPGFVDLTLHDQVH |
| | H524L. | | LLECAWLEILMIGVVWRSMEHPVKLLFAPNLV |
| | mutant. | | LDRDQGKCVEGLVEIFDMLLATSSRFRMMNLQ |
| | 17 | | GEEFVCLKSIILLNSGVYTFLPSTLKSLEEKD |
| | | | HIHRVLDKITDTLIHLMAKAGLTLQQQHQRLA |
| | | | QLLLILSHIRHASNKGMELLYSMKCKNVVPLY |
| | | | DLLLEAADAHRLHAPTSRGGASVEETDQSHLA |
| | | | TAGSTSSHSLQKYYITGEAEGFPATA (SEQ |
| | | | ID NO: 94) |
| SB09632 | ERT2. | L391V.L409V.Q414E. | SAGDMRAANLWPSPLMIKRSKKNSLALSLTAD |
| | mutant. | N413D.S463P.L354I. | QMVSALLDAEPPILYSEYDPTRPFSEASMMGL |
| | N413D. | H524L | LTNLADREIVHMINWAKRVPGFVDLTLHDQVH |
| | S463P. | | LLECAWLEILMIGVVWRSMEHPVKLLFAPNLV |
| | H524L. | | LDRDEGKCVEGMVEIFDMLLATSSRFRMMNLQ |
| | 36 | | GEEFVCLKSIILLNSGVYTFLPSTLKSLEEKD |
| | | | HIHRVLDKITDTLIHLMAKAGLTLQQQHQRLA |
| | | | QLLLILSHIRHMSNKGMELLYSMKCKNVVPLY |
| | | | DLLLEAADAHRLHAPTSRGGASVEETDQSHLA |
| | | | TAGSTSSHSLQKYYITGEAEGFPATA (SEQ |
| | | | ID NO: 95) |
| SB09633 | ERT2. | L391V.L409V.N413D. | SAGDMRAANLWPSPLMIKRSKKNSLALSLTAD |
| | mutant. | S463P.M421L.L354I. | QMVSALLDAEPPILYSEYDPTRPFSEASMMGL |
| | N413D. | L384M.H524L | LTNLADREIVHMINWAKRVPGFVDLTLHDQVH |
| | S463P. | | LLECAWMEILMIGVVWRSMEHPVKLLFAPNLV |
| | H524L. | | LDRDQGKCVEGLVEIFDMLLATSSRFRMMNLQ |
| | 66 | | GEEFVCLKSIILLNSGVYTFLPSTLKSLEEKD |
| | | | HIHRVLDKITDTLIHLMAKAGLTLQQQHQRLA |
| | | | QLLLILSHIRHMSNKGMELLYSMKCKNVVPLY |
| | | | DLLLEAADAHRLHAPTSRGGASVEETDQSHLA |
| | | | TAGSTSSHSLQKYYITGEAEGFPATA (SEQ |
| | | | ID NO: 96) |
| SB09649 | ERT2. | L391V.Q414E.N413D. | SAGDMRAANLWPSPLMIKRSKKNSLALSLTAD |
| | N413D. | S463P.M517A.M421L. | QMVSALLDAEPPILYSEYDPTRPFSEASMMGL |
| | S463P. | L354I.H524L | LTNLADREIVHMINWAKRVPGFVDLTLHDQVH |
| | H524L. | | LLECAWLEILMIGVVWRSMEHPVKLLFAPNLL |
| | mutant. | | LDRDEGKCVEGLVEIFDMLLATSSRFRMMNLQ |
| | 97 | | GEEFVCLKSIILLNSGVYTFLPSTLKSLEEKD |
| | | | HIHRVLDKITDTLIHLMAKAGLTLQQQHQRLA |
| | | | QLLLILSHIRHASNKGMELLYSMKCKNVVPLY |
| | | | DLLLEAADAHRLHAPTSRGGASVEETDQSHLA |
| | | | TAGSTSSHSLQKYYITGEAEGFPATA (SEQ |
| | | | ID NO: 97) |
| SB09656 | ERT2. | L391V.N413D.S463P. | SAGDMRAANLWPSPLMIKRSKKNSLALSLTAD |
| | mutant. | M517A.L384M.H524L | QMVSALLDAEPPILYSEYDPTRPFSEASMMGL |
| | N413D. | | LTNLADRELVHMINWAKRVPGFVDLTLHDQVH |
| | S463P. | | LLECAWMEILMIGVVWRSMEHPVKLLFAPNLL |
| | H524L. | | LDRDQGKCVEGMVEIFDMLLATSSRFRMMNLQ |
| | 128 | | GEEFVCLKSIILLNSGVYTFLPSTLKSLEEKD |
| | | | HIHRVLDKITDTLIHLMAKAGLTLQQQHQRLA |
| | | | QLLLILSHIRHASNKGMELLYSMKCKNVVPLY |
| | | | DLLLEAADAHRLHAPTSRGGASVEETDQSHLA |
| | | | TAGSTSSHSLQKYYITGEAEGFPATA (SEQ |
| | | | ID NO: 98) |
| SB09658 | ERT2. | L391V.L409V.N413D. | SAGDMRAANLWPSPLMIKRSKKNSLALSLTAD |
| | mutant. | S463P.M517A.H524L | QMVSALLDAEPPILYSEYDPTRPFSEASMMGL |
| | N413D. | | LTNLADRELVHMINWAKRVPGFVDLTLHDQVH |
| | S463P. | | LLECAWLEILMIGVVWRSMEHPVKLLFAPNLV |
| | H524L. | | LDRDQGKCVEGMVEIFDMLLATSSRFRMMNLQ |
| | 83 | | GEEFVCLKSIILLNSGVYTFLPSTLKSLEEKD |
| | | | HIHRVLDKITDTLIHLMAKAGLTLQQQHQRLA |
| | | | QLLLILSHIRHASNKGMELLYSMKCKNVVPLY |
| | | | DLLLEAADAHRLHAPTSRGGASVEETDQSHLA |
| | | | TAGSTSSHSLQKYYITGEAEGFPATA (SEQ |
| | | | ID NO: 99) |
| SB09657 | ERT2. | N413D.S463P.L354I. | SAGDMRAANLWPSPLMIKRSKKNSLALSLTAD |
| | N413D. | L384M.H524L | QMVSALLDAEPPILYSEYDPTRPFSEASMMGL |
| | S463P. | | LTNLADREIVHMINWAKRVPGFVDLTLHDQVH |
| | H524L. | | LLECAWMEILMIGLVWRSMEHPVKLLFAPNLL |
| | mutant. | | LDRDQGKCVEGMVEIFDMLLATSSRFRMMNLQ |
| | 94 | | GEEFVCLKSIILLNSGVYTFLPSTLKSLEEKD |
| | | | HIHRVLDKITDTLIHLMAKAGLTLQQQHQRLA |
| | | | QLLLILSHIRHMSNKGMELLYSMKCKNVVPLY |
| | | | DLLLEAADAHRLHAPTSRGGASVEETDQSHLA |
| | | | TAGSTSSHSLQKYYITGEAEGFPATA (SEQ |
| | | | ID NO: 100) |
| SB09626 | ERT2. | N413D.S463P.M421L. | SAGDMRAANLWPSPLMIKRSKKNSLALSLTAD |
| | mutant. | L354I.H524L | QMVSALLDAEPPILYSEYDPTRPFSEASMMGL |
| | N413D. | | LTNLADREIVHMINWAKRVPGFVDLTLHDQVH |
| | S463P. | | LLECAWLEILMIGLVWRSMEHPVKLLFAPNLL |
| | H524L. | | LDRDQGKCVEGLVEIFDMLLATSSRFRMMNLQ |
| | 23 | | GEEFVCLKSIILLNSGVYTFLPSTLKSLEEKD |
| | | | HIHRVLDKITDTLIHLMAKAGLTLQQQHQRLA |
| | | | QLLLILSHIRHMSNKGMELLYSMKCKNVVPLY |
| | | | DLLLEAADAHRLHAPTSRGGASVEETDQSHLA |
| | | | TAGSTSSHSLQKYYITGEAEGFPATA (SEQ |
| | | | ID NO: 101) |
| SB03422 | WT ERT2 | | SEQ ID NO: 3 |

The next day, cells were split into no drug or drug titration conditions to evaluate performance at a range of endoxifen and estradiol concentrations. Performance of each ERT2 mutant transcriptional switch was evaluated by their ability to translocate and bind to the zinc finger binding site upstream of mCherry and initiate mCherry expression.

FIG. 26B shows log 10 fold activation across a range of endoxifen concentrations, normalized to no virus control. mCherry fold activation was calculated by first normalizing the gMFI mcherry values quantified by the background observed in the NV followed by dividing the normalized gMFI mCherry value in the drug treated condition by its corresponding ND sample. The background normalized ND (0) conditions were divided by the average NV gMFI mCherry value in order to reflect any basal activity seen by each mutant. FIG. 26C depicts induced mCherry expression plotted against basal activity for each of the tested ERT2 transcriptional switch constructs. As indicated in FIGS. 26B and 26C: SB09642, SB09657, SB09626, and SB09652 exhibited the highest fold activation and induced mCherry levels, as compared to WT (SB03422). Lead ERT2 mutants SB09642, SB09652, SB09657, SB09626 also displayed similar (or lower) basal activity to WT ERT2.

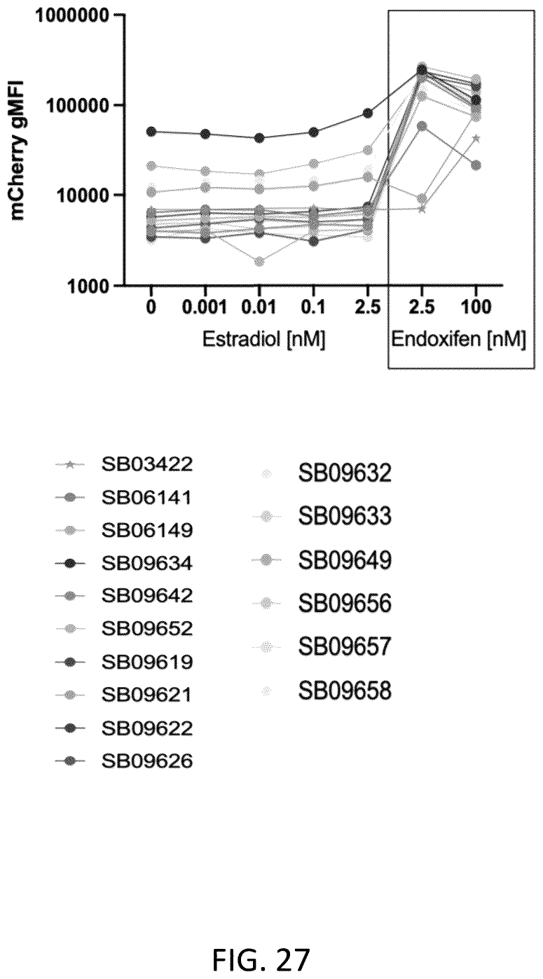

FIG. 27 shows mCherry gMFI after background subtraction across a range of estradiol and endoxifen concentrations. Results indicate that all lead ERT2 mutants maintained insensitivity to estradiol even at the ultra-high concentration of 2.5 nM, while maintaining robust sensitivity at 2.5 and 100 nM endoxifen.

### Example 11

### Evaluation of Exemplary ERT2 Mutant Transcriptional Switch Activity for Inducing Expression of an IL-12 Payload

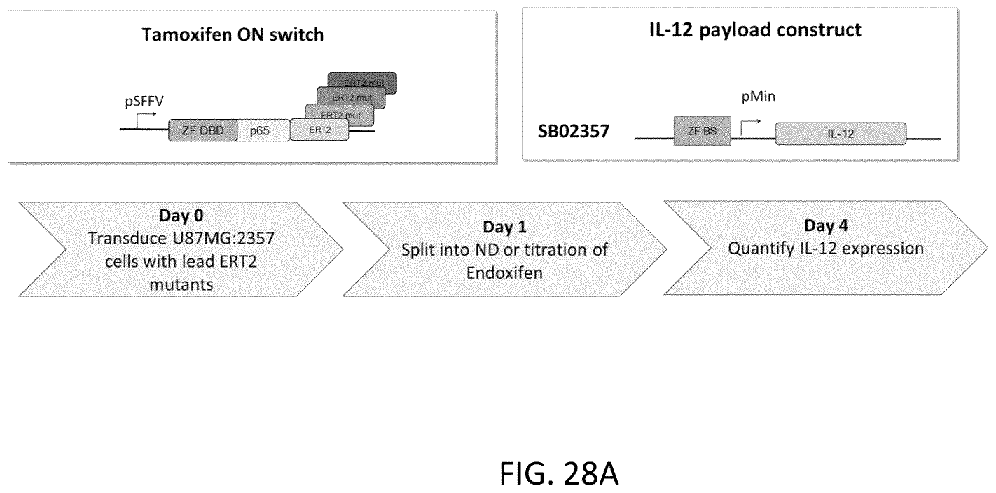

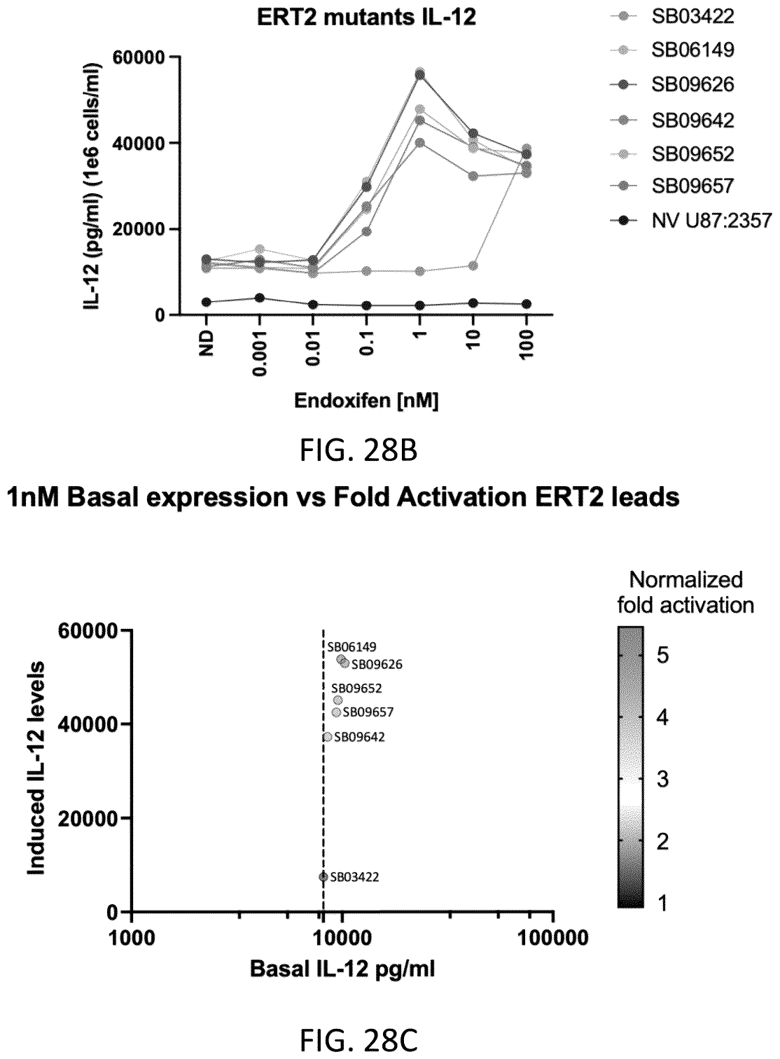

FIG. 28A depicts constructs and experimental methods for evaluating exemplary ERT2 mutant transcriptional switches for inducing expression of an IL-12 payload. U87MG cells were transduced with construct SB02357, comprising synthetic promoter comprising a 4×ZF-10 zinc finger binding site (SEQ ID NO: 59) linked to a minimal promoter operably linked to an IL-12 encoding sequence. This generated a U87MG: 2357 reporter cell line used for assessing the ability of ERT2 transcriptional switch constructs to activate transcription of the IL-12 payload in a ligand-dependent manner.

25k U87MG: 2357 IL-12 payload transduced line was transduced with MOI of 1 of the ERT2 constructs SB03422, SB06149, SB09626, SB09642, SB09652, and SB09657, which are described in Example 9. These were a subset of the ERT2 mutant transcriptional switch constructs that showed particular promise from the mCherry reporter experiment in Example 9. The next day, cells were split into ND or drug titration conditions to evaluate performance at a range of Endoxifen and concentrations. 72 hours later, supernatant was harvested for quantification of IL-12 by IL-12p70 ELISA. IL-12 levels were read with a spectrophotometer at wavelength 450 nm with wavelength correction to 540 nm according to manufacturer's protocol. A kit-provided standard to calculate detected levels of IL-12 in 100 μL of media harvested from ˜100k cells based on OD. Displayed values were scaled to reflect IL-12 levels as pg/mL produced by 1 million cells.

Results are shown in FIGS. 28B and 28C. FIG. 28B shows that the tested ERT2 mutant constructs SB06149, SB09626, SB09642, SB09652, and SB09657 induced IL-12 expression at concentrations as low as 0.1 nM endoxifen. FIG. 28C shows IL-12 levels of induced IL-12 levels plotted against basal IL-12 levels at 2.5 nM endoxifen. All tested mutants transcriptional switch constructs exhibited similar basal activity as the WT ERT2 transcriptional switch construct, and further exhibited much higher IL-12 induction at the 2.5 nM endoxifen concentration as compared to WT ERT2.

### Example 12

### Evaluation of Additional ERT2 Mutant Safety Switch Constructs

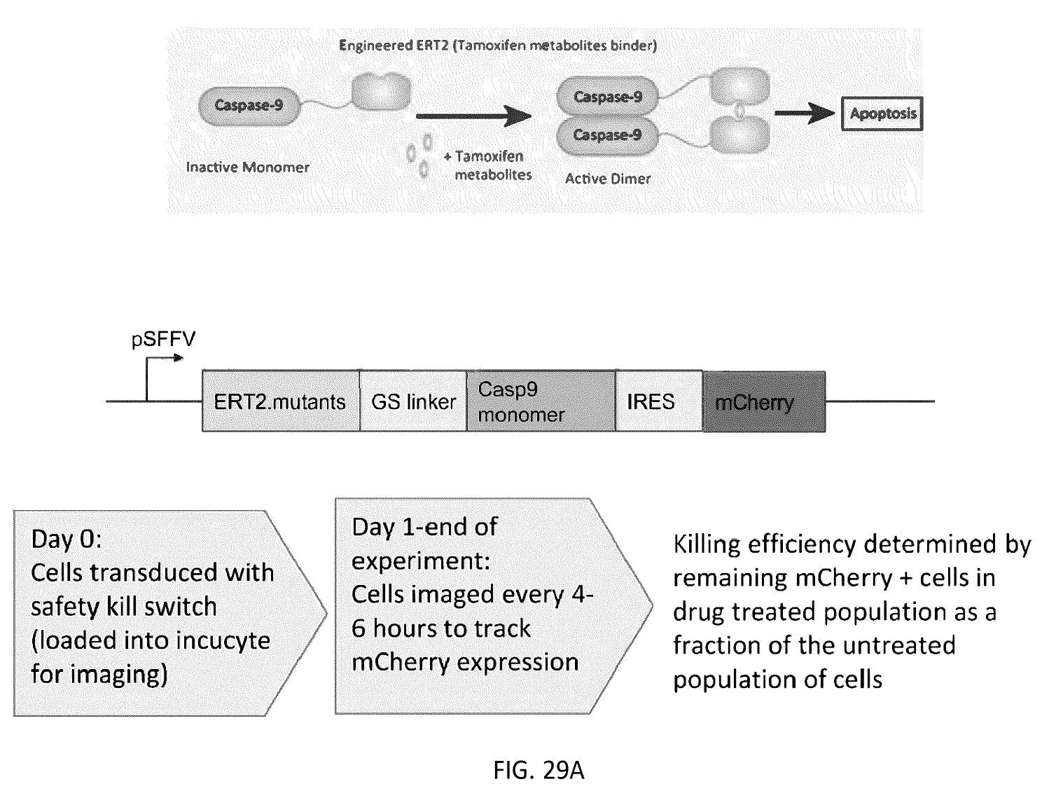

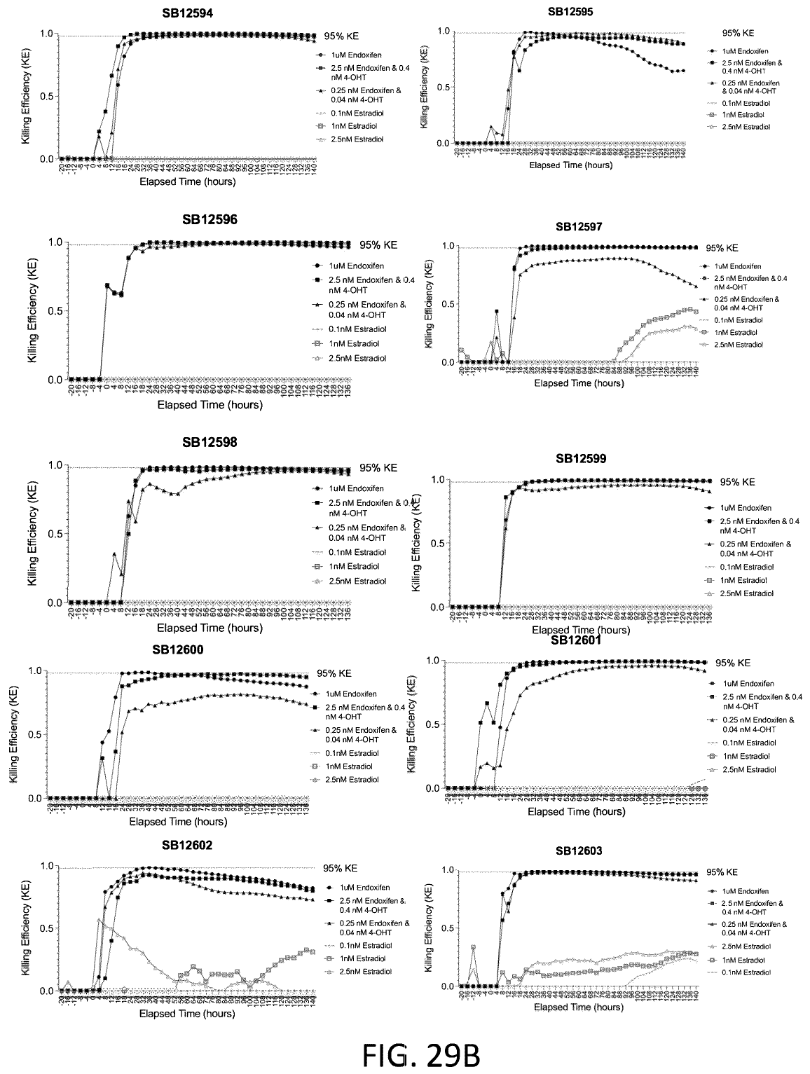

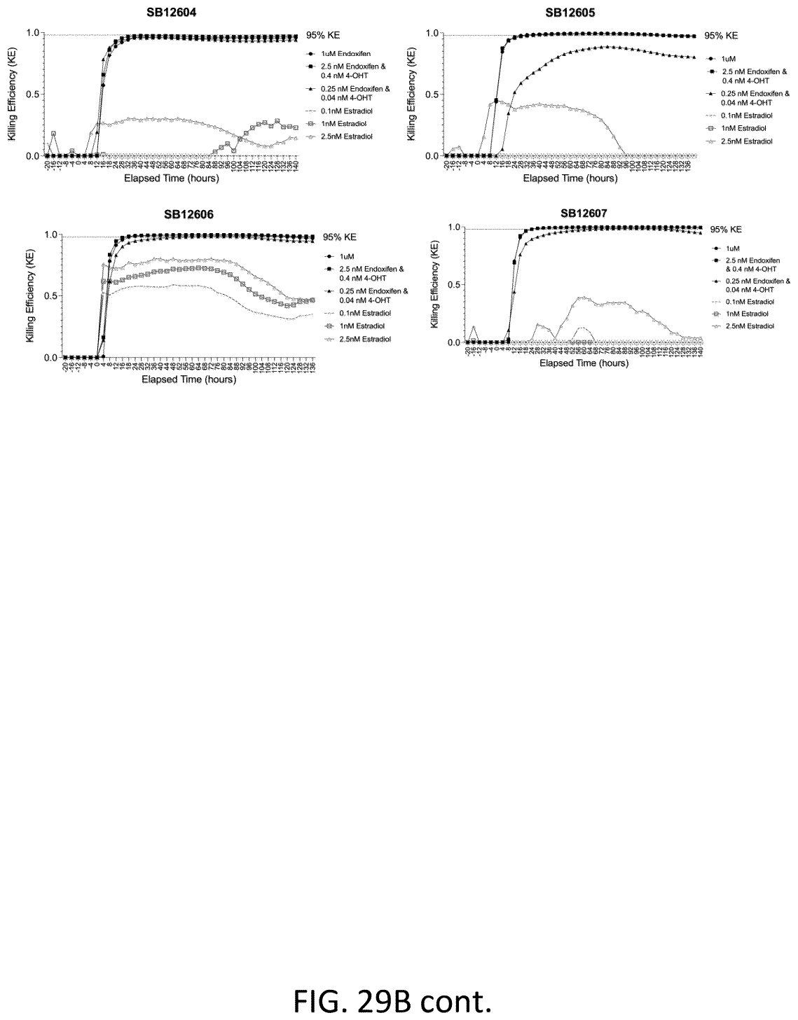

FIG. 29A shows an experimental workflow for evaluating suicide switch activity for a number of additional ERT2 mutant safety switch constructs.

A 24-well dish was seeded with 25,000 Lent-X HEK293T cells (Takara, Catalog #: 632180). The next day, the cells were transduced with 25,000 pg of virus for constructs SB12594-SB12607: ERT2mut-casp9 constructs tagged with RFP to track successfully transduced cells in a population. The constructs each take the form ERT2*mut-Casp9-IRES-RFP. See Table 24.

TABLE 24

| | | | ERT2 aa sequence (with |
|---|---|---|---|
| | | | starting Methionine, |
| | | | with optional A at the |
| | | | C-terminal end of the |
| SB | description | mutations to ERT2 | domain) |
| SB12594 | pSFFV: ERT2. | L391V.L409V.N413D.S463P. | MSAGDMRAANLWPSPLMIKRSKKNSLALS |
| | N413D.S463P. | M517A.M421L.L354I.H524L | LTADQMVSALLDAEPPILYSEYDPTRPFS |
| | H524L.mutant. | | EASMMGLLTNLADREIVHMINWAKRVPGF |
| | 41-GS linker | | VDLTLHDQVHLLECAWLEILMIGVVWRSM |
| | 1-iCasp9- | | EHPVKLLFAPNLVLDRDQGKCVEGLVEIF |
| | IRES-RFP | | DMLLATSSRFRMMNLQGEEFVCLKSIILL |
| | | | NSGVYTFLPSTLKSLEEKDHIHRVLDKIT |
| | | | DTLIHLMAKAGLTLQQQHORLAQLLLILS |
| | | | HIRHASNKGMELLYSMKCKNVVPLYDLLL |
| | | | EAADAHRLHAPTSRGGASVEETDQSHLAT |
| | | | AGSTSSHSLQKYYITGEAEGFPATA |
| | | | (SEQ ID NO: 105) |
| SB12595 | pSFFV: ERT2. | L391V.Q414E.N413D.S463P. | MSAGDMRAANLWPSPLMIKRSKKNSLALS |
| | N413D.S463P. | M421L.L354I.L384M.H524L | LTADQMVSALLDAEPPILYSEYDPTRPFS |
| | H524L.mutant. | | EASMMGLLTNLADREIVHMINWAKRVPGF |
| | 51-GS linker | | VDLTLHDQVHLLECAWMEILMIGVVWRSM |
| | 1-iCasp9- | | EHPVKLLFAPNLLLDRDEGKCVEGLVEIF |
| | IRES-RFP | | DMLLATSSRFRMMNLQGEEFVCLKSIILL |
| | | | NSGVYTFLPSTLKSLEEKDHIHRVLDKIT |
| | | | DTLIHLMAKAGLTLQQQHQRLAQLLLILS |
| | | | HIRHMSNKGMELLYSMKCKNVVPLYDLLL |
| | | | EAADAHRLHAPTSRGGASVEETDQSHLAT |
| | | | AGSTSSHSLQKYYITGEAEGFPATA |
| | | | (SEQ ID NO: 106) |
| SB12596 | pSFFV: ERT2. | L391V.L409V.N413D.S463P. | MSAGDMRAANLWPSPLMIKRSKKNSLALS |
| | N413D.S463P. | M517A.M421L.H524L | LTADQMVSALLDAEPPILYSEYDPTRPFS |
| | H524L.mutant. | | EASMMGLLTNLADRELVHMINWAKRVPGF |
| | 17-GS linker | | VDLTLHDQVHLLECAWLEILMIGVVWRSM |
| | 1-iCasp9- | | EHPVKLLFAPNLVLDRDQGKCVEGLVEIF |
| | IRES-RFP | | DMLLATSSRFRMMNLQGEEFVCLKSIILL |
| | | | NSGVYTFLPSTLKSLEEKDHIHRVLDKIT |
| | | | DTLIHLMAKAGLTLQQQHQRLAQLLLILS |
| | | | HIRHASNKGMELLYSMKCKNVVPLYDLLL |
| | | | EAADAHRLHAPTSRGGASVEETDQSHLAT |
| | | | AGSTSSHSLQKYYITGEAEGFPATA |
| | | | (SEQ ID NO: 158) |
| SB12597 | pSFFV: ERT2. | N413D.S463P.M421L.L354I. | MSAGDMRAANLWPSPLMIKRSKKNSLALS |
| | N413D.S463P. | H524L | LTADQMVSALLDAEPPILYSEYDPTRPFS |
| | H524L.mutant. | | EASMMGLLTNLADREIVHMINWAKRVPGF |
| | 23-GS linker | | VDLTLHDQVHLLECAWLEILMIGLVWRSM |
| | 1-iCasp9- | | EHPVKLLFAPNLLLDRDQGKCVEGLVEIF |
| | IRES-RFP | | DMLLATSSRFRMMNLQGEEFVCLKSIILL |
| | | | NSGVYTFLPSTLKSLEEKDHIHRVLDKIT |
| | | | DTLIHLMAKAGLTLQQQHQRLAQLLLILS |
| | | | HIRHMSNKGMELLYSMKCKNVVPLYDLLL |
| | | | EAADAHRLHAPTSRGGASVEETDQSHLAT |
| | | | AGSTSSHSLQKYYITGEAEGFPATA |
| | | | (SEQ ID NO: 159) |
| SB12598 | pSFFV: ERT2. | L391V.L409V.Q414E.N413D. | MSAGDMRAANLWPSPLMIKRSKKNSLALS |
| | N413D.S463P. | S463P.L354I.H524L | LTADQMVSALLDAEPPILYSEYDPTRPFS |
| | H524L.mutant. | | EASMMGLLTNLADREIVHMINWAKRVPGF |
| | 36-GS linker | | VDLTLHDQVHLLECAWLEILMIGVVWRSM |
| | 1-iCasp9- | | EHPVKLLFAPNLVLDRDEGKCVEGMVEIF |
| | IRES-RFP | | DMLLATSSRFRMMNLQGEEFVCLKSIILL |
| | | | NSGVYTFLPSTLKSLEEKDHIHRVLDKIT |
| | | | DTLIHLMAKAGLTLQQQHORLAQLLLILS |
| | | | HIRHMSNKGMELLYSMKCKNVVPLYDLLL |
| | | | EAADAHRLHAPTSRGGASVEETDQSHLAT |
| | | | AGSTSSHSLQKYYITGEAEGFPATA |
| | | | (SEQ ID NO: 160) |
| SB12599 | pSFFV: ERT2. | L391V.L409V.N413D.S463P. | MSAGDMRAANLWPSPLMIKRSKKNSLALS |
| | N413D.S463P. | M421L.L354I.L384M.H524L | LTADQMVSALLDAEPPILYSEYDPTRPFS |
| | H524L.mutant. | | EASMMGLLTNLADREIVHMINWAKRVPGF |
| | 66-GS linker | | VDLTLHDQVHLLECAWMEILMIGVVWRSM |
| | 1-iCasp9- | | EHPVKLLFAPNLVLDRDQGKCVEGLVEIF |
| | IRES-RFP | | DMLLATSSRFRMMNLQGEEFVCLKSIILL |
| | | | NSGVYTFLPSTLKSLEEKDHIHRVLDKIT |
| | | | DTLIHLMAKAGLTLQQQHQRLAQLLLILS |
| | | | HIRHMSNKGMELLYSMKCKNVVPLYDLLL |
| | | | EAADAHRLHAPTSRGGASVEETDQSHLAT |
| | | | AGSTSSHSLQKYYITGEAEGFPATA |
| | | | (SEQ ID NO: 161) |
| SB12600 | pSFFV: ERT2. | L391V.L409V.Q414E.N413D. | MSAGDMRAANLWPSPLMIKRSKKNSLALS |
| | N413D.S463P. | S463P.M517A.H524L | LTADQMVSALLDAEPPILYSEYDPTRPFS |
| | H524L.mutant. | | EASMMGLLTNLADRELVHMINWAKRVPGF |
| | 25-GS linker | | VDLTLHDQVHLLECAWLEILMIGVVWRSM |
| | 1-iCasp9- | | EHPVKLLFAPNLVLDRDEGKCVEGMVEIF |
| | IRES-RFP | | DMLLATSSRFRMMNLQGEEFVCLKSIILL |
| | | | NSGVYTFLPSTLKSLEEKDHIHRVLDKIT |
| | | | DTLIHLMAKAGLTLQQQHQRLAQLLLILS |
| | | | HIRHASNKGMELLYSMKCKNVVPLYDLLL |
| | | | EAADAHRLHAPTSRGGASVEETDQSHLAT |
| | | | AGSTSSHSLQKYYITGEAEGFPATA |
| | | | (SEQ ID NO: 103) |
| SB12601 | pSFFV: ERT2. | L409V.N413D.S463P.M421L. | MSAGDMRAANLWPSPLMIKRSKKNSLALS |
| | N413D.S463P. | L384M.H524L | LTADQMVSALLDAEPPILYSEYDPTRPFS |
| | H524L.mutant. | | EASMMGLLTNLADRELVHMINWAKRVPGF |
| | 14-GS linker | | VDLTLHDQVHLLECAWMEILMIGLVWRSM |
| | 1-iCasp9- | | EHPVKLLFAPNLVLDRDQGKCVEGLVEIF |
| | IRES-RFP | | DMLLATSSRFRMMNLQGEEFVCLKSIILL |
| | | | NSGVYTFLPSTLKSLEEKDHIHRVLDKIT |
| | | | DTLIHLMAKAGLTLQQQHQRLAQLLLILS |
| | | | HIRHMSNKGMELLYSMKCKNVVPLYDLLL |
| | | | EAADAHRLHAPTSRGGASVEETDQSHLAT |
| | | | AGSTSSHSLQKYYITGEAEGFPATA |
| | | | (SEQ ID NO: 104) |
| SB12602 | pSFFV: ERT2. | L391V.Q414E.N413D.S463P. | MSAGDMRAANLWPSPLMIKRSKKNSLALS |
| | N413D.S463P. | M517A.M421L.L354I.H524L | LTADQMVSALLDAEPPILYSEYDPTRPFS |
| | H524L.mutant. | | EASMMGLLTNLADREIVHMINWAKRVPGF |
| | 97-GS linker | | VDLTLHDQVHLLECAWLEILMIGVVWRSM |
| | 1-iCasp9- | | EHPVKLLFAPNLLLDRDEGKCVEGLVEIF |
| | IRES-RFP | | DMLLATSSRFRMMNLQGEEFVCLKSIILL |
| | | | NSGVYTFLPSTLKSLEEKDHIHRVLDKIT |
| | | | DTLIHLMAKAGLTLQQQHORLAQLLLILS |
| | | | HIRHASNKGMELLYSMKCKNVVPLYDLLL |
| | | | EAADAHRLHAPTSRGGASVEETDQSHLAT |
| | | | AGSTSSHSLQKYYITGEAEGFPATA |
| | | | (SEQ ID NO: 162) |
| SB12603 | pSFFV: ERT2. | L391V.N413D.S463P.M517A. | MSAGDMRAANLWPSPLMIKRSKKNSLALS |
| | N413D.S463P. | M421L.L354I.H524L | LTADQMVSALLDAEPPILYSEYDPTRPFS |
| | H524L.mutant. | | EASMMGLLTNLADREIVHMINWAKRVPGF |
| | 37-GS linker | | VDLTLHDQVHLLECAWLEILMIGVVWRSM |
| | 1-iCasp9- | | EHPVKLLFAPNLLLDRDQGKCVEGLVEIF |
| | IRES-RFP | | DMLLATSSRFRMMNLQGEEFVCLKSIILL |
| | | | NSGVYTFLPSTLKSLEEKDHIHRVLDKIT |
| | | | DTLIHLMAKAGLTLQQQHQRLAQLLLILS |
| | | | HIRHASNKGMELLYSMKCKNVVPLYDLLL |
| | | | EAADAHRLHAPTSRGGASVEETDQSHLAT |
| | | | AGSTSSHSLQKYYITGEAEGFPATA |
| | | | (SEQ ID NO: 163) |
| SB12604 | pSFFV: ERT2. | L391V.N413D.S463P.M517A. | MSAGDMRAANLWPSPLMIKRSKKNSLALS |
| | N413D.S463P. | L384M.H524L | LTADQMVSALLDAEPPILYSEYDPTRPFS |
| | H524L.mutant. | | EASMMGLLTNLADRELVHMINWAKRVPGF |
| | 128-GS linker | | VDLTLHDQVHLLECAWMEILMIGVVWRSM |
| | 1-iCasp9- | | EHPVKLLFAPNLLLDRDQGKCVEGMVEIF |
| | IRES-RFP | | DMLLATSSRFRMMNLQGEEFVCLKSIILL |
| | | | NSGVYTFLPSTLKSLEEKDHIHRVLDKIT |
| | | | DTLIHLMAKAGLTLQQQHQRLAQLLLILS |
| | | | HIRHASNKGMELLYSMKCKNVVPLYDLLL |
| | | | EAADAHRLHAPTSRGGASVEETDQSHLAT |
| | | | AGSTSSHSLQKYYITGEAEGFPATA |
| | | | (SEQ ID NO: 164) |
| SB12605 | pSFFV: ERT2. | N413D.S463P.L354I.L384M. | MSAGDMRAANLWPSPLMIKRSKKNSLALS |
| | N413D.S463P. | H524L | LTADQMVSALLDAEPPILYSEYDPTRPFS |
| | H524L.mutant. | | EASMMGLLTNLADREIVHMINWAKRVPGF |
| | 94-GS linker | | VDLTLHDQVHLLECAWMEILMIGLVWRSM |
| | 1-iCasp9- | | EHPVKLLFAPNLLLDRDQGKCVEGMVEIF |
| | IRES-RFP | | DMLLATSSRFRMMNLQGEEFVCLKSIILL |
| | | | NSGVYTFLPSTLKSLEEKDHIHRVLDKIT |
| | | | DTLIHLMAKAGLTLQQQHQRLAQLLLILS |
| | | | HIRHMSNKGMELLYSMKCKNVVPLYDLLL |
| | | | EAADAHRLHAPTSRGGASVEETDQSHLAT |
| | | | AGSTSSHSLQKYYITGEAEGFPATA |
| | | | (SEQ ID NO: 165) |
| SB12606 | pSFFV: ERT2. | L391V.L409V.N413D.S463P. | MSAGDMRAANLWPSPLMIKRSKKNSLALS |
| | N413D.S463P. | M517A.H524L | LTADQMVSALLDAEPPILYSEYDPTRPFS |
| | H524L.mutant. | | EASMMGLLTNLADRELVHMINWAKRVPGF |
| | 83-GS linker | | VDLTLHDQVHLLECAWLEILMIGVVWRSM |
| | 1-iCasp9- | | EHPVKLLFAPNLVLDRDQGKCVEGMVEIF |
| | IRES-RFP | | DMLLATSSRFRMMNLQGEEFVCLKSIILL |
| | | | NSGVYTFLPSTLKSLEEKDHIHRVLDKIT |
| | | | DTLIHLMAKAGLTLQQQHQRLAQLLLILS |
| | | | HIRHASNKGMELLYSMKCKNVVPLYDLLL |
| | | | EAADAHRLHAPTSRGGASVEETDQSHLAT |
| | | | AGSTSSHSLQKYYITGEAEGFPATA |
| | | | (SEQ ID NO: 166) |
| SB12607 | pSFFV: ERT2. | L384M.L391V.N413D.M421L. | MSAGDMRAANLWPSPLMIKRSKKNSLALS |
| | mut81 (with | S463P.H524L | LTADQMVSALLDAEPPILYSEYDPTRPFS |
| | S and A)-GS | | EASMMGLLTNLADRELVHMINWAKRVPGF |
| | linker1- | | VDLTLHDQVHLLECAWMEILMIGVVWRSM |
| | iCasp9-IRES- | | EHPVKLLFAPNLLLDRDQGKCVEGLVEIF |
| | RFP | | DMLLATSSRFRMMNLQGEEFVCLKSIILL |
| | | | NSGVYTFLPSTLKSLEEKDHIHRVLDKIT |
| | | | DTLIHLMAKAGLTLQQQHQRLAQLLLILS |
| | | | HIRHMSNKGMELLYSMKCKNVVPLYDLLL |
| | | | EAADAHRLHAPTSRGGASVEETDQSHLAT |
| | | | AGSTSSHSLQKYYITGEAEGFPATA |
| | | | (SEQ ID NO: 167) |

The next day, transduced cells were split into a 96-well plate with ˜3,000 cells in each well and loaded into an incucyte to quantify cell expansion via images every 2 hours detecting mCherry expression. Media was exchanged 24 hours later with new media with indicated concentration of 4-OHT or no drug added. Cells continued to be imaged by the incucyte every 4-8 hours for 72-140 hours post exchange of media.

>95% killing efficiency is considered complete killing in the assay by treatment of mCherry expressing cells with 2 uM Puromycin which is known to kill HEK293T cells within 48 hours of application. 2.5 nM Endoxifen and 0.4 nM 4-OHT reflect the estimated concentrations of tamoxifen metabolites in the brain, tissue, organs, and solid tumors. 0.25 nM endoxifen and 0.04 nM 4-OHT reflect the estimated concentration of tamoxifen metabolites in the serum. Both estimates are based on clinical data measuring tamoxifen metabolite concentrations in the serum of people taking the FDA-approved dose of tamoxifen orally (20 mg/day). It has been observed in vivo that tamoxifen 10× concentrates in tissues, organs, solid tumor, brain.

Results are shown in FIG. 29B. ERT2 mutants in constructs SB12594, SB12596, achieved complete killing within 48 hours of induction with brain (solid square) and serum (solid triangle) concentrations of tamoxifen metabolites. SB12598, SB12599, SB12600, and SB12601 switches achieved complete killing at brain concentration of tamoxifen metabolites, but not serum concentrations. SB12595 achieved and maintained ˜90% killing efficiency within 48 hours at both brain and serum concentrations of tamoxifen metabolites. Furthermore, the engineered ERT2 mutants in constructs SB12594-12596 and SB12598-12601 remained insensitive to physiological concentrations of estradiol.

TABLE 18
Sequences

| | SEQ | |
|---|---|---|
| | ID | |
| Name | NO | Sequence |
| Estrogen | 1 | MTMTLHTKASGMALLHQIQGNELEPLNRPQLKIPLERPLGEVYL |
| Receptor | | DSSKPAVYNYPEGAAYEFNAAAAANAQVYGQTGLPYGPGSEAA |
| (Amino Acid | | AFGSNGLGGFPPLNSVSPSPLMLLHPPPQLSPFLQPHGQQVPYYL |
| Sequence) | | ENEPSGYTVREAGPPAFYRPNSDNRRQGGRERLASTNDKGSMA |
| Italics = amino | | MESAKETRYCAVCNDYASGYHYGVWSCEGCKAFFKRSIQGHND |
| acid positions | | YMCPATNQCTIDKNRRKSCQACRLRKCYEVGMMKGGIRKDRRG |
| 282-595 | | GRMLKHKRQRDDGEGRGEVGSAGDMRAANLWPSPLMIKRSKK |
| | | NSLALSLTADQMVSALLDAEPPILYSEYDPTRPFSEASMMGLLTN |
| | | LADRELVHMINWAKRVPGFVDLTLHDQVHLLECAWLEILMIGL |
| | | VWRSMEHPGKLLFAPNLLLDRNQGKCVEGMVEIFDMLLATSSR |
| | | FRMMNLQGEEFVCLKSIILLNSGVYTFLSSTLKSLEEKDHIHRVL |
| | | DKITDTLIHLMAKAGLTLQQQHQRLAQLLLILSHIRHMSNKGME |
| | | HLYSMKCKNVVPLYDLLLEMLDAHRLHAPTSRGGASVEETDQS |
| | | HLATAGSTSSHSLQKYYITGEAEGFPATV |
| Example ERT2 | 2 | SAGDMRAANLWPSPLMIKRSKKNSLALSLTADQMVSALLDAEPPILY |
| (positions 282- | | SEYDPTRPFSEASMMGLLTNLADRELVHMINWAKRVPGFVDLTLHD |
| 594 of SEQ ID | | QVHLLECAWLEILMIGLVWRSMEHPVKLLFAPNLLLDRNQGKCVEG |
| NO: 1) | | MVEIFDMLLATSSRFRMMNLQGEEFVCLKSIILLNSGVYTFLSSTLKSL |
| | | EEKDHIHRVLDKITDTLIHLMAKAGLTLQQQHQRLAQLLLILSHIRHM |
| | | SNKGMEHLYSMKCKNVVPLYDLLLEAADAHRLHAPTSRGGASVEET |
| | | DQSHLATAGSTSSHSLQKYYITGEAEGFPAT |
| Example ERT2 | 3 | SAGDMRAANLWPSPLMIKRSKKNSLALSLTADQMVSALLDAEPPILY |
| (positions 282- | | SEYDPTRPFSEASMMGLLTNLADRELVHMINWAKRVPGFVDLTLHD |
| 595 of SEQ ID | | QVHLLECAWLEILMIGLVWRSMEHPVKLLFAPNLLLDRNQGKCVEG |
| NO: 1) | | MVEIFDMLLATSSRFRMMNLQGEEFVCLKSIILLNSGVYTFLSSTLKSL |
| | | EEKDHIHRVLDKITDTLIHLMAKAGLTLQQQHQRLAQLLLILSHIRHM |
| | | SNKGMEHLYSMKCKNVVPLYDLLLEAADAHRLHAPTSRGGASVEET |
| | | DQSHLATAGSTSSHSLQKYYITGEAEGFPATA |
| Example ERT2 | 42 | AGDMRAANLWPSPLMIKRSKKNSLALSLTADQMVSALLDAEPPILYS |
| (positions 283- | | EYDPTRPFSEASMMGLLTNLADRELVHMINWAKRVPGFVDLTLHDQ |
| 594 of SEQ ID | | VHLLECAWLEILMIGLVWRSMEHPVKLLFAPNLLLDRNQGKCVEGM |
| NO: 1) | | VEIFDMLLATSSRFRMMNLQGEEFVCLKSIILLNSGVYTFLSSTLKSLE |
| | | EKDHIHRVLDKITDTLIHLMAKAGLTLQQQHQRLAQLLLILSHIRHMS |
| | | NKGMEHLYSMKCKNVVPLYDLLLEAADAHRLHAPTSRGGASVEETD |
| | | QSHLATAGSTSSHSLQKYYITGEAEGFPAT |
| Example ERT2 | 168 | AGDMRAANLWPSPLMIKRSKKNSLALSLTADQMVSALLDAEPPILYS |
| (positions 283- | | EYDPTRPFSEASMMGLLTNLADRELVHMINWAKRVPGFVDLTLHDQ |
| 595 of SEQ ID | | VHLLECAWLEILMIGLVWRSMEHPVKLLFAPNLLLDRNQGKCVEGM |
| NO: 1) | | VEIFDMLLATSSRFRMMNLQGEEFVCLKSIILLNSGVYTFLSSTLKSLE |
| | | EKDHIHRVLDKITDTLIHLMAKAGLTLQQQHQRLAQLLLILSHIRHMS |
| | | NKGMEHLYSMKCKNVVPLYDLLLEAADAHRLHAPTSRGGASVEETD |
| | | QSHLATAGSTSSHSLQKYYITGEAEGFPATA |
| Peptide Linker | 4 | GGGGSGGGGSGGGGSVDGF |
| Peptide Linker | 5 | ASGGGGSAS |
| Zinc Finger | 6 | SRPGERPFQCRICMRNFSRRHGLDRHTRTHTGEKPFQCRICMRNFSDH |
| Protein Domain | | SSLKRHLRTHTGSQKPFQCRICMRNFSVRHNLTRHLRTHTGEKPFQCR |
| ZF10-1 | | ICMRNFSDHSNLSRHLKTHTGSQKPFQCRICMRNFSQRSSLVRHLRTH |
| | | TGEKPFQCRICMRNFSESGHLKRHLRTHLRGS |
| minimal | 7 | AGAGGGTATATAATGGAAGCTCGACTTCCAG |
| promoter; minP | | |
| NFkB response | 8 | GGGAATTTCCGGGGACTTTCCGGGAATTTCCGGGGACTTTCCGGGA |
| element protein | | ATTTCC |
| promoter; 5x | | |
| NFkB-RE | | |
| CREB response | 9 | CACCAGACAGTGACGTCAGCTGCCAGATCCCATGGCCGTCATACT |
| element protein | | GTGACGTCTTTCAGACACCCCATTGACGTCAATGGGAGAA |
| promoter; 4x | | |
| CRE | | |
| NFAT response | 10 | GGAGGAAAAACTGTTTCATACAGAAGGCGTGGAGGAAAAACTGTT |
| element protein | | TCATACAGAAGGCGTGGAGGAAAAACTGTTTCATACAGAAGGCGT |
| promoter; 3x | | |
| NFAT binding | | |
| sites | | |
| SRF response | 11 | AGGATGTCCATATTAGGACATCTAGGATGTCCATATTAGGACATCT |
| element protein | | AGGATGTCCATATTAGGACATCTAGGATGTCCATATTAGGACATCT |
| promoter; 5x | | AGGATGTCCATATTAGGACATCT |
| SRE | | |
| SRF response | 12 | AGTATGTCCATATTAGGACATCTACCATGTCCATATTAGGACATCT |
| element protein | | ACTATGTCCATATTAGGACATCTTGTATGTCCATATTAGGACATCT |
| promoter 2; 5x | | AAAATGTCCATATTAGGACATCT |
| SRF-RE | | |
| AP1 response | 13 | TGAGTCAGTGACTCAGTGAGTCAGTGACTCAGTGAGTCAGTGACT |
| element protein | | CAG |
| promoter; 6x | | |
| AP1-RE | | |
| TCF-LEF | 14 | AGATCAAAGGGTTTAAGATCAAAGGGCTTAAGATCAAAGGGTATA |
| response | | AGATCAAAGGGCCTAAGATCAAAGGGACTAAGATCAAAGGGTTTA |
| element | | AGATCAAAGGGCTTAAGATCAAAGGGCCTA |
| promoter; 8x | | |
| TCF-LEF-RE | | |
| SBEx4 | 15 | GTCTAGACGTCTAGACGTCTAGACGTCTAGAC |
| SMAD2/3- | 16 | CAGACACAGACACAGACACAGACA |
| CAGACA x4 | | |
| STAT3 binding | 17 | GGATCCGGTACTCGAGATCTGCGATCTAAGTAAGCTTGGCATTCCG |
| site | | GTACTGTTGGTAAAGCCAC |
| CMV | 18 | GTTGACATTGATTATTGACTAGTTATTAATAGTAATCAATTACGGG |
| | | GTCATTAGTTCATAGCCCATATATGGAGTTCCGCGTTACATAACTT |
| | | ACGGTAAATGGCCCGCCTGGCTGACCGCCCAACGACCCCCGCCCA |
| | | TTGACGTCAATAATGACGTATGTTCCCATAGTAACGCCAATAGGG |
| | | ACTTTCCATTGACGTCAATGGGTGGAGTATTTACGGTAAACTGCCC |
| | | ACTTGGCAGTACATCAAGTGTATCATATGCCAAGTACGCCCCCTAT |
| | | TGACGTCAATGACGGTAAATGGCCCGCCTGGCATTATGCCCAGTA |
| | | CATGACCTTATGGGACTTTCCTACTTGGCAGTACATCTACGTATTA |
| | | GTCATCGCTATTACCATGGTGATGCGGTTTTGGCAGTACATCAATG |
| | | GGCGTGGATAGCGGTTTGACTCACGGGGATTTCCAAGTCTCCACCC |
| | | CATTGACGTCAATGGGAGTTTGTTTTGGCACCAAAATCAACGGGA |
| | | CTTTCCAAAATGTCGTAACAACTCCGCCCCATTGACGCAAATGGGC |
| | | GGTAGGCGTGTACGGTGGGAGGTCTATATAAGCAGAGCTC |
| EF1a | 19 | GGCTCCGGTGCCCGTCAGTGGGCAGAGCGCACATCGCCCACAGTC |
| | | CCCGAGAAGTTGGGGGGAGGGGTCGGCAATTGAACCGGTGCCTAG |
| | | AGAAGGTGGCGCGGGGTAAACTGGGAAAGTGATGCCGTGTACTGG |
| | | CTCCGCCTTTTTCCCGAGGGTGGGGGAGAACCGTATATAAGTGCA |
| | | GTAGTCGCCGTGAACGTTCTTTTTCGCAACGGGTTTGCCGCCAGAA |
| | | CACAGGTAAGTGCCGTGTGTGGTTCCCGCGGGCCTGGCCTCTTTAC |
| | | GGGTTATGGCCCTTGCGTGCCTTGAATTACTTCCACCTGGCTGCAG |
| | | TACGTGATTCTTGATCCCGAGCTTCGGGTTGGAAGTGGGTGGGAG |
| | | AGTTCGAGGCCTTGCGCTTAAGGAGCCCCTTCGCCTCGTGCTTGAG |
| | | TTGAGGCCTGGCCTGGGCGCTGGGGCCGCCGCGTGCGAATCTGGT |
| | | GGCACCTTCGCGCCTGTCTCGCTGCTTTCGATAAGTCTCTAGCCAT |
| | | TTAAAATTTTTGATGACCTGCTGCGACGCTTTTTTTCTGGCAAGAT |
| | | AGTCTTGTAAATGCGGGCCAAGATCTGCACACTGGTATTTCGGTTT |
| | | TTGGGGCCGCGGGCGGCGACGGGGCCCGTGCGTCCCAGCGCACAT |
| | | GTTCGGCGAGGCGGGGCCTGCGAGCGCGACCACCGAGAATCGGAC |
| | | GGGGGTAGTCTCAAGCTGGCCGGCCTGCTCTGGTGCCTGTCCTCGC |
| | | GCCGCCGTGTATCGCCCCGCCCCGGGCGGCAAGGCTGGCCCGGTC |
| | | GGCACCAGTTGCGTGAGCGGAAAGATGGCCGCTTCCCGGTCCTGC |
| | | TGCAGGGAGCTCAAAATGGAGGACGCGGCGCTCGGGAGAGCGGG |
| | | CGGGTGAGTCACCCACACAAAGGAAAAGGGCCTTTCCGTCCTCAG |
| | | CCGTCGCTTCATGTGACTCCACGGAGTACCGGGCGCCGTCCAGGC |
| | | ACCTCGATTAGTTCTCGAGCTTTTGGAGTACGTCGTCTTTAGGTTG |
| | | GGGGGAGGGGTTTTATGCGATGGAGTTTCCCCACACTGAGTGGGT |
| | | GGAGACTGAAGTTAGGCCAGCTTGGCACTTGATGTAATTCTCCTTG |
| | | GAATTTGCCCTTTTTGAGTTTGGATCTTGGTTCATTCTCAAGCCTCA |
| | | GACAGTGGTTCAAAGTTTTTTTCTTCCATTTCAGGTGTCGTGA |
| EFS | 20 | GGATCTGCGATCGCTCCGGTGCCCGTCAGTGGGCAGAGCGCACAT |
| | | CGCCCACAGTCCCCGAGAAGTTGGGGGGAGGGGTCGGCAATTGAA |
| | | CCGGTGCCTAGAGAAGGTGGCGCGGGGTAAACTGGGAAAGTGATG |
| | | TCGTGTACTGGCTCCGCCTTTTTCCCGAGGGTGGGGGAGAACCGTA |
| | | TATAAGTGCAGTAGTCGCCGTGAACGTTCTTTTTCGCAACGGGTTT |
| | | GCCGCCAGAACACAGCTGAAGCTTCGAGGGGCTCGCATCTCTCCTT |
| | | CACGCGCCCGCCGCCCTACCTGAGGCCGCCATCCACGCCGGTTGA |
| | | GTCGCGTTCTGCCGCCTCCCGCCTGTGGTGCCTCCTGAACTGCGTC |
| | | CGCCGTCTAGGTAAGTTTAAAGCTCAGGTCGAGACCGGGCCTTTGT |
| | | CCGGCGCTCCCTTGGAGCCTACCTAGACTCAGCCGGCTCTCCACGC |
| | | TTTGCCTGACCCTGCTTGCTCAACTCTACGTCTTTGTTTCGTTTTCT |
| | | GTTCTGCGCCGTTACAGATCCAAGCTGTGACCGGCGCCTAC |
| MND | 21 | TTTATTTAGTCTCCAGAAAAAGGGGGGAATGAAAGACCCCACCTG |
| | | TAGGTTTGGCAAGCTAGGATCAAGGTTAGGAACAGAGAGACAGCA |
| | | GAATATGGGCCAAACAGGATATCTGTGGTAAGCAGTTCCTGCCCC |
| | | GGCTCAGGGCCAAGAACAGTTGGAACAGCAGAATATGGGCCAAA |
| | | CAGGATATCTGTGGTAAGCAGTTCCTGCCCCGGCTCAGGGCCAAG |
| | | AACAGATGGTCCCCAGATGCGGTCCCGCCCTCAGCAGTTTCTAGA |
| | | GAACCATCAGATGTTTCCAGGGTGCCCCAAGGACCTGAAATGACC |
| | | CTGTGCCTTATTTGAACTAACCAATCAGTTCGCTTCTCGCTTCTGTT |
| | | CGCGCGCTTCTGCTCCCCGAGCTCAATAAAAGAGCCCA |
| PGK | 22 | GGGGTTGGGGTTGCGCCTTTTCCAAGGCAGCCCTGGGTTTGCGCAG |
| | | GGACGCGGCTGCTCTGGGCGTGGTTCCGGGAAACGCAGCGGCGCC |
| | | GACCCTGGGTCTCGCACATTCTTCACGTCCGTTCGCAGCGTCACCC |
| | | GGATCTTCGCCGCTACCCTTGTGGGCCCCCCGGCGACGCTTCCTGC |
| | | TCCGCCCCTAAGTCGGGAAGGTTCCTTGCGGTTCGCGGCGTGCCGG |
| | | ACGTGACAAACGGAAGCCGCACGTCTCACTAGTACCCTCGCAGAC |
| | | GGACAGCGCCAGGGAGCAATGGCAGCGCGCCGACCGCGATGGGC |
| | | TGTGGCCAATAGCGGCTGCTCAGCGGGGCGCGCCGAGAGCAGCGG |
| | | CCGGGAAGGGGCGGTGCGGGAGGCGGGGTGTGGGGCGGTAGTGT |
| | | GGGCCCTGTTCCTGCCCGCGCGGTGTTCCGCATTCTGCAAGCCTCC |
| | | GGAGCGCACGTCGGCAGTCGGCTCCCTCGTTGACCGAATCACCGA |
| | | CCTCTCTCCCCAG |
| SFFV | 23 | GTAACGCCATTTTGCAAGGCATGGAAAAATACCAAACCAAGAATA |
| | | GAGAAGTTCAGATCAAGGGCGGGTACATGAAAATAGCTAACGTTG |
| | | GGCCAAACAGGATATCTGCGGTGAGCAGTTTCGGCCCCGGCCCGG |
| | | GGCCAAGAACAGATGGTCACCGCAGTTTCGGCCCCGGCCCGAGGC |
| | | CAAGAACAGATGGTCCCCAGATATGGCCCAACCCTCAGCAGTTTC |
| | | TTAAGACCCATCAGATGTTTCCAGGCTCCCCCAAGGACCTGAAATG |
| | | ACCCTGCGCCTTATTTGAATTAACCAATCAGCCTGCTTCTCGCTTCT |
| | | GTTCGCGCGCTTCTGCTTCCCGAGCTCTATAAAAGAGCTCACAACC |
| | | CCTCACTCGGCGCGCCAGTCCTCCGACAGACTGAGTCGCCCGGG |
| SV40 | 24 | CTGTGGAATGTGTGTCAGTTAGGGTGTGGAAAGTCCCCAGGCTCCC |
| | | CAGCAGGCAGAAGTATGCAAAGCATGCATCTCAATTAGTCAGCAA |
| | | CCAGGTGTGGAAAGTCCCCAGGCTCCCCAGCAGGCAGAAGTATGC |
| | | AAAGCATGCATCTCAATTAGTCAGCAACCATAGTCCCGCCCCTAAC |
| | | TCCGCCCATCCCGCCCCTAACTCCGCCCAGTTCCGCCCATTCTCCG |
| | | CCCCATGGCTGACTAATTTTTTTTATTTATGCAGAGGCCGAGGCCG |
| | | CCTCTGCCTCTGAGCTATTCCAGAAGTAGTGAGGAGGCTTTTTTGG |
| | | AGGCCTAGGCTTTTGCAAAAAGCT |
| UbC | 25 | GCGCCGGGTTTTGGCGCCTCCCGCGGGCGCCCCCCTCCTCACGGCG |
| | | AGCGCTGCCACGTCAGACGAAGGGCGCAGGAGCGTTCCTGATCCT |
| | | TCCGCCCGGACGCTCAGGACAGCGGCCCGCTGCTCATAAGACTCG |
| | | GCCTTAGAACCCCAGTATCAGCAGAAGGACATTTTAGGACGGGAC |
| | | TTGGGTGACTCTAGGGCACTGGTTTTCTTTCCAGAGAGCGGAACAG |
| | | GCGAGGAAAAGTAGTCCCTTCTCGGCGATTCTGCGGAGGGATCTC |
| | | CGTGGGGCGGTGAACGCCGATGATTATATAAGGACGCGCCGGGTG |
| | | TGGCACAGCTAGTTCCGTCGCAGCCGGGATTTGGGTCGCGGTTCTT |
| | | GTTTGTGGATCGCTGTGATCGTCACTTGGTGAGTTGCGGGCTGCTG |
| | | GGCTGGCCGGGGCTTTCGTGGCCGCCGGGCCGCTCGGTGGGACGG |
| | | AAGCGTGTGGAGAGACCGCCAAGGGCTGTAGTCTGGGTCCGCGAG |
| | | CAAGGTTGCCCTGAACTGGGGGTTGGGGGGAGCGCACAAAATGGC |
| | | GGCTGTTCCCGAGTCTTGAATGGAAGACGCTTGTAAGGCGGGCTG |
| hEF1aV1 | 26 | TGAGGTCGTTGAAACAAGGTGGGGGGCATGGTGGGCGGCAAGAA |
| | | CCCAAGGTCTTGAGGCCTTCGCTAATGCGGGAAAGCTCTTATTCGG |
| | | GTGAGATGGGCTGGGGCACCATCTGGGGACCCTGACGTGAAGTTT |
| | | GTCACTGACTGGAGAACTCGGGTTTGTCGTCTGGTTGCGGGGGCG |
| | | GCAGTTATGCGGTGCCGTTGGGCAGTGCACCCGTACCTTTGGGAGC |
| | | GCGCGCCTCGTCGTGTCGTGACGTCACCCGTTCTGTTGGCTTATAA |
| | | TGCAGGGTGGGGCCACCTGCCGGTAGGTGTGCGGTAGGCTTTTCTC |
| | | CGTCGCAGGACGCAGGGTTCGGGCCTAGGGTAGGCTCTCCTGAAT |
| | | CGACAGGCGCCGGACCTCTGGTGAGGGGAGGGATAAGTGAGGCGT |
| | | CAGTTTCTTTGGTCGGTTTTATGTACCTATCTTCTTAAGTAGCTGAA |
| | | GCTCCGGTTTTGAACTATGCGCTCGGGGTTGGCGAGTGTGTTTTGT |
| | | GAAGTTTTTTAGGCACCTTTTGAAATGTAATCATTTGGGTCAATAT |
| | | GTAATTTTCAGTGTTAGACTAGTAAAGCTTCTGCAGGTCGACTCTA |
| | | GAAAATTGTCCGCTAAATTCTGGCCGTTTTTGGCTTTTTTGTTAGAC |
| | | GGCTCCGGTGCCCGTCAGTGGGCAGAGCGCACATCGCCCACAGTC |
| | | CCCGAGAAGTTGGGGGGAGGGGTCGGCAATTGAACCGGTGCCTAG |
| | | AGAAGGTGGCGCGGGGTAAACTGGGAAAGTGATGTCGTGTACTGG |
| | | CTCCGCCTTTTTCCCGAGGGTGGGGGAGAACCGTATATAAGTGCA |
| | | GTAGTCGCCGTGAACGTTCTTTTTCGCAACGGGTTTGCCGCCAGAA |
| | | CACAGGTAAGTGCCGTGTGTGGTTCCCGCGGGCCTGGCCTCTTTAC |
| | | GGGTTATGGCCCTTGCGTGCCTTGAATTACTTCCACCTGGCTGCAG |
| | | TACGTGATTCTTGATCCCGAGCTTCGGGTTGGAAGTGGGTGGGAG |
| | | AGTTCGAGGCCTTGCGCTTAAGGAGCCCCTTCGCCTCGTGCTTGAG |
| | | TTGAGGCCTGGCCTGGGCGCTGGGGCCGCCGCGTGCGAATCTGGT |
| | | GGCACCTTCGCGCCTGTCTCGCTGCTTTCGATAAGTCTCTAGCCAT |
| | | TTAAAATTTTTGATGACCTGCTGCGACGCTTTTTTTCTGGCAAGAT |
| | | AGTCTTGTAAATGCGGGCCAAGATCTGCACACTGGTATTTCGGTTT |
| | | TTGGGGCCGCGGGCGGCGACGGGGCCCGTGCGTCCCAGCGCACAT |
| | | GTTCGGCGAGGCGGGGCCTGCGAGCGCGGCCACCGAGAATCGGAC |
| | | GGGGGTAGTCTCAAGCTGGCCGGCCTGCTCTGGTGCCTGGTCTCGC |
| | | GCCGCCGTGTATCGCCCCGCCCTGGGCGGCAAGGCTGGCCCGGTC |
| | | GGCACCAGTTGCGTGAGCGGAAAGATGGCCGCTTCCCGGCCCTGC |
| | | TGCAGGGAGCTCAAAATGGAGGACGCGGCGCTCGGGAGAGCGGG |
| | | CGGGTGAGTCACCCACACAAAGGAAAAGGGCCTTTCCGTCCTCAG |
| | | CCGTCGCTTCATGTGACTCCACGGAGTACCGGGCGCCGTCCAGGC |
| | | ACCTCGATTAGTTCTCGAGCTTTTGGAGTACGTCGTCTTTAGGTTG |
| | | GGGGGAGGGGTTTTATGCGATGGAGTTTCCCCACACTGAGTGGGT |
| | | GGAGACTGAAGTTAGGCCAGCTTGGCACTTGATGTAATTCTCCTTG |
| | | GAATTTGCCCTTTTTGAGTTTGGATCTTGGTTCATTCTCAAGCCTCA |
| | | GACAGTGGTTCAAAGTTTTTTTCTTCCATTTCAGGTGTCGTGA |
| hCAGG | 27 | ACTAGTTATTAATAGTAATCAATTACGGGGTCATTAGTTCATAGCC |
| | | CATATATGGAGTTCCGCGTTACATAACTTACGGTAAATGGCCCGCC |
| | | TGGCTGACCGCCCAACGACCCCCGCCCATTGACGTCAATAATGAC |
| | | GTATGTTCCCATAGTAACGCCAATAGGGACTTTCCATTGACGTCAA |
| | | TGGGTGGAGTATTTACGGTAAACTGCCCACTTGGCAGTACATCAA |
| | | GTGTATCATATGCCAAGTACGCCCCCTATTGACGTCAATGACGGTA |
| | | AATGGCCCGCCTGGCATTATGCCCAGTACATGACCTTATGGGACTT |
| | | TCCTACTTGGCAGTACATCTACGTATTAGTCATCGCTATTACCATG |
| | | GTCGAGGTGAGCCCCACGTTCTGCTTCACTCTCCCCATCTCCCCCC |
| | | CCTCCCCACCCCCAATTTTGTATTTATTTATTTTTTAATTATTTTGTG |
| | | CAGCGATGGGGGCGGGGGGGGGGGGGGGGCGCGCGCCAGGCGGG |
| | | GCGGGGCGGGGCGAGGGGCGGGGCGGGGCGAGGCGGAGAGGTGC |
| | | GGCGGCAGCCAATCAGAGCGGCGCGCTCCGAAAGTTTCCTTTTAT |
| | | GGCGAGGCGGCGGCGGCGGCGGCCCTATAAAAAGCGAAGCGCGC |
| | | GGCGGGCGGGGAGTCGCTGCGACGCTGCCTTCGCCCCGTGCCCCG |
| | | CTCCGCCGCCGCCTCGCGCCGCCCGCCCCGGCTCTGACTGACCGCG |
| | | TTACTCCCACAGGTGAGCGGGCGGGACGGCCCTTCTCCTCCGGGCT |
| | | GTAATTAGCGCTTGGTTTAATGACGGCTTGTTTCTTTTCTGTGGCTG |
| | | CGTGAAAGCCTTGAGGGGCTCCGGGAGGGCCCTTTGTGCGGGGGG |
| | | AGCGGCTCGGGGGGTGCGTGCGTGTGTGTGTGCGTGGGGAGCGCC |
| | | GCGTGCGGCTCCGCGCTGCCCGGCGGCTGTGAGCGCTGCGGGCGC |
| | | GGCGCGGGGCTTTGTGCGCTCCGCAGTGTGCGCGAGGGGAGCGCG |
| | | GCCGGGGGCGGTGCCCCGCGGTGCGGGGGGGGCTGCGAGGGGAA |
| | | CAAAGGCTGCGTGCGGGGTGTGTGCGTGGGGGGGTGAGCAGGGG |
| | | GTGTGGGCGCGTCGGTCGGGCTGCAACCCCCCCTGCACCCCCCTCC |
| | | CCGAGTTGCTGAGCACGGCCCGGCTTCGGGTGCGGGGCTCCGTAC |
| | | GGGGCGTGGCGCGGGGCTCGCCGTGCCGGGCGGGGGGTGGCGGC |
| | | AGGTGGGGGTGCCGGGCGGGGCGGGGCCGCCTCGGGCCGGGGAG |
| | | GGCTCGGGGGAGGGGCGCGGCGGCCCCCGGAGCGCCGGCGGCTG |
| | | TCGAGGCGCGGCGAGCCGCAGCCATTGCCTTTTATGGTAATCGTGC |
| | | GAGAGGGCGCAGGGACTTCCTTTGTCCCAAATCTGTGCGGAGCCG |
| | | AAATCTGGGAGGCGCCGCCGCACCCCCTCTAGCGGGCGCGGGGCG |
| | | AAGCGGTGCGGCGCCGGCAGGAAGGAAATGGGCGGGGAGGGCCT |
| | | TCGTGCGTCGCCGCGCCGCCGTCCCCTTCTCCCTCTCCAGCCTCGG |
| | | GGCTGTCCGCGGGGGGACGGCTGCCTTCGGGGGGGACGGGGCAGG |
| | | GCGGGGTTCGGCTTCTGGCGTGTGACCGGCGGCTCTAGAGCCTCTG |
| | | CTAACCATGTTCATGCCTTCTTCTTTTTCCTACAGCTCCTGGGCAAC |
| | | GTGCTGGTTATTGTGCTGTCTCATCATTTTGGCAAAGAATTC |
| hEF1aV2 | 28 | GGGCAGAGCGCACATCGCCCACAGTCCCCGAGAAGTTGGGGGGAG |
| | | GGGTCGGCAATTGAACCGGTGCCTAGAGAAGGTGGCGCGGGGTAA |
| | | ACTGGGAAAGTGATGTCGTGTACTGGCTCCGCCTTTTTCCCGAGGG |
| | | TGGGGGAGAACCGTATATAAGTGCAGTAGTCGCCGTGAACGTTCT |
| | | TTTTCGCAACGGGTTTGCCGCCAGAACACAG |
| hACTb | 29 | CCACTAGTTCCATGTCCTTATATGGACTCATCTTTGCCTATTGCGAC |
| | | ACACACTCAATGAACACCTACTACGCGCTGCAAAGAGCCCCGCAG |
| | | GCCTGAGGTGCCCCCACCTCACCACTCTTCCTATTTTTGTGTAAAA |
| | | ATCCAGCTTCTTGTCACCACCTCCAAGGAGGGGGAGGAGGAGGAA |
| | | GGCAGGTTCCTCTAGGCTGAGCCGAATGCCCCTCTGTGGTCCCACG |
| | | CCACTGATCGCTGCATGCCCACCACCTGGGTACACACAGTCTGTGA |
| | | TTCCCGGAGCAGAACGGACCCTGCCCACCCGGTCTTGTGTGCTACT |
| | | CAGTGGACAGACCCAAGGCAAGAAAGGGTGACAAGGACAGGGTC |
| | | TTCCCAGGCTGGCTTTGAGTTCCTAGCACCGCCCCGCCCCCAATCC |
| | | TCTGTGGCACATGGAGTCTTGGTCCCCAGAGTCCCCCAGCGGCCTC |
| | | CAGATGGTCTGGGAGGGCAGTTCAGCTGTGGCTGCGCATAGCAGA |
| | | CATACAACGGACGGTGGGCCCAGACCCAGGCTGTGTAGACCCAGC |
| | | CCCCCCGCCCCGCAGTGCCTAGGTCACCCACTAACGCCCCAGGCCT |
| | | GGTCTTGGCTGGGCGTGACTGTTACCCTCAAAAGCAGGCAGCTCC |
| | | AGGGTAAAAGGTGCCCTGCCCTGTAGAGCCCACCTTCCTTCCCAGG |
| | | GCTGCGGCTGGGTAGGTTTGTAGCCTTCATCACGGGCCACCTCCAG |
| | | CCACTGGACCGCTGGCCCCTGCCCTGTCCTGGGGAGTGTGGTCCTG |
| | | CGACTTCTAAGTGGCCGCAAGCCACCTGACTCCCCCAACACCACA |
| | | CTCTACCTCTCAAGCCCAGGTCTCTCCCTAGTGACCCACCCAGCAC |
| | | ATTTAGCTAGCTGAGCCCCACAGCCAGAGGTCCTCAGGCCCTGCTT |
| | | TCAGGGCAGTTGCTCTGAAGTCGGCAAGGGGGAGTGACTGCCTGG |
| | | CCACTCCATGCCCTCCAAGAGCTCCTTCTGCAGGAGCGTACAGAA |
| | | CCCAGGGCCCTGGCACCCGTGCAGACCCTGGCCCACCCCACCTGG |
| | | GCGCTCAGTGCCCAAGAGATGTCCACACCTAGGATGTCCCGCGGT |
| | | GGGTGGGGGGCCCGAGAGACGGGCAGGCCGGGGGCAGGCCTGGC |
| | | CATGCGGGGCCGAACCGGGCACTGCCCAGCGTGGGGCGCGGGGG |
| | | CCACGGCGCGCGCCCCCAGCCCCCGGGCCCAGCACCCCAAGGCGG |
| | | CCAACGCCAAAACTCTCCCTCCTCCTCTTCCTCAATCTCGCTCTCGC |
| | | TCTTTTTTTTTTTCGCAAAAGGAGGGGAGAGGGGGTAAAAAAATG |
| | | CTGCACTGTGCGGCGAAGCCGGTGAGTGAGCGGCGCGGGGCCAAT |
| | | CAGCGTGCGCCGTTCCGAAAGTTGCCTTTTATGGCTCGAGCGGCCG |
| | | CGGCGGCGCCCTATAAAACCCAGCGGCGCGACGCGCCACCACCGC |
| | | CGAGACCGCGTCCGCCCCGCGAGCACAGAGCCTCGCCTTTGCCGA |
| | | TCCGCCGCCCGTCCACACCCGCCGCCAGGTAAGCCCGGCCAGCCG |
| | | ACCGGGGCAGGCGGCTCACGGCCCGGCCGCAGGCGGCCGCGGCCC |
| | | CTTCGCCCGTGCAGAGCCGCCGTCTGGGCCGCAGCGGGGGGCGCA |
| | | TGGGGGGGGAACCGGACCGCCGTGGGGGGCGCGGGAGAAGCCCC |
| | | TGGGCCTCCGGAGATGGGGGACACCCCACGCCAGTTCGGAGGCGC |
| | | GAGGCCGCGCTCGGGAGGCGCGCTCCGGGGGTGCCGCTCTCGGGG |
| | | CGGGGGCAACCGGCGGGGTCTTTGTCTGAGCCGGGCTCTTGCCAA |
| | | TGGGGATCGCAGGGTGGGCGCGGCGGAGCCCCCGCCAGGCCCGGT |
| | | GGGGGCTGGGGCGCCATTGCGCGTGCGCGCTGGTCCTTTGGGCGC |
| | | TAACTGCGTGCGCGCTGGGAATTGGCGCTAATTGCGCGTGCGCGCT |
| | | GGGACTCAAGGCGCTAACTGCGCGTGCGTTCTGGGGCCCGGGGTG |
| | | CCGCGGCCTGGGCTGGGGCGAAGGCGGGCTCGGCCGGAAGGGGT |
| | | GGGGTCGCCGCGGCTCCCGGGCGCTTGCGCGCACTTCCTGCCCGA |
| | | GCCGCTGGCCGCCCGAGGGTGTGGCCGCTGCGTGCGCGCGCGCCG |
| | | ACCCGGCGCTGTTTGAACCGGGCGGAGGCGGGGCTGGCGCCCGGT |
| | | TGGGAGGGGGTTGGGGCCTGGCTTCCTGCCGCGCGCCGCGGGGAC |
| | | GCCTCCGACCAGTGTTTGCCTTTTATGGTAATAACGCGGCCGGCCC |
| | | GGCTTCCTTTGTCCCCAATCTGGGCGCGCGCCGGCGCCCCCTGGCG |
| | | GCCTAAGGACTCGGCGCGCCGGAAGTGGCCAGGGCGGGGGCGAC |
| | | CTCGGCTCACAGCGCGCCCGGCTAT |
| heIF4A1 | 30 | GTTGATTTCCTTCATCCCTGGCACACGTCCAGGCAGTGTCGAATCC |
| | | ATCTCTGCTACAGGGGAAAACAAATAACATTTGAGTCCAGTGGAG |
| | | ACCGGGAGCAGAAGTAAAGGGAAGTGATAACCCCCAGAGCCCGG |
| | | AAGCCTCTGGAGGCTGAGACCTCGCCCCCCTTGCGTGATAGGGCCT |
| | | ACGGAGCCACATGACCAAGGCACTGTCGCCTCCGCACGTGTGAGA |
| | | GTGCAGGGCCCCAAGATGGCTGCCAGGCCTCGAGGCCTGACTCTT |
| | | CTATGTCACTTCCGTACCGGCGAGAAAGGCGGGCCCTCCAGCCAA |
| | | TGAGGCTGCGGGGGGGGCCTTCACCTTGATAGGCACTCGAGTTATC |
| | | CAATGGTGCCTGCGGGCCGGAGCGACTAGGAACTAACGTCATGCC |
| | | GAGTTGCTGAGCGCCGGCAGGCGGGGCCGGGGCGGCCAAACCAAT |
| | | GCGATGGCCGGGGCGGAGTCGGGCGCTCTATAAGTTGTCGATAGG |
| | | CGGGCACTCCGCCCTAGTTTCTAAGGACCATG |
| hGAPDH | 31 | AGTTCCCCAACTTTCCCGCCTCTCAGCCTTTGAAAGAAAGAAAGGG |
| | | GAGGGGGCAGGCCGCGTGCAGTCGCGAGCGGTGCTGGGCTCCGGC |
| | | TCCAATTCCCCATCTCAGTCGCTCCCAAAGTCCTTCTGTTTCATCCA |
| | | AGCGTGTAAGGGTCCCCGTCCTTGACTCCCTAGTGTCCTGCTGCCC |
| | | ACAGTCCAGTCCTGGGAACCAGCACCGATCACCTCCCATCGGGCC |
| | | AATCTCAGTCCCTTCCCCCCTACGTCGGGGCCCACACGCTCGGTGC |
| | | GTGCCCAGTTGAACCAGGCGGCTGCGGAAAAAAAAAAGCGGGGA |
| | | GAAAGTAGGGCCCGGCTACTAGCGGTTTTACGGGCGCACGTAGCT |
| | | CAGGCCTCAAGACCTTGGGCTGGGACTGGCTGAGCCTGGCGGGAG |
| | | GCGGGGTCCGAGTCACCGCCTGCCGCCGCGCCCCCGGTTTCTATAA |
| | | ATTGAGCCCGCAGCCTCCCGCTTCGCTCTCTGCTCCTCCTGTTCGA |
| | | CAGTCAGCCGCATCTTCTTTTGCGTCGCCAGGTGAAGACGGGCGG |
| | | AGAGAAACCCGGGAGGCTAGGGACGGCCTGAAGGCGGCAGGGGC |
| | | GGGCGCAGGCCGGATGTGTTCGCGCCGCTGCGGGGTGGGCCCGGG |
| | | CGGCCTCCGCATTGCAGGGGGGGGCGGAGGACGTGATGCGGCGCG |
| | | GGCTGGGCATGGAGGCCTGGTGGGGGAGGGGAGGGGAGGCGTGG |
| | | GTGTCGGCCGGGGCCACTAGGCGCTCACTGTTCTCTCCCTCCGCGC |
| | | AGCCGAGCCACATCGCTGAGACAC |
| hGRP78 | 157 | AGTGCGGTTACCAGCGGAAATGCCTCGGGGTCAGAAGTCGCAGGA |
| | | GAGATAGACAGCTGCTGAACCAATGGGACCAGCGGATGGGGCGG |
| | | ATGTTATCTACCATTGGTGAACGTTAGAAACGAATAGCAGCCAAT |
| | | GAATCAGCTGGGGGGGCGGAGCAGTGACGTTTATTGCGGAGGGGG |
| | | CCGCTTCGAATCGGCGGCGGCCAGCTTGGTGGCCTGGGCCAATGA |
| | | ACGGCCTCCAACGAGCAGGGCCTTCACCAATCGGCGGCCTCCACG |
| | | ACGGGGCTGGGGGAGGGTATATAAGCCGAGTAGGCGACGGTGAG |
| | | GTCGACGCCGGCCAAGACAGCACAGACAGATTGACCTATTGGGGT |
| | | GTTTCGCGAGTGTGAGAGGGAAGCGCCGCGGCCTGTATTTCTAGA |
| | | CCTGCCCTTCGCCTGGTTCGTGGCGCCTTGTGACCCCGGGCCCCTG |
| | | CCGCCTGCAAGTCGGAAATTGCGCTGTGCTCCTGTGCTACGGCCTG |
| | | TGGCTGGACTGCCTGCTGCTGCCCAACTGGCTGGCAC |
| hGRP94 | 33 | TAGTTTCATCACCACCGCCACCCCCCCGCCCCCCCGCCATCTGAAA |
| | | GGGTTCTAGGGGATTTGCAACCTCTCTCGTGTGTTTCTTCTTTCCGA |
| | | GAAGCGCCGCCACACGAGAAAGCTGGCCGCGAAAGTCGTGCTGGA |
| | | ATCACTTCCAACGAAACCCCAGGCATAGATGGGAAAGGGTGAAGA |
| | | ACACGTTGCCATGGCTACCGTTTCCCCGGTCACGGAATAAACGCTC |
| | | TCTAGGATCCGGAAGTAGTTCCGCCGCGACCTCTCTAAAAGGATG |
| | | GATGTGTTCTCTGCTTACATTCATTGGACGTTTTCCCTTAGAGGCCA |
| | | AGGCCGCCCAGGCAAAGGGGCGGTCCCACGCGTGAGGGGCCCGC |
| | | GGAGCCATTTGATTGGAGAAAAGCTGCAAACCCTGACCAATCGGA |
| | | AGGAGCCACGCTTCGGGCATCGGTCACCGCACCTGGACAGCTCCG |
| | | ATTGGTGGACTTCCGCCCCCCCTCACGAATCCTCATTGGGTGCCGT |
| | | GGGTGCGTGGTGCGGCGCGATTGGTGGGTTCATGTTTCCCGTCCCC |
| | | CGCCCGCGAGAAGTGGGGGTGAAAAGCGGCCCGACCTGCTTGGGG |
| | | TGTAGTGGGCGGACCGCGCGGCTGGAGGTGTGAGGATCCGAACCC |
| | | AGGGGTGGGGGGTGGAGGCGGCTCCTGCGATCGAAGGGGACTTGA |
| | | GACTCACCGGCCGCACGTC |
| hHSP70 | 34 | GGGCCGCCCACTCCCCCTTCCTCTCAGGGTCCCTGTCCCCTCCAGT |
| | | GAATCCCAGAAGACTCTGGAGAGTTCTGAGCAGGGGGCGGCACTC |
| | | TGGCCTCTGATTGGTCCAAGGAAGGCTGGGGGGCAGGACGGGAGG |
| | | CGAAAACCCTGGAATATTCCCGACCTGGCAGCCTCATCGAGCTCG |
| | | GTGATTGGCTCAGAAGGGAAAAGGCGGGTCTCCGTGACGACTTAT |
| | | AAAAGCCCAGGGGCAAGCGGTCCGGATAACGGCTAGCCTGAGGA |
| | | GCTGCTGCGACAGTCCACTACCTTTTTCGAGAGTGACTCCCGTTGT |
| | | CCCAAGGCTTCCCAGAGCGAACCTGTGCGGCTGCAGGCACCGGCG |
| | | CGTCGAGTTTCCGGCGTCCGGAAGGACCGAGCTCTTCTCGCGGATC |
| | | CAGTGTTCCGTTTCCAGCCCCCAATCTCAGAGCGGAGCCGACAGA |
| | | GAGCAGGGAACCC |
| hKINb | 35 | GCCCCACCCCCGTCCGCGTTACAACCGGGAGGCCCGCTGGGTCCT |
| | | GCACCGTCACCCTCCTCCCTGTGACCGCCCACCTGATACCCAAACA |
| | | ACTTTCTCGCCCCTCCAGTCCCCAGCTCGCCGAGCGCTTGCGGGGA |
| | | GCCACCCAGCCTCAGTTTCCCCAGCCCCGGGCGGGGCGAGGGGCG |
| | | ATGACGTCATGCCGGCGCGCGGCATTGTGGGGCGGGGCGAGGCGG |
| | | GGCGCCGGGGGGAGCAACACTGAGACGCCATTTTCGGCGGCGGGA |
| | | GCGGCGCAGGCGGCCGAGCGGGACTGGCTGGGTCGGCTGGGCTGC |
| | | TGGTGCGAGGAGCCGCGGGGCTGTGCTCGGCGGCCAAGGGGACAG |
| | | CGCGTGGGTGGCCGAGGATGCTGCGGGGCGGTAGCTCCGGCGCCC |
| | | CTCGCTGGTGACTGCTGCGCCGTGCCTCACACAGCCGAGGCGGGC |
| | | TCGGCGCACAGTCGCTGCTCCGCGCTCGCGCCCGGCGGCGCTCCA |
| | | GGTGCTGACAGCGCGAGAGAGCGCGGCCTCAGGAGCAACAC |
| hUBIb | 36 | TTCCAGAGCTTTCGAGGAAGGTTTCTTCAACTCAAATTCATCCGCC |
| | | TGATAATTTTCTTATATTTTCCTAAAGAAGGAAGAGAAGCGCATAG |
| | | AGGAGAAGGGAAATAATTTTTTAGGAGCCTTTCTTACGGCTATGA |
| | | GGAATTTGGGGCTCAGTTGAAAAGCCTAAACTGCCTCTCGGGAGG |
| | | TTGGGCGCGGCGAACTACTTTCAGCGGCGCACGGAGACGGCGTCT |
| | | ACGTGAGGGGTGATAAGTGACGCAACACTCGTTGCATAAATTTGC |
| | | GCTCCGCCAGCCCGGAGCATTTAGGGGCGGTTGGCTTTGTTGGGTG |
| | | AGCTTGTTTGTGTCCCTGTGGGTGGACGTGGTTGGTGATTGGCAGG |
| | | ATCCTGGTATCCGCTAACAGGTACTGGCCCACAGCCGTAAAGACC |
| | | TGCGGGGGCGTGAGAGGGGGGAATGGGTGAGGTCAAGCTGGAGG |
| | | CTTCTTGGGGTTGGGTGGGCCGCTGAGGGGAGGGGAGGGCGAGGT |
| | | GACGCGACACCCGGCCTTTCTGGGAGAGTGGGCCTTGTTGACCTA |
| | | AGGGGGGCGAGGGCAGTTGGCACGCGCACGCGCCGACAGAAACT |
| | | AACAGACATTAACCAACAGCGATTCCGTCGCGTTTACTTGGGAGG |
| | | AAGGCGGAAAAGAGGTAGTTTGTGTGGCTTCTGGAAACCCTAAAT |
| | | TTGGAATCCCAGTATGAGAATGGTGTCCCTTCTTGTGTTTCAATGG |
| | | GATTTTTACTTCGCGAGTCTTGTGGGTTTGGTTTTGTTTTCAGTTTG |
| | | CCTAACACCGTGCTTAGGTTTGAGGCAGATTGGAGTTCGGTCGGG |
| | | GGAGTTTGAATATCCGGAACAGTTAGTGGGGAAAGCTGTGGACGC |
| | | TTGGTAAGAGAGCGCTCTGGATTTTCCGCTGTTGACGTTGAAACCT |
| | | TGAATGACGAATTTCGTATTAAGTGACTTAGCCTTGTAAAATTGAG |
| | | GGGAGGCTTGCGGAATATTAACGTATTTAAGGCATTTTGAAGGAA |
| | | TAGTTGCTAATTTTGAAGAATATTAGGTGTAAAAGCAAGAAATAC |
| | | AATGATCCTGAGGTGACACGCTTATGTTTTACTTTTAAACTAGGTC |
| | | ACC |
| ZF binding site | 37 | cgggtttcgtaacaatcgcatgaggattcgcaacgccttcGGCGTAGCCGATGTCGCGctccc |
| | | gtctcagtaaaggtcGGCGTAGCCGATGTCGCGcaatcggactgccttcgtacGGCGTA |
| | | GCCGATGTCGCGcgtatcagtcgcctcggaacGGCGTAGCCGATGTCGCGcattc |
| | | gtaagaggctcactctcccttacacggagtggataACTAGTTCTAGAGGGTATATAATG |
| | | GGGGCCA |
| Exemplary | 38 | TCTGTTCCTGTTAATCAACCTCTGGATTACAAAATTTGTGAAAGAT |
| WPRE | | TGACTGATATTCTTAACTATGTTGCTCCTTTTACGCTGTGTGGATAT |
| | | GCTGCTTTAATGCCTCTGTATCATGCTATTGCTTCCCGTACGGCTTT |
| | | CGTTTTCTCCTCCTTGTATAAATCCTGGTTGCTGTCTCTTTATGAGG |
| | | AGTTGTGGCCCGTTGTCCGTCAACGTGGCGTGGTGTGCTCTGTGTT |
| | | TGCTGACGCAACCCCCACTGGCTGGGGCATTGCCACCACCTGTCA |
| | | ACTCCTTTCTGGGACTTTCGCTTTCCCCCTCCCGATCGCCACGGCA |
| | | GAACTCATCGCCGCCTGCCTTGCCCGCTGCTGGACAGGGGCTAGGT |
| | | TGCTGGGCACTGATAATTCCGTGGTGTTGTCGGGGAAGCTGACGTC |
| | | CTTTCCATGGCTGCTCGCCTGTGTTGCCAACTGGATCCTGCGCGGG |
| | | ACGTCCTTCTGCTACGTCCCTTCGGCTCTCAATCCAGCGGACCTCC |
| | | CTTCCCGAGGCCTTCTGCCGGTTCTGCGGCCTCTCCCGCGTCTTCG |
| | | CTTTCGGCCTCCGACGAGTCGGATCTCCCTTTGGGCCGCCTCCCCG |
| | | CCTGTTTCGCCTCGGCGTCCGGTCCGTGTTGCTTGGTCGTCACCTGT |
| | | GCAGAATTGCGAACCATGGATTCCA |
| Exemplary | 169 | TCTGTTCCTGTTAATCAACCTCTGGATTACAAAATTTGTGAAAGAT |
| WPRE | | TGACTGATATTCTTAACTATGTTGCTCCTTTTACGCTGTGTGGATAT |
| | | GCTGCTTTAATGCCTCTGTATCATGCTATTGCTTCCCGTACGGCTTT |
| | | CGTTTTCTCCTCCTTGTATAAATCCTGGTTGCTGTCTCTTTATGAGG |
| | | AGTTGTGGCCCGTTGTCCGTCAACGTGGCGTGGTGTGCTCTGTGTT |
| | | TGCTGACGCAACCCCCACTGGCTGGGGCATTGCCACCACCTGTCA |
| | | ACTCCTTTCTGGGACTTTCGCTTTCCCCCTCCCGATCGCCACGGCA |
| | | GAACTCATCGCCGCCTGCCTTGCCCGCTGCTGGACAGGGGCTAGGT |
| | | TGCTGGGCACTGATAATTCCGTGGTGTTGTCGGGGAAATCATCGTC |
| | | CTTTCCTTGGCTGCTCGCCTGTGTTGCCAACTGGATCCTGCGCGGG |
| | | ACGTCCTTCTGCTACGTCCCTTCGGCTCTCAATCCAGCGGACCTCC |
| | | CTTCCCGAGGCCTTCTGCCGGTTCTGCGGCCTCTCCCGCGTCTTCG |
| | | CTTTCGGCCTCCGACGAGTCGGATCTCCCTTTGGGCCGCCTCCCCG |
| | | CCTGTTTCGCCTCGGCGTCCGGTCCGTGTTGCTTGGTCGTCACCTGT |
| | | GCAGAATTGCGAACCATGGATTCC |
| Wild Type | 40 | AGDMRAANLWPSPLMIKRSKKNSLALSLTADQMVSALLDAEPPILYS |
| ERT2 | | EYDPTRPFSEASMMGLLTNLADRELVHMINWAKRVPGFVDLTLHDQ |
| “ERT2 WT” | | VHLLECAWLEILMIGLVWRSMEHPVKLLFAPNLLLDRNQGKCVEGM |
| as included in | | VEIFDMLLATSSRFRMMNLQGEEFVCLKSIILLNSGVYTFLSSTLKSLE |
| SB03422 | | EKDHIHRVLDKITDTLIHLMAKAGLTLQQQHQRLAQLLLILSHIRHMS |
| (corresponding | | NKGMEHLYSMKCKNVVPLYDLLLEAADAHRLHAPTSRGGASVEETD |
| to positions | | QSHLATAGSTSSHSLQKYYITGEAEGFPAT |
| 283-594 of | | |
| SEQ ID NO: 1) | | |
| ERT2 mutant | 66 | AGDMRAANLWPSPLMIKRSKKNSLALSLTADQMVSALLDAEPPILYS |
| 81, as included | | EYDPTRPFSEASMMGLLTNLADRELVHMINWAKRVPGFVDLTLHDQ |
| in SB06136 | | VHLLECAWMEILMIGVVWRSMEHPVKLLFAPNLLLDRDQGKCVEGL |
| (corresponding | | VEIFDMLLATSSRFRMMNLQGEEFVCLKSIILLNSGVYTFLPSTLKSLE |
| to positions | | EKDHIHRVLDKITDTLIHLMAKAGLTLQQQHQRLAQLLLILSHIRHMS |
| 283-594 of | | NKGMELLYSMKCKNVVPLYDLLLEAADAHRLHAPTSRGGASVEETD |
| SEQ ID NO: 1) | | QSHLATAGSTSSHSLQKYYITGEAEGFPAT |
| L384M/L391V/ | | |
| N413D/ | | |
| M421L/S463P/ | | |
| H524L | | |
| ERT2 mutant | 170 | AGDMRAANLWPSPLMIKRSKKNSLALSLTADQMVSALLDAEPPILYS |
| 93, as included | | EYDPTRPFSEASMMGLLTNLADRELVHMINWAKRVPGFVDLTLHDQ |
| in SB06138 | | VHLLECAWMEILMIGLVWRSMEHPVKLLFAPNLVLDRDQGKCVEGM |
| L384M/ | | VEIFDMLLATSSRFRMMNLQGEEFVCLKSIILLNSGVYTFLPSTLKSLE |
| L409V/ | | EKDHIHRVLDKITDTLIHLMAKAGLTLQQQHQRLAQLLLILSHIRHMS |
| N413D/S463P/ | | NKGMELLYSMKCKNVVPLYDLLLEAADAHRLHAPTSRGGASVEETD |
| H524L | | QSHLATAGSTSSHSLQKYYITGEAEGFPAT |
| ERT2 mutant | 43 | AGDMRAANLWPSPLMIKRSKKNSLALSLTADQMVSALLDAEPPILYS |
| 86, as included | | EYDPTRPFSEASMMGLLTNLADREIVHMINWAKRVPGFVDLTLHDQV |
| in SB06139 | | HLLECAWMEILMIGVVWRSMEHPVKLLFAPNLLLDRNQGKCVEGMV |
| L354I/L384M/ | | EIFDMLLATSSRFRMMNLQGEEFVCLKSIILLNSGVYTFLPSTLKSLEE |
| L391V/ | | KDHIHRVLDKITDTLIHLMAKAGLTLQQQHQRLAQLLLILSHIRHMSN |
| S463P | | KGMEHLYSMKCKNVVPLYDLLLEAADAHRLHAPTSRGGASVEETDQ |
| | | SHLATAGSTSSHSLQKYYITGEAEGFPAT |
| ERT2 mutant | 44 | AGDMRAANLWPSPLMIKRSKKNSLALSLTADQMVSALLDAEPPILYS |
| 95, as included | | EYDPTRPFSEASMMGLLTNLADREIVHMINWAKRVPGFVDLTLHDQV |
| in SB06140 | | HLLECAWMEILMIGLVWRSMEHPVKLLFAPNLVLDRDQGKCVEGLV |
| L354I/L384M/ | | EIFDMLLATSSRFRMMNLQGEEFVCLKSIILLNSGVYTFLPSTLKSLEE |
| L409V/ | | KDHIHRVLDKITDTLIHLMAKAGLTLQQQHQRLAQLLLILSHIRHMSN |
| N413D/ | | KGMEFLYSMKCKNVVPLYDLLLEAADAHRLHAPTSRGGASVEETDQ |
| M421L/S463P/ | | SHLATAGSTSSHSLQKYYITGEAEGFPAT |
| H524F | | |
| ERT2 mutant | 171 | AGDMRAANLWPSPLMIKRSKKNSLALSLTADQMVSALLDAEPPILYS |
| 88, as included | | EYDPTRPFSEASMMGLLTNLADRELVHMINWAKRVPGFVDLTLHDQ |
| in SB06141 | | VHLLECAWLEILMIGVVWRSMEHPVKLLFAPNLLLDRDEGKCVEGM |
| L391V/ | | VEIFDMLLATSSRFRMMNLQGEEFVCLKSIILLNSGVYTFLPSTLKSLE |
| N413D/ | | EKDHIHR VLDKITDTLIHLMAKAGLTLQQQHQRLAQLLLILSHIRHMS |
| Q414E/S463P/ | | NKGMEFLYSMKCKNVVPLYDLLLEAADAHRLHAPTSRGGASVEETD |
| H524F | | QSHLATAGSTSSHSLQKYYITGEAEGFPAT |
| ERT2 mutant | 172 | AGDMRAANLWPSPLMIKRSKKNSLALSLTADQMVSALLDAEPPILYS |
| 77, as included | | EYDPTRPFSEASMMGLLTNLADREIVHMINWAKRVPGFVDLTLHDQV |
| in SB06142 | | HLLECAWLEILMIGVVWRSMEHPVKLLFAPNLLLDRDQGKCVEGLV |
| L354I/ L391V/ | | EIFDMLLATSSRFRMMNLQGEEFVCLKSIILLNSGVYTFLPSTLKSLEE |
| N413D/ | | KDHIHRVLDKITDTLIHLMAKAGLTLQQQHQRLAQLLLILSHIRHASN |
| M421L/S463P/ | | KGMELLYSMKCKNVVPLYDLLLEAADAHRLHAPTSRGGASVEETDQ |
| M517A/ | | SHLATAGSTSSHSLQKYYITGEAEGFPAT |
| H524L | | |
| ERT2 mutant | 173 | AGDMRAANLWPSPLMIKRSKKNSLALSLTADQMVSALLDAEPPILYS |
| 49, as included | | EYDPTRPFSEASMMGLLTNLADRELVHMINWAKRVPGFVDLTLHDQ |
| in SB06143 | | VHLLECAWLEILMIGVVWRSMEHPVKLLFAPNLLLDRNEGKCVEGL |
| L391V/Q414E/ | | VEIFDMLLATSSRFRMMNLQGEEFVCLKSIILLNSGVYTFLPSTLKSLE |
| M421L/ | | EKDHIHRVLDKITDTLIHLMAKAGLTLQQQHQRLAQLLLILSHIRHMS |
| S463P/H524F | | NKGMEFLYSMKCKNVVPLYDLLLEAADAHRLHAPTSRGGASVEETD |
| | | QSHLATAGSTSSHSLQKYYITGEAEGFPAT |
| ERT2 mutant | 174 | AGDMRAANLWPSPLMIKRSKKNSLALSLTADQMVSALLDAEPPILYS |
| 58, as included | | EYDPTRPFSEASMMGLLTNLADREIVHMINWAKRVPGFVDLTLHDQV |
| in SB06144 | | HLLECAWLEILMIGLVWRSMEHPVKLLFAPNLVLDRDQGKCVEGLV |
| L354I/L409V/ | | EIFDMLLATSSRFRMMNLQGEEFVCLKSIILLNSGVYTFLSSTLKSLEE |
| N413D/ | | KDHIHRVLDKITDTLIHLMAKAGLTLQQQHQRLAQLLLILSHIRHMSN |
| M421L/ | | KGMELLYSMKCKNVVPLYDLLLEAADAHRLHAPTSRGGASVEETDQ |
| H524L | | SHLATAGSTSSHSLQKYYITGEAEGFPAT |
| ERT2 mutant | 49 | AGDMRAANLWPSPLMIKRSKKNSLALSLTADQMVSALLDAEPPILYS |
| 62, as included | | EYDPTRPFSEASMMGLLTNLADRELVHMINWAKRVPGFVDLTLHDQ |
| in SB06145 | | VHLLECAWLEILMIGLVWRSMEHPVKLLFAPNLVLDRDEGKCVEGL |
| L409V/ | | VEIFDMLLATSSRFRMMNLQGEEFVCLKSIILLNSGVYTFLPSTLKSLE |
| N413D/ | | EKDHIHRVLDKITDTLIHLMAKAGLTLQQQHQRLAQLLLILSHIRHMS |
| Q414E/ | | NKGMELLYSMKCKNVVPLYDLLLEAADAHRLHAPTSRGGASVEETD |
| M421L/S463P/ | | QSHLATAGSTSSHSLQKYYITGEAEGFPAT |
| H524L | | |
| ERT2 mutant | 50 | AGDMRAANLWPSPLMIKRSKKNSLALSLTADQMVSALLDAEPPILYS |
| 63, as included | | EYDPTRPFSEASMMGLLTNLADREIVHMINWAKRVPGFVDLTLHDQV |
| in SB06146 | | HLLECAWLEILMIGVVWRSMEHPVKLLFAPNLLLDRDEGKCVEGLVE |
| L354I/L391V/ | | IFDMLLATSSRFRMMNLQGEEFVCLKSIILLNSGVYTFLSSTLKSLEEK |
| N413D/ | | DHIHRVLDKITDTLIHLMAKAGLTLQQQHQRLAQLLLILSHIRHASNK |
| Q414E/ | | GMEFLYSMKCKNVVPLYDLLLEAADAHRLHAPTSRGGASVEETDQS |
| M421L/ | | HLATAGSTSSHSLQKYYITGEAEGFPAT |
| M517A/ | | |
| H524F | | |
| ERT2 mutant | 51 | AGDMRAANLWPSPLMIKRSKKNSLALSLTADQMVSALLDAEPPILYS |
| 55, as included | | EYDPTRPFSEASMMGLLTNLADREIVHMINWAKRVPGFVDLTLHDQV |
| in SB06147 | | HLLECAWLEILMIGLVWRSMEHPVKLLFAPNLVLDRNQGKCVEGLV |
| L354I/L409V/ | | EIFDMLLATSSRFRMMNLQGEEFVCLKSIILLNSGVYTFLPSTLKSLEE |
| M421L/ | | KDHIHRVLDKITDTLIHLMAKAGLTLQQQHQRLAQLLLILSHIRHMSN |
| S463P/H524L | | KGMELLYSMKCKNVVPLYDLLLEAADAHRLHAPTSRGGASVEETDQ |
| | | SHLATAGSTSSHSLQKYYITGEAEGFPAT |
| ERT2 mutant | 52 | AGDMRAANLWPSPLMIKRSKKNSLALSLTADQMVSALLDAEPPILYS |
| 41, as included | | EYDPTRPFSEASMMGLLTNLADREIVHMINWAKRVPGFVDLTLHDQV |
| in SB06149 | | HLLECAWLEILMIGVVWRSMEHPVKLLFAPNLVLDRDEGKCVEGMV |
| L354I/L391V/ | | EIFDMLLATSSRFRMMNLQGEEFVCLKSIILLNSGVYTFLSSTLKSLEE |
| L409V/ | | KDHIHRVLDKITDTLIHLMAKAGLTLQQQHQRLAQLLLILSHIRHMSN |
| N413D/ | | KGMELLYSMKCKNVVPLYDLLLEAADAHRLHAPTSRGGASVEETDQ |
| Q414E/H524L | | SHLATAGSTSSHSLQKYYITGEAEGFPAT |
| ERT2 mutant | 53 | AGDMRAANLWPSPLMIKRSKKNSLALSLTADQMVSALLDAEPPILYS |
| 43, as included | | EYDPTRPFSEASMMGLLTNLADRELVHMINWAKRVPGFVDLTLHDQ |
| in SB06150 | | VHLLECAWLEILMIGLVWRSMEHPVKLLFAPNLLLDRNEGKCVEGM |
| Q414E/S463P/ | | VEIFDMLLATSSRFRMMNLQGEEFVCLKSIILLNSGVYTFLPSTLKSLE |
| H524L | | EKDHIHRVLDKITDTLIHLMAKAGLTLQQQHQRLAQLLLILSHIRHMS |
| | | NKGMELLYSMKCKNVVPLYDLLLEAADAHRLHAPTSRGGASVEETD |
| | | QSHLATAGSTSSHSLQKYYITGEAEGFPAT |
| ERT2 mutant | 175 | AGDMRAANLWPSPLMIKRSKKNSLALSLTADQMVSALLDAEPPILYS |
| 46, as included | | EYDPTRPFSEASMMGLLTNLADRELVHMINWAKRVPGFVDLTLHDQ |
| in SB06151 | | VHLLECAWMEILMIGVVWRSMEHPVKLLFAPNLVLDRDQGKCVEGL |
| L384M/ | | VEIFDMLLATSSRFRMMNLQGEEFVCLKSIILLNSGVYTFLPSTLKSLE |
| L391V/L409V/ | | EKDHIHR VLDKITDTLIHLMAKAGLTLQQQHQRLAQLLLILSHIRHAS |
| N413D/ | | NKGMEFLYSMKCKNVVPLYDLLLEAADAHRLHAPTSRGGASVEETD |
| M421L/S463P/ | | QSHLATAGSTSSHSLQKYYITGEAEGFPAT |
| M517A/ | | |
| H524F | | |
| ERT2 mutant | 176 | AGDMRAANLWPSPLMIKRSKKNSLALSLTADQMVSALLDAEPPILYS |
| 40, as included | | EYDPTRPFSEASMMGLLTNLADRELVHMINWAKRVPGFVDLTLHDQ |
| in SB06152 | | VHLLECAWMEILMIGVVWRSMEHPVKLLFAPNLLLDRDQGKCVEGM |
| L384M/ | | VEIFDMLLATSSRFRMMNLQGEEFVCLKSIILLNSGVYTFLSSTLKSLE |
| L391V/ | | EKDHIHRVLDKITDTLIHLMAKAGLTLQQQHQRLAQLLLILSHIRHMS |
| N413D/ | | NKGMEFLYSMKCKNVVPLYDLLLEAADAHRLHAPTSRGGASVEETD |
| H524F | | QSHLATAGSTSSHSLQKYYITGEAEGFPAT |
| 4X ZF5 BD + | 177 | cgggtttcgtaacaatcgcatgaggattcgcaacgcctttGAAGCAGTCGACGCCGAAgtccc |
| YB-TATA min | | gtctcagtaaaggttGAAGCAGTCGACGCCGAAgaatcggactgccttcgtatGAAGCA |
| (Syn promoter) | | GTCGACGCCGAAggtatcagtcgcctcggaatGAAGCAGTCGACGCCGAAgattc |
| | | gtaagaggctcactctcccttacacggagtggataACTAGTTCTAGAGGGTATATAATG |
| | | GGGGCCA |
| IL-12 | 178 | ATGTGCCATCAGCAACTCGTCATCTCCTGGTTCTCCCTTGTGTTCCT |
| | | CGCTTCCCCTCTGGTCGCCATTTGGGAACTGAAGAAGGACGTCTAC |
| | | GTGGTCGAGCTGGATTGGTACCCGGACGCCCCTGGAGAAATGGTC |
| | | GTGCTGACTTGCGATACGCCAGAAGAGGACGGCATAACCTGGACC |
| | | CTGGATCAGAGCTCCGAGGTGCTCGGAAGCGGAAAGACCCTGACC |
| | | ATTCAAGTCAAGGAGTTCGGCGACGCGGGCCAGTACACTTGCCAC |
| | | AAGGGTGGCGAAGTGCTGTCCCACTCCCTGCTGCTGCTGCACAAG |
| | | AAAGAGGATGGAATCTGGTCCACTGACATCCTCAAGGACCAAAAA |
| | | GAACCGAAGAACAAGACCTTCCTCCGCTGCGAAGCCAAGAACTAC |
| | | AGCGGTCGGTTCACCTGTTGGTGGCTGACGACAATCTCCACCGACC |
| | | TGACTTTCTCCGTGAAGTCGTCACGGGGATCAAGCGATCCTCAGGG |
| | | CGTGACCTGTGGAGCCGCCACTCTGTCCGCCGAGAGAGTCAGGGG |
| | | AGACAACAAGGAATATGAGTACTCCGTGGAATGCCAGGAGGACA |
| | | GCGCCTGCCCTGCCGCGGAAGAGTCCCTGCCTATCGAGGTCATGGT |
| | | CGATGCCGTGCATAAGCTGAAATACGAGAACTACACTTCCTCCTTC |
| | | TTTATCCGCGACATCATCAAGCCTGACCCCCCCAAGAACTTGCAGC |
| | | TGAAGCCACTCAAGAACTCCCGCCAAGTGGAAGTGTCTTGGGAAT |
| | | ATCCAGACACTTGGAGCACCCCGCACTCATACTTCTCGCTCACTTT |
| | | CTGTGTGCAAGTGCAGGGAAAGTCCAAACGGGAGAAGAAAGACC |
| | | GGGTGTTCACCGACAAAACCTCCGCCACTGTGATTTGTCGGAAGA |
| | | ACGCGTCAATCAGCGTCCGGGCGCAGGATAGATACTACTCGTCCT |
| | | CCTGGAGCGAATGGGCCAGCGTGCCTTGTTCCGGTGGCGGATCAG |
| | | GCGGAGGTTCAGGAGGAGGCTCCGGAGGAGGTTCCCGGAACCTCC |
| | | CTGTGGCAACCCCCGACCCTGGAATGTTCCCGTGCCTACACCACTC |
| | | CCAAAACCTCCTGAGGGCTGTGTCGAACATGTTGCAGAAGGCCCG |
| | | CCAGACCCTTGAGTTCTACCCCTGCACCTCGGAAGAAATTGATCAC |
| | | GAGGACATCACCAAGGACAAGACCTCGACCGTGGAAGCCTGCCTG |
| | | CCGCTGGAACTGACCAAGAACGAATCGTGTCTGAACTCCCGCGAG |
| | | ACAAGCTTTATCACTAACGGCAGCTGCCTGGCGTCGAGAAAGACC |
| | | TCATTCATGATGGCGCTCTGTCTTTCCTCGATCTACGAAGATCTGA |
| | | AGATGTATCAGGTCGAGTTCAAGACCATGAACGCCAAGCTGCTCA |
| | | TGGACCCGAAGCGGCAGATCTTCCTGGACCAGAATATGCTCGCCG |
| | | TGATTGATGAACTGATGCAGGCCCTGAATTTCAACTCCGAGACTGT |
| | | GCCTCAAAAGTCCAGCCTGGAAGAACCGGACTTCTACAAGACCAA |
| | | GATCAAGCTGTGCATCCTGTTGCACGCTTTCCGCATTCGAGCCGTG |
| | | ACCATTGACCGCGTGATGTCCTACCTGAACGCCAGT |
| IL-12 | 58 | MCHQQLVISWFSLVFLASPLVAIWELKKDVYVVELDWYPDAPGEMV |
| | | VLTCDTPEEDGITWTLDQSSEVLGSGKTLTIQVKEFGDAGQYTCHKG |
| | | GEVLSHSLLLLHKKEDGIWSTDILKDQKEPKNKTFLRCEAKNYSGRFT |
| | | CWWLTTISTDLTFSVKSSRGSSDPQGVTCGAATLSAERVRGDNKEYE |
| | | YSVECQEDSACPAAEESLPIEVMVDAVHKLKYENYTSSFFIRDIIKPDP |
| | | PKNLQLKPLKNSRQVEVSWEYPDTWSTPHSYFSLTFCVQVQGKSKRE |
| | | KKDRVFTDKTSATVICRKNASISVRAQDRYYSSSWSEWASVPCSGGG |
| | | SGGGSGGGSGGGSRNLPVATPDPGMFPCLHHSQNLLRAVSNMLQKA |
| | | RQTLEFYPCTSEEIDHEDITKDKTSTVEACLPLELTKNESCLNSRETSFI |
| | | TNGSCLASRKTSFMMALCLSSIYEDLKMYQVEFKTMNAKLLMDPKR |
| | | QIFLDQNMLA IDELMQALNFNSETVPQKSSLEEPDFYKTKIKLCILLH |
| | | AFRIRAVTIDRVMSYLNAS |
| AU/SLDE | 179 | ATTTATTTATTTATTTATTTAacatcggttccCTGTTTAATATTTAAACAG |
| (destabilization | | |
| domain) | | |
| A2 (insulator) | 180 | AGAGCGAGATTCCGTCTCAAAGAAAAAAAAAGTAATGAAATGAAT |
| | | AAAATGAGTCCTAGAGCCAGTAAATGTCGTAAATGTCTCAGCTAG |
| | | TCAGGTAGTAAAAGGTCTCAACTAGGCAGTGGCAGAGCAGGATTC |
| | | AAATTCAGGGCTGTTGTGATGCCTCCGCAGACTCTGAGCGCCACCT |
| | | GGTGGTAATTTGTCTGTGCCTCTTCTGACGTGGAAGAACAGCAACT |
| | | AACACACTAACACGGCATTTACTATGGGCCAGCCATTGT |
| ZF5-7 DBD | 61 | ATGTCTAGACCTGGCGAGAGGCCCTTCCAGTGCCGGATCTGCATGC |
| (ZF DNA BD) | | GGAACTTCAGCAACATGAGCAACCTGACCAGACACACCCGGACAC |
| | | ACACAGGCGAGAAGCCTTTTCAGTGCAGAATCTGTATGCGCAATTT |
| | | CTCCGACAGAAGCGTGCTGCGGAGACACCTGAGAACCCACACCGG |
| | | CAGCCAGAAACCATTCCAGTGTCGCATCTGTATGAGAAACTTTAGC |
| | | GACCCCTCCAATCTGGCCCGGCACACCAGAACACATACCGGGGAA |
| | | AAACCCTTTCAGTGTAGGATATGCATGAGGAATTTTTCCGACCGGT |
| | | CCAGCCTGAGGCGGCACCTGAGGACACATACTGGCTCCCAAAAGC |
| | | CGTTCCAATGTCGGATATGTATGCGCAACTTTAGCCAGAGCGGCAC |
| | | CCTGCACAGACACACAAGAACCCATACTGGCGAGAAACCTTTCCA |
| | | ATGTAGAATCTGCATGCGAAATTTTTCCCAGCGGCCTAATCTGACC |
| | | AGGCATCTGAGGACCCACCTGAGAGGATCT |
| ZF5-7 DBD | 62 | MSRPGERPFQCRICMRNFSNMSNLTRHTRTHTGEKPFQCRICMRNFSD |
| (ZF DNA BD) | | RSVLRRHLRTHTGSQKPFQCRICMRNFSDPSNLARHTRTHTGEKPFQC |
| | | RICMRNFSDRSSLRRHLRTHTGSQKPFQCRICMRNFSQSGTLHRHTRT |
| | | HTGEKPFQCRICMRNFSQRPNLTRHLRTHLRGS |
| P65 | 63 | GATGAGTTTCCCACCATGGTGTTTCCTTCTGGGCAGATCAGCCAGG |
| (transcriptional | | CCTCGGCCTTGGCCCCGGCCCCTCCCCAAGTCCTGCCCCAGGCTCC |
| activator) | | AGCCCCTGCCCCTGCTCCAGCCATGGTATCAGCTCTGGCCCAGGCC |
| | | CCAGCCCCTGTCCCAGTCCTAGCCCCAGGCCCTCCTCAGGCTGTGG |
| | | CCCCACCTGCCCCCAAGCCCACCCAGGCTGGGGAAGGAACGCTGT |
| | | CAGAGGCCCTGCTGCAGCTGCAGTTTGATGATGAAGACCTGGGGG |
| | | CCTTGCTTGGCAACAGCACAGACCCAGCTGTGTTCACAGACCTGGC |
| | | ATCCGTCGACAACTCCGAGTTTCAGCAGCTGCTGAACCAGGGCAT |
| | | ACCTGTGGCCCCCCACACAACTGAGCCCATGCTGATGGAGTACCC |
| | | TGAGGCTATAACTCGCCTAGTGACAGGGGCCCAGAGGCCCCCCGA |
| | | CCCAGCTCCTGCTCCACTGGGGGCCCCGGGGCTCCCCAATGGCCTC |
| | | CTTTCAGGAGATGAAGACTTCTCCTCCATTGCGGACATGGACTTCT |
| | | CAGCCCTGCTGAGTCAGATCAGCTCC |
| P65 | 64 | DEFPTMVFPSGQISQASALAPAPPQVLPQAPAPAPAPAMVSALAQAPA |
| (transcriptional | | PVPVLAPGPPQAVAPPAPKPTQAGEGTLSEALLQLQFDDEDLGALLG |
| activator) AA | | NSTDPAVFTDLASVDNSEFQQLLNQGIPVAPHTTEPMLMEYPEAITRL |
| | | VTGAQRPPDPAPAPLGAPGLPNGLLSGDEDFSSIADMDFSALLSQISS |
| Mutant 81 | 65 | gctggagacatgagagctgccaacctttggccaagcccgctcatgatcaaacgctctaagaagaacag |
| (ERT mutant) | | cctggccttgtccctgacggccgaccagatggtcagtgccttgttggatgctgagccccccatactct |
| | | attccgagtatgatcctaccagacccttcagtgaagcttcgatgatgggcttactgaccaacctggca |
| | | gacagggagCTGGTTCACATGATCAACTGGGCGAAGAGGGTGCCAGGCTTTGTGGATTT |
| | | GACCCTCCATGATCAGGTCCACCTTCTAGAATGTGCCTGGATGGAG |
| | | ATCCTGATGATTGGTGTGGTCTGGCGCTCCATGGAGCACCCAGTGA |
| | | AGCTACTGTTTGCTCCTAACTTGCTCTTGGACAGGGACCAGGGAAA |
| | | ATGTGTAGAGGGCCTGGTGGAGATCTTCGACATGCTGCTGGCTAC |
| | | ATCATCTCGGTTCCGCATGATGAATCTGCAGGGAGAGGAGTTTGTG |
| | | TGCCTCAAATCTATTATTTTGCTTAATTCTGGAGTGTACACATTTCT |
| | | GCCCAGCACCCTGAAGTCTCTGGAAGAGAAGGACCATATCCACCG |
| | | AGTCCTGGACAAGATCACAGACACTTTGATCCACCTGATGGCCAA |
| | | GGCAGGCCTGACCCTGCAGCAGCAGCACCAGCGGCTGGCCCAGCT |
| | | CCTCCTCATCCTCTCCCACATCAGGCACATGAGTAACAAAGGCATG |
| | | GAGCTGctgtacagcatgaagtgcaagaacgtggtgcccctctatGaCctgctgctggaggcggcgga |
| | | cgcccaccgcctacatgcgcccactagccgtggaggggcatccgtggaggagacggaccaaagccactt |
| | | ggccactgcgggctctacttcatcgcattccttgcaaaagtattacatcacgggggaggcagagggttt |
| | | ccctgccaca |
| Mutant 81 | 66 | AGDMRAANLWPSPLMIKRSKKNSLALSLTADQMVSALLDAEPPILYS |
| (modified | | EYDPTRPFSEASMMGLLTNLADRELVHMINWAKRVPGFVDLTLHDQ |
| ERT2 variant | | VHLLECAWMEILMIGVVWRSMEHPVKLLFAPNLLLDRDQGKCVEGL |
| “mut81”) | | VEIFDMLLATSSRFRMMNLQGEEFVCLKSIILLNSGVYTFLPSTLKSLE |
| | | EKDHIHRVLDKITDTLIHLMAKAGLTLQQQHQRLAQLLLILSHIRHMS |
| | | NKGMELLYSMKCKNVVPLYDLLLEAADAHRLHAPTSRGGASVEETD |
| | | QSHLATAGSTSSHSLQKYYITGEAEGFPAT |
| SB07123 vector | 181 | aagcttgaattcgagcttgcatgcctgcaggtcgttacataacttacggtaaatggcccgcctggctg |
| | | accgcccaacgacccccgcccattgacgtcaataatgacgtatgttcccatagtaacgccaataggga |
| | | ctttccattgacgtcaatgggtggagtatttacggtaaactgcccacttggcagtacatcaagtgtat |
| | | catatgccaagtacgccccctattgacgtcaatgacggtaaatggcccgcctggcattatgcccagta |
| | | catgaccttatgggactttcctacttggcagtacatctacgtattagtcatcgctattaccatggtga |
| | | tgcggttttggcagtacatcaatgggcgtggatagcggtttgactcacggggatttccaagtctccac |
| | | cccattgacgtcaatgggagtttgttttggcaccaaaatcaacgggactttccaaaatgtcgtaacaa |
| | | ctccgccccattgacgcaaatgggcggtaggcgtgtacggtgggaggtctatataagcagagctcaat |
| | | aaaagagcccacaacccctcactcggcgcgccagtcctccgattgactgagtcgcccgggtacccgtg |
| | | tatccaataaaccctcttgcagttgcatccgacttgtggtctcgctgttccttgggagggtctcctct |
| | | gagtgattgactacccgtcagcgggggtctttcatttgggggctcgtccgagatcgggagacccctgc |
| | | ccagggaccaccgacccaccaccgggaggtaagctggccagcaacttatctgtgtctgtccgattgtc |
| | | tagtgtctatgactgattttatgcgcctgcgtcggtactagttagctaactagctctgtatctggcgg |
| | | tacccgtggtggaacgacgagttcggaacacccggccgcaaccctgggagacgtcccagggacttcgg |
| | | gggccgtttttgtggcccgacctgagtcctaaaatcccgatcgtttaggactctttggtgcacccccc |
| | | ttagaggagggatatgtggttctggtaggagacgagaacctaaaacagttcccgcctccgtctgaatt |
| | | tttgctttcggtttgggaccgaagccgcgccgcgcgtcttgtctgctgcagcatcgttctgtgttgtc |
| | | tctgtctgactgtgtttctgtatttgtctgaaaatatgggccccccctcgagtccccagcatgcctgc |
| | | tattctcttcccaatcctcccccttgctgtcctgccccaccccaccccccagaatagaatgacaccta |
| | | ctcagacaatgcgatgcaatttcctcattttattaggaaaggacagtgggagtggcaccttccagggt |
| | | caaggaaggcacgggggaggggcaaacaacagatggctggcaactagaaggcacagttacttaCTGTT |
| | | TAAATATTAAACAGggaaccgatgtTAAATAAATAAATAAATA |
| | | AATGTTTAAACTAGAGTCGCGGCCTCAGTCAGTCACGCATGCCTGC |
| | | AGTttaACTGGCGTTCAGGTAGGACATCACGCGGTCAATGGTCACGG |
| | | CTCGAATGCGGAAAGCGTGCAACAGGATGCACAGCTTGATCTTGG |
| | | TCTTGTAGAAGTCCGGTTCTTCCAGGCTGGACTTTTGAGGCACAGT |
| | | CTCGGAGTTGAAATTCAGGGCCTGCATCAGTTCATCAATCACGGCG |
| | | AGCATATTCTGGTCCAGGAAGATCTGCCGCTTCGGGTCCATGAGCA |
| | | GCTTGGCGTTCATGGTCTTGAACTCGACCTGATACATCTTCAGATC |
| | | TTCGTAGATCGAGGAAAGACAGAGCGCCATCATGAATGAGGTCTT |
| | | TCTCGACGCCAGGCAGCTGCCGTTAGTGATAAAGCTTGTCTCGCGG |
| | | GAGTTCAGACACGATTCGTTCTTGGTCAGTTCCAGCGGCAGGCAG |
| | | GCTTCCACGGTCGAGGTCTTGTCCTTGGTGATGTCCTCGTGATCAA |
| | | TTTCTTCCGAGGTGCAGGGGTAGAACTCAAGGGTCTGGCGGGCCTT |
| | | CTGCAACATGTTCGACACAGCCCTCAGGAGGTTTTGGGAGTGGTGT |
| | | AGGCACGGGAACATTCCAGGGTCGGGGGTTGCCACAGGGAGGTTC |
| | | CGGGAACCTCCTCCGGAGCCTCCTCCTGAACCTCCGCCTGATCCGC |
| | | CACCGGAACAAGGCACGCTGGCCCATTCGCTCCAGGAGGACGAGT |
| | | AGTATCTATCCTGCGCCCGGACGCTGATTGACGCGTTCTTCCGACA |
| | | AATCACAGTGGCGGAGGTTTTGTCGGTGAACACCCGGTCTTTCTTC |
| | | TCCCGTTTGGACTTTCCCTGCACTTGCACACAGAAAGTGAGCGAGA |
| | | AGTATGAGTGCGGGGTGCTCCAAGTGTCTGGATATTCCCAAGACA |
| | | CTTCCACTTGGCGGGAGTTCTTGAGTGGCTTCAGCTGCAAGTTCTT |
| | | GGGGGGGTCAGGCTTGATGATGTCGCGGATAAAGAAGGAGGAAG |
| | | TGTAGTTCTCGTATTTCAGCTTATGCACGGCATCGACCATGACCTC |
| | | GATAGGCAGGGACTCTTCCGCGGCAGGGCAGGCGCTGTCCTCCTG |
| | | GCATTCCACGGAGTACTCATATTCCTTGTTGTCTCCCCTGACTCTCT |
| | | CGGCGGACAGAGTGGCGGCTCCACAGGTCACGCCCTGAGGATCGC |
| | | TTGATCCCCGTGACGACTTCACGGAGAAAGTCAGGTCGGTGGAGA |
| | | TTGTCGTCAGCCACCAACAGGTGAACCGACCGCTGTAGTTCTTGGC |
| | | TTCGCAGCGGAGGAAGGTCTTGTTCTTCGGTTCTTTTTGGTCCTTGA |
| | | GGATGTCAGTGGACCAGATTCCATCCTCTTTCTTGTGCAGCAGCAG |
| | | CAGGGAGTGGGACAGCACTTCGCCACCCTTGTGGCAAGTGTACTG |
| | | GCCCGCGTCGCCGAACTCCTTGACTTGAATGGTCAGGGTCTTTCCG |
| | | CTTCCGAGCACCTCGGAGCTCTGATCCAGGGTCCAGGTTATGCCGT |
| | | CCTCTTCTGGCGTATCGCAAGTCAGCACGACCATTTCTCCAGGGGC |
| | | GTCCGGGTACCAATCCAGCTCGACCACGTAGACGTCCTTCTTCAGT |
| | | TCCCAAATGGCGACCAGAGGGGAAGCGAGGAACACAAGGGAGAA |
| | | CCAGGAGATGACGAGTTGCTGATGGCACATCATGGTGGCGACACC |
| | | GGTACGCGTTGGCCCCCATTATATACCCTCTAGAACTAGTtatccactcc |
| | | gtgtaagggagagtgagcctcttacgaatcTTCGGCGTCGACTGCTTCattccgaggcgactg |
| | | ataccTTCGGCGTCGACTGCTTCatacgaaggcagtccgattcTTCGGCGTCGACT |
| | | GCTTCaacctttactgagacgggacTTCGGCGTCGACTGCTTCaaaggcgttgcgaatcc |
| | | tcatgcgattgttacgaaacccgTTAATTAAAGAGCGAGATTCCGTCTCAAAGA |
| | | AAAAAAAAGTAATGAAATGAATAAAATGAGTCCTAGAGCCAGTA |
| | | AATGTCGTAAATGTCTCAGCTAGTCAGGTAGTAAAAGGTCTCAACT |
| | | AGGCAGTGGCAGAGCAGGATTCAAATTCAGGGCTGTTGTGATGCC |
| | | TCCGCAGACTCTGAGCGCCACCTGGTGGTAATTTGTCTGTGCCTCT |
| | | TCTGACGTGGAAGAACAGCAACTAACACACTAACACGGCATTTAC |
| | | TATGGGCCAGCCATTGTCCATCTAGATGGccgataaaataaaagattttatttagtctc |
| | | cagaaaaaggggggaatgaaagaccccacctgtaggtttggcaagctagctgcagtaacgccattttg |
| | | caaggcatggaaaaataccaaaccaagaatagagaagttcagatcaagggcgggtacatgaaaatagc |
| | | taacgttgggccaaacaggatatctgcggtgagcagtttcggccccggcccggggccaagaacagatg |
| | | gtcaccgcagtttcggccccggcccgaggccaagaacagatggtccccagatatggcccaaccctcag |
| | | cagtttcttaagacccatcagatgtttccaggctcccccaaggacctgaaatgaccctgcgccttatt |
| | | tgaattaaccaatcagcctgcttctcgcttctgttcgcgcgcttctgcttcccgagctctataaaaga |
| | | gctcacaacccctcactcggcgcgccagtcctccgacagactgagtcgcccgggGGATCCGCCACCAT |
| | | GTCTAGACCTGGCGAGAGGCCCTTCCAGTGCCGGATCTGCATGCGGAACTTCAGCAACATG |
| | | AGCAACCTGACCAGACACACCCGGACACACACAGGCGAGAAGCC |
| | | TTTTCAGTGCAGAATCTGTATGCGCAATTTCTCCGACAGAAGCGTG |
| | | CTGCGGAGACACCTGAGAACCCACACCGGCAGCCAGAAACCATTC |
| | | CAGTGTCGCATCTGTATGAGAAACTTTAGCGACCCCTCCAATCTGG |
| | | CCCGGCACACCAGAACACATACCGGGGAAAAACCCTTTCAGTGTA |
| | | GGATATGCATGAGGAATTTTTCCGACCGGTCCAGCCTGAGGCGGC |
| | | ACCTGAGGACACATACTGGCTCCCAAAAGCCGTTCCAATGTCGGA |
| | | TATGTATGCGCAACTTTAGCCAGAGCGGCACCCTGCACAGACACA |
| | | CAAGAACCCATACTGGCGAGAAACCTTTCCAATGTAGAATCTGCA |
| | | TGCGAAATTTTTCCCAGCGGCCTAATCTGACCAGGCATCTGAGGAC |
| | | CCACCTGAGAGGATCTaCCTGCAGGGATGAGTTTCCCACCATGGTG |
| | | TTTCCTTCTGGGCAGATCAGCCAGGCCTCGGCCTTGGCCCCGGCCC |
| | | CTCCCCAAGTCCTGCCCCAGGCTCCAGCCCCTGCCCCTGCTCCAGC |
| | | CATGGTATCAGCTCTGGCCCAGGCCCCAGCCCCTGTCCCAGTCCTA |
| | | GCCCCAGGCCCTCCTCAGGCTGTGGCCCCACCTGCCCCCAAGCCCA |
| | | CCCAGGCTGGGGAAGGAACGCTGTCAGAGGCCCTGCTGCAGCTGC |
| | | AGTTTGATGATGAAGACCTGGGGGCCTTGCTTGGCAACAGCACAG |
| | | ACCCAGCTGTGTTCACAGACCTGGCATCCGTCGACAACTCCGAGTT |
| | | TCAGCAGCTGCTGAACCAGGGCATACCTGTGGCCCCCCACACAAC |
| | | TGAGCCCATGCTGATGGAGTACCCTGAGGCTATAACTCGCCTAGTG |
| | | ACAGGGGCCCAGAGGCCCCCCGACCCAGCTCCTGCTCCACTGGGG |
| | | GCCCCGGGGCTCCCCAATGGCCTCCTTTCAGGAGATGAAGACTTCT |
| | | CCTCCATTGCGGACATGGACTTCTCAGCCCTGCTGAGTCAGATCAG |
| | | CTCCcaattgtgcgtacgcggatcctctgctggagacatgagagctgccaacctttggccaagcccgc |
| | | tcatgatcaaacgctctaagaagaacagcctggccttgtccctgacggccgaccagatggtcagtgcc |
| | | ttgttggatgctgagccccccatactctattccgagtatgatcctaccagacccttcagtgaagcttc |
| | | gatgatgggcttactgaccaacctggcagacagggagCTGGTTCACATGATCAACTGGGCGAAGAGGG |
| | | TGCCAGGCTTTGTGGATTTGACCCTCCATGATCAGGTCCACCTTCTA |
| | | GAATGTGCCTGGATGGAGATCCTGATGATTGGTGTGGTCTGGCGCT |
| | | CCATGGAGCACCCAGTGAAGCTACTGTTTGCTCCTAACTTGCTCTT |
| | | GGACAGGGACCAGGGAAAATGTGTAGAGGGCCTGGTGGAGATCTT |
| | | CGACATGCTGCTGGCTACATCATCTCGGTTCCGCATGATGAATCTG |
| | | CAGGGAGAGGAGTTTGTGTGCCTCAAATCTATTATTTTGCTTAATT |
| | | CTGGAGTGTACACATTTCTGCCCAGCACCCTGAAGTCTCTGGAAGA |
| | | GAAGGACCATATCCACCGAGTCCTGGACAAGATCACAGACACTTT |
| | | GATCCACCTGATGGCCAAGGCAGGCCTGACCCTGCAGCAGCAGCA |
| | | CCAGCGGCTGGCCCAGCTCCTCCTCATCCTCTCCCACATCAGGCAC |
| | | ATGAGTAACAAAGGCATGGAGCTGctgtacagcatgaagtgcaagaacgtggtgccc |
| | | ctctatGaCctgctgctggaggcggcggacgcccaccgcctacatgcgcccactagccgtggaggggc |
| | | atccgtggaggagacggaccaaagccacttggccactgcgggctctacttcatcgcattccttgcaaa |
| | | agtattacatcacgggggaggcagagggtttccctgccacaTaAGTCGACAATCAACCTCtggattac |
| | | aaaatttgtgaaagattgactggtattcttaactatgttgctccttttacgctatgtggatacgctgc |
| | | tttaatgcctttgtatcatgctattgcttcccgtatggctttcattttctcctccttgtataaatcct |
| | | ggttgctgtctctttatgaggagttgtggcccgttgtcaggcaacgtggcgtggtgtgcactgtgttt |
| | | gctgacgcaacccccactggttggggcattgccaccacctgtcagctcctttccgggactttcgcttt |
| | | ccccctccctattgccacggcggaactcatcgccgcctgccttgcccgctgctggacaggggctcggc |
| | | tgttgggcactgacaattccgtggtgttgtcggggaaatcatcgtcctttccttggctgctcgcctgt |
| | | gttgccacctggattctgcgcgggacgtccttctgctacgtcccttcggccctcaatccagcggacc |
| | | ttccttcccgcggcctgctgccggctctgcggcctcttccgcgtctacgccttcgccctcagacgagt |
| | | cggatctccctttgggccgcctccccgcgatatcagtggtccaggctctagttttgactcaacaatat |
| | | caccagctgaagcctatagagtacgagccatagataaaataaaagattttatttagtctccagaaaaa |
| | | ggggggaatgaaagaccccacctgtaggtttggcaagctagcaataaaagagcccacaacccctcact |
| | | ccggggcgccagtctccgattgactgagtcgcccggccgcttcgagcagacatgataagatacattga |
| | | tgagtttggacaaaccacaactagaatgcagtgaaaaaaatgctttatttgtgaaatttgtgatgcta |
| | | ttgctttatttgtaaccattataagctgcaataaacaagttaacaacaacaattgcattcattttatg |
| | | tttcaggttcagggggagatgtgggaggttttttaaagcaagtaaaacctctacaaatgtggtaaaat |
| | | cgataaggatcgggtacccgtgtatccaataaaccctcttgcagttgcatccgacttgtggtctcgct |
| | | gttccttgggagggtctcctctgagtgattgactacccgtcagcgggggtctttcacacatgcagcat |
| | | gtatcaaaattaatttggttttttttcttaagctgtgccttctagttgccagccatctgttgtttgcc |
| | | cctcccccgtgccttccttgaccctggaaggtgccactcccactgtcctttcctaataaaatgaggaa |
| | | attgcatcgcattgtctgagtaggtgtcattctattctggggggggggtggggcaggacagcaagggg |
| | | gaggattgggaagacaatagcaggcatgctggggatgcggtgggctctatggagatcccgcggtacct |
| | | cgcgaatgcatctagatccaatggcctttttggcccagacatgataagatacattgatgagtttggac |
| | | aaaccacaactagaatgcagtgaaaaaaatgctttatttgtgaaatttgtgatgctattgctttattt |
| | | gtaaccattataagctgcaataaacaagttgcggccgcttagccctcccacacataaccagagggcag |
| | | caattcacgaatcccaactgccgtcggctgtccatcactgtccttcactatggctttgatcccaggat |
| | | gcagatcgagaagcacctgtcggcaccgtccgcaggggctcaagatgcccctgttctcatttccgatc |
| | | gcgacgatacaagtcaggttgccagctgccgcagcagcagcagtgcccagcaccacgagttctgcaca |
| | | aggtcccccagtaaaatgatatacattgacaccagtgaagatgcggccgtcgctagagagagctgcgc |
| | | tggcgacgctgtagtcttcagagatggggatgctgttgattgtagccgttgctctttcaatgagggtg |
| | | gattcttcttgagacaaaggcttggccatgcggccgccgctcggtgttcgaggccacacgcgtcac |
| | | cttaatatgcgaagtggacctcggaccgcgccgccccgactgcatctgcgtgttcgaattcgccaatg |
| | | acaagacgctgggggggtttgtgtcatcatagaactaaagacatgcaaatatatttcttccggggggt |
| | | accggcctttttggccATTGGatcggatctggccaaaaaggcccttaagtatttacattaaatggcc |
| | | atagtacttaaagttacattggcttccttgaaataaacatggagtattcagaatgtgtcataaatat |
| | | ttctaattttaagatagtatctccattggctttctactttttcttttatttttttttgtcctctgtct |
| | | tccatttgttgttgttgttgtttgtttgtttgtttgttggttggttggttaatttttttttaaagatc |
| | | ctacactatagttcaagctagactattagctactctgtaacccagggtgaccttgaagtcatgggtag |
| | | cctgctgttttagccttcccacatctaagattacaggtatgagctatcatttttggtatattgattga |
| | | ttgattgattgatgtgtgtgtgtgtgattgtgtttgtgtgtgtgaTtgtgTaTatgtgtgtatggTtg |
| | | tgtgtgaTtgtgtgtatgtatgTTtgtgtgtgaTtgTgtgtgtgtgaTtgtgcatgtgtgtgtgtgtg |
| | | aTtgtgtTtatgtgtatgaTtgtgtgtgtgtgtgtgtgtgtgtgtgtgtgtgtgtgtgtgtgtgtgtt |
| | | gtgTaTaTatatttatggtagtgagagGcaacgctccggctcaggtgtcaggttggtttttgagacag |
| | | agtctttcacttagcttggaattcactggccgtcgttttacaacgtcgtgactgggaaaaccctggcg |
| | | ttacccaacttaatcgccttgcagcacatccccctttcgccagctggcgtaatagcgaagaggcccgc |
| | | accgatcgcccttcccaacagttgcgcagcctgaatggcgaatggcgcctgatgcggtattttctcct |
| | | tacgcatctgtgcggtatttcacaccgcatatggtgcactctcagtacaatctgctctgatgccgcat |
| | | agttaagccagccccgacacccgccaacacccgctgacgcgccctgacgggcttgtctgctcccgg |
| | | catccgcttacagacaagctgtgaccgtctccgggagctgcatgtgtcagaggttttcaccgtcatca |
| | | ccgaaacgcgcgagacgaaagggcctcgtgatacgcctatttttataggttaatgtcatgataataat |
| | | ggtttcttagacgtcaggtggcacttttcggggaaatgtgcgcggaacccctatttgtttatttttct |
| | | aaatacattcaaatatgtatccgctcatgagacaataaccctgataaatgcttcaataatattgaaaa |
| | | aggaagagtatgagtattcaacatttccgtgtcgcccttattcccttttttgcggcattttgccttcc |
| | | tgtttttgctcacccagaaacgctggtgaaagtaaaagatgctgaagatcagttgggtgcacgagtgg |
| | | gttacatcgaactggatctcaacagcggtaagatccttgagagttttcgccccgaagaacgttttcca |
| | | atgatgagcacttttaaagttctgctatgtggcgcggtattatcccgtattgacgccgggcaagagca |
| | | actcggtcgccgcatacactattctcagaatgacttggttgagtactcaccagtcacagaaaagcatc |
| | | ttacggatggcatgacagtaagagaattatgcagtgctgccataaccatgagtgataacactgcggcc |
| | | aacttacttctgacaacgatcggaggaccgaaggagctaaccgcttttttgcacaacatgggggatca |
| | | tgtaactcgccttgatcgttgggaaccggagctgaatgaagccataccaaacgacgagcgtgacacca |
| | | cgatgcctgtagcaatggcaacaacgttgcgcaaactattaactggcgaactacttactctagcttc |
| | | ccggcaacaattaatagactggatggaggcggataaagttgcaggaccacttctgcgctcggcccttc |
| | | cggctggctggtttattgctgataaatctggagccggtgagcgtgggtctcgcggtatcattgcagca |
| | | ctggggccagatggtaagccctcccgtatcgtagttatctacacgacggggagtcaggcaactatgga |
| | | tgaacgaaatagacagatcgctgagataggtgcctcactgattaagcattggtaactgtcagaccaag |
| | | tttactcatatatactttagattgatttaaaacttcatttttaatttaaaaggatctaggtgaagatc |
| | | ctttttgataatctcatgaccaaaatcccttaacgtgagttttcgttccactgagcgtcagaccccgt |
| | | agaaaagatcaaaggatcttcttgagatcctttttttctgcgcgtaatctgctgcttgcaaacaaaaa |
| | | aaccaccgctaccagcggtggtttgtttgccggatcaagagctaccaactctttttccgaaggtaac |
| | | tggcttcagcagagcgcagataccaaatactgtccttctagtgtagccgtagttaggccaccacttca |
| | | agaactctgtagcaccgcctacatacctcgctctgctaatcctgttaccagtggctgctgccagtggc |
| | | gataagtcgtgtcttaccgggttggactcaagacgatagttaccggataaggcgcagcggtcgggctg |
| | | aacggggggttcgtgcacacagcccagcttggagcgaacgacctacaccgaactgagatacctacagc |
| | | gtgagctatgagaaagcgccacgcttcccgaagggagaaaggcggacaggtatccggtaagcggcagg |
| | | gtcggaacaggagagcgcacgagggagcttccagggggaaacgcctggtatctttatagtcctgtcgg |
| | | gtttcgccacctctgacttgagcgtcgatttttgtgatgctcgtcaggggggcggagcctatggaaaa |
| | | acgccagcaacgcggcctttttacggttcctggccttttgctggccttttgctcacatgttctttcc |
| | | tgcgttatcccctgattctgtggataaccgtattaccgcctttgagtgagctgataccgctcgccgca |
| | | gccgaacgaccgagcgcagcgagtcagtgagcgaggaagcggaagagcgcccaatacgcaaaccgcct |
| | | ctccccgcgcgttggccgattcattaatgcagctggcacgacaggtttcccgactggaaagcgggcag |
| | | tgagcgcaacgcaattaatgtgagttagctcactcattaggcaccccaggctttacactttatgcttc |
| | | cggctcgtatgttgtgtggaattgtgagcggataacaatttcacacaggaaacagctatgaccatgat |
| | | tacgcc |
| Mutant 77 | 68 | gctggagacatgagagctgccaacctttggccaagcccgctcatgatcaaacgctctaagaagaacag |
| (ERT mutant) | | cctggccttgtccctgacggccgaccagatggtcagtgccttgttggatgctgagccccccatactct |
| | | attccgagtatgatcctaccagacccttcagtgaagcttcgatgatgggcttactgaccaacctggca |
| | | gacagggagATCGTTCACATGATCAACTGGGCGAAGAGGGTGCCAGGCTTTGTGGATTT |
| | | GACCCTCCATGATCAGGTCCACCTTCTAGAATGTGCCTGGCTAGAG |
| | | ATCCTGATGATTGGTGTGGTCTGGCGCTCCATGGAGCACCCAGTGA |
| | | AGCTACTGTTTGCTCCTAACTTGCTCTTGGACAGGGACCAGGGAAA |
| | | ATGTGTAGAGGGCCTGGTGGAGATCTTCGACATGCTGCTGGCTAC |
| | | ATCATCTCGGTTCCGCATGATGAATCTGCAGGGAGAGGAGTTTGTG |
| | | TGCCTCAAATCTATTATTTTGCTTAATTCTGGAGTGTACACATTTCT |
| | | GCCCAGCACCCTGAAGTCTCTGGAAGAGAAGGACCATATCCACCG |
| | | AGTCCTGGACAAGATCACAGACACTTTGATCCACCTGATGGCCAA |
| | | GGCAGGCCTGACCCTGCAGCAGCAGCACCAGCGGCTGGCCCAGCT |
| | | CCTCCTCATCCTCTCCCACATCAGGCACGCCAGTAACAAAGGCATG |
| | | GAGCTGctgtacagcatgaagtgcaagaacgtggtgcccctctatGaCctgctgctggaggcggcgga |
| | | cgcccaccgcctacatgcgcccactagccgtggaggggcatccgtggaggagacggaccaaagccact |
| | | tggccactgcgggctctacttcatcgcattccttgcaaaagtattacatcacgggggaggcagagggt |
| | | ttccctgccaca |
| Mutant 77 | 172 | AGDMRAANLWPSPLMIKRSKKNSLALSLTADQMVSALLDAEPPILYS |
| (ERT mutant) | | EYDPTRPFSEASMMGLLTNLADREIVHMINWAKRVPGFVDLTLHDQV |
| | | HLLECAWLEILMIGVVWRSMEHPVKLLFAPNLLLDRDQGKCVEGLV |
| | | EIFDMLLATSSRFRMMNLQGEEFVCLKSIILLNSGVYTFLPSTLKSLEE |
| | | KDHIHRVLDKITDTLIHLMAKAGLTLQQQHQRLAQLLLILSHIRHASN |
| | | KGMELLYSMKCKNVVPLYDLLLEAADAHRLHAPTSRGGASVEETDQ |
| | | SHLATAGSTSSHSLQKYYITGEAEGFPAT |
| SB07129 vector | 182 | aagcttgaattcgagcttgcatgcctgcaggtcgttacataacttacggtaaatggcccgcctggctg |
| | | accgcccaacgacccccgcccattgacgtcaataatgacgtatgttcccatagtaacgccaataggga |
| | | ctttccattgacgtcaatgggtggagtatttacggtaaactgcccacttggcagtacatcaagtgtat |
| | | catatgccaagtacgccccctattgacgtcaatgacggtaaatggcccgcctggcattatgcccagta |
| | | catgaccttatgggactttcctacttggcagtacatctacgtattagtcatcgctattaccatggtga |
| | | tgcggttttggcagtacatcaatgggcgtggatagcggtttgactcacggggatttccaagtctccac |
| | | cccattgacgtcaatgggagtttgttttggcaccaaaatcaacgggactttccaaaatgtcgtaaca |
| | | actccgccccattgacgcaaatgggcggtaggcgtgtacggtgggaggtctatataagcagagctcaa |
| | | taaaagagcccacaacccctcactcggcgcgccagtcctccgattgactgagtcgcccgggtacccgt |
| | | gtatccaataaaccctcttgcagttgcatccgacttgtggtctcgctgttccttgggagggtctcctc |
| | | tgagtgattgactacccgtcagcgggggtctttcatttgggggctcgtccgagatcgggagacccctg |
| | | cccagggaccaccgacccaccaccgggaggtaagctggccagcaacttatctgtgtctgtccgattgt |
| | | ctagtgtctatgactgattttatgcgcctgcgtcggtactagttagctaactagctctgtatctggcg |
| | | tgacgagttcggaacacccggccgcaaccctgggagacgtcccagggacttcgggggccgtttttgtg |
| | | gcgacccgtggtggaacccgacctgagtcctaaaatcccgatcgtttaggactctttggtgcaccccc |
| | | cttagaggagggatatgtggttctggtaggagacgagaacctaaaacagttcccgcctccgtctgaat |
| | | ttttgctttcggtttgggaccgaagccgcgccgcgcgtcttgtctgctgcagcatcgttctgtgttg |
| | | tctctgtctgactgtgtttctgtatttgtctgaaaatatgggccccccctcgagtccccagcatgcc |
| | | tgctattctcttcccaatcctcccccttgctgtcctgccccaccccaccccccagaatagaatgacac |
| | | ctactcagacaatgcgatgcaatttcctcattttattaggaaaggacagtgggagtggcaccttccag |
| | | ggtcaaggaaggcacgggggaggggcaaacaacagatggctggcaactagaaggcacagttac |
| | | ttaCTGTTTAAATATTAAACAGggaaccgatgtTAAATAAATAAATAAATA |
| | | AATGTTTAAACTAGAGTCGCGGCCTCAGTCAGTCACGCATGCCTGC |
| | | AGTttaACTGGCGTTCAGGTAGGACATCACGCGGTCAATGGTCACGG |
| | | CTCGAATGCGGAAAGCGTGCAACAGGATGCACAGCTTGATCTTGG |
| | | TCTTGTAGAAGTCCGGTTCTTCCAGGCTGGACTTTTGAGGCACAGT |
| | | CTCGGAGTTGAAATTCAGGGCCTGCATCAGTTCATCAATCACGGCG |
| | | AGCATATTCTGGTCCAGGAAGATCTGCCGCTTCGGGTCCATGAGCA |
| | | GCTTGGCGTTCATGGTCTTGAACTCGACCTGATACATCTTCAGATC |
| | | TTCGTAGATCGAGGAAAGACAGAGCGCCATCATGAATGAGGTCTT |
| | | TCTCGACGCCAGGCAGCTGCCGTTAGTGATAAAGCTTGTCTCGCGG |
| | | GAGTTCAGACACGATTCGTTCTTGGTCAGTTCCAGCGGCAGGCAG |
| | | GCTTCCACGGTCGAGGTCTTGTCCTTGGTGATGTCCTCGTGATCAA |
| | | TTTCTTCCGAGGTGCAGGGGTAGAACTCAAGGGTCTGGCGGGCCTT |
| | | CTGCAACATGTTCGACACAGCCCTCAGGAGGTTTTGGGAGTGGTGT |
| | | AGGCACGGGAACATTCCAGGGTCGGGGGTTGCCACAGGGAGGTTC |
| | | CGGGAACCTCCTCCGGAGCCTCCTCCTGAACCTCCGCCTGATCCGC |
| | | CACCGGAACAAGGCACGCTGGCCCATTCGCTCCAGGAGGACGAGT |
| | | AGTATCTATCCTGCGCCCGGACGCTGATTGACGCGTTCTTCCGACA |
| | | AATCACAGTGGCGGAGGTTTTGTCGGTGAACACCCGGTCTTTCTTC |
| | | TCCCGTTTGGACTTTCCCTGCACTTGCACACAGAAAGTGAGCGAGA |
| | | AGTATGAGTGCGGGGTGCTCCAAGTGTCTGGATATTCCCAAGACA |
| | | CTTCCACTTGGCGGGAGTTCTTGAGTGGCTTCAGCTGCAAGTTCTT |
| | | GGGGGGGTCAGGCTTGATGATGTCGCGGATAAAGAAGGAGGAAG |
| | | TGTAGTTCTCGTATTTCAGCTTATGCACGGCATCGACCATGACCTC |
| | | GATAGGCAGGGACTCTTCCGCGGCAGGGCAGGCGCTGTCCTCCTG |
| | | GCATTCCACGGAGTACTCATATTCCTTGTTGTCTCCCCTGACTCTCT |
| | | CGGCGGACAGAGTGGCGGCTCCACAGGTCACGCCCTGAGGATCGC |
| | | TTGATCCCCGTGACGACTTCACGGAGAAAGTCAGGTCGGTGGAGA |
| | | TTGTCGTCAGCCACCAACAGGTGAACCGACCGCTGTAGTTCTTGGC |
| | | TTCGCAGCGGAGGAAGGTCTTGTTCTTCGGTTCTTTTTGGTCCTTGA |
| | | GGATGTCAGTGGACCAGATTCCATCCTCTTTCTTGTGCAGCAGCAG |
| | | CAGGGAGTGGGACAGCACTTCGCCACCCTTGTGGCAAGTGTACTG |
| | | GCCCGCGTCGCCGAACTCCTTGACTTGAATGGTCAGGGTCTTTCCG |
| | | CTTCCGAGCACCTCGGAGCTCTGATCCAGGGTCCAGGTTATGCCGT |
| | | CCTCTTCTGGCGTATCGCAAGTCAGCACGACCATTTCTCCAGGGGC |
| | | GTCCGGGTACCAATCCAGCTCGACCACGTAGACGTCCTTCTTCAGT |
| | | TCCCAAATGGCGACCAGAGGGGAAGCGAGGAACACAAGGGAGAA |
| | | CCAGGAGATGACGAGTTGCTGATGGCACATCATGGTGGCGACACC |
| | | GGTACGCGTTGGCCCCCATTATATACCCTCTAGAACTAGTtatccactcc |
| | | gtgtaagggagagtgagcctcttacgaatcTTCGGCGTCGACTGCTTCattccgaggcgactg |
| | | ataccTTCGGCGTCGACTGCTTCatacgaaggcagtccgattcTTCGGCGTCGACT |
| | | GCTTCaacctttactgagacgggacTTCGGCGTCGACTGCTTCaaaggcgttgcgaatcc |
| | | tcatgcgattgttacgaaacccgTTAATTAAAGAGCGAGATTCCGTCTCAAAGA |
| | | AAAAAAAAGTAATGAAATGAATAAAATGAGTCCTAGAGCCAGTA |
| | | AATGTCGTAAATGTCTCAGCTAGTCAGGTAGTAAAAGGTCTCAACT |
| | | AGGCAGTGGCAGAGCAGGATTCAAATTCAGGGCTGTTGTGATGCC |
| | | TCCGCAGACTCTGAGCGCCACCTGGTGGTAATTTGTCTGTGCCTCT |
| | | TCTGACGTGGAAGAACAGCAACTAACACACTAACACGGCATTTAC |
| | | TATGGGCCAGCCATTGTCCATCTAGATGGccgataaaataaaagattttatttagtctc |
| | | cagaaaaaggggggaatgaaagaccccacctgtaggtttggcaagctagctgcagtaacgccattttg |
| | | caaggcatggaaaaataccaaaccaagaatagagaagttcagatcaaggggggtacatgaaaatagct |
| | | aacgttgggccaaacaggatatctgcggtgagcagtttcggccccggcccggggccaagaacagatg |
| | | gtcaccgcagtttcggccccggcccgaggccaagaacagatggtccccagatatggcccaaccctca |
| | | gcagtttcttaagacccatcagatgtttccaggctcccccaaggacctgaaatgaccctgcgcctt |
| | | atttgaattaaccaatcagcctgcttctcgcttctgttcgcgcgcttctgcttcccgagctctataa |
| | | aagagctcacaacccctcactcggcgcgccagtcctccgacagactgagtcgcccgggGGATCCGCC |
| | | ACCATGTCTAGACCTGGCGAGAGGCCCTTCCAGTGCCGGATCTGCATGCGGAACTTCAGCAACATG |
| | | AGCAACCTGACCAGACACACCCGGACACACACAGGCGAGAAGCC |
| | | TTTTCAGTGCAGAATCTGTATGCGCAATTTCTCCGACAGAAGCGTG |
| | | CTGCGGAGACACCTGAGAACCCACACCGGCAGCCAGAAACCATTC |
| | | CAGTGTCGCATCTGTATGAGAAACTTTAGCGACCCCTCCAATCTGG |
| | | CCCGGCACACCAGAACACATACCGGGGAAAAACCCTTTCAGTGTA |
| | | GGATATGCATGAGGAATTTTTCCGACCGGTCCAGCCTGAGGCGGC |
| | | ACCTGAGGACACATACTGGCTCCCAAAAGCCGTTCCAATGTCGGA |
| | | TATGTATGCGCAACTTTAGCCAGAGCGGCACCCTGCACAGACACA |
| | | CAAGAACCCATACTGGCGAGAAACCTTTCCAATGTAGAATCTGCA |
| | | TGCGAAATTTTTCCCAGCGGCCTAATCTGACCAGGCATCTGAGGAC |
| | | CCACCTGAGAGGATCTaCCTGCAGGGATGAGTTTCCCACCATGGTG |
| | | TTTCCTTCTGGGCAGATCAGCCAGGCCTCGGCCTTGGCCCCGGCCC |
| | | CTCCCCAAGTCCTGCCCCAGGCTCCAGCCCCTGCCCCTGCTCCAGC |
| | | CATGGTATCAGCTCTGGCCCAGGCCCCAGCCCCTGTCCCAGTCCTA |
| | | GCCCCAGGCCCTCCTCAGGCTGTGGCCCCACCTGCCCCCAAGCCCA |
| | | CCCAGGCTGGGGAAGGAACGCTGTCAGAGGCCCTGCTGCAGCTGC |
| | | AGTTTGATGATGAAGACCTGGGGGCCTTGCTTGGCAACAGCACAG |
| | | ACCCAGCTGTGTTCACAGACCTGGCATCCGTCGACAACTCCGAGTT |
| | | TCAGCAGCTGCTGAACCAGGGCATACCTGTGGCCCCCCACACAAC |
| | | TGAGCCCATGCTGATGGAGTACCCTGAGGCTATAACTCGCCTAGTG |
| | | ACAGGGGCCCAGAGGCCCCCCGACCCAGCTCCTGCTCCACTGGGG |
| | | GCCCCGGGGCTCCCCAATGGCCTCCTTTCAGGAGATGAAGACTTCT |
| | | CCTCCATTGCGGACATGGACTTCTCAGCCCTGCTGAGTCAGATCAG |
| | | CTCCcaattgtgcgtacgcggatcctctgctggagacatgagagctgccaacctttggccaagcccgc |
| | | tcatgatcaaacgctctaagaagaacagcctggccttgtccctgacggccgaccagatggtcagtgc |
| | | cttgttggatgctgagccccccatactctattccgagtatgatcctaccagacccttcagtgaagctt |
| | | cgatgatgggcttactgaccaacctggcagacagggagATCGTTCACATGATCAACTGGGCGAAGAGGGT |
| | | GCCAGGCTTTGTGGATTTGACCCTCCATGATCAGGTCCACCTTCTA |
| | | GAATGTGCCTGGCTAGAGATCCTGATGATTGGTGTGGTCTGGCGCT |
| | | CCATGGAGCACCCAGTGAAGCTACTGTTTGCTCCTAACTTGCTCTT |
| | | GGACAGGGACCAGGGAAAATGTGTAGAGGGCCTGGTGGAGATCTT |
| | | CGACATGCTGCTGGCTACATCATCTCGGTTCCGCATGATGAATCTG |
| | | CAGGGAGAGGAGTTTGTGTGCCTCAAATCTATTATTTTGCTTAATT |
| | | CTGGAGTGTACACATTTCTGCCCAGCACCCTGAAGTCTCTGGAAGA |
| | | GAAGGACCATATCCACCGAGTCCTGGACAAGATCACAGACACTTT |
| | | GATCCACCTGATGGCCAAGGCAGGCCTGACCCTGCAGCAGCAGCA |
| | | CCAGCGGCTGGCCCAGCTCCTCCTCATCCTCTCCCACATCAGGCAC |
| | | GCCAGTAACAAAGGCATGGAGCTGctgtacagcatgaagtgcaagaacgtggtgccc |
| | | ctctatGaCctgctgctggaggcggcggacgcccaccgcctacatgcgcccactagccgtggaggggc |
| | | atccgtggaggagacggaccaaagccacttggccactgcgggctctacttcatcgcattccttgcaa |
| | | aagtattacatcacgggggaggcagagggtttccctgccacaTaAGTCGACAATCAACCTCtggatta |
| | | caaaatttgtgaaagattgactggtattcttaactatgttgctccttttacgctatgtggatacgctg |
| | | ctttaatgcctttgtatcatgctattgcttcccgtatggctttcattttctcctccttgtataaatc |
| | | ctggttgctgtctctttatgaggagttgtggcccgttgtcaggcaacgtggcgtggtgtgcactgtgt |
| | | ttgctgacgcaacccccactggttggggcattgccaccacctgtcagctcctttccgggactttcgct |
| | | ttccccctccctattgccacggcggaactcatcgccgcctgccttgcccgctgctggacaggggctcg |
| | | gctgttgggcactgacaattccgtggtgttgtcggggaaatcatcgtcctttccttggctgctcgcct |
| | | gtgttgccacctggattctgcgcgggacgtccttctgctacgtcccttcggccctcaatccagcggac |
| | | cttccttcccgcggcctgctgccggctctgcggcctcttccgcgtctacgccttcgccctcagacgag |
| | | tcggatctccctttgggccgcctccccgcgatatcagtggtccaggctctagttttgactcaacaata |
| | | tcaccagctgaagcctatagagtacgagccatagataaaataaaagattttatttagtctccagaaaa |
| | | aggggggaatgaaagaccccacctgtaggtttggcaagctagcaataaaagagcccacaacccctcac |
| | | tcggggcgccagtcctccgattgactgagtcgcccggccgcttcgagcagacatgataagatacattg |
| | | atgagtttggacaaaccacaactagaatgcagtgaaaaaaatgctttatttgtgaaatttgtgatgct |
| | | attgctttatttgtaaccattataagctgcaataaacaagttaacaacaacaattgcattcattttat |
| | | gtttcaggttcagggggagatgtgggaggttttttaaagcaagtaaaacctctacaaatgtggtaaaa |
| | | tcgataaggatcgggtacccgtgtatccaataaaccctcttgcagttgcatccgacttgtggtctcgc |
| | | tgttccttgggagggtctcctctgagtgattgactacccgtcagcgggggtctttcacacatgcagca |
| | | tgtatcaaaattaatttggttttttttcttaagctgtgccttctagttgccagccatctgttgtttgc |
| | | ccctcccccgtgccttccttgaccctggaaggtgccactcccactgtcctttcctaataaaatgagg |
| | | aaattgcatcgcattgtctgagtaggtgtcattctattctggggggtggggggggcaggacagcaagg |
| | | gggaggattgggaagacaatagcaggcatgctggggatgcggtgggctctatggagatcccgcggtac |
| | | ctcgcgaatgcatctagatccaatggcctttttggcccagacatgataagatacattgatgagtttgg |
| | | acaaaccacaactagaatgcagtgaaaaaaatgctttatttgtgaaatttgtgatgctattgctttat |
| | | ttgtaaccattataagctgcaataaacaagttgcggccgcttagccctcccacacataaccagagg |
| | | gcagcaattcacgaatcccaactgccgtcggctgtccatcactgtccttcactatggctttgatccca |
| | | ggatgcagatcgagaagcacctgtcggcaccgtccgcaggggctcaagatgcccctgttctcatttc |
| | | cgatcgcgacgatacaagtcaggttgccagctgccgcagcagcagcagtgcccagcaccacgagttct |
| | | gcacaaggtcccccagtaaaatgatatacattgacaccagtgaagatgcggccgtcgctagagagag |
| | | ctgcgctggcgacgctgtagtcttcagagatggggatgctgttgattgtagccgttgctctttcaat |
| | | gagggtggattcttcttgagacaaaggcttggccatgcggccgccgctcggtgttcgaggccacacg |
| | | cgtcaccttaatatgcgaagtggacctcggaccgcgccgccccgactgcatctgcgtgttcgaattcg |
| | | ccaatgacaagacgctgggcggggtttgtgtcatcatagaactaaagacatgcaaatatatttcttcc |
| | | ggggggtaccggcctttttggccATTGGatcggatctggccaaaaaggcccttaagtatttacattaa |
| | | atggccatagtacttaaagttacattggcttccttgaaataaacatggagtattcagaatgtgtcata |
| | | aatatttctaattttaagatagtatctccattggctttctactttttcttttatttttttttgtcctc |
| | | tgtcttccatttgttgttgttgttgtttgtttgtttgtttgttggttggttggttaatttttttttaa |
| | | agatcctacactatagttcaagctagactattagctactctgtaacccagggtgaccttgaagtcatg |
| | | ggtagcctgctgttttagccttcccacatctaagattacaggtatgagctatcatttttggtatattg |
| | | attgattgattgattgatgtgtgtgtgtgtgattgtgtttgtgtgtgtgaTtgtgTaTatgtgtgta |
| | | tggTtgtgtgtgaTtgtgtgtatgtatgTTtgtgtgtgaTtgTgtgtgtgtgaTtgtgcatgtgtgtg |
| | | tgtgtgaTtgtgtTtatgtgtatgaTtgtgtgtgtgtgtgtgtgtgtgtgtgtgtgtgtgtgtgtgtg |
| | | tgtgttgtgTaTaTatatttatggtagtgagagGcaacgctccggctcaggtgtcaggttggtttttg |
| | | agacagagtctttcacttagcttggaattcactggccgtcgttttacaacgtcgtgactgggaaaacc |
| | | ctggcgttacccaacttaatcgccttgcagcacatccccctttcgccagctggcgtaatagcgaagag |
| | | gcccgcaccgatcgcccttcccaacagttgcgcagcctgaatggcgaatggcgcctgatgcggtattt |
| | | tctccttacgcatctgtgcggtatttcacaccgcatatggtgcactctcagtacaatctgctctgatg |
| | | ccgcatagttaagccagccccgacacccgccaacacccgctgacgcgccctgacgggcttgtctgctc |
| | | ccggcatccgcttacagacaagctgtgaccgtctccgggagctgcatgtgtcagaggttttcaccgt |
| | | catcaccgaaacgcgcgagacgaaagggcctcgtgatacgcctatttttataggttaatgtcatgat |
| | | aataatggtttcttagacgtcaggtggcacttttcggggaaatgtgcgcggaacccctatttgttta |
| | | tttttctaaatacattcaaatatgtatccgctcatgagacaataaccctgataaatgcttcaataata |
| | | ttgaaaaaggaagagtatgagtattcaacatttccgtgtcgcccttattcccttttttgcggcatttt |
| | | gccttcctgtttttgctcacccagaaacgctggtgaaagtaaaagatgctgaagatcagttgggtgca |
| | | cgagtgggttacatcgaactggatctcaacagcggtaagatccttgagagttttcgccccgaagaacg |
| | | ttttccaatgatgagcacttttaaagttctgctatgtggcgcggtattatcccgtattgacgccgg |
| | | gcaagagcaactcggtcgccgcatacactattctcagaatgacttggttgagtactcaccagtcaca |
| | | gaaaagcatcttacggatggcatgacagtaagagaattatgcagtgctgccataaccatgagtgata |
| | | acactgcggccaacttacttctgacaacgatcggaggaccgaaggagctaaccgcttttttgcacaa |
| | | catgggggatcatgtaactcgccttgatcgttgggaaccggagctgaatgaagccataccaaacgacg |
| | | agcgtgacaccacgatgcctgtagcaatggcaacaacgttgcgcaaactattaactggcgaactactt |
| | | actctagcttcccggcaacaattaatagactggatggaggcggataaagttgcaggaccacttctgcg |
| | | ctcggcccttccggctggctggtttattgctgataaatctggagccggtgagcgtgggtctcgcggta |
| | | tcattgcagcactggggccagatggtaagccctcccgtatcgtagttatctacacgacggggagtcag |
| | | gcaactatggatgaacgaaatagacagatcgctgagataggtgcctcactgattaagcattggtaact |
| | | gtcagaccaagtttactcatatatactttagattgatttaaaacttcatttttaatttaaaaggatct |
| | | aggtgaagatcctttttgataatctcatgaccaaaatcccttaacgtgagttttcgttccactgagcg |
| | | tcagaccccgtagaaaagatcaaaggatcttcttgagatcctttttttctgcgcgtaatctgctgct |
| | | tgcaaacaaaaaaaccaccgctaccagcggtggtttgtttgccggatcaagagctaccaactcttttt |
| | | ccgaaggtaactggcttcagcagagcgcagataccaaatactgtccttctagtgtagccgtagttag |
| | | gccaccacttcaagaactctgtagcaccgcctacatacctcgctctgctaatcctgttaccagtggct |
| | | gctgccagtggcgataagtcgtgtcttaccgggttggactcaagacgatagttaccggataaggcgc |
| | | agcggtcgggctgaacggggggttcgtgcacacagcccagcttggagcgaacgacctacaccgaact |
| | | gagatacctacagcgtgagctatgagaaagcgccacgcttcccgaagggagaaaggcggacaggtatc |
| | | cggtaagcggcagggtcggaacaggagagcgcacgagggagcttccagggggaaacgcctggtatct |
| | | ttatagtcctgtcgggtttcgccacctctgacttgagcgtcgatttttgtgatgctcgtcagggggg |
| | | cggagcctatggaaaaacgccagcaacgcggcctttttacggttcctggccttttgctggcc |
| | | ttttgctcacatgttctttcctgcgttatcccctgattctgtggataaccgtattaccgcctttgagt |
| | | gagctgataccgctcgccgcagccgaacgaccgagcgcagcgagtcagtgagcgaggaagcggaagag |
| | | cgcccaatacgcaaaccgcctctccccgcgcgttggccgattcattaatgcagctggcacgacaggt |
| | | ttcccgactggaaagcgggcagtgagcgcaacgcaattaatgtgagttagctcactcattaggcaccc |
| | | caggctttacactttatgcttccggctcgtatgttgtgtggaattgtgagcggataacaatttcaca |
| | | caggaaacagctatgaccatgattacgcc |
| Mutant 62 | 71 | gctggagacatgagagctgccaacctttggccaagcccgctcatgatcaaacgctctaagaagaacag |
| (ERT mutant) | | cctggccttgtccctgacggccgaccagatggtcagtgccttgttggatgctgagccccccatactc |
| | | tattccgagtatgatcctaccagacccttcagtgaagcttcgatgatgggcttactgaccaacctgg |
| | | cagacagggagCTGGTTCACATGATCAACTGGGCGAAGAGGGTGCCAGGCTTTGTGGATTT |
| | | GACCCTCCATGATCAGGTCCACCTTCTAGAATGTGCCTGGCTAGAG |
| | | ATCCTGATGATTGGTCTCGTCTGGCGCTCCATGGAGCACCCAGTGA |
| | | AGCTACTGTTTGCTCCTAACTTGGTGTTGGACAGGGACGAGGGAA |
| | | AATGTGTAGAGGGCCTGGTGGAGATCTTCGACATGCTGCTGGCTA |
| | | CATCATCTCGGTTCCGCATGATGAATCTGCAGGGAGAGGAGTTTGT |
| | | GTGCCTCAAATCTATTATTTTGCTTAATTCTGGAGTGTACACATTTC |
| | | TGCCCAGCACCCTGAAGTCTCTGGAAGAGAAGGACCATATCCACC |
| | | GAGTCCTGGACAAGATCACAGACACTTTGATCCACCTGATGGCCA |
| | | AGGCAGGCCTGACCCTGCAGCAGCAGCACCAGCGGCTGGCCCAGC |
| | | TCCTCCTCATCCTCTCCCACATCAGGCACATGAGTAACAAAGGCAT |
| | | GGAGCTGctgtacagcatgaagtgcaagaacgtggtgcccctctatGaCctgctgctggaggcggcg |
| | | gacgcccaccgcctacatgcgcccactagccgtggaggggcatccgtggaggagacggaccaaagcca |
| | | cttggccactgcgggctctacttcatcgcattccttgcaaaagtattacatcacgggggaggcagag |
| | | ggtttccctgccaca |
| Mutant 62 | 72 | AGDMRAANLWPSPLMIKRSKKNSLALSLTADQMVSALLDAEPPILYS |
| (ERT mutant) | | EYDPTRPFSEASMMGLLTNLADRELVHMINWAKRVPGFVDLTLHDQ |
| | | VHLLECAWLEILMIGLVWRSMEHPVKLLFAPNLVLDRDEGKCVEGL |
| | | VEIFDMLLATSSRFRMMNLQGEEFVCLKSIILLNSGVYTFLPSTLKSLE |
| | | EKDHIHRVLDKITDTLIHLMAKAGLTLQQQHQRLAQLLLILSHIRHMS |
| | | NKGMELLYSMKCKNVVPLYDLLLEAADAHRLHAPTSRGGASVEETD |
| | | QSHLATAGSTSSHSLQKYYITGEAEGFPAT |
| SB07135 vector | 183 | aagcttgaattcgagcttgcatgcctgcaggtcgttacataacttacggtaaatggcccgcctggctg |
| | | accgcccaacgacccccgcccattgacgtcaataatgacgtatgttcccatagtaacgccaataggga |
| | | ctttccattgacgtcaatgggtggagtatttacggtaaactgcccacttggcagtacatcaagtgt |
| | | atcatatgccaagtacgccccctattgacgtcaatgacggtaaatggcccgcctggcattatgcccag |
| | | tacatgaccttatgggactttcctacttggcagtacatctacgtattagtcatcgctattaccatgg |
| | | tgatgcggttttggcagtacatcaatgggcgtggatagcggtttgactcacggggatttccaagtctc |
| | | caccccattgacgtcaatgggagtttgttttggcaccaaaatcaacgggactttccaaaatgtcgta |
| | | acaactccgccccattgacgcaaatgggggtaggcgtgtacggtgggaggtctatataagcagagct |
| | | caataaaagagcccacaacccctcactcggcgcgccagtcctccgattgactgagtcgcccgggtacc |
| | | cgtgtatccaataaaccctcttgcagttgcatccgacttgtggtctcgctgttccttgggagggtc |
| | | tcctctgagtgattgactacccgtcagcgggggtctttcatttgggggctcgtccgagatcgggaga |
| | | cccctgcccagggaccaccgacccaccaccgggaggtaagctggccagcaacttatctgtgtctgt |
| | | ccgattgtctagtgtctatgactgattttatgcgcctgcgtcggtactagttagctaactagctctg |
| | | tatctggcggacccgtggtggaactgacgagttcggaacacccggccgcaaccctgggagacgtccca |
| | | gggacttcgggggccgtttttgtggcccgacctgagtcctaaaatcccgatcgtttaggactctttgg |
| | | tgcaccccccttagaggagggatatgtggttctggtaggagacgagaacctaaaacagttcccgcctc |
| | | cgtctgaatttttgctttcggtttgggaccgaagccgcgccgcgcgtcttgtctgctgcagcatcgtt |
| | | ctgtgttgtctctgtctgactgtgtttctgtatttgtctgaaaatatgggccccccctcgagtcccca |
| | | gcatgcctgctattctcttcccaatcctcccccttgctgtcctgccccaccccaccccccagaataga |
| | | atgacacctactcagacaatgcgatgcaatttcctcattttattaggaaaggacagtgggagtggcac |
| | | cttccagggtcaaggaaggcacgggggaggggcaaacaacagatggctggcaactagaaggcacagtta |
| | | cttaCTGTTTAAATATTAAACAGggaaccgatgtTAAATAAATAAATAAATA |
| | | AATGTTTAAACTAGAGTCGCGGCCTCAGTCAGTCACGCATGCCTGC |
| | | AGTttaACTGGCGTTCAGGTAGGACATCACGCGGTCAATGGTCACGG |
| | | CTCGAATGCGGAAAGCGTGCAACAGGATGCACAGCTTGATCTTGG |
| | | TCTTGTAGAAGTCCGGTTCTTCCAGGCTGGACTTTTGAGGCACAGT |
| | | CTCGGAGTTGAAATTCAGGGCCTGCATCAGTTCATCAATCACGGCG |
| | | AGCATATTCTGGTCCAGGAAGATCTGCCGCTTCGGGTCCATGAGCA |
| | | GCTTGGCGTTCATGGTCTTGAACTCGACCTGATACATCTTCAGATC |
| | | TTCGTAGATCGAGGAAAGACAGAGCGCCATCATGAATGAGGTCTT |
| | | TCTCGACGCCAGGCAGCTGCCGTTAGTGATAAAGCTTGTCTCGCGG |
| | | GAGTTCAGACACGATTCGTTCTTGGTCAGTTCCAGCGGCAGGCAG |
| | | GCTTCCACGGTCGAGGTCTTGTCCTTGGTGATGTCCTCGTGATCAA |
| | | TTTCTTCCGAGGTGCAGGGGTAGAACTCAAGGGTCTGGCGGGCCTT |
| | | CTGCAACATGTTCGACACAGCCCTCAGGAGGTTTTGGGAGTGGTGT |
| | | AGGCACGGGAACATTCCAGGGTCGGGGGTTGCCACAGGGAGGTTC |
| | | CGGGAACCTCCTCCGGAGCCTCCTCCTGAACCTCCGCCTGATCCGC |
| | | CACCGGAACAAGGCACGCTGGCCCATTCGCTCCAGGAGGACGAGT |
| | | AGTATCTATCCTGCGCCCGGACGCTGATTGACGCGTTCTTCCGACA |
| | | AATCACAGTGGCGGAGGTTTTGTCGGTGAACACCCGGTCTTTCTTC |
| | | TCCCGTTTGGACTTTCCCTGCACTTGCACACAGAAAGTGAGCGAGA |
| | | AGTATGAGTGCGGGGTGCTCCAAGTGTCTGGATATTCCCAAGACA |
| | | CTTCCACTTGGCGGGAGTTCTTGAGTGGCTTCAGCTGCAAGTTCTT |
| | | GGGGGGGTCAGGCTTGATGATGTCGCGGATAAAGAAGGAGGAAG |
| | | TGTAGTTCTCGTATTTCAGCTTATGCACGGCATCGACCATGACCTC |
| | | GATAGGCAGGGACTCTTCCGCGGCAGGGCAGGCGCTGTCCTCCTG |
| | | GCATTCCACGGAGTACTCATATTCCTTGTTGTCTCCCCTGACTCTCT |
| | | CGGCGGACAGAGTGGCGGCTCCACAGGTCACGCCCTGAGGATCGC |
| | | TTGATCCCCGTGACGACTTCACGGAGAAAGTCAGGTCGGTGGAGA |
| | | TTGTCGTCAGCCACCAACAGGTGAACCGACCGCTGTAGTTCTTGGC |
| | | TTCGCAGCGGAGGAAGGTCTTGTTCTTCGGTTCTTTTTGGTCCTTGA |
| | | GGATGTCAGTGGACCAGATTCCATCCTCTTTCTTGTGCAGCAGCAG |
| | | CAGGGAGTGGGACAGCACTTCGCCACCCTTGTGGCAAGTGTACTG |
| | | GCCCGCGTCGCCGAACTCCTTGACTTGAATGGTCAGGGTCTTTCCG |
| | | CTTCCGAGCACCTCGGAGCTCTGATCCAGGGTCCAGGTTATGCCGT |
| | | CCTCTTCTGGCGTATCGCAAGTCAGCACGACCATTTCTCCAGGGGC |
| | | GTCCGGGTACCAATCCAGCTCGACCACGTAGACGTCCTTCTTCAGT |
| | | TCCCAAATGGCGACCAGAGGGGAAGCGAGGAACACAAGGGAGAA |
| | | CCAGGAGATGACGAGTTGCTGATGGCACATCATGGTGGCGACACC |
| | | GGTACGCGTTGGCCCCCATTATATACCCTCTAGAACTAGTtatccactcc |
| | | gtgtaagggagagtgagcctcttacgaatcTTCGGCGTCGACTGCTTCattccgaggcgactg |
| | | ataccTTCGGCGTCGACTGCTTCatacgaaggcagtccgattcTTCGGCGTCGACT |
| | | GCTTCaacctttactgagacgggacTTCGGCGTCGACTGCTTCaaaggcgttgcgaatcc |
| | | tcatgcgattgttacgaaacccgTTAATTAAAGAGCGAGATTCCGTCTCAAAGA |
| | | AAAAAAAAGTAATGAAATGAATAAAATGAGTCCTAGAGCCAGTA |
| | | AATGTCGTAAATGTCTCAGCTAGTCAGGTAGTAAAAGGTCTCAACT |
| | | AGGCAGTGGCAGAGCAGGATTCAAATTCAGGGCTGTTGTGATGCC |
| | | TCCGCAGACTCTGAGCGCCACCTGGTGGTAATTTGTCTGTGCCTCT |
| | | TCTGACGTGGAAGAACAGCAACTAACACACTAACACGGCATTTAC |
| | | TATGGGCCAGCCATTGTCCATCTAGATGGccgataaaataaaagattttatttagtctc |
| | | cagaaaaaggggggaatgaaagaccccacctgtaggtttggcaagctagctgcagtaacgccattttg |
| | | caaggcatggaaaaataccaaaccaagaatagagaagttcagatcaagggcgggtacatgaaaata |
| | | gctaacgttgggccaaacaggatatctgcggtgagcagtttcggccccggcccggggccaagaacag |
| | | atggtcaccgcagtttcggccccggcccgaggccaagaacagatggtccccagatatggcccaaccc |
| | | tcagcagtttcttaagacccatcagatgtttccaggctcccccaaggacctgaaatgaccctgcgcct |
| | | tatttgaattaaccaatcagcctgcttctcgcttctgttcgcgcgcttctgcttcccgagctctataa |
| | | aagagctcacaacccctcactcggcgcgccagtcctccgacagactgagtcgcccgggGGATCCGCC |
| | | ACCATGTCTAGACCTGGCGAGAGGCCCTTCCAGTGCCGGATCTGCATGCGGAACTTCAGCAACATG |
| | | AGCAACCTGACCAGACACACCCGGACACACACAGGCGAGAAGCC |
| | | TTTTCAGTGCAGAATCTGTATGCGCAATTTCTCCGACAGAAGCGTG |
| | | CTGCGGAGACACCTGAGAACCCACACCGGCAGCCAGAAACCATTC |
| | | CAGTGTCGCATCTGTATGAGAAACTTTAGCGACCCCTCCAATCTGG |
| | | CCCGGCACACCAGAACACATACCGGGGAAAAACCCTTTCAGTGTA |
| | | GGATATGCATGAGGAATTTTTCCGACCGGTCCAGCCTGAGGCGGC |
| | | ACCTGAGGACACATACTGGCTCCCAAAAGCCGTTCCAATGTCGGA |
| | | TATGTATGCGCAACTTTAGCCAGAGCGGCACCCTGCACAGACACA |
| | | CAAGAACCCATACTGGCGAGAAACCTTTCCAATGTAGAATCTGCA |
| | | TGCGAAATTTTTCCCAGCGGCCTAATCTGACCAGGCATCTGAGGAC |
| | | CCACCTGAGAGGATCTaCCTGCAGGGATGAGTTTCCCACCATGGTG |
| | | TTTCCTTCTGGGCAGATCAGCCAGGCCTCGGCCTTGGCCCCGGCCC |
| | | CTCCCCAAGTCCTGCCCCAGGCTCCAGCCCCTGCCCCTGCTCCAGC |
| | | CATGGTATCAGCTCTGGCCCAGGCCCCAGCCCCTGTCCCAGTCCTA |
| | | GCCCCAGGCCCTCCTCAGGCTGTGGCCCCACCTGCCCCCAAGCCCA |
| | | CCCAGGCTGGGGAAGGAACGCTGTCAGAGGCCCTGCTGCAGCTGC |
| | | AGTTTGATGATGAAGACCTGGGGGCCTTGCTTGGCAACAGCACAG |
| | | ACCCAGCTGTGTTCACAGACCTGGCATCCGTCGACAACTCCGAGTT |
| | | TCAGCAGCTGCTGAACCAGGGCATACCTGTGGCCCCCCACACAAC |
| | | TGAGCCCATGCTGATGGAGTACCCTGAGGCTATAACTCGCCTAGTG |
| | | ACAGGGGCCCAGAGGCCCCCCGACCCAGCTCCTGCTCCACTGGGG |
| | | GCCCCGGGGCTCCCCAATGGCCTCCTTTCAGGAGATGAAGACTTCT |
| | | CCTCCATTGCGGACATGGACTTCTCAGCCCTGCTGAGTCAGATCAG |
| | | CTCCcaattgtgcgtacgcggatcctctgctggagacatgagagctgccaacctttggccaagcccgc |
| | | tcatgatcaaacgctctaagaagaacagcctggccttgtccctgacggccgaccagatggtcagtgcc |
| | | ttgttggatgctgagccccccatactctattccgagtatgatcctaccagacccttcagtgaagctt |
| | | cgatgatgggcttactgaccaacctggcagacagggagCTGGTTCACATGATCAACTGGGCGAAGAGG |
| | | GTGCCAGGCTTTGTGGATTTGACCCTCCATGATCAGGTCCACCTTCTA |
| | | GAATGTGCCTGGCTAGAGATCCTGATGATTGGTCTCGTCTGGCGCT |
| | | CCATGGAGCACCCAGTGAAGCTACTGTTTGCTCCTAACTTGGTGTT |
| | | GGACAGGGACGAGGGAAAATGTGTAGAGGGCCTGGTGGAGATCTT |
| | | CGACATGCTGCTGGCTACATCATCTCGGTTCCGCATGATGAATCTG |
| | | CAGGGAGAGGAGTTTGTGTGCCTCAAATCTATTATTTTGCTTAATT |
| | | CTGGAGTGTACACATTTCTGCCCAGCACCCTGAAGTCTCTGGAAGA |
| | | GAAGGACCATATCCACCGAGTCCTGGACAAGATCACAGACACTTT |
| | | GATCCACCTGATGGCCAAGGCAGGCCTGACCCTGCAGCAGCAGCA |
| | | CCAGCGGCTGGCCCAGCTCCTCCTCATCCTCTCCCACATCAGGCAC |
| | | ATGAGTAACAAAGGCATGGAGCTGctgtacagcatgaagtgcaagaacgtggtgccc |
| | | ctctatGaCctgctgctggaggcggcggacgcccaccgcctacatgcgcccactagccgtggaggggc |
| | | atccgtggaggagacggaccaaagccacttggccactgcgggctctacttcatcgcattccttgcaaa |
| | | agtattacatcacgggggaggcagagggtttccctgccacaTaAGTCGACAATCAACCTCtggattac |
| | | aaaatttgtgaaagattgactggtattcttaactatgttgctccttttacgctatgtggatacgctgc |
| | | tttaatgcctttgtatcatgctattgcttcccgtatggctttcattttctcctccttgtataaatcct |
| | | ggttgctgtctctttatgaggagttgtggcccgttgtcaggcaacgtggcgtggtgtgcactgtgttt |
| | | gctgacgcaacccccactggttggggcattgccaccacctgtcagctcctttccgggactttcgcttt |
| | | ccccctccctattgccacggcggaactcatcgccgcctgccttgcccgctgctggacaggggctcggc |
| | | tgttgggcactgacaattccgtggtgttgtcggggaaatcatcgtcctttccttggctgctcgcctg |
| | | tgttgccacctggattctgcgcgggacgtccttctgctacgtcccttcggccctcaatccagcggac |
| | | cttccttcccgcggcctgctgccggctctgcggcctcttccgcgtctacgccttcgccctcagacga |
| | | gtcggatctccctttgggccgcctccccgcgatatcagtggtccaggctctagttttgactcaacaa |
| | | tatcaccagctgaagcctatagagtacgagccatagataaaataaaagattttatttagtctccagaa |
| | | aaaggggggaatgaaagaccccacctgtaggtttggcaagctagcaataaaagagcccacaacccctc |
| | | actcggggcgccagtcctccgattgactgagtcgcccggccgcttcgagcagacatgataagatacat |
| | | tgatgagtttggacaaaccacaactagaatgcagtgaaaaaaatgctttatttgtgaaatttgtgat |
| | | gctattgctttatttgtaaccattataagctgcaataaacaagttaacaacaacaattgcattcatt |
| | | ttatgtttcaggttcagggggagatgtgggaggttttttaaagcaagtaaaacctctacaaatgtggt |
| | | aaaatcgataaggatcgggtacccgtgtatccaataaaccctcttgcagttgcatccgacttgtggtc |
| | | tcgctgttccttgggagggtctcctctgagtgattgactacccgtcagcgggggtctttcacacatgc |
| | | agcatgtatcaaaattaatttggttttttttcttaagctgtgccttctagttgccagccatctgttgt |
| | | ttgcccctcccccgtgccttccttgaccctggaaggtgccactcccactgtcctttcctaataaaatg |
| | | aggaaattgcatcgcattgtctgagtaggtgtcattctattctggggggtggggtggggcaggacag |
| | | caagggggaggattgggaagacaatagcaggcatgctggggatgcggtgggctctatggagatcccg |
| | | cggtacctcgcgaatgcatctagatccaatggcctttttggcccagacatgataagatacattgatg |
| | | agtttggacaaaccacaactagaatgcagtgaaaaaaatgctttatttgtgaaatttgtgatgctat |
| | | tgctttatttgtaaccattataagctgcaataaacaagttgcggccgcttagccctcccacacataac |
| | | cagagggcagcaattcacgaatcccaactgccgtcggctgtccatcactgtccttcactatggctttg |
| | | atcccaggatgcagatcgagaagcacctgtcggcaccgtccgcaggggctcaagatgcccctgttctc |
| | | atttccgatcgcgacgatacaagtcaggttgccagctgccgcagcagcagcagtgcccagcaccac |
| | | gagttctgcacaaggtcccccagtaaaatgatatacattgacaccagtgaagatgcggccgtcgct |
| | | agagagagctgcgctggcgacgctgtagtcttcagagatggggatgctgttgattgtagccgttgctc |
| | | tttcaatgagggtggattcttcttgagacaaaggcttggccatgcggccgccgctcggtgttcgagg |
| | | ccacacgcgtcaccttaatatgcgaagtggacctcggaccgcgccgccccgactgcatctgcgtgttc |
| | | gaattcgccaatgacaagacgctgggcggggtttgtgtcatcatagaactaaagacatgcaaatatat |
| | | ttcttccggggggtaccggcctttttggccATTGGatcggatctggccaaaaaggcccttaagtat |
| | | ttacattaaatggccatagtacttaaagttacattggcttccttgaaataaacatggagtattcagaa |
| | | tgtgtcataaatatttctaattttaagatagtatctccattggctttctactttttcttttatttttt |
| | | tttgtcctctgtcttccatttgttgttgttgttgtttgtttgtttgtttgttggttggttggttaat |
| | | ttttttttaaagatcctacactatagttcaagctagactattagctactctgtaacccagggtgacct |
| | | tgaagtcatgggtagcctgctgttttagccttcccacatctaagattacaggtatgagctatcatttt |
| | | tggtatattgattgattgattgattgatgtgtgtgtgtgtgattgtgtttgtgtgtgtgaTtgtgTaT |
| | | atgtgtgtatggTtgtgtgtgaTtgtgtgtatgtatgTTtgtgtgtgaTtgTgtgtgtgtgaTtgtgc |
| | | atgtgtgtgtgtgtgaTtgtgtTtatgtgtatgaTtgtgtgtgtgtgtgtgtgtgtgtgtgtgtgtgt |
| | | gtgtgtgtgtgtgttgtgTaTaTatatttatggtagtgagagGcaacgctccggctcaggtgtcaggt |
| | | tggtttttgagacagagtctttcacttagcttggaattcactggccgtcgttttacaacgtcgtgact |
| | | gggaaaaccctggcgttacccaacttaatcgccttgcagcacatccccctttcgccagctggc |
| | | gtaatagcgaagaggcccgcaccgatcgcccttcccaacagttgcgcagcctgaatggcgaatggcg |
| | | cctgatgcggtattttctccttacgcatctgtgcggtatttcacaccgcatatggtgcactctcagta |
| | | caatctgctctgatgccgcatagttaagccagccccgacacccgccaacacccgctgacgcgccctga |
| | | cgggcttgtctgctcccggcatccgcttacagacaagctgtgaccgtctccgggagctgcatgtgtca |
| | | gaggttttcaccgtcatcaccgaaacgcgcgagacgaaagggcctcgtgatacgcctatttttatagg |
| | | ttaatgtcatgataataatggtttcttagacgtcaggtggcacttttcggggaaatgtgcgcggaacc |
| | | cctatttgtttatttttctaaatacattcaaatatgtatccgctcatgagacaataaccctgataaat |
| | | gcttcaataatattgaaaaaggaagagtatgagtattcaacatttccgtgtcgcccttattccctttt |
| | | ttgcggcattttgccttcctgtttttgctcacccagaaacgctggtgaaagtaaaagatgctga |
| | | agatcagttgggtgcacgagtgggttacatcgaactggatctcaacagcggtaagatccttgagagt |
| | | tttcgccccgaagaacgttttccaatgatgagcacttttaaagttctgctatgtggcgcggtattat |
| | | cccgtattgacgccgggcaagagcaactcggtcgccgcatacactattctcagaatgacttggttga |
| | | gtactcaccagtcacagaaaagcatcttacggatggcatgacagtaagagaattatgcagtgctgcc |
| | | ataaccatgagtgataacactgcggccaacttacttctgacaacgatcggaggaccgaaggagctaac |
| | | cgcttttttgcacaacatgggggatcatgtaactcgccttgatcgttgggaaccggagctgaatgaa |
| | | gccataccaaacgacgagcgtgacaccacgatgcctgtagcaatggcaacaacgttgcgcaaacta |
| | | ttaactggcgaactacttactctagcttcccggcaacaattaatagactggatggaggcggataaag |
| | | ttgcaggaccacttctgcgctcggcccttccggctggctggtttattgctgataaatctggagccgg |
| | | tgagcgtgggtctcgcggtatcattgcagcactggggccagatggtaagccctcccgtatcgtagt |
| | | tatctacacgacggggagtcaggcaactatggatgaacgaaatagacagatcgctgagataggtgcct |
| | | cactgattaagcattggtaactgtcagaccaagtttactcatatatactttagattgatttaaaact |
| | | tcatttttaatttaaaaggatctaggtgaagatcctttttgataatctcatgaccaaaatcccttaac |
| | | gtgagttttcgttccactgagcgtcagaccccgtagaaaagatcaaaggatcttcttgagatccttt |
| | | ttttctgcgcgtaatctgctgcttgcaaacaaaaaaaccaccgctaccagcggtggtttgtttgccg |
| | | gatcaagagctaccaactctttttccgaaggtaactggcttcagcagagcgcagataccaaatactg |
| | | tccttctagtgtagccgtagttaggccaccacttcaagaactctgtagcaccgcctacatacctcgc |
| | | tctgctaatcctgttaccagtggctgctgccagtggcgataagtcgtgtcttaccgggttggactca |
| | | agacgatagttaccggataaggcgcagcggtcgggctgaacggggggttcgtgcacacagcccag |
| | | cttggagcgaacgacctacaccgaactgagatacctacagcgtgagctatgagaaagcgccacgctt |
| | | cccgaagggagaaaggcggacaggtatccggtaagcggcagggtcggaacaggagagcgcacgaggg |
| | | agcttccagggggaaacgcctggtatctttatagtcctgtcgggtttcgccacctctgacttgagcg |
| | | tcgatttttgtgatgctcgtcaggggggcggagcctatggaaaaacgccagcaacgcggccttttta |
| | | cggttcctggccttttgctggccttttgctcacatgttctttcctgcgttatcccctgattctgtgga |
| | | taaccgtattaccgcctttgagtgagctgataccgctcgccgcagccgaacgaccgagcgcagcgag |
| | | tcagtgagcgaggaagcggaagagcgcccaatacgcaaaccgcctctccccgcgcgttggccgattc |
| | | attaatgcagctggcacgacaggtttcccgactggaaagcgggcagtgagcgcaacgcaattaatgt |
| | | gagttagctcactcattaggcaccccaggctttacactttatgcttccggctcgtatgttgtgtgga |
| | | attgtgagcggataacaatttcacacaggaaacagctatgaccatgattacgcc |
| LR1 split N | 74 | AGCGGCGGAGGTGGTAGCGGAGGCGGAGGATCTGGAATTACACA |
| term linker + | | GGGACTCGCCGTGTCTACAATCTCCAGCTTCTTTGGTGGCGGTAGT |
| CD16Tace | | GGCGGCGGTGGCAGTGGCGGTGGATCTCTTCAA |
| (cleavage site) | | |
| LR1 split N | 75 | SGGGGSGGGGSGITQGLAVSTISSFFGGGSGGGGSGGGSLQ |
| term linker + | | |
| CD16Tace | | |
| (cleavage site) | | |
| B7-1 (TM | 76 | CTGCTGCCAAGCTGGGCCATCACACTGATCTCCGTGAACGGCATCT |
| domain) | | TCGTGATCTGTTGCCTGACCTACTGCTTCGCCCCTCGGTGCAGAGA |
| | | GCGGAGAAGAAACGAACGGCTGCGGAGAGAATCTGTGCGGCCTGT |
| | | G |
| B7-1 (TM | 77 | LLPSWAITLISVNGIFVICCLTYCFAPRCRERRRNERLRRESVRPV |
| domain) | | |
| SV40 promoter | 78 | GTGTGTCAGTTAGGGTGTGGAAAGTCCCCAGGCTCCCCAGCAGGC |
| | | AGAAGTATGCAAAGCATGCATCTCAATTAGTCAGCAACCAGGTGT |
| | | GGAAAGTCCCCAGGCTCCCCAGCAGGCAGAAGTATGCAAAGCATG |
| | | CATCTCAATTAGTCAGCAACCATAGTCCCGCCCCTAACTCCGCCCA |
| | | TCCCGCCCCTAACTCCGCCCAGTTCCGCCCATTCTCCGCCCCATGG |
| | | CTGACTAATTTTTTTTATTTATGCAGAGGCCGAGGCCGCCTCTGCC |
| | | TCTGAGCTATTCCAGAAGTAGTGAGGAGGCTTTTTTGGAGGCCTAG |
| | | GCTTTTGCAAA |
| SB07127 vector | 79 | aagcttgaattcgagcttgcatgcctgcaggtcgttacataacttacggtaaatggcccgcctggctg |
| | | accgcccaacgacccccgcccattgacgtcaataatgacgtatgttcccatagtaacgccaataggga |
| | | ctttccattgacgtcaatgggtggagtatttacggtaaactgcccacttggcagtacatcaagtgtat |
| | | catatgccaagtacgccccctattgacgtcaatgacggtaaatggcccgcctggcattatgcccagta |
| | | catgaccttatgggactttcctacttggcagtacatctacgtattagtcatcgctattaccatggtga |
| | | tgcggttttggcagtacatcaatgggcgtggatagcggtttgactcacggggatttccaagtctccac |
| | | cccattgacgtcaatgggagtttgttttggcaccaaaatcaacgggactttccaaaatgtcgtaaca |
| | | actccgccccattgacgcaaatgggcggtaggcgtgtacggtgggaggtctatataagcagagctca |
| | | ataaaagagcccacaacccctcactcggcgcgccagtcctccgattgactgagtcgcccgggtacc |
| | | cgtgtatccaataaaccctcttgcagttgcatccgacttgtggtctcgctgttccttgggagggtc |
| | | tcctctgagtgattgactacccgtcagcgggggtctttcatttgggggctcgtccgagatcgggagac |
| | | ccctgcccagggaccaccgacccaccaccgggaggtaagctggccagcaacttatctgtgtctgtccg |
| | | attgtctagtgtctatgactgattttatgcgcctgcgtcggtactagttagctaactagctctgtat |
| | | ctggcggacccgtggtggaactgacgagttcggaacacccggccgcaaccctgggagacgtcccaggg |
| | | acttcgggggccgtttttgtggcccgacctgagtcctaaaatcccgatcgtttaggactctttggtgc |
| | | accccccttagaggagggatatgtggttctggtaggagacgagaacctaaaacagttcccgcctccgt |
| | | ctgaatttttgctttcggtttgggaccgaagccgcgccgcgcgtcttgtctgctgcagcatcgttct |
| | | gtgttgtctctgtctgactgtgtttctgtatttgtctgaaaatatgggccccccctcgagtccccagc |
| | | atgcctgctattctcttcccaatcctcccccttgctgtcctgccccaccccaccccccagaatagaa |
| | | tgacacctactcagacaatgcgatgcaatttcctcattttattaggaaaggacagtgggagtggcac |
| | | cttccagggtcaaggaaggcacgggggaggggcaaacaacagatggctggcaactagaaggcacagtt |
| | | acttaCACAGGCCGCACAGATTCTCTCCGCAGCCGTTCGTTTCTTCTCCG |
| | | CTCTCTGCACCGAGGGGCGAAGCAGTAGGTCAGGCAACAGATCAC |
| | | GAAGATGCCGTTCACGGAGATCAGTGTGATGGCCCAGCTTGGCAG |
| | | CAGTTGAAGAGATCCACCGCCACTGCCACCGCCGCCACTACCGCC |
| | | ACCAAAGAAGCTGGAGATTGTAGACACGGCGAGTCCCTGTGTAAT |
| | | TCCAGATCCTCCGCCTCCGCTACCACCTCCGCCGCTAGAGGCGTTC |
| | | AGGTAGCTCATCACTCTGTCGATGGTCACGGCTCTGATCCGGAAGG |
| | | CGTGCAGCAGGATGCACAGCTTGATCTTGGTCTTGTAGAAGTCGG |
| | | GTTCTTCCAGGCTAGACTTCTGGGGCACTGTCTCGCTGTTGAAGTT |
| | | CAGGGCCTGCATCAGCTCGTCGATCACGGCCAGCATATTCTGGTCC |
| | | AGGAAGATCTGCCGCTTGGGGTCCATCAGCAGCTTGGCGTTCATG |
| | | GTCTTGAATTCCACCTGGTACATCTTCAGGTCCTCGTAGATGCTGC |
| | | TCAGGCACAGGGCCATCATGAAGGAGGTCTTTCTGCTGGCCAGGC |
| | | AAGAGCCGTTGGTGATGAAGCTGGTTTCCCGGCTGTTCAGGCAGCT |
| | | CTCGTTCTTGGTCAGTTCCAGAGGCAGGCAGGCTTCCACGGTGCTG |
| | | GTCTTATCCTTGGTGATGTCCTCGTGGTCGATTTCCTCGCTGGTGCA |
| | | GGGGTAGAATTCCAGGGTCTGTCTGGCCTTCTGCAGCATGTTGGAC |
| | | ACGGCTCTCAGCAGGTTCTGGCTGTGGTGCAGACAAGGGAACATG |
| | | CCAGGATCAGGAGTGGCCACAGGCAGGTTTCTAGATCCGCCGCCA |
| | | GATCCACCACCTGATCCGCCACCGCTTCCTCCGCCAGAACATGGCA |
| | | CGCTGGCCCATTCGCTCCAAGAGCTGCTGTAGTACCGGTCCTGGGC |
| | | TCTGACGCTGATGCTGGCGTTCTTTCTGCAGATCACGGTGGCGCTG |
| | | GTCTTGTCGGTGAACACCCGGTCCTTTTTCTCGCGCTTGGACTTGCC |
| | | CTGCACTTGCACGCAAAAGGTCAGGCTGAAGTAGCTGTGGGGTGT |
| | | AGACCAGGTGTCGGGGTACTCCCAGGACACTTCCACCTGTCTGCTG |
| | | TTCTTCAGAGGCTTCAGCTGCAGGTTCTTTGGAGGATCGGGCTTGA |
| | | TGATGTCCCGGATGAAAAAGCTGGAGGTGTAGTTCTCGTACTTCAG |
| | | CTTGTGCACGGCGTCCACCATCACTTCGATAGGCAGAGACTCTTCG |
| | | GCGGCTGGACAGGCGCTGTCCTCTTGGCATTCCACGCTGTACTCGT |
| | | ATTCTTTGTTGTCGCCCCGCACTCTTTCGGCAGACAGTGTAGCGGC |
| | | GCCACATGTAACGCCCTGAGGATCACTGCTGCCTCTGCTGGACTTC |
| | | ACGCTGAAGGTCAGGTCGGTGCTGATGGTGGTCAGCCACCAACAT |
| | | GTGAACCGGCCGCTGTAGTTCTTGGCCTCGCATCTCAGGAAGGTCT |
| | | TGTTCTTGGGCTCTTTCTGGTCCTTCAGGATGTCGGTGCTCCAAATG |
| | | CCATCCTCTTTCTTGTGGAGCAGCAGCAGGCTGTGGCTCAGCACTT |
| | | CTCCGCCTTTGTGACAGGTGTACTGGCCGGCGTCGCCAAACTCTTT |
| | | CACTTGGATGGTCAGGGTCTTGCCGCTGCCGAGCACCTCGCTAGAC |
| | | TGATCCAGTGTCCAGGTGATGCCGTCCTCTTCAGGGGTATCGCAGG |
| | | TCAGCACCACCATCTCGCCAGGAGCATCGGGATACCAGTCCAGTT |
| | | CCACCACGTACACGTCTTTCTTCAGCTCCCAGATGGCCACCAGAGG |
| | | AGAGGCCAGGAACACCAGGCTGAACCAGCTGATGACCAGCTGCTG |
| | | GTGACACATCATGGTGGCGACACCGGTACGCGTTGGCCCCCATTAT |
| | | ATACCCTCTAGAACTAGTtatccactccgtgtaagggagagtgagcctcttacgaatcTTC |
| | | GGCGTCGACTGCTTCattccgaggcgactgataccTTCGGCGTCGACTGCTTCat |
| | | acgaaggcagtccgattcTTCGGCGTCGACTGCTTCaacctttactgagacgggacTTCG |
| | | GCGTCGACTGCTTCaaaggcgttgcgaatcctcatgcgattgttacgaaacccgTTAATTA |
| | | AAGAGCGAGATTCCGTCTCAAAGAAAAAAAAAGTAATGAAATGA |
| | | ATAAAATGAGTCCTAGAGCCAGTAAATGTCGTAAATGTCTCAGCT |
| | | AGTCAGGTAGTAAAAGGTCTCAACTAGGCAGTGGCAGAGCAGGAT |
| | | TCAAATTCAGGGCTGTTGTGATGCCTCCGCAGACTCTGAGCGCCAC |
| | | CTGGTGGTAATTTGTCTGTGCCTCTTCTGACGTGGAAGAACAGCAA |
| | | CTAACACACTAACACGGCATTTACTATGGGCCAGCCATTGTCCATC |
| | | TAGATGGccgataaaataaaagattttatttagtctccagaaaaaggggggaatgaaagaccccacct |
| | | gtaggtttggcaagctagctgcaGTGTGTCAGTTAGGGTGTGGAAAGTCCCCAGG |
| | | CTCCCCAGCAGGCAGAAGTATGCAAAGCATGCATCTCAATTAGTC |
| | | AGCAACCAGGTGTGGAAAGTCCCCAGGCTCCCCAGCAGGCAGAAG |
| | | TATGCAAAGCATGCATCTCAATTAGTCAGCAACCATAGTCCCGCCC |
| | | CTAACTCCGCCCATCCCGCCCCTAACTCCGCCCAGTTCCGCCCATT |
| | | CTCCGCCCCATGGCTGACTAATTTTTTTTATTTATGCAGAGGCCGA |
| | | GGCCGCCTCTGCCTCTGAGCTATTCCAGAAGTAGTGAGGAGGCTTT |
| | | TTTGGAGGCCTAGGCTTTTGCAAAGGATCCGCCACCATGTCTAGAC |
| | | CTGGCGAGAGGCCCTTCCAGTGCCGGATCTGCATGCGGAACTTCA |
| | | GCAACATGAGCAACCTGACCAGACACACCCGGACACACACAGGC |
| | | GAGAAGCCTTTTCAGTGCAGAATCTGTATGCGCAATTTCTCCGACA |
| | | GAAGCGTGCTGCGGAGACACCTGAGAACCCACACCGGCAGCCAG |
| | | AAACCATTCCAGTGTCGCATCTGTATGAGAAACTTTAGCGACCCCT |
| | | CCAATCTGGCCCGGCACACCAGAACACATACCGGGGAAAAACCCT |
| | | TTCAGTGTAGGATATGCATGAGGAATTTTTCCGACCGGTCCAGCCT |
| | | GAGGCGGCACCTGAGGACACATACTGGCTCCCAAAAGCCGTTCCA |
| | | ATGTCGGATATGTATGCGCAACTTTAGCCAGAGCGGCACCCTGCA |
| | | CAGACACACAAGAACCCATACTGGCGAGAAACCTTTCCAATGTAG |
| | | AATCTGCATGCGAAATTTTTCCCAGCGGCCTAATCTGACCAGGCAT |
| | | CTGAGGACCCACCTGAGAGGATCTaCCTGCAGGGATGAGTTTCCCA |
| | | CCATGGTGTTTCCTTCTGGGCAGATCAGCCAGGCCTCGGCCTTGGC |
| | | CCCGGCCCCTCCCCAAGTCCTGCCCCAGGCTCCAGCCCCTGCCCCT |
| | | GCTCCAGCCATGGTATCAGCTCTGGCCCAGGCCCCAGCCCCTGTCC |
| | | CAGTCCTAGCCCCAGGCCCTCCTCAGGCTGTGGCCCCACCTGCCCC |
| | | CAAGCCCACCCAGGCTGGGGAAGGAACGCTGTCAGAGGCCCTGCT |
| | | GCAGCTGCAGTTTGATGATGAAGACCTGGGGGCCTTGCTTGGCAA |
| | | CAGCACAGACCCAGCTGTGTTCACAGACCTGGCATCCGTCGACAA |
| | | CTCCGAGTTTCAGCAGCTGCTGAACCAGGGCATACCTGTGGCCCCC |
| | | CACACAACTGAGCCCATGCTGATGGAGTACCCTGAGGCTATAACT |
| | | CGCCTAGTGACAGGGGCCCAGAGGCCCCCCGACCCAGCTCCTGCT |
| | | CCACTGGGGGCCCCGGGGCTCCCCAATGGCCTCCTTTCAGGAGAT |
| | | GAAGACTTCTCCTCCATTGCGGACATGGACTTCTCAGCCCTGCTGA |
| | | GTCAGATCAGCTCCcaattgtgcgtacgcggatcctctgctggagacatgagagctgccaaccttt |
| | | ggccaagcccgctcatgatcaaacgctctaagaagaacagcctggccttgtccctgacggccgaccag |
| | | atggtcagtgccttgttggatgctgagccccccatactctattccgagtatgatcctaccagaccctt |
| | | cagtgaagcttcgatgatgggcttactgaccaacctggcagacagggagCTGGTTCACATGATCAACT |
| | | GGGCGAAGAGGGTGCCAGGCTTTGTGGATTTGACCCTCCATGATCAGGT |
| | | CCACCTTCTAGAATGTGCCTGGATGGAGATCCTGATGATTGGTGTG |
| | | GTCTGGCGCTCCATGGAGCACCCAGTGAAGCTACTGTTTGCTCCTA |
| | | ACTTGCTCTTGGACAGGGACCAGGGAAAATGTGTAGAGGGCCTGG |
| | | TGGAGATCTTCGACATGCTGCTGGCTACATCATCTCGGTTCCGCAT |
| | | GATGAATCTGCAGGGAGAGGAGTTTGTGTGCCTCAAATCTATTATT |
| | | TTGCTTAATTCTGGAGTGTACACATTTCTGCCCAGCACCCTGAAGT |
| | | CTCTGGAAGAGAAGGACCATATCCACCGAGTCCTGGACAAGATCA |
| | | CAGACACTTTGATCCACCTGATGGCCAAGGCAGGCCTGACCCTGC |
| | | AGCAGCAGCACCAGCGGCTGGCCCAGCTCCTCCTCATCCTCTCCCA |
| | | CATCAGGCACATGAGTAACAAAGGCATGGAGCTGctgtacagcatgaagtgc |
| | | aagaacgtggtgcccctctatGaCctgctgctggaggcggcggacgcccaccgcctacatgcgccca |
| | | ctagccgtggaggggcatccgtggaggagacggaccaaagccacttggccactgcgggctctacttca |
| | | tcgcattccttgcaaaagtattacatcacgggggaggcagagggtttccctgccacaTaAGTCGACA |
| | | ATCAACCTCtggattacaaaatttgtgaaagattgactggtattcttaactatgttgctccttttacg |
| | | ctatgtggatacgctgctttaatgcctttgtatcatgctattgcttcccgtatggctttcattttctc |
| | | ctccttgtataaatcctggttgctgtctctttatgaggagttgtggcccgttgtcaggcaacgtggc |
| | | gtggtgtgcactgtgtttgctgacgcaacccccactggttggggcattgccaccacctgtcagctcc |
| | | tttccgggactttcgctttccccctccctattgccacggcggaactcatcgccgcctgccttgcccgc |
| | | tgctggacaggggctcggctgttgggcactgacaattccgtggtgttgtcggggaaatcatcgtcctt |
| | | tccttggctgctcgcctgtgttgccacctggattctgcgcgggacgtccttctgctacgtcccttcg |
| | | gccctcaatccagggaccttccttcccgcggcctgctgccggctctgcggcctcttccgcgtctacg |
| | | ccttcgccctcagacgagtcggatctccctttgggccgcctccccgcgatatcagtggtccaggctct |
| | | agttttgactcaacaatatcaccagctgaagcctatagagtacgagccatagataaaaaaaagattt |
| | | tatttagtctccagaaaaaggggggaatgaaagaccccacctgtaggtttggcaagctagcaataaa |
| | | agagcccacaacccctcactcggggcgccagtcctccgattgactgagtcgcccggccgcttcgagca |
| | | gacatgataagatacattgatgagtttggacaaaccacaactagaatgcagtgaaaaaaatgcttt |
| | | atttgtgaaatttgtgatgctattgctttatttgtaaccattataagctgcaataaacaagttaacaa |
| | | caacaattgcattcattttatgtttcaggttcagggggagatgtgggaggttttttaaagcaagtaaa |
| | | acctctacaaatgtggtaaaatcgataaggatcgggtacccgtgtatccaataaaccctcttgcagtt |
| | | gcatccgacttgtggtctcgctgttccttgggagggtctcctctgagtgattgactacccgtca |
| | | gcgggggtctttcacacatgcagcatgtatcaaaattaatttggttttttttcttaagctgtgcctt |
| | | ctagttgccagccatctgttgtttgcccctcccccgtgccttccttgaccctggaaggtgccactcc |
| | | cactgtcctttcctaataaaatgaggaaattgcatcgcattgtctgagtaggtgtcattctattct |
| | | ggggggtggggggggcaggacagcaagggggaggattgggaagacaatagcaggcatgctggggat |
| | | gcggtgggctctatggagatcccgcggtacctcgcgaatgcatctagatccaatggcctttttggccc |
| | | agacatgataagatacattgatgagtttggacaaaccacaactagaatgcagtgaaaaaaatgcttt |
| | | atttgtgaaatttgtgatgctattgctttatttgtaaccattataagctgcaataaacaagttgcgg |
| | | ccgcttagccctcccacacataaccagagggcagcaattcacgaatcccaactgccgtcggctgtcc |
| | | atcactgtccttcactatggctttgatcccaggatgcagatcgagaagcacctgtcggcaccgtcc |
| | | gcaggggctcaagatgcccctgttctcatttccgatcgcgacgatacaagtcaggttgccagctgc |
| | | cgcagcagcagcagtgcccagcaccacgagttctgcacaaggtcccccagtaaaatgatatacatt |
| | | gacaccagtgaagatgcggccgtcgctagagagagctgcgctggcgacgctgtagtcttcagagatgg |
| | | ggatgctgttgattgtagccgttgctctttcaatgagggtggattcttcttgagacaaaggcttggc |
| | | catgcggccgccgctcggtgttcgaggccacacgcgtcaccttaatatgcgaagtggacctcggacc |
| | | gcgccgccccgactgcatctgcgtgttcgaattcgccaatgacaagacgctgggcggggtttgtgtca |
| | | tcatagaactaaagacatgcaaatatatttcttccggggggtaccggcctttttggccATTGGatcgg |
| | | atctggccaaaaaggcccttaagtatttacattaaatggccatagtacttaaagttacattggcttc |
| | | cttgaaataaacatggagtattcagaatgtgtcataaatatttctaattttaagatagtatctccatt |
| | | ggctttctactttttcttttatttttttttgtcctctgtcttccatttgttgttgttgttgtttgtt |
| | | tgtttgtttgttggttggttggttaatttttttttaaagatcctacactatagttcaagctagacta |
| | | ttagctactctgtaacccagggtgaccttgaagtcatgggtagcctgctgttttagccttcccacatc |
| | | taagattacaggtatgagctatcatttttggtatattgattgattgattgattgatgtgtgtgtgtg |
| | | tgattgtgtttgtgtgtgtgaTtgtgTaTatgtgtgtatggTtgtgtgtgaTtgtgtgtatgtatgT |
| | | TtgtgtgtgaTtgTgtgtgtgtgaTtgtgcatgtgtgtgtgtgtgaTtgtgtTtatgtgtatgaTtg |
| | | tgtgtgtgtgtgtgtgtgtgtgtgtgtgtgtgtgtgtgtgtgtgttgtgTaTaTatatttatggta |
| | | gtgagagGcaacgctccggctcaggtgtcaggttggtttttgagacagagtctttcacttagcttgga |
| | | attcactggccgtcgttttacaacgtcgtgactgggaaaaccctggcgttacccaacttaatcgcctt |
| | | gcagcacatccccctttcgccagctggcgtaatagcgaagaggcccgcaccgatcgcccttcccaaca |
| | | gttgcgcagcctgaatggcgaatggcgcctgatgcggtattttctccttacgcatctgtgcggtattt |
| | | cacaccgcatatggtgcactctcagtacaatctgctctgatgccgcatagttaagccagccccgac |
| | | acccgccaacacccgctgacgcgccctgacgggcttgtctgctcccggcatccgcttacagacaagct |
| | | gtgaccgtctccgggagctgcatgtgtcagaggttttcaccgtcatcaccgaaacgcgcgagacgaaa |
| | | gggcctcgtgatacgcctatttttataggttaatgtcatgataataatggtttcttagacgtcaggt |
| | | ggcacttttcggggaaatgtgcgcggaacccctatttgtttatttttctaaatacattcaaatatgt |
| | | atccgctcatgagacaataaccctgataaatgcttcaataatattgaaaaaggaagagtatgagtat |
| | | tcaacatttccgtgtcgcccttattcccttttttgcggcattttgccttcctgtttttgctcaccca |
| | | gaaacgctggtgaaagtaaaagatgctgaagatcagttgggtgcacgagtgggttacatcgaactgg |
| | | atctcaacagcggtaagatccttgagagttttcgccccgaagaacgttttccaatgatgagcactt |
| | | ttaaagttctgctatgtggcgcggtattatcccgtattgacgccgggcaagagcaactcggtcgccg |
| | | catacactattctcagaatgacttggttgagtactcaccagtcacagaaaagcatcttacggatggca |
| | | tgacagtaagagaattatgcagtgctgccataaccatgagtgataacactgcggccaacttacttct |
| | | gacaacgatcggaggaccgaaggagctaaccgcttttttgcacaacatgggggatcatgtaactcgc |
| | | cttgatcgttgggaaccggagctgaatgaagccataccaaacgacgagcgtgacaccacgatgcctg |
| | | tagcaatggcaacaacgttgcgcaaactattaactggcgaactacttactctagcttcccggcaacaa |
| | | ttaatagactggatggaggcggataaagttgcaggaccacttctgcgctcggcccttccggctggctg |
| | | gtttattgctgataaatctggagccggtgagcgtgggtctcgcggtatcattgcagcactggggccag |
| | | atggtaagccctcccgtatcgtagttatctacacgacggggagtcaggcaactatggatgaacgaaat |
| | | agacagatcgctgagataggtgcctcactgattaagcattggtaactgtcagaccaagtttactcat |
| | | atatactttagattgatttaaaacttcatttttaatttaaaaggatctaggtgaagatcctttttg |
| | | ataatctcatgaccaaaatcccttaacgtgagttttcgttccactgagcgtcagaccccgtagaaaag |
| | | atcaaaggatcttcttgagatcctttttttctgcgcgtaatctgctgcttgcaaacaaaaaaaccacc |
| | | gctaccagcggtggtttgtttgccggatcaagagctaccaactctttttccgaaggtaactggcttc |
| | | agcagagcgcagataccaaatactgtccttctagtgtagccgtagttaggccaccacttcaagaact |
| | | ctgtagcaccgcctacatacctcgctctgctaatcctgttaccagtggctgctgccagtggcgataag |
| | | tcgtgtcttaccgggttggactcaagacgatagttaccggataaggcgcagcggtcgggctgaacggg |
| | | gggttcgtgcacacagcccagcttggagcgaacgacctacaccgaactgagatacctacagcgtgag |
| | | ctatgagaaagcgccacgcttcccgaagggagaaaggcggacaggtatccggtaagcggcagggtcgg |
| | | aacaggagagcgcacgagggagcttccagggggaaacgcctggtatctttatagtcctgtcgggtttc |
| | | gccacctctgacttgagcgtcgatttttgtgatgctcgtcaggggggcggagcctatggaaaaacgc |
| | | cagcaacgcggcctttttacggttcctggccttttgctggccttttgctcacatgttctttcctgcg |
| | | ttatcccctgattctgtggataaccgtattaccgcctttgagtgagctgataccgctcgccgcagc |
| | | cgaacgaccgagcgcagcgagtcagtgagcgaggaagcggaagagcgcccaatacgcaaaccgcctc |
| | | tccccgcgcgttggccgattcattaatgcagctggcacgacaggtttcccgactggaaagcgggcagt |
| | | gagcgcaacgcaattaatgtgagttagctcactcattaggcaccccaggctttacactttatgcttc |
| | | cggctcgtatgttgtgtggaattgtgagcggataacaatttcacacaggaaacagctatgaccatga |
| | | ttacgcc |
| SB07133 vector | 80 | aagcttgaattcgagcttgcatgcctgcaggtcgttacataacttacggtaaatggcccgcctggct |
| | | gaccgcccaacgacccccgcccattgacgtcaataatgacgtatgttcccatagtaacgccaatagg |
| | | gactttccattgacgtcaatgggtggagtatttacggtaaactgcccacttggcagtacatcaagtg |
| | | tatcatatgccaagtacgccccctattgacgtcaatgacggtaaatggcccgcctggcattatgccca |
| | | gtacatgaccttatgggactttcctacttggcagtacatctacgtattagtcatcgctattaccatgg |
| | | tgatgcggttttggcagtacatcaatgggcgtggatagcggtttgactcacggggatttccaagtct |
| | | ccaccccattgacgtcaatgggagtttgttttggcaccaaaatcaacgggactttccaaaatgtcgta |
| | | acaactccgccccattgacgcaaatgggcggtaggcgtgtacggtgggaggtctatataagcagagct |
| | | caataaaagagcccacaacccctcactcggcgcgccagtcctccgattgactgagtcgcccgggtac |
| | | ccgtgtatccaataaaccctcttgcagttgcatccgacttgtggtctcgctgttccttgggagggtc |
| | | tcctctgagtgattgactacccgtcagcgggggtctttcatttgggggctcgtccgagatcgggagac |
| | | ccctgcccagggaccaccgacccaccaccgggaggtaagctggccagcaacttatctgtgtctgtcc |
| | | gattgtctagtgtctatgactgattttatgcgcctgcgtcggtactagttagctaactagctctgta |
| | | tctggcggacccgtggtggaactgacgagttcggaacacccggccgcaaccctgggagacgtcccag |
| | | ggacttcgggggccgtttttgtggcccgacctgagtcctaaaatcccgatcgtttaggactctttgg |
| | | tgcaccccccttagaggagggatatgtggttctggtaggagacgagaacctaaaacagttcccgcct |
| | | ccgtctgaatttttgctttcggtttgggaccgaagccgcgccgcgcgtcttgtctgctgcagcatcg |
| | | ttctgtgttgtctctgtctgactgtgtttctgtatttgtctgaaaatatgggccccccctcgagtccc |
| | | cagcatgcctgctattctcttcccaatcctcccccttgctgtcctgccccaccccaccccccagaata |
| | | gaatgacacctactcagacaatgcgatgcaatttcctcattttattaggaaaggacagtgggagtggc |
| | | accttccagggtcaaggaaggcacgggggaggggcaaacaacagatggctggcaactagaaggcaca |
| | | gttacttaCACAGGCCGCACAGATTCTCTCCGCAGCCGTTCGTTTCTTCTCCG |
| | | CTCTCTGCACCGAGGGGCGAAGCAGTAGGTCAGGCAACAGATCAC |
| | | GAAGATGCCGTTCACGGAGATCAGTGTGATGGCCCAGCTTGGCAG |
| | | CAGTTGAAGAGATCCACCGCCACTGCCACCGCCGCCACTACCGCC |
| | | ACCAAAGAAGCTGGAGATTGTAGACACGGCGAGTCCCTGTGTAAT |
| | | TCCAGATCCTCCGCCTCCGCTACCACCTCCGCCGCTAGAGGCGTTC |
| | | AGGTAGCTCATCACTCTGTCGATGGTCACGGCTCTGATCCGGAAGG |
| | | CGTGCAGCAGGATGCACAGCTTGATCTTGGTCTTGTAGAAGTCGG |
| | | GTTCTTCCAGGCTAGACTTCTGGGGCACTGTCTCGCTGTTGAAGTT |
| | | CAGGGCCTGCATCAGCTCGTCGATCACGGCCAGCATATTCTGGTCC |
| | | AGGAAGATCTGCCGCTTGGGGTCCATCAGCAGCTTGGCGTTCATG |
| | | GTCTTGAATTCCACCTGGTACATCTTCAGGTCCTCGTAGATGCTGC |
| | | TCAGGCACAGGGCCATCATGAAGGAGGTCTTTCTGCTGGCCAGGC |
| | | AAGAGCCGTTGGTGATGAAGCTGGTTTCCCGGCTGTTCAGGCAGCT |
| | | CTCGTTCTTGGTCAGTTCCAGAGGCAGGCAGGCTTCCACGGTGCTG |
| | | GTCTTATCCTTGGTGATGTCCTCGTGGTCGATTTCCTCGCTGGTGCA |
| | | GGGGTAGAATTCCAGGGTCTGTCTGGCCTTCTGCAGCATGTTGGAC |
| | | ACGGCTCTCAGCAGGTTCTGGCTGTGGTGCAGACAAGGGAACATG |
| | | CCAGGATCAGGAGTGGCCACAGGCAGGTTTCTAGATCCGCCGCCA |
| | | GATCCACCACCTGATCCGCCACCGCTTCCTCCGCCAGAACATGGCA |
| | | CGCTGGCCCATTCGCTCCAAGAGCTGCTGTAGTACCGGTCCTGGGC |
| | | TCTGACGCTGATGCTGGCGTTCTTTCTGCAGATCACGGTGGCGCTG |
| | | GTCTTGTCGGTGAACACCCGGTCCTTTTTCTCGCGCTTGGACTTGCC |
| | | CTGCACTTGCACGCAAAAGGTCAGGCTGAAGTAGCTGTGGGGTGT |
| | | AGACCAGGTGTCGGGGTACTCCCAGGACACTTCCACCTGTCTGCTG |
| | | TTCTTCAGAGGCTTCAGCTGCAGGTTCTTTGGAGGATCGGGCTTGA |
| | | TGATGTCCCGGATGAAAAAGCTGGAGGTGTAGTTCTCGTACTTCAG |
| | | CTTGTGCACGGCGTCCACCATCACTTCGATAGGCAGAGACTCTTCG |
| | | GCGGCTGGACAGGCGCTGTCCTCTTGGCATTCCACGCTGTACTCGT |
| | | ATTCTTTGTTGTCGCCCCGCACTCTTTCGGCAGACAGTGTAGCGGC |
| | | GCCACATGTAACGCCCTGAGGATCACTGCTGCCTCTGCTGGACTTC |
| | | ACGCTGAAGGTCAGGTCGGTGCTGATGGTGGTCAGCCACCAACAT |
| | | GTGAACCGGCCGCTGTAGTTCTTGGCCTCGCATCTCAGGAAGGTCT |
| | | TGTTCTTGGGCTCTTTCTGGTCCTTCAGGATGTCGGTGCTCCAAATG |
| | | CCATCCTCTTTCTTGTGGAGCAGCAGCAGGCTGTGGCTCAGCACTT |
| | | CTCCGCCTTTGTGACAGGTGTACTGGCCGGCGTCGCCAAACTCTTT |
| | | CACTTGGATGGTCAGGGTCTTGCCGCTGCCGAGCACCTCGCTAGAC |
| | | TGATCCAGTGTCCAGGTGATGCCGTCCTCTTCAGGGGTATCGCAGG |
| | | TCAGCACCACCATCTCGCCAGGAGCATCGGGATACCAGTCCAGTT |
| | | CCACCACGTACACGTCTTTCTTCAGCTCCCAGATGGCCACCAGAGG |
| | | AGAGGCCAGGAACACCAGGCTGAACCAGCTGATGACCAGCTGCTG |
| | | GTGACACATCATGGTGGCGACACCGGTACGCGTTGGCCCCCATTAT |
| | | ATACCCTCTAGAACTAGTtatccactccgtgtaagggagagtgagcctcttacgaatcTTC |
| | | GGCGTCGACTGCTTCattccgaggcgactgataccTTCGGCGTCGACTGCTTCat |
| | | acgaaggcagtccgattcTTCGGCGTCGACTGCTTCaacctttactgagacgggacTTCG |
| | | GCGTCGACTGCTTCaaaggcgttgcgaatcctcatgcgattgttacgaaacccgTTAATTA |
| | | AAGAGCGAGATTCCGTCTCAAAGAAAAAAAAAGTAATGAAATGA |
| | | ATAAAATGAGTCCTAGAGCCAGTAAATGTCGTAAATGTCTCAGCT |
| | | AGTCAGGTAGTAAAAGGTCTCAACTAGGCAGTGGCAGAGCAGGAT |
| | | TCAAATTCAGGGCTGTTGTGATGCCTCCGCAGACTCTGAGCGCCAC |
| | | CTGGTGGTAATTTGTCTGTGCCTCTTCTGACGTGGAAGAACAGCAA |
| | | CTAACACACTAACACGGCATTTACTATGGGCCAGCCATTGTCCATC |
| | | TAGATGGccgataaaataaaagattttatttagtctccagaaaaaggggggaatgaaagaccccacct |
| | | gtaggtttggcaagctagctgcaGTGTGTCAGTTAGGGTGTGGAAAGTCCCCAGG |
| | | CTCCCCAGCAGGCAGAAGTATGCAAAGCATGCATCTCAATTAGTC |
| | | AGCAACCAGGTGTGGAAAGTCCCCAGGCTCCCCAGCAGGCAGAAG |
| | | TATGCAAAGCATGCATCTCAATTAGTCAGCAACCATAGTCCCGCCC |
| | | CTAACTCCGCCCATCCCGCCCCTAACTCCGCCCAGTTCCGCCCATT |
| | | CTCCGCCCCATGGCTGACTAATTTTTTTTATTTATGCAGAGGCCGA |
| | | GGCCGCCTCTGCCTCTGAGCTATTCCAGAAGTAGTGAGGAGGCTTT |
| | | TTTGGAGGCCTAGGCTTTTGCAAAGGATCCGCCACCATGTCTAGAC |
| | | CTGGCGAGAGGCCCTTCCAGTGCCGGATCTGCATGCGGAACTTCA |
| | | GCAACATGAGCAACCTGACCAGACACACCCGGACACACACAGGC |
| | | GAGAAGCCTTTTCAGTGCAGAATCTGTATGCGCAATTTCTCCGACA |
| | | GAAGCGTGCTGCGGAGACACCTGAGAACCCACACCGGCAGCCAG |
| | | AAACCATTCCAGTGTCGCATCTGTATGAGAAACTTTAGCGACCCCT |
| | | CCAATCTGGCCCGGCACACCAGAACACATACCGGGGAAAAACCCT |
| | | TTCAGTGTAGGATATGCATGAGGAATTTTTCCGACCGGTCCAGCCT |
| | | GAGGCGGCACCTGAGGACACATACTGGCTCCCAAAAGCCGTTCCA |
| | | ATGTCGGATATGTATGCGCAACTTTAGCCAGAGCGGCACCCTGCA |
| | | CAGACACACAAGAACCCATACTGGCGAGAAACCTTTCCAATGTAG |
| | | AATCTGCATGCGAAATTTTTCCCAGCGGCCTAATCTGACCAGGCAT |
| | | CTGAGGACCCACCTGAGAGGATCTaCCTGCAGGGATGAGTTTCCCA |
| | | CCATGGTGTTTCCTTCTGGGCAGATCAGCCAGGCCTCGGCCTTGGC |
| | | CCCGGCCCCTCCCCAAGTCCTGCCCCAGGCTCCAGCCCCTGCCCCT |
| | | GCTCCAGCCATGGTATCAGCTCTGGCCCAGGCCCCAGCCCCTGTCC |
| | | CAGTCCTAGCCCCAGGCCCTCCTCAGGCTGTGGCCCCACCTGCCCC |
| | | CAAGCCCACCCAGGCTGGGGAAGGAACGCTGTCAGAGGCCCTGCT |
| | | GCAGCTGCAGTTTGATGATGAAGACCTGGGGGCCTTGCTTGGCAA |
| | | CAGCACAGACCCAGCTGTGTTCACAGACCTGGCATCCGTCGACAA |
| | | CTCCGAGTTTCAGCAGCTGCTGAACCAGGGCATACCTGTGGCCCCC |
| | | CACACAACTGAGCCCATGCTGATGGAGTACCCTGAGGCTATAACT |
| | | CGCCTAGTGACAGGGGCCCAGAGGCCCCCCGACCCAGCTCCTGCT |
| | | CCACTGGGGGCCCCGGGGCTCCCCAATGGCCTCCTTTCAGGAGAT |
| | | GAAGACTTCTCCTCCATTGCGGACATGGACTTCTCAGCCCTGCTGA |
| | | GTCAGATCAGCTCCcaattgtgcgtacgcggatcctctgctggagacatgagagctgccaaccttt |
| | | ggccaagcccgctcatgatcaaacgctctaagaagaacagcctggccttgtccctgacggccgacca |
| | | gatggtcagtgccttgttggatgctgagccccccatactctattccgagtatgatcctaccagaccc |
| | | ttcagtgaagcttcgatgatgggcttactgaccaacctggcagacagggagATCGTTCACATGATCA |
| | | ACTGGGCGAAGAGGGTGCCAGGCTTTGTGGATTTGACCCTCCATGATCAGGT |
| | | CCACCTTCTAGAATGTGCCTGGCTAGAGATCCTGATGATTGGTGTG |
| | | GTCTGGCGCTCCATGGAGCACCCAGTGAAGCTACTGTTTGCTCCTA |
| | | ACTTGCTCTTGGACAGGGACCAGGGAAAATGTGTAGAGGGCCTGG |
| | | TGGAGATCTTCGACATGCTGCTGGCTACATCATCTCGGTTCCGCAT |
| | | GATGAATCTGCAGGGAGAGGAGTTTGTGTGCCTCAAATCTATTATT |
| | | TTGCTTAATTCTGGAGTGTACACATTTCTGCCCAGCACCCTGAAGT |
| | | CTCTGGAAGAGAAGGACCATATCCACCGAGTCCTGGACAAGATCA |
| | | CAGACACTTTGATCCACCTGATGGCCAAGGCAGGCCTGACCCTGC |
| | | AGCAGCAGCACCAGCGGCTGGCCCAGCTCCTCCTCATCCTCTCCCA |
| | | CATCAGGCACGCCAGTAACAAAGGCATGGAGCTGctgtacagcatgaagtgc |
| | | aagaacgtggtgcccctctatGaCctgctgctggaggcggcggacgcccaccgcctacatgcgccca |
| | | ctagccgtggaggggcatccgtggaggagacggaccaaagccacttggccactgcgggctctacttca |
| | | tcgcattccttgcaaaagtattacatcacgggggaggcagagggtttccctgccacaTaAGTCGACA |
| | | ATCAACCTCtggattacaaaatttgtgaaagattgactggtattcttaactatgttgctccttttacg |
| | | ctatgtggatacgctgctttaatgcctttgtatcatgctattgcttcccgtatggctttcattttctc |
| | | ctccttgtataaatcctggttgctgtctctttatgaggagttgtggcccgttgtcaggcaacgtggc |
| | | gtggtgtgcactgtgtttgctgacgcaacccccactggttggggcattgccaccacctgtcagctcct |
| | | ttccgggactttcgctttccccctccctattgccacggcggaactcatcgccgcctgccttgcccgct |
| | | gctggacaggggctcggctgttgggcactgacaattccgtggtgttgtcggggaaatcatcgtccttt |
| | | ccttggctgctcgcctgtgttgccacctggattctgcgcgggacgtccttctgctacgtcccttcgg |
| | | ccctcaatccagcggaccttccttcccgcggcctgctgccggctctgcggcctcttccgcgtctacg |
| | | ccttcgccctcagacgagtcggatctccctttgggccgcctccccgcgatatcagtggtccaggctct |
| | | agttttgactcaacaatatcaccagctgaagcctatagagtacgagccatagataaaataaaagattt |
| | | tatttagtctccagaaaaaggggggaatgaaagaccccacctgtaggtttggcaagctagcaataaa |
| | | agagcccacaacccctcactcggggcgccagtcctccgattgactgagtcgcccggccgcttcgagc |
| | | agacatgataagatacattgatgagtttggacaaaccacaactagaatgcagtgaaaaaaatgcttta |
| | | tttgtgaaatttgtgatgctattgctttatttgtaaccattataagctgcaataaacaagttaacaa |
| | | caacaattgcattcattttatgtttcaggttcagggggagatgtgggaggttttttaaagcaagta |
| | | aaacctctacaaatgtggtaaaatcgataaggatcgggtacccgtgtatccaataaaccctcttgca |
| | | gttgcatccgacttgtggtctcgctgttccttgggagggtctcctctgagtgattgactacccgtca |
| | | gcgggggtctttcacacatgcagcatgtatcaaaattaatttggttttttttcttaagctgtgccttc |
| | | tagttgccagccatctgttgtttgcccctcccccgtgccttccttgaccctggaaggtgccactccc |
| | | actgtcctttcctaataaaatgaggaaattgcatcgcattgtctgagtaggtgtcattctattctggg |
| | | gggtggggggggcaggacagcaagggggaggattgggaagacaatagcaggcatgctggggatgcgg |
| | | tgggctctatggagatcccgcggtacctcgcgaatgcatctagatccaatggcctttttggcccagac |
| | | atgataagatacattgatgagtttggacaaaccacaactagaatgcagtgaaaaaaatgctttattt |
| | | gtgaaatttgtgatgctattgctttatttgtaaccattataagctgcaataaacaagttgcggccgc |
| | | ttagccctcccacacataaccagagggcagcaattcacgaatcccaactgccgtcggctgtccatca |
| | | ctgtccttcactatggctttgatcccaggatgcagatcgagaagcacctgtcggcaccgtccgcaggg |
| | | gctcaagatgcccctgttctcatttccgatcgcgacgatacaagtcaggttgccagctgccgcagca |
| | | gcagcagtgcccagcaccacgagttctgcacaaggtcccccagtaaaatgatatacattgacaccag |
| | | tgaagatgcggccgtcgctagagagagctgcgctggcgacgctgtagtcttcagagatggggatgct |
| | | gttgattgtagccgttgctctttcaatgagggtggattcttcttgagacaaaggcttggccatgcgg |
| | | ccgccgctcggtgttcgaggccacacgcgtcaccttaatatgcgaagtggacctcggaccgcgccgc |
| | | cccgactgcatctgcgtgttcgaattcgccaatgacaagacgctgggggggtttgtgtcatcatagaa |
| | | ctaaagacatgcaaatatatttcttccggggggtaccggcctttttggccATTGGatcggatctgg |
| | | ccaaaaaggcccttaagtatttacattaaatggccatagtacttaaagttacattggcttccttgaaa |
| | | taaacatggagtattcagaatgtgtcataaatatttctaattttaagatagtatctccattggcttt |
| | | ctactttttcttttatttttttttgtcctctgtcttccatttgttgttgttgttgtttgtttgttt |
| | | gtttgttggttggttggttaatttttttttaaagatcctacactatagttcaagctagactattagc |
| | | tactctgtaacccagggtgaccttgaagtcatgggtagcctgctgttttagccttcccacatctaag |
| | | attacaggtatgagctatcatttttggtatattgattgattgattgattgatgtgtgtgtgtgtgat |
| | | tgtgtttgtgtgtgtgaTtgtgTaTatgtgtgtatggTtgtgtgtgaTtgtgtgtatgtatgTTtgt |
| | | gtgtgaTtgTgtgtgtgtgaTtgtgcatgtgtgtgtgtgtgaTtgtgtTtatgtgtatgaTtgtgtgt |
| | | gtgtgtgtgtgtgtgtgtgtgtgtgtgtgtgtgtgtgtgttgtgTaTaTatatttatggtagtgagag |
| | | Gcaacgctccggctcaggtgtcaggttggtttttgagacagagtctttcacttagcttggaattcact |
| | | ggccgtcgttttacaacgtcgtgactgggaaaaccctggcgttacccaacttaatcgccttgcagca |
| | | catccccctttcgccagctggcgtaatagcgaagaggcccgcaccgatcgcccttcccaacagttgcg |
| | | cagcctgaatggcgaatggcgcctgatgcggtattttctccttacgcatctgtgcggtatttcaca |
| | | ccgcatatggtgcactctcagtacaatctgctctgatgccgcatagttaagccagccccgacacccg |
| | | ccaacacccgctgacgcgccctgacgggcttgtctgctcccggcatccgcttacagacaagctgtga |
| | | ccgtctccgggagctgcatgtgtcagaggttttcaccgtcatcaccgaaacgcgcgagacgaaaggg |
| | | cctcgtgatacgcctatttttataggttaatgtcatgataataatggtttcttagacgtcaggtggc |
| | | acttttcggggaaatgtgcgcggaacccctatttgtttatttttctaaatacattcaaatatgtatc |
| | | cgctcatgagacaataaccctgataaatgcttcaataatattgaaaaaggaagagtatgagtattca |
| | | acatttccgtgtcgcccttattcccttttttgcggcattttgccttcctgtttttgctcacccaga |
| | | aacgctggtgaaagtaaaagatgctgaagatcagttgggtgcacgagtgggttacatcgaactggatc |
| | | tcaacagcggtaagatccttgagagttttcgccccgaagaacgttttccaatgatgagcacttttaa |
| | | agttctgctatgtggcgcggtattatcccgtattgacgccgggcaagagcaactcggtcgccgcata |
| | | cactattctcagaatgacttggttgagtactcaccagtcacagaaaagcatcttacggatggcatga |
| | | cagtaagagaattatgcagtgctgccataaccatgagtgataacactgcggccaacttacttctgac |
| | | aacgatcggaggaccgaaggagctaaccgcttttttgcacaacatgggggatcatgtaactcgcctt |
| | | gatcgttgggaaccggagctgaatgaagccataccaaacgacgagcgtgacaccacgatgcctgtagc |
| | | aatggcaacaacgttgcgcaaactattaactggcgaactacttactctagcttcccggcaacaattaa |
| | | tagactggatggaggcggataaagttgcaggaccacttctgcgctcggcccttccggctggctggttt |
| | | attgctgataaatctggagccggtgagcgtgggtctcgcggtatcattgcagcactggggccagatg |
| | | gtaagccctcccgtatcgtagttatctacacgacggggagtcaggcaactatggatgaacgaaataga |
| | | cagatcgctgagataggtgcctcactgattaagcattggtaactgtcagaccaagtttactcatatat |
| | | actttagattgatttaaaacttcatttttaatttaaaaggatctaggtgaagatcctttttgataat |
| | | ctcatgaccaaaatcccttaacgtgagttttcgttccactgagcgtcagaccccgtagaaaagatcaa |
| | | aggatcttcttgagatcctttttttctgcgcgtaatctgctgcttgcaaacaaaaaaaccaccgctac |
| | | cagcggtggtttgtttgccggatcaagagctaccaactctttttccgaaggtaactggcttcagcaga |
| | | gcgcagataccaaatactgtccttctagtgtagccgtagttaggccaccacttcaagaactctgtagc |
| | | accgcctacatacctcgctctgctaatcctgttaccagtggctgctgccagtggcgataagtcgtgt |
| | | cttaccgggttggactcaagacgatagttaccggataaggcgcagcggtcgggctgaacggggggtt |
| | | cgtgcacacagcccagcttggagcgaacgacctacaccgaactgagatacctacagcgtgagctatg |
| | | agaaagcgccacgcttcccgaagggagaaaggcggacaggtatccggtaagcggcagggtcggaaca |
| | | ggagagcgcacgagggagcttccagggggaaacgcctggtatctttatagtcctgtcgggtttcgcc |
| | | acctctgacttgagcgtcgatttttgtgatgctcgtcaggggggggagcctatggaaaaacgccagc |
| | | aacgcggcctttttacggttcctggccttttgctggccttttgctcacatgttctttcctgcgttat |
| | | cccctgattctgtggataaccgtattaccgcctttgagtgagctgataccgctcgccgcagccgaac |
| | | gaccgagcgcagcgagtcagtgagcgaggaagcggaagagcgcccaatacgcaaaccgcctctcccc |
| | | gcgcgttggccgattcattaatgcagctggcacgacaggtttcccgactggaaagcgggcagtgagc |
| | | gcaacgcaattaatgtgagttagctcactcattaggcaccccaggctttacactttatgcttccggc |
| | | tcgtatgttgtgtggaattgtgagcggataacaatttcacacaggaaacagctatgaccatgattac |
| | | gcc |
| SB07139 vector | 81 | aagcttgaattcgagcttgcatgcctgcaggtcgttacataacttacggtaaatggcccgcctggctg |
| | | accgcccaacgacccccgcccattgacgtcaataatgacgtatgttcccatagtaacgccaataggga |
| | | ctttccattgacgtcaatgggtggagtatttacggtaaactgcccacttggcagtacatcaagtgtat |
| | | catatgccaagtacgccccctattgacgtcaatgacggtaaatggcccgcctggcattatgcccagt |
| | | acatgaccttatgggactttcctacttggcagtacatctacgtattagtcatcgctattaccatggt |
| | | gatgcggttttggcagtacatcaatgggcgtggatagcggtttgactcacggggatttccaagtctcc |
| | | accccattgacgtcaatgggagtttgttttggcaccaaaatcaacgggactttccaaaatgtcgtaa |
| | | caactccgccccattgacgcaaatgggcggtaggcgtgtacggtgggaggtctatataagcagagc |
| | | tcaataaaagagcccacaacccctcactcggcgcgccagtcctccgattgactgagtcgcccgggta |
| | | cccgtgtatccaataaaccctcttgcagttgcatccgacttgtggtctcgctgttccttgggagggtc |
| | | tcctctgagtgattgactacccgtcagcgggggtctttcatttgggggctcgtccgagatcgggagac |
| | | ccctgcccagggaccaccgacccaccaccgggaggtaagctggccagcaacttatctgtgtctgtcc |
| | | gattgtctagtgtctatgactgattttatgcgcctgcgtcggtactagttagctaactagctctgtat |
| | | ctggcggacccgtggtggaactgacgagttcggaacacccggccgcaaccctgggagacgtcccagg |
| | | gacttcgggggccgtttttgtggcccgacctgagtcctaaaatcccgatcgtttaggactctttggt |
| | | gcaccccccttagaggagggatatgtggttctggtaggagacgagaacctaaaacagttcccgcct |
| | | ccgtctgaatttttgctttcggtttgggaccgaagccgcgccgcgcgtcttgtctgctgcagcatcg |
| | | ttctgtgttgtctctgtctgactgtgtttctgtatttgtctgaaaatatgggccccccctcgagtccc |
| | | cagcatgcctgctattctcttcccaatcctcccccttgctgtcctgccccaccccacccccca |
| | | gaatagaatgacacctactcagacaatgcgatgcaatttcctcattttattaggaaaggacagtgg |
| | | gagtggcaccttccagggtcaaggaaggcacgggggaggggcaaacaacagatggctggcaactaga |
| | | aggcacagttacttaCACAGGCCGCACAGATTCTCTCCGCAGCCGTTCGTTTCTTCTCCG |
| | | CTCTCTGCACCGAGGGGCGAAGCAGTAGGTCAGGCAACAGATCAC |
| | | GAAGATGCCGTTCACGGAGATCAGTGTGATGGCCCAGCTTGGCAG |
| | | CAGTTGAAGAGATCCACCGCCACTGCCACCGCCGCCACTACCGCC |
| | | ACCAAAGAAGCTGGAGATTGTAGACACGGCGAGTCCCTGTGTAAT |
| | | TCCAGATCCTCCGCCTCCGCTACCACCTCCGCCGCTAGAGGCGTTC |
| | | AGGTAGCTCATCACTCTGTCGATGGTCACGGCTCTGATCCGGAAGG |
| | | CGTGCAGCAGGATGCACAGCTTGATCTTGGTCTTGTAGAAGTCGG |
| | | GTTCTTCCAGGCTAGACTTCTGGGGCACTGTCTCGCTGTTGAAGTT |
| | | CAGGGCCTGCATCAGCTCGTCGATCACGGCCAGCATATTCTGGTCC |
| | | AGGAAGATCTGCCGCTTGGGGTCCATCAGCAGCTTGGCGTTCATG |
| | | GTCTTGAATTCCACCTGGTACATCTTCAGGTCCTCGTAGATGCTGC |
| | | TCAGGCACAGGGCCATCATGAAGGAGGTCTTTCTGCTGGCCAGGC |
| | | AAGAGCCGTTGGTGATGAAGCTGGTTTCCCGGCTGTTCAGGCAGCT |
| | | CTCGTTCTTGGTCAGTTCCAGAGGCAGGCAGGCTTCCACGGTGCTG |
| | | GTCTTATCCTTGGTGATGTCCTCGTGGTCGATTTCCTCGCTGGTGCA |
| | | GGGGTAGAATTCCAGGGTCTGTCTGGCCTTCTGCAGCATGTTGGAC |
| | | ACGGCTCTCAGCAGGTTCTGGCTGTGGTGCAGACAAGGGAACATG |
| | | CCAGGATCAGGAGTGGCCACAGGCAGGTTTCTAGATCCGCCGCCA |
| | | GATCCACCACCTGATCCGCCACCGCTTCCTCCGCCAGAACATGGCA |
| | | CGCTGGCCCATTCGCTCCAAGAGCTGCTGTAGTACCGGTCCTGGGC |
| | | TCTGACGCTGATGCTGGCGTTCTTTCTGCAGATCACGGTGGCGCTG |
| | | GTCTTGTCGGTGAACACCCGGTCCTTTTTCTCGCGCTTGGACTTGCC |
| | | CTGCACTTGCACGCAAAAGGTCAGGCTGAAGTAGCTGTGGGGTGT |
| | | AGACCAGGTGTCGGGGTACTCCCAGGACACTTCCACCTGTCTGCTG |
| | | TTCTTCAGAGGCTTCAGCTGCAGGTTCTTTGGAGGATCGGGCTTGA |
| | | TGATGTCCCGGATGAAAAAGCTGGAGGTGTAGTTCTCGTACTTCAG |
| | | CTTGTGCACGGCGTCCACCATCACTTCGATAGGCAGAGACTCTTCG |
| | | GCGGCTGGACAGGCGCTGTCCTCTTGGCATTCCACGCTGTACTCGT |
| | | ATTCTTTGTTGTCGCCCCGCACTCTTTCGGCAGACAGTGTAGCGGC |
| | | GCCACATGTAACGCCCTGAGGATCACTGCTGCCTCTGCTGGACTTC |
| | | ACGCTGAAGGTCAGGTCGGTGCTGATGGTGGTCAGCCACCAACAT |
| | | GTGAACCGGCCGCTGTAGTTCTTGGCCTCGCATCTCAGGAAGGTCT |
| | | TGTTCTTGGGCTCTTTCTGGTCCTTCAGGATGTCGGTGCTCCAAATG |
| | | CCATCCTCTTTCTTGTGGAGCAGCAGCAGGCTGTGGCTCAGCACTT |
| | | CTCCGCCTTTGTGACAGGTGTACTGGCCGGCGTCGCCAAACTCTTT |
| | | CACTTGGATGGTCAGGGTCTTGCCGCTGCCGAGCACCTCGCTAGAC |
| | | TGATCCAGTGTCCAGGTGATGCCGTCCTCTTCAGGGGTATCGCAGG |
| | | TCAGCACCACCATCTCGCCAGGAGCATCGGGATACCAGTCCAGTT |
| | | CCACCACGTACACGTCTTTCTTCAGCTCCCAGATGGCCACCAGAGG |
| | | AGAGGCCAGGAACACCAGGCTGAACCAGCTGATGACCAGCTGCTG |
| | | GTGACACATCATGGTGGCGACACCGGTACGCGTTGGCCCCCATTAT |
| | | ATACCCTCTAGAACTAGTtatccactccgtgtaagggagagtgagcctcttacgaatcTTC |
| | | GGCGTCGACTGCTTCattccgaggcgactgataccTTCGGCGTCGACTGCTTCat |
| | | acgaaggcagtccgattcTTCGGCGTCGACTGCTTCaacctttactgagacgggacTTCG |
| | | GCGTCGACTGCTTCaaaggcgttgcgaatcctcatgcgattgttacgaaacccgTTAATTA |
| | | AAGAGCGAGATTCCGTCTCAAAGAAAAAAAAAGTAATGAAATGA |
| | | ATAAAATGAGTCCTAGAGCCAGTAAATGTCGTAAATGTCTCAGCT |
| | | AGTCAGGTAGTAAAAGGTCTCAACTAGGCAGTGGCAGAGCAGGAT |
| | | TCAAATTCAGGGCTGTTGTGATGCCTCCGCAGACTCTGAGCGCCAC |
| | | CTGGTGGTAATTTGTCTGTGCCTCTTCTGACGTGGAAGAACAGCAA |
| | | CTAACACACTAACACGGCATTTACTATGGGCCAGCCATTGTCCATC |
| | | TAGATGGccgataaaataaaagattttatttagtctccagaaaaaggggggaatgaaagaccccacc |
| | | tgtaggtttggcaagctagctgcaGTGTGTCAGTTAGGGTGTGGAAAGTCCCCAGG |
| | | CTCCCCAGCAGGCAGAAGTATGCAAAGCATGCATCTCAATTAGTC |
| | | AGCAACCAGGTGTGGAAAGTCCCCAGGCTCCCCAGCAGGCAGAAG |
| | | TATGCAAAGCATGCATCTCAATTAGTCAGCAACCATAGTCCCGCCC |
| | | CTAACTCCGCCCATCCCGCCCCTAACTCCGCCCAGTTCCGCCCATT |
| | | CTCCGCCCCATGGCTGACTAATTTTTTTTATTTATGCAGAGGCCGA |
| | | GGCCGCCTCTGCCTCTGAGCTATTCCAGAAGTAGTGAGGAGGCTTT |
| | | TTTGGAGGCCTAGGCTTTTGCAAAGGATCCGCCACCATGTCTAGAC |
| | | CTGGCGAGAGGCCCTTCCAGTGCCGGATCTGCATGCGGAACTTCA |
| | | GCAACATGAGCAACCTGACCAGACACACCCGGACACACACAGGC |
| | | GAGAAGCCTTTTCAGTGCAGAATCTGTATGCGCAATTTCTCCGACA |
| | | GAAGCGTGCTGCGGAGACACCTGAGAACCCACACCGGCAGCCAG |
| | | AAACCATTCCAGTGTCGCATCTGTATGAGAAACTTTAGCGACCCCT |
| | | CCAATCTGGCCCGGCACACCAGAACACATACCGGGGAAAAACCCT |
| | | TTCAGTGTAGGATATGCATGAGGAATTTTTCCGACCGGTCCAGCCT |
| | | GAGGCGGCACCTGAGGACACATACTGGCTCCCAAAAGCCGTTCCA |
| | | ATGTCGGATATGTATGCGCAACTTTAGCCAGAGCGGCACCCTGCA |
| | | CAGACACACAAGAACCCATACTGGCGAGAAACCTTTCCAATGTAG |
| | | AATCTGCATGCGAAATTTTTCCCAGCGGCCTAATCTGACCAGGCAT |
| | | CTGAGGACCCACCTGAGAGGATCTaCCTGCAGGGATGAGTTTCCCA |
| | | CCATGGTGTTTCCTTCTGGGCAGATCAGCCAGGCCTCGGCCTTGGC |
| | | CCCGGCCCCTCCCCAAGTCCTGCCCCAGGCTCCAGCCCCTGCCCCT |
| | | GCTCCAGCCATGGTATCAGCTCTGGCCCAGGCCCCAGCCCCTGTCC |
| | | CAGTCCTAGCCCCAGGCCCTCCTCAGGCTGTGGCCCCACCTGCCCC |
| | | CAAGCCCACCCAGGCTGGGGAAGGAACGCTGTCAGAGGCCCTGCT |
| | | GCAGCTGCAGTTTGATGATGAAGACCTGGGGGCCTTGCTTGGCAA |
| | | CAGCACAGACCCAGCTGTGTTCACAGACCTGGCATCCGTCGACAA |
| | | CTCCGAGTTTCAGCAGCTGCTGAACCAGGGCATACCTGTGGCCCCC |
| | | CACACAACTGAGCCCATGCTGATGGAGTACCCTGAGGCTATAACT |
| | | CGCCTAGTGACAGGGGCCCAGAGGCCCCCCGACCCAGCTCCTGCT |
| | | CCACTGGGGGCCCCGGGGCTCCCCAATGGCCTCCTTTCAGGAGAT |
| | | GAAGACTTCTCCTCCATTGCGGACATGGACTTCTCAGCCCTGCTGA |
| | | GTCAGATCAGCTCCcaattgtgcgtacgcggatcctctgctggagacatgagagctgccaaccttt |
| | | ggccaagcccgctcatgatcaaacgctctaagaagaacagcctggccttgtccctgacggccgaccag |
| | | atggtcagtgccttgttggatgctgagccccccatactctattccgagtatgatcctaccagaccctt |
| | | cagtgaagcttcgatgatgggcttactgaccaacctggcagacagggagCTGGTTCACATGATCAACT |
| | | GGGCGAAGAGGGTGCCAGGCTTTGTGGATTTGACCCTCCATGATCAGGT |
| | | CCACCTTCTAGAATGTGCCTGGCTAGAGATCCTGATGATTGGTCTC |
| | | GTCTGGCGCTCCATGGAGCACCCAGTGAAGCTACTGTTTGCTCCTA |
| | | ACTTGGTGTTGGACAGGGACGAGGGAAAATGTGTAGAGGGCCTGG |
| | | TGGAGATCTTCGACATGCTGCTGGCTACATCATCTCGGTTCCGCAT |
| | | GATGAATCTGCAGGGAGAGGAGTTTGTGTGCCTCAAATCTATTATT |
| | | TTGCTTAATTCTGGAGTGTACACATTTCTGCCCAGCACCCTGAAGT |
| | | CTCTGGAAGAGAAGGACCATATCCACCGAGTCCTGGACAAGATCA |
| | | CAGACACTTTGATCCACCTGATGGCCAAGGCAGGCCTGACCCTGC |
| | | AGCAGCAGCACCAGCGGCTGGCCCAGCTCCTCCTCATCCTCTCCCA |
| | | CATCAGGCACATGAGTAACAAAGGCATGGAGCTGctgtacagcatgaagtgc |
| | | aagaacgtggtgcccctctatGaCctgctgctggaggcggcggacgcccaccgcctacatgcgcccac |
| | | tagccgtggaggggcatccgtggaggagacggaccaaagccacttggccactgcgggctctacttca |
| | | tcgcattccttgcaaaagtattacatcacgggggaggcagagggtttccctgccacaTaAGTCGACAA |
| | | TCAACCTCtggattacaaaatttgtgaaagattgactggtattcttaactatgttgctccttttac |
| | | gctatgtggatacgctgctttaatgcctttgtatcatgctattgcttcccgtatggctttcattttct |
| | | cctccttgtataaatcctggttgctgtctctttatgaggagttgtggcccgttgtcaggcaacgtgg |
| | | cgtggtgtgcactgtgtttgctgacgcaacccccactggttggggcattgccaccacctgtcagctc |
| | | ctttccgggactttcgctttccccctccctattgccacggcggaactcatcgccgcctgccttgcccg |
| | | ctgctggacaggggctcggctgttgggcactgacaattccgtggtgttgtcggggaaatcatcgtcct |
| | | ttccttggctgctcgcctgtgttgccacctggattctgcgcgggacgtccttctgctacgtcccttcg |
| | | gccctcaatccagcggaccttccttcccgcggcctgctgccggctctgcggcctcttccgcgtctacg |
| | | ccttcgccctcagacgagtcggatctccctttgggccgcctccccgcgatatcagtggtccaggctct |
| | | agttttgactcaacaatatcaccagctgaagcctatagagtacgagccatagataaaataaaagatt |
| | | ttatttagtctccagaaaaaggggggaatgaaagaccccacctgtaggtttggcaagctagcaataa |
| | | aagagcccacaacccctcactcggggcgccagtcctccgattgactgagtcgcccggccgcttcgagc |
| | | agacatgataagatacattgatgagtttggacaaaccacaactagaatgcagtgaaaaaaatgcttta |
| | | tttgtgaaatttgtgatgctattgctttatttgtaaccattataagctgcaataaacaagttaacaac |
| | | aacaattgcattcattttatgtttcaggttcagggggagatgtgggaggttttttaaagcaagtaaaa |
| | | cctctacaaatgtggtaaaatcgataaggatcgggtacccgtgtatccaataaaccctcttgcagtt |
| | | gcatccgacttgtggtctcgctgttccttggggggtctcctctgagtgattgactacccgtcagcgg |
| | | gggtctttcacacatgcagcatgtatcaaaattaatttggttttttttcttaagctgtgccttctag |
| | | ttgccagccatctgttgtttgcccctcccccgtgccttccttgaccctggaaggtgccactcccactg |
| | | tcctttcctaataaaatgaggaaattgcatcgcattgtctgagtaggtgtcattctattctgggggg |
| | | tggggggggcaggacagcaagggggaggattgggaagacaatagcaggcatgctggggatgcggtgg |
| | | gctctatggagatcccgcggtacctcgcgaatgcatctagatccaatggcctttttggcccagaca |
| | | tgataagatacattgatgagtttggacaaaccacaactagaatgcagtgaaaaaaatgctttatttgt |
| | | gaaatttgtgatgctattgctttatttgtaaccattataagctgcaataaacaagttgcggccgctta |
| | | gccctcccacacataaccagagggcagcaattcacgaatcccaactgccgtcggctgtccatcactgt |
| | | ccttcactatggctttgatcccaggatgcagatcgagaagcacctgtcggcaccgtccgcaggggct |
| | | caagatgcccctgttctcatttccgatcgcgacgatacaagtcaggttgccagctgccgcagcagcag |
| | | cagtgcccagcaccacgagttctgcacaaggtcccccagtaaaatgatatacattgacaccagtgaag |
| | | atgcggccgtcgctagagagagctgcgctggcgacgctgtagtcttcagagatggggatgctgttga |
| | | ttgtagccgttgctctttcaatgagggtggattcttcttgagacaaaggcttggccatgcggccgcc |
| | | gctcggtgttcgaggccacacgcgtcaccttaatatgcgaagtggacctcggaccgcgccgccccgac |
| | | tgcatctgcgtgttcgaattcgccaatgacaagacgctgggcggggtttgtgtcatcatagaactaa |
| | | agacatgcaaatatatttcttccggggggtaccggcctttttggccATTGGatcggatctggccaaa |
| | | aaggcccttaagtatttacattaaatggccatagtacttaaagttacattggcttccttgaaataaac |
| | | atggagtattcagaatgtgtcataaatatttctaattttaagatagtatctccattggctttctact |
| | | ttttttttatttttttttgtcctctgtcttccatttgttgttgttgttgtttgtttgtttgtttgtt |
| | | ggttggttggttaatttttttttaaagatcctacactatagttcaagctagactattagctactctg |
| | | taacccagggtgaccttgaagtcatgggtagcctgctgttttagccttcccacatctaagattacag |
| | | gtatgagctatcatttttggtatattgattgattgattgattgatgtgtgtgtgtgtgattgtgttt |
| | | gtgtgtgtgaTtgtgTaTatgtgtgtatggTtgtgtgtgaTtgtgtgtatgtatgTTtgtgtgtgaT |
| | | tgTgtgtgtgtgaTtgtgcatgtgtgtgtgtgtgaTtgtgtTtatgtgtatgaTtgtgtgtgtgtgt |
| | | gtgtgtgtgtgtgtgtgtgtgtgtgtgtgtgtgttgtgTaTaTatatttatggtagtgagagGcaa |
| | | cgctccggctcaggtgtcaggttggtttttgagacagagtctttcacttagcttggaattcact |
| | | ggccgtcgttttacaacgtcgtgactgggaaaaccctggcgttacccaacttaatcgccttgcagca |
| | | catccccctttcgccagctggcgtaatagcgaagaggcccgcaccgatcgcccttcccaacagttgc |
| | | gcagcctgaatggcgaatggcgcctgatgcggtattttctccttacgcatctgtgcggtatttcacac |
| | | cgcatatggtgcactctcagtacaatctgctctgatgccgcatagttaagccagccccgacacccgcc |
| | | aacacccgctgacgcgccctgacgggcttgtctgctcccggcatccgcttacagacaagctgtgacc |
| | | gtctccgggagctgcatgtgtcagaggttttcaccgtcatcaccgaaacgcgcgagacgaaagggcc |
| | | tcgtgatacgcctatttttataggttaatgtcatgataataatggtttcttagacgtcaggtggcact |
| | | tttcggggaaatgtgcgcggaacccctatttgtttatttttctaaatacattcaaatatgtatccgct |
| | | catgagacaataaccctgataaatgcttcaataatattgaaaaaggaagagtatgagtattcaacat |
| | | ttccgtgtcgcccttattcccttttttgcggcattttgccttcctgtttttgctcacccagaaacgc |
| | | tggtgaaagtaaaagatgctgaagatcagttgggtgcacgagtgggttacatcgaactggatctcaac |
| | | agcggtaagatccttgagagttttcgccccgaagaacgttttccaatgatgagcacttttaaagtt |
| | | ctgctatgtggcgcggtattatcccgtattgacgccgggcaagagcaactcggtcgccgcatacac |
| | | tattctcagaatgacttggttgagtactcaccagtcacagaaaagcatcttacggatggcatgacagt |
| | | aagagaattatgcagtgctgccataaccatgagtgataacactgcggccaacttacttctgacaacg |
| | | atcggaggaccgaaggagctaaccgcttttttgcacaacatgggggatcatgtaactcgccttgatc |
| | | gttgggaaccggagctgaatgaagccataccaaacgacgagcgtgacaccacgatgcctgtagcaat |
| | | ggcaacaacgttgcgcaaactattaactggcgaactacttactctagcttcccggcaacaattaatag |
| | | actggatggaggcggataaagttgcaggaccacttctgcgctcggcccttccggctggctggtttat |
| | | tgctgataaatctggagccggtgagcgtgggtctcgcggtatcattgcagcactggggccagatggta |
| | | agccctcccgtatcgtagttatctacacgacggggagtcaggcaactatggatgaacgaaatagaca |
| | | gatcgctgagataggtgcctcactgattaagcattggtaactgtcagaccaagtttactcatatat |
| | | actttagattgatttaaaacttcatttttaatttaaaaggatctaggtgaagatcctttttgataat |
| | | ctcatgaccaaaatcccttaacgtgagttttcgttccactgagcgtcagaccccgtagaaaagatcaa |
| | | aggatcttcttgagatcctttttttctgcgcgtaatctgctgcttgcaaacaaaaaaaccaccgcta |
| | | ccagcggtggtttgtttgccggatcaagagctaccaactctttttccgaaggtaactggcttcagca |
| | | gagcgcagataccaaatactgtccttctagtgtagccgtagttaggccaccacttcaagaactctgt |
| | | agcaccgcctacatacctcgctctgctaatcctgttaccagtggctgctgccagtggcgataag |
| | | tcgtgtcttaccgggttggactcaagacgatagttaccggataaggcgcagcggtcgggctgaacg |
| | | gggggttcgtgcacacagcccagcttggagcgaacgacctacaccgaactgagatacctacagcg |
| | | tgagctatgagaaagcgccacgcttcccgaagggagaaaggcggacaggtatccggtaagcggcag |
| | | ggtcggaacaggagagcgcacgagggagcttccagggggaaacgcctggtatctttatagtcctgtcg |
| | | ggtttcgccacctctgacttgagcgtcgatttttgtgatgctcgtcaggggggcggagcctatggaaa |
| | | aacgccagcaacgcggcctttttacggttcctggccttttgctggccttttgctcacatgttctttcc |
| | | tgcgttatcccctgattctgtggataaccgtattaccgcctttgagtgagctgataccgctcgccgca |
| | | gccgaacgaccgagcgcagcgagtcagtgagcgaggaagcggaagagcgcccaatacgcaaaccgc |
| | | ctctccccgcgcgttggccgattcattaatgcagctggcacgacaggtttcccgactggaaagcgggc |
| | | agtgagcgcaacgcaattaatgtgagttagctcactcattaggcaccccaggctttacactttatgct |
| | | tccggctcgtatgttgtgtggaattgtgagcggataacaatttcacacaggaaacagctatgacca |
| | | tgattacgcc |
| WRPW motif | 156 | WRPW |
| IL-12 | 184 | ATGTGTCACCAGCAGCTGGTCATCAGCTGGTTCAGCCTGGTGTTCC |
| | | TGGCCTCTCCTCTGGTGGCCATCTGGGAGCTGAAGAAAGACGTGT |
| | | ACGTGGTGGAACTGGACTGGTATCCCGATGCTCCTGGCGAGATGG |
| | | TGGTGCTGACCTGCGATACCCCTGAAGAGGACGGCATCACCTGGA |
| | | CACTGGATCAGTCTAGCGAGGTGCTCGGCAGCGGCAAGACCCTGA |
| | | CCATCCAAGTGAAAGAGTTTGGCGACGCCGGCCAGTACACCTGTC |
| | | ACAAAGGCGGAGAAGTGCTGAGCCACAGCCTGCTGCTGCTCCACA |
| | | AGAAAGAGGATGGCATTTGGAGCACCGACATCCTGAAGGACCAGA |
| | | AAGAGCCCAAGAACAAGACCTTCCTGAGATGCGAGGCCAAGAACT |
| | | ACAGCGGCCGGTTCACATGTTGGTGGCTGACCACCATCAGCACCG |
| | | ACCTGACCTTCAGCGTGAAGTCCAGCAGAGGCAGCAGTGATCCTC |
| | | AGGGCGTTACATGTGGCGCCGCTACACTGTCTGCCGAAAGAGTGC |
| | | GGGGCGACAACAAAGAATACGAGTACAGCGTGGAATGCCAAGAG |
| | | GACAGCGCCTGTCCAGCCGCCGAAGAGTCTCTGCCTATCGAAGTG |
| | | ATGGTGGACGCCGTGCACAAGCTGAAGTACGAGAACTACACCTCC |
| | | AGCTTTTTCATCCGGGACATCATCAAGCCCGATCCTCCAAAGAACC |
| | | TGCAGCTGAAGCCTCTGAAGAACAGCAGACAGGTGGAAGTGTCCT |
| | | GGGAGTACCCCGACACCTGGTCTACACCCCACAGCTACTTCAGCCT |
| | | GACCTTTTGCGTGCAAGTGCAGGGCAAGTCCAAGCGCGAGAAAAA |
| | | GGACCGGGTGTTCACCGACAAGACCAGCGCCACCGTGATCTGCAG |
| | | AAAGAACGCCAGCATCAGCGTCAGAGCCCAGGACCGGTACTACAG |
| | | CAGCTCTTGGAGCGAATGGGCCAGCGTGCCATGTTCTGGCGGAGG |
| | | AAGCGGTGGCGGATCAGGTGGTGGATCTGGCGGCGGATCTAGAAA |
| | | CCTGCCTGTGGCCACTCCTGATCCTGGCATGTTCCCTTGTCTGCACC |
| | | ACAGCCAGAACCTGCTGAGAGCCGTGTCCAACATGCTGCAGAAGG |
| | | CCAGACAGACCCTGGAATTCTACCCCTGCACCAGCGAGGAAATCG |
| | | ACCACGAGGACATCACCAAGGATAAGACCAGCACCGTGGAAGCCT |
| | | GCCTGCCTCTGGAACTGACCAAGAACGAGAGCTGCCTGAACAGCC |
| | | GGGAAACCAGCTTCATCACCAACGGCTCTTGCCTGGCCAGCAGAA |
| | | AGACCTCCTTCATGATGGCCCTGTGCCTGAGCAGCATCTACGAGGA |
| | | CCTGAAGATGTACCAGGTGGAATTCAAGACCATGAACGCCAAGCT |
| | | GCTGATGGACCCCAAGCGGCAGATCTTCCTGGACCAGAATATGCT |
| | | GGCCGTGATCGACGAGCTGATGCAGGCCCTGAACTTCAACAGCGA |
| | | GACAGTGCCCCAGAAGTCTAGCCTGGAAGAACCCGACTTCTACAA |
| | | GACCAAGATCAAGCTGTGCATCCTGCTGCACGCCTTCCGGATCAG |
| | | AGCCGTGACCATCGACAGAGTGATGAGCTACCTGAACGCCTCT |
| SB07351 | 185 | ATGgctggagacatgagagctgccaacctttggccaagcccgctcatgatcaaacgctctaagaagaa |
| ERT2*mut81- | | cagcctggccttgtccctgacggccgaccagatggtcagtgccttgttggatgctgagccccccata |
| Casp9-IRES- | | ctctattccgagtatgatcctaccagacccttcagtgaagcttcgatgatgggcttactgaccaac |
| RFP | | ctggcagacagggagCTGGTTCACATGATCAACTGGGCGAAGAGGGTGCCAGGCTTTGTGG |
| Nucleotide | | ATTTGACCCTCCATGATCAGGTCCACCTTCTAGAATGTGCCTGGAT |
| sequence | | GGAGATCCTGATGATTGGTGTGGTCTGGCGCTCCATGGAGCACCC |
| IRES-RFP in | | AGTGAAGCTACTGTTTGCTCCTAACTTGCTCTTGGACAGGGACCAG |
| bold italic | | GGAAAATGTGTAGAGGGCCTGGTGGAGATCTTCGACATGCTGCTG |
| | | GCTACATCATCTCGGTTCCGCATGATGAATCTGCAGGGAGAGGAG |
| | | TTTGTGTGCCTCAAATCTATTATTTTGCTTAATTCTGGAGTGTACAC |
| | | ATTTCTGCCCAGCACCCTGAAGTCTCTGGAAGAGAAGGACCATAT |
| | | CCACCGAGTCCTGGACAAGATCACAGACACTTTGATCCACCTGAT |
| | | GGCCAAGGCAGGCCTGACCCTGCAGCAGCAGCACCAGCGGCTGGC |
| | | CCAGCTCCTCCTCATCCTCTCCCACATCAGGCACATGAGTAACAAA |
| | | GGCATGGAGCTGctgtacagcatgaagtgcaagaacgtggtgcccctctatGaCctgctgctgga |
| | | ggcggcggacgcccaccgcctacatgcgcccactagccgtggaggggcatccgtggaggagacggacc |
| | | aaagccacttggccactgcgggctctacttcatcgcattccttgcaaaagtattacatcacgggggag |
| | | gcagagggtttccctgccacagggggtggaggttcagggggtggaggttcaggtggtggcggtagtgt |
| | | cgatggcttcgatgtcggtgctcttgagagtttgaggggaaatgcagatttggcttacatcctgag |
| | | catggagccctgtggccactgcctcattatcaacaatgtgaacttctgccgtgagtccgggctccgc |
| | | acccgcactggctccaacatcgactgtgagaagttgcggcgtcgcttctcctcgctgcatttcatgg |
| | | tggaggtgaagggcgacctgactgccaagaaaatggtgctggctttgctggagctggcgcggcagg |
| | | accacggtgctctggactgctgcgtggtggtcattctctctcacggctgtcaggccagccacctgca |
| | | gttcccaggggctgtctacggcacagatggatgccctgtgtcggtcgagaagattgtgaacatcttc |
| | | aatgggaccagctgccccagcctgggagggaagcccaagctctttttcatccaggcctgtggtgggg |
| | | agcagaaagaccatgggtttgaggtggcctccacttcccctgaagacgagtcccctggcagtaaccc |
| | | cgagccagatgccaccccgttccaggaaggtttgaggaccttcgaccagctggacgccatatcta |
| | | gtttgcccacacccagtgacatctttgtgtcctactctactttcccaggttttgtttcctggaggga |
| | | ccccaagagtggctcctggtacgttgagaccctggacgacatctttgagcagtgggctcactctgaag |
| | | acctgcagtccctcctgcttagggtcgctaatgctgtttcggtgaaagggatttataaacagatgcct |
| | | ggttgctttaatttcctccggaaaaaacttttctttaaaacatcatagttggatagttgtggaaaga |
| | | gtcaaatggctctcctcaagcgtattcaacaaggggctgaaggatgcccagaaggtaccccattgta |
| | | tgggatctgatctggggcctcggtgcacatgctttacatgtgtttagtcgaggttaaaaaacgtcta |
| | | ggccccccgaaccacggggacgtggttttcctttgaaaaacacgatgataatggccacagaattcat |
| | | ggtgagcaagggcgaggagctgatcaaggagaacatgcacagcaagctgtacctggaaggcagcgtga |
| | | acggccaccagttcaagtgcacccacgaaggggagggcaagccctacgagggcacccagaccaacagg |
| | | atcaaggtggtggagggaggccccctgccgttcgcattcgacatcctggccaccatgtttatgtacgg |
| | | gagcaaggccttcatcaagtaccccaagggcctccccgattattttaagcagtccttccctgagggct |
| | | tcacatgggagagagtcatggtgttcgaagacgggggcgtgctgaccgccacccaggacaccagcct |
| | | ccaggacggctgcctcatctacaacgtgaagctgagaggggtgaacttcccagccaacggccccgtg |
| | | atgaagcagacaacactgggctgggagcccagcaccgagaccctgtaccccgctgacggcgccctgg |
| | | aaggcagatgcgacatggccctgaagctcgtggggggggccacctgcactgcaacttcaagaccacat |
| | | acaaatccaagaaacccgctacaaacctcaagatgcccggcgtccactacgtggaccgcagactgga |
| | | aagaatcaaggaggccgacaacgagacctacgtcgagcagcacgaggtggctgtggccagatactgc |
| | | gacctccctagcaaactggggcacaaacttaatggCATGGACGAGCTGTACAAGtga |
| SB07352 | 186 | ATGgctggagacatgagagctgccaacctttggccaagcccgctcatgatcaaacgctctaagaagaa |
| ERT2*mut88- | | cagcctggccttgtccctgacggccgaccagatggtcagtgccttgttggatgctgagccccccatac |
| Casp9-IRES- | | tctattccgagtatgatcctaccagacccttcagtgaagcttcgatgatgggcttactgaccaacctg |
| RFP | | gcagacagggagCTGGTTCACATGATCAACTGGGCGAAGAGGGTGCCAGGCTTTGTGG |
| Nucleotide | | ATTTGACCCTCCATGATCAGGTCCACCTTCTAGAATGTGCCTGGCT |
| Sequence | | AGAGATCCTGATGATTGGTGTGGTCTGGCGCTCCATGGAGCACCC |
| IRES-RFP in | | AGTGAAGCTACTGTTTGCTCCTAACTTGCTCTTGGACAGGGACGAG |
| bold italic | | GGAAAATGTGTAGAGGGCATGGTGGAGATCTTCGACATGCTGCTG |
| | | GCTACATCATCTCGGTTCCGCATGATGAATCTGCAGGGAGAGGAG |
| | | TTTGTGTGCCTCAAATCTATTATTTTGCTTAATTCTGGAGTGTACAC |
| | | ATTTCTGCCCAGCACCCTGAAGTCTCTGGAAGAGAAGGACCATAT |
| | | CCACCGAGTCCTGGACAAGATCACAGACACTTTGATCCACCTGAT |
| | | GGCCAAGGCAGGCCTGACCCTGCAGCAGCAGCACCAGCGGCTGGC |
| | | CCAGCTCCTCCTCATCCTCTCCCACATCAGGCACATGAGTAACAAA |
| | | GGCATGGAGTTTctgtacagcatgaagtgcaagaacgtggtgcccctctatGaCctgctgctgga |
| | | ggcggcggacgcccaccgcctacatgcgcccactagccgtggaggggcatccgtggaggagacggac |
| | | caaagccacttggccactgcgggctctacttcatcgcattccttgcaaaagtattacatcacggggga |
| | | ggcagagggtttccctgccacagggggtggaggttcagggggtggaggttcaggtggtggcggtagt |
| | | gtcgatggcttcgatgtcggtgctcttgagagtttgaggggaaatgcagatttggcttacatcctgag |
| | | catggagccctgtggccactgcctcattatcaacaatgtgaacttctgccgtgagtccgggctccgca |
| | | cccgcactggctccaacatcgactgtgagaagttgcggcgtcgcttctcctcgctgcatttcatggt |
| | | ggaggtgaagggcgacctgactgccaagaaaatggtgctggctttgctggagctggcgcggcaggac |
| | | cacggtgctctggactgctgcgtggtggtcattctctctcacggctgtcaggccagccacctgcag |
| | | ttcccaggggctgtctacggcacagatggatgccctgtgtcggtcgagaagattgtgaacatcttcaa |
| | | tgggaccagctgccccagcctgggagggaagcccaagctctttttcatccaggcctgtggtggggag |
| | | cagaaagaccatgggtttgaggtggcctccacttcccctgaagacgagtcccctggcagtaaccccga |
| | | gccagatgccaccccgttccaggaaggtttgaggaccttcgaccagctggacgccatatctagtttgc |
| | | ccacacccagtgacatctttgtgtcctactctactttcccaggttttgtttcctggagggaccccaag |
| | | agtggctcctggtacgttgagaccctggacgacatctttgagcagtgggctcactctgaagacctgca |
| | | gtccctcctgcttagggtcgctaatgctgtttcggtgaaagggatttataaacagatgcctggttgct |
| | | ttaatttcctccggaaaaaacttttctttaaaacatcatagttggatagttgtggaaagagtcaaa |
| | | tggctctcctcaagcgtattcaacaaggggctgaaggatgcccagaaggtaccccattgtatgggat |
| | | ctgatctggggcctcggtgcacatgctttacatgtgtttagtcgaggttaaaaaacgtctaggcccc |
| | | ccgaaccacggggacgtggttttcctttgaaaaacacgatgataatggccacagaattcatggtgag |
| | | caagggcgaggagctgatcaaggagaacatgcacagcaagctgtacctggaaggcagcgtgaacggcc |
| | | accagttcaagtgcacccacgaaggggagggcaagccctacgagggcacccagaccaacaggatcaa |
| | | ggtggtggagggaggccccctgccgttcgcattcgacatcctggccaccatgtttatgtacgggagca |
| | | aggccttcatcaagtaccccaagggcctccccgattattttaagcagtccttccctgagggcttcac |
| | | atgggagagagtcatggtgttcgaagacgggggcgtgctgaccgccacccaggacaccagcctcca |
| | | ggacggctgcctcatctacaacgtgaagctgagaggggtgaacttcccagccaacggccccgtgatga |
| | | agcagacaacactgggctgggagcccagcaccgagaccctgtaccccgctgacggcgccctggaagg |
| | | cagatgcgacatggccctgaagctcgtggggggggccacctgcactgcaacttcaagaccacataca |
| | | aatccaagaaacccgctacaaacctcaagatgcccggcgtccactacgtggaccgcagactggaaa |
| | | gaatcaaggaggccgacaacgagacctacgtcgagcagcacgaggtggctgtggcc |
| | | agatactgcgacctccctagcaaactggggcacaaacttaatggCATGGACGAGCTGTACA |
| | | AGtga |
| SB07353 | 187 | ATGgctggagacatgagagctgccaacctttggccaagcccgctcatgatcaaacgctctaagaagaa |
| ERT2*mut63- | | cagcctggccttgtccctgacggccgaccagatggtcagtgccttgttggatgctgagccccccatac |
| Casp9-IRES- | | tctattccgagtatgatcctaccagacccttcagtgaagcttcgatgatgggcttactgaccaacctg |
| RFP | | gcagacagggagATCGTTCACATGATCAACTGGGCGAAGAGGGTGCCAGGCTTTGTGG |
| Nucleotide | | ATTTGACCCTCCATGATCAGGTCCACCTTCTAGAATGTGCCTGGCT |
| Sequence | | AGAGATCCTGATGATTGGTGTGGTCTGGCGCTCCATGGAGCACCC |
| IRES-RFP in | | AGTGAAGCTACTGTTTGCTCCTAACTTGCTCTTGGACAGGGACGAG |
| bold italic | | GGAAAATGTGTAGAGGGCCTGGTGGAGATCTTCGACATGCTGCTG |
| | | GCTACATCATCTCGGTTCCGCATGATGAATCTGCAGGGAGAGGAG |
| | | TTTGTGTGCCTCAAATCTATTATTTTGCTTAATTCTGGAGTGTACAC |
| | | ATTTCTGTCCAGCACCCTGAAGTCTCTGGAAGAGAAGGACCATATC |
| | | CACCGAGTCCTGGACAAGATCACAGACACTTTGATCCACCTGATG |
| | | GCCAAGGCAGGCCTGACCCTGCAGCAGCAGCACCAGCGGCTGGCC |
| | | CAGCTCCTCCTCATCCTCTCCCACATCAGGCACGCCAGTAACAAAG |
| | | GCATGGAGTTTctgtacagcatgaagtgcaagaacgtggtgcccctctatGaCctgctgctggagg |
| | | cggcggacgcccaccgcctacatgcgcccactagccgtggaggggcatccgtggaggagacggaccaa |
| | | agccacttggccactgcgggctctacttcatcgcattccttgcaaaagtattacatcacgggggagg |
| | | cagagggtttccctgccacagggggtggaggttcagggggtggaggttcaggtggtggcggtagtgtc |
| | | gatggcttcgatgtcggtgctcttgagagtttgaggggaaatgcagatttggcttacatcctgagca |
| | | tggagccctgtggccactgcctcattatcaacaatgtgaacttctgccgtgagtccgggctccgcac |
| | | ccgcactggctccaacatcgactgtgagaagttgcggcgtcgcttctcctcgctgcatttcatggtgg |
| | | aggtgaagggcgacctgactgccaagaaaatggtgctggctttgctggagctggcgcggcaggacca |
| | | cggtgctctggactgctgcgtggtggtcattctctctcacggctgtcaggccagccacctgcagttc |
| | | ccaggggctgtctacggcacagatggatgccctgtgtcggtcgagaagattgtgaacatcttcaatgg |
| | | gaccagctgccccagcctgggagggaagcccaagctctttttcatccaggcctgtggtggggagcaga |
| | | aagaccatgggtttgaggtggcctccacttcccctgaagacgagtcccctggcagtaaccccgagc |
| | | cagatgccaccccgttccaggaaggtttgaggaccttcgaccagctggacgccatatctagttt |
| | | gcccacacccagtgacatctttgtgtcctactctactttcccaggttttgtttcctggagggacccca |
| | | agagtggctcctggtacgttgagaccctggacgacatctttgagcagtgggctcactctgaagacct |
| | | gcagtccctcctgcttagggtcgctaatgctgtttcggtgaaagggatttataaacagatgcctggt |
| | | tgctttaatttcctccggaaaaaacttttctttaaaacatcatagttggatagttgtggaaagagtc |
| | | aaatggctctcctcaagcgtattcaacaaggggctgaaggatgcccagaaggtaccccattgtatg |
| | | ggatctgatctggggcctcggtgcacatgctttacatgtgtttagtcgaggttaaaaaacgtctaggc |
| | | cccccgaaccacggggacgtggttttcctttgaaaaacacgatgataatggccacagaattcatggtg |
| | | agcaagggcgaggagctgatcaaggagaacatgcacagcaagctgtacctggaaggcagcgtgaacgg |
| | | ccaccagttcaagtgcacccacgaaggggagggcaagccctacgagggcacccagaccaacaggatc |
| | | aaggtggtggagggaggccccctgccgttcgcattcgacatcctggccaccatgtttatgtacggga |
| | | gcaaggccttcatcaagtaccccaagggcctccccgattattttaagcagtc |
| | | cttccctgagggcttcacatgggagagagtcatggtgttcgaagacgggggcgtgctgaccgccaccc |
| | | aggacaccagcctccaggacggctgcctcatctacaacgtgaagctgagaggggtgaacttcccagcc |
| | | aacggccccgtgatgaagcagacaacactgggctgggagcccagcaccgagaccctgtaccccgctga |
| | | cggcgccctggaaggcagatgcgacatggccctgaagctcgtggggggggccacctgcactgcaactt |
| | | caagaccacatacaaatccaagaaacccgctacaaacctcaagatgcccggcgtccactacgtggacc |
| | | gcagactggaaagaatcaaggaggccgacaacgagacctacgtcgagcagcacgaggtggctgtggcc |
| | | agatactgcgacctccctagcaaactggggcacaaacttaatggCATGGACGAGCTGTACAAGt |
| | | ga |
| SB07354 | 188 | ATGgctggagacatgagagctgccaacctttggccaagcccgctcatgatcaaacgctctaagaagaa |
| ERT2*mut41- | | cagcctggccttgtccctgacggccgaccagatggtcagtgccttgttggatgctgagccccccatac |
| Casp9-IRES- | | tctattccgagtatgatcctaccagacccttcagtgaagcttcgatgatgggcttactgaccaacctg |
| RFP | | gcagacagggagATCGTTCACATGATCAACTGGGCGAAGAGGGTGCCAGGCTTTGTGG |
| Nucleotide | | ATTTGACCCTCCATGATCAGGTCCACCTTCTAGAATGTGCCTGGCT |
| Sequence | | AGAGATCCTGATGATTGGTGTGGTCTGGCGCTCCATGGAGCACCC |
| | | AGTGAAGCTACTGTTTGCTCCTAACTTGGTGTTGGACAGGGACGAG |
| | | GGAAAATGTGTAGAGGGCATGGTGGAGATCTTCGACATGCTGCTG |
| | | GCTACATCATCTCGGTTCCGCATGATGAATCTGCAGGGAGAGGAG |
| | | TTTGTGTGCCTCAAATCTATTATTTTGCTTAATTCTGGAGTGTACAC |
| IRES-RFP in | | ATTTCTGTCCAGCACCCTGAAGTCTCTGGAAGAGAAGGACCATATC |
| bold italic | | CACCGAGTCCTGGACAAGATCACAGACACTTTGATCCACCTGATG |
| | | GCCAAGGCAGGCCTGACCCTGCAGCAGCAGCACCAGCGGCTGGCC |
| | | CAGCTCCTCCTCATCCTCTCCCACATCAGGCACATGAGTAACAAAG |
| | | GCATGGAGCTGctgtacagcatgaagtgcaagaacgtggtgcccctctatGaCctgctgctggag |
| | | gcggcggacgcccaccgcctacatgcgcccactagccgtggaggggcatccgtggaggagacggacca |
| | | aagccacttggccactgcgggctctacttcatcgcattccttgcaaaagtattacatcacgggggag |
| | | gcagagggtttccctgccacagggggtggaggttcagggggtggaggttcaggtggtggcggtagtg |
| | | tcgatggcttcgatgtcggtgctcttgagagtttgaggggaaatgcagatttggcttacatcctgag |
| | | catggagccctgtggccactgcctcattatcaacaatgtgaacttctgccgtgagtccgggctccgc |
| | | acccgcactggctccaacatcgactgtgagaagttgcggcgtcgcttctcctcgctgcatttcatggt |
| | | ggaggtgaagggcgacctgactgccaagaaaatggtgctggctttgctggagctggcgcggcaggac |
| | | cacggtgctctggactgctgcgtggtggtcattctctctcacggctgtcaggccagccacctgcagt |
| | | tcccaggggctgtctacggcacagatggatgccctgtgtcggtcgagaagattgtgaacatcttcaat |
| | | gggaccagctgccccagcctgggagggaagcccaagctctttttcatccaggcctgtggtggggagca |
| | | gaaagaccatgggtttgaggtggcctccacttcccctgaagacgagtcccctggcagtaaccccgagc |
| | | cagatgccaccccgttccaggaaggtttgaggaccttcgaccagctggacgccatatctagtttgccc |
| | | acacccagtgacatctttgtgtcctactctactttcccaggttttgtttcctggagggaccccaagag |
| | | tggctcctggtacgttgagaccctggacgacatctttgagcagtgggctcactctgaagacctgcagt |
| | | ccctcctgcttagggtcgctaatgctgtttcggtgaaagggatttataaacagatgcctggttgctt |
| | | taatttcctccggaaaaaacttttctttaaaacatcatagttggatagttgtggaaagagtcaaatgg |
| | | ctctcctcaagcgtattcaacaaggggctgaaggatgcccagaaggtaccccattgtatgggatctga |
| | | tctggggcctcggtgcacatgctttacatgtgtttagtcgaggttaaaaaacgtctaggccccccga |
| | | accacggggacgtggttttcctttgaaaaacacgatgataatggccacagaattcatggtgagcaagg |
| | | gcgaggagctgatcaaggagaacatgcacagcaagctgtacctggaaggcagcgtgaacggccacca |
| | | gttcaagtgcacccacgaaggggagggcaagccctacgagggcacccagaccaacaggatcaaggtgg |
| | | tggagggaggccccctgccgttcgcattcgacatcctggccaccatgtttatgtacgggagcaagg |
| | | ccttcatcaagtaccccaagggcctccccgattattttaagcagtccttccctgagggcttcaca |
| | | tgggagagagtcatggtgttcgaagacgggggcgtgctgaccgccaccc |
| | | aggacaccagcctccaggacggctgcctcatctacaacgtgaagctgagaggggtgaacttcccagcc |
| | | aacggccccgtgatgaagcagacaacactgggctgggagcccagcaccgagaccctgtaccccgctga |
| | | cggcgccctggaaggcagatgcgacatggccctgaagctcgtggggggggccacctgcactgcaactt |
| | | caagaccacatacaaatccaagaaacccgctacaaacctcaagatgcccggcgtccactacgtggacc |
| | | gcagactggaaagaatcaaggaggccgacaacgagacctacgtcgagcagcacgaggtggctgtggcc |
| | | agatactgcgacctccctagcaaactggggcacaaacttaatggCATGGACGAGCTGTACA |
| | | AGtga |
| SB07351_1 | 189 | ATGgctggagacatgagagctgccaacctttggccaagcccgctcatgatcaaacgctc |
| ERT2*mut81- | | taagaagaacagcctggccttgtccctgacggccgaccagatggtcagtgccttgttggat |
| Casp9 | | gctgagccccccatactctattccgagtatgatcctaccagacccttcagtgaagcttcgatg |
| Nucleotide | | atgggcttactgaccaacctggcagacagggagCTGGTTCACATGATCAA |
| Sequence (not | | CTGGGCGAAGAGGGTGCCAGGCTTTGTGGATTTGACCC |
| including stop | | TCCATGATCAGGTCCACCTTCTAGAATGTGCCTGGATG |
| codon) | | GAGATCCTGATGATTGGTGTGGTCTGGCGCTCCATGGA |
| | | GCACCCAGTGAAGCTACTGTTTGCTCCTAACTTGCTCTT |
| | | GGACAGGGACCAGGGAAAATGTGTAGAGGGCCTGGTG |
| | | GAGATCTTCGACATGCTGCTGGCTACATCATCTCGGTT |
| | | CCGCATGATGAATCTGCAGGGAGAGGAGTTTGTGTGCC |
| | | TCAAATCTATTATTTTGCTTAATTCTGGAGTGTACACAT |
| | | TTCTGCCCAGCACCCTGAAGTCTCTGGAAGAGAAGGAC |
| | | CATATCCACCGAGTCCTGGACAAGATCACAGACACTTT |
| | | GATCCACCTGATGGCCAAGGCAGGCCTGACCCTGCAG |
| | | CAGCAGCACCAGCGGCTGGCCCAGCTCCTCCTCATCCT |
| | | CTCCCACATCAGGCACATGAGTAACAAAGGCATGGAG |
| | | CTGctgtacagcatgaagtgcaagaacgtggtgcccctctatGaCctgctgctggagg |
| | | cggcggacgcccaccgcctacatgcgcccactagccgtggaggggcatccgtggagga |
| | | gacggaccaaagccacttggccactgcgggctctacttcatcgcattccttgcaaaagtatt |
| | | acatcacgggggaggcagagggtttccctgccacagggggtggaggttcagggggtgg |
| | | aggttcaggtggtggcggtagtgtcgatggcttcgatgtcggtgctcttgagagtttgaggg |
| | | gaaatgcagatttggcttacatcctgagcatggagccctgtggccactgcctcattatcaaca |
| | | atgtgaacttctgccgtgagtccgggctccgcacccgcactggctccaacatcgactgtga |
| | | gaagttgcggcgtcgcttctcctcgctgcatttcatggtggaggtgaagggcgacctgactg |
| | | ccaagaaaatggtgctggctttgctggagctggcgcggcaggaccacggtgctctggact |
| | | gctgcgtggtggtcattctctctcacggctgtcaggccagccacctgcagttcccaggggct |
| | | gtctacggcacagatggatgccctgtgtcggtcgagaagattgtgaacatcttcaatgggac |
| | | cagctgccccagcctgggagggaagcccaagctctttttcatccaggcctgtggtgggga |
| | | gcagaaagaccatgggtttgaggtggcctccacttcccctgaagacgagtcccctggcagt |
| | | aaccccgagccagatgccaccccgttccaggaaggtttgaggaccttcgaccagctggac |
| | | gccatatctagtttgcccacacccagtgacatctttgtgtcctactctactttcccaggttttgttt |
| | | cctggagggaccccaagagtggctcctggtacgttgagaccctggacgacatctttgagc |
| | | agtgggctcactctgaagacctgcagtccctcctgcttagggtcgctaatgctgtttcggtga |
| | | aagggatttataaacagatgcctggttgctttaatttcctccggaaaaaacttttctttaaaacat |
| | | ca |
| SB07352_1 | 190 | ATGgctggagacatgagagctgccaacctttggccaagcccgctcatgatcaaacgctc |
| ERT2*mut88- | | taagaagaacagcctggccttgtccctgacggccgaccagatggtcagtgccttgttggat |
| Casp9 | | gctgagccccccatactctattccgagtatgatcctaccagacccttcagtgaagcttcgatg |
| Nucleotide | | atgggcttactgaccaacctggcagacagggagCTGGTTCACATGATCAA |
| Sequence (not | | CTGGGCGAAGAGGGTGCCAGGCTTTGTGGATTTGACCC |
| including stop | | TCCATGATCAGGTCCACCTTCTAGAATGTGCCTGGCTA |
| codon) | | GAGATCCTGATGATTGGTGTGGTCTGGCGCTCCATGGA |
| | | GCACCCAGTGAAGCTACTGTTTGCTCCTAACTTGCTCTT |
| | | GGACAGGGACGAGGGAAAATGTGTAGAGGGCATGGTG |
| | | GAGATCTTCGACATGCTGCTGGCTACATCATCTCGGTT |
| | | CCGCATGATGAATCTGCAGGGAGAGGAGTTTGTGTGCC |
| | | TCAAATCTATTATTTTGCTTAATTCTGGAGTGTACACAT |
| | | TTCTGCCCAGCACCCTGAAGTCTCTGGAAGAGAAGGAC |
| | | CATATCCACCGAGTCCTGGACAAGATCACAGACACTTT |
| | | GATCCACCTGATGGCCAAGGCAGGCCTGACCCTGCAG |
| | | CAGCAGCACCAGCGGCTGGCCCAGCTCCTCCTCATCCT |
| | | CTCCCACATCAGGCACATGAGTAACAAAGGCATGGAG |
| | | TTTctgtacagcatgaagtgcaagaacgtggtgcccctctatGaCctgctgctggaggc |
| | | ggcggacgcccaccgcctacatgcgcccactagccgtggaggggcatccgtggaggag |
| | | acggaccaaagccacttggccactgcgggctctacttcatcgcattccttgcaaaagtatta |
| | | catcacgggggaggcagagggtttccctgccacagggggtggaggttcagggggtgga |
| | | ggttcaggtggtggcggtagtgtcgatggcttcgatgtcggtgctcttgagagtttgagggg |
| | | aaatgcagatttggcttacatcctgagcatggagccctgtggccactgcctcattatcaacaa |
| | | tgtgaacttctgccgtgagtccgggctccgcacccgcactggctccaacatcgactgtgag |
| | | aagttgcggcgtcgcttctcctcgctgcatttcatggtggaggtgaagggcgacctgactgc |
| | | caagaaaatggtgctggctttgctggagctggcgcggcaggaccacggtgctctggactg |
| | | ctgcgtggtggtcattctctctcacggctgtcaggccagccacctgcagttcccaggggctg |
| | | tctacggcacagatggatgccctgtgtcggtcgagaagattgtgaacatcttcaatgggacc |
| | | agctgccccagcctgggagggaagcccaagctctttttcatccaggcctgtggtggggag |
| | | cagaaagaccatgggtttgaggtggcctccacttcccctgaagacgagtcccctggcagta |
| | | accccgagccagatgccaccccgttccaggaaggtttgaggaccttcgaccagctggacg |
| | | ccatatctagtttgcccacacccagtgacatctttgtgtcctactctactttcccaggttttgtttc |
| | | ctggagggaccccaagagtggctcctggtacgttgagaccctggacgacatctttgagca |
| | | gtgggctcactctgaagacctgcagtccctcctgcttagggtcgctaatgctgtttcggtgaa |
| | | agggatttataaacagatgcctggttgctttaatttcctccggaaaaaacttttctttaaaacatc |
| | | a |
| SB07353_1 | 191 | ATGgctggagacatgagagctgccaacctttggccaagcccgctcatgatcaaacgctc |
| ERT2*mut63- | | taagaagaacagcctggccttgtccctgacggccgaccagatggtcagtgccttgttggat |
| Casp9 | | gctgagccccccatactctattccgagtatgatcctaccagacccttcagtgaagcttcgatg |
| Nucleotide | | atgggcttactgaccaacctggcagacagggagATCGTTCACATGATCAA |
| Sequence (not | | CTGGGCGAAGAGGGTGCCAGGCTTTGTGGATTTGACCC |
| including stop | | TCCATGATCAGGTCCACCTTCTAGAATGTGCCTGGCTA |
| codon) | | GAGATCCTGATGATTGGTGTGGTCTGGCGCTCCATGGA |
| | | GCACCCAGTGAAGCTACTGTTTGCTCCTAACTTGCTCTT |
| | | GGACAGGGACGAGGGAAAATGTGTAGAGGGCCTGGTG |
| | | GAGATCTTCGACATGCTGCTGGCTACATCATCTCGGTT |
| | | CCGCATGATGAATCTGCAGGGAGAGGAGTTTGTGTGCC |
| | | TCAAATCTATTATTTTGCTTAATTCTGGAGTGTACACAT |
| | | TTCTGTCCAGCACCCTGAAGTCTCTGGAAGAGAAGGAC |
| | | CATATCCACCGAGTCCTGGACAAGATCACAGACACTTT |
| | | GATCCACCTGATGGCCAAGGCAGGCCTGACCCTGCAG |
| | | CAGCAGCACCAGCGGCTGGCCCAGCTCCTCCTCATCCT |
| | | CTCCCACATCAGGCACGCCAGTAACAAAGGCATGGAG |
| | | TTTctgtacagcatgaagtgcaagaacgtggtgcccctctatGaCctgctgctggaggc |
| | | ggcggacgcccaccgcctacatgcgcccactagccgtggaggggcatccgtggaggag |
| | | acggaccaaagccacttggccactgcgggctctacttcatcgcattccttgcaaaagtatta |
| | | catcacgggggaggcagagggtttccctgccacagggggtggaggttcagggggtgga |
| | | ggttcaggtggtggcggtagtgtcgatggcttcgatgtcggtgctcttgagagtttgagggg |
| | | aaatgcagatttggcttacatcctgagcatggagccctgtggccactgcctcattatcaacaa |
| | | tgtgaacttctgccgtgagtccgggctccgcacccgcactggctccaacatcgactgtgag |
| | | aagttgcggcgtcgcttctcctcgctgcatttcatggtggaggtgaagggcgacctgactgc |
| | | caagaaaatggtgctggctttgctggagctggcgcggcaggaccacggtgctctggactg |
| | | ctgcgtggtggtcattctctctcacggctgtcaggccagccacctgcagttcccaggggctg |
| | | tctacggcacagatggatgccctgtgtcggtcgagaagattgtgaacatcttcaatgggacc |
| | | agctgccccagcctgggagggaagcccaagctctttttcatccaggcctgtggtggggag |
| | | cagaaagaccatgggtttgaggtggcctccacttcccctgaagacgagtcccctggcagta |
| | | accccgagccagatgccaccccgttccaggaaggtttgaggaccttcgaccagctggacg |
| | | ccatatctagtttgcccacacccagtgacatctttgtgtcctactctactttcccaggttttgtttc |
| | | ctggagggaccccaagagtggctcctggtacgttgagaccctggacgacatctttgagca |
| | | gtgggctcactctgaagacctgcagtccctcctgcttagggtcgctaatgctgtttcggtgaa |
| | | agggatttataaacagatgcctggttgctttaatttcctccggaaaaaacttttctttaaaacatc |
| | | a |
| SB07354_1 | 192 | ATGgctggagacatgagagctgccaacctttggccaagcccgctcatgatcaaacgctc |
| ERT2*mut41- | | taagaagaacagcctggccttgtccctgacggccgaccagatggtcagtgccttgttggat |
| Casp9 | | gctgagccccccatactctattccgagtatgatcctaccagacccttcagtgaagcttcgatg |
| Nucleotide | | atgggcttactgaccaacctggcagacagggagATCGTTCACATGATCAA |
| Sequence (not | | CTGGGCGAAGAGGGTGCCAGGCTTTGTGGATTTGACCC |
| including stop | | TCCATGATCAGGTCCACCTTCTAGAATGTGCCTGGCTA |
| codon) | | GAGATCCTGATGATTGGTGTGGTCTGGCGCTCCATGGA |
| | | GCACCCAGTGAAGCTACTGTTTGCTCCTAACTTGGTGT |
| | | TGGACAGGGACGAGGGAAAATGTGTAGAGGGCATGGT |
| | | GGAGATCTTCGACATGCTGCTGGCTACATCATCTCGGT |
| | | TCCGCATGATGAATCTGCAGGGAGAGGAGTTTGTGTGC |
| | | CTCAAATCTATTATTTTGCTTAATTCTGGAGTGTACACA |
| | | TTTCTGTCCAGCACCCTGAAGTCTCTGGAAGAGAAGGA |
| | | CCATATCCACCGAGTCCTGGACAAGATCACAGACACTT |
| | | TGATCCACCTGATGGCCAAGGCAGGCCTGACCCTGCAG |
| | | CAGCAGCACCAGCGGCTGGCCCAGCTCCTCCTCATCCT |
| | | CTCCCACATCAGGCACATGAGTAACAAAGGCATGGAG |
| | | CTGctgtacagcatgaagtgcaagaacgtggtgcccctctatGaCctgctgctggagg |
| | | cggcggacgcccaccgcctacatgcgcccactagccgtggaggggcatccgtggagga |
| | | gacggaccaaagccacttggccactgcgggctctacttcatcgcattccttgcaaaagtatt |
| | | acatcacgggggaggcagagggtttccctgccacagggggtggaggttcagggggtgg |
| | | aggttcaggtggtggcggtagtgtcgatggcttcgatgtcggtgctcttgagagtttgaggg |
| | | gaaatgcagatttggcttacatcctgagcatggagccctgtggccactgcctcattatcaaca |
| | | atgtgaacttctgccgtgagtccgggctccgcacccgcactggctccaacatcgactgtga |
| | | gaagttgcggcgtcgcttctcctcgctgcatttcatggtggaggtgaagggcgacctgactg |
| | | ccaagaaaatggtgctggctttgctggagctggcgcggcaggaccacggtgctctggact |
| | | gctgcgtggtggtcattctctctcacggctgtcaggccagccacctgcagttcccaggggct |
| | | gtctacggcacagatggatgccctgtgtcggtcgagaagattgtgaacatcttcaatgggac |
| | | cagctgccccagcctgggagggaagcccaagctctttttcatccaggcctgtggtgggga |
| | | gcagaaagaccatgggtttgaggtggcctccacttcccctgaagacgagtcccctggcagt |
| | | aaccccgagccagatgccaccccgttccaggaaggtttgaggaccttcgaccagctggac |
| | | gccatatctagtttgcccacacccagtgacatctttgtgtcctactctactttcccaggttttgttt |
| | | cctggagggaccccaagagtggctcctggtacgttgagaccctggacgacatctttgagc |
| | | agtgggctcactctgaagacctgcagtccctcctgcttagggtcgctaatgctgtttcggtga |
| | | aagggatttataaacagatgcctggttgctttaatttcctccggaaaaaacttttctttaaaacat |
| | | ca |
| ERT2*mut81- | 193 | AGDMRAANLWPSPLMIKRSKKNSLALSLTADQMVSALLDAEPPIL |
| Casp9 | | YSEYDPTRPFSEASMMGLLTNLADRELVHMINWAKRVPGFVDLT |
| (Amino acid | | LHDQVHLLECAWMEILMIGVVWRSMEHPVKLLFAPNLLLDRDQ |
| sequence) | | GKCVEGLVEIFDMLLATSSRFRMMNLQGEEFVCLKSIILLNSGVY |
| | | TFLPSTLKSLEEKDHIHRVLDKITDTLIHLMAKAGLTLQQQHQRL |
| | | AQLLLILSHIRHMSNKGMELLYSMKCKNVVPLYDLLLEAADAHR |
| | | LHAPTSRGGASVEETDQSHLATAGSTSSHSLQKYYITGEAEGFPAT |
| | | GGGGSGGGGSGGGGSVDGFMDVGALESLRGNADLAYILSMEPCGHC |
| | | LIINNVNFCRESGLRTRTGSNIDCEKLRRRFSSLHFMVEVKGDLTAKK |
| | | MVLALLELARQDHGALDCCVVVILSHGCQASHLQFPGAVYGTDGCP |
| | | VSVEKIVNIFNGTSCPSLGGKPKLFFIQACGGEQKDHGFEVASTSPEDE |
| | | SPGSNPEPDATPFQEGLRTFDQLDAISSLPTPSDIFVSYSTFPGFVSWRD |
| | | PKSGSWYVETLDDIFEQWAHSEDLQSLLLRVANAVSVKGIYKQMPGC |
| | | FNFLRKKLFFKTS |
| | | Bold = ERT2*mut81 |
| | | Italics = linker |
| | | Plain = Casp9 |
| ERT2*mut88- | 194 | AGDMRAANLWPSPLMIKRSKKNSLALSLTADQMVSALLDAEPPIL |
| Casp9 | | YSEYDPTRPFSEASMMGLLTNLADRELVHMINWAKRVPGFVDLT |
| | | LHDQVHLLECAWLEILMIGVVWRSMEHPVKLLFAPNLLLDRDEG |
| | | KCVEGMVEIFDMLLATSSRFRMMNLQGEEFVCLKSIILLNSGVYT |
| | | FLPSTLKSLEEKDHIHRVLDKITDTLIHLMAKAGLTLQQQHQRLA |
| | | QLLLILSHIRHMSNKGMEFLYSMKCKNVVPLYDLLLEAADAHRL |
| | | HAPTSRGGASVEETDQSHLATAGSTSSHSLQKYYITGEAEGFPAT |
| | | GGGGSGGGGSGGGGSVDGFMDVGALESLRGNADLAYILSMEPCGHC |
| | | LIINNVNFCRESGLRTRTGSNIDCEKLRRRFSSLHFMVEVKGDLTAKK |
| | | MVLALLELARQDHGALDCCVVVILSHGCQASHLQFPGAVYGTDGCP |
| | | VSVEKIVNIFNGTSCPSLGGKPKLFFIQACGGEQKDHGFEVASTSPEDE |
| | | SPGSNPEPDATPFQEGLRTFDQLDAISSLPTPSDIFVSYSTFPGFVSWRD |
| | | PKSGSWYVETLDDIFEQWAHSEDLQSLLLRVANAVSVKGIYKQMPGC |
| | | FNFLRKKLFFKTS |
| | | Bold = ERT2*mut88 |
| | | Italics = linker |
| | | Plain = Casp9 |
| ERT2*mut63- | 195 | AGDMRAANLWPSPLMIKRSKKNSLALSLTADQMVSALLDAEPPIL |
| Casp9 | | YSEYDPTRPFSEASMMGLLTNLADREIVHMINWAKRVPGFVDLT |
| | | LHDQVHLLECAWLEILMIGVVWRSMEHPVKLLFAPNLLLDRDEG |
| | | KCVEGLVEIFDMLLATSSRFRMMNLQGEEFVCLKSIILLNSGVYT |
| | | FLSSTLKSLEEKDHIHRVLDKITDTLIHLMAKAGLTLQQQHQRLA |
| | | QLLLILSHIRHASNKGMEFLYSMKCKNVVPLYDLLLEAADAHRL |
| | | HAPTSRGGASVEETDQSHLATAGSTSSHSLQKYYITGEAEGFPAT |
| | | GGGGSGGGGSGGGGSVDGFMDVGALESLRGNADLAYILSMEPCGHC |
| | | LIINNVNFCRESGLRTRTGSNIDCEKLRRRFSSLHFMVEVKGDLTAKK |
| | | MVLALLELARQDHGALDCCVVVILSHGCQASHLQFPGAVYGTDGCP |
| | | VSVEKIVNIFNGTSCPSLGGKPKLFFIQACGGEQKDHGFEVASTSPEDE |
| | | SPGSNPEPDATPFQEGLRTFDQLDAISSLPTPSDIFVSYSTFPGFVSWRD |
| | | PKSGSWYVETLDDIFEQWAHSEDLQSLLLRVANAVSVKGIYKQMPGC |
| | | FNFLRKKLFFKTS |
| | | Bold = ERT2*mut63 |
| | | Italics = linker |
| | | Plain = Casp9 |
| ERT2*mut41- | 196 | AGDMRAANLWPSPLMIKRSKKNSLALSLTADQMVSALLDAEPPIL |
| Casp9 | | YSEYDPTRPFSEASMMGLLTNLADREIVHMINWAKRVPGFVDLT |
| | | LHDQVHLLECAWLEILMIGVVWRSMEHPVKLLFAPNLVLDRDEG |
| | | KCVEGMVEIFDMLLATSSRFRMMNLQGEEFVCLKSIILLNSGVYT |
| | | FLSSTLKSLEEKDHIHRVLDKITDTLIHLMAKAGLTLQQQHQRLA |
| | | QLLLILSHIRHMSNKGMELLYSMKCKNVVPLYDLLLEAADAHRL |
| | | HAPTSRGGASVEETDQSHLATAGSTSSHSLQKYYITGEAEGFPAT |
| | | GGGGSGGGGSGGGGSVDGFMDVGALESLRGNADLAYILSMEPCGHC |
| | | LIINNVNFCRESGLRTRTGSNIDCEKLRRRFSSLHFMVEVKGDLTAKK |
| | | MVLALLELARQDHGALDCCVVVILSHGCQASHLQFPGAVYGTDGCP |
| | | VSVEKIVNIFNGTSCPSLGGKPKLFFIQACGGEQKDHGFEVASTSPEDE |
| | | SPGSNPEPDATPFQEGLRTFDQLDAISSLPTPSDIFVSYSTFPGFVSWRD |
| | | PKSGSWYVETLDDIFEQWAHSEDLQSLLLRVANAVSVKGIYKQMPGC |
| | | FNFLRKKLFFKTS |
| | | Bold = ERT2*mut41 |
| | | Underlined = linker |
| | | Plain = Casp9 |

While the present disclosure has been particularly shown and described with reference to a preferred embodiment and various alternate embodiments, it will be understood by persons skilled in the relevant art that various changes in form and details can be made therein without departing from the spirit and scope of the present disclosure and appended claims.

All references, issued patents and patent applications cited within the body of the instant specification are hereby incorporated by reference in their entirety, for all purposes.

What is claimed is:

1. An inducible cell-death system comprising a polypeptide, wherein the polypeptide comprises a ligand binding domain and a cell death inducing domain, wherein the polypeptide is configured upon contact with a ligand of the ligand binding domain to generate a cell-death inducing signal in a cell in which the polypeptide is expressed, and wherein the ligand binding domain comprises a modified estrogen receptor ligand binding domain (ER-LBD) corresponding to a hormone binding domain of a reference human estrogen receptor sequence (SEQ ID NO: 1),
wherein the modified ER-LBD comprises
(a) a G400V amino acid substitution, an M543A amino acid substitution, an L544A amino acid substitution, and optionally a V595A amino acid substitution, with reference to SEQ ID NO: 1; and
(b) one or more additional amino acid substitutions, wherein the one or more additional amino acid substitutions are with reference to one or more regions selected from: positions 343-354, positions 380-392, positions 404-463, positions 517-540, and position 547 of SEQ ID NO: 1,
optionally wherein the modified ER-LBD has greater sensitivity to a non-endogenous ligand as compared to an ER-LBD comprising the amino acid sequence of SEQ ID NO: 2, optionally wherein the modified ER-LBD has greater sensitivity to a non-endogenous ligand as compared to an endogenous ligand as a result of the one or more additional amino acid substitutions, optionally wherein the modified ER-LBD has greater selectivity to a non-endogenous ligand as compared to an ER-LBD comprising the amino acid sequence of SEQ ID NO: 2,
optionally wherein the ligand binding domain of the first polypeptide monomer and the second polypeptide monomer comprise the same additional amino acid substitutions.

2. The inducible cell death system of claim 1, wherein
a. the one or more additional amino acid substitutions of the polypeptide, the first polypeptide monomer, and/or the second polypeptide monomer comprise an L391 substitution, optionally wherein the L391 substitution is L391V;
b. the one or more additional amino acid substitutions of the polypeptide, the first polypeptide monomer, and/or the second polypeptide monomer comprise an N413D mutation; or
C. the one or more additional amino acid substitutions of the polypeptide, the first polypeptide monomer, and/or the second polypeptide monomer comprise an L391V substitution and an N413D mutation;
d. the one or more additional amino acid substitutions of the polypeptide, the first polypeptide monomer, and/or the second polypeptide monomer comprises an H524 substitution, optionally wherein the H524 substitution is an H524L substitution or an H524F substitution;
e. the one or more additional amino acid substitutions of the polypeptide, the first polypeptide monomer, and/or the second polypeptide monomer comprises an M421L substitution;
f. the one or more additional amino acid substitutions of the polypeptide, the first polypeptide monomer, and/or the second polypeptide monomer comprises an S463P substitution;
g. the one or more additional amino acid substitutions of the polypeptide, the first polypeptide monomer, and/or the second polypeptide monomer comprises an M421L substitution and an S463P substitution;
h. the one or more additional amino acid substitutions of the polypeptide, the first polypeptide monomer, and/or the second polypeptide monomer comprises an L384M substitution;
i. the one or more additional amino acid substitutions of the polypeptide, the first polypeptide monomer, and/or the second polypeptide monomer comprises a L354I substitution;
j. the one or more additional amino acid substitutions of the polypeptide, the first polypeptide monomer, and/or the second polypeptide monomer comprises a Q414E substitution;
k. the one or more additional amino acid substitutions of the polypeptide, the first polypeptide monomer, and/or the second polypeptide monomer comprises a L354I substitution and a Q414E substitution;
l. the one or more additional amino acid substitutions of the polypeptide, the first polypeptide monomer, and/or the second polypeptide monomer comprises an L391V substitution, an N413D mutation, and an H524 substitution, optionally wherein the H524 substitution is an H524L substitution or an H524F substitution;
m. the one or more additional amino acid substitutions of the polypeptide, the first polypeptide monomer, and/or the second polypeptide monomer comprises an L391V substitution, an N413D mutation, an H524 substitution, and an M421L substitution, optionally wherein the H524 substitution is an H524L substitution or an H524F substitution;
n. the one or more additional amino acid substitutions of the polypeptide, the first polypeptide monomer, and/or the second polypeptide monomer comprises an L391V substitution, an N413D mutation, an H524 substitution, and an S463P substitution, optionally wherein the H524 substitution is an H524L substitution or an H524F substitution;
o. wherein the one or more additional amino acid substitutions of the polypeptide, the first polypeptide monomer, and/or the second polypeptide monomer comprises an L391V substitution, an N413D mutation, an H524 substitution, and an Q414E substitution, optionally wherein the H524 substitution is an H524L substitution or an H524F substitution;
p. the one or more additional amino acid substitutions of the polypeptide, the first polypeptide monomer, and/or the second polypeptide monomer comprises an L391V substitution, an N413D mutation, an H524 substitution, and an L354I substitution, optionally wherein the H524 substitution is an H524L substitution or an H524F substitution.

3. The inducible cell death system of claim 1, wherein the one or more additional amino acid substitutions of the polypeptide, the first polypeptide monomer, and/or the second polypeptide monomer are at one or more positions of SEQ ID NO: 1 selected from: 343, 344, 345, 346, 347, 348, 349, 350, 351, 352, 354, 380, 384, 386, 387, 388, 389, 391, 392, 404, 407, 409, 413, 414, 417, 418, 420, 421, 422, 424, 428, 463, 517, 521, 522, 524, 525, 526, 527, 528, 533, 534, 536, 537, 538, 539, 540, and 547,
optionally wherein:
i. the one or more positions comprise position 343 of SEQ ID NO: 1, optionally wherein the amino acid substitution at position 343 of SEQ ID NO: 1 is selected from the group consisting of: M343F, M343I, M343L, and M343V;
ii. the one or more positions comprise position 344 of SEQ ID NO: 1, optionally wherein the amino acid substitution at position 344 of SEQ ID NO: 1 is G344M;
iii. the one or more positions comprise position 345 of SEQ ID NO: 1, optionally wherein the amino acid substitution at position 345 of SEQ ID NO: 1 is L345S;
iv. the one or more positions comprise position 346 of SEQ ID NO: 1, optionally wherein the amino acid substitution at position 346 of SEQ ID NO: 1 is selected from the group consisting of: L346I, L346M, L346F, and L346V;
v. the one or more positions comprise position 347 of SEQ ID NO: 1, optionally wherein the amino acid substitution at position 347 of SEQ ID NO: 1 is selected from the group consisting of: T347D, T347E, T347F, T347I, T347K, T347L, T347M, T347N, T347Q, T347R, T347S, and T347V;
vi. the one or more positions comprise position 348 of SEQ ID NO: 1, optionally wherein the amino acid substitution at position 348 of SEQ ID NO: 1 is N348K;
vii. the one or more positions comprise position 349 of SEQ ID NO: 1, optionally wherein the amino acid substitution at position 349 of SEQ ID NO: 1 is selected from the group consisting of: L349I, L349M, L349F, and L349V;
viii. the one or more positions comprise position 350 of SEQ ID NO: 1, optionally wherein the amino acid substitution at position 350 of SEQ ID NO: 1 is selected from the group consisting of: A350F, A350I, A350L, A350M and A350V;
ix. the one or more positions comprise position 351 of SEQ ID NO: 1, optionally wherein the amino acid substitution at position 351 of SEQ ID NO: 1 is selected from the group consisting of: D351E, D351F, D351I, D351L, D351M, D351N, D351Q, and D351V;
x. the one or more positions comprise position 352 of SEQ ID NO: 1, optionally wherein the amino acid substitution at position 352 of SEQ ID NO: 1 is R352K;
xi. the one or more positions comprise position 354 of SEQ ID NO: 1, optionally wherein the amino acid substitution at position 354 of SEQ ID NO: 1 is selected from the group consisting of: L354I, L354M, L354F, and L354V;
xii. the one or more positions comprise position 380 of SEQ ID NO: 1, optionally wherein the amino acid substitution at position 380 of SEQ ID NO: 1 is E380Q;
xiii. the one or more positions comprise position 384 of SEQ ID NO: 1, optionally wherein the amino acid substitution at position 384 of SEQ ID NO: 1 is selected from the group consisting of: L384I, L384M, L384F, and L384V;
xiv. the one or more positions comprise position 386 of SEQ ID NO: 1, optionally wherein the amino acid substitution at position 386 of SEQ ID NO: 1 is I386V;
xv. the one or more positions comprise position 387 of SEQ ID NO: 1, optionally wherein the amino acid substitution at position 387 of SEQ ID NO: 1 is selected from the group consisting of: L387I, L387M, L387F, and L387V;
xvi. the one or more positions comprise position 388 of SEQ ID NO: 1, optionally wherein the amino acid substitution at position 388 of SEQ ID NO: 1 is selected from the group consisting of: M388I, M388L, and M388F;
xvii. the one or more positions comprise position 389 of SEQ ID NO: 1, optionally wherein the amino acid substitution at position 389 of SEQ ID NO: 1 is I389M;
xviii. the one or more positions comprise position 391 of SEQ ID NO: 1, optionally wherein the amino acid substitution at position 391 of SEQ ID NO: 1 is selected from the group consisting of: L391I, L391M, L391F, and L391V, optionally wherein the amino acid substitution at position 391 of SEQ ID NO: 1 is L391V;
xix. the one or more positions comprise position 392 of SEQ ID NO: 1, optionally wherein the amino acid substitution at position 392 of SEQ ID NO: 1 is V392M;
xx. the one or more positions comprise position 404 of SEQ ID NO: 1, optionally wherein the amino acid substitution at position 404 of SEQ ID NO: 1 is selected from the group consisting of: F404I, F404L, F404M, and F404V;
xxi. the one or more positions comprise position 407 of SEQ ID NO: 1, optionally wherein the amino acid substitution at position 407 of SEQ ID NO: 1 is N407D;
xxii. the one or more positions comprise position 409 of SEQ ID NO: 1, optionally wherein the amino acid substitution at position 409 of SEQ ID NO: 1 is L409V;
xxiii. the one or more positions comprise position 413 of SEQ ID NO: 1, optionally wherein the amino acid substitution at position 413 of SEQ ID NO: 1 is N413D;
xxiv. the one or more positions comprise position 414 of SEQ ID NO: 1, optionally wherein the amino acid substitution at position 414 of SEQ ID NO: 1 is Q414E;
xxv. the one or more positions comprise position 417 of SEQ ID NO: 1, optionally wherein the amino acid substitution at position 417 of SEQ ID NO: 1 is C417S;
xxvi. the one or more positions comprise position 418 of SEQ ID NO: 1, optionally the amino acid substitution at position 418 of SEQ ID NO: 1 is selected from the group consisting of: V418I, V418L, V418M, and V418F;
xxvii. the one or more positions comprise position 420 of SEQ ID NO: 1, optionally wherein the amino acid substitution at position 420 of SEQ ID NO: 1 is selected from the group consisting of: G420I, G420M, G420F, and G420V;
xxviii. the one or more positions comprise position 421 of SEQ ID NO: 1, optionally wherein the amino acid substitution at position 421 of SEQ ID NO: 1 is selected from the group consisting of: M421I, M421L, M421F, and M421V;
xxix. the one or more positions comprise position 422 of SEQ ID NO: 1, optionally wherein the amino acid substitution at position 422 of SEQ ID NO: 1 is V422I;
xxx. the one or more positions comprise position 424 of SEQ ID NO: 1, optionally wherein the amino acid substitution at position 424 of SEQ ID NO: 1 is selected from the group consisting of: 1424L, 1424M, 1424F, and I424V;
xxxi. the one or more positions comprise position 428 of SEQ ID NO: 1, optionally wherein the amino acid substitution at position 428 of SEQ ID NO: 1 is selected from the group consisting of: L428I, L428M, L428F, and L428V;
xxxii. the one or more positions comprise position 463 of SEQ ID NO: 1, optionally wherein the amino acid substitution at position 463 of SEQ ID NO: 1 is S463P;
xxxiii. the one or more positions comprise position 517 of SEQ ID NO: 1, optionally wherein the amino acid substitution at position 517 of SEQ ID NO: 1 is M517A;
xxxiv. the one or more positions comprise position 521 of SEQ ID NO: 1, optionally wherein the amino acid substitution at position 521 of SEQ ID NO: 1 is selected from the group consisting of: G521A, G521F, G521I, G521L, G521M, and G521V;
xxxv. the one or more positions comprise position 522 of SEQ ID NO: 1, optionally wherein the amino acid substitution at position 522 of SEQ ID NO: 1 is selected from the group consisting of: M522I, M522L, and M522V;
xxxvi. the one or more positions comprise position 524 of SEQ ID NO: 1, optionally wherein the amino acid substitution at position 524 of SEQ ID NO: 1 is selected from the group consisting of: H524A, H524I, H524L, H524F, and H524V;
xxxvii. the one or more positions comprise position 525 of SEQ ID NO: 1, optionally wherein the amino acid substitution at position 525 of SEQ ID NO: 1 is selected from the group consisting of: L525F, L525I, L525M, L525N, L525Q, L525S, L525T, and L525V;
xxxviii. the one or more positions comprise position 526 of SEQ ID NO: 1, optionally wherein the amino acid substitution at position 526 of SEQ ID NO: 1 is Y526L;
xxxix. the one or more positions comprise position 527 of SEQ ID NO: 1, optionally wherein the amino acid substitution at position 527 of SEQ ID NO: 1 is S527N;
xl. the one or more positions comprise position 528 of SEQ ID NO: 1, optionally wherein the amino acid substitution at position 528 of SEQ ID NO: 1 is selected from the group consisting of: M528F, M528I, and M528V;
xli. the one or more positions comprise position 533 of SEQ ID NO: 1, optionally wherein the amino acid substitution at position 533 of SEQ ID NO: 1 is selected from the group consisting of: V533F and V533W;
xlii. the one or more positions comprise position 534 of SEQ ID NO: 1, optionally wherein the amino acid substitution at position 534 of SEQ ID NO: 1 is selected from the group consisting of: V534Q and V534R;
xliii. the one or more positions comprise position 536 of SEQ ID NO: 1, optionally wherein the amino acid substitution at position 536 of SEQ ID NO: 1 is selected from the group consisting of: L536F, and L536M, L536R, and L536Y;
xliv. the one or more positions comprise position 537 of SEQ ID NO: 1, optionally wherein the amino acid substitution at position 537 of SEQ ID NO: 1 is selected from the group consisting of: Y537E and Y537S;
xlv. the one or more positions comprise position 538 of SEQ ID NO: 1, optionally wherein the amino acid substitution at position 538 of SEQ ID NO: 1 is selected from the group consisting of: D538G and D538K;
xlvi. the one or more positions comprise position 539 of SEQ ID NO: 1, optionally wherein the amino acid substitution at position 539 of SEQ ID NO: 1 is selected from the group consisting of: L539A and L539R;
xlvii. the one or more positions comprise position 540 of SEQ ID NO: 1, optionally wherein the amino acid substitution at position 540 of SEQ ID NO: 1 is selected from the group consisting of: L540A and L540F; and/or
xlviii. the one or more positions comprise position 547 of SEQ ID NO: 1, optionally wherein the amino acid substitution at position 547 of SEQ ID NO: 1 is H547A.

4. The inducible cell death system of claim 1, wherein:
a. the one or more additional amino acid substitutions of the polypeptide, the first polypeptide monomer, and/or the second polypeptide monomer are two amino acid substitutions, optionally wherein each of the two amino acid substitutions are at a position of SEQ ID NO: 1 selected from: 343, 345, 347, 348, 351, 354, 384, 387, 388, 389, 391, 392, 404, 418, 421, 521, 524, and 525, optionally wherein:
i. the two amino acid substitutions are at positions 345 and 348 of SEQ ID NO: 1, optionally wherein the amino acid substitution at position 345 of SEQ ID NO: 1 is L345S and the amino acid substitution at position 348 of SEQ ID NO: 1 is N348K;
ii. the two amino acid substitutions are at positions 384 and 389 of SEQ ID NO: 1, optionally wherein the amino acid substitution at position 384 of SEQ ID NO: 1 is L384M and the amino acid substitution at position 389 of SEQ ID NO: 1 is I389M;
iii. the two amino acid substitutions are at positions 421 and 392 of SEQ ID NO: 1, optionally wherein the amino acid substitution at position 421 of SEQ ID NO: 1 is M421I and the amino acid substitution at position 392 of SEQ ID NO: 1 is V392M;
iv. the two amino acid substitutions are at positions 354 and 391 of SEQ ID NO: 1, optionally wherein the amino acid substitution at position 354 of SEQ ID NO: 1 is L354I and the amino acid substitution at position 391 of SEQ ID NO: 1 is L391F;
v. the two amino acid substitutions are at positions 354 and 384 of SEQ ID NO: 1, optionally wherein the amino acid substitution at position 354 of SEQ ID NO: 1 is L354I and the amino acid substitution at position 384 of SEQ ID NO: 1 is L384M;
vi. the two amino acid substitutions are at positions 354 and 387 of SEQ ID NO: 1, optionally wherein the amino acid substitution at position 354 of SEQ ID NO: 1 is L354I and the amino acid substitution at position 387 of SEQ ID NO: 1 is L387M;
vii. the two amino acid substitutions are at positions 387 and 391, optionally wherein the amino acid substitution at position 387 of SEQ ID NO: 1 is L387M and the amino acid substitution at position 391 of SEQ ID NO: 1 is L391F;
viii. the two amino acid substitutions are at positions 384 and 387 of SEQ ID NO: 1, optionally wherein the amino acid substitution at position 384 of SEQ ID NO: 1 is L384M and the amino acid substitution at position 387 of SEQ ID NO: 1 is L387M; or
ix. the two amino acid substitutions are at positions 384 and 391 of SEQ ID NO: 1, optionally wherein the amino acid substitution at position 384 of SEQ ID NO: 1 is L384M and the amino acid substitution at position 391 of SEQ ID NO: 1 is L391F;
b. the one or more additional amino acid substitutions of the polypeptide, the first polypeptide monomer, and/or the second polypeptide monomer are three amino acid substitutions, optionally wherein each of the three amino acid substitutions are at a position of SEQ ID NO: 1 selected from: 343, 347, 351, 354, 388, 391, 404, 414, 418, 463, 521, 524, and 525, optionally wherein:
i. the three amino acid substitutions are at positions 354, 384, and 391 of SEQ ID NO: 1, optionally wherein the amino acid substitution at position 354 of SEQ ID NO: 1 is L354I, the amino acid substitution at position 384 of SEQ ID NO: 1 is L384M, and the amino acid substitution at position 391 of SEQ ID NO: 1 is L391F;
ii. the three amino acid substitutions are at positions 414, 463, and 524 of SEQ ID NO: 1, optionally wherein the amino acid substitution at position 414 of SEQ ID NO: 1 is Q414E, the amino acid substitution at position 463 of SEQ ID NO: 1 is S463P, and the amino acid substitution at position 524 of SEQ ID NO: 1 is H524L;
c. the one or more additional amino acid substitutions of the polypeptide, the first polypeptide monomer, and/or the second polypeptide monomer are four amino acid substitutions, optionally wherein each of the four amino acid substitutions are at a position of SEQ ID NO: 1 selected from: 343, 347, 351, 354, 384, 388, 391, 404, 413, 418, 463, 521, 524, and 525, optionally wherein:
i. the four amino acid substitutions are at positions 354, 384, 391, and 418 of SEQ ID NO: 1, optionally wherein the amino acid substitution at position 354 of SEQ ID NO: 1 is L354I, the amino acid substitution at position 384 of SEQ ID NO: 1 is L384M, the amino acid substitution at position 391 of SEQ ID NO: 1 is L391F, and the amino acid substitution at position 418 of SEQ ID NO: 1 is V418I;
ii. the four amino acid substitutions are at positions 343, 388, 521, and 404 of SEQ ID NO: 1, optionally wherein the amino acid substitution at position 343 of SEQ ID NO: 1 is M343I, the amino acid substitution at position 388 of SEQ ID NO: 1 is M388I, the amino acid substitution at position 521 of SEQ ID NO: 1 is G521I, and the amino acid substitution at position 404 of SEQ ID NO: 1 is F404L;
iii. the four amino acid substitutions are at positions 524, 347, 351, and 525 of SEQ ID NO: 1, optionally wherein the amino acid substitution at position 524 of SEQ ID NO: 1 is H524V, the amino acid substitution at position 347 of SEQ ID NO: 1 is T347R, the amino acid substitution at position 351 of SEQ ID NO: 1 is D351Q, and the amino acid substitution at position 525 of SEQ ID NO: 1 is L525N;
iv. the four amino acid substitutions are at positions 354, 384, 391, and 463 of SEQ ID NO: 1, optionally wherein the amino acid substitution at position 354 of SEQ ID NO: 1 is L354I, the amino acid substitution at position 384 of SEQ ID NO: 1 is L384M, the amino acid substitution at position 391 of SEQ ID NO: 1 is L391V, and the amino acid substitution at position 463 of SEQ ID NO: 1 is S463P;
v. the four amino acid substitutions are at positions 384, 391, 413, and 524 of SEQ ID NO: 1, optionally wherein the amino acid substitution at position 384 of SEQ ID NO: 1 is L384M, the amino acid substitution at position 391 of SEQ ID NO: 1 is L391V, the amino acid substitution at position 413 of SEQ ID NO: 1 is N413D, and the amino acid substitution at position 524 of SEQ ID NO: 1 is H524F;
d. the one or more additional amino acid substitutions of the polypeptide, the first polypeptide monomer, and/or the second polypeptide monomer are five amino acid substitutions, optionally wherein each of the five amino acid substitutions are at a position of SEQ ID NO: 1 selected from: 354, 384, 391, 409, 413, 414, 421, 463, and 524, optionally wherein
i. the five amino acid substitutions are at positions 384, 409, 413, 463, and 524 of SEQ ID NO: 1, optionally wherein the amino acid substitution at position 384 of SEQ ID NO: 1 is L384M, the amino acid substitution at position 409 of SEQ ID NO: 1 is L409V, the amino acid substitution at position 413 of SEQ ID NO: 1 is N413D, the amino acid substitution at position 463 of SEQ ID NO: 1 is S463P, and the amino acid substitution at position 524 of SEQ ID NO: 1 is H524L;
ii. the five amino acid substitutions are at positions 391, 413, 414, 463, and 524 of SEQ ID NO: 1, optionally wherein the amino acid substitution at position 391 of SEQ ID NO: 1 is L391V, the amino acid substitution at position 413 of SEQ ID NO: 1 is N413D, the amino acid substitution at position 414 of SEQ ID NO: 1 is Q414E, the amino acid substitution at position 463 of SEQ ID NO: 1 is S463P, and the amino acid substitution at position 524 of SEQ ID NO: 1 is H524F;
iii. the five amino acid substitutions are at positions 391, 414, 421, 463, and 524 of SEQ ID NO: 1, optionally wherein the amino acid substitution at position 391 of SEQ ID NO: 1 is L391V, the amino acid substitution at position 414 of SEQ ID NO: 1 is Q414E, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524F;
iv. the five amino acid substitutions are at positions 354, 409, 413, 421, and 524 of SEQ ID NO: 1, optionally wherein the amino acid substitution at position 354 of SEQ ID NO: 1 is L354I, the amino acid substitution at position 409 of SEQ ID NO: 1 is L409V, the amino acid substitution at position 413 of SEQ ID NO: 1 is N413D, the amino acid substitution at position 421 of SEQ ID NO: 1 is M421L, and the amino acid substitution at position 524 of SEQ ID NO: 1 is H524L; or
v. the five amino acid substitutions are at positions 354, 409, 421, 463, and 524 of SEQ ID NO: 1, optionally wherein the amino acid substitution at position 354 of SEQ ID NO: 1 is L354I, the amino acid substitution at position 409 of SEQ ID NO: 1 is L409V, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524L;
e. the one or more additional amino acid substitutions of the polypeptide, the first polypeptide monomer, and/or the second polypeptide monomer are six amino acid substitutions, optionally wherein each of the six amino acid substitutions are at a position of SEQ ID NO: 1 selected from: 354, 384, 391, 409, 413, 414, 421, 463, and 524, optionally wherein:
i. the six amino acid substitutions are at positions 384, 391, 413, 421, 463, and 524 of SEQ ID NO: 1, optionally wherein the amino acid substitution at position 384 of SEQ ID NO: 1 is L384M, the amino acid substitution at position 391 of SEQ ID NO: 1 is L391V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524L;
ii. the six amino acid substitutions are at positions 409, 413, 414, 421, 463, and 524 of SEQ ID NO: 1; optionally wherein the amino acid substitution at position 409 of SEQ ID NO: 1 is L409V, the amino acid substitution at position 413 of SEQ ID NO: 1 is N413D, the amino acid substitution at position 414 of SEQ ID NO: 1 is Q414E, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524L;
iii. the six amino acid substitutions are at positions 354, 391, 409, 413, 414, and 524 of SEQ ID NO: 1, optionally wherein the amino acid substitution at position 354 of SEQ ID NO: 1 is L354I, the amino acid substitution at position 391 of SEQ ID NO: 1 is L391V, the amino acid substitution at position 409 of SEQ ID NO: 1 is L409V, the amino acid substitution at position 413 of SEQ ID NO: 1 is N413D, the amino acid substitution at position 414 of SEQ ID NO: 1 is Q414E, and the amino acid substitution at position 524 of SEQ ID NO: 1 is H524L;
f. the one or more additional amino acid substitutions of the polypeptide, the first polypeptide monomer, and/or the second polypeptide monomer are seven amino acid substitutions, optionally wherein each of the seven amino acid substitutions are at a position of SEQ ID NO: 1 selected from: 354, 384, 391, 409, 413, 414, 421, 463, 517, and 524, optionally wherein:
i. the seven amino acid substitutions are at positions 354, 384, 409, 413, 421, 463, and 524 of SEQ ID NO: 1, optionally wherein the amino acid substitution at position 354 of SEQ ID NO: 1 is L354I, the amino acid substitution at position 384 of SEQ ID NO: 1 is L384M, the amino acid substitution at position 409 of SEQ ID NO: 1 is L409V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, and the amino acid substitution at position 524 of SEQ ID NO: 1 is H524F;
ii. the seven amino acid substitutions are at positions 354, 391, 413, 421, 463, 517, and 524 of SEQ ID NO: 1, optionally wherein the amino acid substitution at position 354 of SEQ ID NO: 1 is L354I, the amino acid substitution at position 391 of SEQ ID NO: 1 is L391V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, the amino acid substitution at position 517 of SEQ ID NO: 1 is M517A, and the amino acid substitution at position 524 of SEQ ID NO: 1 is H524L;
iii. the seven amino acid substitutions are at positions 354, 391, 413, 414, 421, 517, and 524 of SEQ ID NO: 1; optionally wherein the amino acid substitution at position 354 of SEQ ID NO: 1 is L354I, the amino acid substitution at position 391 of SEQ ID NO: 1 is L391V, the amino acid substitution at position 413 of SEQ ID NO: 1 is N413D, the amino acid substitution at position 414 of SEQ ID NO: 1 is Q414E, the amino acid substitution at position 421 of SEQ ID NO: 1 is M421L, the amino acid substitution at position 517 of SEQ ID NO: 1 is M517A, and the amino acid substitution at position 524 of SEQ ID NO: 1 is H524F; or
g. the one or more additional amino acid substitutions of the polypeptide, the first polypeptide monomer, and/or the second polypeptide monomer are eight amino acid substitutions, optionally wherein the eight amino acid substitutions are at positions 384, 391, 409, 413, 421, 463, 517, and 524 of SEQ ID NO: 1, optionally wherein
i. the amino acid substitution at position 384 of SEQ ID NO: 1 is L384M, the amino acid substitution at position 391 of SEQ ID NO: 1 is L391V, the amino acid substitution at position 409 of SEQ ID NO: 1 is L409V, the amino acid substitution at position 413 of SEQ ID NO: 1 is N413D, the amino acid substitution at position 421 of SEQ ID NO: 1 is M421L, the amino acid substitution at position 463 of SEQ ID NO: 1 is S463P, the amino acid substitution at position 517 of SEQ ID NO: 1 is M517A, and the amino acid substitution at position 524 of SEQ ID NO: 1 is H524F.

5. An inducible cell death system comprising a first polypeptide and a second polypeptide monomer, wherein the first and the second polypeptide monomers each comprise a ligand binding domain and a cell death inducing domain, wherein the first and the second polypeptide monomers are configured to oligomerize upon contact with a ligand of the ligand binding domain, thereby generating a cell-death inducing signal in a cell in which the first and the second polypeptide monomers are expressed, and wherein
the ligand binding domain comprises a modified estrogen receptor ligand binding domain (ER-LBD) comprising an amino acid sequence corresponding to a hormone binding domain of a reference human estrogen receptor sequence (SEQ ID NO: 1), wherein the modified ER-LBD comprises:
(a) a G400V amino acid substitution, an M543A amino acid substitution, an L544A amino acid substitution, and optionally a V595A amino acid substitution, with reference to SEQ ID NO: 1; and
(b) additional amino acid substitutions, wherein the additional amino acid substitutions comprise, with reference to SEQ ID NO: 1:
(i) an L384M substitution, an L391V substitution, a N413D substitution, an M421L substitution, a S463P substitution, and a H524L substitution,
(ii) an L391V substitution, a N413D substitution, a Q414E substitution, a S463P substitution, and a H524F substitution,
(iii) an L354I substitution, a L391V substitution, a N413D substitution, a Q414E substitution, a M421L substitution, a M517A substitution, and a H524F substitution, or
(iv) an L354I substitution, a L391V substitution, a L409V substitution, a N413D substitution, a Q414E substitution, and a H524L substitution.

6. The inducible cell death system of claim 5, wherein the one or more additional amino acid substitutions of the polypeptide, the first polypeptide monomer, and/or the second polypeptide monomer comprises an N413D mutation, an H524 substitution, and an S463P substitution, optionally wherein:
a. the one or more additional amino acid substitutions of the polypeptide, the first polypeptide monomer, and/or the second polypeptide monomer comprise an L391V substitution, an L409V substitution, an Q414E substitution, an N413D substitution, an S463P substitution, an M517A substitution, and an H524L substitution, optionally wherein: the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 90 or 103;
b. the one or more additional amino acid substitutions of the polypeptide, the first polypeptide monomer, and/or the second polypeptide monomer comprise an L409V substitution, an N413D substitution, an S463P substitution, an M421L substitution, an L384M substitution, and an H524L substitution, optionally wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 91 or 104;
c. the one or more additional amino acid substitutions of the polypeptide, the first polypeptide monomer, and/or the second polypeptide monomer comprise an L391V substitution, an L409V substitution, an N413D substitution, an S463P substitution, an M517A substitution, an M421L substitution, an L354I substitution, and an H524L substitution, optionally wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 92 or 105;
d. the one or more additional amino acid substitutions of the polypeptide, the first polypeptide monomer, and/or the second polypeptide monomer comprise an L391V substitution, an Q414E substitution, an N413D substitution, an S463P substitution, an M421L substitution, an L354I substitution, an L384M substitution, and an H524L substitution, optionally wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 93 or 106;
e. the one or more additional amino acid substitutions of the polypeptide, the first polypeptide monomer, and/or the second polypeptide monomer comprise an L391V substitution, an L409V substitution, an N413D substitution, an S463P substitution, an M517A substitution, an M421L substitution, and an H524L substitution, optionally wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 94 or 107;
f. the one or more additional amino acid substitutions of the polypeptide, the first polypeptide monomer, and/or the second polypeptide monomer comprise an L391V substitution, an L409V substitution, an Q414E substitution, an N413D substitution, an S463P substitution, an L354I substitution, and an H524L substitution, optionally wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 95 or 108;
g. the one or more additional amino acid substitutions of the polypeptide, the first polypeptide monomer, and/or the second polypeptide monomer comprise an L391V substitution, an L409V substitution, an N413D substitution, an S463P substitution, an M421L substitution, an L354I substitution, an L384M substitution, and an H524L substitution, optionally wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 96 or 109;
h. the one or more additional amino acid substitutions of the polypeptide, the first polypeptide monomer, and/or the second polypeptide monomer comprise an L391V substitution, an Q414E substitution, an N413D substitution, an S463P substitution, an M517A substitution, an M421L substitution, an L354I substitution, and an H524L substitution, optionally wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 97 or 110;
i. the one or more additional amino acid substitutions of the polypeptide, the first polypeptide monomer, and/or the second polypeptide monomer comprise an L391V substitution, an N413D substitution, an S463P substitution, an M517A substitution, an L384M substitution, and an H524L substitution, optionally wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 98 or 111;
j. the one or more additional amino acid substitutions of the polypeptide, the first polypeptide monomer, and/or the second polypeptide monomer comprise an L391V substitution, an L409V substitution, an N413D substitution, an S463P substitution, an M517A substitution, and an H524L substitution, optionally wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 99 or 112;
k. the one or more additional amino acid substitutions of the polypeptide, the first polypeptide monomer, and/or the second polypeptide monomer comprise an N413D substitution, an S463P substitution, an L354I substitution, an L384M substitution, and an H524L substitution, optionally wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 100 or 113; or
l. the one or more additional amino acid substitutions of the polypeptide, the first polypeptide monomer, and/or the second polypeptide monomer comprise an N413D substitution, an S463P substitution, an M421L substitution, an L354I substitution, and an H524L substitution, optionally wherein the polypeptide, the first polypeptide monomer, and/or the second polypeptide monomer comprises an amino acid sequence at least 80%, 85%, 90%, 95%, 97.5%, 98%, 99%, or 100% identical to SEQ ID NO: 101 or 114, optionally wherein the ligand is a non-endogenous ligand, optionally wherein the non-endogenous ligand is selected from: 4-hydroxytamoxifen, N-desmethyltamoxifen, tamoxifen-N-oxide, and endoxifen, optionally wherein the non-endogenous ligand comprises a tamoxifen metabolite, optionally wherein the non-endogenous ligand is endoxifen.

7. The inducible cell death system of claim 5, wherein the first polypeptide monomer and the second polypeptide monomer are capable of oligomerization and/or generating the cell-death inducing signal at a concentration of:
a. 0.25 nM Endoxifen or less and/or at a concentration of 0.04 nM 4-OHT or less;
b. 2.5 nM Endoxifen or less and/or at a concentration of 0.4 nM 4-OHT or less;
c. at least 0.001 pM of 4-OHT; or
d. at least 0.01 pM of 4-OHT.

8. The inducible cell death system of claim 1, wherein the cell death-inducing domain is:
a. derived from a protein selected from: caspase 3, caspase 6, caspase 7, caspase 8, caspase 9, Diphtheria toxin fragment A (DTA), Bax, Bak, Bok, Bad, Bcl-Xs, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B1, and Purine nucleoside phosphorylase, optionally wherein the cell death-inducing domain comprises the Caspase 9 derived amino acid sequence of SEQ ID NO:48 or 125, optionally wherein the caspase domain or functional fragment thereof does not comprise a Caspase Activation and Recruitment Domain (CARD) domain sequence; or
b. a transcription factor comprising a nucleic acid-binding domain and a transcriptional effector domain, wherein the transcription factor is configured to generate a cell-death inducing signal by inducing expression of: a caspase domain or functional fragment thereof, optionally wherein the caspase is selected from caspase 3, caspase 6, caspase 7, caspase 8, caspase 9, or functional fragments thereof, respectively, Diphtheria toxin fragment A (DTA), Bax, Bak, Bok, Bad, Bcl-Xs, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B1, or Purine nucleoside phosphorylase.

9. An isolated polynucleotide comprising a nucleotide sequence encoding the polypeptide of claim 1.

10. A heterologous construct comprising a promoter operatively linked to the polynucleotide of claim 9.

11. A plasmid or a vector comprising the heterologous construct of claim 10.

12. A cell comprising the heterologous construct of claim 10.

13. A molecular switch for generating a cell-death inducing signal in a cell, comprising:
(a) the inducible cell death system of claim 1, wherein the inducible cell death system is capable of generating a cell-death inducing signal in the cell; and
(b) a non-endogenous ligand, wherein binding of the non-endogenous ligand to the modified ER-LBD generates the cell-death inducing signal in the cell, optionally wherein the non-endogenous ligand is selected from: 4-hydroxytamoxifen, N-desmethyltamoxifen, tamoxifen-N-oxide, and endoxifen, optionally wherein the non-endogenous ligand comprises a tamoxifen metabolite, optionally wherein the non-endogenous ligand is endoxifen, optionally wherein:
a. the first polypeptide monomer and the second polypeptide monomer are capable of oligomerization and/or generating the cell-death inducing signal at a concentration of 0.25 nM Endoxifen or less and/or at a concentration of 0.04 nM 4-OHT or less;
b. the first polypeptide monomer and the second polypeptide monomer are capable of oligomerization and/or generating the cell-death inducing signal at a concentration of 2.5 nM Endoxifen or less and/or at a concentration of 0.4 nM 4-OHT or less;
c. the first polypeptide monomer and the second polypeptide monomer are capable of oligomerization and/or generating the cell-death inducing signal at a concentration of at least 0.001 pM of 4-OHT; or
d. the first polypeptide monomer and the second polypeptide monomer are capable of oligomerization and/or generating the cell-death inducing signal at a concentration of at least 0.01 pM of 4-OHT.

14. A method of inducing oligomerization of a chimeric protein comprising: transforming a cell with (i) a heterologous construct encoding any one of the inducible cell death systems of claim 1, and (ii) contacting the transformed cell with a non-endogenous ligand of the modified estrogen receptor ligand binding domain (ER-LBD), optionally wherein
a. the method further comprising culturing the transformed cell under conditions suitable for expression of the of the inducible cell death system prior to inducing oligomerization and/or inducing cell death;
b. the transformed cell is in a human or animal, and wherein contacting the transformed cell with the non-endogenous ligand comprises administering a pharmacological dose of the ligand to the human or animal; and/or
c. the non-endogenous ligand is selected from: 4-hydroxytamoxifen, N-desmethyltamoxifen, tamoxifen-N-oxide, and endoxifen, optionally wherein the non-endogenous ligand comprises a tamoxifen metabolite, optionally wherein the non-endogenous ligand is endoxifen, optionally wherein the non-endogenous ligand is administered at a concentration at which the non-endogenous ligand is substantially inactive on a wild-type estrogen receptor alpha of SEQ ID NO: 1.

15. A modified estrogen receptor ligand binding domain (ER-LBD) corresponding to a hormone binding domain of a reference human estrogen receptor sequence (SEQ ID NO: 1), wherein:
a. the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an N413D substitution, an S463P substitution, an L354I substitution, an L384M substitution, and an H524L substitution, with reference to SEQ ID NO: 1;
b. the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an N413D substitution, an S463P substitution, an M421L substitution, an L354I substitution, and an H524L substitution, with reference to SEQ ID NO: 1;
c. the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L409V substitution, an N413D substitution, an S463P substitution, an M421L substitution, an L384M substitution, and an H524L substitution, with reference to SEQ ID NO: 1;
d. the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an L409V substitution, an Q414E substitution, an N413D substitution, an S463P substitution, an M517A substitution, and an H524L substitution, with reference to SEQ ID NO: 1;
e. the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an L409V substitution, an N413D substitution, an S463P substitution, an M517A substitution, an M421L substitution, an L354I substitution, and an H524L substitution, with reference to SEQ ID NO: 1;
f. the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an Q414E substitution, an N413D substitution, an S463P substitution, an M421L substitution, an L354I substitution, an L384M substitution, and an H524L substitution, with reference to SEQ ID NO: 1;
g. the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an L409V substitution, an N413D substitution, an S463P substitution, an M517A substitution, an M421L substitution, and an H524L substitution, with reference to SEQ ID NO: 1;
h. the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an L409V substitution, an Q414E substitution, an N413D substitution, an S463P substitution, an L354I substitution, and an H524L substitution, with reference to SEQ ID NO: 1;
i. the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an L409V substitution, an N413D substitution, an S463P substitution, an M421L substitution, an L354I substitution, an L384M substitution, and an H524L substitution, with reference to SEQ ID NO: 1;
j. the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an Q414E substitution, an N413D substitution, an S463P substitution, an M517A substitution, an M421L substitution, an L354I substitution, and an H524L substitution, with reference to SEQ ID NO: 1;
k. the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an N413D substitution, an S463P substitution, an M517A substitution, an L384M substitution, and an H524L substitution, with reference to SEQ ID NO: 1;
l. the modified ER-LBD comprises (a) a G400V amino acid substitution, an M543A amino acid substitution, an L544A amino acid substitution, and optionally a V595A amino acid substitution, with reference to SEQ ID NO: 1; and (b) one or more additional amino acid substitutions, wherein the one or more additional amino acid substitutions comprise: an L391V substitution, an L409V substitution, an N413D substitution, an S463P substitution, an M517A substitution, and an H524L substitution, with reference to SEQ ID NO: 1; or
m. the modified ER-LBD comprises an amino acid sequence having at least 80%, 85%, 90%, 95%, 97.5%, 98%, 99%, or 100% identical to any one of SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, and SEQ ID NO: 114,
optionally wherein the modified ER-LBD has greater sensitivity and/or selectivity to a non-endogenous ligand as compared to an ER-LBD comprising the amino acid sequence of SEQ ID NO: 2, or as compared to an endogenous ligand as a result of the one or more additional amino acid substitutions, optionally wherein the non-endogenous ligand is selected from the group consisting of: 4-hydroxytamoxifen, N-desmethyltamoxifen, tamoxifen-N-oxide, tamoxifen, and endoxifen, optionally wherein the endogenous ligand is estradiol, optionally wherein the modified ER-LBD further comprises a V595A amino acid substitution.

16. A chimeric protein comprising a polypeptide of interest fused to the modified ER-LBD of claim 15, optionally wherein the polypeptide of interest comprises a nucleic acid binding domain, optionally wherein the nucleic acid binding domain comprises a zinc finger domain, optionally wherein the zinc finger domain comprises the sequence as set forth in SEQ ID NO: 57 or SEQ ID NO: 84, optionally wherein the chimeric protein comprises a chimeric transcription factor, and wherein the polypeptide of interest comprises a nucleic acid binding domain and a transcriptional modulator domain, optionally wherein the transcriptional modular domain is a transcriptional activator, optionally wherein the transcriptional activator is selected from the group consisting of: a Herpes Simplex Virus Protein 16 (VP16) activation domain; an activation domain comprising four tandem copies of VP16; a VP64 activation domain; a p65 activation domain of NFκB (p65); an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase core domain of the human E1A-associated protein p300 (p300 HAT core activation domain). the transcriptional activator is selected from the group consisting of: a Herpes Simplex Virus Protein 16 (VP16) activation domain; an activation domain comprising four tandem copies of VP16; a VP64 activation domain; a p65 activation domain of NΓKB (p65); an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase core domain of the human E1A-associated protein p300 (p300 HAT core activation domain), optionally wherein the transcriptional activator is a p65 transcriptional activator comprising the amino acid sequence of
(SEQ ID NO: 64)
DEFPTMVFPSGQISQASALAPAPPQVLPQAPAPAPAPAMVSALAQAPAP
VPVLAPGPPQAVAPPAPKPTQAGEGTLSEALLQLQFDDEDLGALLGNST
DPAVFTDLASVDNSEFQQLLNQGIPVAPHTTEPMLMEYPEAITRLVTGA
QRPPDPAPAPLGAPGLPNGLLSGDEDFSSIADMDFSALLSQISS.

17. An isolated polynucleotide molecule comprising a nucleotide sequence encoding the modified ER-LBD of claim 15 or a chimeric protein thereof.

18. A heterologous construct comprising a promoter operatively linked to the polynucleotide molecule of claim 17.

19. A cell comprising the heterologous construct of claim 18.

20. A molecular switch for modulating transcription of a gene of interest, comprising:
a) the chimeric protein or a heterologous construct encoding the chimeric protein of claim 16, wherein the chimeric protein binds to a chimeric transcription factor-responsive (CTF-responsive) promoter operably linked to the gene of interest; and
b) a non-endogenous ligand, wherein binding of the non-endogenous ligand to the modified ER-LBD induces the chimeric protein to modulate transcription of the gene of interest,
optionally wherein:
a. the non-endogenous ligand is selected from: 4-hydroxytamoxifen, N-desmethyltamoxifen, tamoxifen-N-oxide, tamoxifen, and endoxifen;
b. the gene of interest encodes a polypeptide selected from the group consisting of: a cytokine, a chemokine, a homing molecule, a growth factor, a cell death regulator, a co-activation molecule, a tumor microenvironment modifier a, a receptor, a ligand, an antibody, a polynucleotide, a peptide, and an enzyme;
c. the molecular switch of further comprises an additional construct comprising the CTF-responsive promoter operably linked to the gene of interest;
d. the heterologous construct and the additional construct are comprised in a single vector; and/or
e. the heterologous construct is comprised in a first vector and the additional construct is comprised in a second vector.

21. A method of modulating localization of a chimeric protein, comprising:
a) transforming a cell with a heterologous construct encoding the chimeric protein of claim 16; and
b) inducing nuclear localization of the chimeric protein by contacting the transformed cell with a non-endogenous ligand,
optionally wherein the method further comprises culturing the transformed cell under conditions suitable for expression of the chimeric protein prior to contacting the transformed cell with the non-endogenous ligand, and/or optionally wherein the heterologous construct and the additional construct are comprised in a single vector or the heterologous construct is comprised in a first vector and the additional construct is comprised in a second vector, and/or optionally wherein the non-endogenous ligand is selected from the group consisting of: 4-hydroxytamoxifen, N-desmethyltamoxifen, tamoxifen-N-oxide, tamoxifen, and endoxifen, and/or optionally wherein the non-endogenous ligand is administered at a concentration at which the non-endogenous ligand is substantially inactive on a wild-type estrogen receptor alpha of SEQ ID NO: 1.
